(19) United States
(12) Patent Application Publication
(10) Pub. No.: US 2025/0161434 A1
(43) Pub. Date: May 22, 2025

(54) CORONAVIRUS VACCINE COMPOSITIONS AND METHODS

(71) Applicant: Arcturus Therapeutics, Inc., San Diego, CA (US)

(72) Inventors: Sean Michael SULLIVAN, Escondido, CA (US); Daiki MATSUDA, San Diego, CA (US); Kiyoshi TACHIKAWA, San Diego, CA (US); Padmanabh CHIVUKULA, San Diego, CA (US); Priya Prakash KARMALI, San Diego, CA (US); Jared Henry DAVIS, Poway, CA (US); Yanjie BAO, San Diego, CA (US); Amit SAGI, San Diego, CA (US)

(21) Appl. No.: 18/969,110

(22) Filed: Dec. 4, 2024

Related U.S. Application Data
(63) Continuation of application No. 18/345,893, filed on Jun. 30, 2023, now Pat. No. 12,220,455.
Application No. 18/345,893 is a continuation of application No. 17/196,889, filed on Mar. 9, 2021, now Pat. No. 11,744,887.
(60) Provisional application No. 63/073,900, filed on Sep. 2, 2020.
(60) Provisional application No. 62/987,191, filed on Mar. 9, 2020.

(51) Int. Cl. A61K 39/215; A61K 9/51; A61K 38/00; A61K 39/00; A61K 39/12; A61K 47/10; A61K 47/20; A61K 47/26; C07K 14/005; C07K 14/18; C12N 7/00; C12N 15/86

(52) U.S. Cl. CPC A61K 39/215; A61K 9/5123; A61K 39/12; A61K 47/10; A61K 47/20; A61K 47/26; C07K 14/005; C07K 14/1808; C12N 7/00; C12N 15/86; A61K 38/00; A61K 2039/53; C12N 2770/20022; C12N 2770/20034; C12N 2770/36122; C12N 2770/36134; C12N 2830/42; C12N 2830/50

(57) ABSTRACT

Provided herein are nucleic acid molecules encoding viral replication proteins and antigenic coronavirus proteins or fragments thereof. Also provided herein are compositions that include nucleic acid molecules encoding viral replication and antigenic proteins, and lipids. Nucleic acid molecules provided herein are useful for inducing immune responses.

### CROSS-REFERENCES TO RELATED APPLICATIONS

This application claims the benefit of U.S. Provisional Application No. 62/987,191, filed Mar. 9, 2020 and U.S. Provisional Application No. 63/073,900, filed Sep. 2, 2020.

### REFERENCE TO A SEQUENCE LISTING

The instant application contains a Sequence Listing which has been submitted electronically in ASCII format and is hereby incorporated by reference in its entirety. Said ASCII copy, created on Mar. 8, 2021 is named 049386-538001US_SequenceListing_ST25.txt and is 481,150 bytes in size.

### TECHNICAL FIELD

The present disclosure relates generally to inducing immune responses against infectious agents and tumor antigens and more specifically to self-transcribing and replicating RNA for antigen expression.

### BACKGROUND

Infectious diseases and cancer represent significant burdens on health worldwide. According to the World Health Organization (WHO), lower respiratory tract infection was the deadliest infectious disease worldwide in 2016, causing approximately 3 million deaths. The impact of infectious diseases is illustrated by the coronavirus disease 2019 (COVID-19) pandemic caused by severe acute respiratory syndrome-coronavirus-2 (SARS-CoV-2). SARS-CoV-2 is a novel coronavirus that was first identified in December 2019 in Wuhan, China and that has caused more than 20 million confirmed infections with more than 700,000 deaths worldwide as of August 2020. Current control measures to curb the rapid worldwide spread of SARS-CoV-2, such as national lockdowns, closure of work places and schools, and reduction of international travel are threatening to result in a global economic recession to an extent not seen since the Great Depression.

Cancer is the second leading cause of death globally, accounting for approximately 9.6 million deaths worldwide in 2018. Cancer is a large group of diseases that can affect almost any organ or tissue in the body. Cancer burden continues to grow globally, exerting physical, emotional, and financial strains on patients and health care providers.

Self-replicating ribonucleic acids (RNAs), e.g., derived from viral replicons, are useful for expression of proteins, such as heterologous proteins, for a variety of purposes, such as expression of therapeutic proteins and expression of antigens for vaccines. A desirable property of such replicons is the ability for sustained expression of the protein.

Few treatments for infections caused by viruses and eukaryotic organisms are available, and resistance to antibiotics for the treatment of bacterial infections is increasing. In addition, rapid responses, including rapid vaccine development, are required to effectively control emerging infectious diseases and pandemics. Moreover, many cancer treatments include costly and painful surgeries and chemotherapies that are often unsuccessful or only modestly prolong life despite serious side effects. Thus, there exists a need for the prevention and/or treatment of infectious diseases and cancer.

### SUMMARY

In one aspect, the present disclosure provides a nucleic acid molecule comprising (i) a first polynucleotide encoding one or more viral replication proteins, wherein the first polynucleotide is codon-optimized as compared to a wild-type polynucleotide encoding the one or more viral replication proteins; and (ii) a second polynucleotide comprising a first transgene encoding a first antigenic protein or a fragment thereof, wherein the first antigenic protein is a coronavirus protein.

In some embodiments, the one or more viral replication proteins are alphavirus proteins or rubivirus proteins.

In some embodiments, the alphavirus proteins are from Venezuelan Equine Encephalitis Virus (VEEV), Eastern Equine Encephalitis Virus (EEEV), Everglades Virus (EVEV), Mucambo Virus (MUCV), Semliki Forest Virus (SFV), Pixuna Virus (PIXV), Middleburg Virus (MIDV), Chikungunya Virus (CHIKV), O'Nyong-Nyong Virus (ONNV), Ross River Virus (RRV), Barmah Forest Virus (BFV), Getah Virus (GETV), Sagiyama Virus (SAGV), Bebaru Virus (BEBV), Mayaro Virus (MAYV), Una Virus (UNAV), Sindbis Virus (SINV), Aura Virus (AURAV), Whataroa Virus (WHAV), Babanki Virus (BABV), Kyzylagach Virus (KYZV), Western Equine Encephalitis Virus (WEEV), Highland J Virus (HJV), Fort Morgan Virus (FMV), Ndumu Virus (NDUV), Salmonid Alphavirus (SAV), Buggy Creek Virus (BCRV), or any combination thereof.

In some embodiments, the first polynucleotide encodes a polyprotein comprising an alphavirus nsP1 protein, an alphavirus nsP2 protein, an alphavirus nsP3 protein, an alphavirus nsP4 protein, or any combination thereof.

In some embodiments, the first polynucleotide encodes a polyprotein comprising an alphavirus nsP1 protein, an alphavirus nsP2 protein, an alphavirus nsP3 protein, or any combination thereof, and an alphavirus nsP4 protein.

In some embodiments, the nucleic acid molecule further comprises a first intergenic region between a sequence encoding the polyprotein comprising an alphavirus nsP1 protein, an alphavirus nsP2 protein, an alphavirus nsP3 protein, or any combination thereof, and a sequence encoding an alphavirus nsP4 protein.

In some embodiments, the first intergenic region comprises an alphavirus sequence.

In some embodiments, the first polynucleotide comprises a sequence having at least 80% identity to a sequence of SEQ ID NO:72.

In some embodiments, the nucleic acid molecule further comprises a 5′ untranslated region (UTR).

In some embodiments, the 5′ UTR comprises a viral 5′ UTR, a non-viral 5′ UTR, or a combination of viral and non-viral 5′ UTR sequences.

In some embodiments, the 5′ UTR comprises an alphavirus 5′ UTR.

In some embodiments, the alphavirus 5′ UTR comprises a Venezuelan Equine Encephalitis Virus (VEEV), Eastern Equine Encephalitis Virus (EEEV), Everglades Virus (EVEV), Mucambo Virus (MUCV), Semliki Forest Virus (SFV), Pixuna Virus (PIXV), Middleburg Virus (MIDV), Chikungunya Virus (CHIKV), O'Nyong-Nyong Virus (ONNV), Ross River Virus (RRV), Barmah Forest Virus (BFV), Getah Virus (GETV), Sagiyama Virus (SAGV), Bebaru Virus (BEBV), Mayaro Virus (MAYV), Una Virus (UNAV), Sindbis Virus (SINV), Aura Virus (AURAV), Whataroa Virus (WHAV), Babanki Virus (BABV), Kyzylagach Virus (KYZV), Western Equine Encephalitis Virus (WEEV), Highland J Virus (HJV), Fort Morgan Virus (FMV), Ndumu Virus (NDUV), Salmonid Alphavirus (SAV), or Buggy Creek Virus (BCRV) 5′ UTR sequence.

In some embodiments, the 5′ UTR comprises a sequence of SEQ ID NO:73, SEQ ID NO:74, or SEQ ID NO:75.

In some embodiments, the nucleic acid molecule further comprises a 3′ untranslated region (UTR).

In some embodiments, the 3′ UTR comprises a viral 3′ UTR, a non-viral 3′ UTR, or a combination of viral and non-viral 3′ UTR sequences. In some embodiments, the 3′ UTR comprises an alphavirus 3′ UTR.

In some embodiments, the alphavirus 3′ UTR comprises a Venezuelan Equine Encephalitis Virus (VEEV), Eastern Equine Encephalitis Virus (EEEV), Everglades Virus (EVEV), Mucambo Virus (MUCV), Semliki Forest Virus (SFV), Pixuna Virus (PIXV), Middleburg Virus (MIDV), Chikungunya Virus (CHIKV), O'Nyong-Nyong Virus (ONNV), Ross River Virus (RRV), Barmah Forest Virus (BFV), Getah Virus (GETV), Sagiyama Virus (SAGV), Bebaru Virus (BEBV), Mayaro Virus (MAYV), Una Virus (UNAV), Sindbis Virus (SINV), Aura Virus (AURAV), Whataroa Virus (WHAV), Babanki Virus (BABV), Kyzylagach Virus (KYZV), Western Equine Encephalitis Virus (WEEV), Highland J Virus (HJV), Fort Morgan Virus (FMV), Ndumu Virus (NDUV), Salmonid Alphavirus (SAV), or Buggy Creek Virus (BCRV) 3′ UTR sequence.

In some embodiments, the 3′ UTR comprises a poly-A sequence.

In some embodiments, the 3′ UTR comprises a sequence of SEQ ID NO:76.

In some embodiments, the antigenic protein is a SARS-CoV-2 protein.

In some embodiments, the antigenic protein is a SARS-CoV-2 spike glycoprotein.

In some embodiments, the SARS-CoV-2 spike glycoprotein is a wild-type SARS-CoV-2 spike glycoprotein having an amino acid sequence of SEQ ID NO:123.

In some embodiments, the second polynucleotide comprises a sequence having at least 85% identity to a sequence of SEQ ID NO:121 or SEQ ID NO:122.

In some embodiments, the second polynucleotide comprises at least two transgenes.

In some embodiments, a second transgene encodes a second antigenic protein or a fragment thereof or an immunomodulatory protein.

In some embodiments, the second polynucleotide further comprises a sequence encoding a 2A peptide, an internal ribosomal entry site (IRES), or a combination thereof, located between transgenes.

In some embodiments, the immunomodulatory protein is a cytokine, a chemokine, or an interleukin.

In some embodiments, the second transgene encodes a second coronavirus protein.

In some embodiments, the first polynucleotide is located 5′ of the second polynucleotide.

In some embodiments, the nucleic acid molecule further comprises a second intergenic region located between the first polynucleotide and the second polynucleotide.

In some embodiments, the second intergenic region comprises a sequence having at least 85% identity to a sequence of SEQ ID NO:77.

In some embodiments, the nucleic acid molecule is
(a) a DNA molecule; or
(b) an RNA molecule, wherein T is substituted with U.

In some embodiments, the DNA molecule further comprises a promoter.

In some embodiments, the promoter is located 5′ of the 5′UTR.

In some embodiments, the promoter is a T7 promoter, a T3 promoter, or an SP6 promoter.

In some embodiments, the RNA molecule is a self-replicating RNA molecule.

In some embodiments, the RNA molecule further comprises a 5′ cap.

In some embodiments, the 5′ cap has a Cap 1 structure, a Cap 1 (^{m6}A) structure, a Cap 2 structure, a Cap 0 structure, or any combination thereof.

In another aspect, the disclosure provides a nucleic acid molecule comprising
(a) a sequence of SEQ ID NO:124;
(b) a sequence of SEQ ID NO:124, wherein T is substituted with U;
(c) a sequence of SEQ ID NO:125; or
(d) a sequence of SEQ ID NO:125, wherein T is substituted with U.

In some embodiments, the nucleic acid molecule is an RNA molecule.

In some embodiments, the nucleic acid molecule further comprises a 5′ cap having a Cap 1 structure.

In yet another aspect the disclosure provides a nucleic acid molecule comprising:
(i) a first polynucleotide comprising a sequence having at least 80% identity to a sequence of SEQ ID NO:72; and
(ii) a second polynucleotide comprising a first transgene encoding a first antigenic protein or a fragment thereof, wherein the first antigenic protein is a coronavirus protein.

In some embodiments, the nucleic acid molecule further comprises a 5′ untranslated region (UTR).

In some embodiments, the 5′ UTR comprises a viral 5′ UTR, a non-viral 5′ UTR, or a combination of viral and non-viral 5′ UTR sequences.

In some embodiments, the 5′ UTR comprises an alphavirus 5′ UTR.

In some embodiments, the alphavirus 5′ UTR comprises a Venezuelan Equine Encephalitis Virus (VEEV), Eastern Equine Encephalitis Virus (EEEV), Everglades Virus (EVEV), Mucambo Virus (MUCV), Semliki Forest Virus (SFV), Pixuna Virus (PIXV), Middleburg Virus (MIDV), Chikungunya Virus (CHIKV), O'Nyong-Nyong Virus (ONNV), Ross River Virus (RRV), Barmah Forest Virus (BFV), Getah Virus (GETV), Sagiyama Virus (SAGV), Bebaru Virus (BEBV), Mayaro Virus (MAYV), Una Virus (UNAV), Sindbis Virus (SINV), Aura Virus (AURAV), Whataroa Virus (WHAV), Babanki Virus (BABV), Kyzylagach Virus (KYZV), Western Equine Encephalitis Virus (WEEV), Highland J Virus (HJV), Fort Morgan Virus (FMV), Ndumu Virus (NDUV), Salmonid Alphavirus (SAV), or Buggy Creek Virus (BCRV) 5′ UTR sequence.

In some embodiments, the 5′ UTR comprises a sequence of SEQ ID NO:73, SEQ ID NO:74, or SEQ ID NO:75.

In some embodiments, the nucleic acid molecule further comprises a 3′ untranslated region (UTR).

In some embodiments, the 3′ UTR comprises a viral 3′ UTR, a non-viral 3′ UTR, or a combination of viral and non-viral 3′ UTR sequences.

In some embodiments, the 3′ UTR comprises an alphavirus 3′ UTR.

In some embodiments, the alphavirus 3′ UTR comprises a Venezuelan Equine Encephalitis Virus (VEEV), Eastern Equine Encephalitis Virus (EEEV), Everglades Virus (EVEV), Mucambo Virus (MUCV), Semliki Forest Virus (SFV), Pixuna Virus (PIXV), Middleburg Virus (MIDV), Chikungunya Virus (CHIKV), O'Nyong-Nyong Virus (ONNV), Ross River Virus (RRV), Barmah Forest Virus (BFV), Getah Virus (GETV), Sagiyama Virus (SAGV), Bebaru Virus (BEBV), Mayaro Virus (MAYV), Una Virus (UNAV), Sindbis Virus (SINV), Aura Virus (AURAV), Whataroa Virus (WHAV), Babanki Virus (BABV), Kyzylagach Virus (KYZV), Western Equine Encephalitis Virus (WEEV), Highland J Virus (HJV), Fort Morgan Virus (FMV), Ndumu Virus (NDUV), Salmonid Alphavirus (SAV), or Buggy Creek Virus (BCRV) 3′ UTR sequence.

In some embodiments, the 3′ UTR comprises a poly-A sequence.

In some embodiments, the 3′ UTR comprises a sequence of SEQ ID NO:76.

In some embodiments, the antigenic protein is a SARS-CoV-2 protein.

In some embodiments, the antigenic protein is a SARS-CoV-2 spike glycoprotein.

In some embodiments, the SARS-CoV-2 spike glycoprotein is a wild-type SARS-CoV-2 spike glycoprotein having an amino acid sequence of SEQ ID NO:123.

In some embodiments, the second polynucleotide comprises a sequence having at least 85% identity to a sequence of SEQ ID NO:121 or SEQ ID NO:122.

In some embodiments, the second polynucleotide comprises at least two transgenes.

In some embodiments, a second transgene encodes a second antigenic protein or a fragment thereof or an immunomodulatory protein.

In some embodiments, the second polynucleotide further comprises a sequence encoding a 2A peptide, an internal ribosomal entry site (IRES), or a combination thereof, located between transgenes.

In some embodiments, the immunomodulatory protein is a cytokine, a chemokine, or an interleukin.

In some embodiments, the second transgene encodes a second coronavirus protein.

In some embodiments, the first polynucleotide is located 5′ of the second polynucleotide.

In some embodiments, the nucleic acid molecule further comprises a second intergenic region located between the first polynucleotide and the second polynucleotide.

In some embodiments, the second intergenic region comprises a sequence having at least 85% identity to a sequence of SEQ ID NO:77.

In some embodiments, the nucleic acid molecule is
(a) a DNA molecule; or
(b) an RNA molecule, wherein T is substituted with U.

In some embodiments, the DNA molecule further comprises a promoter.

In some embodiments, the promoter is located 5′ of the 5′UTR.

In some embodiments, the promoter is a T7 promoter, a T3 promoter, or an SP6 promoter.

In some embodiments, the RNA molecule is a self-replicating RNA molecule.

In some embodiments, the RNA molecule further comprises a 5′ cap.

In some embodiments, the 5′ cap has a Cap 1 structure, a Cap 1 (^{m6}A) structure, a Cap 2 structure, a Cap 0 structure, or any combination thereof.

In yet another aspect, the disclosure provides a composition comprising any of the nucleic acid molecules provided herein. In some embodiments, the composition further comprises a lipid.

In some embodiments, the lipid comprises an ionizable cationic lipid.

In some embodiments, the ionizable cationic lipid has a structure of

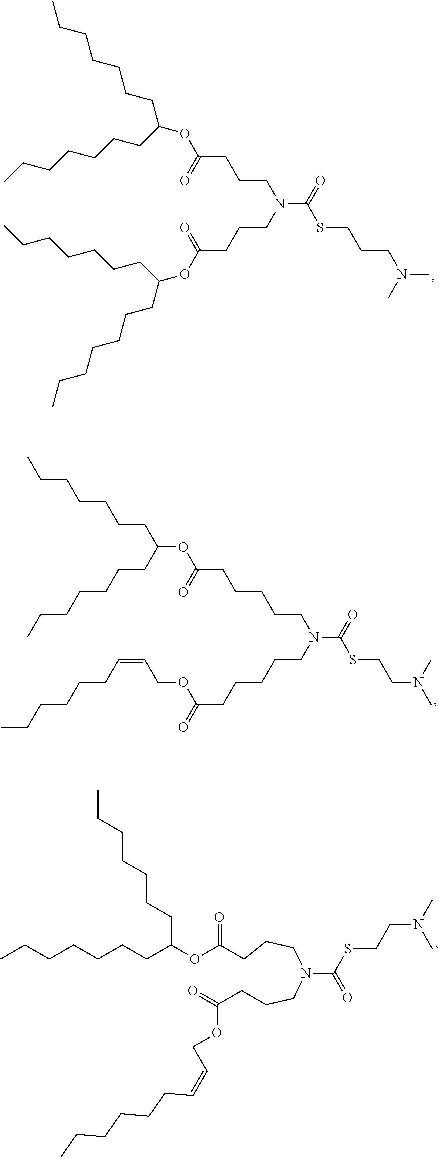

or a pharmaceutically acceptable salt thereof.

In yet another aspect, the disclosure provides a composition comprising any of the nucleic acid molecules described herein and a lipid formulation.

In some embodiments, the lipid formulation comprises an ionizable cationic lipid.

In some embodiments, the ionizable cationic lipid has a structure of

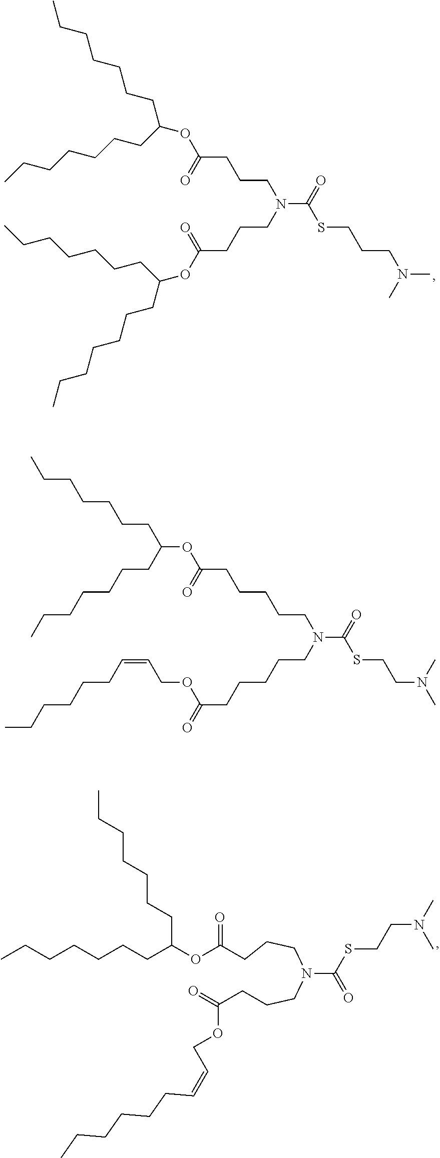

or a pharmaceutically acceptable salt thereof.

In some embodiments, the lipid formulation is selected from a lipoplex, a liposome, a lipid nanoparticle, a polymer-based carrier, an exosome, a lamellar body, a micelle, and an emulsion.

In some embodiments, the lipid formulation is a liposome selected from a cationic liposome, a nanoliposome, a proteoliposome, a unilamellar liposome, a multilamellar liposome, a ceramide-containing nanoliposome, and a multivesicular liposome.

In some embodiments, the lipid formulation is a lipid nanoparticle.

In some embodiments, the lipid nanoparticle has a size of less than about 200 nm. In some embodiments, the lipid nanoparticle has a size of less than about 150 nm. In some embodiments, the lipid nanoparticle has a size of less than about 100 nm. In some embodiments, the lipid nanoparticle has a size of about 55 nm to about 90 nm.

In some embodiments, the lipid formulation comprises one or more cationic lipids.

In some embodiments, the one or more cationic lipids is selected from 5-carboxyspermylglycinedioctadecylamide (DOGS), 2,3-dioleyloxy-N-[2(spermine-carboxamido)ethyl]-N,N-dimethyl-1-propanaminium (DOSPA), 1,2-Dioleoyl-3-Dimethylammonium-Propane (DODAP), 1,2-Dioleoyl-3-Trimethylammonium-Propane (DOTAP), 1,2-distearyloxy-N,N-dimethyl-3-aminopropane (DSDMA), 1,2-dioleyloxy-N,N-dimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA), N-dioleyl-N,N-dimethylammonium chloride (DODAC), N,N-distearyl-N,N-dimethylammonium bromide (DDAB), N-(1,2-dimyristyloxyprop-3-yl)-N,N-dimethyl-N-hydroxyethyl ammonium bromide (DMRIE), 3-dimethylamino-2-(cholest-5-en-3-beta-oxybutan-4-oxy)-1-(cis,cis-9,12-oc-tadecadienoxy)propane (CLinDMA), 2-[5′-(cholest-5-en-3-beta-oxy)-3′-oxapentoxy)-3-dimethy 1-1-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), and 2,2-dilinoleyl-4-dimethylaminoethyl-[1,3]-dioxolane or (DLin-K-XTC2-DMA).

In some embodiments, the lipid formulation comprises an ionizable cationic lipid.

In some embodiments, the ionizable cationic lipid has a structure of Formula I:

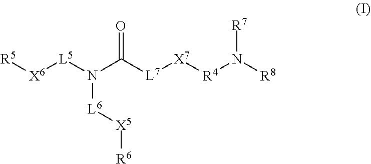

or a pharmaceutically acceptable salt or solvate thereof, wherein R⁵ and R⁶ are each independently selected from the group consisting of a linear or branched C₁-C₃₁ alkyl, C₂-C₃₁ alkenyl or C₂-C₃₁ alkynyl and cholesteryl; L⁵ and L⁶ are each independently selected from the group consisting of a linear C₁-C₂₀ alkyl and C₂-C₂₀ alkenyl; X⁵ is —C(O)O—, whereby —C(O)O—R⁶ is formed or —OC(O)— whereby —OC(O)—R⁶ is formed; X⁶ is —C(O)O— whereby —C(O)O—R⁵ is formed or —OC(O)— whereby —OC(O)—R⁵ is formed; X⁷ is S or O; L⁷ is absent or lower alkyl; R⁴ is a linear or branched C₁-C₆ alkyl; and R⁷ and R⁸ are each independently selected from the group consisting of a hydrogen and a linear or branched C₁-C₆ alkyl.

In some embodiments, the ionizable cationic lipid is selected from

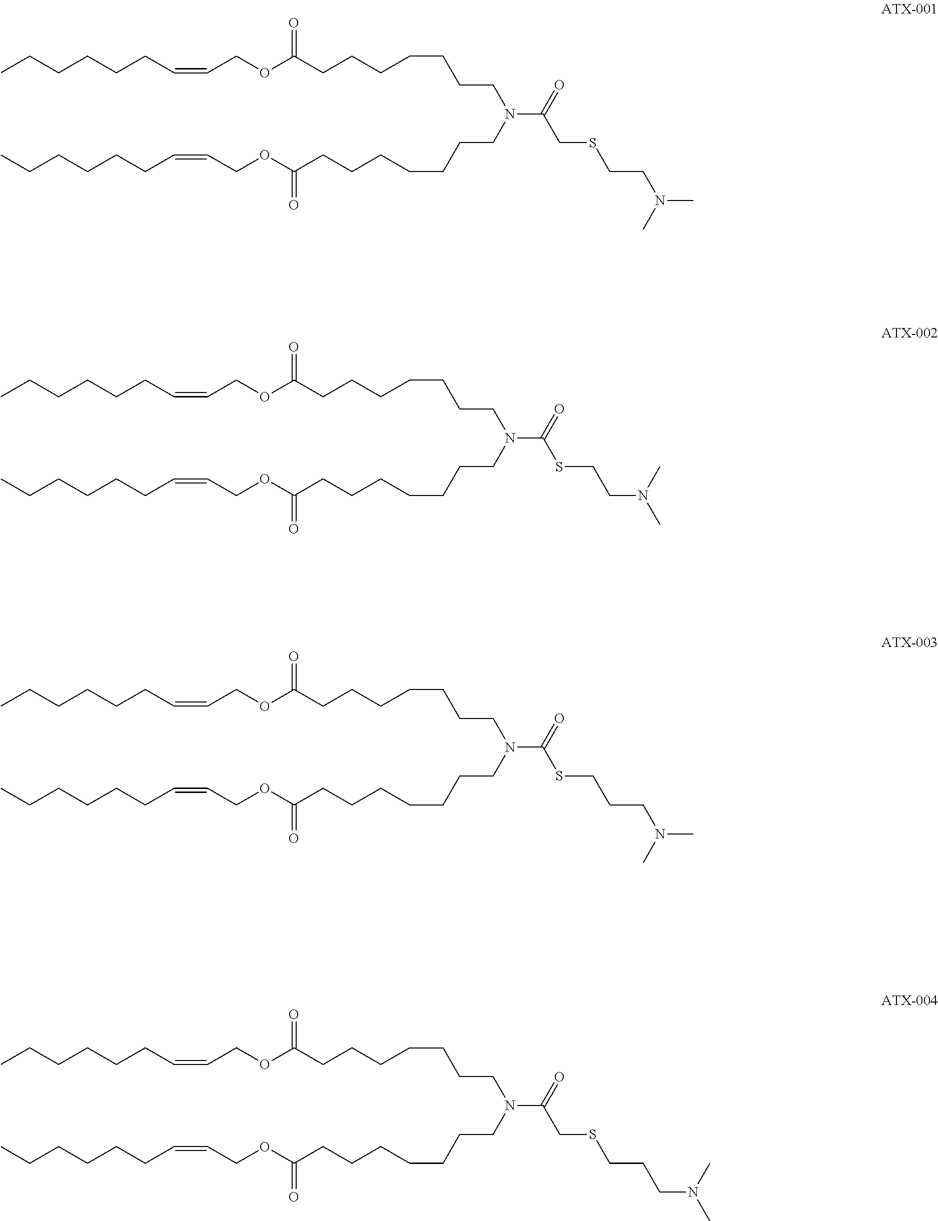

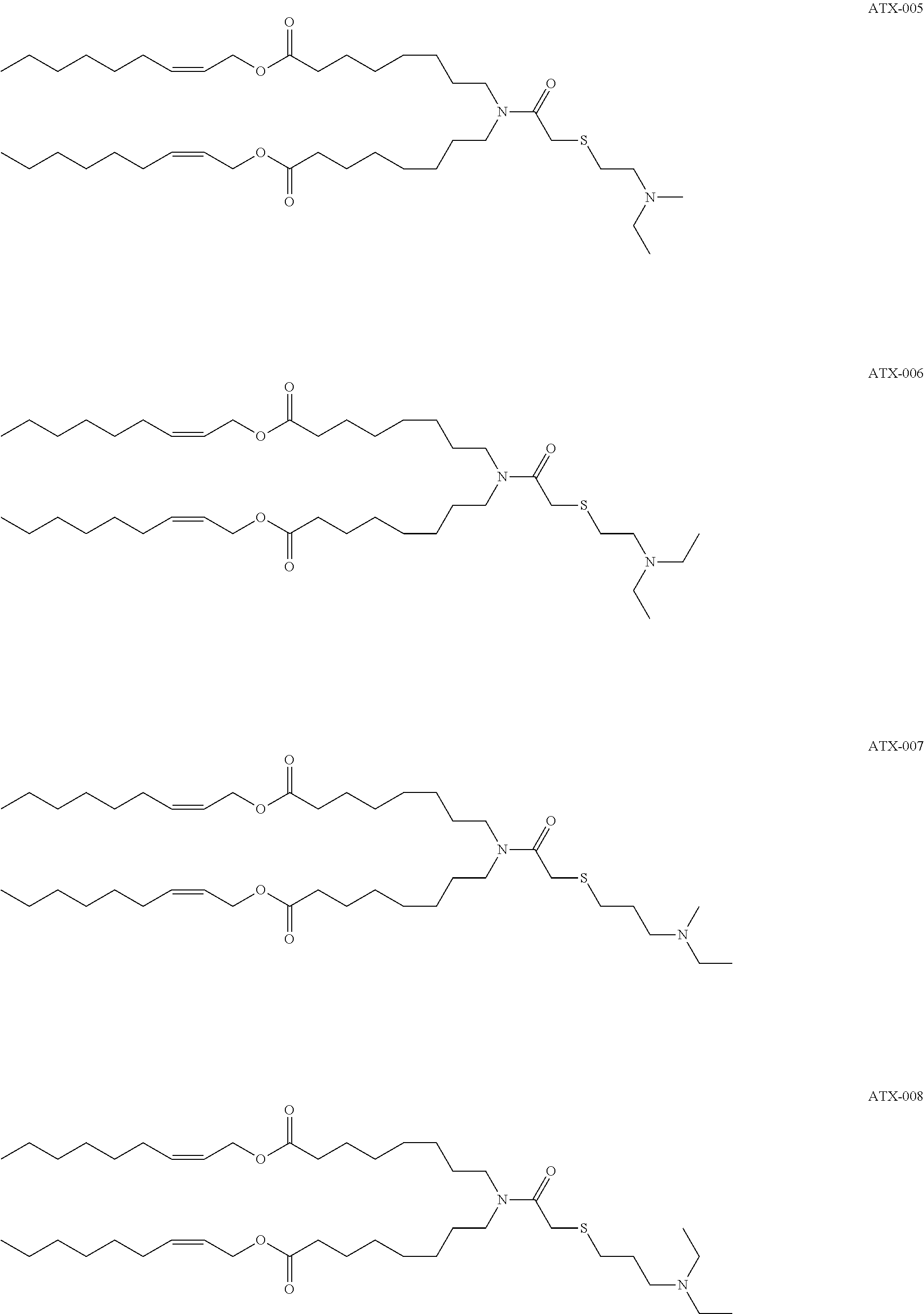

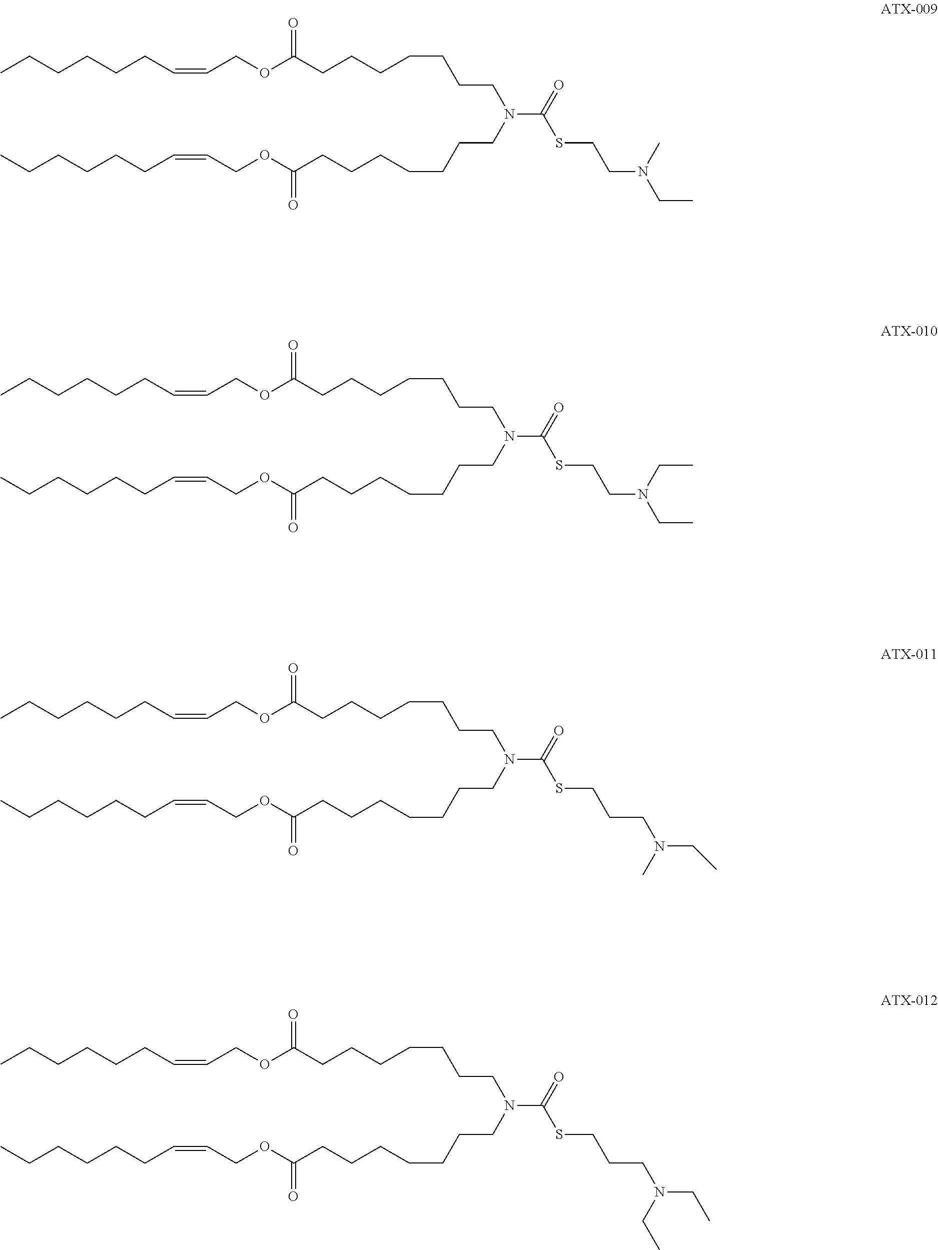

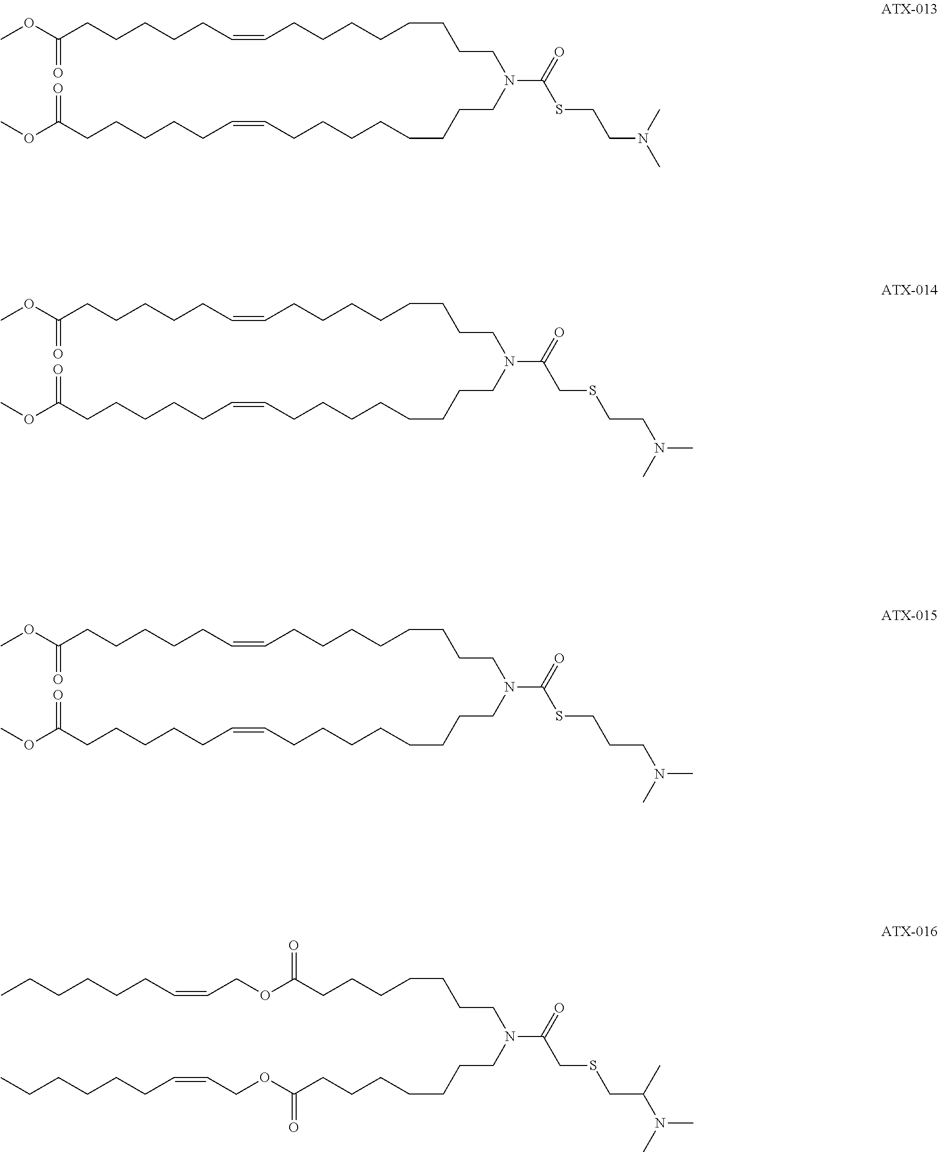

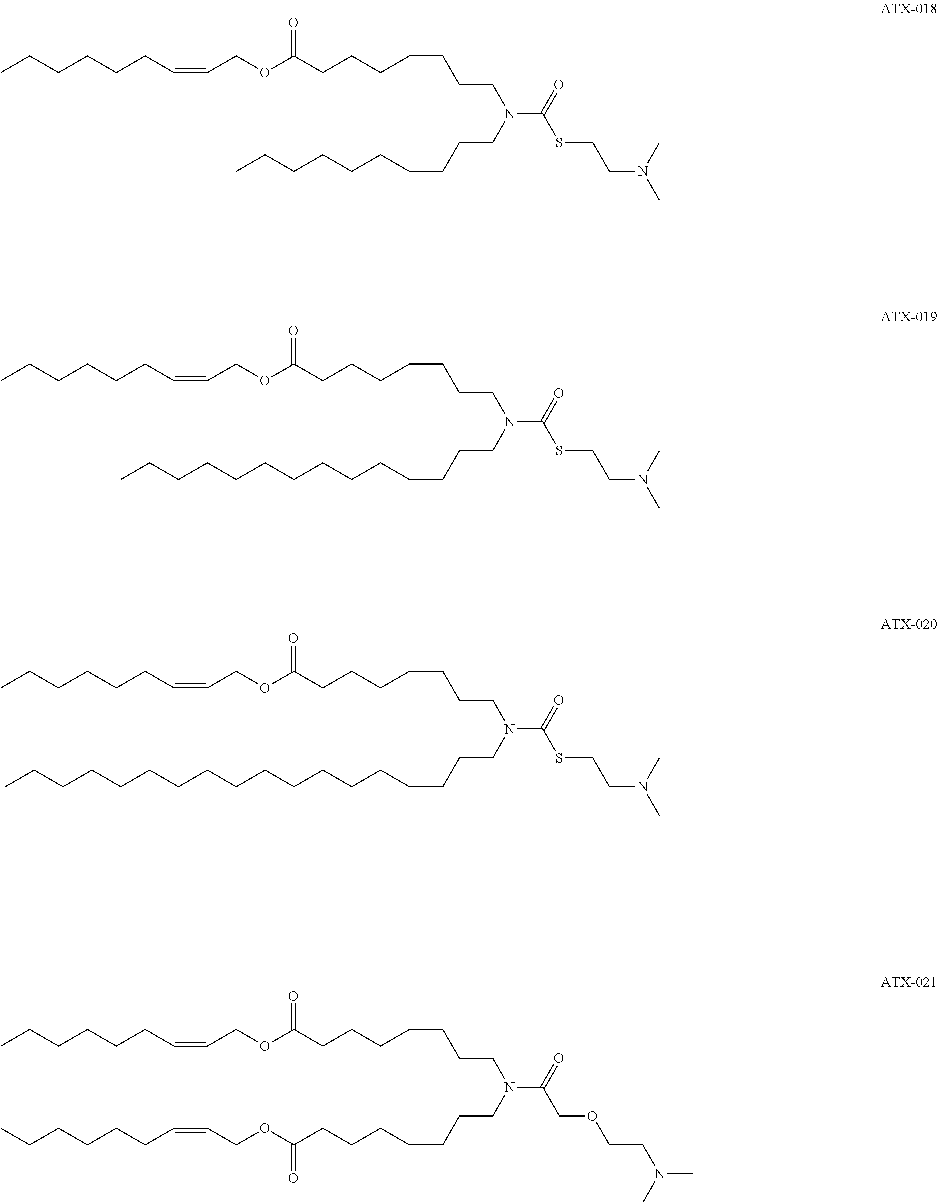

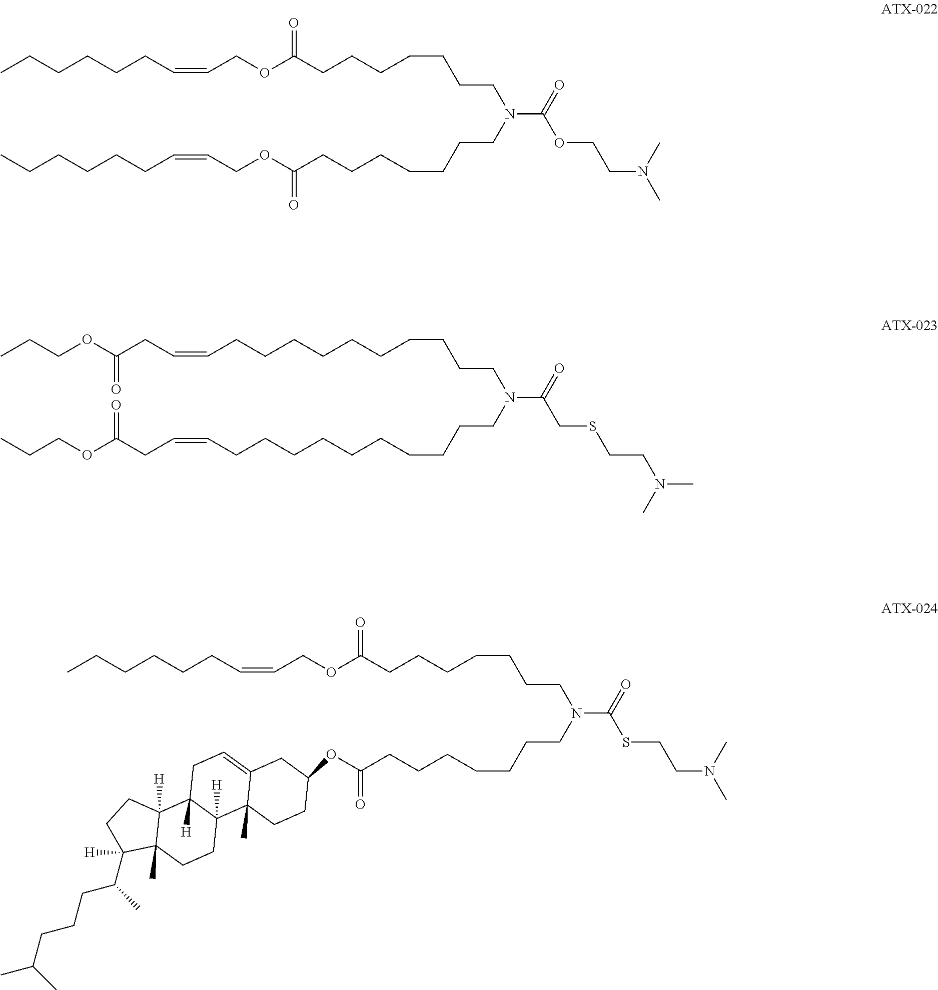

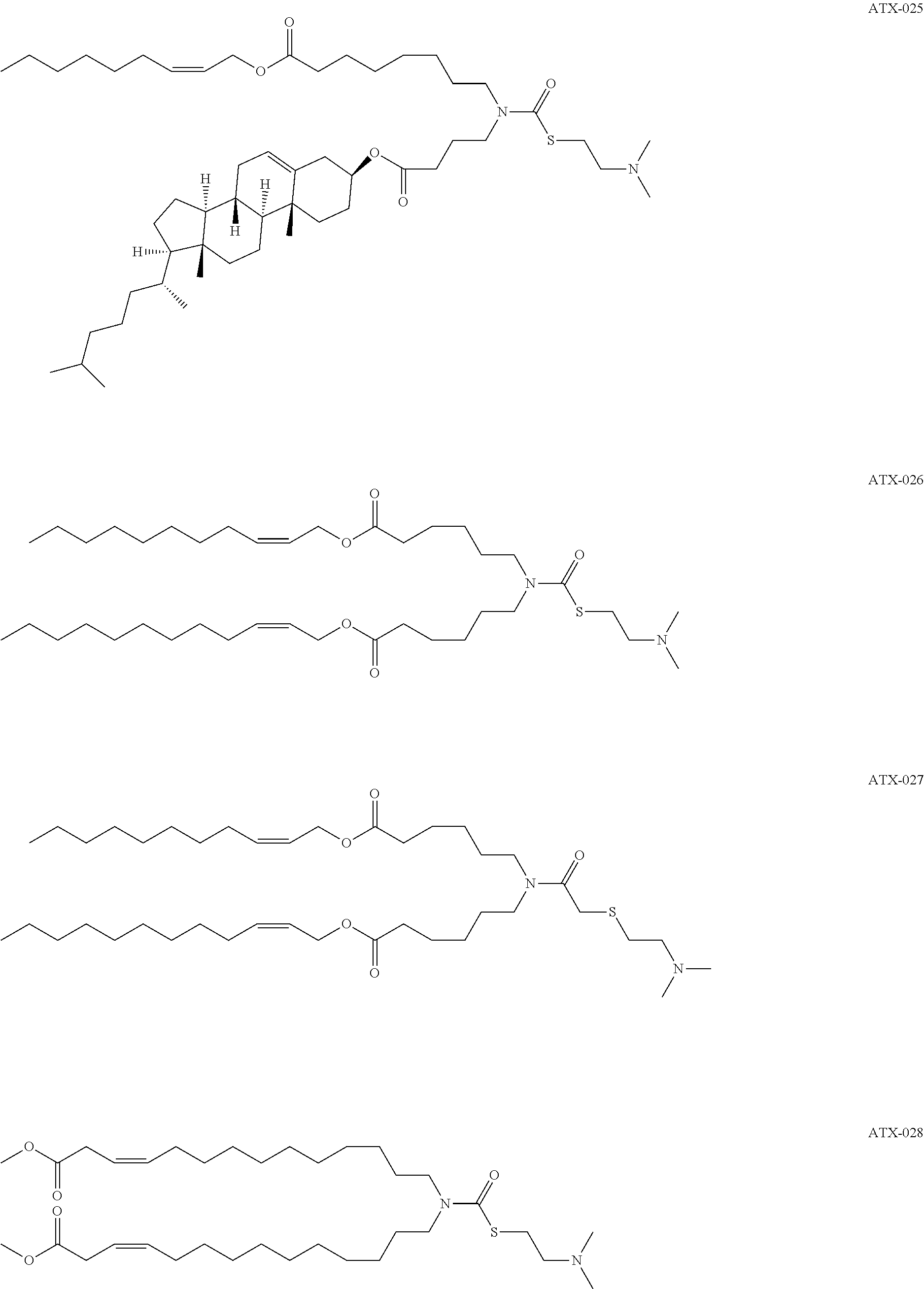

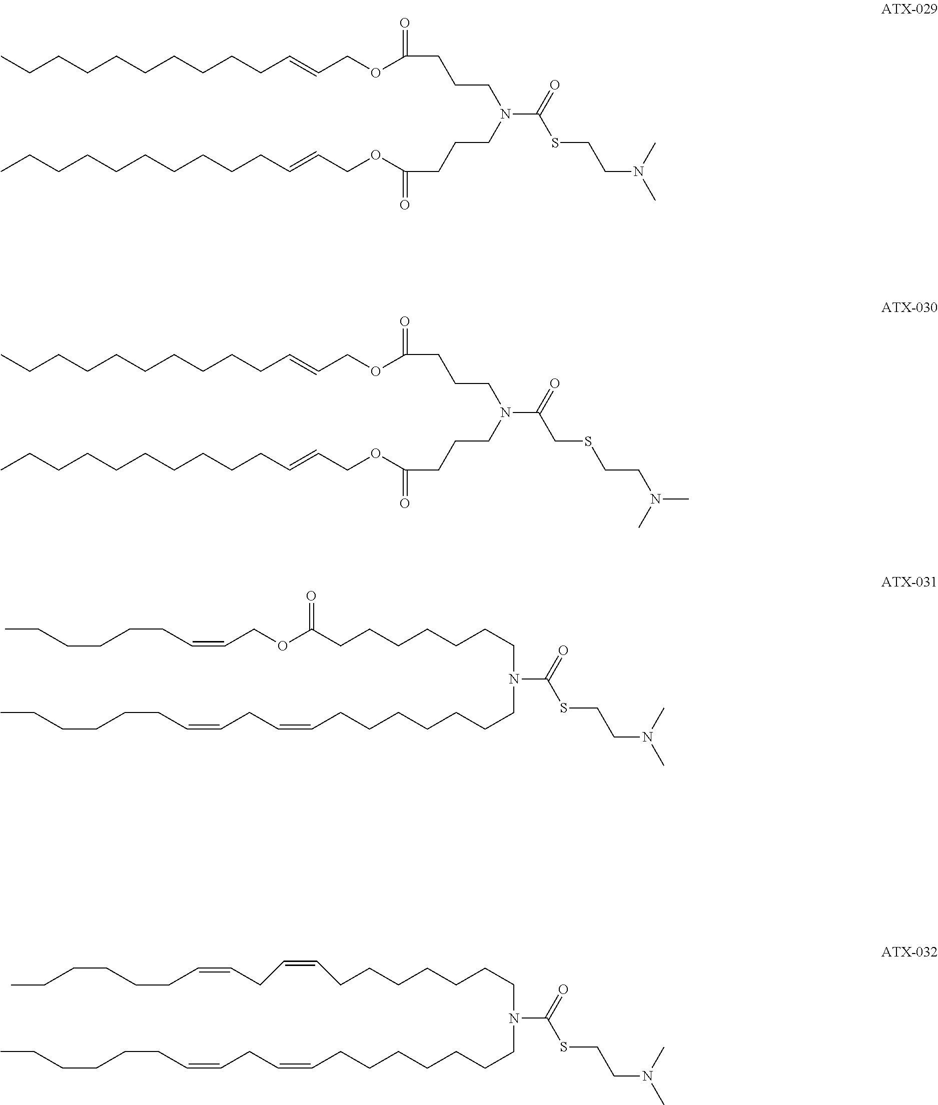

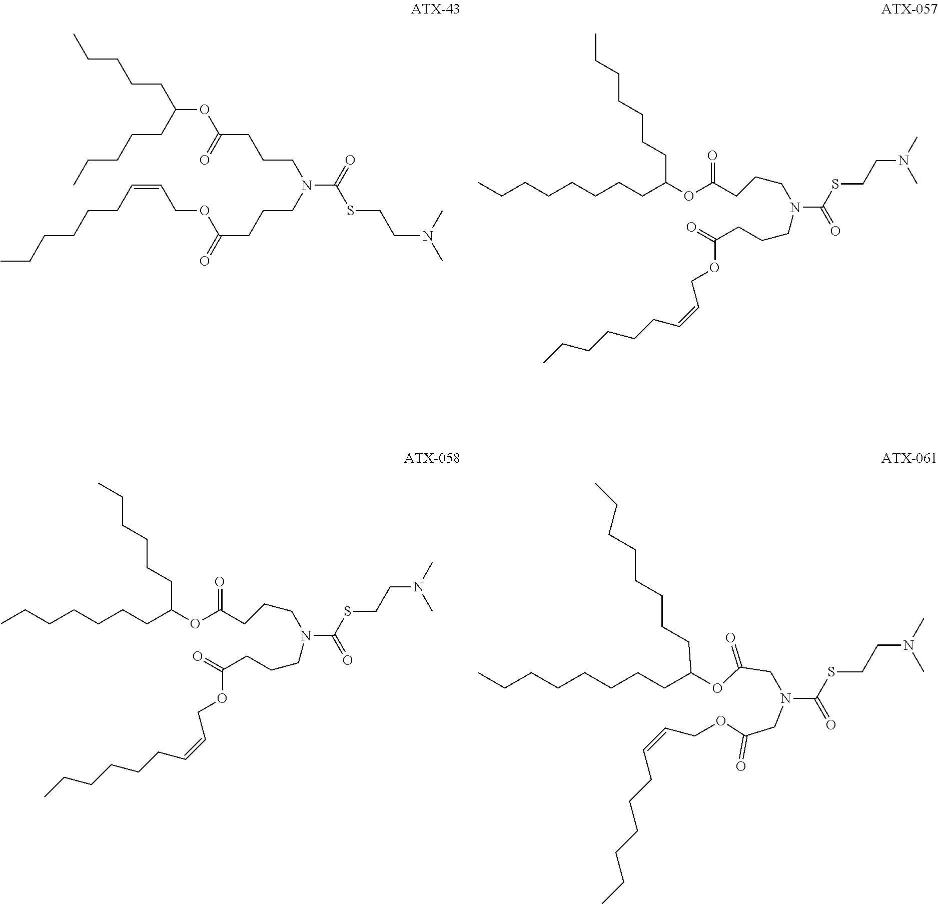 [SMILES: CCCCCC/C=C\COC(=O)CCCN(CCCC(=O)OC(CCCCC)CCCCC)C(=O)SCCN(C)C ; CCCCCC/C=C\COC(=O)CCCN(CCCC(=O)OC(CCCCCCC)CCCCCCCC)C(=O)SCCN(C)C ; CCCCCC/C=C\COC(=O)CCCN(CCCC(=O)OC(CCCCCC)CCCCCCC)C(=O)SCCN(C)C ; CCCCCC/C=C\COC(=O)CN(CC(=O)OC(CCCCCCCC)CCCCCCCC)C(=O)SCCN(C)C]

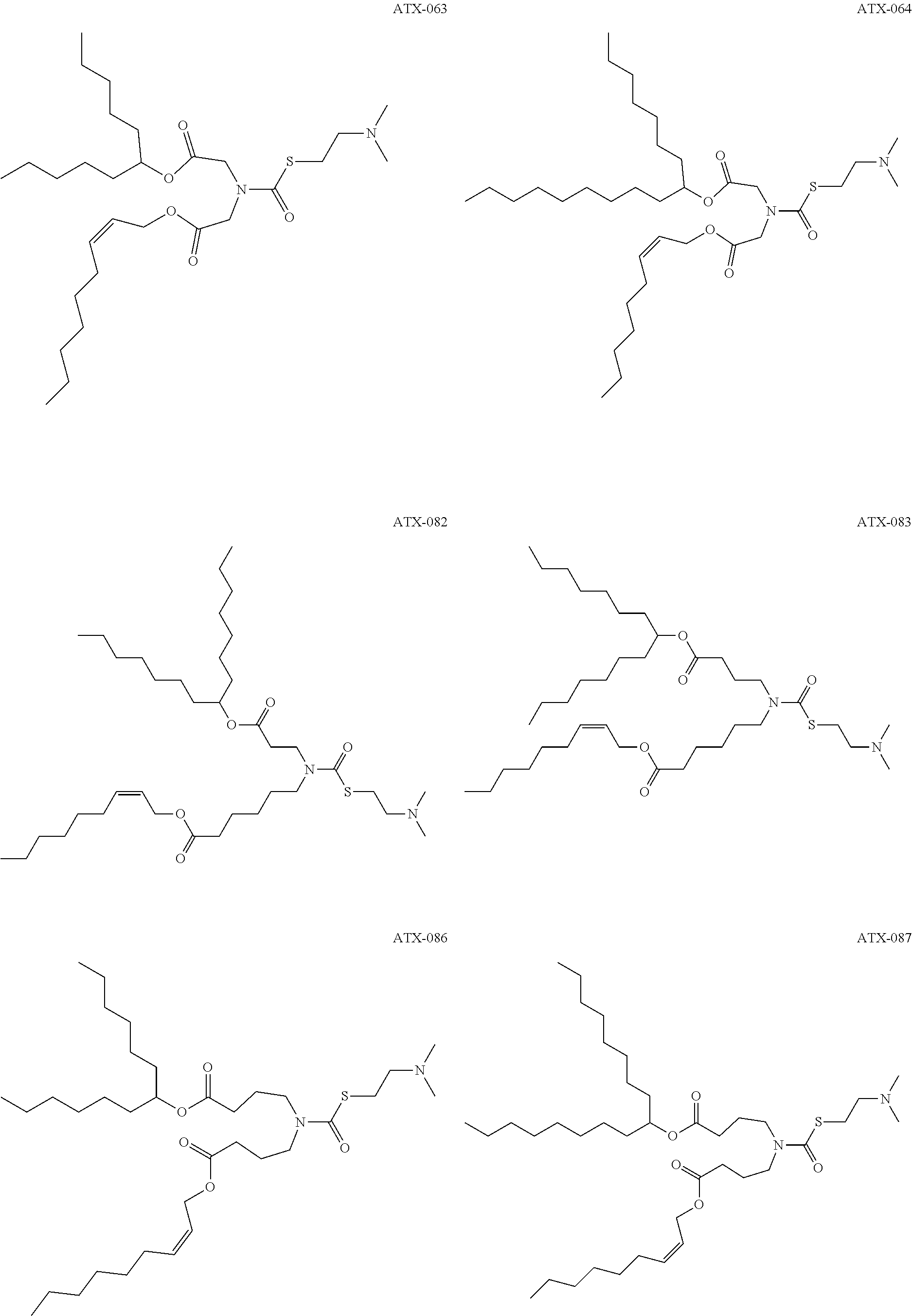 [SMILES: CCCCCC/C=C\COC(=O)CN(CC(=O)OC(CCCCC)CCCCC)C(=O)SCCN(C)C ; CCCCCC/C=C\COC(=O)CN(CC(=O)OC(CCCCCCC)CCCCCCCCC)C(=O)SCCN(C)C ; CCCCCC/C=C\COC(=O)CCCCCN(CCC(=O)OC(CCCCCCC)CCCCCCC)C(=O)SCCN(C)C ; CCCCCC/C=C\COC(=O)CCCCCN(CCCC(=O)OC(CCCCCCC)CCCCCCC)C(=O)SCCN(C)C ; CCCCCC/C=C\COC(=O)CCCN(CCCC(=O)OC(CCCCCC)CCCCCC)C(=O)SCCN(C)C ; CCCCCC/C=C\COC(=O)CCCN(CCCC(=O)OC(CCCCCCCC)CCCCCCCC)C(=O)SCCN(C)C]

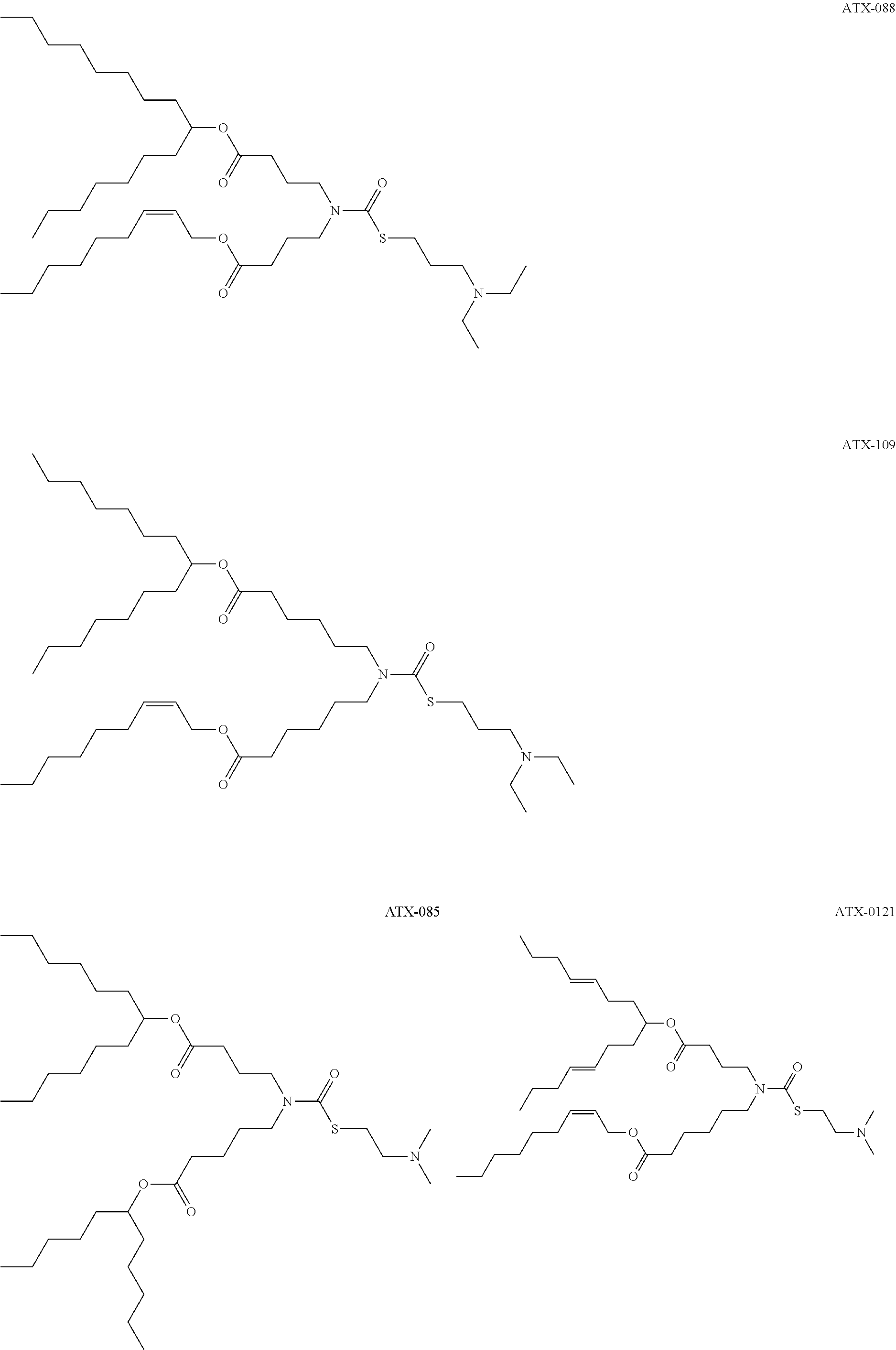

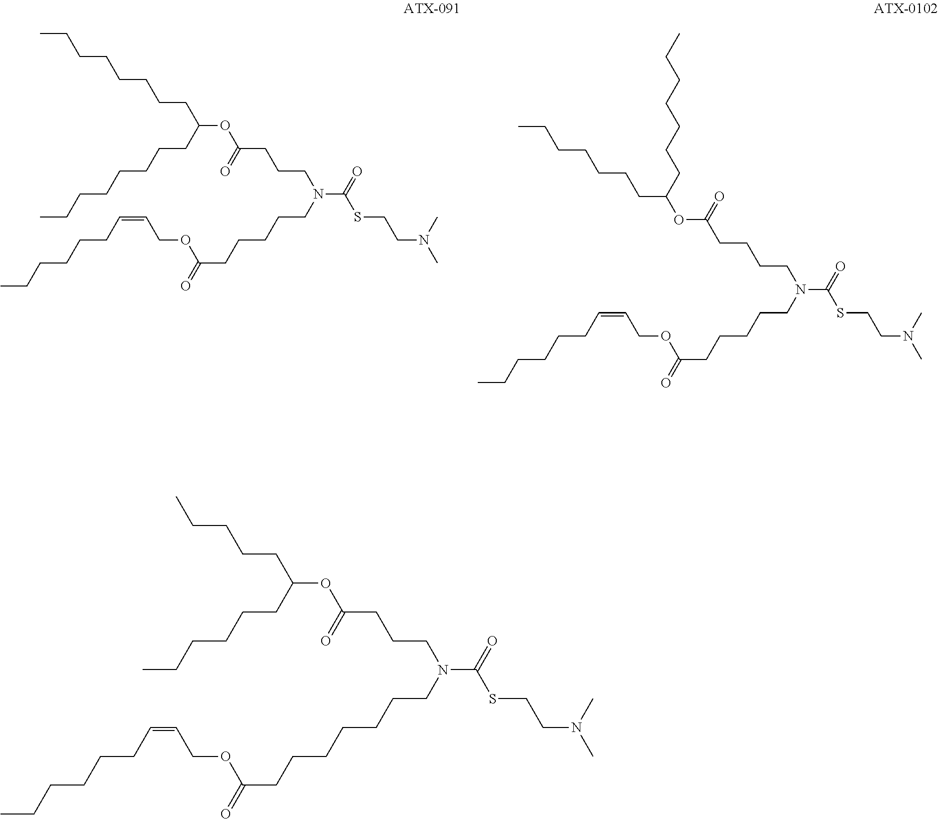 [SMILES: CCCCCC/C=C\COC(=O)CCCCCN(CCCC(=O)OC(CCCCCCCC)CCCCCCCC)C(=O)SCCN(C)C ; CCCCCC/C=C\COC(=O)CCCCCN(CCCCC(=O)OC(CCCCCCC)CCCCCCC)C(=O)SCCN(C)C ; CCCCCC/C=C\COC(=O)CCCCCCCN(CCCC(=O)OC(CCCCC)CCCCCC)C(=O)SCCN(C)C]

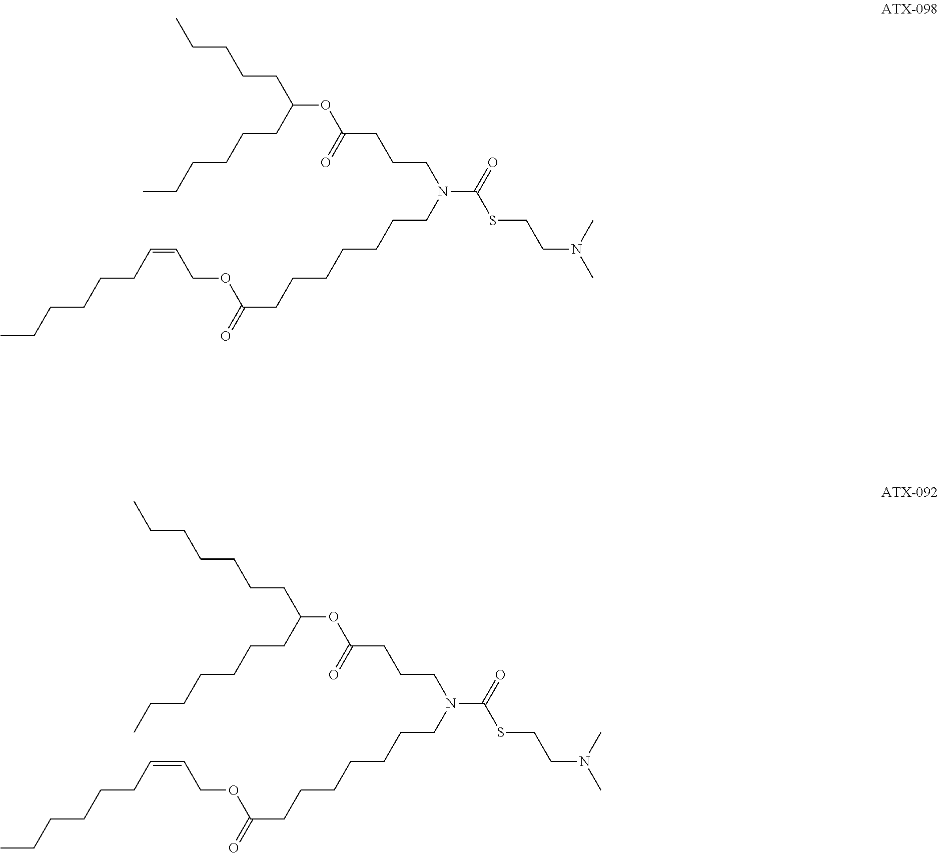

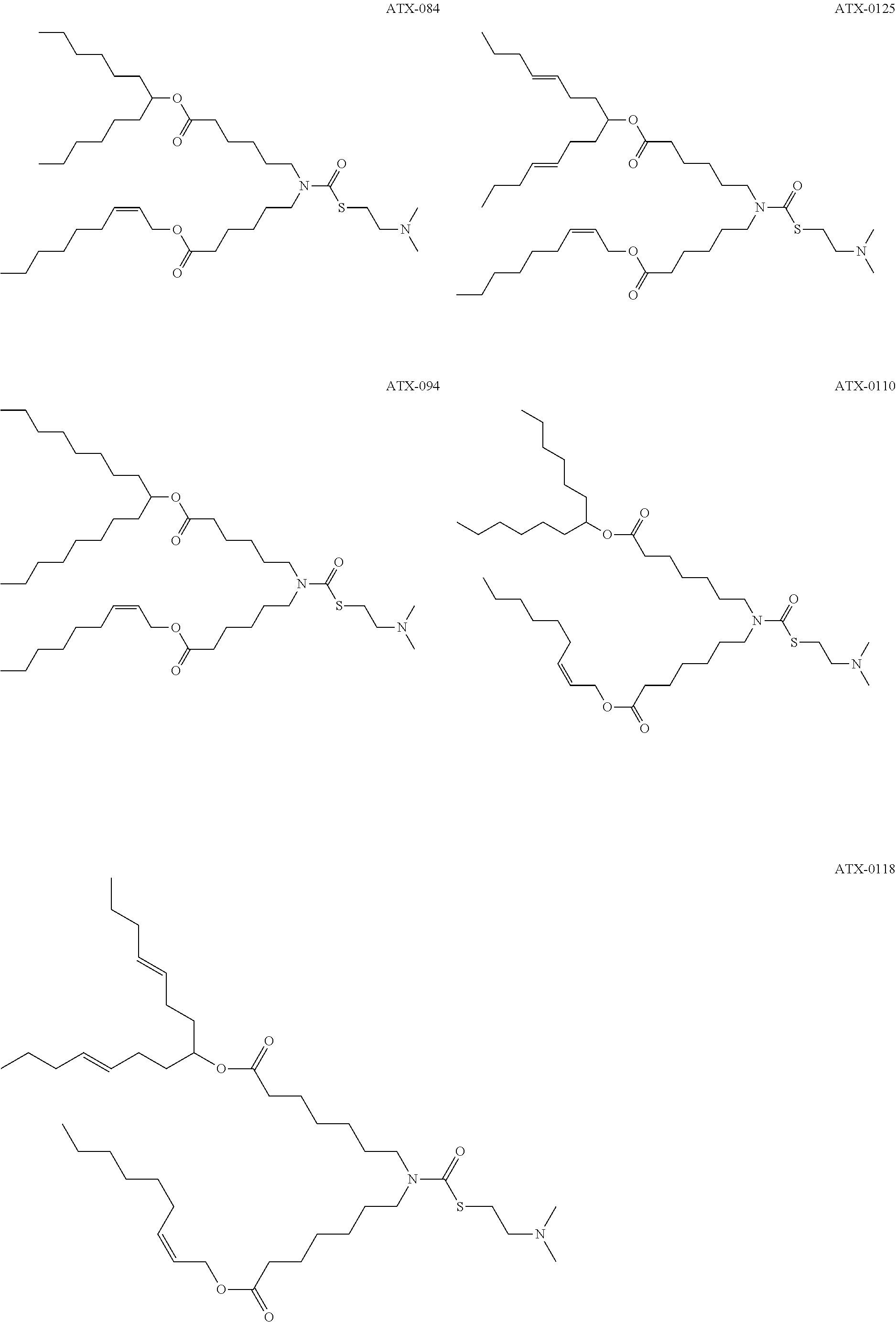 [SMILES: CCCCCC/C=C\COC(=O)CCCCCN(CCCCCC(=O)OC(CCCCCC)CCCCCC)C(=O)SCCN(C)C ; CCC/C=C/CCC(CC/C=C/CCC)OC(=O)CCCCCN(CCCCCC(=O)OC/C=C\CCCCCC)C(=O)SCCN(C)C ; CCCCCC/C=C\COC(=O)CCCCCN(CCCCCC(=O)OC(CCCCCCCC)CCCCCCCC)C(=O)SCCN(C)C ; CCCCCC/C=C\COC(=O)CCCCCCN(CCCCCCC(=O)OC(CCCCCC)CCCCCC)C(=O)SCCN(C)C ; CCC/C=C/CCC(CC/C=C/CCC)OC(=O)CCCCCCN(CCCCCCC(=O)OC/C=C\CCCCCC)C(=O)SCCN(C)C]

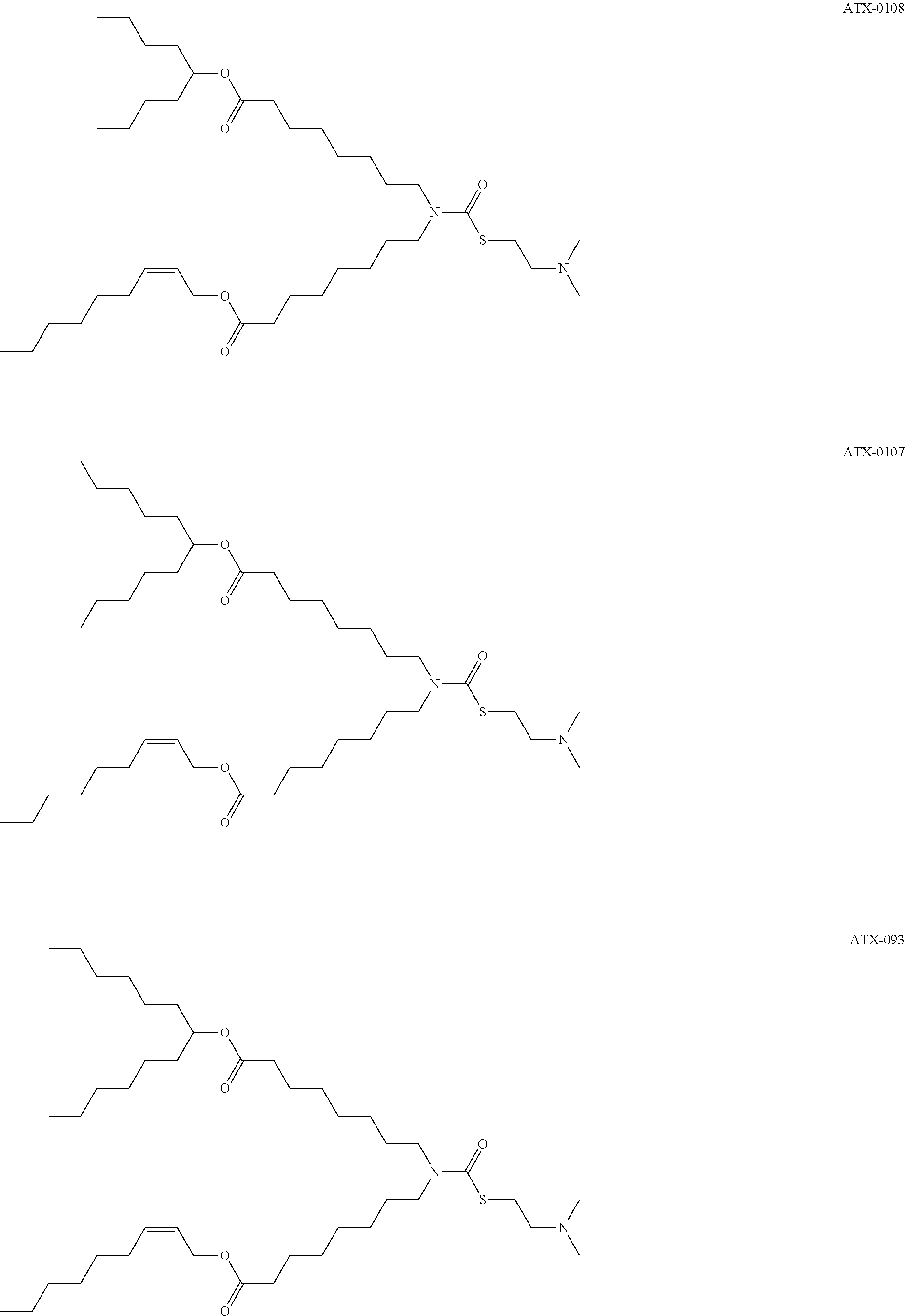

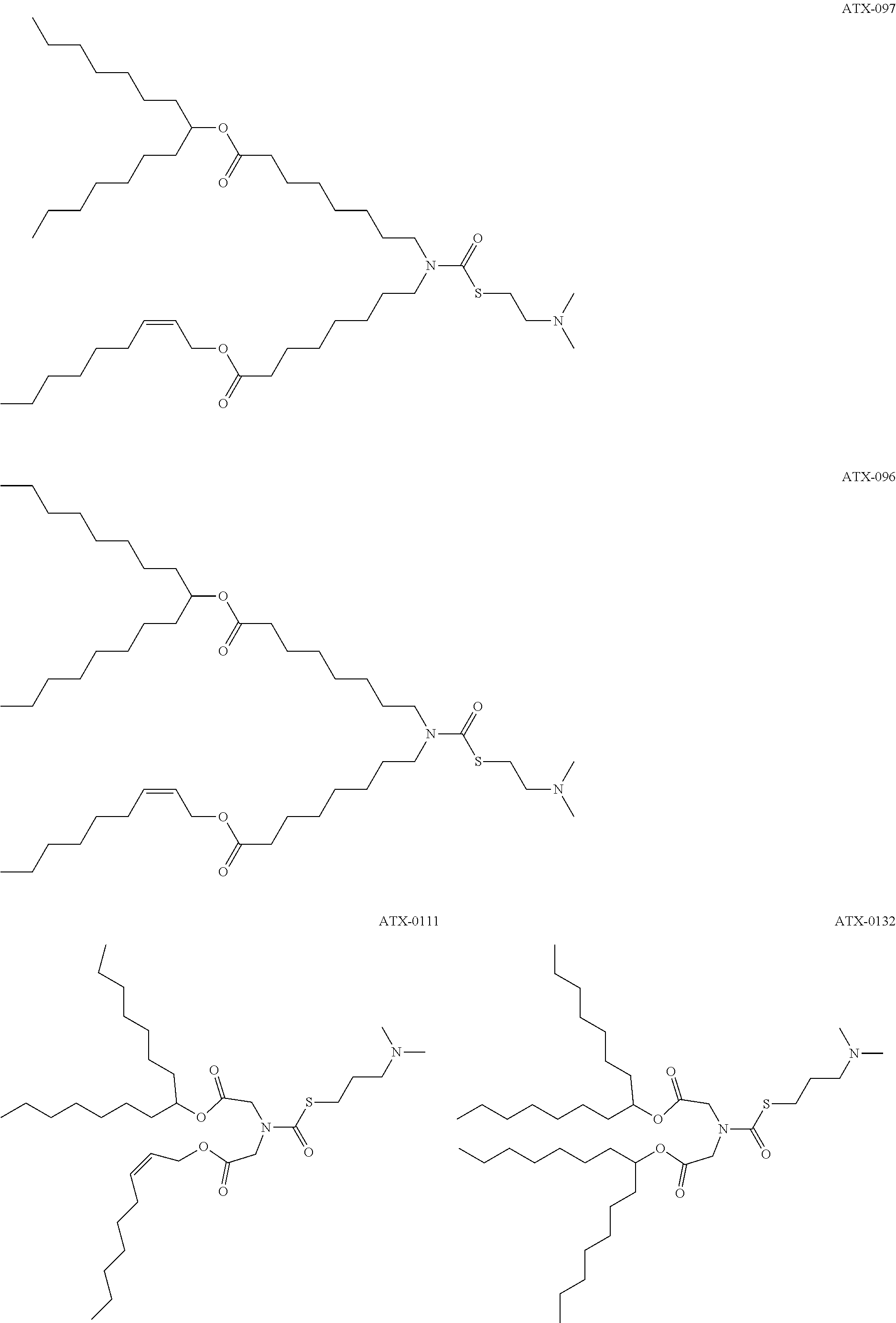

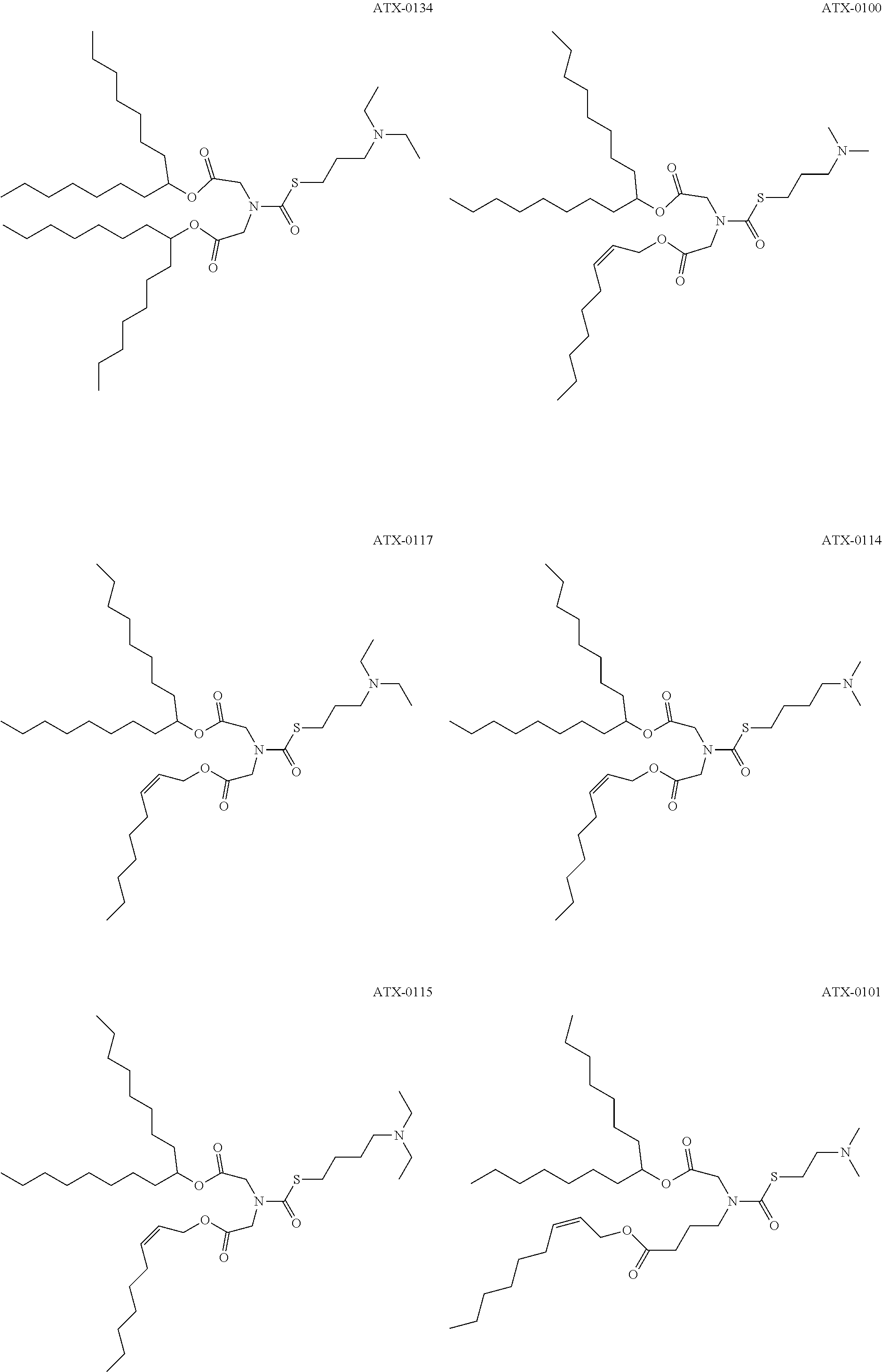 [SMILES: CCCCCCCC(CCCCCCC)OC(=O)CN(CC(=O)OC(CCCCCCC)CCCCCCC)C(=O)SCCCN(CC)CC ; CCCCCC/C=C\COC(=O)CN(CC(=O)OC(CCCCCCCC)CCCCCCCC)C(=O)SCCCN(C)C ; CCCCCC/C=C\COC(=O)CN(CC(=O)OC(CCCCCCCC)CCCCCCCC)C(=O)SCCCN(CC)CC ; CCCCCC/C=C\COC(=O)CN(CC(=O)OC(CCCCCCCC)CCCCCCCC)C(=O)SCCCCN(C)C ; CCCCCC/C=C\COC(=O)CN(CC(=O)OC(CCCCCCCC)CCCCCCCC)C(=O)SCCCCN(CC)CC ; CCCCCC/C=C\COC(=O)CCCN(CC(=O)OC(CCCCCCC)CCCCCCC)C(=O)SCCN(C)C]

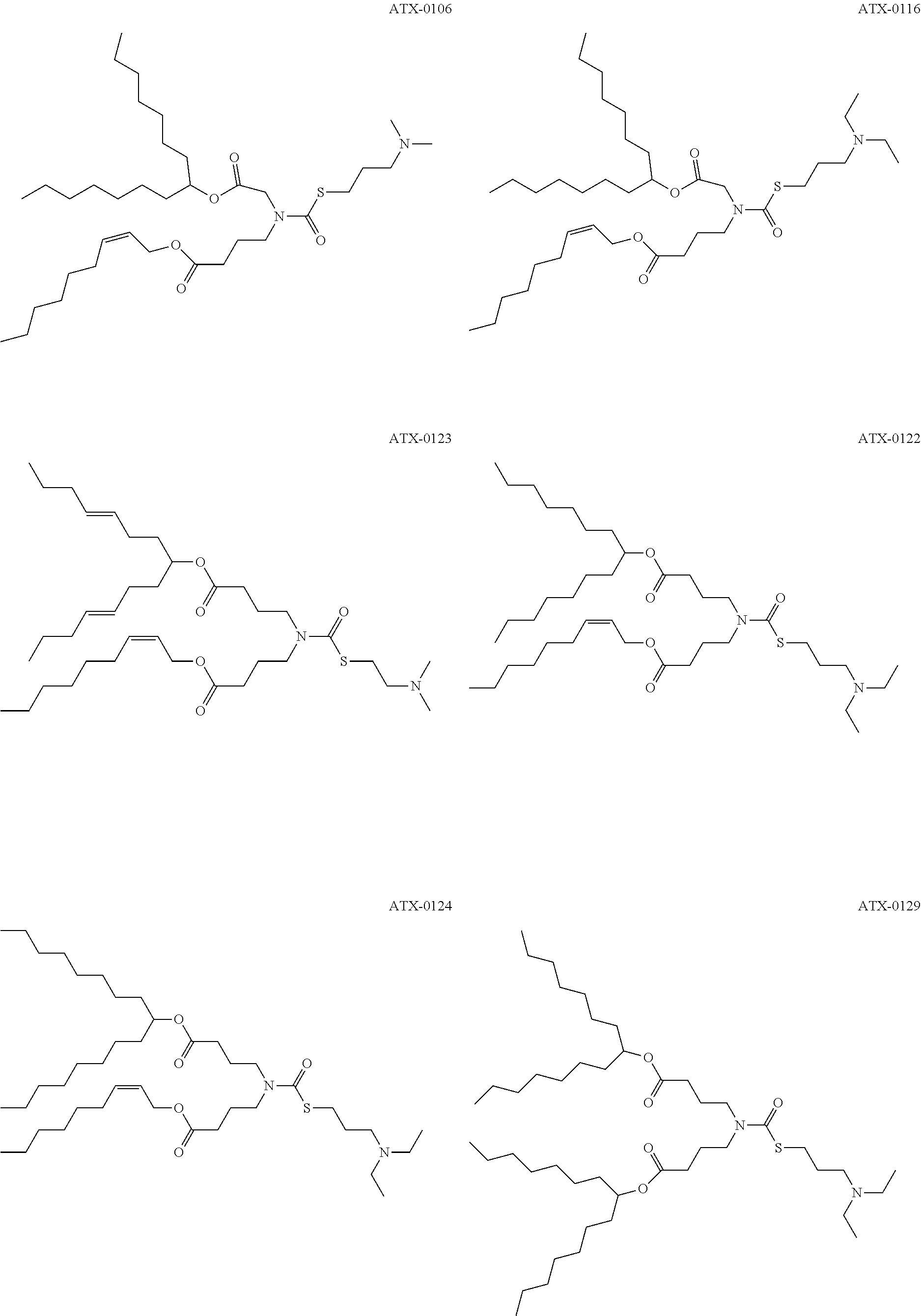 [SMILES: CCCCCC/C=C\COC(=O)CCCN(CC(=O)OC(CCCCCCC)CCCCCCC)C(=O)SCCCN(C)C ; CCCCCC/C=C\COC(=O)CCCN(CC(=O)OC(CCCCCCC)CCCCCCC)C(=O)SCCCN(CC)CC ; CCC/C=C/CCC(CC/C=C/CCC)OC(=O)CCCN(CCCC(=O)OC/C=C\CCCCCC)C(=O)SCCN(C)C ; CCCCCC/C=C\COC(=O)CCCN(CCCC(=O)OC(CCCCCCC)CCCCCCC)C(=O)SCCCN(CC)CC ; CCCCCC/C=C\COC(=O)CCCN(CCCC(=O)OC(CCCCCCCC)CCCCCCCC)C(=O)SCCCN(CC)CC ; CCCCCCCC(CCCCCCC)OC(=O)CCCN(CCCC(=O)OC(CCCCCCC)CCCCCCC)C(=O)SCCCN(CC)CC]

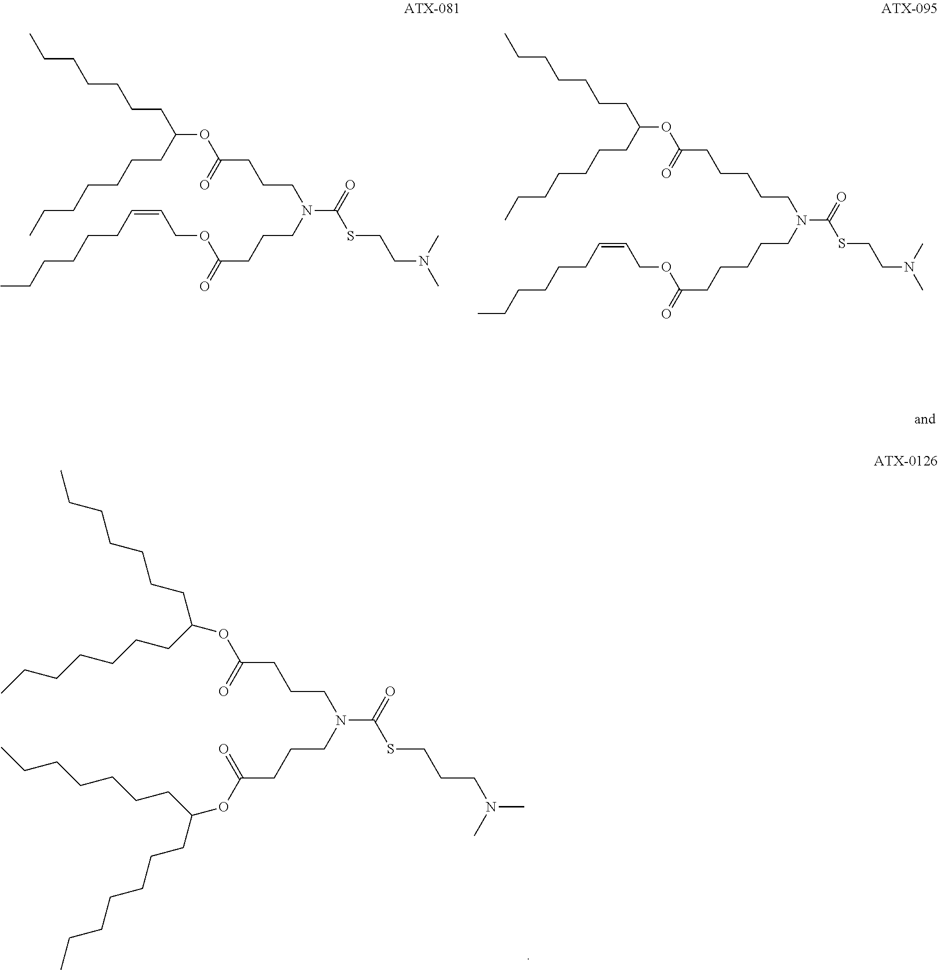 [SMILES: CCCCCC/C=C\COC(=O)CCCN(CCCC(=O)OC(CCCCCCC)CCCCCCC)C(=O)SCCN(C)C ; CCCCCC/C=C\COC(=O)CCCCCN(CCCCCC(=O)OC(CCCCCCC)CCCCCCC)C(=O)SCCN(C)C ; CCCCCCCC(CCCCCCC)OC(=O)CCCN(CCCC(=O)OC(CCCCCCC)CCCCCCC)C(=O)SCCCN(C)C]

In some embodiments, the ionizable cationic lipid is ATX-126:

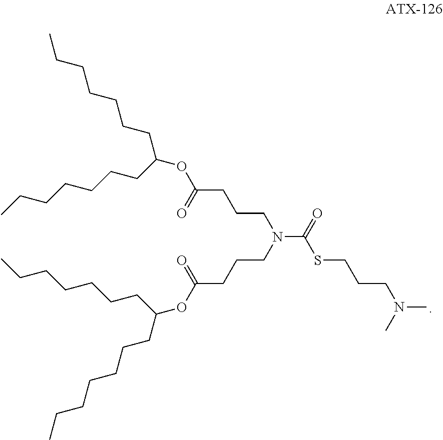 [SMILES: CCCCCCCC(CCCCCCC)OC(=O)CCCN(CCCC(=O)OC(CCCCCCC)CCCCCCC)C(=O)SCCCN(C)C]

In some embodiments, the lipid formulation encapsulates the nucleic acid molecule.

In some embodiments, the lipid formulation is complexed to the nucleic acid molecule.

In some embodiments, the lipid formulation further comprises a helper lipid. In some embodiments, the helper lipid is a phospholipid. In some embodiments, the helper lipid is selected from dioleoylphosphatidyl ethanolamine (DOPE), dimyristoylphosphatidyl choline (DMPC), distearoylphosphatidyl choline (DSPC), dimyristoylphosphatidyl glycerol (DMPG), dipalmitoyl phosphatidylcholine (DPPC), and phosphatidylcholine (PC). In specific embodiments, the helper lipid is distearoylphosphatidylcholine (DSPC).

In some embodiments, the lipid formulation further comprises cholesterol.

In some embodiments, the lipid formulation further comprises a polyethylene glycol (PEG)-lipid conjugate. In some embodiments, the PEG-lipid conjugate is PEG-DMG. In some embodiments, the PEG-DMG is PEG2000-DMG.

In some embodiments, the lipid portion of the lipid formulation comprises about 40 mol % to about 60 mol % of the ionizable cationic lipid, about 4 mol % to about 16 mol % DSPC, about 30 mol % to about 47 mol % cholesterol, and about 0.5 mol % to about 3 mol % PEG2000-DMG.

In some embodiments, the lipid portion of the lipid formulation comprises about 42 mol % to about 58 mol % of the ionizable cationic lipid, about 6 mol % to about 14 mol % DSPC, about 32 mol % to about 44 mol % cholesterol, and about 1 mol % to about 2 mol % PEG2000-DMG.

In some embodiments, the lipid portion of the lipid formulation comprises about 45 mol % to about 55 mol % of the ionizable cationic lipid, about 8 mol % to about 12 mol % DSPC, about 35 mol % to about 42 mol % cholesterol, and about 1.25 mol % to about 1.75 mol % PEG2000-DMG.

In some embodiments, the composition has a total lipid:nucleic acid molecule weight ratio of about 50:1 to about 10:1. In some embodiments, the composition has a total lipid:nucleic acid molecule weight ratio of about 44:1 to about 24:1. In some embodiments, the composition has a total lipid:nucleic acid molecule weight ratio of about 40:1 to about 28:1. In some embodiments, the composition has a total lipid:nucleic acid molecule weight ratio of about 38:1 to about 30:1. In some embodiments, the composition has a total lipid:nucleic acid molecule weight ratio of about 37:1 to about 33:1. In some embodiments, the composition comprises a HEPES or TRIS buffer at a pH of about 7.0 to about 8.5.

In some embodiments, the HEPES or TRIS buffer is at a concentration of about 7 mg/mL to about 15 mg/mL.

In some embodiments, the composition further comprises about 2.0 mg/mL to about 4.0 mg/mL of NaCl.

In some embodiments, the composition further comprises one or more cryoprotectants.

In some embodiments, the one or more cryoprotectants are selected from sucrose, glycerol, or a combination of sucrose and glycerol.

In some embodiments, the composition comprises a combination of sucrose at a concentration of about 70 mg/mL to about 110 mg/mL of sucrose and glycerol at a concentration of about 50 mg/mL to about 70 mg/mL.

In some embodiments, the composition is a lyophilized composition.

In some embodiments, the lyophilized composition comprises one or more lyoprotectants.

In some embodiments, the lyophilized composition comprises a poloxamer, potassium sorbate, sucrose, or any combination thereof.

In some embodiments, the poloxamer is poloxamer 188.

In some embodiments, the lyophilized composition comprises about 0.01 to about 1.0% w/w of the nucleic acid molecule.

In some embodiments, the lyophilized composition comprises about 1.0 to about 5.0% w/w lipids.

In some embodiments, the lyophilized composition comprises about 0.5 to about 2.5% w/w of TRIS buffer.

In some embodiments, the lyophilized composition comprises about 0.75 to about 2.75% w/w of NaCl.

In some embodiments, the lyophilized composition comprises about 85 to about 95% w/w of a sugar. In some embodiments, the sugar is sucrose.

In some embodiments, the lyophilized composition comprises about 0.01 to about 1.0% w/w of a poloxamer. In some embodiments, the poloxamer is poloxamer 188.

In some embodiments, the lyophilized composition comprises about 1.0 to about 5.0% w/w of potassium sorbate.

In some embodiments, the nucleic acid molecule comprises
(a) a sequence of SEQ ID NO:124;
(b) a sequence of SEQ ID NO:124, wherein T is substituted with U;
(c) a sequence of SEQ ID NO:125; or
(d) a sequence of SEQ ID NO:125, wherein T is substituted with U.

In yet another aspect, the disclosure provides a lipid nanoparticle composition comprising
a. a lipid formulation comprising
   i. about 45 mol % to about 55 mol % of an ionizable cationic lipid having the structure of ATX-126:

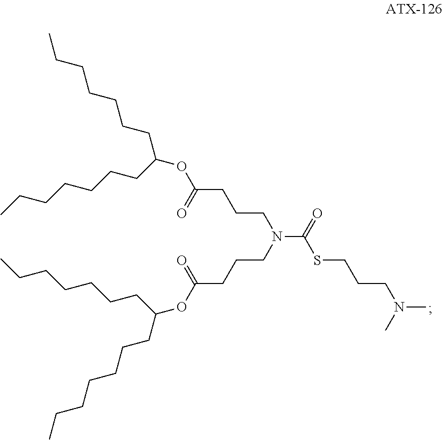 [SMILES: CCCCCCCC(CCCCCCC)OC(=O)CCCN(CCCC(=O)OC(CCCCCCC)CCCCCCC)C(=O)SCCCN(C)C]

ii. about 8 mol % to about 12 mol % DSPC;
iii. about 35 mol % to about 42 mol % cholesterol; and
iv. about 1.25 mol % to about 1.75 mol % PEG2000-DMG; and
b. a nucleic acid molecule having at least 85% sequence identity to SEQ ID NO:125; wherein the lipid formulation encapsulates the nucleic acid molecule and the lipid nanoparticle has a size of about 60 to about 90 nm.

In yet another aspect, the disclosure provides a method for administering any of the compositions described herein to a subject in need thereof, wherein the composition is administered intramuscularly, subcutaneously, intradermally, transdermally, intranasally, orally, sublingually, intravenously, intraperitoneally, topically, by aerosol, or by a pulmonary route. In specific embodiments, n the composition is administered intramuscularly.

In yet another aspect, the disclosure provides a method of administering any of the compositions described herein to a subject in need thereof, wherein the composition is lyophilized and is reconstituted prior to administration.

In yet another aspect, the disclosure provides a method of ameliorating COVID-19, comprising administering any of the compositions described herein to a subject in need thereof.

In some embodiments, the composition is administered one time. In some embodiments, the composition is administered two times.

In yet another aspect, the dislocusre provides a method of administering a booster dose to a vaccinated subject, comprising administering any of the compositions described herein to a subject who was previously vaccinated against coronavirus.

In some embodiments, the composition is administered at a dosage of about 0.01 μg to about 1,000 μg of nucleic acid.

In some embodiments, the composition is administered at a dosage of about 1, 2, 5, 7.5, or 10 μg of nucleic acid.

In yet another aspect, the disclosure provides a method of inducing an immune response in a subject comprising administering to the subject an effective amount of any of the nucleic acid molecules described herein.

In some embodiments, the nucleic acid molecule may be administered intramuscularly, subcutaneously, intradermally, transdermally, intranasally, orally, sublingually, intravenously, intraperitoneally, topically, by aerosol, or by a pulmonary route.

In yet another aspect, the disclosure provides a method of inducing an immune response in a subject comprising administering to the subject an effective amount of any of the compositions described herein.

In some embodiments, the composition may be administered intramuscularly, subcutaneously, intradermally, transdermally, intranasally, orally, sublingually, intravenously, intraperitoneally, topically, by aerosol, or by a pulmonary route.

In some embodiments, the nucleic acid molecules described herein may be used in inducing an immune response to the first antigenic protein or fragment thereof.

In some embodiments, the nucleic acid molecules described herein may be used in the manufacture of a medicament for inducing an immune response to the first antigenic protein or fragment thereof.

### BRIEF DESCRIPTION OF THE DRAWINGS

FIGS. 1A-1D show design and expression of a SARS-CoV-2 vaccine in mRNA and self-replicating RNA (STARR™) platforms. (1A) Schematic diagram of the SARS-CoV-2 self-replicating STARR™ RNA and mRNA vaccine constructs. The STARR™ construct encodes for the four non-structural proteins, ns1-ns4, from Venezuelan equine encephalitis virus (VEEV) and the SARS-CoV-2 full length spike (S) protein. The mRNA construct codes for the SARS-CoV-2 full length spike S protein. (1B) Physical characteristics and RNA trapping efficiency of the LNP in the mRNA and STARR™ (self-replicating RNA corresponding to SEQ ID NO:125; referred to herein as “STARR™ SARS-CoV-2 RNA”) vaccines. (1C) Western blot detection of SARS-CoV-2 S protein following transfection of HEK293 cells with the STARR™ RNA and mRNA constructs. (1D) In vivo comparison of protein expression following intramuscular (IM) administration of LNP containing luciferase-expressing STARR™ RNA or mRNA. Balb/c mice (n=3/group) were injected IM with 0.2 μg, 2.0 μg and 10.0 μg of STARR™ RNA or mRNA in lipid formulation. Luciferase expression was measured by in vivo bioluminescence on days 1, 3 and 7 post-IM administration. S domain 1=S1, S domain 2=S2, transmembrane domain=TM, cytoplasmic domain=CP.

FIGS. 2A-2I show clinical scores, mouse weights and transcriptomic analysis of immune genes following vaccination with STARR™ RNA or mRNA SARS-CoV-2 vaccine candidates. (2A) C57BL/6 mice were immunized with either PBS, mRNA or STARR™ SARS-CoV-2 RNA (doses 0.2 μg, 2 μg or 10 μg), weight and clinical scores assessed every day, bled at day 1 post-immunization, sacrificed at 7 days post-vaccination and lymph nodes harvested. Gene expression of inflammatory genes and immune genes were measured in whole blood (at day 1) and lymph nodes (at day 7), respectively. (2B) Expression of IFN and inflammatory response genes in whole blood presented as heatmap of z scores. (2C) Lymph node weights at 7 days post-vaccination. Principal component analysis (PCA) of immune gene expression following vaccination with mRNA or STARR™ SARS-CoV-2 RNA at doses (2D) 0.2 μg, (2E) 2 μg and (2F) 10 μg. Volcano plots of fold change of STARR™ SARS-CoV-2 RNA versus mRNA (x-axis) and Log 10 P-value of STARR™ SARS-CoV-2 RNA versus mRNA (y-axis) for doses (2G) 0.2 μg, (2H) 2 μg and (2I) 10 μg.

FIGS. 3A-3J show cellular immune responses following vaccination with SARS-CoV-2 STARR™ RNA and mRNA. C57BL/6 mice (n=5 per group) were immunized with 0.2 μg, 2 μg, or 10 μg of STARR™ RNA or mRNA via IM, sacrificed at day 7 post-vaccination and spleens analyzed for cellular T cell responses by flow-cytometry and ELISPOT. (3A-3B) CD8+ and C) CD4+T effector cells were assessed in vaccinated animals using surface staining for T cell markers and flow-cytometry. (3D-3E) IFNγ+CD8+ T cells and (3F) Ratio of IFNγ+/IL4+CD4+ T cells in spleens of immunized mice were assessed following ex vivo stimulation with PMA/ionomycin (IO) and intracellular staining. (3G-3I) SARS-CoV-2 S protein-specific responses to pooled S protein peptides were assessed using IFNγ ELISPOT assays following vaccination with mRNA (3H) or STARR™ RNA (3I). A schematic of S protein domains is shown in (3J).

FIGS. 4A-4G show humoral responses in multiple mouse strains following immunization with mRNA and STARR™ vaccine candidates. (4A) BALB/c and C57BL/6J mice were immunized via IM with 0.2 μg, 2 μg, or 10 μg of STARR™ RNA or mRNA (n=5/group). Blood sampling was conducted at baseline, and days 10, 19, 30, 40, 50 and 60 post-vaccination for BALB/c and days 10, 20 and 30 for C57BL/6J. (4B-4C) IgM and (4D-4E) IgG against the SARS-CoV-2 S protein over time, assessed using insect cell-derived whole S protein in a Luminex immuno-assay (measured as MFI). IgG endpoint titers to mammalian-derived whole S protein, S1, S2 and receptor binding domain (RBD) proteins at day 30 post-vaccination were assessed in (4F) BALB/c and (4G) C57BL/6J.

FIGS. 5A-5D show that STARR™ SARS-CoV-2 RNA elicits Th1 skewed immune responses. SARS-CoV-2 spike-specific IgG subclasses and the ratio of IgG2a/c/IgG1 at 30 days post-vaccination with STARR™ RNA and mRNA in (5A) BALB/c and (5B) C57BL/6J mice. Th2 cytokine and Th1/Th2 skew in CD4 T cells at day 7 post-vaccination in C57BL/6J mice measured by ICS as (5C) percentage of IL4+CD4 T cells and (5D) ratio of IFNγ+/IL4+CD4+ T cells.

FIGS. 6A-6E show that STARR™ SARS-CoV-2 RNA elicits a higher quality humoral response than mRNA platform. (6A) Avidity of SARS-CoV-2 S protein-specific IgG at day 30 post-immunization was measured using 8M urea washes. (6B) Neutralizing antibody (PRNT50 titers) at day 30 post-vaccination against a clinically isolated live SARS-CoV-2 virus measured in both BALB/c and C57BL/6J. Dashed lines depict the serum dilution range (i.e. from 1:20 to 1:320) tested by PRNT. (6C) PRNT50 and (6D) PRNT70 of SARS-CoV-2 neutralization at day 60 post-vaccination and convalescent sera from COVID-19 patients. (6E) Correlation analysis of Spike-specific IgG endpoint titers against SARS-CoV-2 neutralization (PRNT50). PRNT—plaque reduction neutralization test.

FIGS. 7A-7E show clinical scores, body weight and immune responses to STARR™ SARS-CoV-2 RNA and mRNA following boost at day 30 post-prime in C57BL/6J. (7A) Clinical scores and (7B) percentage of initial body weight following boost vaccinations. (7C) Anti-Spike IgG responses following boost by mRNA and STARR™ SARS-CoV-2 RNA. Grey dashed line marks the experimental assay saturation point. IFN γ+CD8+T effector cells responses (fold change over PBS) in animals either primed or prime & boosted with either (7D) mRNA or (7E) STARR™ SARS-CoV-2 RNA vaccine candidates.

FIGS. 8A-8B show whole blood transcriptomic data at 1-day post-prime vaccination showing Nanostring counts per 50 ng RNA of selected (8A) IFN and (8B) inflammatory genes.

FIGS. 9A-9B show correlation analysis of live SARS-CoV-2 neutralization against binding IgG and IgG subclasses in BALB/c and C57BL/6J mouse strains. (9A) Spearman correlation analysis of SARS-CoV-2 neutralization (PRNT50) against total IgG specific to several SARS-CoV-2 antigens, including S, S1, and RBD recombinant proteins. (9B) Spearman correlation analysis of SARS-CoV-2 neutralization (PRNT50) against SARS-CoV-2 S-specific IgG subclasses (IgG1 and IgG2a or IgG2c).

FIG. 10 shows Kaplan-Meier survival curves for unvaccinated mice (PBS) and mice vaccinated with STARR™ SARS-CoV-2 RNA following challenge with a lethal dose of SARS-CoV-2 virus. Upper line—STARR™ SARS-CoV-2 RNA (2 μg, 10 μg); dropping line—PBS.

FIG. 11 shows that STARR™ SARS-CoV-2 RNA vaccination protects against lung and brain SARS-CoV-2 infection. Viral RNA levels in lungs (FIG. 11, left) and in brains (FIG. 11, right) of unvaccinated mice (PBS) and mice vaccinated with the indicated dose of STARR™ SARS-CoV-2 RNA are shown.

FIG. 12 shows viral titers in lungs of unvaccinated mice (PBS) and mice vaccinated with the indicated dose of STARR™ SARS-CoV-2 RNA following challenge with SARS-CoV-2.

FIG. 13 shows an RNA dose-dependent immunogenicity comparison between G614 and D614 SARS CoV-2 glycoprotein expressed from self-replicating RNA.

FIG. 14 shows a schematic illustrating one aspect of STARR™ technology and lipid-mediated delivery.

FIGS. 15A-15C show duration of luciferase reporter gene expression for self-replicating (replicon) RNA (STARR™), such as (15A) STARR™ FLuc, (15B) STARR™ FLuc IRES-E3L, and (15C) STARR™ FLuc IRES E3L (short 3′ UTR) as compared to mRNA.

FIG. 16A-16D show results of Luminex Assay for anti-SARS-Cov-2 Spike Glycoprotein IgG in two pre-clinical studies. BALB/c mice were vaccinated with increasing RNA doses of self-replicating RNA (SEQ ID NO:125) formulated as lyophilized lipid nanoparticles (LYO-LNP) and liquid (frozen) lipid nanoparticles (Liquid-LNP). (16A) First Study 0.2 μg, (16B) First Study 2 μg, (16C) Second Study 0.2 μg, and (16D) Second Study 2 μg. Blood was collected and processed to serum at various times post-vaccination and evaluated for anti-SARS-CoV-2 spike glycoprotein IgG. Two way ANOVA, Tukey's multiple comparison post-test compared LYO-LNP to Liquid-LNP where *p<0.0332, **p<0.0021, ***p<0.0002, ****p<0.0001.

FIGS. 17A-17B show the Area Under the Curve (AUC) Analysis for anti-SARS-Cov-2 Spike Glycoprotein IgG (First and Second Study combined data). IgG assay results were combined from two studies to evaluate self-replicating RNA (SEQ ID NO:125) formulated as lyophilized lipid nanoparticles (LYO-LNP) and liquid (frozen) lipid nanoparticles (Liquid-LNP) at (17A) 0.2 μg, and (17B) 2 μg. N=10/group. First Study Day 19 and 31 results were combined with Second Study Day 20 and 30 results, respectively, and an Area Under the Curve (AUC) analysis was performed. One way ANOVA, Sidak's multiple comparison post-test compared LYO-LNP to Liquid-LNP and resulted in no statistical differences.

FIGS. 18A-18D shows characterization of STARR™ technology with firefly luciferase transgene expression. (18A) Firefly luciferase (FLuc) expression from STARR™ Fluc, SINV FLuc, and mRNA FLuc was monitored up to day 28 by In Vivo Imaging System (IVIS). The average of total flux (p/s) from 6 injection sites in a mouse group was plotted at each time point with a standard error of mean, SEM. (18B) IVIS picture of three mice (6 injection sites) per group on day 14 is shown for each group that was administered with the test article labeled below the picture. (18C) Luciferase expression from mice that were intramuscularly injected with STARR™ FLuc was monitored by IVIS up to 63 days post administration. (18D) Effect of prior administration of replicon backbone was examined for STARR™ (upper panel) and SINV (lower panel). Replicon encoding FLuc was IM injected at 7 days post dose of replicon with homologous backbone with an irrelevant gene/sequence (labeled STARR™ irr or SINV irr) at day 0. As a reference, a mouse group with PBS administration at day 0 was included in each of STARR™ and SINV group.

FIG. 19 shows that STARR™ elicits antigen-specific IFN-gamma response. Enzyme-linked immune absorbent spot ELISpot was used to count the number of splenocytes that were specifically stimulated by an antigen peptide of the same amino acid sequence encoded in TA STARR™. Neither no peptide (cell only) nor irrelevant peptide (Bgal) did not elicit significant IFN-gamma from splenocytes from mice vaccinated with STARR™ FLuc or TA STARR™. Stimulation with AH1-A5 peptide resulted in the detection of IFN-gamma-producing cells specifically from the mice that were vaccinated with TASTARR™. Concanavalin A (ConA) was used as a positive control of IFN-gamma production.

FIGS. 20A-20F illustrate reduced tumor growth rate by TA STARR™ vaccination in a CT26 syngeneic mouse model. CT26 murine colorectal carcinoma cells (5×10⁵) were subcutaneously implanted in 10-week old female BALB/c mice (n=8 per group). On days 1 and 8, the mice were vaccinated with STARR™ FLuc, a negative control, or TA STARR™, which encodes AH1A5 epitope. Tumor growth was monitored in mice vaccinated with (20A) STARR™ FLuc without checkpoint inhibitor treatment; (20B) STARR™ FLuc with a combination anti-PD1/PDL1 treatment; (20C) STARR™ FLuc with a combination anti-CTLA4 treatment; (20D) STARR™ vaccine without checkpoint inhibitor treatment; (20E) STARR™ vaccine with a combination treatment of anti-PD1 and anti-PDL1; and (20F) STARR™ vaccine with a combination treatment of anti-CTLA4. The individual tumor growth curves from a mouse group that were administered with STARR™ FLuc and TA STARR™ are shown in upper and lower panels, respectively.

FIG. 21 illustrates prolonged protection by combination treatment of TA STARR™ Vaccine with checkpoint inhibitors. Mice that were treated with TA STARR™ combined with anti-PD1/PDL1 or anti-CTLA4 were found to be resistant to tumor growth following the CT26 challenge at day 25 to 42. Naïve mice were used as a control for the CT26 tumor growth.

FIGS. 22A-22C show results from AH1-tetramer staining of CD8+ T-cells in the form of (22A) a graph and (22B and 22C) plots. Splenocytes from the mice group with combination treatment of TA STARR™ and anti-PD1/PDL1 at day 42 were stained with AH1 (H-2Ld)-tetramer. The staining was specific to CD8+ T cells from the mouse group with TA STARR™ treatment, and the population represented 9-17% of total CD8+ T cells from the splenocytes.

FIG. 23 shows HAI titers obtained for self-replicating RNA (STARR™) and mRNA constructs encoding the hemagglutinin of influenza virus A/California/07/2009 (H1N1).

FIGS. 24A-24B show RNA replication levels (FIG. 24A) and luciferase reporter gene expression levels (FIG. 24B) for the indicated self-replicating (replicon) RNAs as compared to mRNA.

### DETAILED DESCRIPTION

The present disclosure relates to self-replicating RNAs and nucleic acids encoding the same for expression of transgenes such as antigenic proteins and tumor antigens, for example. Also provided herein are methods of administration (e.g., to a host, such as a mammalian subject) of self-replicating RNAs, whereby the self-replicating RNA is translated in vivo and the heterologous protein-coding sequence is expressed and, e.g., can elicit an immune response to the heterologous protein-coding sequence in the recipient or provide a therapeutic effect, where the heterologous protein-coding sequence is a therapeutic protein. Self-replicating RNAs provided herein are useful as vaccines that can be rapidly generated and that can be effective at low and/or single doses. The present disclosure further relates to methods of inducing an immune response using self-replicating RNAs provided herein.

In some embodiments, an immune response can be elicited against Coronavirus: immunogens that include, but are not limited to, those derived from a SARS coronavirus, avian infectious bronchitis (IBV), Mouse hepatitis virus (MHV), and Porcine transmissible gastroenteritis virus (TGEV). The coronavirus immunogen may be a spike polypeptide.

Self-replicating RNAs are described, for example, in U.S. 2018/0036398, the contents of which are incorporated by reference in their entirety.

### Definitions

As used herein, the term “fragment,” when referring to a protein or nucleic acid, for example, means any shorter sequence than the full-length protein or nucleic acid. Accordingly, any sequence of a nucleic acid or protein other than the full-length nucleic acid or protein sequence can be a fragment. In some aspects, a protein fragment includes an epitope. In other aspects, a protein fragment is an epitope.

As used herein, the term “nucleic acid” refers to any deoxyribonucleic acid (DNA) molecule, ribonucleic acid (RNA) molecule, or nucleic acid analogues. A DNA or RNA molecule can be double-stranded or single-stranded and can be of any size. Exemplary nucleic acids include, but are not limited to, chromosomal DNA, plasmid DNA, cDNA, cell-free DNA (cfDNA), mitochondrial DNA, chloroplast DNA, viral DNA, mRNA, tRNA, rRNA, long non-coding RNA, siRNA, micro RNA (miRNA or miR), hnRNA, and viral RNA. Exemplary nucleic analogues include peptide nucleic acid, morpholino- and locked nucleic acid, glycol nucleic acid, and threose nucleic acid. As used herein, the term “nucleic acid molecule” is meant to include fragments of nucleic acid molecules as well as any full-length or non-fragmented nucleic acid molecule, for example. As used herein, the terms “nucleic acid” and “nucleic acid molecule” can be used interchangeably, unless context clearly indicates otherwise.

As used herein, the term “protein” refers to any polymeric chain of amino acids. The terms “peptide” and “polypeptide” can be used interchangeably with the term protein, unless context clearly indicates otherwise, and can also refer to a polymeric chain of amino acids. The term “protein” encompasses native or artificial proteins, protein fragments and polypeptide analogs of a protein sequence. A protein may be monomeric or polymeric. The term “protein” encompasses fragments and variants (including fragments of variants) thereof, unless otherwise contradicted by context.

In general, “sequence identity” or “sequence homology,” which can be used interchangeably, refer to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby or the amino acid sequence of a polypeptide, and comparing these sequences to a second nucleotide or amino acid sequence. As used herein, the term “percent (%) sequence identity” or “percent (%) identity,” also including “homology,” refers to the percentage of amino acid residues or nucleotides in a sequence that are identical with the amino acid residues or nucleotides in a reference sequence after aligning the sequences and introducing gaps, if necessary, to achieve the maximum percent sequence identity, and not considering any conservative substitutions as part of the sequence identity. Thus, two or more sequences (polynucleotide or amino acid) can be compared by determining their “percent identity,” also referred to as “percent homology.” The percent identity to a reference sequence (e.g., nucleic acid or amino acid sequences), which may be a sequence within a longer molecule (e.g., polynucleotide or polypeptide), may be calculated as the number of exact matches between two optimally aligned sequences divided by the length of the reference sequence and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul et al., J. Mol. Biol. 215:403-410 (1990); Karlin and Altschul, Proc. Natl. Acad. sci. USA 90:5873-5877 (1993); and Altschul et al., Nucleic Acids Res. 25:3389-3402 (1997). Briefly, the BLAST program defines identity as the number of identical aligned symbols (i.e., nucleotides or amino acids), divided by the total number of symbols in the shorter of the two sequences. The program may be used to determine percent identity over the entire length of the sequences being compared. Default parameters are provided to optimize searches with short query sequences, for example, with the blastp program. The program also allows use of an SEG filter to mask-off segments of the query sequences as determined by the SEG program of Wootton and Federhen, Computers and Chemistry 17: 149-163 (1993). Ranges of desired degrees of sequence identity are approximately 80% to 100% and integer values in between. Percent identities between a reference sequence and a claimed sequence can be at least 80%, at least 85%, at least 90%, at least 95%, at least 98%, at least 99%, at least 99.5%, or at least 99.9%. In general, an exact match indicates 100% identity over the length of the reference sequence. Additional programs and methods for comparing sequences and/or assessing sequence identity include the Needleman-Wunsch algorithm (see, e.g., the EMBOSS Needle aligner available at ebi.ac.uk/Tools/psa/emboss needle/, optionally with default settings), the Smith-Waterman algorithm (see, e.g., the EMBOSS Water aligner available at ebi.ac.uk/Tools/psa/emboss water/, optionally with default settings), the similarity search method of Pearson and Lipman, 1988, Proc. Natl. Acad. Sci. USA 85, 2444, or computer programs which use these algorithms (GAP, BESTFIT, FASTA, BLAST P, BLAST N and TFASTA in Wisconsin Genetics Software Package, Genetics Computer Group. 575 Science Drive, Madison, Wis.). In some aspects, reference to percent sequence identity refers to sequence identity as measured using BLAST (Basic Local Alignment Search Tool). In other aspects, ClustalW is used for multiple sequence alignment. Optimal alignment may be assessed using any suitable parameters of a chosen algorithm, including default parameters.

As used herein, the term “drug” or “medicament,” means a pharmaceutical formulation or composition as described herein.

As used herein, the singular forms “a,” “an,” and “the” include plural references unless the context clearly dictates otherwise. Thus, for example, references to “the method” includes one or more methods, and/or steps of the type described herein which will become apparent to those persons skilled in the art upon reading this disclosure and so forth.

“About” as used herein when referring to a measurable value such as an amount, a temporal duration, and the like, is meant to encompass variations of +20%, or ±10%, or ±5%, or even ±1% from the specified value, as such variations are appropriate for the disclosed methods or to perform the disclosed methods.

The term “expression” refers to the process by which a nucleic acid sequence or a polynucleotide is transcribed from a DNA template (such as into mRNA or other RNA transcript) and/or the process by which a transcribed mRNA or other RNA is subsequently translated into peptides, polypeptides, or proteins. Transcripts and encoded polypeptides may be collectively referred to as “gene product.”

As used herein, the terms “self-replicating RNA,” “self-transcribing and self-replicating RNA,” “self-amplifying RNA (saRNA),” and “replicon” may be used interchangeably, unless context clearly indicates otherwise. Generally, the term “replicon” or “viral replicon” refers to a self-replicating subgenomic RNA derived from a viral genome that includes viral genes encoding non-structural proteins important for viral replication and that lacks viral genes encoding structural proteins. A self-replicating RNA can encode further subgenomic RNAs that are not able to self-replicate.

As used herein, “operably linked,” “operable linkage,” “operatively linked,” or grammatical equivalents thereof refer to juxtaposition of genetic elements, e.g., a promoter, an enhancer, a polyadenylation sequence, etc., wherein the elements are in a relationship permitting them to operate in the expected manner. For instance, a regulatory element, which can comprise promoter and/or enhancer sequences, is operatively linked to a coding region if the regulatory element helps initiate transcription of the coding sequence. There may be intervening residues between the regulatory element and coding region so long as this functional relationship is maintained.

### Nucleic Acid Molecules

In some embodiments, provided herein are nucleic acid molecules comprising: (i) a first polynucleotide encoding one or more viral replication proteins, wherein the first polynucleotide is codon-optimized as compared to a wild-type polynucleotide encoding the one or more viral replication proteins; and (ii) a second polynucleotide comprising a first transgene encoding a first antigenic protein or a fragment thereof, wherein the first antigenic protein is a coronavirus protein.

An RNA molecule can encode a single polypeptide immunogen or multiple polypeptides. Multiple immunogens can be presented as a single polypeptide immunogen (fusion polypeptide) or as separate polypeptides. If immunogens are expressed as separate polypeptides from a replicon then one or more of these may be provided with an upstream IRES or an additional viral promoter element. Alternatively, multiple immunogens may be expressed from a polyprotein that encodes individual immunogens fused to a short autocatalytic protease (e.g. foot-and-mouth disease virus 2A protein), or as inteins.

Also provided herein, in some embodiments, are nucleic acid molecules comprising: (i) a first polynucleotide comprising a sequence having at least 80% identity to a sequence of SEQ ID NO:72; and (ii) a second polynucleotide comprising a first transgene encoding a first antigenic protein or a fragment thereof.

### Codon Optimization

In some embodiments, first polynucleotides of nucleic acid molecules provided herein encoding one or more viral replication proteins include codon-optimized sequences. As used herein, the term “codon-optimized” means a polynucleotide, nucleic acid sequence, or coding sequence has been redesigned as compared to a wild-type or reference polynucleotide, nucleic acid sequence, or coding sequence by choosing different codons without altering the amino acid sequence of the encoded protein. Accordingly, codon-optimization generally refers to replacement of codons with synonymous codons to optimize expression of a protein while keeping the amino acid sequence of the translated protein the same. Codon optimization of a sequence can increase protein expression levels (Gustafsson et al., Codon bias and heterologous protein expression. 2004, Trends Biotechnol 22: 346-53) of the encoded proteins, for example, and provide other advantages. Variables such as codon usage preference as measured by codon adaptation index (CAI), for example, the presence or frequency of U and other nucleotides, mRNA secondary structures, cis-regulatory sequences, GC content, and other variables may correlate with protein expression levels (Villalobos et al., Gene Designer: a synthetic biology tool for constructing artificial DNA segments. 2006, BMC Bioinformatics 7:285).

Any method of codon optimization can be used to codon optimize polynucleotides and nucleic acid molecules provided herein, and any variable can be altered by codon optimization. Accordingly, any combination of codon optimization methods can be used. Exemplary methods include the high codon adaptation index (CAI) method, the Low U method, and others. The CAI method chooses a most frequently used synonymous codon for an entire protein coding sequence. As an example, the most frequently used codon for each amino acid can be deduced from 74,218 protein-coding genes from a human genome. The Low U method targets U-containing codons that can be replaced with a synonymous codon with fewer U moieties, generally without changing other codons. If there is more than one choice for replacement, the more frequently used codon can be selected. Any polynucleotide, nucleic acid sequence, or codon sequence provided herein can be codon-optimized.

In some embodiments, the nucleotide sequence of any region of the RNA or DNA templates described herein may be codon optimized. Preferably, the primary cDNA template may include reducing the occurrence or frequency of appearance of certain nucleotides in the template strand. For example, the occurrence of a nucleotide in a template may be reduced to a level below 25% of said nucleotides in the template. In further examples, the occurrence of a nucleotide in a template may be reduced to a level below 20% of said nucleotides in the template. In some examples, the occurrence of a nucleotide in a template may be reduced to a level below 16% of said nucleotides in the template. Preferably, the occurrence of a nucleotide in a template may be reduced to a level below 15%, and preferably may be reduced to a level below 12% of said nucleotides in the template.

In some embodiments, the nucleotide reduced is uridine. For example, the present disclosure provides nucleic acids with altered uracil content wherein at least one codon in the wild-type sequence has been replaced with an alternative codon to generate a uracil-altered sequence. Altered uracil sequences can have at least one of the following properties:
(i) an increase or decrease in global uracil content (i.e., the percentage of uracil of the total nucleotide content in the nucleic acid of a section of the nucleic acid, e.g., the open reading frame);
(ii) an increase or decrease in local uracil content (i.e., changes in uracil content are limited to specific subsequences);
(iii) a change in uracil distribution without a change in the global uracil content;
(iv) a change in uracil clustering (e.g., number of clusters, location of clusters, or distance between clusters); or
(v) combinations thereof.

In some embodiments, the percentage of uracil nucleobases in the nucleic acid sequence is reduced with respect to the percentage of uracil nucleobases in the wild-type nucleic acid sequence. For example, 30% of nucleobases may be uracil in the wild-type sequence but the nucleobases that are uracil are preferably lower than 15%, preferably lower than 12% and preferably lower than 10% of the nucleobases in the nucleic acid sequences of the disclosure. The percentage uracil content can be determined by dividing the number of uracil in a sequence by the total number of nucleotides and multiplying by 100.

In some embodiments, the percentage of uracil nucleobases in a subsequence of the nucleic acid sequence is reduced with respect to the percentage of uracil nucleobases in the corresponding subsequence of the wild-type sequence. For example, the wild-type sequence may have a 5′-end region (e.g., 30 codons) with a local uracil content of 30%, and the uracil content in that same region could be reduced to preferably 15% or lower, preferably 12% or lower and preferably 10% or lower in the nucleic acid sequences of the disclosure. These subsequences can also be part of the wild-type sequences of the heterologous 5′ and 3′ UTR sequences of the present disclosure.

In some embodiments, codons in the nucleic acid sequence of the disclosure reduce or modify, for example, the number, size, location, or distribution of uracil clusters that could have deleterious effects on protein translation. Although lower uracil content is desirable in certain aspects, the uracil content, and in particular the local uracil content, of some subsequences of the wild-type sequence can be greater than the wild-type sequence and still maintain beneficial features (e.g., increased expression).

In some embodiments, the uracil-modified sequence induces a lower Toll-Like Receptor (TLR) response when compared to the wild-type sequence. Several TLRs recognize and respond to nucleic acids. Double-stranded (ds)RNA, a frequent viral constituent, has been shown to activate TLR3. Single-stranded (ss)RNA activates TLR7. RNA oligonucleotides, for example RNA with phosphorothioate internucleotide linkages, are ligands of human TLR8. DNA containing unmethylated CpG motifs, characteristic of bacterial and viral DNA, activate TLR9.

As used herein, the term “TLR response” is defined as the recognition of single-stranded RNA by a TLR7 receptor, and preferably encompasses the degradation of the RNA and/or physiological responses caused by the recognition of the single-stranded RNA by the receptor. Methods to determine and quantify the binding of an RNA to a TLR7 are known in the art. Similarly, methods to determine whether an RNA has triggered a TLR7-mediated physiological response (e.g., cytokine secretion) are well known in the art. In some embodiments, a TLR response can be mediated by TLR3, TLR8, or TLR9 instead of TLR7. Suppression of TLR7-mediated response can be accomplished via nucleoside modification. RNA undergoes over a hundred different nucleoside modifications in nature. Human rRNA, for example, has ten times more pseudouracil (′P) and 25 times more 2′-O-methylated nucleosides than bacterial rRNA. Bacterial RNA contains no nucleoside modifications, whereas mammalian RNAs have modified nucleosides such as 5-methylcytidine (m5C), N6-methyladenosine (m6A), inosine and many 2′-O-methylated nucleosides in addition to N7-methylguanosine (m7G).

In some embodiments, the uracil content of polynucleotides disclosed herein is less than about 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2% or 1% of the total nucleobases in the sequence in the reference sequence. In some embodiments, the uracil content of polynucleotides disclosed herein is between about 5% and about 25%. In some embodiments, the uracil content of polynucleotides disclosed herein is between about 15% and about 25%.

In some embodiments, first polynucleotides of nucleic acid molecules provided herein comprise a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, and any number or range in between, identity to a sequence of SEQ ID NO:72. In some embodiments, first polynucleotides of nucleic acid molecules provided herein comprise a sequence of SEQ ID NO:72.

In some aspects, first polynucleotides and second polynucleotides of nucleic acid molecules provided herein are included in the same (i.e., a single) or in separate nucleic acid molecules. Generally, first polynucleotides and second polynucleotides of nucleic acid molecules provided herein are included in a single nucleic acid molecule. In one aspect, the first polynucleotide is located 5′ of the second polynucleotide. In one aspect, first polynucleotides and second polynucleotides of nucleic acid molecules provided herein are included in separate nucleic acid molecules. In yet another aspect, first polynucleotides and second polynucleotides are included in two separate nucleic acid molecules.

In some aspects, first polynucleotides and second polynucleotides are included in the same (i.e., a single) nucleic acid molecule. First polynucleotides and second polynucleotides of nucleic acid molecules provided herein can be contiguous, i.e., adjacent to each other without nucleotides in between. In one aspect, an intergenic region is located between the first polynucleotide and the second polynucleotide. In another aspect, the intergenic region located between the first polynucleotide and the second polynucleotide is a second intergenic region, with a first intergenic region included in the first polynucleotide as described below. As used herein, the terms “intergenic region” and intergenic sequence” can be used interchangeably, unless context clearly indicates otherwise.

An intergenic region located between the first polynucleotide and the second polynucleotide can be of any length and can have any nucleotide sequence. As an example, the intergenic region between the first polynucleotide and the second polynucleotide can include about one nucleotide, about two nucleotides, about three nucleotides, about four nucleotides, about five nucleotides, about six nucleotides, about seven nucleotides, about eight nucleotides, about nine nucleotides, about ten nucleotides, about 11 nucleotides, about 12 nucleotides, about 13 nucleotides, about 14 nucleotides, about 15 nucleotides, about 16 nucleotides, about 17 nucleotides, about 18 nucleotides, about 19 nucleotides, about 20 nucleotides, about 21 nucleotides, about 22 nucleotides, about 23 nucleotides, about 24 nucleotides, about 25 nucleotides, about 26 nucleotides, about 27 nucleotides, about 28 nucleotides, about 29 nucleotides, about 30 nucleotides, about 31 nucleotides, about 32 nucleotides, about 33 nucleotides, about 34 nucleotides, about 35 nucleotides, about 36 nucleotides, about 37 nucleotides, about 38 nucleotides, about 39 nucleotides, about 40 nucleotides, about 41 nucleotides, about 42 nucleotides, about 43 nucleotides, about 44 nucleotides, about 45 nucleotides, about 46 nucleotides, about 47 nucleotides, about 48 nucleotides, about 49 nucleotides, about 50 nucleotides, about 60 nucleotides, about 70 nucleotides, about 80 nucleotides, about 90 nucleotides, about 100 nucleotides, about 125 nucleotides, about 150 nucleotides, about 175 nucleotides, about 200 nucleotides, about 250 nucleotides, about 300 nucleotides, about 350 nucleotides, about 400 nucleotides, about 450 nucleotides, about 500 nucleotides, about 600 nucleotides, about 700 nucleotides, about 800 nucleotides, about 1,000 nucleotides, about 1,500 nucleotides, about 2,000 nucleotides, about 2,500 nucleotides, about 3,000 nucleotides, about 3,500 nucleotides, about 4,000 nucleotides, about 4,500 nucleotides, about 5,000 nucleotides, about 6,000 nucleotides, about 7,000 nucleotides, about 8,000 nucleotides, about 9,000 nucleotides, about 10,000 nucleotides, and any number or range in between. In one aspect, the intergenic region between first and second polynucleotides includes about 10-100 nucleotides, about 10-200 nucleotides, about 10-300 nucleotides, about 10-400 nucleotides, or about 10-500 nucleotides. In another aspect, the intergenic region between first and second polynucleotides includes about 1-10 nucleotides, about 1-20 nucleotides, about 1-30 nucleotides, about 1-40 nucleotides, or about 1-50 nucleotides. In yet another aspect, the region includes about 44 nucleotides. In one aspect, the intergenic region between first and second polynucleotides of nucleic acid molecules provided herein is a second intergenic region.

In one aspect, the intergenic region between first and second polynucleotides includes a viral sequence. The intergenic region between first and second polynucleotides can include a sequence from any virus, such as alphaviruses and rubiviruses, for example. In one aspect, the intergenic region between the first polynucleotide and the second polynucleotide comprises an alphavirus sequence, such as a sequence from Venezuelan Equine Encephalitis Virus (VEEV), Eastern Equine Encephalitis Virus (EEEV), Everglades Virus (EVEV), Mucambo Virus (MUCV), Semliki Forest Virus (SFV), Pixuna Virus (PIXV), Middleburg Virus (MIDV), Chikungunya Virus (CHIKV), O'Nyong-Nyong Virus (ONNV), Ross River Virus (RRV), Barmah Forest Virus (BFV), Getah Virus (GETV), Sagiyama Virus (SAGV), Bebaru Virus (BEBV), Mayaro Virus (MAYV), Una Virus (UNAV), Sindbis Virus (SINV), Aura Virus (AURAV), Whataroa Virus (WHAV), Babanki Virus (BABV), Kyzylagach Virus (KYZV), Western Equine Encephalitis Virus (WEEV), Highland J Virus (HJV), Fort Morgan Virus (FMV), Ndumu Virus (NDUV), Salmonid Alphavirus (SAV), Buggy Creek Virus (BCRV), or any combination thereof. In another aspect, the intergenic region between first and second polynucleotides comprises a sequence from Venezuelan Equine Encephalitis Virus (VEEV). In yet another aspect, the intergenic region between first and second polynucleotides comprises a sequence having at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, and any number or range in between, identity to SEQ ID NO:77. In a further aspect, the intergenic region between first and second polynucleotides comprises a sequence of SEQ ID NO:77. In yet a further aspect, the intergenic region between first and second polynucleotides is a second intergenic region comprising a sequence having at least 85% identity to SEQ ID NO:77.

### Natural and Modified Nucleotides

A self-replicating RNA of the disclosure can comprise one or more chemically modified nucleotides. Examples of nucleic acid monomers include non-natural, modified, and chemically-modified nucleotides, including any such nucleotides known in the art. Nucleotides can be artificially modified at either the base portion or the sugar portion. In nature, most polynucleotides comprise nucleotides that are “unmodified” or “natural” nucleotides, which include the purine bases adenine (A) and guanine (G), and the pyrimidine bases thymine (T), cytosine (C) and uracil (U). These bases are typically fixed to a ribose or deoxy ribose at the 1′ position. The use of RNA polynucleotides comprising chemically modified nucleotides have been shown to improve RNA expression, expression rates, half-life and/or expressed protein concentrations. RNA polynucleotides comprising chemically modified nucleotides have also been useful in optimizing protein localization thereby avoiding deleterious bio-responses such as immune responses and/or degradation pathways.

Examples of modified or chemically-modified nucleotides include 5-hydroxycytidines, 5-alkylcytidines, 5-hydroxyalkylcytidines, 5-carboxycytidines, 5-formylcytidines, 5-alkoxycytidines, 5-alkynylcytidines, 5-halocytidines, 2-thiocytidines, N4-alkylcytidines, N4-aminocytidines, N4-acetylcytidines, and N4,N4-dialkylcytidines.

Examples of modified or chemically-modified nucleotides include 5-hydroxycytidine, 5-methylcytidine, 5-hydroxymethylcytidine, 5-carboxycytidine, 5-formylcytidine, 5-methoxycytidine, 5-propynylcytidine, 5-bromocytidine, 5-iodocytidine, 2-thiocytidine; N4-methylcytidine, N4-aminocytidine, N4-acetylcytidine, and N4,N4-dimethylcytidine.

Examples of modified or chemically-modified nucleotides include 5-hydroxyuridines, 5-alkyluridines, 5-hydroxyalkyluridines, 5-carboxyuridines, 5-carboxyalkylesteruridines, 5-formyluridines, 5-alkoxyuridines, 5-alkynyluridines, 5-halouridines, 2-thiouridines, and 6-alkyluridines.

Examples of modified or chemically-modified nucleotides include 5-hydroxyuridine, 5-methyluridine, 5-hydroxymethyluridine, 5-carboxyuridine, 5-carboxymethylesteruridine, 5-formyluridine, 5-methoxyuridine (also referred to herein as “5MeOU”), 5-propynyluridine, 5-bromouridine, 5-fluorouridine, 5-iodouridine, 2-thiouridine, and 6-methyluridine.

Examples of modified or chemically-modified nucleotides include 5-methoxycarbonylmethyl-2-thiouridine, 5-methylaminomethyl-2-thiouridine, 5-carbamoylmethyluridine, 5-carbamoylmethyl-2′-O-methyluridine, 1-methyl-3-(3-amino-3-carboxypropy)pseudouridine, 5-methylaminomethyl-2-selenouridine, 5-carboxymethyluridine, 5-methyldihydrouridine, 5-taurinomethyluridine, 5-taurinomethyl-2-thiouridine, 5-(isopentenylaminomethyl)uridine, 2′-O-methylpseudouridine, 2-thio-2′O-methyluridine, and 3,2′-O-dimethyluridine.

Examples of modified or chemically-modified nucleotides include N6-methyladenosine, 2-aminoadenosine, 3-methyladenosine, 8-azaadenosine, 7-deazaadenosine, 8-oxoadenosine, 8-bromoadenosine, 2-methylthio-N6-methyladenosine, N6-isopentenyladenosine, 2-methylthio-N6-isopentenyladenosine, N6-(cis-hydroxyisopentenyl)adenosine, 2-methylthio-N6-(cis-hydroxyisopentenyl)adenosine, N6-glycinylcarbamoyladenosine, N6-threonylcarbamoyl-adenosine, N6-methyl-N6-threonylcarbamoyl-adenosine, 2-methylthio-N6-threonylcarbamoyl-adenosine, N6,N6-dimethyladenosine, N6-hydroxynorvalylcarbamoyladenosine, 2-methylthio-N6-hydroxynorvalylcarbamoyl-adenosine, N6-acetyl-adenosine, 7-methyl-adenine, 2-methylthio-adenine, 2-methoxy-adenine, alpha-thio-adenosine, 2′-O-methyl-adenosine, N6,2′-O-dimethyl-adenosine, N6,N6,2′-O-trimethyl-adenosine, 1,2′-O-dimethyl-adenosine, 2′-O-ribosyladenosine, 2-amino-N6-methyl-purine, 1-thio-adenosine, 2′-F-ara-adenosine, 2′-F-adenosine, 2′-OH-ara-adenosine, and N6-(19-amino-pentaoxanonadecyl)-adenosine.

Examples of modified or chemically-modified nucleotides include N1-alkylguanosines, N2-alkylguanosines, thienoguanosines, 7-deazaguanosines, 8-oxoguanosines, 8-bromoguanosines, O6-alkylguanosines, xanthosines, inosines, and N1-alkylinosines.

Examples of modified or chemically-modified nucleotides include N1-methylguanosine, N2-methylguanosine, thienoguanosine, 7-deazaguanosine, 8-oxoguanosine, 8-bromoguanosine, O6-methylguanosine, xanthosine, inosine, and N1-methylinosine.

Examples of modified or chemically-modified nucleotides include pseudouridines. Examples of pseudouridines include N1-alkylpseudouridines, N1-cycloalkylpseudouridines, N1-hydroxypseudouridines, N1-hydroxyalkylpseudouridines, N1-phenylpseudouridines, N1-phenylalkylpseudouridines, N1-aminoalkylpseudouridines, N3-alkylpseudouridines, N6-alkylpseudouridines, N6-alkoxypseudouridines, N6-hydroxypseudouridines, N6-hydroxyalkylpseudouridines, N6-morpholinopseudouridines, N6-phenylpseudouridines, and N6-halopseudouridines. Examples of pseudouridines include N1-alkyl-N6-alkylpseudouridines, N1-alkyl-N6-alkoxypseudouridines, N1-alkyl-N6-hydroxypseudouridines, N1-alkyl-N6-hydroxyalkylpseudouridines, N1-alkyl-N6-morpholinopseudouridines, N1-alkyl-N6-phenylpseudouridines, and N1-alkyl-N6-halopseudouridines. In these examples, the alkyl, cycloalkyl, and phenyl substituents may be unsubstituted, or further substituted with alkyl, halo, haloalkyl, amino, or nitro substituents.

Examples of pseudouridines include N1-methylpseudouridine (also referred to herein as “NIMPU”), N1-ethylpseudouridine, N1-propylpseudouridine, N1-cyclopropylpseudouridine, N1-phenylpseudouridine, N1-aminomethylpseudouridine, N3-methylpseudouridine, N1-hydroxypseudouridine, and N1-hydroxymethylpseudouridine.

Examples of nucleic acid monomers include modified and chemically-modified nucleotides, including any such nucleotides known in the art.

Examples of modified and chemically-modified nucleotide monomers include any such nucleotides known in the art, for example, 2′-O-methyl ribonucleotides, 2′-O-methyl purine nucleotides, 2′-deoxy-2′-fluoro ribonucleotides, 2′-deoxy-2′-fluoro pyrimidine nucleotides, 2′-deoxy ribonucleotides, 2′-deoxy purine nucleotides, universal base nucleotides, 5-C-methyl-nucleotides, and inverted deoxyabasic monomer residues.

Examples of modified and chemically-modified nucleotide monomers include 3′-end stabilized nucleotides, 3′-glyceryl nucleotides, 3′-inverted abasic nucleotides, and 3′-inverted thymidine.

Examples of modified and chemically-modified nucleotide monomers include locked nucleic acid nucleotides (LNA), 2′-O,4′-C-methylene-(D-ribofuranosyl) nucleotides, 2′-methoxyethoxy (MOE) nucleotides, 2′-methyl-thio-ethyl, 2′-deoxy-2′-fluoro nucleotides, and 2′-O-methyl nucleotides. In an exemplary embodiment, the modified monomer is a locked nucleic acid nucleotide (LNA).

Examples of modified and chemically-modified nucleotide monomers include 2′,4′-constrained 2′-O-methoxyethyl (cMOE) and 2′-O-Ethyl (cEt) modified DNAs.

Examples of modified and chemically-modified nucleotide monomers include 2′-amino nucleotides, 2′-O-amino nucleotides, 2′-C-allyl nucleotides, and 2′-O-allyl nucleotides.

Examples of modified and chemically-modified nucleotide monomers include N6-methyladenosine nucleotides.

Examples of modified and chemically-modified nucleotide monomers include nucleotide monomers with modified bases 5-(3-amino)propyluridine, 5-(2-mercapto)ethyluridine, 5-bromouridine; 8-bromoguanosine, or 7-deazaadenosine.

Examples of modified and chemically-modified nucleotide monomers include 2′-O-aminopropyl substituted nucleotides.

Examples of modified and chemically-modified nucleotide monomers include replacing the 2′—OH group of a nucleotide with a 2′-R, a 2′-OR, a 2′-halogen, a 2′-SR, or a 2′-amino, where R can be H, alkyl, alkenyl, or alkynyl.

Example of base modifications described above can be combined with additional modifications of nucleoside or nucleotide structure, including sugar modifications and linkage modifications. Certain modified or chemically-modified nucleotide monomers may be found in nature.

Preferred nucleotide modifications include N1-methylpseudouridine and 5-methoxyuridine.

### Viral Replication Proteins and Polynucleotides Encoding Them

Provided herein, in some embodiments, are nucleic acid molecules comprising a first polynucleotide encoding one or more viral replication proteins. As used herein, the term “replication protein” or “viral replication protein” refers to any protein or any protein subunit of a protein complex that functions in replication of a viral genome. Generally, viral replication proteins are non-structural proteins. Viral replication proteins encoded by nucleic acid molecules provided herein can function in the replication of any viral genome. The viral genome can be a single-stranded positive-sense RNA genome, a single-stranded negative-sense RNA genome, a double-stranded RNA genome, a single-stranded positive-sense DNA genome, a single-stranded negative-sense DNA genome, or a double-stranded DNA genome. Viral genomes can include a single nucleic acid molecule or more than one nucleic acid molecule. Nucleic acid molecules provided herein can encode one or more viral replication proteins from any virus or virus family, including animal viruses and plant viruses, for example. Viral replication proteins encoded by first polynucleotides included in nucleic acid molecules provided herein can be expressed from self-replicating RNA.

First polynucleotide sequences of nucleic acid molecules provided herein can encode one or more togavirus replication proteins. In some aspects, the one or more viral replication proteins encoded by first polynucleotides of nucleic acid molecules provided herein are alphavirus proteins. In some embodiments, the one or more viral replication proteins encoded by first polynucleotides of nucleic acid molecules provided herein are rubivirus proteins. First polynucleotide sequences of nucleic acid molecules provided herein can encode any alphavirus replication protein and any rubivirus replication protein. Exemplary replication proteins from alphaviruses include proteins from Venezuelan Equine Encephalitis Virus (VEEV), Eastern Equine Encephalitis Virus (EEEV), Everglades Virus (EVEV), Mucambo Virus (MUCV), Semliki Forest Virus (SFV), Pixuna Virus (PIXV), Middleburg Virus (MIDV), Chikungunya Virus (CHIKV), O'Nyong-Nyong Virus (ONNV), Ross River Virus (RRV), Barmah Forest Virus (BFV), Getah Virus (GETV), Sagiyama Virus (SAGV), Bebaru Virus (BEBV), Mayaro Virus (MAYV), Una Virus (UNAV), Sindbis Virus (SINV), Aura Virus (AURAV), Whataroa Virus (WHAV), Babanki Virus (BABV), Kyzylagach Virus (KYZV), Western Equine Encephalitis Virus (WEEV), Highland J Virus (HJV), Fort Morgan Virus (FMV), Ndumu Virus (NDUV), Salmonid Alphavirus (SAV), Buggy Creek Virus (BCRV), and any combination thereof. Exemplary rubivirus replication proteins include proteins from rubella virus.

Viral replication proteins encoded by first polynucleotides of nucleic acid molecules provided herein can be expressed as one or more polyproteins or as separate or single proteins. Generally, polyproteins are precursor proteins that are cleaved to generate individual or separate proteins. Accordingly, proteins derived from a precursor polyprotein can be expressed from a single open reading frame (ORF). As used herein, the term “ORF” refers to a nucleotide sequence that begins with a start codon, generally ATG, and that ends with a stop codon, such as TAA, TAG, or TGA, for example. It will be appreciated that T is present in DNA, while U is present in RNA. Accordingly, a start codon of ATG in DNA corresponds to AUG in RNA, and the stop codons TAA, TAG, and TGA in DNA correspond to UAA, UAG, and UGA in RNA. It will further be appreciated that for any sequence provided in the present disclosure, T is present in DNA, while U is present in RNA. Accordingly, for any sequence provided herein, T present in DNA is substituted with U for an RNA molecule, and U present in RNA is substituted with T for a DNA molecule.

The protease cleaving a polyprotein can be a viral protease or a cellular protease. In some aspects, the first polynucleotide of nucleic acid molecules provided herein encodes a polyprotein comprising an alphavirus nsP1 protein, an alphavirus nsP2 protein, an alphavirus nsP3 protein, an alphavirus nsP4 protein, or any combination thereof. In other aspects, the first polynucleotide of nucleic acid molecules provided herein encodes a polyprotein comprising an alphavirus nsP1 protein, an alphavirus nsP2 protein, an alphavirus nsP3 protein, or any combination thereof, and an alphavirus nsP4 protein. In some aspects, the polyprotein is a VEEV polyprotein. In other aspects, the alphavirus nsP1, nsP2, nsP3, and nsP4 proteins are VEEV proteins.

In one aspect, first polynucleotides of nucleic acid molecules provided herein lack a stop codon between sequences encoding an nsP3 protein and an nsP4 protein. Accordingly, in some aspects, first polynucleotides of nucleic acid molecules provided herein encode a P1234 polyprotein comprising nsP1, nsP2, nsP3, and nsP4. First polynucleotides of nucleic acid molecules provided herein can also include a stop codon between sequences encoding an nsP3 and an nsP4 protein. Accordingly, in some aspects, first polynucleotides of nucleic acid molecules provided herein encode a P123 polyprotein comprising nsP1, nsP2, and nsP3 and a P1234 polyprotein comprising nsP1, nsP2, nsP3, and nsP4 as a result of stop codon readthrough, for example. In other aspects, first polynucleotides of nucleic acid molecules provided herein encode a polyprotein having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, and any number or range in between, identity to a sequence of SEQ ID NO:79. In some embodiments, first polynucleotides of nucleic acid molecules provided herein encode a polyprotein having a sequence of SEQ ID NO:79. Further exemplary polyproteins comprise a sequence of SEQ ID NO:80 or SEQ ID NO:81. In one aspect, nsP2 and nsP3 proteins include mutations. Exemplary mutations include G1309R and S1583G mutations of VEEV proteins. In another aspect, the nsP1, nsP2, and nsP4 proteins are VEEV proteins, and the nsP3 protein is a chikungunya virus (CHIKV) nsP3 protein.

In some aspects, first polynucleotides of nucleic acid molecules provided herein can include a first intergenic region. In some aspects, the first intergenic region is located between a sequence encoding a polyprotein comprising an alphavirus nsP1 protein, an alphavirus nsP2 protein, an alphavirus nsP3 protein, or any combination thereof, and a sequence encoding an alphavirus nsP4 protein. A first intergenic region can comprise any sequence, such as any viral or non-viral sequence. In one aspect, the first intergenic region comprises a viral sequence. In another aspect, the first intergenic region comprises an alphavirus sequence. In yet another aspect, the alphavirus is VEEV. In one aspect, nsP2 and nsP3 proteins include mutations. Exemplary mutations include G1309R and S1583G mutations of VEEV proteins. In another aspect, the nsP1, nsP2, and nsP4 proteins are VEEV proteins, and the nsP3 protein is a chikungunya virus (CHIKV) nsP3 protein.

In some embodiments, the first polynucleotide may comprise a sequence having at least 80% identity to a sequence of SEQ ID NO:72.

In some embodiments, the nucleic acid molecule described herein may further comprise a second polynucleotide comprising a first transgene encoding a first antigenic protein or fragment thereof, wherein the first antigenic protein is a coronavirus protein. In specific embodiments, the antigenic protein may be a SARS-CoV-2 protein. In specific embodiments, the antigenic protein is a SARS-CoV-2 spike glycoprotein. In specific embodiments, the SARS-CoV-2 spike glycoprotein is a wild-type SARS-CoV-2 spike glycoprotein having an amino acid sequence of SEQ ID NO:123.

In some embodiments, the second polynucleotide comprises a sequence having at least 85% identity to a sequence of SEQ ID NO:121 or SEQ ID NO:122.

### 5′ Untranslated Region (5′ UTR)

Nucleic acid molecules provided herein can further comprise untranslated regions (UTRs). Untranslated regions, including 5′ UTRs and 3′ UTRs, for example, can affect RNA stability and/or efficiency of RNA translation, such as translation of cellular and viral mRNAs, for example. 5′ UTRs and 3′ UTRs can also affect stability and translation of viral genomic RNAs and self-replicating RNAs, including virally derived self-replicating RNAs or replicons. Exemplary viral genomic RNAs whose stability and/or efficiency of translation can be affected by 5′ UTRs and 3′ UTRs include the genome nucleic acid of positive-sense RNA viruses. Both genome nucleic acid of positive-sense RNA viruses and self-replicating RNAs, including virally derived self-replicating RNAs or replicons, can be translated upon infection or introduction into a cell.

In some aspects, nucleic acid molecules provided herein further include a 5′ untranslated region (5′ UTR). Any 5′ UTR sequence can be included in nucleic acid molecules provided herein. In some embodiments, nucleic acid molecules provided herein include a viral 5′ UTR. In one aspect, nucleic acid molecules provided herein include a non-viral 5′ UTR. Any non-viral 5′ UTR can be included in nucleic acid molecules provided herein, such as 5′ UTRs of transcripts expressed in any cell or organ, including muscle, skin, subcutaneous tissue, liver, spleen, lymph nodes, antigen-presenting cells, and others. In another aspect, nucleic acid molecules provided herein include a 5′ UTR comprising viral and non-viral sequences. Accordingly, a 5′ UTR included in nucleic acid molecules provided herein can comprise a combination of viral and non-viral 5′ UTR sequences. In some aspects, the 5′ UTR included in nucleic acid molecules provided herein is located upstream of or 5′ of the first polynucleotide that encodes one or more viral replication proteins. In other aspects, the 5′ UTR is located 5′ of or upstream of the first polynucleotide of nucleic acid molecules provided herein that encodes one or more viral replication proteins, and the first polynucleotide is located 5′ of or upstream of the second polynucleotide of nucleic acid molecules provided herein.

In one aspect, the 5′ UTR of nucleic acid molecules provided herein comprises an alphavirus 5′ UTR. A 5′ UTR from any alphavirus can be included in nucleic acid molecules provided herein, including 5′ UTR sequences from Venezuelan Equine Encephalitis Virus (VEEV), Eastern Equine Encephalitis Virus (EEEV), Everglades Virus (EVEV), Mucambo Virus (MUCV), Semliki Forest Virus (SFV), Pixuna Virus (PIXV), Middleburg Virus (MIDV), Chikungunya Virus (CHIKV), O'Nyong-Nyong Virus (ONNV), Ross River Virus (RRV), Barmah Forest Virus (BFV), Getah Virus (GETV), Sagiyama Virus (SAGV), Bebaru Virus (BEBV), Mayaro Virus (MAYV), Una Virus (UNAV), Sindbis Virus (SINV), Aura Virus (AURAV), Whataroa Virus (WHAV), Babanki Virus (BABV), Kyzylagach Virus (KYZV), Western Equine Encephalitis Virus (WEEV), Highland J Virus (HJV), Fort Morgan Virus (FMV), Ndumu Virus (NDUV), Salmonid Alphavirus (SAV), or Buggy Creek Virus (BCRV). In another aspect, the 5′ UTR comprises a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, and any number or range in between, identity to a sequence of SEQ ID NO:73, SEQ ID NO:74, or SEQ ID NO:75. In yet another aspect, the 5′ UTR comprises a sequence of SEQ ID NO:73, SEQ ID NO:74, or SEQ ID NO:75.

In some embodiments, the 5′ UTR comprises a sequence selected from the 5′ UTRs of human IL-6, alanine aminotransferase 1, human apolipoprotein E, human fibrinogen alpha chain, human transthyretin, human haptoglobin, human alpha-1-antichymotrypsin, human antithrombin, human alpha-1-antitrypsin, human albumin, human beta globin, human complement C3, human complement C5, SynK (thylakoid potassium channel protein derived from the cyanobacteria, Synechocystis sp.), mouse beta globin, mouse albumin, and a tobacco etch virus, or fragments of any of the foregoing. Preferably, the 5′ UTR is derived from a tobacco etch virus (TEV). Preferably, an mRNA described herein comprises a 5′ UTR sequence that is derived from a gene expressed by Arabidopsis thaliana. Preferably, the 5′ UTR sequence of a gene expressed by Arabidopsis thaliana is AT1G58420. Examples of 5 UTRs and 3′ UTRs are described in PCT/US2018/035419, the contents of which are herein incorporated by reference. Preferred 5′ UTR sequences comprise SEQ ID NOs: 5-10 and 25-45: as shown in Table 1.

TABLE 1
5' UTR Sequences

| Name | Sequence | Seq ID No.: |
|---|---|---|
| EV | UCAACACAACAUAUACAAAACAAACGAAUCUCAAGCAAUC | SEQ ID NO: 5 |
| | AAGCAUUCUACUUCUAUUGCAGCAAUUUAAAUCAUUUCU | |
| | UUUAAAGCAAAAGCAAUUUUCUGAAAAUUUUCACCAUUU | |
| | ACGAACGAUAG | |
| AT1G58420 | AUUAUUACAUCAAAACAAAAAGCCGCCA | SEQ ID NO: 6 |
| ARC5-2 | CUUAAGGGGGCGCUGCCUACGGAGGUGGCAGCCAUCUCCU | SEQ ID NO: 7 |
| | UCUCGGCAUCAAGCUUACCAUGGUGCCCCAGGCCCUGCUC | |
| | UUGGUCCCGCUGCUGGUGUUCCCCCUCUGCUUCGGCAAGU | |
| | UCCCCAUCUACACCAUCCCCGACAAGCUGGGGCCGUGGAG | |
| | CCCCAUCGACAUCCACCACCUGUCCUGCCCCAACAACCUCG | |
| | UGGUCGAGGACGAGGGCUGCACCAACCUGAGCGGGUUCUC | |
| | CUAC | |
| HCV | UGAGUGUCGU ACAGCCUCCA GGCCCCCCCC | SEQ ID NO: 8 |
| | UCCCGGGAGA GCCAUAGUGG UCUGCGGAAC | |
| | CGGUGAGUAC ACCGGAAUUG CCGGGAAGAC | |
| | UGGGUCCUUU CUUGGAUAAA CCCACUCUAU | |
| | GCCCGGCCAU UUGGGCGUGC CCCCGCAAGA | |
| | CUGCUAGCCG AGUAGUGUUG GGUUGCG | |
| HUMAN | AAUUAUUGGUUAAAGAAGUAUAUUAGUGCUAAUUUCCCU | SEQ ID NO: 9 |
| ALBUMIN | CCGUUUGUCCUAGCUUUUCUCUUCUGUCAACCCCACACGC | |
| | CUUUGGCACA | |
| EMCV | CUCCCUCCCC CCCCCCUAAC GUUACUGGCC | SEQ ID NO: 10 |
| | GAAGCCGCUU GGAAUAAGGC CGGUGUGCGU | |
| | UUGUCUAUAU GUUAUUUUCC ACCAUAUUGC | |
| | CGUCUUUUGG CAAUGUGAGG GCCCGGAAAC | |
| | CUGGCCCUGU CUUCUUGACG AGCAUUCCUA | |
| | GGGGUCUUUC CCCUCUCGCC AAAGGAAUGC | |
| | AAGGUCUGUU GAAUGUCGUG AAGGAAGCAG | |
| | UUCCUCUGGA AGCUUCUUGA AGACAAACAA | |
| | CGUCUGUAGC GACCCUUUGC AGGCAGCGGA | |
| | ACCCCCCACC UGGCGACAGG UGCCUCUGCG | |
| | GCCAAAAGCC ACGUGUAUAA GAUACACCUG | |
| | CAAAGGCGGC ACAACCCCAG UGCCACGUUG | |
| | UGAGUUGGAU AGUUGUGGAA AGAGUCAAAU | |
| | GGCUCUCCUC AAGCGUAUUC AACAAGGGGC | |
| | UGAAGGAUGC CCAGAAGGUA CCCCAUUGUA | |
| | UGGGAUCUGA UCUGGGGCCU CGGUGCACAU | |
| | GCUUUACGUG UGUUUAGUCG AGGUUAAAAA | |
| | ACGUCUAGGC CCCCCGAACC ACGGGGACGU | |
| | GGUUUUCCUU UGAAAAACAC GAUGAUAAU | |
| AT1G67090 | CACAAAGAGUAAAGAAGAACA | SEQ ID NO: 25 |
| AT1G35720 | AACACUAAAAGUAGAAGAAAA | SEQ ID NO: 26 |
| AT5G45900 | CUCAGAAAGAUAAGAUCAGCC | SEQ ID NO: 27 |
| AT5G61250 | AACCAAUCGAAAGAAACCAAA | SEQ ID NO: 28 |
| AT5G46430 | CUCUAAUCACCAGGAGUAAAA | SEQ ID NO: 29 |
| AT5G47110 | GAGAGAGAUCUUAACAAAAAA | SEQ ID NO: 30 |
| AT1G03110 | UGUGUAACAACAACAACAACA | SEQ ID NO: 31 |
| AT3G12380 | CCGCAGUAGGAAGAGAAAGCC | SEQ ID NO: 32 |
| AT5G45910 | AAAAAAAAAAGAAAUCAUAAA | SEQ ID NO: 33 |
| AT1G07260 | GAGAGAAGAAAGAAGAAGACG | SEQ ID NO: 34 |
| AT3G55500 | CAAUUAAAAAUACUUACCAAA | SEQ ID NO: 35 |
| AT3G46230 | GCAAACAGAGUAAGCGAAACG | SEQ ID NO: 36 |
| AT2G36170 | GCGAAGAAGACGAACGCAAAG | SEQ ID NO: 37 |
| AT1G10660 | UUAGGACUGUAUUGACUGGCC | SEQ ID NO: 38 |
| AT4G14340 | AUCAUCGGAAUUCGGAAAAAG | SEQ ID NO: 39 |
| AT1G49310 | AAAACAAAAGUUAAAGCAGAC | SEQ ID NO: 40 |
| AT4G14360 | UUUAUCUCAAAUAAGAAGGCA | SEQ ID NO: 41 |
| AT1G28520 | GGUGGGGAGGUGAGAUUUCUU | SEQ ID NO: 42 |
| AT1G20160 | UGAUUAGGAAACUACAAAGCC | SEQ ID NO: 43 |
| AT5G37370 | CAUUUUUCAAUUUCAUAAAAC | SEQ ID NO: 44 |
| AT4G11320 | UUACUUUUAAGCCCAACAAAA | SEQ ID NO: 45 |
| AT5G40850 | GGCGUGUGUGUGUGUUGUUGA | SEQ ID NO: 46 |
| AT1G06150 | GUGGUGAAGGGGAAGGUUUAG | SEQ ID NO: 47 |
| AT2G26080 | UUGUUUUUUUUUGGUUUGGUU | SEQ ID NO: 48 |

### 3′ Untranslated Region (3′ UTR)

In some aspects, nucleic acid molecules provided herein further include a 3′ untranslated region (3′ UTR). Any 3′ UTR sequence can be included in nucleic acid molecules provided herein. In one aspect, nucleic acid molecules provided herein include a viral 3′ UTR. In another aspect, nucleic acid molecules provided herein include a non-viral 3′ UTR. Any non-viral 3′ UTR can be included in nucleic acid molecules provided herein, such as 3′ UTRs of transcripts expressed in any cell or organ, including muscle, skin, subcutaneous tissue, liver, spleen, lymph nodes, antigen-presenting cells, and others. In some aspects, nucleic acid molecules provided herein include a 3′ UTR comprising viral and non-viral sequences. Accordingly, a 3′ UTR included in nucleic acid molecules provided herein can comprise a combination of viral and non-viral 3′ UTR sequences. In one aspect, the 3′ UTR is located 3′ of or downstream of the second polynucleotide of nucleic acid molecules provided herein that comprises a first transgene encoding a first antigenic protein or a fragment thereof. In another aspect, the 3′ UTR is located 3′ of or downstream of the second polynucleotide of nucleic acid molecules provided herein that comprises a first transgene encoding a first antigenic protein or a fragment thereof, and the second polynucleotide is located 3′ of or downstream of the first polynucleotide of nucleic acid molecules provided herein.

In one aspect, the 3′ UTR of nucleic acid molecules provided herein comprises an alphavirus 3′ UTR. A 3′ UTR from any alphavirus can be included in nucleic acid molecules provided herein, including 3′ UTR sequences from Venezuelan Equine Encephalitis Virus (VEEV), Eastern Equine Encephalitis Virus (EEEV), Everglades Virus (EVEV), Mucambo Virus (MUCV), Semliki Forest Virus (SFV), Pixuna Virus (PIXV), Middleburg Virus (MIDV), Chikungunya Virus (CHIKV), O'Nyong-Nyong Virus (ONNV), Ross River Virus (RRV), Barmah Forest Virus (BFV), Getah Virus (GETV), Sagiyama Virus (SAGV), Bebaru Virus (BEBV), Mayaro Virus (MAYV), Una Virus (UNAV), Sindbis Virus (SINV), Aura Virus (AURAV), Whataroa Virus (WHAV), Babanki Virus (BABV), Kyzylagach Virus (KYZV), Western Equine Encephalitis Virus (WEEV), Highland J Virus (HJV), Fort Morgan Virus (FMV), Ndumu Virus (NDUV), Salmonid Alphavirus (SAV), or Buggy Creek Virus (BCRV). In another aspect, the 3′ UTR comprises a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, and any number or range in between, identity to a sequence of SEQ ID NO:76. In yet another aspect, the 3′ UTR comprises a poly-A sequence. In a further aspect, the 3′ UTR comprises a sequence of SEQ ID NO:76.

In some embodiments, the 3′ UTR comprises a sequence selected from the 3′ UTRs of alanine aminotransferase 1, human apolipoprotein E, human fibrinogen alpha chain, human haptoglobin, human antithrombin, human alpha globin, human beta globin, human complement C3, human growth factor, human hepcidin, MALAT-1, mouse beta globin, mouse albumin, and Xenopus beta globin, or fragments of any of the foregoing. In some embodiments, the 3′ UTR is derived from Xenopus beta globin. Exemplary 3′ UTR sequences include SEQ ID NOs: 16-22 as shown in Table 2.

TABLE 2
3' UTR sequences.

| Name | Sequence | Seq ID No.: |
|---|---|---|
| XBG | CUAGUGACUGACUAGGAUCUGGUUACCACUAAACCAG | SEQ ID NO: 16 |
| | CCUCAAGAACACCCGAAUGGAGUCUCUAAGCUACAUA | |
| | AUACCAACUUACACUUACAAAAUGUUGUCCCCCAAAA | |
| | UGUAGCCAUUCGUAUCUGCUCCUAAUAAAAAGAAAGU | |
| | UUCUUCACAU | |
| HUMAN | UGCAAGGCUGGCCGGAAGCCCUUGCCUGAAAGCAAGA | SEQ ID NO: 17 |
| HAPTOGLOBIN | UUUCAGCCUGGAAGAGGGCAAAGUGGACGGGAGUGG | |
| | ACAGGAGUGGAUGCGAUAAGAUGUGGUUUGAAGCUG | |
| | AUGGGUGCCAGCCCUGCAUUGCUGAGUCAAUCAAUAA | |
| | AGAGCUUUCUUUUGACCCAU | |
| HUMAN | ACGCCGAAGCCUGCAGCCAUGCGACCCCACGCCACCCC | SEQ ID NO: 18 |
| APOLIPOPROTEIN | GUGCCUCCUGCCUCCGCGCAGCCUGCAGCGGGAGACC | |
| E | CUGUCCCCGCCCCAGCCGUCCUCCUGGGGUGGACCCU | |
| | AGUUUAAUAAAGAUUCACCAAGUUUCACGCA | |
| HCV | UAGAGCGGCAAACCCUAGCUACACUCCAUAGCUAGUU | SEQ ID NO: 19 |
| | UCUUUUUUUUUUGUUUUUUUUUUUUUUUUUUUUUUU | |
| | UUUUUUUUUUUUUUUUCCUUUCUUUUCCUUCUUUUU | |
| | UUCCUCUUUUCUUGGUGGCUCCAUCUUAGCCCUAGUC | |
| | ACGGCUAGCUGUGAAAGGUCCGUGAGCCGCAUGACUG | |
| | CAGAGAGUGCCGUAACUGGUCUCUCUGCAGAUCAUGU | |
| MOUSE | ACACAUCACAACCACAACCUUCUCAGGCUACCCUGAG | SEQ ID NO: 20 |
| ALBUMIN | AAAAAAAGACAUGAAGACUCAGGACUCAUCUUUUCUG | |
| | UUGGUGUAAAAUCAACACCCUAAGGAACACAAAUUUC | |
| | UUUAAACAUUUGACUUCUUGUCUCUGUGCUGCAAUUA | |
| | AUAAAAAAUGGAAAGAAUCUAC | |
| HUMAN ALPHA | GCUGGAGCCUCGGUAGCCGUUCCUCCUGCCCGCUGGG | SEQ ID NO: 21 |
| GLOBIN | CCUCCCAACGGGCCCUCCUCCCCUCCUUGCACCGGCCC | |
| | UUCCUGGUCUUUGAAUAAAGUCUGAGUGGGCAGCA | |
| EMCV | UAGUGCAGUCAC UGGCACAACG CGUUGCCCGG | SEQ ID NO: 22 |
| | UAAGCCAAUC GGGUAUACAC GGUCGUCAUA | |
| | CUGCAGACAG GGUUCUUCUA CUUUGCAAGA | |
| | UAGUCUAGAG UAGUAAAAUA AAUAGUAUAAG | |

### Triple Stop Codon

In some embodiments, the self-replicating RNA may comprise a sequence immediately downstream of a coding region (i.e., ORF) that creates a triple stop codon. A triple stop codon is a sequence of three consecutive stop codons. The triple stop codon can ensure total insulation of an expression cassette and may be incorporated to enhance the efficiency of translation. In some embodiments, a self-replicating RNA of the disclosure may comprise a triple combination of any of the sequences UAG, UGA, or UAA immediately downstream of a ORF described herein. The triple combination can be three of the same codons, three different codons, or any other permutation of the three stop codons.

### Translation Enhancers and Kozak Sequences

For translation initiation, proper interactions between ribosomes and mRNAs must be established to determine the exact position of the translation initiation region. However, ribosomes also must dissociate from the translation initiation region to slide toward the downstream sequence during mRNA translation. Translation enhancers upstream from initiation sequences of mRNAs enhance the yields of protein biosynthesis. Several studies have investigated the effects of translation enhancers. In some embodiments, an mRNA described herein comprises a translation enhancer sequence. These translation enhancer sequences enhance the translation efficiency of a self-replicating RNA of the disclosure and thereby provide increased production of the protein encoded by the mRNA. The translation enhancer region may be located in the 5′ or 3′ UTR of an mRNA sequence. Examples of translation enhancer regions include naturally-occurring enhancer regions from the TEV 5′ UTR and the Xenopus beta-globin 3′ UTR. Exemplary 5′ UTR enhancer sequences include but are not limited to those derived from mRNAs encoding human heat shock proteins (HSP) including HSP70-P2, HSP70-M1 HSP72-M2, HSP17.9 and HSP70-P1. Preferred translation enhancer sequences used in accordance with the embodiments of the present disclosure are represented by SEQ ID Nos: 11-15 as shown in Table 3.

TABLE 3
5' UTR Enhancers

| | | Seq |
|---|---|---|
| Name | Sequence | ID No.: |
| HSP70-P2 | GUCAGCUUUCAAACUCUUUGUUUCUUGU | SEQ ID |
| | UUGUUGAUUGAGAAUA | NO: 11 |
| HSP70-M1 | CUCUCGCCUGAGAAAAAAAAUCCACGAA | SEQ ID |
| | CCAAUUUCUCAGCAACCAGCAGCACG | NO: 12 |
| HSP72-M2 | ACCUGUGAGGGUUCGAAGGAAGUAGCAG | SEQ ID |
| | UGUUUUUUGUUCCUAGAGGAAGAG | NO: 13 |
| HSP17.9 | ACACAGAAACAUUCGCAAAAACAAAAUC | SEQ ID |
| | CCAGUAUCAAAAUUCUUCUCUUUUUUUC | NO: 14 |
| | AUAUUUCGCAAAGAC | |
| HSP70-P1 | CAGAAAAAUUUGCUACAUUGUUUCACAA | SEQ ID |
| | ACUUCAAAUAUUAUUCAUUUAUUU | NO: 15 |

In some embodiments, a self-replicating RNA of the disclosure comprises a Kozak sequence. As is understood in the art, a Kozak sequence is a short consensus sequence centered around the translational initiation site of eukaryotic mRNAs that allows for efficient initiation of translation of the mRNA. See, for example, Kozak, Marilyn (1988) Mol. and Cell Biol, 8:2737-2744; Kozak, Marilyn (1991) J. Biol. Chem, 266: 19867-19870; Kozak, Marilyn (1990) Proc Natl. Acad. Sci. USA, 87:8301-8305; and Kozak, Marilyn (1989) J. Cell Biol, 108:229-241. It ensures that a protein is correctly translated from the genetic message, mediating ribosome assembly and translation initiation. The ribosomal translation machinery recognizes the AUG initiation codon in the context of the Kozak sequence. A Kozak sequence may be inserted upstream of the coding sequence for the protein of interest, downstream of a 5′ UTR or inserted upstream of the coding sequence for the protein of interest and downstream of a 5′ UTR. In some embodiments, a self-replicating RNA described herein comprises a Kozak sequence having the amino acid sequence GCCACC (SEQ ID NO: 23). Preferably a self-replicating RNA described herein comprises a partial Kozak sequence “p” having the amino acid sequence GCCA (SEQ ID NO: 24).

### Transgenes

Transgenes included in nucleic acid molecules provided herein can encode an antigenic protein or a fragment thereof. In some embodiments, second polynucleotides of nucleic acid molecules provided herein comprise a first transgene. A first transgene included in second polynucleotides of nucleic acid molecules provided herein can encode a first antigenic protein or a fragment thereof. A transgene included in second polynucleotides of nucleic acid molecules provided herein can comprise a sequence encoding the full amino acid sequence of an antigenic protein or a sequence encoding any suitable portion or fragment of the full amino acid sequence of an antigenic protein. In some embodiments, the antigenic protein is a coronavirus protein.

In another embodiment, the antigenic protein, when administered to a mammalian subject, raises an immune response to a pathogen, such as a coronavirus. In some more particular embodiments, the antigenic protein is expressed on the outer surface of the coronavirus; while in other more particular embodiments, the antigen may be a non-surface antigen, e.g., useful as a T-cell epitope. The immunogen may elicit an immune response against a coronavirus. The immune response may comprise an antibody response (usually including IgG) and/or a cell mediated immune response. The polypeptide immunogen will typically elicit an immune response that recognizes the corresponding coronavirus The immunogen will typically be a surface polypeptide e.g. an envelope glycoprotein, a spike glycoprotein, etc.

In some aspects, the viral protein encoded by transgenes included in nucleic acid molecules provided herein is a coronavirus protein. In some embodiments, the antigenic protein is a SARS-CoV-2 protein.

In one aspect, the antigenic protein is a SARS-CoV-2 spike glycoprotein or a fragment thereof. In another aspect, the SARS-CoV-2 spike glycoprotein is a wild-type SARS-CoV-2 spike glycoprotein. In some aspects, the wild-type SARS-CoV-2 spike glycoprotein has an amino acid sequence of SEQ ID NO: 123. In yet another aspect, the second polynucleotide of nucleic acid molecules provided herein comprises a sequence having at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, and any number or range in between, identity to a sequence of SEQ ID NO:121 or SEQ ID NO:122. In another aspect, the second polynucleotide of nucleic acid molecules provided herein comprises a sequence of SEQ ID NO:121 or SEQ ID NO:122. Accordingly, in some aspects, first transgenes included in second polynucleotides of nucleic acid molecules provided herein comprise a sequence having at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, and any number or range in between, or 100% identity to a sequence of SEQ ID NO:121 or SEQ ID NO:122.

In one aspect, the second polynucleotide of nucleic acid molecules provided herein encodes a wild-type SARS-CoV-2 spike glycoprotein or a fragment thereof. In some aspects, a wild-type SARS-CoV-2 spike glycoprotein comprises a sequence of SEQ ID NO:123. In another aspect, the second polynucleotide of nucleic acid molecules provided herein encodes a SARS-CoV-2 spike protein comprising one or more mutations as compared to a wild-type SARS-CoV-2 spike glycoprotein sequence. Mutations can include substitutions, deletions, insertions, and others. Mutations can be present at any position or at any combination of positions of a SARS-CoV-2 spike glycoprotein. Any number of substitutions, insertions, deletions, or combinations thereof, can be present at any one or more positions of a SARS-CoV-2 spike glycoprotein. As an example, substitutions can include a change of a wild-type amino acid at any position or at any combination of positions to any other amino acid or combination of any other amino acids. Exemplary mutations include mutations at positions 614, 936, 320, 477, 986, 987, or any combination thereof. In one aspect, a SARS-CoV-2 spike glycoprotein or a fragment thereof encoded by transgenes of second polynucleotides included in nucleic acid molecules provided herein includes a D614G mutation, a D936Y mutation, a D936H mutation, a V320G mutation, an S477N mutation, an S477I mutation, an S477T mutation, a K986P mutation, a V987P mutation, or any combination thereof. Additional mutations and variants can be found in the National Bioinformatics Center 2019 Novel Coronavirus Information Database (2019nCoVR), National Genomics Data Center, China National Center for Bioinformation/Beijing Institute of Genomics, Chinese Academy of Science at bigd.big.ac.cn/ncov/variation/annotation. In another aspect, the second polynucleotide includes a transgene encoding a SARS-CoV-2 glycoprotein having at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, and any number or range in between, or 100% identity to a sequence of SEQ ID NO:123.

In some aspects, the second polynucleotide of nucleic acid molecules provided herein comprises at least two transgenes, such as a second coronavirus protein. Any number of transgenes can be included in second polynucleotides of nucleic acid molecules provided herein, such as one, two, three, four, five, six, seven, eight, nine, ten, or more transgenes. In one aspect, the second polynucleotide of nucleic acid molecules provided herein includes a second transgene encoding a second antigenic protein or a fragment thereof or an immunomodulatory protein. In one aspect, the second polynucleotide further comprises an internal ribosomal entry site (IRES), a sequence encoding a 2A peptide, or a combination thereof, located between transgenes. As used herein, the term “2A peptide” refers to a small (generally 18-22 amino acids) sequence that allows for efficient, stoichiometric production of discrete protein products within a single reading frame through a ribosomal skipping event within the 2A peptide sequence. As used herein, the term “internal ribosomal entry site” or “IRES” refers to a nucleotide sequence that allows for the initiation of protein translation of a messenger RNA (mRNA) sequence in the absence of an AUG start codon or without using an AUG start codon. An IRES can be found anywhere in an mRNA sequence, such as at or near the beginning, at or near the middle, or at or near the end of the mRNA sequence, for example.

Any number of transgenes included in second polynucleotides of nucleic acid molecules provided herein can be expressed via any combination of 2A peptide and IRES sequences. For example, a second transgene located 3′ of a first transgene can be expressed via a 2A peptide sequence or via an IRES sequence. As another example, a second transgene located 3′ of a first transgene and a third transgene located 3′ of the second transgene can be expressed via 2A peptide sequences located between the first and second transgenes and the second and third transgenes, via an IRES sequence located between the first and second transgenes and the second and third transgenes, via a 2A peptide sequence located between the first and second transgenes and an IRES located between the second and third transgenes, or via an IRES sequence located between the first and second transgenes and a 2A peptide sequence located between the second and third transgenes. Similar configurations and combinations of 2A peptide and IRES sequences located between transgenes are contemplated for any number of transgenes included in second polynucleotides of nucleic acid molecules provided herein. In addition to expression via 2A peptide and IRES sequences, two or more transgenes included in nucleic acid molecules provided herein can also be expressed from separate subgenomic RNAs.

A second, third, fourth, fifth, sixth, seventh, eighth, ninth, tenth, etc., transgene included in second polynucleotides of nucleic acid molecules provided herein can encode an immunomodulatory protein or a functional fragment or functional variant thereof. Any immunomodulatory protein or a functional fragment or functional variant thereof can be encoded by a transgene included in second polynucleotides.

As used herein, the terms “functional variant” or “functional fragment” refer to a molecule, including a nucleic acid or protein, for example, that comprises a nucleotide and/or amino acid sequence that is altered by one or more nucleotides and/or amino acids compared to the nucleotide and/or amino acid sequences of the parent or reference molecule. For a protein, a functional variant is still able to function in a manner that is similar to the parent molecule. In other words, the modifications in the amino acid and/or nucleotide sequence of the parent molecule do not significantly affect or alter the functional characteristics of the molecule encoded by the nucleotide sequence or containing the amino acid sequence. The functional variant may have conservative sequence modifications including nucleotide and amino acid substitutions, additions and deletions. These modifications can be introduced by standard techniques known in the art, such as site-directed mutagenesis and random PCR-mediated mutagenesis. Functional variants can also include, but are not limited to, derivatives that are substantially similar in primary structural sequence, but which contain, e.g., in vitro or in vivo modifications, chemical and/or biochemical, that are not found in the parent molecule. Such modifications include, inter alia, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI-anchor formation, hydroxylation, iodination, methylation, myristoylation, oxidation, pegylation, proteolytic processing, phosphorylation, prenylation, racemization, selenoylation, sulfation, transfer-RNA-mediated addition of amino acids to proteins such as arginylation, ubiquitination, and the like.

In one aspect, a second transgene included in second polynucleotides of nucleic acid molecules provided herein encodes a cytokine, a chemokine, or an interleukin. Exemplary cytokines include interferons, TNF-α□, TGF-β, G-CSF, and GM-CSF. Exemplary chemokines include CCL3, CCL26, and CXCL7. Exemplary interleukins include IL-I, IL-2, IL-3, IL-4, IL-5, IL-6, IL-7, IL-8, IL-10, IL-12, IL-15, IL-18, IL-21, and IL-23. Any transgene or combination of transgenes encoding any cytokine, chemokine, interleukin, or combinations thereof, can be included in second polynucleotides of nucleic acid molecules provided herein.

In some embodiments, the second transgene encodes a second coronavirus protein.

### DNA and RNA Molecules

Nucleic acid molecules provided herein can be DNA molecules or RNA molecules. It will be appreciated that T present in DNA is substituted with U in RNA, and vice versa. In one aspect, nucleic acid molecules provided herein are DNA molecules. In another aspect, DNA molecules provided herein further comprise a promoter. As used herein, the term “promoter” refers to a regulatory sequence that initiates transcription. A promoter can be operably linked to first and second polynucleotides of nucleic acid molecules provided herein. Generally, promoters included in DNA molecules provided herein include promoters for in vitro transcription (IVT). Any suitable promoter for in vitro transcription can be included in DNA molecules provided herein, such as a T7 promoter, a T3 promoter, an SP6 promoter, and others. In one aspect, DNA molecules provided herein comprise a T7 promoter. In another aspect, the promoter is located 5′ of the 5′ UTR included in DNA molecules provided herein. In yet another aspect, the promoter is a T7 promoter located 5′ of the 5′ UTR included in DNA molecules provided herein. In yet another aspect, the promoter overlaps with the 5′ UTR. A promoter and a 5′ UTR can overlap by about one nucleotide, about two nucleotides, about three nucleotides, about four nucleotides, about five nucleotides, about six nucleotides, about seven nucleotides, about eight nucleotides, about nine nucleotides, about ten nucleotides, about 11 nucleotides, about 12 nucleotides, about 13 nucleotides, about 14 nucleotides, about 15 nucleotides, about 16 nucleotides, about 17 nucleotides, about 18 nucleotides, about 19 nucleotides, about 20 nucleotides, about 21 nucleotides, about 22 nucleotides, about 23 nucleotides, about 24 nucleotides, about 25 nucleotides, about 26 nucleotides, about 27 nucleotides, about 28 nucleotides, about 29 nucleotides, about 30 nucleotides, about 31 nucleotides, about 32 nucleotides, about 33 nucleotides, about 34 nucleotides, about 35 nucleotides, about 36 nucleotides, about 37 nucleotides, about 38 nucleotides, about 39 nucleotides, about 40 nucleotides, about 41 nucleotides, about 42 nucleotides, about 43 nucleotides, about 44 nucleotides, about 45 nucleotides, about 46 nucleotides, about 47 nucleotides, about 48 nucleotides, about 49 nucleotides, about 50 nucleotides, or more nucleotides.

In some aspects, DNA molecules provided herein include a promoter for in vivo transcription. Generally, the promoter for in vivo transcription is an RNA polymerase II (RNA pol II) promoter. Any RNA pol II promoter can be included in DNA molecules provided herein, including constitutive promoters, inducible promoters, and tissue-specific promoters. Exemplary constitutive promoters include a cytomegalovirus (CMV) promoter, an EF1α□promoter, an SV40 promoter, a PGK1 promoter, a Ubc promoter, a human beta actin promoter, a CAG promoter, and others. Any tissue-specific promoter can be included in DNA molecules provided herein. In one aspect, the RNA pol II promoter is a muscle-specific promoter, skin-specific promoter, subcutaneous tissue-specific promoter, liver-specific promoter, spleen-specific promoter, lymph node-specific promoter, or a promoter with any other tissue specificity. DNA molecules provided herein can also include an enhancer. Any enhancer that increases transcription can be included in DNA molecules provided herein.

In some aspects, nucleic acid molecules provided herein are RNA molecules. An RNA molecule provided herein can be generated by in vitro transcription (IVT) of DNA molecules provided herein. In one aspect, RNA molecules provided herein are self-replicating RNA molecules. In another aspect, RNA molecules provided herein further comprise a 5′ cap. Any 5′ cap can be included in RNA molecules provided herein, including 5′ caps having a Cap 1 structure, a Cap 1 (m6A) structure, a Cap 2 structure, a Cap 0 structure, or any combination thereof. In one aspect, RNA molecules provided herein include a 5′ cap having Cap 1 structure. In yet another aspect, RNA molecules provided herein are self-replicating RNA molecules comprising a 5′ cap having a Cap 1 structure. In a further aspect, RNA molecules provided herein comprise a cap having a Cap 1 structure, wherein a m7G is linked via a 5′-5′ triphosphate to the 5′ end of the 5′ UTR. In yet a further aspect, RNA molecules provided herein comprise a cap having a Cap 1 structure, wherein a m7G is linked via a 5′-5′ triphosphate to the 5′ end of the 5′ UTR comprising a sequence of SEQ ID NO:73. Any method of capping can be used, including, but not limited to using a Vaccinia Capping enzyme (New England Biolabs, Ipswich, Mass.) and co-transcriptional capping or capping at or shortly after initiation of in vitro transcription (IVT), by for example, including a capping agent as part of an in vitro transcription (IVT) reaction. (Nuc. Acids Symp. (2009) 53:129).

Provided herein, in some embodiments, are nucleic acid molecules comprising (a) a sequence of SEQ ID NO:10; (b) a sequence of SEQ ID NO:72, SEQ ID NO:73, SEQ ID NO:76, and SEQ ID NO:77, wherein T is substituted with U; (c) a sequence of SEQ ID NO: 124; (d) a sequence of SEQ ID NO:124, wherein T is substituted with U; (e) a sequence of SEQ ID NO:125; or (f) a sequence of SEQ ID NO:125, wherein T is substituted with U. In one aspect, nucleic acid molecules provided herein are RNA molecules. In another aspect, RNA molecules provided herein further comprise a 5′ cap having a Cap 1 structure. Any RNA molecules provided herein can be self-replicating RNA molecules.

Only those mRNAs that carry the Cap structure are active in Cap dependent translation; “decapitation” of mRNA results in an almost complete loss of their template activity for protein synthesis (Nature, 255:33-37, (1975); J. Biol. Chem., vol. 253:5228-5231, (1978); and Proc. Natl. Acad. Sci. USA, 72:1189-1193, (1975)).

Another element of eukaryotic mRNA is the presence of 2′-O-methyl nucleoside residues at transcript position 1 (Cap 1), and in some cases, at transcript positions 1 and 2 (Cap 2). The 2′-O-methylation of mRNA provides higher efficacy of mRNA translation in vivo (Proc. Natl. Acad. Sci. USA, 77:3952-3956 (1980)) and further improves nuclease stability of the 5′-capped mRNA. The mRNA with Cap 1 (and Cap 2) is a distinctive mark that allows cells to recognize the bona fide mRNA 5′ end, and in some instances, to discriminate against transcripts emanating from infectious genetic elements (Nucleic Acid Research 43: 482-492 (2015)).

Some examples of 5′ cap structures and methods for preparing mRNAs comprising the same are given in WO2015/051169A2, WO/2015/061491, US 2018/0273576, and U.S. Pat. Nos. 8,093,367, 8,304,529, and 10,487,105. In some embodiments, the 5′ cap is m7GpppAmpG, which is known in the art. In some embodiments, the 5′ cap is m7GpppG or m7GpppGm, which are known in the art. Structural formulas for embodiments of 5′ cap structures are provided below.

In some embodiments, a self-replicating RNA of the disclosure comprises a 5′ cap having the structure of Formula (Cap I).

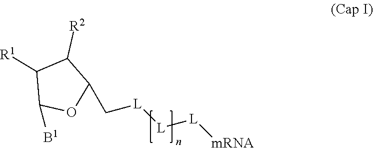

wherein B1 is a natural or modified nucleobase; R¹ and R² are each independently selected from a halogen, OH, and OCH₃; each L is independently selected from the group consisting of phosphate, phophorothioate, and boranophosphate wherein each L is linked by diester bonds; n is 0 or 1, and mRNA represents an mRNA of the present disclosure linked at its 5′ end. In some embodiments B¹ is G, m⁷G, or A. In some embodiments, n is 0. In some embodiments n is 1. In some embodiments, B¹ is A or m⁶A and R¹ is OCH₃; wherein G is guanine, m⁷G is 7-methylguanine, A is adenine, and m⁶A is N⁶-methyladenine.

In some embodiments, a self-replicating RNA of the disclosure comprises a 5′ cap having the structure of Formula (Cap II).

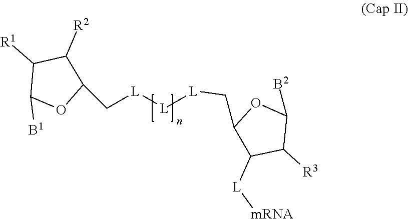

wherein B¹ and B² are each independently a natural or modified nucleobase; R¹, R², and R³ are each independently selected from a halogen, OH, and OCH₃; each L is independently selected from the group consisting of phosphate, phophorothioate, and boranophosphate wherein each L is linked by diester bonds; mRNA represents an mRNA of the present disclosure linked at its 5′ end; and n is 0 or 1. In some embodiments B¹ is G, m⁷G, or A. In some embodiments, n is 0. In some embodiments, n is 1. In some embodiments, B¹ is A or m⁶A and R¹ is OCH₃; wherein G is guanine, m⁷G is 7-methylguanine, A is adenine, and m⁶A is N⁶-methyladenine.

In some embodiments, a self-replicating RNA of the disclosure comprises a 5′ cap having the structure of Formula (Cap III).

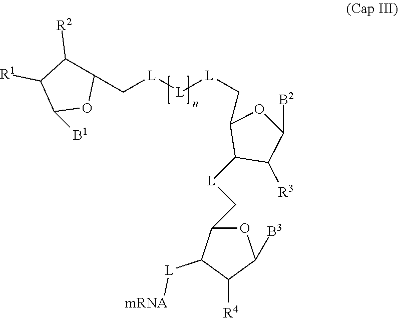

wherein B1, B2, and B3 are each independently a natural or modified nucleobase; R1, R2, R3, and R4 are each independently selected from a halogen, OH, and OCH3; each L is independently selected from the group consisting of phosphate, phosphorothioate, and boranophosphate wherein each L is linked by diester bonds; mRNA represents an mRNA of the present disclosure linked at its 5′ end; and n is 0 or 1. In some embodiments, at least one of R1, R2, R3, and R4 is OH. In some embodiments B1 is G, m7G, or A. In some embodiments, B1 is A or m6A and R1 is OCH3; wherein G is guanine, m7G is 7-methylguanine, A is adenine, and m6A is N6-methyladenine. In some embodiments, n is 1.

In some embodiments, a self-replicating RNA of the disclosure comprises a m7GpppG 5′ cap analog having the structure of Formula (Cap IV).

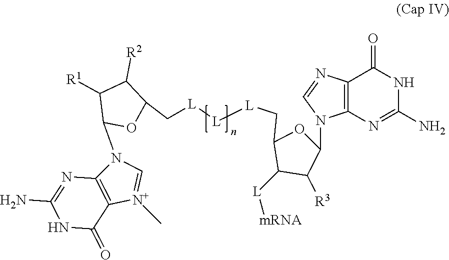

wherein, R¹, R², and R³ are each independently selected from a halogen, OH, and OCH₃; each L is independently selected from the group consisting of phosphate, phosphorothioate, and boranophosphate wherein each L is linked by diester bonds; mRNA represents an mRNA of the present disclosure linked at its 5′ end; n is 0 or 1. In some embodiments, at least one of R¹, R², and R³ is OH. In some embodiments, the 5′ cap is m⁷GpppG wherein R¹, R², and R³ are each OH, n is 1, and each L is a phosphate. In some embodiments, n is 1. In some embodiments, the 5′ cap is m7GpppGm, wherein R¹ and R² are each OH, R³ is OCH₃, each L is a phosphate, mRNA is the mRNA encoding an enzyme having OTC activity linked at its 5′ end, and n is 1.

In some embodiments, a self-replicating RNA of the disclosure comprises a m7Gpppm7G 5′ cap analog having the structure of Formula (Cap V).

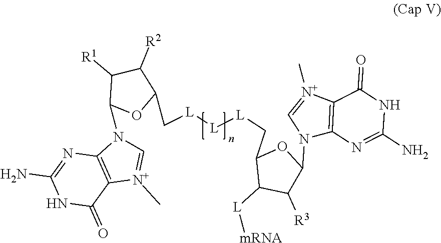

wherein, R¹, R², and R³ are each independently selected from a halogen, OH, and OCH₃; each L is independently selected from the group consisting of phosphate, phosphorothioate, and boranophosphate wherein each L is linked by diester bonds; mRNA represents an mRNA of the present disclosure linked at its 5′ end; and n is 0 or 1. In some embodiments, at least one of R¹, R², and R³ is OH. In some embodiments, n is 1.

In some embodiments, a self-replicating RNA of the disclosure comprises a m7Gpppm7GpN, 5′ cap analog, wherein N is a natural or modified nucleotide, the 5′ cap analog having the structure of Formula (Cap VI).

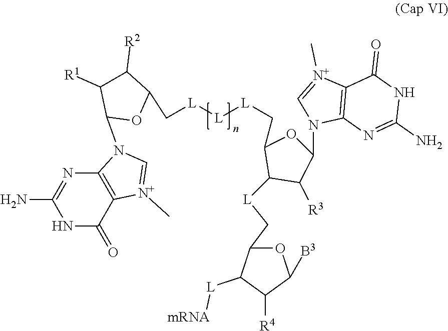

wherein B³ is a natural or modified nucleobase; R¹, R², R³, and R⁴ are each independently selected from a halogen, OH, and OCH₃; each L is independently selected from the group consisting of phosphate, phosphorothioate, and boranophosphate wherein each L is linked by diester bonds; mRNA represents an mRNA of the present disclosure linked at its 5′ end; and n is 0 or 3. In some embodiments, at least one of R¹, R², R³, and R⁴ is OH. In some embodiments B¹ is G, m⁷G, or A. In some embodiments, B¹ is A or m⁶A and R¹ is OCH₃; wherein G is guanine, m⁷G is 7-methylguanine, A is adenine, and m⁶A is N⁶-methyladenine. In some embodiments, n is 1.

In some embodiments, a self-replicating RNA of the disclosure comprises a m7Gpppm7GpG 5′ cap analog having the structure of Formula (Cap VII).

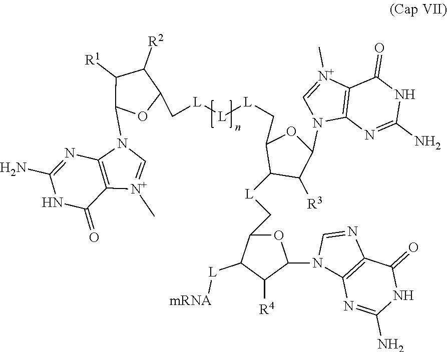

wherein, R¹, R², R³, and R⁴ are each independently selected from a halogen, OH, and OCH₃; each L is independently selected from the group consisting of phosphate, phosphorothioate, and boranophosphate wherein each L is linked by diester bonds; mRNA represents an mRNA of the present disclosure linked at its 5′ end; and n is 0 or 1. In some embodiments, at least one of R¹, R², R³, and R⁴ is OH. In some embodiments, n is 1.

In some embodiments, a self-replicating RNA of the disclosure comprises a m7Gpppm7Gpm7G 5′ cap analog having the structure of Formula (Cap VIII).

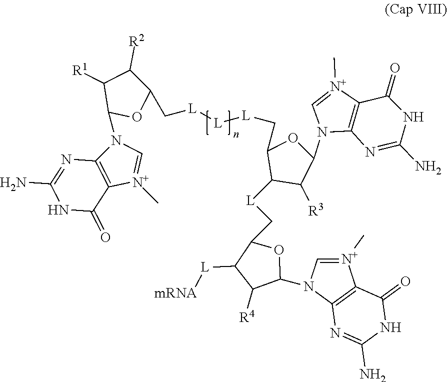

wherein, R¹, R², R³, and R⁴ are each independently selected from a halogen, OH, and OCH₃; each L is independently selected from the group consisting of phosphate, phosphorothioate, and boranophosphate wherein each L is linked by diester bonds; mRNA represents an mRNA of the present disclosure linked at its 5′ end; n is 0 or 1. In some embodiments, at least one of R¹, R², R³, and R⁴ is OH. In some embodiments, n is 1.

In some embodiments, a self-replicating RNA of the disclosure comprises a m7GpppA 5′ cap analog having the structure of Formula (Cap IX).

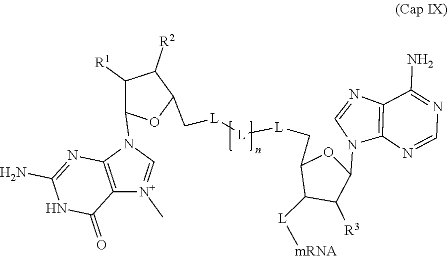

wherein, R¹, R², and R³ are each independently selected from a halogen, OH, and OCH₃; each L is independently selected from the group consisting of phosphate, phosphorothioate, and boranophosphate wherein each L is linked by diester bonds; mRNA represents an mRNA of the present disclosure linked at its 5′ end; and n is 0 or 1. In some embodiments, at least one of R¹, R², and R³ is OH. In some embodiments, n is 1.

In some embodiments, a self-replicating RNA of the disclosure comprises a m7GpppApN 5′ cap analog, wherein N is a natural or modified nucleotide, and the 5′ cap has the structure of Formula (Cap X).

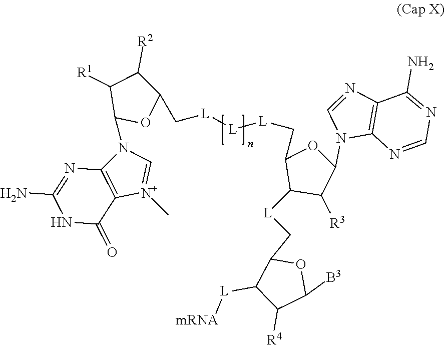

wherein B³ is a natural or modified nucleobase; R¹, R², R³, and R⁴ are each independently selected from a halogen, OH, and OCH₃; each L is independently selected from the group consisting of phosphate, phosphorothioate, and boranophosphate wherein each L is linked by diester bonds; mRNA represents an mRNA of the present disclosure linked at its 5′ end; and n is 0 or 1. In some embodiments, at least one of R¹, R², R³, and R⁴ is OH. In some embodiments B³ is G, m⁷G, A or m⁶A; wherein G is guanine, m⁷G is 7-methylguanine, A is adenine, and m⁶A is N⁶-methyladenine. In some embodiments, n is 1.

In some embodiments, a self-replicating RNA of the disclosure comprises a m7GpppAmpG 5′ cap analog having the structure of Formula (Cap XI).

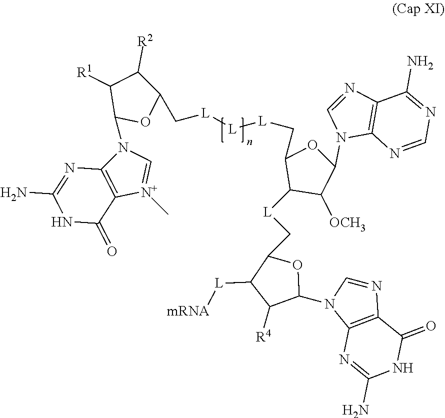

wherein, R¹, R², and R⁴ are each independently selected from a halogen, OH, and OCH₃; each L is independently selected from the group consisting of phosphate, phosphorothioate, and boranophosphate wherein each L is linked by diester bonds; mRNA represents an mRNA of the present disclosure linked at its 5′ end; and n is 0 or 1. In some embodiments, at least one of R¹, R², and R⁴ is OH. In some embodiments, the compound of Formula Cap XI is m⁷GpppAmpG, wherein R¹, R², and R⁴ are each OH, n is 1, and each L is a phosphate linkage. In some embodiments, n is 1.

In some embodiments, a self-replicating RNA of the disclosure comprises a m7GpppApm7G 5′ cap analog having the structure of Formula (Cap XII).

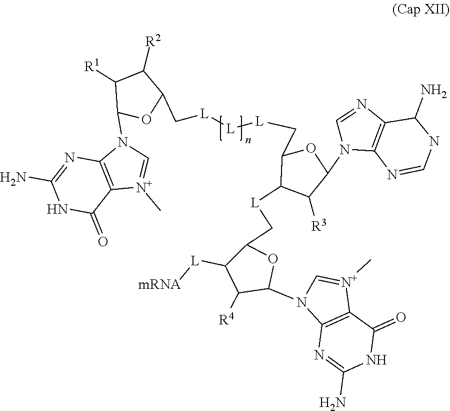

wherein, R¹, R², R³, and R⁴ are each independently selected from a halogen, OH, and OCH₃; each L is independently selected from the group consisting of phosphate, phosphorothioate, and boranophosphate wherein each L is linked by diester bonds; mRNA represents an mRNA of the present disclosure linked at its 5′ end; and n is 0 or 1. In some embodiments, at least one of R¹, R², R³, and R⁴ is OH. In some embodiments, n is 1.

In some embodiments, a self-replicating RNA of the disclosure comprises a m7GpppApm7G 5′ cap analog having the structure of Formula (Cap XIII).

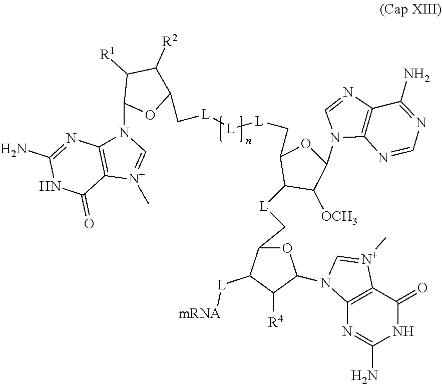

wherein, R¹, R², and R⁴ are each independently selected from a halogen, OH, and OCH₃; each L is independently selected from the group consisting of phosphate, phosphorothioate, and boranophosphate wherein each L is linked by diester bonds; mRNA represents an mRNA of the present disclosure linked at its 5′ end; and n is 0 or 1. In some embodiments, at least one of R¹, R², and R⁴ is OH. In some embodiments, n is 1.

### Poly-Adenine (Poly-A) Tail

Polyadenylation is the addition of a poly(A) tail, a chain of adenine nucleotides usually about 100-120 monomers in length, to a mRNA. In eukaryotes, polyadenylation is part of the process that produces mature mRNA for translation and begins as the transcription of a gene terminates. The 3′-most segment of a newly made pre-mRNA is first cleaved off by a set of proteins; these proteins then synthesize the poly(A) tail at the 3′ end. The poly(A) tail is important for the nuclear export, translation, and stability of mRNA. The tail is shortened over time, and, when it is short enough, the mRNA is enzymatically degraded. However, in a few cell types, mRNAs with short poly(A) tails are stored for later activation by re-polyadenylation in the cytosol.

Preferably, a self-replicating RNA of the disclosure comprises a 3′ tail region, which can serve to protect the RNA from exonuclease degradation. The tail region may be a 3′poly(A) and/or 3′poly(C) region. Preferably, the tail region is a 3′ poly(A) tail. As used herein a “3′ poly(A) tail” is a polymer of sequential adenine nucleotides that can range in size from, for example: 10 to 250 sequential adenine nucleotides; 60-125 sequential adenine nucleotides, 90-125 sequential adenine nucleotides, 95-125 sequential adenine nucleotides, 95-121 sequential adenine nucleotides, 100 to 121 sequential adenine nucleotides, 110-121 sequential adenine nucleotides; 112-121 sequential adenine nucleotides; 114-121 adenine sequential nucleotides; or 115 to 121 sequential adenine nucleotides. Preferably, a 3′ poly(A) tail as described herein comprise 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, or 125 sequential adenine nucleotides. 3′ Poly(A) tails can be added using a variety of methods known in the art, e.g., using poly(A) polymerase to add tails to synthetic or in vitro transcribed RNA. Other methods include the use of a transcription vector to encode poly(A) tails or the use of a ligase (e.g., via splint ligation using a T4 RNA ligase and/or T4 DNA ligase), wherein poly(A) may be ligated to the 3′ end of a sense RNA. In some embodiments, a combination of any of the above methods is utilized.

### Design and Synthesis of Self-Replicating RNA

The constructs for exemplary self-replicating RNA sequences of the present disclosure are provided in Tables 4-5.

TABLE 4
Comparison of STARR ™ self-replicating RNA of the
disclosure with comparative self-replicating RNA as described

| | | Sequence | |
|---|---|---|---|
| Construct | Position | Type | Sequence |
| STARR ™ | 5' UTR | nucleotide | ATGGGCGGCGCATGAGAGAAGCCCAGACCAATTACCT |
| (SEQ ID | | | ACCCAAA |
| NO: 49) | | | |
| STARR ™ | non- | nucleotide | ATGGAGAAAGTTCACGTTGACATCGAGGAAGACAGCC |
| (SEQ ID | structural | | CATTCCTCAGAGCTTTGCAGCGGAGCTTCCCGCAGTTT |
| NO: 50) | gene ORF | | GAGGTAGAAGCCAAGCAGGTCACTGATAATGACCATG |
| | | | CTAATGCCAGAGCGTTTTCGCATCTGGCTTCAAAACTG |
| | | | ATCGAAACGGAGGTGGACCCATCCGACACGATCCTTG |
| | | | ACATTGGAAGTGCGCCCGCCCGCAGAATGTATTCTAA |
| | | | GCACAAGTATCOATTGTATCTGTCCGATGAGATGTGCGG |
| | | | AAGATCCGGACAGATTGTATAAGTATGCAACTAAGCT |
| | | | GAAGAAAAACTGTAAGGAAATAACTGATAAGGAATTG |
| | | | GACAAGAAAATGAAGGAGCTGGCCGCCGTCATGAGCG |
| | | | ACCCTGACCTGGAAACTGAGACTATGTGCCTCCACGA |
| | | | CGACGAGTCGTGTCGCTACGAAGGGCAAGTCGCTGTT |
| | | | TACCAGGATGTATACGCCGTCGACGGCCCCACCAGCC |
| | | | TGTACCACCAGGCCAACAAGGGCGTGAGGGTGGCCTA |
| | | | CTGGATCGGCTTCGACACCACACCCTTCATGTTCAAGA |
| | | | ACCTGGCCGGCGCCTACCCCAGCTACAGCACCAACTG |
| | | | GGCCGACGAGACCGTGCTGACCGCCAGGAACATCGGC |
| | | | CTGTGCAGCAGCGACGTGATGGAGAGGAGCCGGAGAG |
| | | | GCATGAGCATCCTGAGGAAGAAATACCTGAAGCCCAG |
| | | | CAACAACGTGCTGTTCAGCGTGGGCAGCACCATCTAC |
| | | | CACGAGAAGAGGGACCTGCTCAGGAGCTGGCACCTGC |
| | | | CCAGCGTGTTCCACCTGAGGGGCAAGCAGAACTACAC |
| | | | CTGCAGGTGCGAGACCATCGTGAGCTGCGACGGCTAC |
| | | | GTGGTGAAGAGGATCGCCATCAGCCCCGGCCTGTACG |
| | | | GCAAGCCCAGCGGCTACGCCGCTACAATGCACAGGGA |
| | | | GGGCTTCCTGTGCTGCAAGGTGACCGACACCCTGAAC |
| | | | GGCGAGAGGGTGAGCTTCCCCGTGTGCACCTACGTGC |
| | | | CCGCCACCCTGTGCGACCAGATGACCGGCATCCTGGC |
| | | | CACCGACGTGAGCGCCGACGACGCCCAGAAGCTGCTC |
| | | | GTGGGCCTGAACCAGAGGATCGTGGTCAACGGCAGGA |
| | | | CCCAGAGGAACACCAACACAATGAAGAACTACCTGCT |
| | | | GCCCGTGGTGGCCCAGGCTTTCGCCAGGTGGGCCAAG |
| | | | GAGTACAAGGAGGACCAGGAAGACGAGAGGCCCCTG |
| | | | GGCCTGAGGGACAGGCAGCTGGTGATGGGCTGCTGCT |
| | | | GGGCCTTCAGGCGGCACAAGATCACCAGCATCTACAA |
| | | | GAGGCCCGACACCCAGACCATCATCAAGGTGAACAGC |
| | | | GACTTCCACAGCTTCGTGCTGCCCAGGATCGGCAGCA |
| | | | ACACCCTGGAGATCGGCCTGAGGACCCGGATCAGGAA |
| | | | GATGCTGGAGGAACACAAGGAGCCCAGCCCACTGATC |
| | | | ACCGCCGAGGACGTGCAGGAGGCCAAGTGCGCTGCCG |
| | | | ACGAGGCCAAGGAGGTGAGGGAGGCCGAGGAACTGA |
| | | | GGGCCGCCCTGCCACCCCTGGCTGCCGACGTGGAGGA |
| | | | ACCCACCCTGGAAGCCGACGTGGACCTGATGCTGCAG |
| | | | GAGGCCGGCGCCGGAAGCGTGGAGACACCCAGGGGC |
| | | | CTGATCAAGGTGACCAGCTACGACGGCGAGGACAAGA |
| | | | TCGGCAGCTACGCCGTGCTGAGCCCACAGGCCGTGCT |
| | | | GAAGTCCGAGAAGCTGAGCTGCATCCACCCACTGGCC |
| | | | GAGCAGGTGATCGTGATCACCCACAGCGGCAGGAAGG |
| | | | GCAGGTACGCCGTGGAGCCCTACCACGGCAAGGTGGT |
| | | | CGTGCCCGAGGGCCACGCCATCCCCGTGCAGGACTTC |
| | | | CAGGCCCTGAGCGAGAGCGCCACCATCGTGTACAACG |
| | | | AGAGGGAGTTCGTGAACAGGTACCTGCACCATATCGC |
| | | | CACCCACGGCGGAGCCCTGAACACCGACGAGGAATAC |
| | | | TACAAGACCGTGAAGCCCAGCGAGCACGACGGCGAGT |
| | | | ACCTGTACGACATCGACAGGAAGCAGTGCGTGAAGAA |
| | | | AGAGCTGGTGACCGGCCTGGGACTGACCGGCGAGCTG |
| | | | GTGGACCCACCCTTCCACGAGTTCGCCTACGAGAGCCT |
| | | | GAGGACCAGACCCGCCGCTCCCTACCAGGTGCCCACC |
| | | | ATCGGCGTGTACGGCGTGCCCGGCAGCGGAAAGAGCG |
| | | | GCATCATCAAGAGCGCCGTGACCAAGAAAGACCTGGT |
| | | | GGTCAGCGCCAAGAAAGAGAACTGCGCCGAGATCATC |
| | | | AGGGACGTGAAGAAGATGAAAGGCCTGGACGTGAAC |
| | | | GCGCGCACCGTGGACAGCGTGCTGCTGAACGGCTGCA |
| | | | AGCACCCCGTGGAGACCCTGTACATCGACGAGGCCTT |
| | | | CGCTTGCCACGCCGGCACCCTGAGGGCCCTGATCGCC |
| | | | ATCATCAGGCCCAAGAAAGCCGTGCTGTGCGGCGACC |
| | | | CCAAGCAGTGCGGCTTCTTCAACATGATGTGCCTGAAG |
| | | | GTGCACTTCAACCACGAGATCTGCACCCAGGTGTTCCA |
| | | | CAAGAGCATCAGCAGGCGGTGCACCAAGAGCGTGACC |
| | | | AGCGTCGTGAGCACCCTGTTCTACGACAAGAAAATGA |
| | | | GGACCACCAACCCCAAGGAGACCAAAATCGTGATCGA |
| | | | CACCACAGGCAGCACCAAGCCCAAGCAGGACGACCTG |
| | | | ATCCTGACCTGCTTCAGGGGCTGGGTGAAGCAGCTGC |
| | | | AGATCGACTACAAGGGCAACGAGATCATGACCGCCGC |
| | | | TGCCAGCCAGGGCCTGACCAGGAAGGGCGTGTACGCC |
| | | | GTGAGGTACAAGGTGAACGAGAACCCACTGTACGCTC |
| | | | CCACCAGCGAGCACGTGAACGTGCTGCTGACCAGGAC |
| | | | CGAGGACAGGATCGTGTGGAAGACCCTGGCCGGCGAC |
| | | | CCCTGGATCAAGACCCTGACCGCCAAGTACCCCGGCA |
| | | | ACTTCACCGCCACCATCGAAGAGTGGCAGGCCGAGCA |
| | | | CGACGCCATCATGAGGCACATCCTGGAGAGGCCCGAC |
| | | | CCCACCGACGTGTTCCAGAACAAGGCCAACGTGTGCT |
| | | | GGGCCAAGGCCCTGGTGCCCGTGCTGAAGACCGCCGG |
| | | | CATCGACATGACCACAGAGCAGTGGAACACCGTGGAC |
| | | | TACTTCGAGACCGACAAGGCCCACAGCGCCGAGATCG |
| | | | TGCTGAACCAGCTGTGCGTGAGGTTCTTCGGCCTGGAC |
| | | | CTGGACAGCGGCCTGTTCAGCGCCCCCACCGTGCCACT |
| | | | GAGCATCAGGAACAACCACTGGGACAACAGCCCCAGC |
| | | | CCAAACATGTACGGCCTGAACAAGGAGGTGGTCAGGC |
| | | | AGCTGAGCAGGCGGTACCCACAGCTGCCCAGGGCCGT |
| | | | GGCCACCGGCAGGGTGTACGACATGAACACCGGCACC |
| | | | CTGAGGAACTACGACCCCAGGATCAACCTGGTGCCCG |
| | | | TGAACAGGCGGCTGCCCCACGCCCTGGTGCTGCACCA |
| | | | CAACGAGCACCCACAGAGCGACTTCAGCTCCTTCGTG |
| | | | AGCAAGCTGAAAGGCAGGACCGTGCTGGTCGTGGGCG |
| | | | AGAAGCTGAGCGTGCCCGGCAAGATGGTGGACTGGCT |
| | | | GAGCGACAGGCCCGAGGCCACCTTCCGGGCCAGGCTG |
| | | | GACCTCGGCATCCCCGGCGACGTGCCCAAGTACGACA |
| | | | TCATCTTCGTGAACGTCAGGACCCCATACAAGTACCAC |
| | | | CATTACCAGCAGTGCGAGGACCACGCCATCAAGCTGA |
| | | | GCATGCTGACCAAGAAGGCCTGCCTGCACCTGAACCC |
| | | | CGGAGGCACCTGCGTGAGCATCGGCTACGGCTACGCC |
| | | | GACAGGGCCAGCGAGAGCATCATTGGCGCCATCGCCA |
| | | | GGCTGTTCAAGTTCAGCAGGGTGTGCAAACCCAAGAG |
| | | | CAGCCTGGAGGAAACCGAGGTGCTGTTCGTGTTCATC |
| | | | GGCTACGACCGGAAGGCCAGGACCCACAACCCCTACA |
| | | | AGCTGAGCAGCACCCTGACAAACATCTACACCGGCAG |
| | | | CAGGCTGCACGAGGCCGGCTGCGCCCCCAGCTACCAC |
| | | | GTGGTCAGGGGCGATATCGCCACCGCCACCGAGGGCG |
| | | | TGATCATCAACGCTGCCAACAGCAAGGGCCAGCCCGG |
| | | | AGGCGGAGTGTGCGGCGCCCTGTACAAGAAGTTCCCC |
| | | | GAGAGCTTCGACCTGCAGCCCATCGAGGTGGGCAAGG |
| | | | CCAGGCTGGTGAAGGGCGCCGCTAAGCACATCATCCA |
| | | | CGCCGTGGGCCCCAACTTCAACAAGGTGAGCGAGGTG |
| | | | GAAGGCGACAAGCAGCTGGCCGAAGCCTACGAGAGC |
| | | | ATCGCCAAGATCGTGAACGACAATAACTACAAGAGCG |
| | | | TGGCCATCCCACTGCTCAGCACCGGCATCTTCAGCGGC |
| | | | AACAAGGACAGGCTGACCCAGAGCCTGAACCACCTGC |
| | | | TCACCGCCCTGGACACCACCGATGCCGACGTGGCCAT |
| | | | CTACTGCAGGGACAAGAAGTGGGAGATGACCCTGAAG |
| | | | GAGGCCGTGGCCAGGCGGGAGGCCGTGGAAGAGATCT |
| | | | GCATCAGCGACGACTCCAGCGTGACCGAGCCCGACGC |
| | | | CGAGCTGGTGAGGGTGCACCCCAAGAGCTCCCTGGCC |
| | | | GGCAGGAAGGGCTACAGCACCAGCGACGGCAAGACCT |
| | | | TCAGCTACCTGGAGGGCACCAAGTTCCACCAGGCCGC |
| | | | TAAGGACATCGCCGAGATCAACGCTATGTGGCCCGTG |
| | | | GCCACCGAGGCCAACGAGCAGGTGTGCATGTACATCC |
| | | | TGGGCGAGAGCATGTCCAGCATCAGGAGCAAGTGCCC |
| | | | CGTGGAGGAAAGCGAGGCCAGCACACCACCCAGCACC |
| | | | CTGCCCTGCCTGTGCATCCACGCTATGACACCCGAGAG |
| | | | GGTGCAGCGGCTGAAGGCCAGCAGGCCCGAGCAGATC |
| | | | ACCGTGTGCAGCTCCTTCCCACTGCCCAAGTACAGGAT |
| | | | CACCGGCGTGCAGAAGATCCAGTGCAGCCAGCCCATC |
| | | | CTGTTCAGCCCAAAGGTGCCCGCCTACATCCACCCCAG |
| | | | GAAGTACCTGGTGGAGACCCCACCCGTGGACGAGACA |
| | | | CCCGAGCCAAGCGCCGAGAACCAGAGCACCGAGGGC |
| | | | ACACCCGAGCAGCCACCCCTGATCACCGAGGACGAGA |
| | | | CAAGGACCCGGACCCCAGAGCCCATCATTATCGAGGA |
| | | | AGAGGAAGAGGACAGCATCAGCCTGCTGAGCGACGGC |
| | | | CCCACCCACCAGGTGCTGCAGGTGGAGGCCGACATCC |
| | | | ACGGCCCACCCAGCGTGTCCAGCTCCAGCTGGAGCAT |
| | | | CCCACACGCCAGCGACTTCGACGTGGACAGCCTGAGC |
| | | | ATCCTGGACACCCTGGAGGGCGCCAGCGTGACCTCCG |
| | | | GCGCCACCAGCGCCGAGACCAACAGCTACTTCGCCAA |
| | | | GAGCATGGAGTTCCTGGCCAGGCCCGTGCCAGCTCCC |
| | | | AGGACCGTGTTCAGGAACCCACCCCACCCAGCTCCCA |
| | | | GGACCAGGACCCCAAGCCTGGCTCCCAGCAGGGCCTG |
| | | | CAGCAGGACCAGCCTGGTGAGCACCCCACCCGGCGTG |
| | | | AACAGGGTGATCACCAGGGAGGAACTGGAGGCCCTGA |
| | | | CACCCAGCAGGACCCCCAGCAGGTCCGTGAGCAGGAC |
| | | | TAGTCTGGTGTCCAACCCACCCGGCGTGAACAGGGTG |
| | | | ATCACCAGGGAGGAATTCGAGGCCTTCGTGGCCCAGC |
| | | | AACAGAGACGGTTCGACGCCGGCGCCTACATCTTCAG |
| | | | CAGCGACACCGGCCAGGGACACCTGCAGCAAAAGAGC |
| | | | GTGAGGCAGACCGTGCTGAGCGAGGTGGTGCTGGAGA |
| | | | GGACCGAGCTGGAAATCAGCTACGCCCCCAGGCTGGA |
| | | | CCAGGAGAAGGAGGAACTGCTCAGGAAGAAACTGCA |
| | | | GCTGAACCCCACCCCAGCCAACAGGAGCAGGTACCAG |
| | | | AGCAGGAAGGTGGAGAACATGAAGGCCATCACCGCCA |
| | | | GGCGGATCCTGCAGGGCCTGGGACACTACCTGAAGGC |
| | | | CGAGGGCAAGGTGGAGTGCTACAGGACCCTGCACCCC |
| | | | GTGCCACTGTACAGCTCCAGCGTGAACAGGGCCTTCTC |
| | | | CAGCCCCAAGGTGGCCGTGGAGGCCTGCAACGCTATG |
| | | | CTGAAGGAGAACTTCCCCACCGTGGCCAGCTACTGCA |
| | | | TCATCCCCGAGTACGACGCCTACCTGGACATGGTGGA |
| | | | CGGCGCCAGCTGCTGCCTGGACACCGCCAGCTTCTGCC |
| | | | CCGCCAAGCTGAGGAGCTTCCCCAAGAAACACAGCTA |
| | | | CCTGGAGCCCACCATCAGGAGCGCCGTGCCCAGCGCC |
| | | | ATCCAGAACACCCTGCAGAACGTGCTGGCCGCTGCCA |
| | | | CCAAGAGGAACTGCAACGTGACCCAGATGAGGGAGCT |
| | | | GCCCGTGCTGGACAGCGCTGCCTTCAACGTGGAGTGCT |
| | | | TCAAGAAATACGCCTGCAACAACGAGTACTGGGAGAC |
| | | | CTTCAAGGAGAACCCCATCAGGCTGACCGAAGAGAAC |
| | | | GTGGTGAACTACATCACCAAGCTGAAGGGCCCCAAGG |
| | | | CCGCTGCCCTGTTCGCTAAGACCCACAACCTGAACATG |
| | | | CTGCAGGACATCCCAATGGACAGGTTCGTGATGGACC |
| | | | TGAAGAGGGACGTGAAGGTGACACCCGGCACCAAGCA |
| | | | CACCGAGGAGAGGCCCAAGGTGCAGGTGATCCAGGCC |
| | | | GCTGACCCACTGGCCACCGCCTACCTGTGCGGCATCCA |
| | | | CAGGGAGCTGGTGAGGCGGCTGAACGCCGTGCTGCTG |
| | | | CCCAACATCCACACCCTGTTCGACATGAGCGCCGAGG |
| | | | ACTTCGACGCCATCATCGCCGAGCACTTCCAGCCCGGC |
| | | | GACTGCGTGCTGGAGACCGACATCGCCAGCTTCGACA |
| | | | AGAGCGAGGATGACGCTATGGCCCTGACCGCTCTGAT |
| | | | GATCCTGGAGGACCTGGGCGTGGACGCCGAGCTGCTC |
| | | | ACCCTGATCGAGGCTGCCTTCGGCGAGATCAGCTCCAT |
| | | | CCACCTGCCCACCAAGACCAAGTTCAAGTTCGGCGCT |
| | | | ATGATGAAAAGCGGAATGTTCCTGACCCTGTTCGTGA |
| | | | ACACCGTGATCAACATTGTGATCGCCAGCAGGGTGCT |
| | | | GCGGGAGAGGCTGACCGGCAGCCCCTGCGCTGCCTTC |
| | | | ATCGGCGACGACAACATCGTGAAGGGCGTGAAAAGCG |
| | | | ACAAGCTGATGGCCGACAGGTGCGCCACCTGGCTGAA |
| | | | CATGGAGGTGAAGATCATCGACGCCGTGGTGGGCGAG |
| | | | AAGGCCCCCTACTTCTGCGGCGGATTCATCCTGTGCGA |
| | | | CAGCGTGACCGGCACCGCCTGCAGGGTGGCCGACCCC |
| | | | CTGAAGAGGCTGTTCAAGCTGGGCAAGCCACTGGCCG |
| | | | CTGACGATGAGCACGACGATGACAGGCGGAGGGCCCT |
| | | | GCACGAGGAAAGCACCAGGTGGAACAGGGTGGGCAT |
| | | | CCTGAGCGAGCTGTGCAAGGCCGTGGAGAGCAGGTAC |
| | | | GAGACCGTGGGCACCAGCATCATCGTGATGGCTATGA |
| | | | CCACACTGGCCAGCTCCGTCAAGAGCTTCTCCTACCTG |
| | | | AGGGGGGCCCCTATAACTCTCTACGGCTAA |
| STARR ™ | non- | amino acid | MEKVHVDIEEDSPFLRALQRSFPQFEVEAKQVTDNDHAN |
| (SEQ ID | structural | | ARAFSHLASKLIETEVDPSDTILDIGSAPARRMYSKHKYH |
| NO: 51) | gene ORF | | CICPMRCAEDPDRLYKYATKLKKNCKEITDKELDKKMK |
| | | | ELAAVMSDPDLETETMCLHDDESCRYEGQVAVYQDVY |
| | | | AVDGPTSLYHQANKGVRVAYWIGFDTTPFMFKNLAGAY |
| | | | PSYSTNWADETVLTARNIGLCSSDVMERSRRGMSILRKK |
| | | | YLKPSNNVLFSVGSTIYHEKRDLLRSWHLPSVFHLRGKQ |
| | | | NYTCRCETIVSCDGYVVKRIAISPGLYGKPSGYAATMHR |
| | | | EGFLCCKVTDTLNGERVSFPVCTYVPATLCDQMTGILAT |
| | | | DVSADDAQKLLVGLNQRIVVNGRTQRNTNTMKNYLLPV |
| | | | VAQAFARWAKEYKEDQEDERPLGLRDRQLVMGCCWAF |
| | | | RRHKITSIYKRPDTQTIIKVNSDFHSFVLPRIGSNTLEIGLR |
| | | | TRIRKMLEEHKEPSPLITAEDVQEAKCAADEAKEVREAE |
| | | | ELRAALPPLAADVEEPTLEADVDLMLQEAGAGSVETPRG |
| | | | LIKVTSYDGEDKIGSYAVLSPQAVLKSEKLSCIHPLAEQVI |
| | | | VITHSGRKGRYAVEPYHGKVVVPEGHAIPVQDFQALSES |
| | | | ATIVYNEREFVNRYLHHIATHGGALNTDEEYYKTVKPSE |
| | | | HDGEYLYDIDRKQCVKKELVTGLGLTGELVDPPFHEFAY |
| | | | ESLRTRPAAPYQVPTIGVYGVPGSGKSGIIKSAVTKKDLV |
| | | | VSAKKENCAEIIRDVKKMKGLDVNARTVDSVLLNGCKH |
| | | | PVETLYIDEAFACHAGTLRALIAIIRPKKAVLCGDPKQCG |
| | | | FFNMMCLKVHFNHEICTQVFHKSISRRCTKSVTSVVSTLF |
| | | | YDKKMRTTNPKETKIVIDTTGSTKPKQDDLILTCFRGWV |
| | | | KQLQIDYKGNEIMTAAASQGLTRKGVYAVRYKVNENPL |
| | | | YAPTSEHVNVLLTRTEDRIVWKTLAGDPWIKTLTAKYPG |
| | | | NFTATIEEWQAEHDAIMRHILERPDPTDVFQNKANVCWA |
| | | | KALVPVLKTAGIDMTTEQWNTVDYFETDKAHSAEIVLN |
| | | | QLCVRFFGLDLDSGLFSAPTVPLSIRNNHWDNSPSPNMY |
| | | | GLNKEVVRQLSRRYPQLPRAVATGRVYDMNTGTLRNYD |
| | | | PRINLVPVNRRLPHALVLHHNEHPQSDFSSFVSKLKGRTV |
| | | | LVVGEKLSVPGKMVDWLSDRPEATFRARLDLGIPGDVP |
| | | | KYDIIFVNVRTPYKYHHYQQCEDHAIKLSMLTKKACLHL |
| | | | NPGGTCVSIGYGYADRASESIIGAIARLFKFSRVCKPKSSL |
| | | | EETEVLFVFIGYDRKARTHNPYKLSSTLTNIYTGSRLHEA |
| | | | GCAPSYHVVRGDIATATEGVIINAANSKGQPGGGVCGAL |
| | | | YKKFPESFDLQPIEVGKARLVKGAAKHIIHAVGPNFNKVS |
| | | | EVEGDKQLAEAYESIAKIVNDNNYKSVAIPLLSTGIFSGN |
| | | | KDRLTQSLNHLLTALDTTDADVAIYCRDKKWEMTLKEA |
| | | | VARREAVEEICISDDSSVTEPDAELVRVHPKSSLAGRKGY |
| | | | STSDGKTFSYLEGTKFHQAAKDIAEINAMWPVATEANEQ |
| | | | VCMYILGESMSSIRSKCPVEESEASTPPSTLPCLCIHAMTP |
| | | | ERVQRLKASRPEQITVCSSFPLPKYRITGVQKIQCSQPILFS |
| | | | PKVPAYIHPRKYLVETPPVDETPEPSAENQSTEGTPEQPPL |
| | | | ITEDETRTRTPEPIIIEEEEEDSISLLSDGPTHQVLQVEADIH |
| | | | GPPSVSSSSWSIPHASDFDVDSLSILDTLEGASVTSGATSA |
| | | | ETNSYFAKSMEFLARPVPAPRTVFRNPPHPAPRTRTPSLA |
| | | | PSRACSRTSLVSTPPGVNRVITREELEALTPSRTPSRSVSR |
| | | | TSLVSNPPGVNRVITREEFEAFVAQQQRRFDAGAYIFSSD |
| | | | TGQGHLQQKSVRQTVLSEVVLERTELEISYAPRLDQEKE |
| | | | ELLRKKLQLNPTPANRSRYQSRKVENMKAITARRILQGL |
| | | | GHYLKAEGKVECYRTLHPVPLYSSSVNRAFSSPKVAVEA |
| | | | CNAMLKENFPTVASYCIIPEYDAYLDMVDGASCCLDTAS |
| | | | FCPAKLRSFPKKHSYLEPTIRSAVPSAIQNTLQNVLAAAT |
| | | | KRNCNVTQMRELPVLDSAAFNVECFKKYACNNEYWETF |
| | | | KENPIRLTEENVVNYITKLKGPKAAALFAKTHNLNMLQD |
| | | | IPMDRFVMDLKRDVKVTPGTKHTEERPKVQVIQAADPL |
| | | | ATAYLCGIHRELVRRLNAVLLPNIHTLFDMSAEDFDAIIA |
| | | | EHFQPGDCVLETDIASFDKSEDDAMALTALMILEDLGVD |
| | | | AELLTLIEAAFGEISSIHLPTKTKFKFGAMMKSGMFLTLF |
| | | | VNTVINIVIASRVLRERLTGSPCAAFIGDDNIVKGVKSDK |
| | | | LMADRCATWLNMEVKIIDAVVGEKAPYFCGGFILCDSVT |
| | | | GTACRVADPLKRLFKLGKPLAADDEHDDDRRRALHEES |
| | | | TRWNRVGILSELCKAVESRYETVGTSIIVMAMTTLASSV |
| | | | KSFSYLRGAPITLYG* |
| STARR ™ | intergenic | nucleotide | CCTGAATGGACTACGACATAGTCTAGTCCGCCAAGGC |
| (SEQ ID | region | | CGCCACC |
| NO: 52) | | | |
| STARR ™ | transgene | nucleotide | n/a (depends on gene of our interest) |
| | ORF | | |
| STARR ™ | 3' UTR | nucleotide | ACTCGAGTATGTTACGTGCAAAGGTGATTGTCACCCCC |
| (SEQ ID | | | CGAAAGACCATATTGTGACACACCCTCAGTATCACGC |
| NO: 53) | | | CCAAACATTTACAGCCGCGGTGTCAAAAACCGCGTGG |
| | | | ACGTGGTTAACATCCCTGCTGGGAGGATCAGCCGTAA |
| | | | TTATTATAATTGGCTTGGTGCTGGCTACTATTGTGGCC |
| | | | ATGTACGTGCTGACCAACCAGAAACATAATTGAATAC |
| | | | AGCAGCAATTGGCAAGCTGCTTACATAGAACTCGCGG |
| | | | CGATTGGCATGCCGCCTTAAAATTTTTATTTTATTTTTT |
| | | | CTTTTCTTTTCCGAATCGGATTTTGTTTTTAATATTTCA |
| | | | AAAAAAAAAAAAAAAAAAAAAATCTAGAAAAAAA |
| | | | AAAAAAAAAAAAAAAAAAAAAAAAAAAAAAAAAAA |
| | | | AAAAAAAAAAAAAAAAAAAAAAAAAAAAAAAAAAA |
| | | | AAAAAAAAAAAAAAAAAAAAAAA |
| Comparitive | 5' UTR | nucleotide | unknown |
| Original | non- | nucleotide | ATGCCCGAGAAGGTGCACGTGGACATCGAGGAGGACA |
| (SEQ ID | structural | | GCCCCTTCCTGAGGGCCCTGCAGAGGAGCTTCCCACA |
| NO: 54) | gene ORF | | GTTCGAAGTGGAGGCCAAGCAGGTGACCGACAACGAC |
| | | | CACGCCAACGCCAGGGCCTTCAGCCACCTGGCCAGCA |
| | | | AGCTGATCGAGACCGAGGTGGACCCCAGCGACACCAT |
| | | | CCTGGACATCGGCAGCGCCCCAGCCAGGAGAATGTAC |
| | | | AGCAAGCACAAGTACCACTGCATCTGCCCCATGAGGT |
| | | | GCGCCGAGGACCCCGACAGGCTGTACAAGTACGCCAC |
| | | | CAAACTGAAGAAGAACTGCAAGGAGATCACCGACAA |
| | | | GGAGCTGGACAAGAAAATGAAGGAGCTGGCCGCCGTG |
| | | | ATGAGCGACCCCGACCTGGAGACCGAGACAATGTGCC |
| | | | TGCACGACGACGAGAGCTGCAGGTACGAGGGCCAGGT |
| | | | GGCCGTCTACCAGGACGTGTACGCCGTCGACGGCCCC |
| | | | ACCAGCCTGTACCACCAGGCCAACAAGGGCGTGAGGG |
| | | | TGGCCTACTGGATCGGCTTCGACACCACACCCTTCATG |
| | | | TTCAAGAACCTGGCCGGCGCCTACCCCAGCTACAGCA |
| | | | CCAACTGGGCCGACGAGACCGTGCTGACCGCCAGGAA |
| | | | CATCGGCCTGTGCAGCAGCGACGTGATGGAGAGGAGC |
| | | | CGGAGAGGCATGAGCATCCTGAGGAAGAAATACCTGA |
| | | | AGCCCAGCAACAACGTGCTGTTCAGCGTGGGCAGCAC |
| | | | CATCTACCACGAGAAGAGGGACCTGCTCAGGAGCTGG |
| | | | CACCTGCCCAGCGTGTTCCACCTGAGGGGCAAGCAGA |
| | | | ACTACACCTGCAGGTGCGAGACCATCGTGAGCTGCGA |
| | | | CGGCTACGTGGTGAAGAGGATCGCCATCAGCCCCGGC |
| | | | CTGTACGGCAAGCCCAGCGGCTACGCCGCTACAATGC |
| | | | ACAGGGAGGGCTTCCTGTGCTGCAAGGTGACCGACAC |
| | | | CCTGAACGGCGAGAGGGTGAGCTTCCCCGTGTGCACC |
| | | | TACGTGCCCGCCACCCTGTGCGACCAGATGACCGGCA |
| | | | TCCTGGCCACCGACGTGAGCGCCGACGACGCCCAGAA |
| | | | GCTGCTCGTGGGCCTGAACCAGAGGATCGTGGTCAAC |
| | | | GGCAGGACCCAGAGGAACACCAACACAATGAAGAAC |
| | | | TACCTGCTGCCCGTGGTGGCCCAGGCTTTCGCCAGGTG |
| | | | GGCCAAGGAGTACAAGGAGGACCAGGAAGACGAGAG |
| | | | GCCCCTGGGCCTGAGGGACAGGCAGCTGGTGATGGGC |
| | | | TGCTGCTGGGCCTTCAGGCGGCACAAGATCACCAGCA |
| | | | TCTACAAGAGGCCCGACACCCAGACCATCATCAAGGT |
| | | | GAACAGCGACTTCCACAGCTTCGTGCTGCCCAGGATC |
| | | | GGCAGCAACACCCTGGAGATCGGCCTGAGGACCCGGA |
| | | | TCAGGAAGATGCTGGAGGAACACAAGGAGCCCAGCCC |
| | | | ACTGATCACCGCCGAGGACGTGCAGGAGGCCAAGTGC |
| | | | GCTGCCGACGAGGCCAAGGAGGTGAGGGAGGCCGAG |
| | | | GAACTGAGGGCCGCCCTGCCACCCCTGGCTGCCGACG |
| | | | TGGAGGAACCCACCCTGGAAGCCGACGTGGACCTGAT |
| | | | GCTGCAGGAGGCCGGCGCCGGAAGCGTGGAGACACCC |
| | | | AGGGGCCTGATCAAGGTGACCAGCTACGACGGCGAGG |
| | | | ACAAGATCGGCAGCTACGCCGTGCTGAGCCCACAGGC |
| | | | CGTGCTGAAGTCCGAGAAGCTGAGCTGCATCCACCCA |
| | | | CTGGCCGAGCAGGTGATCGTGATCACCCACAGCGGCA |
| | | | GGAAGGGCAGGTACGCCGTGGAGCCCTACCACGGCAA |
| | | | GGTGGTCGTGCCCGAGGGCCACGCCATCCCCGTGCAG |
| | | | GACTTCCAGGCCCTGAGCGAGAGCGCCACCATCGTGT |
| | | | ACAACGAGAGGGAGTTCGTGAACAGGTACCTGCACCA |
| | | | TATCGCCACCCACGGCGGAGCCCTGAACACCGACGAG |
| | | | GAATACTACAAGACCGTGAAGCCCAGCGAGCACGACG |
| | | | GCGAGTACCTGTACGACATCGACAGGAAGCAGTGCGT |
| | | | GAAGAAAGAGCTGGTGACCGGCCTGGGACTGACCGGC |
| | | | GAGCTGGTGGACCCACCCTTCCACGAGTTCGCCTACGA |
| | | | GAGCCTGAGGACCAGACCCGCCGCTCCCTACCAGGTG |
| | | | CCCACCATCGGCGTGTACGGCGTGCCCGGCAGCGGAA |
| | | | AGAGCGGCATCATCAAGAGCGCCGTGACCAAGAAAGA |
| | | | CCTGGTGGTCAGCGCCAAGAAAGAGAACTGCGCCGAG |
| | | | ATCATCAGGGACGTGAAGAAGATGAAAGGCCTGGACG |
| | | | TGAACGCGCGCACCGTGGACAGCGTGCTGCTGAACGG |
| | | | CTGCAAGCACCCCGTGGAGACCCTGTACATCGACGAG |
| | | | GCCTTCGCTTGCCACGCCGGCACCCTGAGGGCCCTGAT |
| | | | CGCCATCATCAGGCCCAAGAAAGCCGTGCTGTGCGGC |
| | | | GACCCCAAGCAGTGCGGCTTCTTCAACATGATGTGCCT |
| | | | GAAGGTGCACTTCAACCACGAGATCTGCACCCAGGTG |
| | | | TTCCACAAGAGCATCAGCAGGCGGTGCACCAAGAGCG |
| | | | TGACCAGCGTCGTGAGCACCCTGTTCTACGACAAGAA |
| | | | AATGAGGACCACCAACCCCAAGGAGACCAAAATCGTG |
| | | | ATCGACACCACAGGCAGCACCAAGCCCAAGCAGGACG |
| | | | ACCTGATCCTGACCTGCTTCAGGGGCTGGGTGAAGCA |
| | | | GCTGCAGATCGACTACAAGGGCAACGAGATCATGACC |
| | | | GCCGCTGCCAGCCAGGGCCTGACCAGGAAGGGCGTGT |
| | | | ACGCCGTGAGGTACAAGGTGAACGAGAACCCACTGTA |
| | | | CGCTCCCACCAGCGAGCACGTGAACGTGCTGCTGACC |
| | | | AGGACCGAGGACAGGATCGTGTGGAAGACCCTGGCCG |
| | | | GCGACCCCTGGATCAAGACCCTGACCGCCAAGTACCC |
| | | | CGGCAACTTCACCGCCACCATCGAAGAGTGGCAGGCC |
| | | | GAGCACGACGCCATCATGAGGCACATCCTGGAGAGGC |
| | | | CCGACCCCACCGACGTGTTCCAGAACAAGGCCAACGT |
| | | | GTGCTGGGCCAAGGCCCTGGTGCCCGTGCTGAAGACC |
| | | | GCCGGCATCGACATGACCACAGAGCAGTGGAACACCG |
| | | | TGGACTACTTCGAGACCGACAAGGCCCACAGCGCCGA |
| | | | GATCGTGCTGAACCAGCTGTGCGTGAGGTTCTTCGGCC |
| | | | TGGACCTGGACAGCGGCCTGTTCAGCGCCCCCACCGT |
| | | | GCCACTGAGCATCAGGAACAACCACTGGGACAACAGC |
| | | | CCCAGCCCAAACATGTACGGCCTGAACAAGGAGGTGG |
| | | | TCAGGCAGCTGAGCAGGCGGTACCCACAGCTGCCCAG |
| | | | GGCCGTGGCCACCGGCAGGGTGTACGACATGAACACC |
| | | | GGCACCCTGAGGAACTACGACCCCAGGATCAACCTGG |
| | | | TGCCCGTGAACAGGCGGCTGCCCCACGCCCTGGTGCT |
| | | | GCACCACAACGAGCACCCACAGAGCGACTTCAGCTCC |
| | | | TTCGTGAGCAAGCTGAAAGGCAGGACCGTGCTGGTCG |
| | | | TGGGCGAGAAGCTGAGCGTGCCCGGCAAGATGGTGGA |
| | | | CTGGCTGAGCGACAGGCCCGAGGCCACCTTCCGGGCC |
| | | | AGGCTGGACCTCGGCATCCCCGGCGACGTGCCCAAGT |
| | | | ACGACATCATCTTCGTGAACGTCAGGACCCCATACAA |
| | | | GTACCACCATTACCAGCAGTGCGAGGACCACGCCATC |
| | | | AAGCTGAGCATGCTGACCAAGAAGGCCTGCCTGCACC |
| | | | TGAACCCCGGAGGCACCTGCGTGAGCATCGGCTACGG |
| | | | CTACGCCGACAGGGCCAGCGAGAGCATCATTGGCGCC |
| | | | ATCGCCAGGCTGTTCAAGTTCAGCAGGGTGTGCAAAC |
| | | | CCAAGAGCAGCCTGGAGGAAACCGAGGTGCTGTTCGT |
| | | | GTTCATCGGCTACGACCGGAAGGCCAGGACCCACAAC |
| | | | CCCTACAAGCTGAGCAGCACCCTGACAAACATCTACA |
| | | | CCGGCAGCAGGCTGCACGAGGCCGGCTGCGCCCCCAG |
| | | | CTACCACGTGGTCAGGGGCGATATCGCCACCGCCACC |
| | | | GAGGGCGTGATCATCAACGCTGCCAACAGCAAGGGCC |
| | | | AGCCCGGAGGCGGAGTGTGCGGCGCCCTGTACAAGAA |
| | | | GTTCCCCGAGAGCTTCGACCTGCAGCCCATCGAGGTG |
| | | | GGCAAGGCCAGGCTGGTGAAGGGCGCCGCTAAGCACA |
| | | | TCATCCACGCCGTGGGCCCCAACTTCAACAAGGTGAG |
| | | | CGAGGTGGAAGGCGACAAGCAGCTGGCCGAAGCCTAC |
| | | | GAGAGCATCGCCAAGATCGTGAACGACAATAACTACA |
| | | | AGAGCGTGGCCATCCCACTGCTCAGCACCGGCATCTTC |
| | | | AGCGGCAACAAGGACAGGCTGACCCAGAGCCTGAACC |
| | | | ACCTGCTCACCGCCCTGGACACCACCGATGCCGACGT |
| | | | GGCCATCTACTGCAGGGACAAGAAGTGGGAGATGACC |
| | | | CTGAAGGAGGCCGTGGCCAGGCGGGAGGCCGTGGAA |
| | | | GAGATCTGCATCAGCGACGACTCCAGCGTGACCGAGC |
| | | | CCGACGCCGAGCTGGTGAGGGTGCACCCCAAGAGCTC |
| | | | CCTGGCCGGCAGGAAGGGCTACAGCACCAGCGACGGC |
| | | | AAGACCTTCAGCTACCTGGAGGGCACCAAGTTCCACC |
| | | | AGGCCGCTAAGGACATCGCCGAGATCAACGCTATGTG |
| | | | GCCCGTGGCCACCGAGGCCAACGAGCAGGTGTGCATG |
| | | | TACATCCTGGGCGAGAGCATGTCCAGCATCAGGAGCA |
| | | | AGTGCCCCGTGGAGGAAAGCGAGGCCAGCACACCACC |
| | | | CAGCACCCTGCCCTGCCTGTGCATCCACGCTATGACAC |
| | | | CCGAGAGGGTGCAGCGGCTGAAGGCCAGCAGGCCCGA |
| | | | GCAGATCACCGTGTGCAGCTCCTTCCCACTGCCCAAGT |
| | | | ACAGGATCACCGGCGTGCAGAAGATCCAGTGCAGCCA |
| | | | GCCCATCCTGTTCAGCCCAAAGGTGCCCGCCTACATCC |
| | | | ACCCCAGGAAGTACCTGGTGGAGACCCCACCCGTGGA |
| | | | CGAGACACCCGAGCCAAGCGCCGAGAACCAGAGCACC |
| | | | GAGGGCACACCCGAGCAGCCACCCCTGATCACCGAGG |
| | | | ACGAGACAAGGACCCGGACCCCAGAGCCCATCATTAT |
| | | | CGAGGAAGAGGAAGAGGACAGCATCAGCCTGCTGAG |
| | | | CGACGGCCCCACCCACCAGGTGCTGCAGGTGGAGGCC |
| | | | GACATCCACGGCCCACCCAGCGTGTCCAGCTCCAGCT |
| | | | GGAGCATCCCACACGCCAGCGACTTCGACGTGGACAG |
| | | | CCTGAGCATCCTGGACACCCTGGAGGGCGCCAGCGTG |
| | | | ACCTCCGGCGCCACCAGCGCCGAGACCAACAGCTACT |
| | | | TCGCCAAGAGCATGGAGTTCCTGGCCAGGCCCGTGCC |
| | | | AGCTCCCAGGACCGTGTTCAGGAACCCACCCCACCCA |
| | | | GCTCCCAGGACCAGGACCCCAAGCCTGGCTCCCAGCA |
| | | | GGGCCTGCAGCAGGACCAGCCTGGTGAGCACCCCACC |
| | | | CGGCGTGAACAGGGTGATCACCAGGGAGGAACTGGAG |
| | | | GCCCTGACACCCAGCAGGACCCCCAGCAGGTCCGTGA |
| | | | GCAGGACTAGTCTGGTGTCCAACCCACCCGGCGTGAA |
| | | | CAGGGTGATCACCAGGGAGGAATTCGAGGCCTTCGTG |
| | | | GCCCAGCAACAGAGACGGTTCGACGCCGGCGCCTACA |
| | | | TCTTCAGCAGCGACACCGGCCAGGGACACCTGCAGCA |
| | | | AAAGAGCGTGAGGCAGACCGTGCTGAGCGAGGTGGTG |
| | | | CTGGAGAGGACCGAGCTGGAAATCAGCTACGCCCCCA |
| | | | GGCTGGACCAGGAGAAGGAGGAACTGCTCAGGAAGA |
| | | | AACTGCAGCTGAACCCCACCCCAGCCAACAGGAGCAG |
| | | | GTACCAGAGCAGGAAGGTGGAGAACATGAAGGCCATC |
| | | | ACCGCCAGGCGGATCCTGCAGGGCCTGGGACACTACC |
| | | | TGAAGGCCGAGGGCAAGGTGGAGTGCTACAGGACCCT |
| | | | GCACCCCGTGCCACTGTACAGCTCCAGCGTGAACAGG |
| | | | GCCTTCTCCAGCCCCAAGGTGGCCGTGGAGGCCTGCA |
| | | | ACGCTATGCTGAAGGAGAACTTCCCCACCGTGGCCAG |
| | | | CTACTGCATCATCCCCGAGTACGACGCCTACCTGGACA |
| | | | TGGTGGACGGCGCCAGCTGCTGCCTGGACACCGCCAG |
| | | | CTTCTGCCCCGCCAAGCTGAGGAGCTTCCCCAAGAAA |
| | | | CACAGCTACCTGGAGCCCACCATCAGGAGCGCCGTGC |
| | | | CCAGCGCCATCCAGAACACCCTGCAGAACGTGCTGGC |
| | | | CGCTGCCACCAAGAGGAACTGCAACGTGACCCAGATG |
| | | | AGGGAGCTGCCCGTGCTGGACAGCGCTGCCTTCAACG |
| | | | TGGAGTGCTTCAAGAAATACGCCTGCAACAACGAGTA |
| | | | CTGGGAGACCTTCAAGGAGAACCCCATCAGGCTGACC |
| | | | GAAGAGAACGTGGTGAACTACATCACCAAGCTGAAGG |
| | | | GCCCCAAGGCCGCTGCCCTGTTCGCTAAGACCCACAA |
| | | | CCTGAACATGCTGCAGGACATCCCAATGGACAGGTTC |
| | | | GTGATGGACCTGAAGAGGGACGTGAAGGTGACACCCG |
| | | | GCACCAAGCACACCGAGGAGAGGCCCAAGGTGCAGGT |
| | | | GATCCAGGCCGCTGACCCACTGGCCACCGCCTACCTGT |
| | | | GCGGCATCCACAGGGAGCTGGTGAGGCGGCTGAACGC |
| | | | CGTGCTGCTGCCCAACATCCACACCCTGTTCGACATGA |
| | | | GCGCCGAGGACTTCGACGCCATCATCGCCGAGCACTT |
| | | | CCAGCCCGGCGACTGCGTGCTGGAGACCGACATCGCC |
| | | | AGCTTCGACAAGAGCGAGGATGACGCTATGGCCCTGA |
| | | | CCGCTCTGATGATCCTGGAGGACCTGGGCGTGGACGC |
| | | | CGAGCTGCTCACCCTGATCGAGGCTGCCTTCGGCGAG |
| | | | ATCAGCTCCATCCACCTGCCCACCAAGACCAAGTTCAA |
| | | | GTTCGGCGCTATGATGAAAAGCGGAATGTTCCTGACC |
| | | | CTGTTCGTGAACACCGTGATCAACATTGTGATCGCCAG |
| | | | CAGGGTGCTGCGGGAGAGGCTGACCGGCAGCCCCTGC |
| | | | GCTGCCTTCATCGGCGACGACAACATCGTGAAGGGCG |
| | | | TGAAAAGCGACAAGCTGATGGCCGACAGGTGCGCCAC |
| | | | CTGGCTGAACATGGAGGTGAAGATCATCGACGCCGTG |
| | | | GTGGGCGAGAAGGCCCCCTACTTCTGCGGCGGATTCA |
| | | | TCCTGTGCGACAGCGTGACCGGCACCGCCTGCAGGGT |
| | | | GGCCGACCCCCTGAAGAGGCTGTTCAAGCTGGGCAAG |
| | | | CCACTGGCCGCTGACGATGAGCACGACGATGACAGGC |
| | | | GGAGGGCCCTGCACGAGGAAAGCACCAGGTGGAACA |
| | | | GGGTGGGCATCCTGAGCGAGCTGTGCAAGGCCGTGGA |
| | | | GAGCAGGTACGAGACCGTGGGCACCAGCATCATCGTG |
| | | | ATGGCTATGACCACACTGGCCAGCTCCGTCAAGAGCTT |
| | | | CTCCTACCTGAGGGGGGCCCCTATAACTCTCTACGGCT |
| | | | AA |
| Comparitive | non- | amino acid | MPEKVHVDIEEDSPFLRALQRSFPQFEVEAKQVTDNDHA |
| (SEQ ID | structural | | NARAFSHLASKLIETEVDPSDTILDIGSAPARRMYSKHKY |
| NO: 55) | gene ORF | | HCICPMRCAEDPDRLYKYATKLKKNCKEITDKELDKKM |
| | | | KELAAVMSDPDLETETMCLHDDESCRYEGQVAVYQDV |
| | | | YAVDGPTSLYHQANKGVRVAYWIGFDTTPFMFKNLAGA |
| | | | YPSYSTNWADETVLTARNIGLCSSDVMERSRRGMSILRK |
| | | | KYLKPSNNVLFSVGSTIYHEKRDLLRSWHLPSVFHLRGK |
| | | | QNYTCRCETIVSCDGYVVKRIAISPGLYGKPSGYAATMH |
| | | | REGFLCCKVTDTLNGERVSFPVCTYVPATLCDQMTGILA |
| | | | TDVSADDAQKLLVGLNQRIVVNGRTQRNTNTMKNYLLP |
| | | | VVAQAFARWAKEYKEDQEDERPLGLRDRQLVMGCCWA |
| | | | FRRHKITSIYKRPDTQTIIKVNSDFHSFVLPRIGSNTLEIGL |
| | | | RTRIRKMLEEHKEPSPLITAEDVQEAKCAADEAKEVREA |
| | | | EELRAALPPLAADVEEPTLEADVDLMLQEAGAGSVETPR |
| | | | GLIKVTSYDGEDKIGSYAVLSPQAVLKSEKLSCIHPLAEQ |
| | | | VIVITHSGRKGRYAVEPYHGKVVVPEGHAIPVQDFQALS |
| | | | ESATIVYNEREFVNRYLHHIATHGGALNTDEEYYKTVKP |
| | | | SEHDGEYLYDIDRKQCVKKELVTGLGLTGELVDPPFHEF |
| | | | AYESLRTRPAAPYQVPTIGVYGVPGSGKSGIIKSAVTKKD |
| | | | LVVSAKKENCAEIIRDVKKMKGLDVNARTVDSVLLNGC |
| | | | KHPVETLYIDEAFACHAGTLRALIAIIRPKKAVLCGDPKQ |
| | | | CGFFNMMCLKVHFNHEICTQVFHKSISRRCTKSVTSVVS |
| | | | TLFYDKKMRTTNPKETKIVIDTTGSTKPKQDDLILTCFRG |
| | | | WVKQLQIDYKGNEIMTAAASQGLTRKGVYAVRYKVNE |
| | | | NPLYAPTSEHVNVLLTRTEDRIVWKTLAGDPWIKTLTAK |
| | | | YPGNFTATIEEWQAEHDAIMRHILERPDPTDVFQNKANV |
| | | | CWAKALVPVLKTAGIDMTTEQWNTVDYFETDKAHSAEI |
| | | | VLNQLCVRFFGLDLDSGLFSAPTVPLSIRNNHWDNSPSPN |
| | | | MYGLNKEVVRQLSRRYPQLPRAVATGRVYDMNTGTLR |
| | | | NYDPRINLVPVNRRLPHALVLHHNEHPQSDFSSFVSKLK |
| | | | GRTVLVVGEKLSVPGKMVDWLSDRPEATFRARLDLGIP |
| | | | GDVPKYDIIFVNVRTPYKYHHYQQCEDHAIKLSMLTKKA |
| | | | CLHLNPGGTCVSIGYGYADRASESIIGAIARLFKFSRVCKP |
| | | | KSSLEETEVLFVFIGYDRKARTHNPYKLSSTLTNIYTGSRL |
| | | | HEAGCAPSYHVVRGDIATATEGVIINAANSKGQPGGGVC |
| | | | GALYKKFPESFDLQPIEVGKARLVKGAAKHIIHAVGPNF |
| | | | NKVSEVEGDKQLAEAYESIAKIVNDNNYKSVAIPLLSTGI |
| | | | FSGNKDRLTQSLNHLLTALDTTDADVAIYCRDKKWEMT |
| | | | LKEAVARREAVEEICISDDSSVTEPDAELVRVHPKSSLAG |
| | | | RKGYSTSDGKTFSYLEGTKFHQAAKDIAEINAMWPVATE |
| | | | ANEQVCMYILGESMSSIRSKCPVEESEASTPPSTLPCLCIH |
| | | | AMTPERVQRLKASRPEQITVCSSFPLPKYRITGVQKIQCS |
| | | | QPILFSPKVPAYIHPRKYLVETPPVDETPEPSAENQSTEGT |
| | | | PEQPPLITEDETRTRTPEPINIEEEEEDSISLLSDGPTHQVLQ |
| | | | VEADIHGPPSVSSSSWSIPHASDFDVDSLSILDTLEGASVT |
| | | | SGATSAETNSYFAKSMEFLARPVPAPRTVFRNPPHPAPRT |
| | | | RTPSLAPSRACSRTSLVSTPPGVNRVITREELEALTPSRTP |
| | | | SRSVSRTSLVSNPPGVNRVITREEFEAFVAQQQRRFDAGA |
| | | | YIFSSDTGQGHLQQKSVRQTVLSEVVLERTELEISYAPRL |
| | | | DQEKEELLRKKLQLNPTPANRSRYQSRKVENMKAITARR |
| | | | ILQGLGHYLKAEGKVECYRTLHPVPLYSSSVNRAFSSPK |
| | | | VAVEACNAMLKENFPTVASYCIIPEYDAYLDMVDGASC |
| | | | CLDTASFCPAKLRSFPKKHSYLEPTIRSAVPSAIQNTLQNV |
| | | | LAAATKRNCNVTQMRELPVLDSAAFNVECFKKYACNNE |
| | | | YWETFKENPIRLTEENVVNYITKLKGPKAAALFAKTHNL |
| | | | NMLQDIPMDRFVMDLKRDVKVTPGTKHTEERPKVQVIQ |
| | | | AADPLATAYLCGIHRELVRRLNAVLLPNIHTLFDMSAED |
| | | | FDAIIAEHFQPGDCVLETDIASFDKSEDDAMALTALMILE |
| | | | DLGVDAELLTLIEAAFGEISSIHLPTKTKFKFGAMMKSGM |
| | | | FLTLFVNTVINIVIASRVLRERLTGSPCAAFIGDDNIVKGV |
| | | | KSDKLMADRCATWLNMEVKIIDAVVGEKAPYFCGGFIL |
| | | | CDSVTGTACRVADPLKRLFKLGKPLAADDEHDDDRRRA |
| | | | LHEESTRWNRVGILSELCKAVESRYETVGTSIIVMAMTTL |
| | | | ASSVKSFSYLRGAPITLYG* |
| Comparitive | intergenic | nucleotide | unknown |
| | region | | |
| Comparitive | 3' UTR | nucleotide | unknown |

TABLE 5
ORF of Peptide of Interest for
Self-Replicating RNAs of the Disclosure

| ORF | Sequence | |
|---|---|---|
| Identity | Type | Sequence |
| 2019-nCoV | nucleotide | ATGTTTGTTTTTCTTGTTTTATTGCCACTAGTCTCTAGTCAGTGTGT |
| Spike gene | | TAATCTTACAACCAGAACTCAATTACCCCCTGCATACACTAATTC |
| (SEQ ID | | TTTCACACGTGGTGTTTATTACCCTGACAAAGTTTTCAGATCCTCA |
| NO: 117) | | GTTTTACATTCAACTCAGGACTTGTTCTTACCTTTCTTTTCCAATGT |
| | | TACTTGGTTCCATGCTATACATGTCTCTGGGACCAATGGTACTAA |
| | | GAGGTTTGATAACCCTGTCCTACCATTTAATGATGGTGTTTATTTT |
| | | GCTTCCACTGAGAAGTCTAACATAATAAGAGGCTGGATTTTTGGT |
| | | ACTACTTTAGATTCGAAGACCCAGTCCCTACTTATTGTTAATAAC |
| | | GCTACTAATGTTGTTATTAAAGTCTGTGAATTTCAATTTTGTAATG |
| | | ATCCATTTTTGGGTGTTTATTACCACAAAAACAACAAAAGTTGGA |
| | | TGGAAAGTGAGTTCAGAGTTTATTCTAGTGCGAATAATTGCACTT |
| | | TTGAATATGTCTCTCAGCCTTTTCTTATGGACCTTGAAGGAAAAC |
| | | AGGGTAATTTCAAAAATCTTAGGGAATTTGTGTTTAAGAATATTG |
| | | ATGGTTATTTTAAAATATATTCTAAGCACACGCCTATTAATTTAGT |
| | | GCGTGATCTCCCTCAGGGTTTTTCGGCTTTAGAACCATTGGTAGAT |
| | | TTGCCAATAGGTATTAACATCACTAGGTTTCAAACTTTACTTGCTT |
| | | TACATAGAAGTTATTTGACTCCTGGTGATTCTTCTTCAGGTTGGAC |
| | | AGCTGGTGCTGCAGCTTATTATGTGGGTTATCTTCAACCTAGGAC |
| | | TTTTCTATTAAAATATAATGAAAATGGAACCATTACAGATGCTGT |
| | | AGACTGTGCACTTGACCCTCTCTCAGAAACAAAGTGTACGTTGAA |
| | | ATCCTTCACTGTAGAAAAAGGAATCTATCAAACTTCTAACTTTAG |
| | | AGTCCAACCAACAGAATCTATTGTTAGATTTCCTAATATTACAAA |
| | | CTTGTGCCCTTTTGGTGAAGTTTTTAACGCCACCAGATTTGCATCT |
| | | GTTTATGCTTGGAACAGGAAGAGAATCAGCAACTGTGTTGCTGAT |
| | | TATTCTGTCCTATATAATTCCGCATCATTTTCCACTTTTAAGTGTT |
| | | ATGGAGTGTCTCCTACTAAATTAAATGATCTCTGCTTTACTAATGT |
| | | CTATGCAGATTCATTTGTAATTAGAGGTGATGAAGTCAGACAAAT |
| | | CGCTCCAGGGCAAACTGGAAAGATTGCTGATTATAATTATAAATT |
| | | ACCAGATGATTTTACAGGCTGCGTTATAGCTTGGAATTCTAACAA |
| | | TCTTGATTCTAAGGTTGGTGGTAATTATAATTACCTGTATAGATTG |
| | | TTTAGGAAGTCTAATCTCAAACCTTTTGAGAGAGATATTTCAACT |
| | | GAAATCTATCAGGCCGGTAGCACACCTTGTAATGGTGTTGAAGGT |
| | | TTTAATTGTTACTTTCCTTTACAATCATATGGTTTCCAACCCACTA |
| | | ATGGTGTTGGTTACCAACCATACAGAGTAGTAGTACTTTCTTTTG |
| | | AACTTCTACATGCACCAGCAACTGTTTGTGGACCTAAAAAGTCTA |
| | | CTAATTTGGTTAAAAACAAATGTGTCAATTTCAACTTCAATGGTTT |
| | | AACAGGCACAGGTGTTCTTACTGAGTCTAACAAAAAGTTTCTGCC |
| | | TTTCCAACAATTTGGCAGAGACATTGCTGACACTACTGATGCTGT |
| | | CCGTGATCCACAGACACTTGAGATTCTTGACATTACACCATGTTC |
| | | TTTTGGTGGTGTCAGTGTTATAACACCAGGAACAAATACTTCTAA |
| | | CCAGGTTGCTGTTCTTTATCAGGATGTTAACTGCACAGAAGTCCC |
| | | TGTTGCTATTCATGCAGATCAACTTACTCCTACTTGGCGTGTTTAT |
| | | TCTACAGGTTCTAATGTTTTTCAAACACGTGCAGGCTGTTTAATAG |
| | | GGGCTGAACATGTCAACAACTCATATGAGTGTGACATACCCATTG |
| | | GTGCAGGTATATGCGCTAGTTATCAGACTCAGACTAATTCTCCTC |
| | | GGCGGGCACGTAGTGTAGCTAGTCAATCCATCATTGCCTACACTA |
| | | TGTCACTTGGTGCAGAAAATTCAGTTGCTTACTCTAATAACTCTAT |
| | | TGCCATACCCACAAATTTTACTATTAGTGTTACCACAGAAATTCT |
| | | ACCAGTGTCTATGACCAAGACATCAGTAGATTGTACAATGTACAT |
| | | TTGTGGTGATTCAACTGAATGCAGCAATCTTTTGTTGCAATATGG |
| | | CAGTTTTTGTACACAATTAAACCGTGCTTTAACTGGAATAGCTGTT |
| | | GAACAAGACAAAAACACCCAAGAAGTTTTTGCACAAGTCAAACA |
| | | AATTTACAAAACACCACCAATTAAAGATTTTGGTGGTTTTAATTTT |
| | | TCACAAATATTACCAGATCCATCAAAACCAAGCAAGAGGTCATTT |
| | | ATTGAAGATCTACTTTTCAACAAAGTGACACTTGCAGATGCTGGC |
| | | TTCATCAAACAATATGGTGATTGCCTTGGTGATATTGCTGCTAGA |
| | | GACCTCATTTGTGCACAAAAGTTTAACGGCCTTACTGTTTTGCCAC |
| | | CTTTGCTCACAGATGAAATGATTGCTCAATACACTTCTGCACTGTT |
| | | AGCGGGTACAATCACTTCTGGTTGGACCTTTGGTGCAGGTGCTGC |
| | | ATTACAAATACCATTTGCTATGCAAATGGCTTATAGGTTTAATGG |
| | | TATTGGAGTTACACAGAATGTTCTCTATGAGAACCAAAAATTGAT |
| | | TGCCAACCAATTTAATAGTGCTATTGGCAAAATTCAAGACTCACT |
| | | TTCTTCCACAGCAAGTGCACTTGGAAAACTTCAAGATGTGGTCAA |
| | | CCAAAATGCACAAGCTTTAAACACGCTTGTTAAACAACTTAGCTC |
| | | CAATTTTGGTGCAATTTCAAGTGTTTTAAATGATATCCTTTCACGT |
| | | CTTGACAAAGTTGAGGCTGAAGTGCAAATTGATAGGTTGATCACA |
| | | GGCAGACTTCAAAGTTTGCAGACATATGTGACTCAACAATTAATT |
| | | AGAGCTGCAGAAATCAGAGCTTCTGCTAATCTTGCTGCTACTAAA |
| | | ATGTCAGAGTGTGTACTTGGACAATCAAAAAGAGTTGATTTTTGT |
| | | GGAAAGGGCTATCATCTTATGTCCTTCCCTCAGTCAGCACCTCAT |
| | | GGTGTAGTCTTCTTGCATGTGACTTATGTCCCTGCACAAGAAAAG |
| | | AACTTCACAACTGCTCCTGCCATTTGTCATGATGGAAAAGCACAC |
| | | TTTCCTCGTGAAGGTGTCTTTGTTTCAAATGGCACACACTGGTTTG |
| | | TAACACAAAGGAATTITTATGAACCACAAATCATTACTACAGACA |
| | | ACACATTTGTGTCTGGTAACTGTGATGTTGTAATAGGAATTGTCA |
| | | ACAACACAGTTTATGATCCTTTGCAACCTGAATTAGACTCATTCA |
| | | AGGAGGAGTTAGATAAATATTTTAAGAATCATACATCACCAGATG |
| | | TTGATTTAGGTGACATCTCTGGCATTAATGCTTCAGTTGTAAACAT |
| | | TCAAAAAGAAATTGACCGCCTCAATGAGGTTGCCAAGAATTTAA |
| | | ATGAATCTCTCATCGATCTCCAAGAACTTGGAAAGTATGAGCAGT |
| | | ATATAAAATGGCCATGGTACATTTGGCTAGGTTTTATAGCTGGCT |
| | | TGATTGCCATAGTAATGGTGACAATTATGCTTTGCTGTATGACCA |
| | | GTTGCTGTAGTTGTCTCAAGGGCTGTTGTTCTTGTGGATCCTGCTG |
| | | CAAATTTGATGAAGACGACTCTGAGCCAGTGCTCAAAGGAGTCA |
| | | AATTACATTACACATAA |
| 2019-nCoV | amino acid | MFVFLVLLPLVSSQCVNLTTRTQLPPAYTNSFTRGVYYPDKVFRSSV |
| Spike gene | | LHSTQDLFLPFFSNVTWFHAIHVSGTNGTKRFDNPVLPFNDGVYFAS |
| (SEQ ID | | TEKSNIIRGWIFGTTLDSKTQSLLIVNNATNVVIKVCEFQFCNDPFLG |
| NO: 118) | | VYYHKNNKSWMESEFRVYSSANNCTFEYVSQPFLMDLEGKQGNFK |
| | | NLREFVFKNIDGYFKIYSKHTPINLVRDLPQGFSALEPLVDLPIGINITR |
| | | FQTLLALHRSYLTPGDSSSGWTAGAAAYYVGYLQPRTFLLKYNENG |
| | | TITDAVDCALDPLSETKCTLKSFTVEKGIYQTSNFRVQPTESIVRFPNI |
| | | TNLCPFGEVFNATRFASVYAWNRKRISNCVADYSVLYNSASFSTFKC |
| | | YGVSPTKLNDLCFTNVYADSFVIRGDEVRQIAPGQTGKIADYNYKLP |
| | | DDFTGCVIAWNSNNLDSKVGGNYNYLYRLFRKSNLKPFERDISTEIY |
| | | QAGSTPCNGVEGFNCYFPLQSYGFQPTNGVGYQPYRVVVLSFELLH |
| | | APATVCGPKKSTNLVKNKCVNFNFNGLTGTGVLTESNKKFLPFQQF |
| | | GRDIADTTDAVRDPQTLEILDITPCSFGGVSVITPGTNTSNQVAVLYQ |
| | | DVNCTEVPVAIHADQLTPTWRVYSTGSNVFQTRAGCLIGAEHVNNS |
| | | YECDIPIGAGICASYQTQTNSPRRARSVASQSIIAYTMSLGAENSVAY |
| | | SNNSIAIPTNFTISVTTEILPVSMTKTSVDCTMYICGDSTECSNLLLQY |
| | | GSFCTQLNRALTGIAVEQDKNTQEVFAQVKQIYKTPPIKDFGGFNFS |
| | | QILPDPSKPSKRSFIEDLLFNKVTLADAGFIKQYGDCLGDIAARDLICA |
| | | QKFNGLTVLPPLLTDEMIAQYTSALLAGTITSGWTFGAGAALQIPFA |
| | | MQMAYRFNGIGVTQNVLYENQKLIANQFNSAIGKIQDSLSSTASALG |
| | | KLQDVVNQNAQALNTLVKQLSSNFGAISSVLNDILSRLDKVEAEVQI |
| | | DRLITGRLQSLQTYVTQQLIRAAEIRASANLAATKMSECVLGQSKRV |
| | | DFCGKGYHLMSFPQSAPHGVVFLHVTYVPAQEKNFTTAPAICHDGK |
| | | AHFPREGVFVSNGTHWFVTQRNFYEPQUITTDNTFVSGNCDVVIGIVN |
| | | NTVYDPLQPELDSFKEELDKYFKNHTSPDVDLGDISGINASVVNIQKE |
| | | IDRLNEVAKNLNESLIDLQELGKYEQYIKWPWYIWLGFIAGLIAIVM |
| | | VTIMLCCMTSCCSCLKGCCSCGSCCKFDEDDSEPVLKGVKLHYT* |
| 2019-nCoV | nucleotide | ATGTTCGTCTTCCTGGTCCTGCTGCCTCTGGTCTCCTCACAGTGCG |
| Spike gene | | TCAATCTGACAACTCGGACTCAGCTGCCACCTGCTTATACTAATA |
| (SEQ ID | | GCTTCACCAGAGGCGTGTACTATCCTGACAAGGTGTTTAGAAGCT |
| NO: 119) | | CCGTGCTGCACTCTACACAGGATCTGTTTCTGCCATTCTTTAGCAA |
| | | CGTGACCTGGTTCCACGCCATCCACGTGAGCGGCACCAATGGCAC |
| | | AAAGCGGTTCGACAATCCCGTGCTGCCTTTTAACGATGGCGTGTA |
| | | CTTCGCCTCTACCGAGAAGAGCAACATCATCAGAGGCTGGATCTT |
| | | TGGCACCACACTGGACTCCAAGACACAGTCTCTGCTGATCGTGAA |
| | | CAATGCCACCAACGTGGTCATCAAGGTGTGCGAGTTCCAGTTTTG |
| | | TAATGATCCCTTCCTGGGCGTGTACTATCACAAGAACAATAAGAG |
| | | CTGGATGGAGTCCGAGTTTAGAGTGTATTCTAGCGCCAACAACTG |
| | | CACATTTGAGTACGTGAGCCAGCCTTTCCTGATGGACCTGGAGGG |
| | | CAAGCAGGGCAATTTCAAGAACCTGAGGGAGTTCGTGTTTAAGA |
| | | ATATCGACGGCTACTTCAAAATCTACTCTAAGCACACCCCCATCA |
| | | ACCTGGTGCGCGACCTGCCTCAGGGCTTCAGCGCCCTGGAGCCCC |
| | | TGGTGGATCTGCCTATCGGCATCAACATCACCCGGTTTCAGACAC |
| | | TGCTGGCCCTGCACAGAAGCTACCTGACACCCGGCGACTCCTCTA |
| | | GCGGATGGACCGCCGGCGCTGCCGCCTACTATGTGGGCTACCTCC |
| | | AGCCCCGGACCTTCCTGCTGAAGTACAACGAGAATGGCACCATCA |
| | | CAGACGCAGTGGATTGCGCCCTGGACCCCCTGAGCGAGACAAAG |
| | | TGTACACTGAAGTCCTTTACCGTGGAGAAGGGCATCTATCAGACA |
| | | TCCAATTTCAGGGTGCAGCCAACCGAGTCTATCGTGCGCTTTCCT |
| | | AATATCACAAACCTGTGCCCATTTGGCGAGGTGTTCAACGCAACC |
| | | CGCTTCGCCAGCGTGTACGCCTGGAATAGGAAGCGGATCAGCAA |
| | | CTGCGTGGCCGACTATAGCGTGCTGTACAACTCCGCCTCTTTCAG |
| | | CACCTTTAAGTGCTATGGCGTGTCCCCCACAAAGCTGAATGACCT |
| | | GTGCTTTACCAACGTCTACGCCGATTCTTTCGTGATCAGGGGCGA |
| | | CGAGGTGCGCCAGATCGCCCCCGGCCAGACAGGCAAGATCGCAG |
| | | ACTACAATTATAAGCTGCCAGACGATTTCACCGGCTGCGTGATCG |
| | | CCTGGAACAGCAACAATCTGGATTCCAAAGTGGGCGGCAACTAC |
| | | AATTATCTGTACCGGCTGTTTAGAAAGAGCAATCTGAAGCCCTTC |
| | | GAGAGGGACATCTCTACAGAAATCTACCAGGCCGGCAGCACCCC |
| | | TTGCAATGGCGTGGAGGGCTTTAACTGTTATTTCCCACTCCAGTCC |
| | | TACGGCTTCCAGCCCACAAACGGCGTGGGCTATCAGCCTTACCGC |
| | | GTGGTGGTGCTGAGCTTTGAGCTGCTGCACGCCCCAGCAACAGTG |
| | | TGCGGCCCCAAGAAGTCCACCAATCTGGTGAAGAACAAGTGCGT |
| | | GAACTTCAACTTCAACGGCCTGACCGGCACAGGCGTGCTGACCGA |
| | | GTCCAACAAGAAGTTCCTGCCATTTCAGCAGTTCGGCAGGGACAT |
| | | CGCAGATACCACAGACGCCGTGCGCGACCCACAGACCCTGGAGA |
| | | TCCTGGACATCACACCCTGCTCTTTCGGCGGCGTGAGCGTGATCA |
| | | CACCCGGCACCAATACAAGCAACCAGGTGGCCGTGCTGTATCAG |
| | | GACGTGAATTGTACCGAGGTGCCCGTGGCTATCCACGCCGATCAG |
| | | CTGACCCCAACATGGCGGGTGTACAGCACCGGCTCCAACGTCTTC |
| | | CAGACAAGAGCCGGATGCCTGATCGGAGCAGAGCACGTGAACAA |
| | | TTCCTATGAGTGCGACATCCCAATCGGCGCCGGCATCTGTGCCTC |
| | | TTACCAGACCCAGACAAACTCTCCCAGAAGAGCCCGGAGCGTGG |
| | | CCTCCCAGTCTATCATCGCCTATACCATGTCCCTGGGCGCCGAGA |
| | | ACAGCGTGGCCTACTCTAACAATAGCATCGCCATCCCAACCAACT |
| | | TCACAATCTCTGTGACCACAGAGATCCTGCCCGTGTCCATGACCA |
| | | AGACATCTGTGGACTGCACAATGTATATCTGTGGCGATTCTACCG |
| | | AGTGCAGCAACCTGCTGCTCCAGTACGGCAGCTTTTGTACCCAGC |
| | | TGAATAGAGCCCTGACAGGCATCGCCGTGGAGCAGGATAAGAAC |
| | | ACACAGGAGGTGTTCGCCCAGGTGAAGCAAATCTACAAGACCCC |
| | | CCCTATCAAGGACTTTGGCGGCTTCAATTTTTCCCAGATCCTGCCT |
| | | GATCCATCCAAGCCTTCTAAGCGGAGCTTTATCGAGGACCTGCTG |
| | | TTCAACAAGGTGACCCTGGCCGATGCCGGCTTCATCAAGCAGTAT |
| | | GGCGATTGCCTGGGCGACATCGCAGCCAGGGACCTGATCTGCGCC |
| | | CAGAAGTTTAATGGCCTGACCGTGCTGCCACCCCTGCTGACAGAT |
| | | GAGATGATCGCACAGTACACAAGCGCCCTGCTGGCCGGCACCAT |
| | | CACATCCGGATGGACCTTCGGCGCAGGAGCCGCCCTCCAGATCCC |
| | | CTTTGCCATGCAGATGGCCTATAGGTTCAACGGCATCGGCGTGAC |
| | | CCAGAATGTGCTGTACGAGAACCAGAAGCTGATCGCCAATCAGTT |
| | | TAACTCCGCCATCGGCAAGATCCAGGACAGCCTGTCCTCTACAGC |
| | | CAGCGCCCTGGGCAAGCTCCAGGATGTGGTGAATCAGAACGCCC |
| | | AGGCCCTGAATACCCTGGTGAAGCAGCTGAGCAGCAACTTCGGC |
| | | GCCATCTCTAGCGTGCTGAATGACATCCTGAGCCGGCTGGACAAG |
| | | GTGGAGGCAGAGGTGCAGATCGACCGGCTGATCACCGGCCGGCT |
| | | CCAGAGCCTCCAGACCTATGTGACACAGCAGCTGATCAGGGCCG |
| | | CCGAGATCAGGGCCAGCGCCAATCTGGCAGCAACCAAGATGTCC |
| | | GAGTGCGTGCTGGGCCAGTCTAAGAGAGTGGACTTTTGTGGCAAG |
| | | GGCTATCACCTGATGTCCTTCCCTCAGTCTGCCCCACACGGCGTG |
| | | GTGTTTCTGCACGTGACCTACGTGCCCGCCCAGGAGAAGAACTTC |
| | | ACCACAGCCCCTGCCATCTGCCACGATGGCAAGGCCCACTTTCCA |
| | | AGGGAGGGCGTGTTCGTGTCCAACGGCACCCACTGGTTTGTGACA |
| | | CAGCGCAATTTCTACGAGCCCCAGATCATCACCACAGACAACACC |
| | | TTCGTGAGCGGCAACTGTGACGTGGTCATCGGCATCGTGAACAAT |
| | | ACCGTGTATGATCCACTCCAGCCCGAGCTGGACAGCTTTAAGGAG |
| | | GAGCTGGATAAGTATTTCAAGAATCACACCTCCCCTGACGTGGAT |
| | | CTGGGCGACATCAGCGGCATCAATGCCTCCGTGGTGAACATCCAG |
| | | AAGGAGATCGACCGCCTGAACGAGGTGGCTAAGAATCTGAACGA |
| | | GAGCCTGATCGACCTCCAGGAGCTGGGCAAGTATGAGCAGTACA |
| | | TCAAGTGGCCCTGGTACATCTGGCTGGGCTTCATCGCCGGCCTGA |
| | | TCGCCATCGTGATGGTGACCATCATGCTGTGCTGTATGACATCCT |
| | | GCTGTTCTTGCCTGAAGGGCTGCTGTAGCTGTGGCTCCTGCTGTA |
| | | AGTTTGACGAGGATGACTCTGAACCTGTGCTGAAGGGCGTGAAG |
| | | CTGCATTACACCTAA |
| 2019-nCoV | amino acid | MFVFLVLLPLVSSQCVNLTTRTQLPPAYTNSFTRGVYYPDKVFRSSV |
| Spike gene | | LHSTQDLFLPFFSNVTWFHAIHVSGTNGTKRFDNPVLPFNDGVYFAS |
| (SEQ ID | | TEKSNIIRGWIFGTTLDSKTQSLLIVNNATNVVIKVCEFQFCNDPFLG |
| NO: 120) | | VYYHKNNKSWMESEFRVYSSANNCTFEYVSQPFLMDLEGKQGNFK |
| | | NLREFVFKNIDGYFKIYSKHTPINLVRDLPQGFSALEPLVDLPIGINITR |
| | | FQTLLALHRSYLTPGDSSSGWTAGAAAYYVGYLQPRTFLLKYNENG |
| | | TITDAVDCALDPLSETKCTLKSFTVEKGIYQTSNFRVQPTESIVRFPNI |
| | | TNLCPFGEVFNATRFASVYAWNRKRISNCVADYSVLYNSASFSTFKC |
| | | YGVSPTKLNDLCFTNVYADSFVIRGDEVRQIAPGQTGKIADYNYKLP |
| | | DDFTGCVIAWNSNNLDSKVGGNYNYLYRLFRKSNLKPFERDISTEIY |
| | | QAGSTPCNGVEGFNCYFPLQSYGFQPTNGVGYQPYRVVVLSFELLH |
| | | APATVCGPKKSTNLVKNKCVNFNFNGLTGTGVLTESNKKFLPFQQF |
| | | GRDIADTTDAVRDPQTLEILDITPCSFGGVSVITPGTNTSNQVAVLYQ |
| | | DVNCTEVPVAIHADQLTPTWRVYSTGSNVFQTRAGCLIGAEHVNNS |
| | | YECDIPIGAGICASYQTQTNSPRRARSVASQSIIAYTMSLGAENSVAY |
| | | SNNSIAIPTNFTISVTTEILPVSMTKTSVDCTMYICGDSTECSNLLLQY |
| | | GSFCTQLNRALTGIAVEQDKNTQEVFAQVKQIYKTPPIKDFGGFNFS |
| | | QILPDPSKPSKRSFIEDLLFNKVTLADAGFIKQYGDCLGDIAARDLICA |
| | | QKFNGLTVLPPLLTDEMIAQYTSALLAGTITSGWTFGAGAALQIPFA |
| | | MQMAYRFNGIGVTQNVLYENQKLIANQFNSAIGKIQDSLSSTASALG |
| | | KLQDVVNQNAQALNTLVKQLSSNFGAISSVLNDILSRLDKVEAEVQI |
| | | DRLITGRLQSLQTYVTQQLIRAAEIRASANLAATKMSECVLGQSKRV |
| | | DFCGKGYHLMSFPQSAPHGVVFLHVTYVPAQEKNFTTAPAICHDGK |
| | | AHFPREGVFVSNGTHWFVTQRNFYEPQUITTDNTFVSGNCDVVIGIVN |
| | | NTVYDPLQPELDSFKEELDKYFKNHTSPDVDLGDISGINASVVNIQKE |
| | | IDRLNEVAKNLNESLIDLQELGKYEQYIKWPWYIWLGFIAGLIAIVM |
| | | VTIMLCCMTSCCSCLKGCCSCGSCCKFDEDDSEPVLKGVKLHYT* |

RNA sequences can include any combination of the RNA sequences listed in Tables 4 and 5. In some embodiments, RNA sequences of the present disclosure include any combination of the RNA sequences listed in Tables 4 and 5 in which 0% to 100%, 1% to 100%, 25% to 100%, 50% to 100% and 75% to 100% of the uracil nucleotides of the mRNA sequences are modified. In some embodiments, 1% to 100% of the uracil nucleotides are N1-methylpseudouridine or 5-methoxyuridine. In some embodiments, 100% of the uracil nucleotides are N1-methylpseudouridine. In some embodiments, 100% of the uracil nucleotides are 5-methoxyuridine.

A self-replicating RNA of the disclosure may be obtained by any suitable means. Methods for the manufacture of self-replicating RNA are known in the art and would be readily apparent to a person of ordinary skill. A self-replicating RNA of the disclosure may be prepared according to any available technique including, but not limited to chemical synthesis, in vitro transcription (IVT) or enzymatic or chemical cleavage of a longer precursor, etc.

In some embodiments, a self-replicating RNA of the disclosure is produced from a primary complementary DNA (cDNA) construct. The cDNA constructs can be produced on an RNA template by the action of a reverse transcriptase (e.g., RNA-dependent DNA-polymerase). The process of design and synthesis of the primary cDNA constructs described herein generally includes the steps of gene construction, RNA production (either with or without modifications) and purification. In the IVT method, a target polynucleotide sequence encoding a self-replicating RNA of the disclosure is first selected for incorporation into a vector which will be amplified to produce a cDNA template. Optionally, the target polynucleotide sequence and/or any flanking sequences may be codon optimized. The cDNA template is then used to produce a self-replicating RNA of the disclosure through in vitro transcription (IVT). After production, the self-replicating RNA of the disclosure may undergo purification and clean-up processes. The steps of which are provided in more detail below.

The step of gene construction may include, but is not limited to gene synthesis, vector amplification, plasmid purification, plasmid linearization and clean-up, and cDNA template synthesis and clean-up. Once a protein of interest is selected for production, a primary construct is designed. Within the primary construct, a first region of linked nucleosides encoding the polypeptide of interest may be constructed using an open reading frame (ORF) of a selected nucleic acid (DNA or RNA) transcript. The ORF may comprise the wild type ORF, an isoform, variant or a fragment thereof. As used herein, an “open reading frame” or “ORF” is meant to refer to a nucleic acid sequence (DNA or RNA) which is capable of encoding a polypeptide of interest. ORFs often begin with the start codon, ATG and end with a nonsense or termination codon or signal.

The cDNA templates may be transcribed to produce a self-replicating RNA of the disclosure using an in vitro transcription (IVT) system. The system typically comprises a transcription buffer, nucleotide triphosphates (NTPs), an RNase inhibitor and a polymerase. The NTPs may be selected from, but are not limited to, those described herein including natural and unnatural (modified) NTPs. The polymerase may be selected from, but is not limited to, T7 RNA polymerase, T3 RNA polymerase and mutant polymerases such as, but not limited to, polymerases able to incorporate modified nucleic acids.

The primary cDNA template or transcribed RNA sequence may also undergo capping and/or tailing reactions. A capping reaction may be performed by methods known in the art to add a 5′ cap to the 5′ end of the primary construct. Methods for capping include, but are not limited to, using a Vaccinia Capping enzyme (New England Biolabs, Ipswich, Mass.) or capping at initiation of in vitro transcription, by for example, including a capping agent as part of the IVT reaction. (Nuc. Acids Symp. (2009) 53:129). A poly(A) tailing reaction may be performed by methods known in the art, such as, but not limited to, 2′ O-methyltransferase and by methods as described herein. If the primary construct generated from cDNA does not include a poly-T, it may be beneficial to perform the poly(A)-tailing reaction before the primary construct is cleaned.

Codon optimized cDNA constructs encoding the non-structural proteins and the transgene for a self-replicating RNA protein are particularly suitable for generating self-replicating RNA sequences described herein. For example, such cDNA constructs may be used as the basis to transcribe, in vitro, a polyribonucleotide encoding a protein of interest as part of a self-replicating RNA.

The present disclosure also provides expression vectors comprising a nucleotide sequence encoding a self-replicating RNA that is preferably operably linked to at least one regulatory sequence. Regulatory sequences are art-recognized and are selected to direct expression of the encoded polypeptide.

Accordingly, the term regulatory sequence includes promoters, enhancers, and other expression control elements. The design of the expression vector may depend on such factors as the choice of the host cell to be transformed and/or the type of protein desired to be expressed.

The present disclosure also provides polynucleotides (e.g. DNA, RNA, cDNA, mRNA, etc.) directed to a self-replicating RNA of the disclosure that may be operably linked to one or more regulatory nucleotide sequences in an expression construct, such as a vector or plasmid. In certain embodiments, such constructs are DNA constructs. Regulatory nucleotide sequences will generally be appropriate for a host cell used for expression. Numerous types of appropriate expression vectors and suitable regulatory sequences are known in the art for a variety of host cells.

Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the embodiments of the present disclosure. The promoters may be either naturally occurring promoters, or hybrid promoters that combine elements of more than one promoter.

An expression construct may be present in a cell on an episome, such as a plasmid, or the expression construct may be inserted in a chromosome. In some embodiments, the expression vector contains a selectable marker gene to allow the selection of transformed host cells. Selectable marker genes are well known in the art and will vary with the host cell used.

The present disclosure also provides a host cell transfected with a self-replicating RNA or DNA described herein. The self-replicating RNA or DNA can encode any coronavirus protein of interest, for example an antigen, including the S-antigen of the COVID-19 virus. The host cell may be any prokaryotic or eukaryotic cell. For example, a polypeptide encoded by a self-replicating RNA may be expressed in bacterial cells such as E. coli, insect cells (e.g., using a baculovirus expression system), yeast, or mammalian cells. Other suitable host cells are known to those skilled in the art.

A host cell transfected with an expression vector comprising a self-replicating RNA of the disclosure can be cultured under appropriate conditions to allow expression of the amplification of the self-replicating RNA and translation of the polypeptide to occur. The polypeptide may be secreted and isolated from a mixture of cells and medium containing the polypeptides. Alternatively, the polypeptides may be retained in the cytoplasm or in a membrane fraction and the cells harvested, lysed and the protein isolated. A cell culture includes host cells, media and other byproducts. Suitable media for cell culture are well known in the art.

The expressed proteins described herein can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the polypeptide.

### Compositions and Pharmaceutical Compositions

Provided herein, in some embodiments, are compositions comprising any of the nucleic acid molecules provided herein. Compositions provided herein can include a lipid. Any lipid can be included in compositions provided herein. In one aspect, the lipid is an ionizable cationic lipid. Any ionizable cationic lipid can be included in compositions comprising nucleic acid molecules provided herein.

The compositions and polynucleotides of the present disclosure may be used to immunize or vaccinate a subject against a viral infection. In some embodiments, the compositions and polynucleotides of the present disclosure may be used to vaccinate or immunize a subject against COVID-19 virus.

Also provided herein, in some embodiments, are pharmaceutical compositions comprising any of the nucleic acid molecules provided herein and a lipid formulation. Any lipid can be included in lipid formulations of pharmaceutical compositions provided herein. In one aspect, lipid formulations of pharmaceutical compositions provided herein include an ionizable cationic lipid. Exemplary ionizable cationic lipids of compositions and pharmaceutical compositions provided herein include the following:

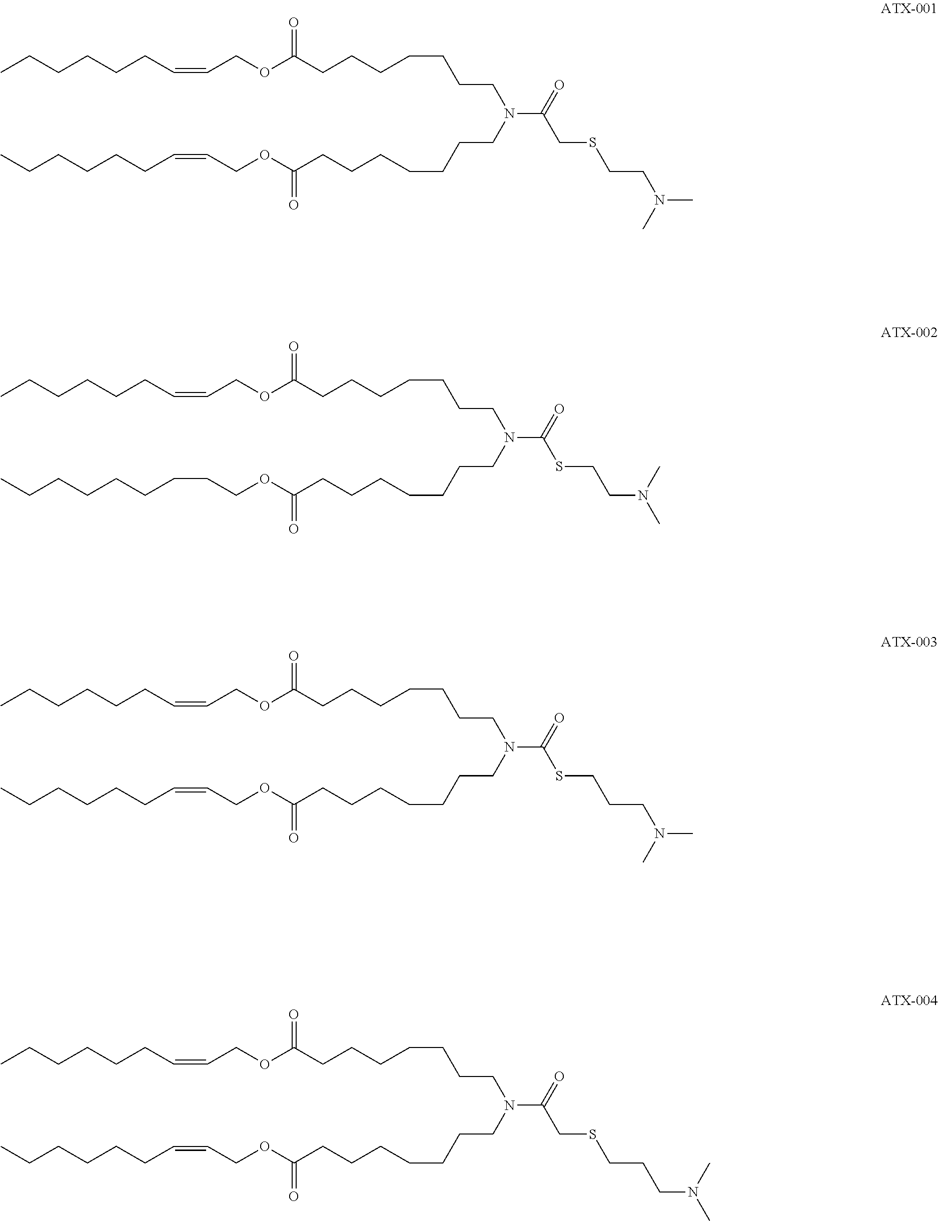

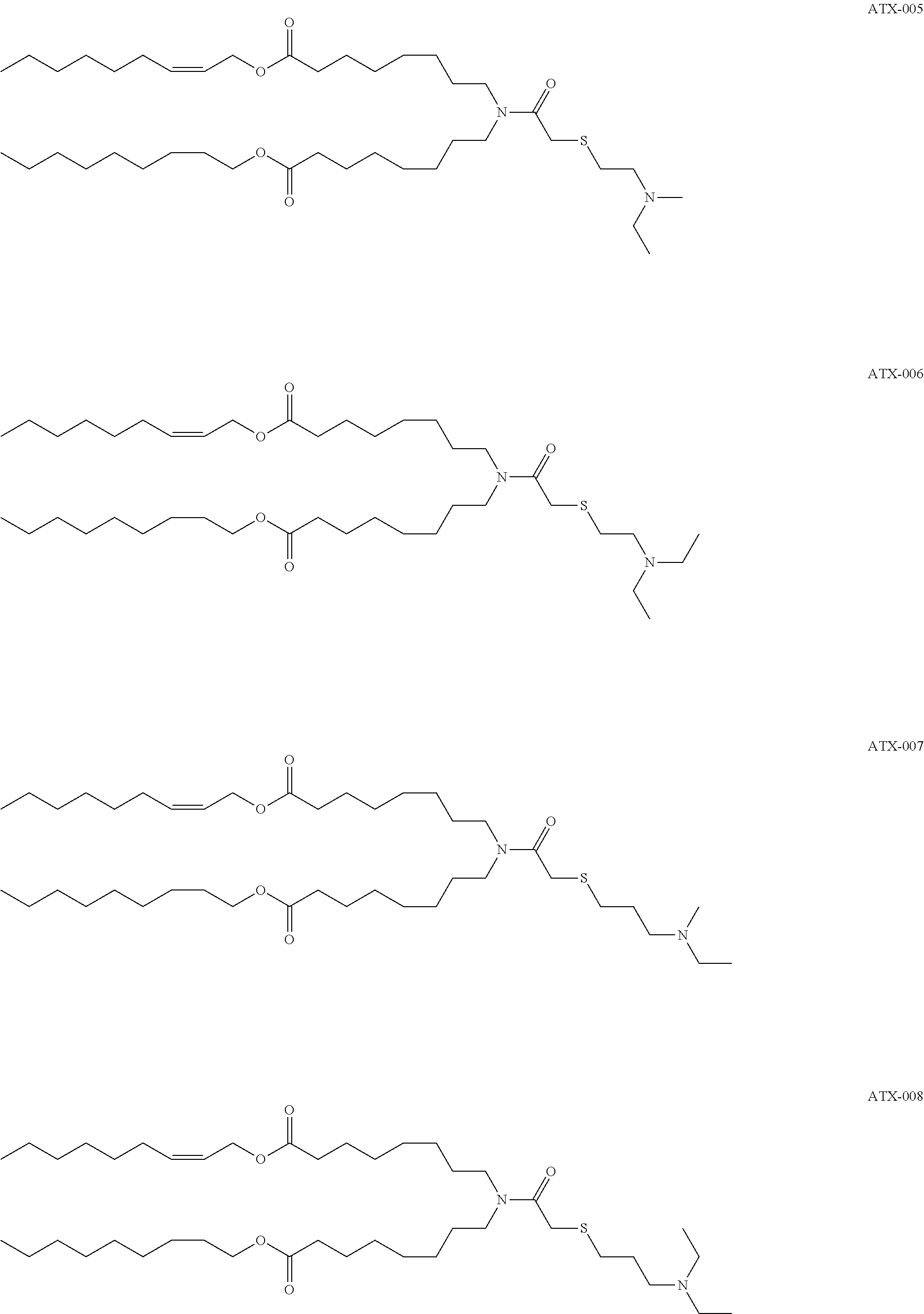

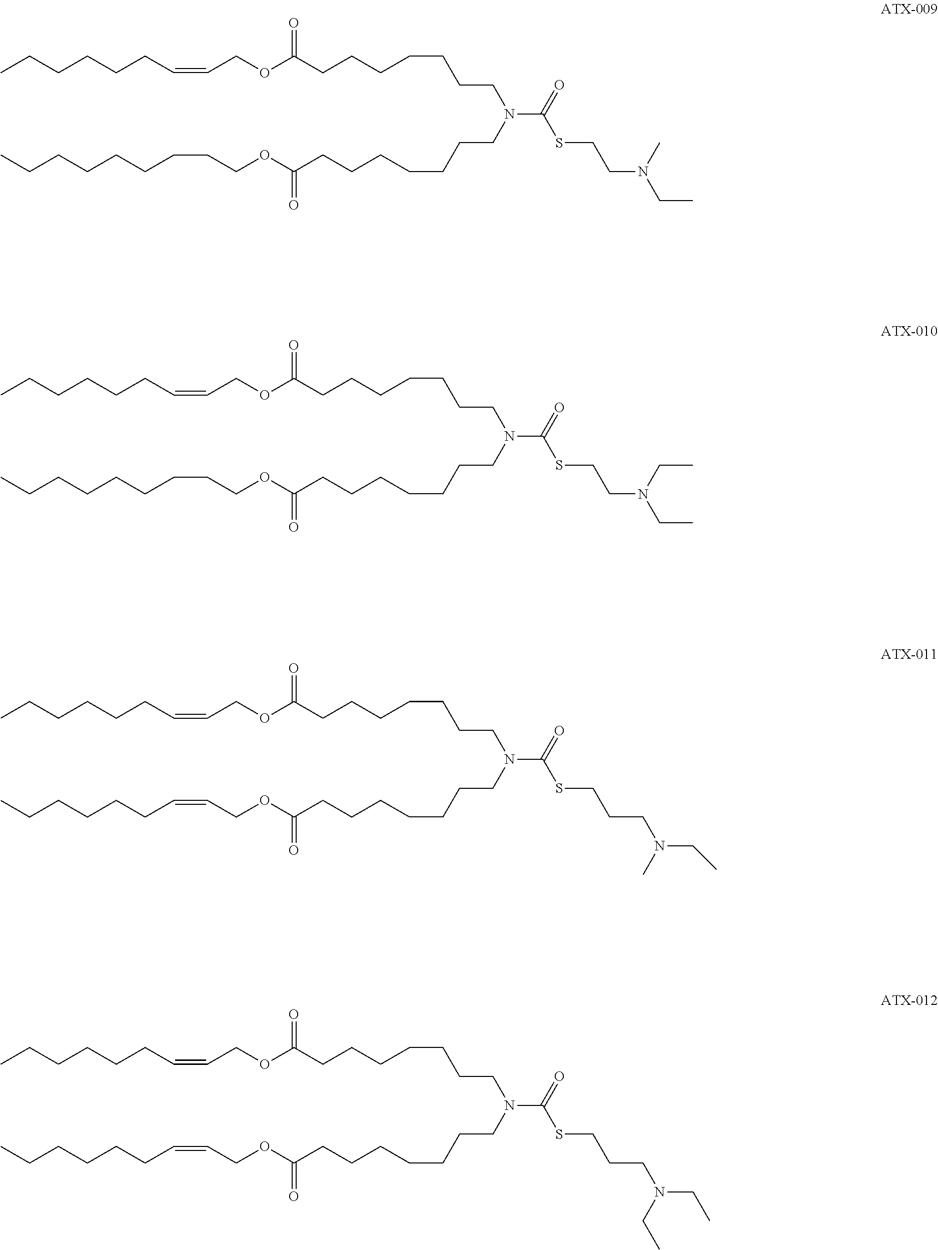

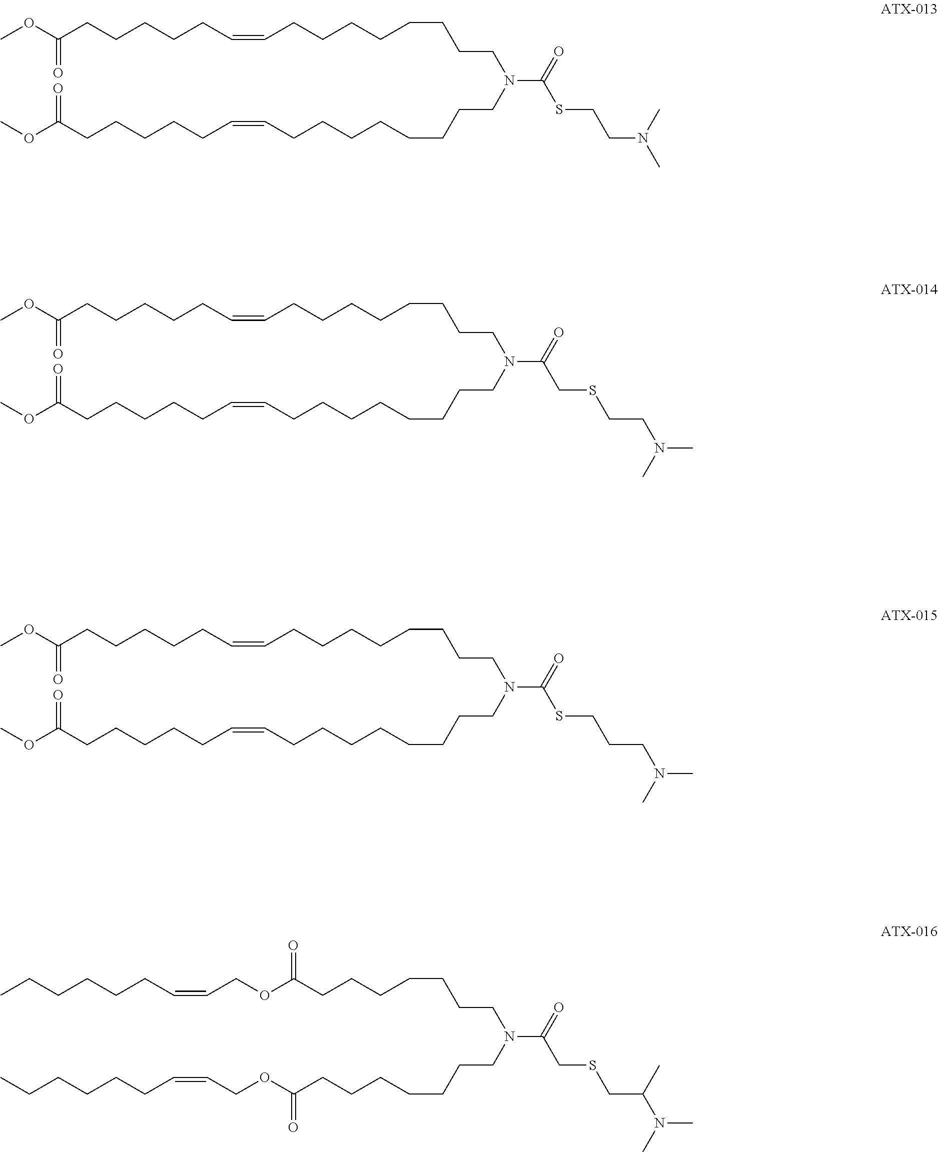

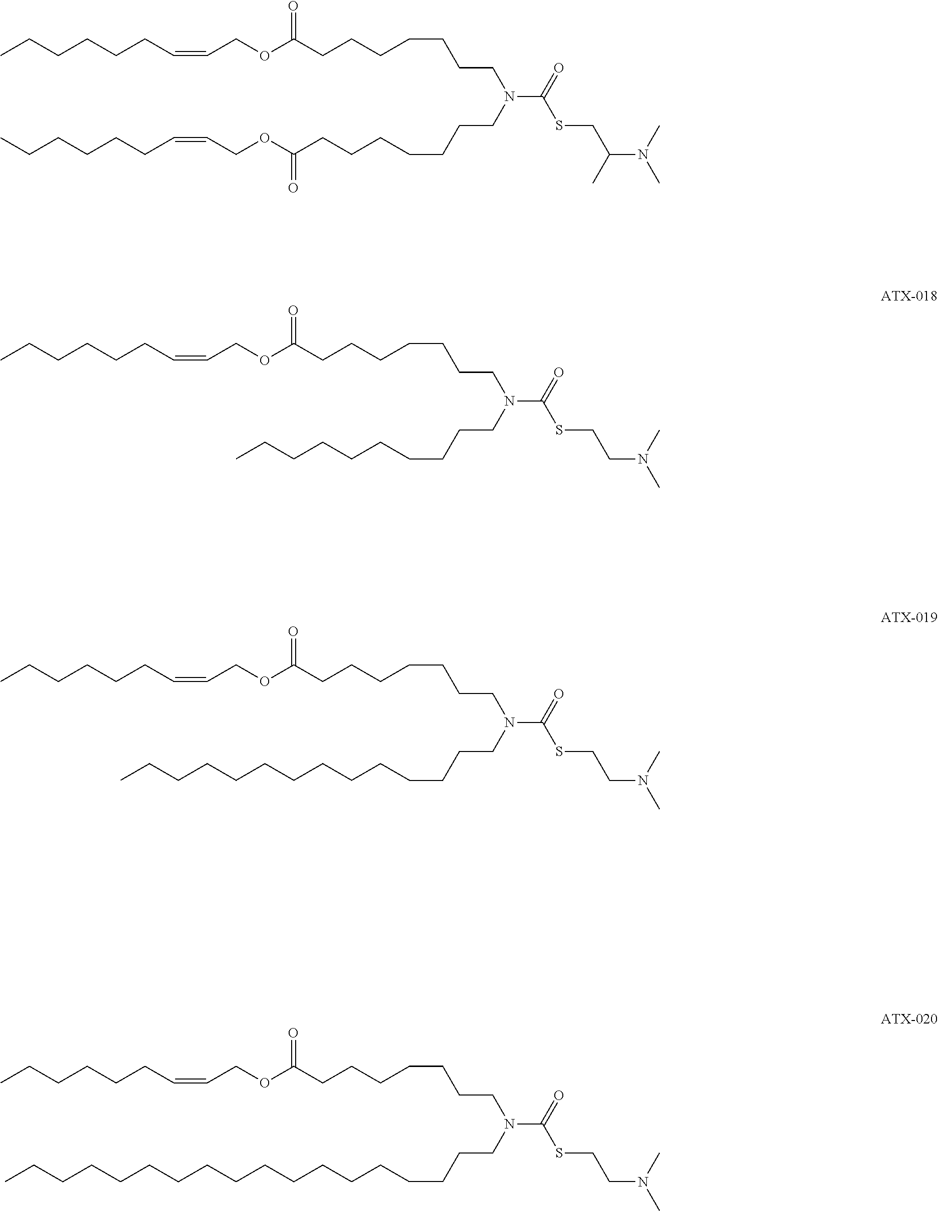

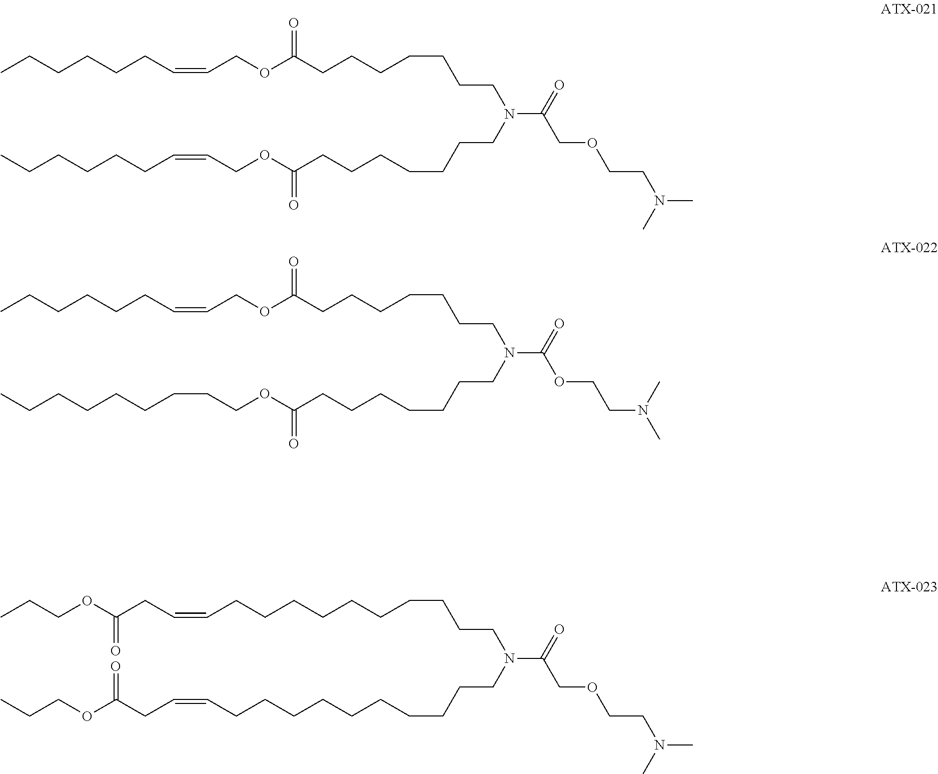

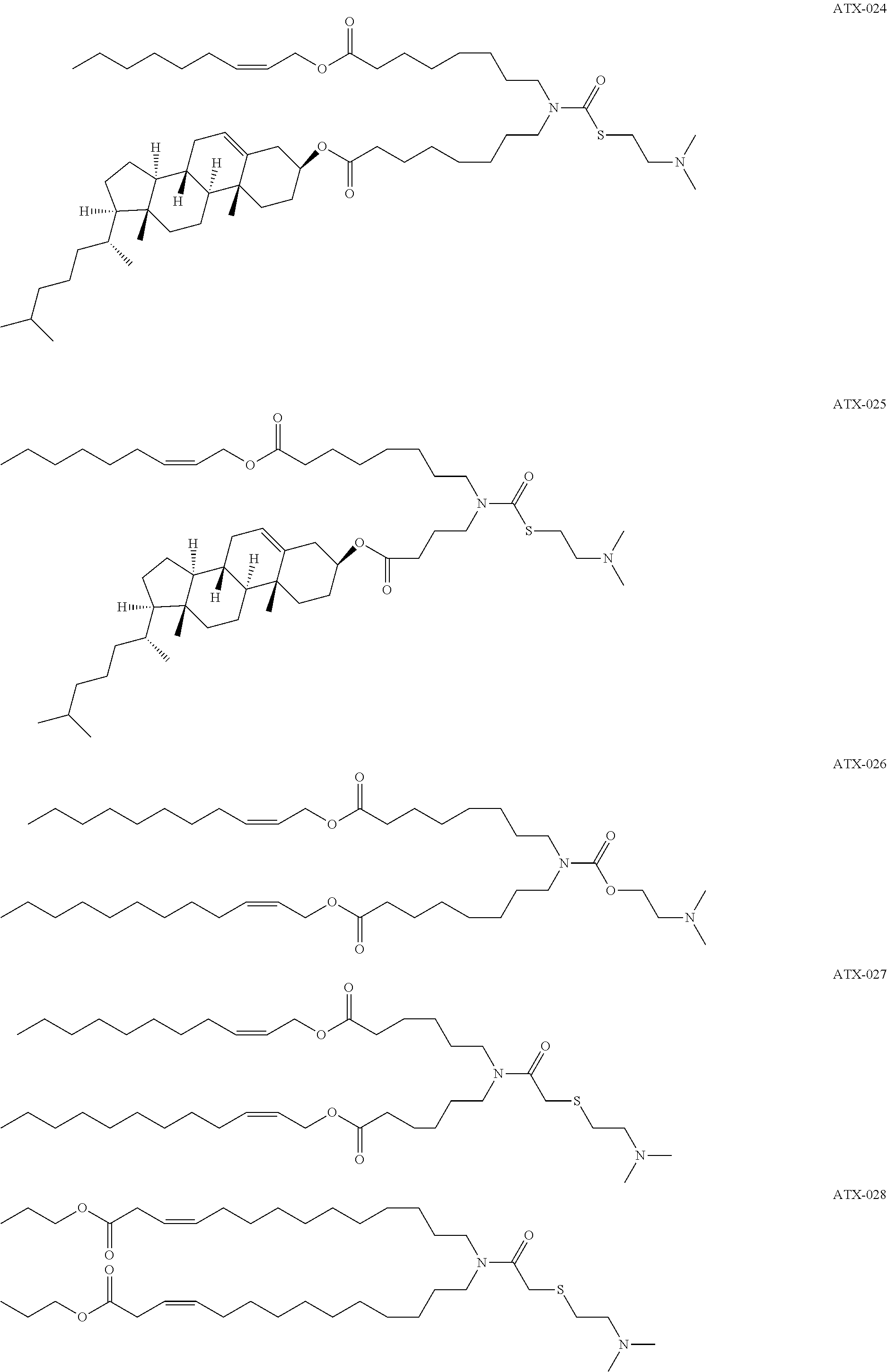

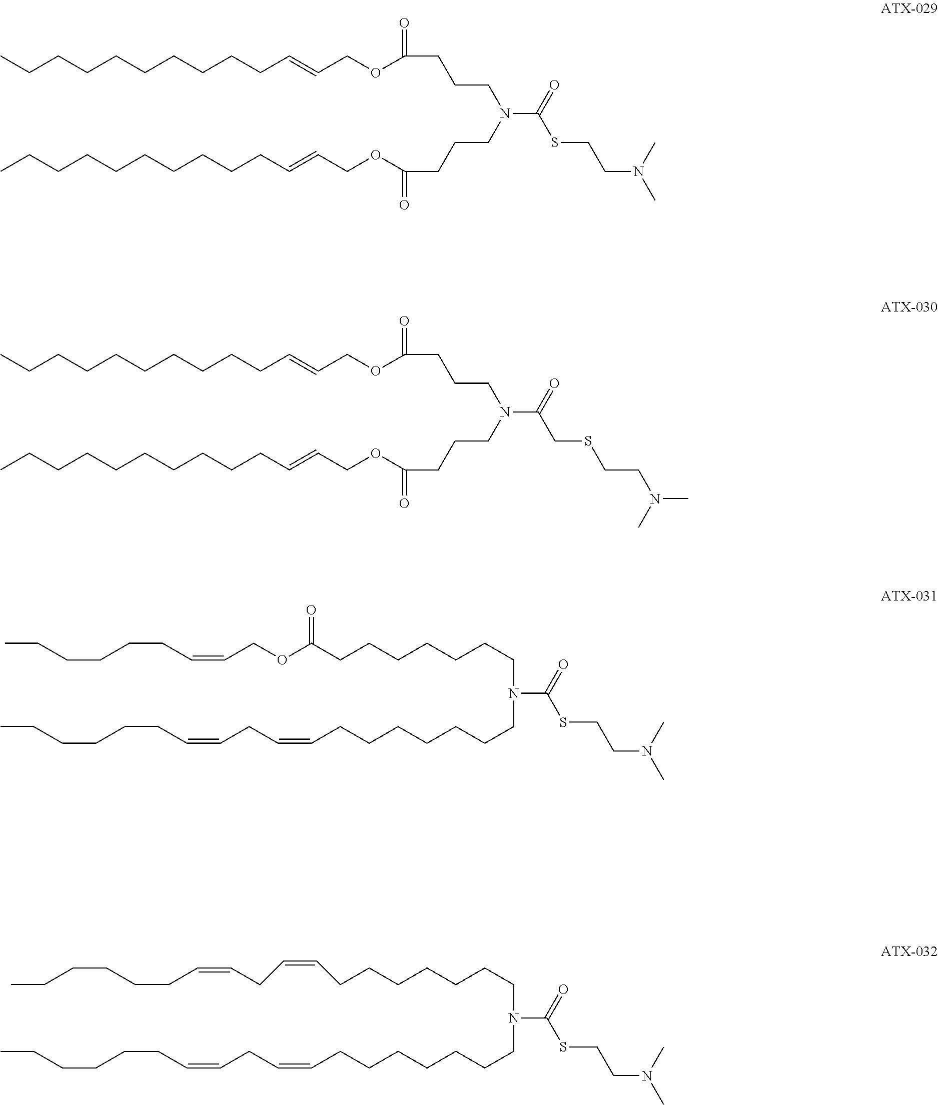

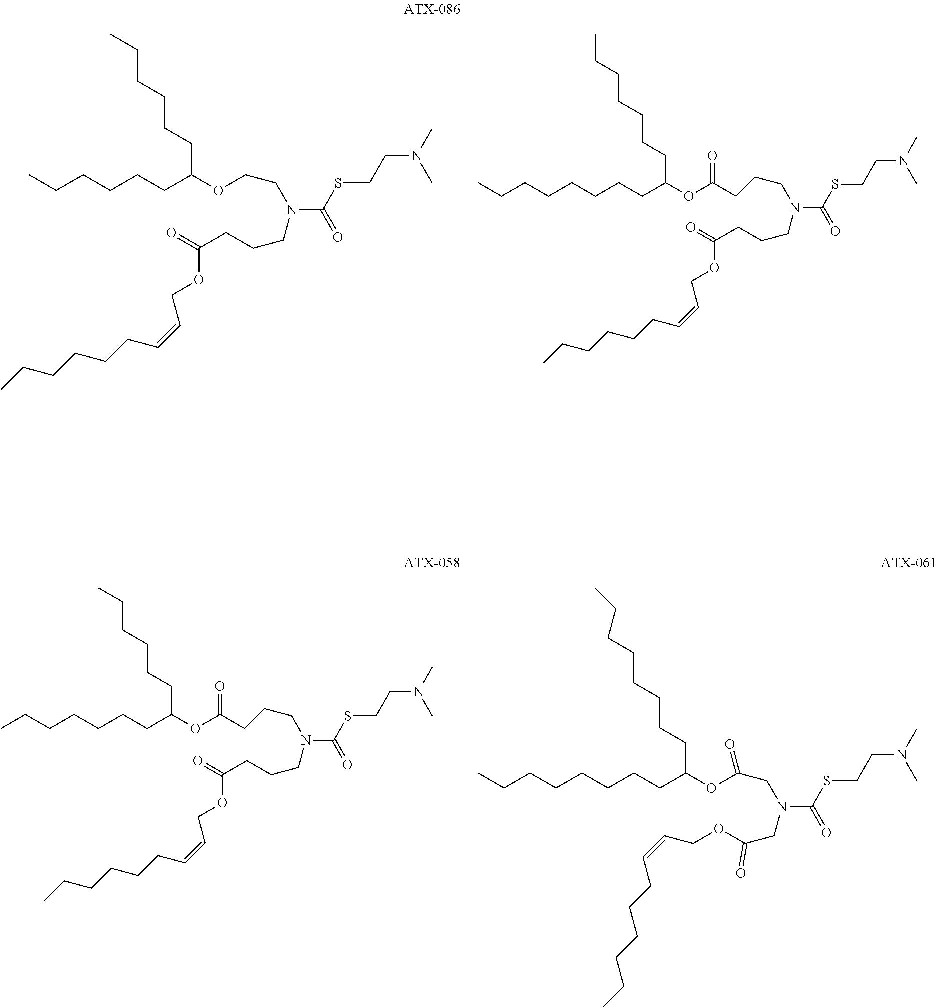 [SMILES: CCCCCC/C=C\COC(=O)CCCN(CCOC(CCCCCC)CCCCCC)C(=O)SCCN(C)C ; CCCCCC/C=C\COC(=O)CCCN(CCCC(=O)OC(CCCCCCC)CCCCCCCC)C(=O)SCCN(C)C ; CCCCCC/C=C\COC(=O)CCCN(CCCC(=O)OC(CCCCCC)CCCCCCC)C(=O)SCCN(C)C ; CCCCCC/C=C\COC(=O)CN(CC(=O)OC(CCCCCCCC)CCCCCCCC)C(=O)SCCN(C)C]

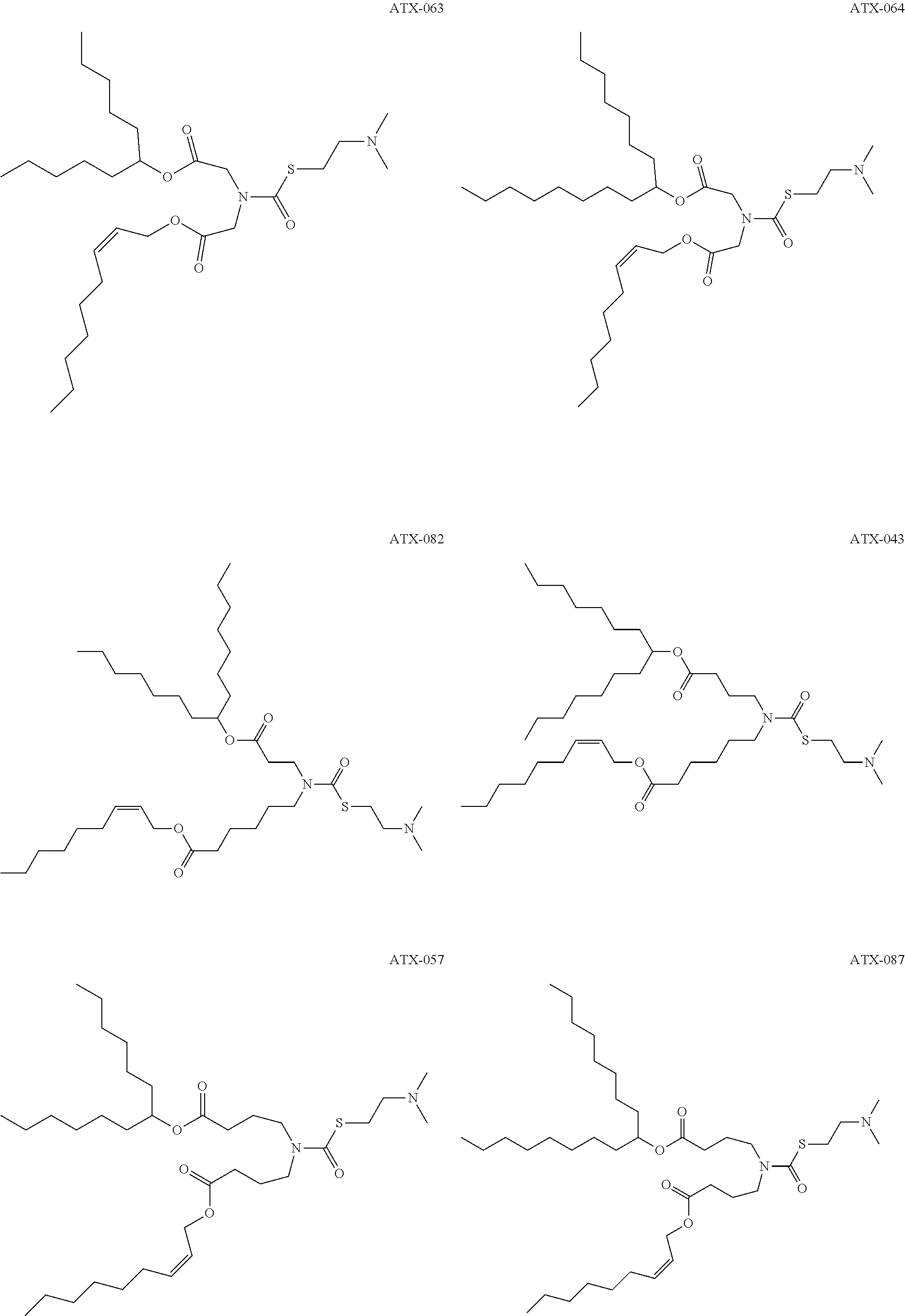 [SMILES: CCCCCC/C=C\COC(=O)CN(CC(=O)OC(CCCCC)CCCCC)C(=O)SCCN(C)C ; CCCCCC/C=C\COC(=O)CN(CC(=O)OC(CCCCCCC)CCCCCCCC)C(=O)SCCN(C)C ; CCCCCC/C=C\COC(=O)CCCCCN(CCC(=O)OC(CCCCCCC)CCCCCCC)C(=O)SCCN(C)C ; CCCCCC/C=C\COC(=O)CCCCCN(CCCC(=O)OC(CCCCCCC)CCCCCCC)C(=O)SCCN(C)C ; CCCCCC/C=C\COC(=O)CCCN(CCCC(=O)OC(CCCCCC)CCCCCC)C(=O)SCCN(C)C ; CCCCCC/C=C\COC(=O)CCCN(CCCC(=O)OC(CCCCCCCC)CCCCCCCC)C(=O)SCCN(C)C]

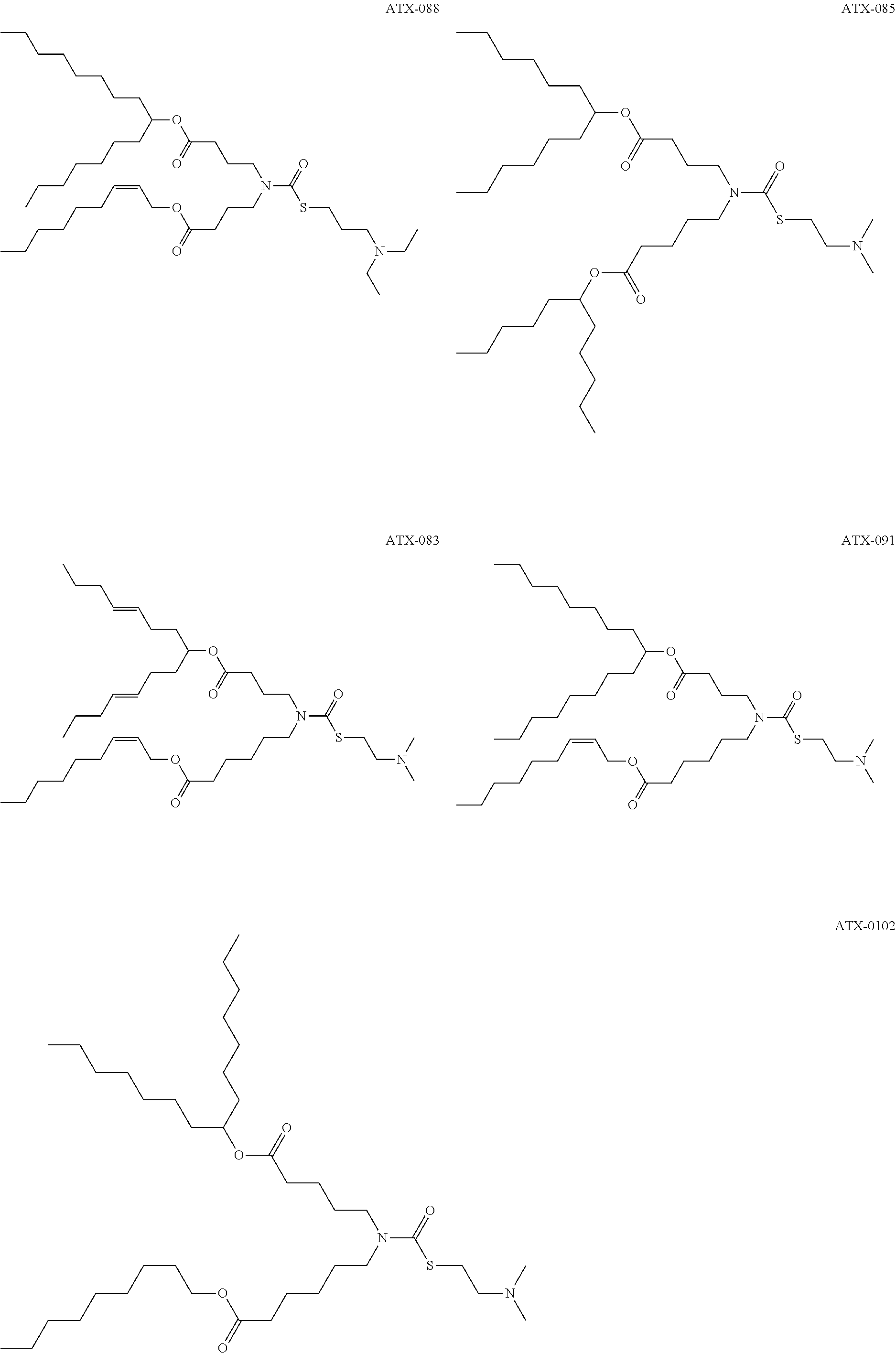 [SMILES: CCCCCC/C=C\COC(=O)CCCN(CCCC(=O)OC(CCCCCCC)CCCCCCCC)C(=O)SCCCN(CC)CC ; CCCCCCC(CCCCCC)OC(=O)CCCN(CCCCC(=O)OC(CCCCC)CCCCC)C(=O)SCCN(C)C ; CCC/C=C/CCC(CC/C=C/CCC)OC(=O)CCCN(CCCCCC(=O)OC/C=C\CCCCCC)C(=O)SCCN(C)C ; CCCCCC/C=C\COC(=O)CCCCCN(CCCC(=O)OC(CCCCCCCC)CCCCCCCC)C(=O)SCCN(C)C ; CCCCCCCCCOC(=O)CCCCCN(CCCCC(=O)OC(CCCCCCC)CCCCCCC)C(=O)SCCN(C)C]

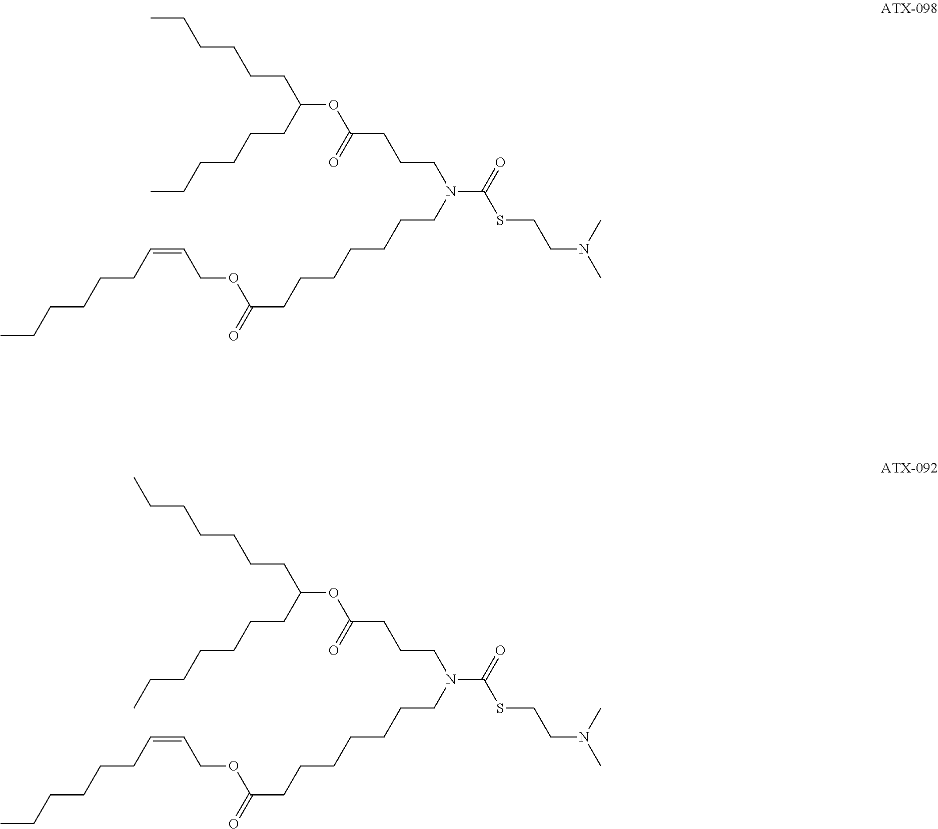

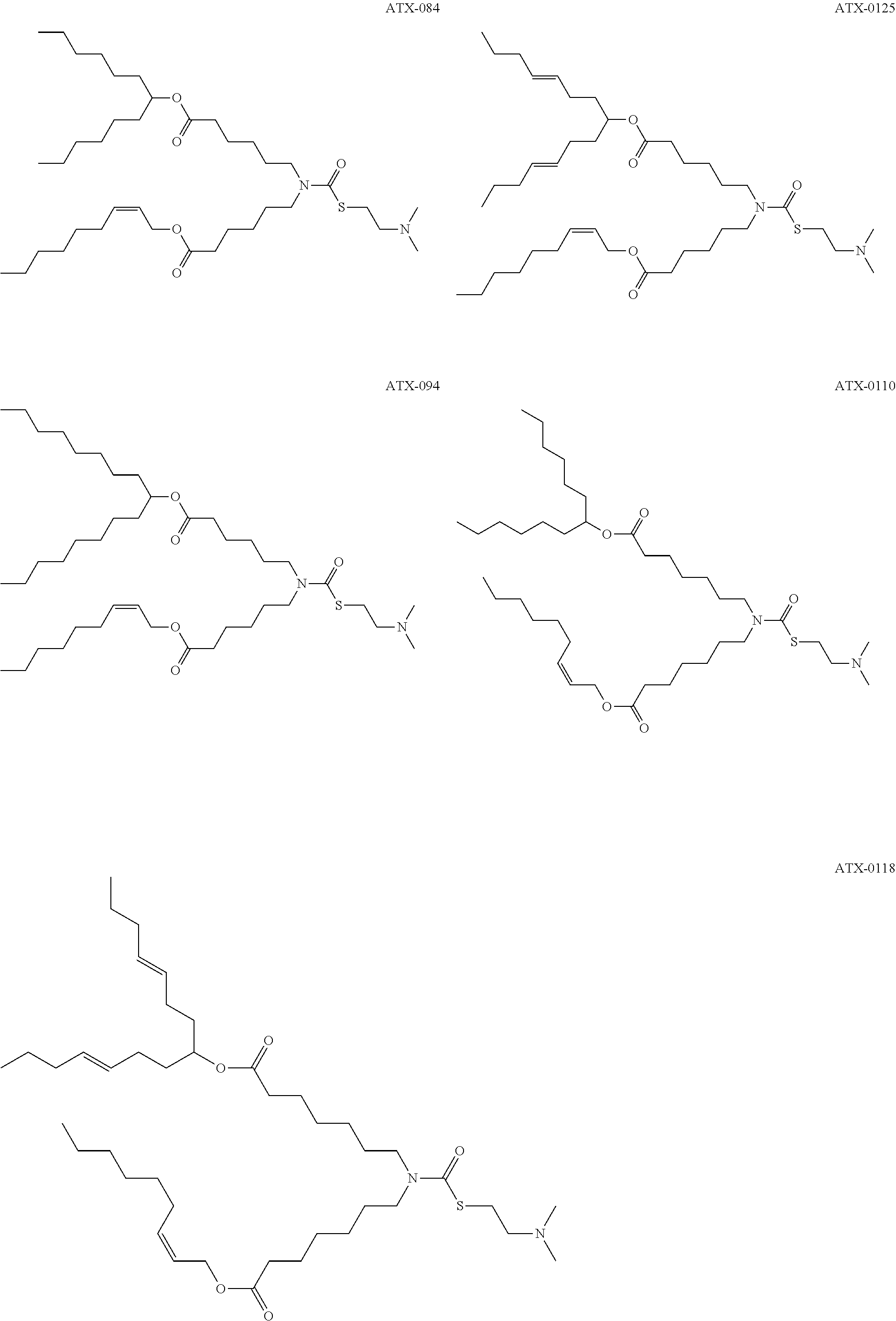 [SMILES: CCCCCC/C=C\COC(=O)CCCCCN(CCCCCC(=O)OC(CCCCCC)CCCCCC)C(=O)SCCN(C)C ; CCC/C=C/CCC(CC/C=C/CCC)OC(=O)CCCCCN(CCCCCC(=O)OC/C=C\CCCCCC)C(=O)SCCN(C)C ; CCCCCC/C=C\COC(=O)CCCCCN(CCCCCC(=O)OC(CCCCCCCC)CCCCCCCC)C(=O)SCCN(C)C ; CCCCCC/C=C\COC(=O)CCCCCCN(CCCCCCC(=O)OC(CCCCCC)CCCCCC)C(=O)SCCN(C)C ; CCC/C=C/CCC(CC/C=C/CCC)OC(=O)CCCCCCN(CCCCCCC(=O)OC/C=C\CCCCCC)C(=O)SCCN(C)C]

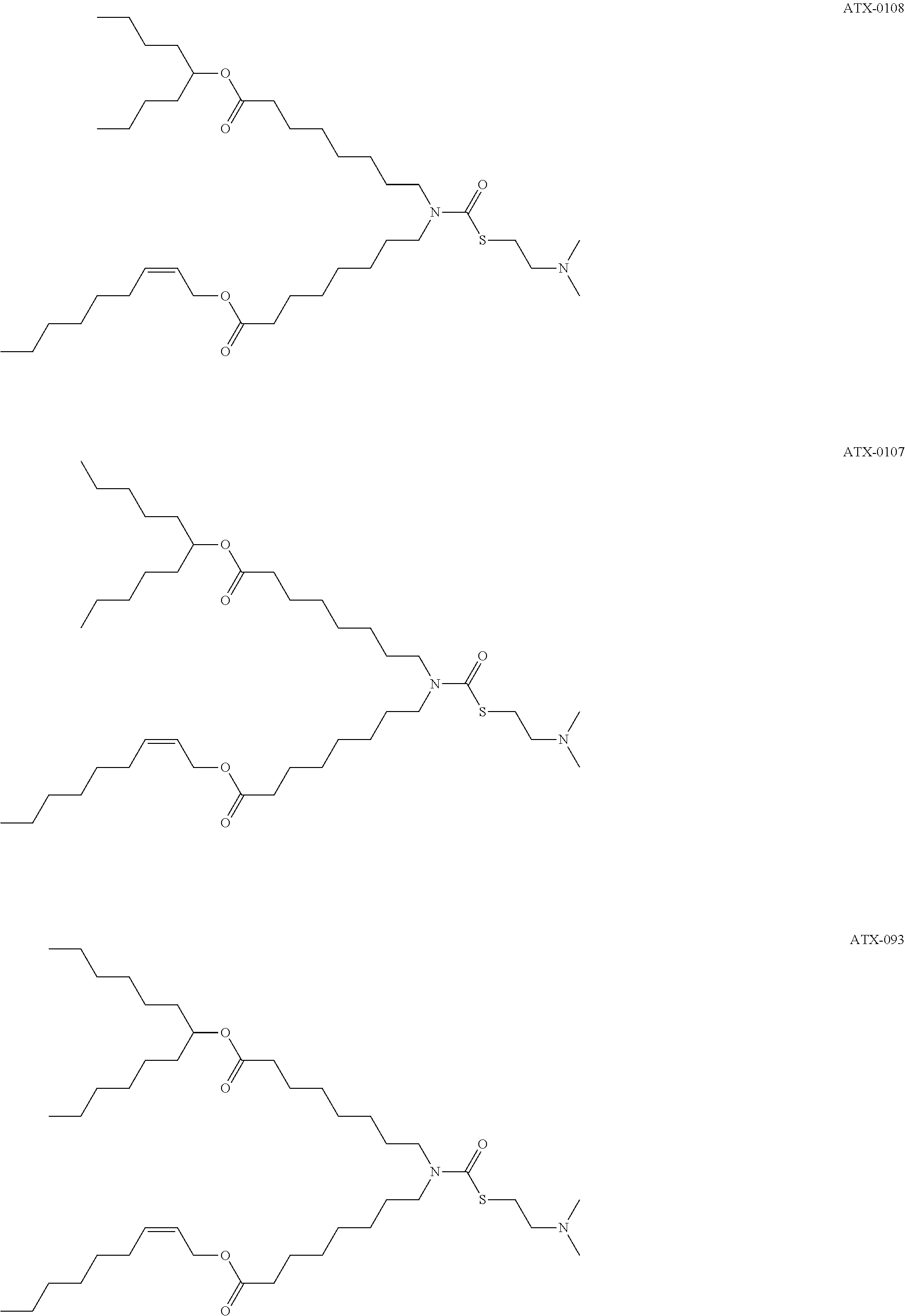

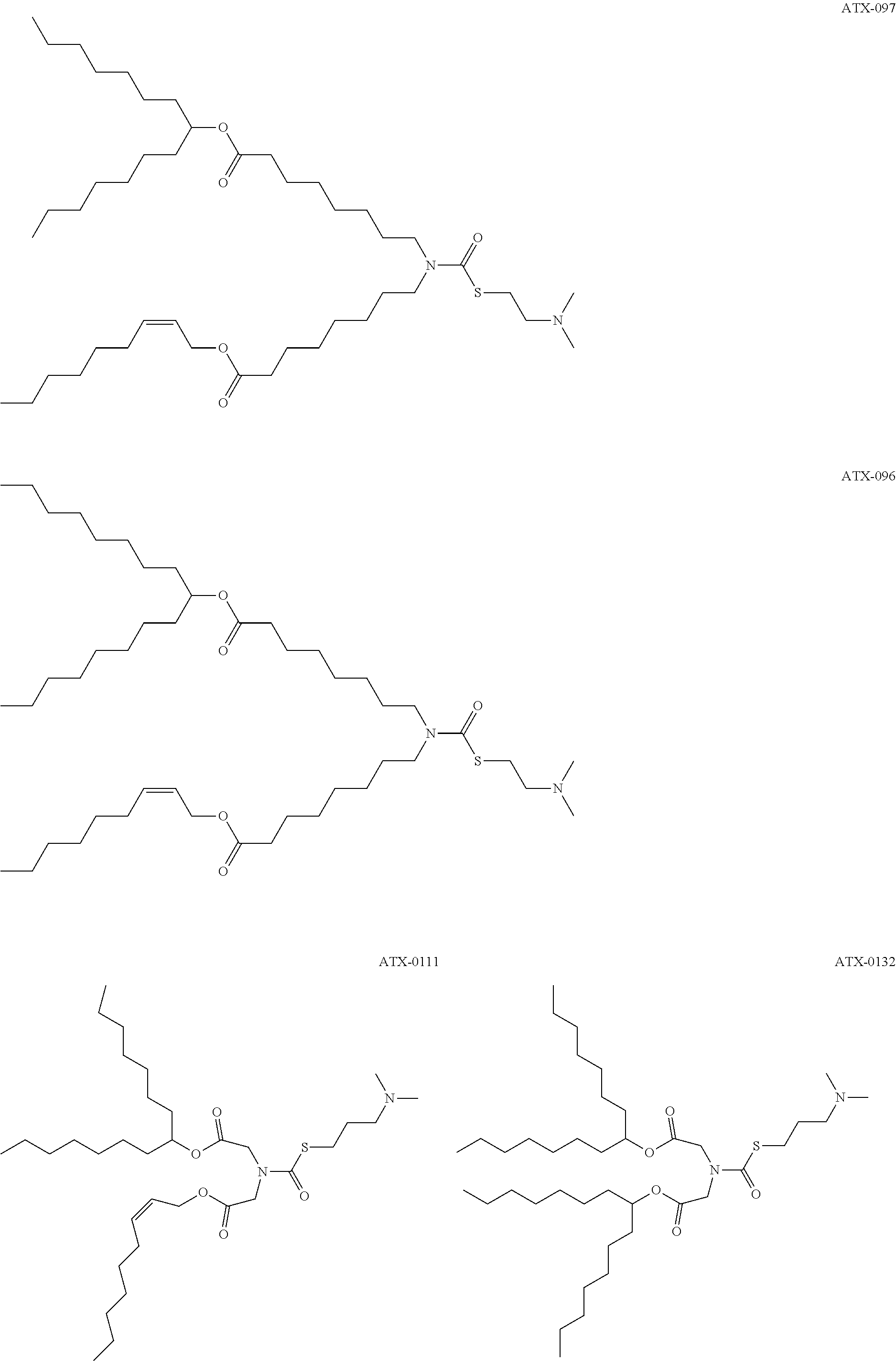

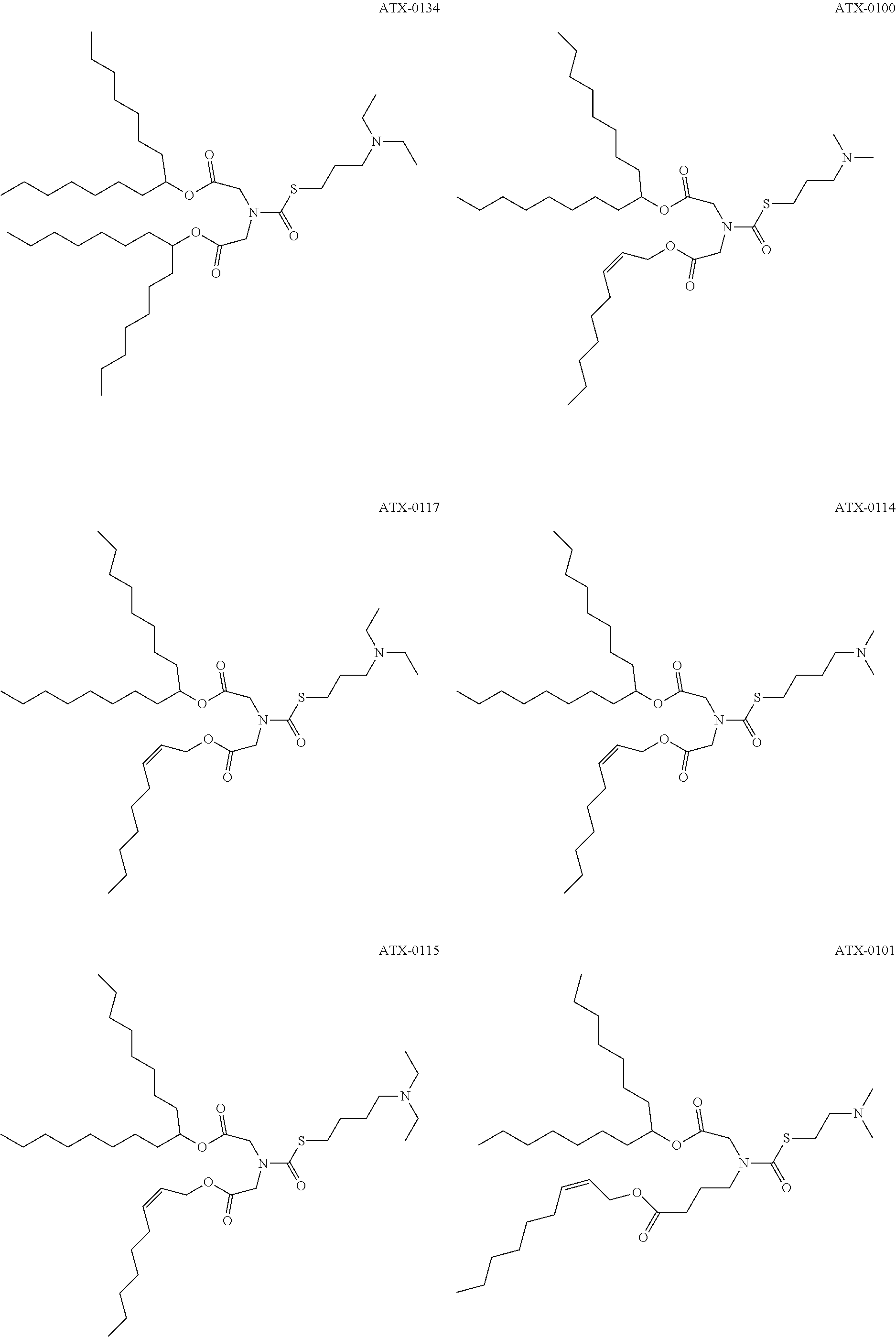 [SMILES: CCCCCCCC(CCCCCCC)OC(=O)CN(CC(=O)OC(CCCCCCC)CCCCCCC)C(=O)SCCCN(CC)CC ; CCCCCC/C=C\COC(=O)CN(CC(=O)OC(CCCCCCCC)CCCCCCCC)C(=O)SCCCN(C)C ; CCCCCC/C=C\COC(=O)CN(CC(=O)OC(CCCCCCCC)CCCCCCCC)C(=O)SCCCN(CC)CC ; CCCCCC/C=C\COC(=O)CN(CC(=O)OC(CCCCCCCC)CCCCCCCC)C(=O)SCCCCN(C)C ; CCCCCC/C=C\COC(=O)CN(CC(=O)OC(CCCCCCCC)CCCCCCCC)C(=O)SCCCCN(CC)CC ; CCCCCC/C=C\COC(=O)CCCN(CC(=O)OC(CCCCCCC)CCCCCCC)C(=O)SCCN(C)C]

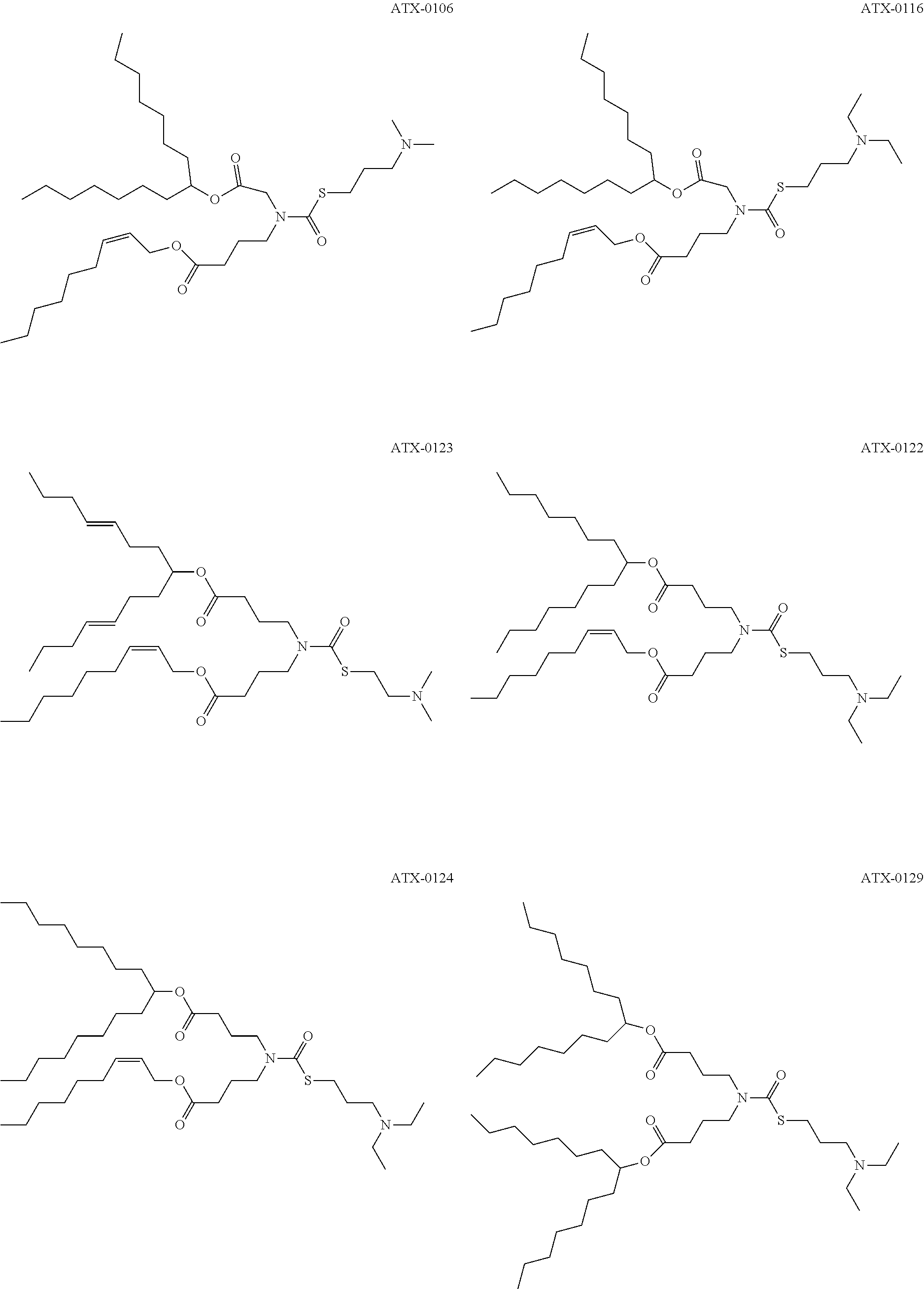 [SMILES: CCCCCC/C=C\COC(=O)CCCN(CC(=O)OC(CCCCCCC)CCCCCCC)C(=O)SCCCN(C)C ; CCCCCC/C=C\COC(=O)CCCN(CC(=O)OC(CCCCCCC)CCCCCCC)C(=O)SCCCN(CC)CC ; CCC/C=C/CCC(CC/C=C/CCC)OC(=O)CCCN(CCCC(=O)OC/C=C\CCCCCC)C(=O)SCCN(C)C ; CCCCCC/C=C\COC(=O)CCCN(CCCC(=O)OC(CCCCCCC)CCCCCCC)C(=O)SCCCN(CC)CC ; CCCCCC/C=C\COC(=O)CCCN(CCCC(=O)OC(CCCCCCCC)CCCCCCCC)C(=O)SCCCN(CC)CC ; CCCCCCCC(CCCCCCC)OC(=O)CCCN(CCCC(=O)OC(CCCCCCC)CCCCCCC)C(=O)SCCCN(CC)CC]

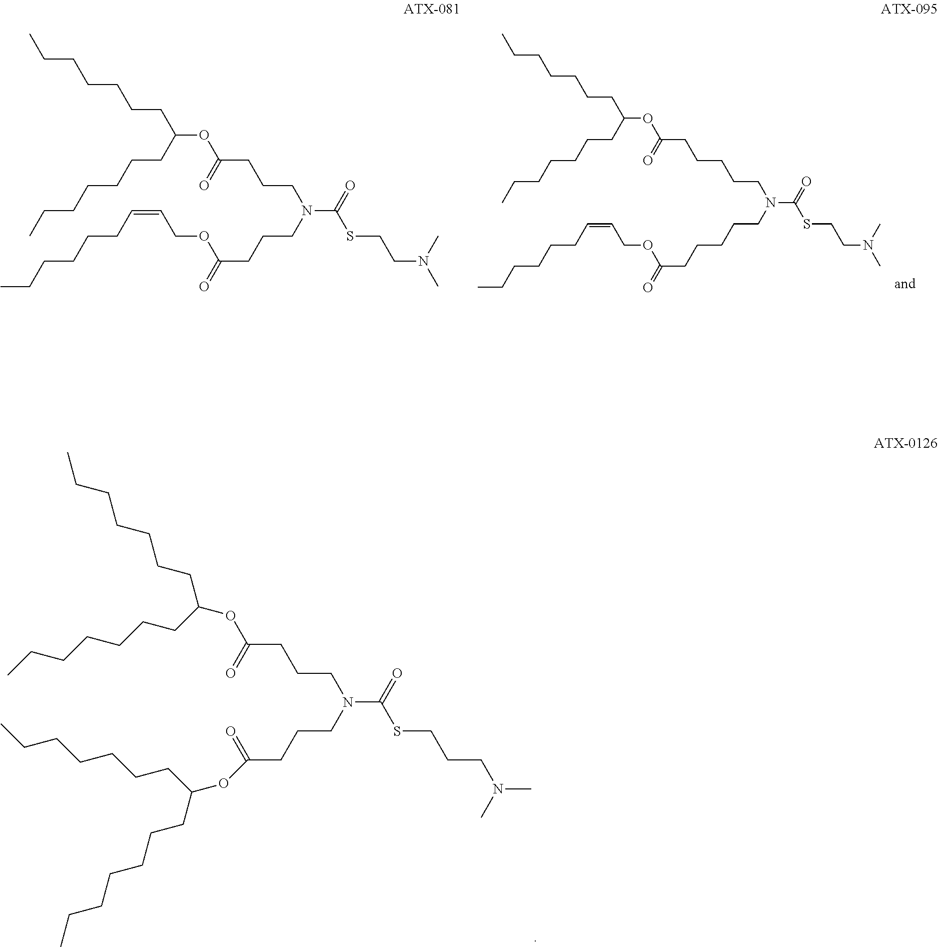

In one aspect, the ionizable cationic lipid of compositions provided herein has a structure of

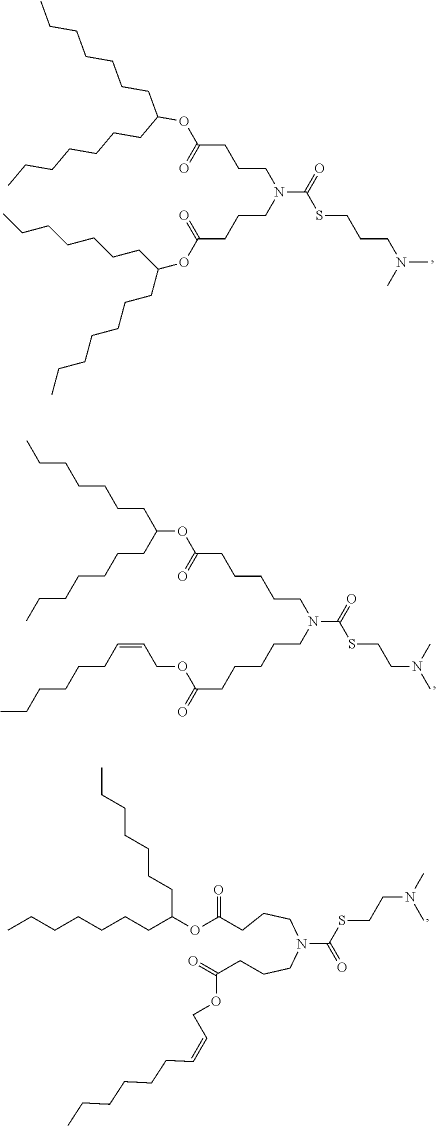

or a pharmaceutically acceptable salt thereof.

In another aspect, the ionizable cationic lipid of compositions provided herein has a structure of

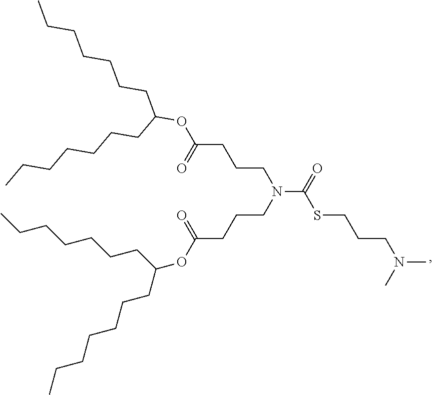 [SMILES: CCCCCCCC(CCCCCCC)OC(=O)CCCN(CCCC(=O)OC(CCCCCCC)CCCCCCC)C(=O)SCCCN(C)C]

or a pharmaceutically acceptable salt thereof.

In one aspect, the ionizable cationic lipid included in lipid formulations of pharmaceutical compositions provided herein has a structure of

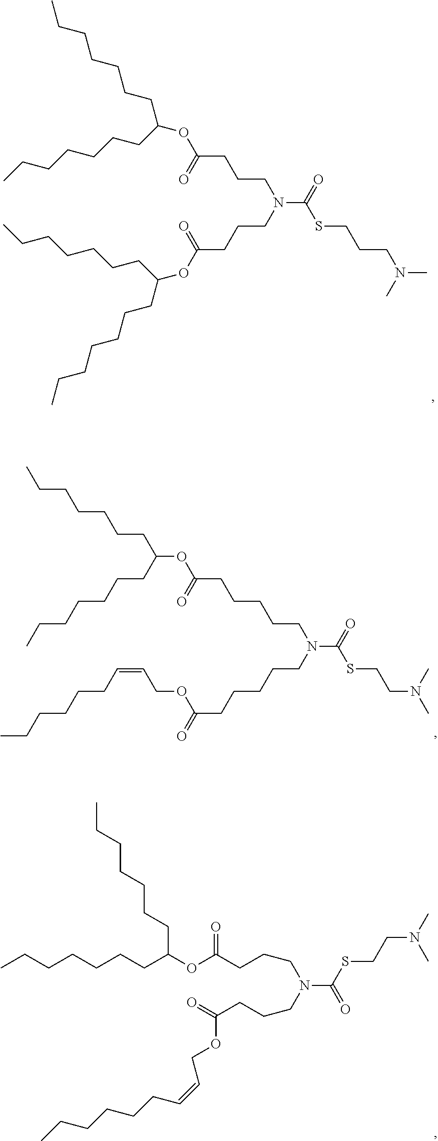

or a pharmaceutically acceptable salt thereof.

In another aspect, the ionizable cationic lipid included in lipid formulations of pharmaceutical compositions provided herein has a structure of

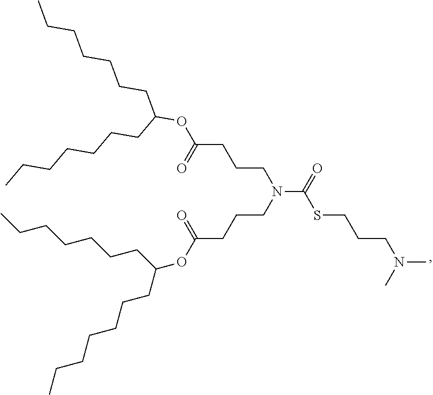 [SMILES: CCCCCCCC(CCCCCCC)OC(=O)CCCN(CCCC(=O)OC(CCCCCCC)CCCCCCC)C(=O)SCCCN(C)C]

or a pharmaceutically acceptable salt thereof.

### Lipid Formulations/LNPs

Therapies based on the intracellular delivery of nucleic acids to target cells face both extracellular and intracellular barriers. Indeed, naked nucleic acid materials cannot be easily systemically administered due to their toxicity, low stability in serum, rapid renal clearance, reduced uptake by target cells, phagocyte uptake and their ability in activating the immune response, all features that preclude their clinical development. When exogenous nucleic acid material (e.g., mRNA) enters the human biological system, it is recognized by the reticuloendothelial system (RES) as foreign pathogens and cleared from blood circulation before having the chance to encounter target cells within or outside the vascular system. It has been reported that the half-life of naked nucleic acid in the blood stream is around several minutes (Kawabata K, Takakura Y, Hashida MPharm Res. 1995 June; 12(6):825-30). Chemical modification and a proper delivery method can reduce uptake by the RES and protect nucleic acids from degradation by ubiquitous nucleases, which increase stability and efficacy of nucleic acid-based therapies. In addition, RNAs or DNAs are anionic hydrophilic polymers that are not favorable for uptake by cells, which are also anionic at the surface. The success of nucleic acid-based therapies thus depends largely on the development of vehicles or vectors that can efficiently and effectively deliver genetic material to target cells and obtain sufficient levels of expression in vivo with minimal toxicity.

Moreover, upon internalization into a target cell, nucleic acid delivery vectors are challenged by intracellular barriers, including endosome entrapment, lysosomal degradation, nucleic acid unpacking from vectors, translocation across the nuclear membrane (for DNA), release at the cytoplasm (for RNA), and so on. Successful nucleic acid-based therapy thus depends upon the ability of the vector to deliver the nucleic acids to the target sites inside of the cells in order to obtain sufficient levels of a desired activity such as expression of a gene.

While several gene therapies have been able to successfully utilize a viral delivery vector (e.g., AAV), lipid-based formulations have been increasingly recognized as one of the most promising delivery systems for RNA and other nucleic acid compounds due to their biocompatibility and their ease of large-scale production. One of the most significant advances in lipid-based nucleic acid therapies happened in August 2018 when Patisiran (ALN-TTR02) was the first siRNA therapeutic approved by the Food and Drug Administration (FDA) and by the European Commission (EC). ALN-TTR02 is an siRNA formulation based upon the so-called Stable Nucleic Acid Lipid Particle (SNALP) transfecting technology. Despite the success of Patisiran, the delivery of nucleic acid therapeutics, including mRNA, via lipid formulations is still under ongoing development.

Some art-recognized lipid-formulated delivery vehicles for nucleic acid therapeutics include, according to various embodiments, polymer based carriers, such as polyethyleneimine (PEI), lipid nanoparticles and liposomes, nanoliposomes, ceramide-containing nanoliposomes, multivesicular liposomes, proteoliposomes, both natural and synthetically-derived exosomes, natural, synthetic and semi-synthetic lamellar bodies, nanoparticulates, micelles, and emulsions. These lipid formulations can vary in their structure and composition, and as can be expected in a rapidly evolving field, several different terms have been used in the art to describe a single type of delivery vehicle. At the same time, the terms for lipid formulations have varied as to their intended meaning throughout the scientific literature, and this inconsistent use has caused confusion as to the exact meaning of several terms for lipid formulations. Among the several potential lipid formulations, liposomes, cationic liposomes, and lipid nanoparticles are specifically described in detail and defined herein for the purposes of the present disclosure.

### Liposomes

Conventional liposomes are vesicles that consist of at least one bilayer and an internal aqueous compartment. Bilayer membranes of liposomes are typically formed by amphiphilic molecules, such as lipids of synthetic or natural origin that comprise spatially separated hydrophilic and hydrophobic domains (Lasic, Trends Biotechnol., 16: 307-321, 1998). Bilayer membranes of the liposomes can also be formed by amphiphilic polymers and surfactants (e.g., polymerosomes, niosomes, etc.). They generally present as spherical vesicles and can range in size from 20 nm to a few microns. Liposomal formulations can be prepared as a colloidal dispersion or they can be lyophilized to reduce stability risks and to improve the shelf-life for liposome-based drugs. Methods of preparing liposomal compositions are known in the art and would be within the skill of an ordinary artisan.

Liposomes that have only one bilayer are referred to as being unilamellar, and those having more than one bilayer are referred to as multilamellar. The most common types of liposomes are small unilamellar vesicles (SUV), large unilamellar vesicle (LUV), and multilamellar vesicles (MLV). In contrast to liposomes, lysosomes, micelles, and reversed micelles are composed of monolayers of lipids. Generally, a liposome is thought of as having a single interior compartment, however some formulations can be multivesicular liposomes (MVL), which consist of numerous discontinuous internal aqueous compartments separated by several nonconcentric lipid bilayers.

Liposomes have long been perceived as drug delivery vehicles because of their superior biocompatibility, given that liposomes are basically analogs of biological membranes, and can be prepared from both natural and synthetic phospholipids (Int J Nanomedicine. 2014; 9:1833-1843). In their use as drug delivery vehicles, because a liposome has an aqueous solution core surrounded by a hydrophobic membrane, hydrophilic solutes dissolved in the core cannot readily pass through the bilayer, and hydrophobic compounds will associate with the bilayer. Thus, a liposome can be loaded with hydrophobic and/or hydrophilic molecules. When a liposome is used to carry a nucleic acid such as RNA, the nucleic acid will be contained within the liposomal compartment in an aqueous phase.

### Cationic Liposomes

Liposomes can be composed of cationic, anionic, and/or neutral lipids. As an important subclass of liposomes, cationic liposomes are liposomes that are made in whole or part from positively charged lipids, or more specifically a lipid that comprises both a cationic group and a lipophilic portion. In addition to the general characteristics profiled above for liposomes, the positively charged moieties of cationic lipids used in cationic liposomes provide several advantages and some unique structural features. For example, the lipophilic portion of the cationic lipid is hydrophobic and thus will direct itself away from the aqueous interior of the liposome and associate with other nonpolar and hydrophobic species. Conversely, the cationic moiety will associate with aqueous media and more importantly with polar molecules and species with which it can complex in the aqueous interior of the cationic liposome. For these reasons, cationic liposomes are increasingly being researched for use in gene therapy due to their favorability towards negatively charged nucleic acids via electrostatic interactions, resulting in complexes that offer biocompatibility, low toxicity, and the possibility of the large-scale production required for in vivo clinical applications. Cationic lipids suitable for use in cationic liposomes are listed herein below.

### Lipid Nanoparticles

In contrast to liposomes and cationic liposomes, lipid nanoparticles (LNP) have a structure that includes a single monolayer or bilayer of lipids that encapsulates a compound in a solid phase. Thus, unlike liposomes, lipid nanoparticles do not have an aqueous phase or other liquid phase in its interior, but rather the lipids from the bilayer or monolayer shell are directly complexed to the internal compound thereby encapsulating it in a solid core. Lipid nanoparticles are typically spherical vesicles having a relatively uniform dispersion of shape and size. While sources vary on what size qualifies a lipid particle as being a nanoparticle, there is some overlap in agreement that a lipid nanoparticle can have a diameter in the range of from 10 nm to 1000 nm. However, more commonly they are considered to be smaller than 120 nm or even 100 nm.

For lipid nanoparticle nucleic acid delivery systems, the lipid shell is formulated to include an ionizable cationic lipid which can complex to and associate with the negatively charged backbone of the nucleic acid core. Ionizable cationic lipids with apparent pKa values below about 7 have the benefit of providing a cationic lipid for complexing with the nucleic acid's negatively charged backbone and loading into the lipid nanoparticle at pH values below the pKa of the ionizable lipid where it is positively charged. Then, at physiological pH values, the lipid nanoparticle can adopt a relatively neutral exterior allowing for a significant increase in the circulation half-lives of the particles following i.v. administration. In the context of nucleic acid delivery, lipid nanoparticles offer many advantages over other lipid-based nucleic acid delivery systems including high nucleic acid encapsulation efficiency, potent transfection, improved penetration into tissues to deliver therapeutics, and low levels of cytotoxicity and immunogenicity.

Prior to the development of lipid nanoparticle delivery systems for nucleic acids, cationic lipids were widely studied as synthetic materials for delivery of nucleic acid medicines. In these early efforts, after mixing together at physiological pH, nucleic acids were condensed by cationic lipids to form lipid-nucleic acid complexes known as lipoplexes. However, lipoplexes proved to be unstable and characterized by broad size distributions ranging from the submicron scale to a few microns. Lipoplexes, such as the Lipofectamine® reagent, have found considerable utility for in vitro transfection. However, these first-generation lipoplexes have not proven useful in vivo. The large particle size and positive charge (Imparted by the cationic lipid) result in rapid plasma clearance, hemolytic and other toxicities, as well as immune system activation. In some aspects, nucleic acid molecules provided herein and lipids or lipid formulations provided herein form a lipid nanoparticle (LNP).

In other aspects, nucleic acid molecules provided herein are incorporated into a lipid formulation (i.e., a lipid-based delivery vehicle).

In the context of the present disclosure, a lipid-based delivery vehicle typically serves to transport a desired RNA to a target cell or tissue. The lipid-based delivery vehicle can be any suitable lipid-based delivery vehicle known in the art. In some aspects, the lipid-based delivery vehicle is a liposome, a cationic liposome, or a lipid nanoparticle containing a self-replicating RNA of the disclosure. In some aspects, the lipid-based delivery vehicle comprises a nanoparticle or a bilayer of lipid molecules and a self-replicating RNA of the disclosure. In some aspects, the lipid bilayer further comprises a neutral lipid or a polymer. In some aspects, the lipid formulation comprises a liquid medium. In some aspects, the formulation further encapsulates a nucleic acid. In some aspects, the lipid formulation further comprises a nucleic acid and a neutral lipid or a polymer. In some aspects, the lipid formulation encapsulates the nucleic acid.

The description provides lipid formulations comprising one or more self-replicating RNA molecules encapsulated within the lipid formulation. In some aspects, the lipid formulation comprises liposomes. In some aspects, the lipid formulation comprises cationic liposomes. In some aspects, the lipid formulation comprises lipid nanoparticles.

In some aspects, the self-replicating RNA is fully encapsulated within the lipid portion of the lipid formulation such that the RNA in the lipid formulation is resistant in aqueous solution to nuclease degradation. In other aspects, the lipid formulations described herein are substantially non-toxic to animals such as humans and other mammals.

The lipid formulations of the disclosure also typically have a total lipid:RNA ratio (mass/mass ratio) of from about 1:1 to about 100:1, from about 1:1 to about 50:1, from about 2:1 to about 45:1, from about 3:1 to about 40:1, from about 5:1 to about 45:1, or from about 10:1 to about 40:1, or from about 15:1 to about 40:1, or from about 20:1 to about 40:1; or from about 25:1 to about 45:1; or from about 30:1 to about 45:1; or from about 32:1 to about 42:1; or from about 34:1 to about 42:1. In some aspects, the total lipid:RNA ratio (mass/mass ratio) is from about 30:1 to about 45:1. The ratio may be any value or subvalue within the recited ranges, including endpoints.

The lipid formulations of the present disclosure typically have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, or about 150 nm, and are substantially non-toxic. The diameter may be any value or subvalue within the recited ranges, including endpoints. In addition, nucleic acids, when present in the lipid nanoparticles of the present disclosure, generally are resistant in aqueous solution to degradation with a nuclease.

In some embodiments, the lipid nanoparticle has a size of less than about 500 nm, less than about 400 nm, less than about 300 nm, less than about 200 nm, less than about 100 nm, or less than about 50 nm. In specific embodiments, the lipid nanoparticle has a size of about 55 nm to about 90 nm.

In some aspects, the lipid formulations comprise a self-replicating RNA, a cationic lipid (e.g., one or more cationic lipids or salts thereof described herein), a phospholipid, and a conjugated lipid that inhibits aggregation of the particles (e.g., one or more PEG-lipid conjugates). The lipid formulations can also include cholesterol. In one aspect, the cationic lipid is an ionizable cationic lipid.

In the nucleic acid-lipid formulations, the RNA may be fully encapsulated within the lipid portion of the formulation, thereby protecting the nucleic acid from nuclease degradation. In some aspects, a lipid formulation comprising an RNA is fully encapsulated within the lipid portion of the lipid formulation, thereby protecting the nucleic acid from nuclease degradation. In certain aspects, the RNA in the lipid formulation is not substantially degraded after exposure of the particle to a nuclease at 37° C. for at least 20, 30, 45, or 60 minutes. In certain other aspects, the RNA in the lipid formulation is not substantially degraded after incubation of the formulation in serum at 37° C. for at least 30, 45, or 60 minutes or at least 2, 3, 4, 5, 6, 7, 8, 9, 10, 12, 14, 16, 18, 20, 22, 24, 26, 28, 30, 32, 34, or 36 hours. In some aspects, the RNA is complexed with the lipid portion of the formulation. One of the benefits of the formulations of the present disclosure is that the nucleic acid-lipid compositions are substantially non-toxic to animals such as humans and other mammals.

In the context of nucleic acids, full encapsulation may be determined by performing a membrane-impermeable fluorescent dye exclusion assay, which uses a dye that has enhanced fluorescence when associated with nucleic acid. Encapsulation is determined by adding the dye to a lipid formulation, measuring the resulting fluorescence, and comparing it to the fluorescence observed upon addition of a small amount of nonionic detergent. Detergent-mediated disruption of the lipid layer releases the encapsulated nucleic acid, allowing it to interact with the membrane-impermeable dye. Nucleic acid encapsulation may be calculated as E=(I0−I)/I0, where/and I0 refers to the fluorescence intensities before and after the addition of detergent.

In some aspects, the present disclosure provides a nucleic acid-lipid composition comprising a plurality of nucleic acid-liposomes, nucleic acid-cationic liposomes, or nucleic acid-lipid nanoparticles. In some aspects, the nucleic acid-lipid composition comprises a plurality of RNA-liposomes. In some aspects, the nucleic acid-lipid composition comprises a plurality of RNA-cationic liposomes. In some aspects, the nucleic acid-lipid composition comprises a plurality of RNA-lipid nanoparticles.

In some aspects, the lipid formulations comprise RNA that is fully encapsulated within the lipid portion of the formulation, such that from about 30% to about 100%, from about 40% to about 100%, from about 50% to about 100%, from about 60% to about 100%, from about 70% to about 100%, from about 80% to about 100%, from about 90% to about 100%, from about 30% to about 95%, from about 40% to about 95%, from about 50% to about 95%, from about 60% to about 95%, from about 70% to about 95%, from about 80% to about 95%, from about 85% to about 95%, from about 90% to about 95%, from about 30% to about 90%, from about 40% to about 90%, from about 50% to about 90%, from about 60% to about 90%, from about 70% to about 90%, from about 80% to about 90%, or at least about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99% (or any fraction thereof or range therein) of the particles have the RNA encapsulated therein. The amount may be any value or subvalue within the recited ranges, including endpoints. The RNA included in any RNA-lipid composition or RNA-lipid formulation provided herein can be a self-replicating RNA.

Depending on the intended use of the lipid formulation, the proportions of the components can be varied, and the delivery efficiency of a particular formulation can be measured using assays known in the art.

In some aspects, nucleic acid molecules provided herein are lipid formulated. The lipid formulation is preferably selected from, but not limited to, liposomes, cationic liposomes, and lipid nanoparticles. In one aspect, a lipid formulation is a cationic liposome or a lipid nanoparticle (LNP) comprising:
(a) an RNA of the present disclosure,
(b) a cationic lipid,
(c) an aggregation reducing agent (such as polyethylene glycol (PEG) lipid or PEG-modified lipid),
(d) optionally a non-cationic lipid (such as a neutral lipid), and
(e) optionally, a sterol.

In another aspect, the cationic lipid is an ionizable cationic lipid. Any ionizable cationic lipid can be included in lipid formulations, including exemplary cationic lipids provided herein.

### Cationic Lipids

In one aspect, the lipid nanoparticle formulation comprises (i) at least one cationic lipid; (ii) a helper lipid; (iii) a sterol (e.g., cholesterol); and (iv) a PEG-lipid. In another aspect, the cationic lipid is an ionizable cationic lipid. In yet another aspect, the lipid nanoparticle formulation comprises (i) at least one cationic lipid; (ii) a helper lipid; (iii) a sterol (e.g., cholesterol); and (iv) a PEG-lipid, in a molar ratio of about 40-70% ionizable cationic lipid: about 2-15% helper lipid: about 20-45% sterol; about 0.5-5% PEG-lipid. In a further aspect, the cationic lipid is an ionizable cationic lipid.

In one aspect, the lipid nanoparticle formulation consists of (i) at least one cationic lipid; (ii) a helper lipid; (iii) a sterol (e.g., cholesterol); and (iv) a PEG-lipid. In another aspect, the cationic lipid is an ionizable cationic lipid. In yet another aspect, the lipid nanoparticle formulation consists of (i) at least one cationic lipid; (ii) a helper lipid; (iii) a sterol (e.g., cholesterol); and (iv) a PEG-lipid, in a molar ratio of about 40-70% ionizable cationic lipid: about 2-15% helper lipid: about 20-45% sterol; about 0.5-5% PEG-lipid. In a further aspect, the cationic lipid is an ionizable cationic lipid.

In the presently disclosed lipid formulations, the cationic lipid may be, for example, N,N-dioleyl-N,N-dimethylammonium chloride (DODAC), N,N-distearyl-N,N-dimethylammonium bromide (DDAB), 1,2-dioleoyltrimethylammoniumpropane chloride (DOTAP) (also known as N-(2,3-dioleoyloxy)propyl)-N,N,N-trimethylammonium chloride and 1,2-Dioleyloxy-3-trimethylaminopropane chloride salt), N-(1-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-di-y-linolenyloxy-N,N-dimethylaminopropane (γ-DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanediol (DOAP), 1,2-Dilinoleyloxo-3-(2-N,N-dimethylamino)ethoxypropane (DLin-EG-DMA), 2,2-Dilinoleyl-4-dimethylaminomethyl-[1,3]-dioxolane (DLin-K-DMA) or analogs thereof, (3aR,5s,6aS)—N,N-dimethyl-2,2-di((9Z,12Z)-octadeca-9,12-dienyl)tetrahydro-3aH-cyclopenta[d][1,3]dioxol-5-amine, (6Z,9Z,28Z,31Z)-heptatriaconta-6,9,28,31-tetraen-19-yl4-(dimethylamino)butanoate (MC3), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (C12-200), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DLin-K-DMA), (6Z,9Z,28Z,31Z)-heptatriaconta-6,9,28 31-tetraen-19-yl 4-(dimethylamino) butanoate (DLin-M-C3-DMA), 3-((6Z,9Z,28Z,31Z)-heptatriaconta-6,9,28,31-tetraen-19-yloxy)-N,N-dimethylpropan-1-amine (MC3 Ether), 4-((6Z,9Z,28Z,31 Z)-heptatriaconta-6,9,28,31-tetraen-19-yloxy)-N,N-dimethylbutan-1-amine (MC4 Ether), or any combination thereof. Other cationic lipids include, but are not limited to, N,N-distearyl-N,N-dimethylammonium bromide (DDAB), 3P—(N—(N′,N′-dimethylaminoethane)-carbamoyl)cholesterol (DC-Choi), N-(1-(2,3-dioleyloxy)propyl)-N-2-(sperminecarboxamido)ethyl)-N,N-dimethylammonium trifluoracetate (DOSPA), dioctadecylamidoglycyl carboxyspermine (DOGS), 1,2-dileoyl-sn-3-phosphoethanolamine (DOPE), 1,2-dioleoyl-3-dimethylammonium propane (DODAP), N-(1,2-dimyristyloxyprop-3-yl)-N,N-dimethyl-N-hydroxyethyl ammonium bromide (DMRIE), and 2,2-Dilinoleyl-4-dimethylaminoethyl-[1,3]-dioxolane (XTC). Additionally, commercial preparations of cationic lipids can be used, such as, e.g., LIPOFECTIN (including DOTMA and DOPE, available from GIBCO/BRL), and Lipofectamine (comprising DOSPA and DOPE, available from GIBCO/BRL).

Other suitable cationic lipids are disclosed in International Publication Nos. WO 09/086558, WO 09/127060, WO 10/048536, WO 10/054406, WO 10/088537, WO 10/129709, and WO 2011/153493; U.S. Patent Publication Nos. 2011/0256175, 2012/0128760, and 2012/0027803; U.S. Pat. Nos. 8,158,601; and Love et al., PNAS, 107(5), 1864-69, 2010, the contents of which are herein incorporated by reference.

The RNA-lipid formulations of the present disclosure can comprise a helper lipid, which can be referred to as a neutral helper lipid, non-cationic lipid, non-cationic helper lipid, anionic lipid, anionic helper lipid, or a neutral lipid. It has been found that lipid formulations, particularly cationic liposomes and lipid nanoparticles have increased cellular uptake if helper lipids are present in the formulation. (Curr. Drug Metab. 2014; 15(9):882-92). For example, some studies have indicated that neutral and zwitterionic lipids such as 1,2-dioleoylsn-glycero-3-phosphatidylcholine (DOPC), Di-Oleoyl-Phosphatidyl-Ethanoalamine (DOPE) and 1,2-DiStearoyl-sn-glycero-3-PhosphoCholine (DSPC), being more fusogenic (i.e., facilitating fusion) than cationic lipids, can affect the polymorphic features of lipid-nucleic acid complexes, promoting the transition from a lamellar to a hexagonal phase, and thus inducing fusion and a disruption of the cellular membrane. (Nanomedicine (Lond). 2014 January; 9(1):105-20). In addition, the use of helper lipids can help to reduce any potential detrimental effects from using many prevalent cationic lipids such as toxicity and immunogenicity.

Non-limiting examples of non-cationic lipids suitable for lipid formulations of the present disclosure include phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, dilinoleoylphosphatidylcholine, and mixtures thereof. Other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, palmitoyl, stearoyl, or oleoyl.

Additional examples of non-cationic lipids include sterols such as cholesterol and derivatives thereof. As a helper lipid, cholesterol increases the spacing of the charges of the lipid layer interfacing with the nucleic acid making the charge distribution match that of the nucleic acid more closely. (J. R. Soc. Interface. 2012 Mar. 7; 9(68): 548-561). Non-limiting examples of cholesterol derivatives include polar analogues such as 5α-cholestanol, 5α-coprostanol, cholesteryl-(2′-hydroxy)-ethyl ether, cholesteryl-(4′-hydroxy)-butyl ether, and 6-ketocholestanol; non-polar analogues such as 5α-cholestane, cholestenone, 5α-cholestanone, 5α-cholestanone, and cholesteryl decanoate; and mixtures thereof. In some aspects, the cholesterol derivative is a polar analogue such as cholesteryl-(4′-hydroxy)-butyl ether.

In some aspects, the helper lipid present in the lipid formulation comprises or consists of a mixture of one or more phospholipids and cholesterol or a derivative thereof. In other aspects, the neutral lipid present in the lipid formulation comprises or consists of one or more phospholipids, e.g., a cholesterol-free lipid formulation. In yet other aspects, the neutral lipid present in the lipid formulation comprises or consists of cholesterol or a derivative thereof, e.g., a phospholipid-free lipid formulation.

Other examples of helper lipids include nonphosphorous containing lipids such as, e.g., stearylamine, dodecylamine, hexadecylamine, acetyl palmitate, glycerol ricinoleate, hexadecyl stearate, isopropyl myristate, amphoteric acrylic polymers, triethanolamine-lauryl sulfate, alkyl-aryl sulfate polyethyloxylated fatty acid amides, dioctadecyldimethyl ammonium bromide, ceramide, and sphingomyelin.

Other suitable cationic lipids include those having alternative fatty acid groups and other dialkylamino groups, including those, in which the alkyl substituents are different (e.g., N-ethyl-N-methylamino-, and N-propyl-N-ethylamino-). These lipids are part of a subcategory of cationic lipids referred to as amino lipids. In some embodiments of the lipid formulations described herein, the cationic lipid is an amino lipid. In general, amino lipids having less saturated acyl chains are more easily sized, particularly when the complexes must be sized below about 0.3 microns, for purposes of filter sterilization. Amino lipids containing unsaturated fatty acids with carbon chain lengths in the range of C14 to C22 may be used. Other scaffolds can also be used to separate the amino group and the fatty acid or fatty alkyl portion of the amino lipid.

In some embodiments, the lipid formulation comprises the cationic lipid with Formula I according to the patent application PCT/EP2017/064066. In this context, the disclosure of PCT/EP2017/064066 is also incorporated herein by reference.

In some embodiments, amino or cationic lipids of the present disclosure are ionizable and have at least one protonatable or deprotonatable group, such that the lipid is positively charged at a pH at or below physiological pH (e.g., pH 7.4), and neutral at a second pH, preferably at or above physiological pH. Of course, it will be understood that the addition or removal of protons as a function of pH is an equilibrium process, and that the reference to a charged or a neutral lipid refers to the nature of the predominant species and does not require that all of the lipid be present in the charged or neutral form. Lipids that have more than one protonatable or deprotonatable group, or which are zwitterionic, are not excluded from use in the disclosure. In certain embodiments, the protonatable lipids have a pKa of the protonatable group in the range of about 4 to about 11. In some embodiments, the ionizable cationic lipid has a pKa of about 5 to about 7. In some embodiments, the pKa of an ionizable cationic lipid is about 6 to about 7.

In some embodiments, the lipid formulation comprises an ionizable cationic lipid of Formula I:

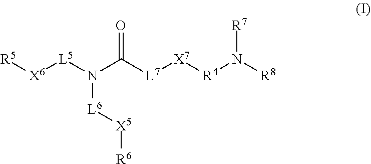

or a pharmaceutically acceptable salt or solvate thereof, wherein R5 and R6 are each independently selected from the group consisting of a linear or branched C1-C31 alkyl, C2-C31 alkenyl or C2-C31 alkynyl and cholesteryl; L5 and L6 are each independently selected from the group consisting of a linear C1-C20 alkyl and C2-C20 alkenyl; X5 is —C(O)O—, whereby —C(O)O—R6 is formed or —OC(O)— whereby —OC(O)—R6 is formed; X6 is —C(O)O— whereby —C(O)O—R5 is formed or —OC(O)— whereby —OC(O)—R5 is formed; X7 is S or O; L7 is absent or lower alkyl; R4 is a linear or branched C1-C6 alkyl; and R7 and R8 are each independently selected from the group consisting of a hydrogen and a linear or branched C1-C6 alkyl.

In some embodiments, X7 is S.

In some embodiments, X5 is —C(O)O—, whereby —C(O)O—R6 is formed and X6 is —C(O)O— whereby —C(O)O—R5 is formed.

In some embodiments, R7 and R8 are each independently selected from the group consisting of methyl, ethyl and isopropyl.

In some embodiments, L5 and L6 are each independently a C1-C10 alkyl. In some embodiments, L5 is C1-C3 alkyl, and L6 is C1-C5 alkyl. In some embodiments, L6 is C1-C2 alkyl. In some embodiments, L5 and L6 are each a linear C7 alkyl. In some embodiments, L5 and L6 are each a linear C9 alkyl.

In some embodiments, R5 and R6 are each independently an alkenyl. In some embodiments, R6 is alkenyl. In some embodiments, R6 is C2-C9 alkenyl. In some embodiments, the alkenyl comprises a single double bond. In some embodiments, R5 and R6 are each alkyl. In some embodiments, R5 is a branched alkyl. In some embodiments, R5 and R6 are each independently selected from the group consisting of a C9 alkyl, C9 alkenyl and C9 alkynyl. In some embodiments, R5 and R6 are each independently selected from the group consisting of a C11 alkyl, C11 alkenyl and C11 alkynyl. In some embodiments, R5 and R6 are each independently selected from the group consisting of a C7 alkyl, C7 alkenyl and C7 alkynyl. In some embodiments, R5 is —CH((CH2)pCH3)2 or —CH((CH2)pCH3)((CH2)p-1CH3), wherein p is 4-8. In some embodiments, p is 5 and L5 is a C1-C3 alkyl. In some embodiments, p is 6 and L5 is a C3 alkyl. In some embodiments, p is 7. In some embodiments, p is 8 and L5 is a C1-C3 alkyl. In some embodiments, R5 consists of —CH((CH2)pCH3)((CH2)p-1CH3), wherein p is 7 or 8.

In some embodiments, R4 is ethylene or propylene. In some embodiments, R4 is n-propylene or isobutylene.

In some embodiments, L7 is absent, R4 is ethylene, X7 is S and R7 and R8 are each methyl. In some embodiments, L7 is absent, R4 is n-propylene, X7 is S and R7 and R8 are each methyl. In some embodiments, L7 is absent, R4 is ethylene, X7 is S and R7 and R8 are each ethyl.

In some embodiments, X7 is S, X5 is —C(O)O—, whereby —C(O)O—R6 is formed, X6 is —C(O)O— whereby —C(O)O—R5 is formed, L5 and L6 are each independently a linear C3-C7 alkyl, L7 is absent, R5 is —CH((CH2)pCH3)2, and R6 is C7-C12 alkenyl. In some further embodiments, p is 6 and R6 is C9 alkenyl.

In some embodiments, the lipid formulation can comprise an ionizable cationic lipid selected from the group consisting of LIPID #1 to LIPID #8:

TABLE 6

| LIPID # | STRUCTURE |
|---|---|
| 1 | 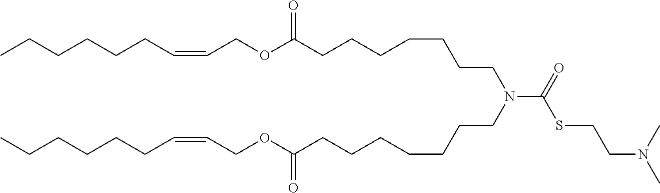 [SMILES: CCCCCC/C=C\COC(=O)CCCCCCCN(CCCCCCCC(=O)OC/C=C\CCCCCC)C(=O)SCCN(C)C] |
| 2 | 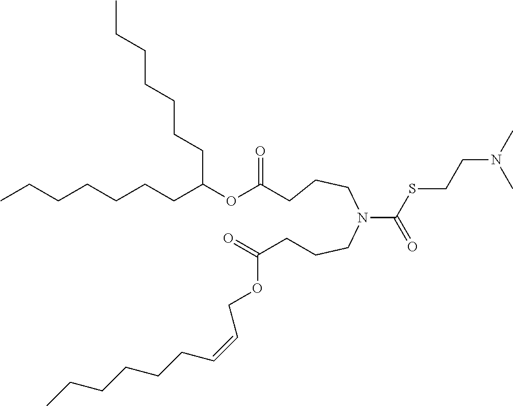 [SMILES: CCCCCC/C=C\COC(=O)CCCN(CCCC(=O)OC(CCCCCCC)CCCCCCC)C(=O)SCCN(C)C] |
| 3 | 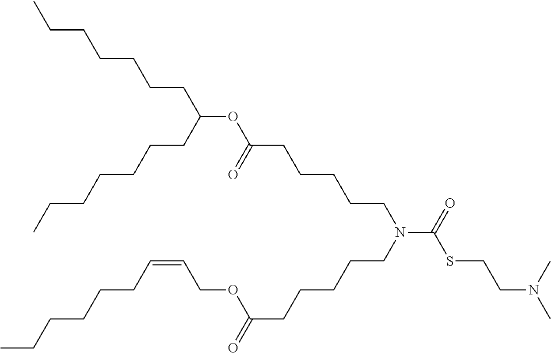 [SMILES: CCCCCC/C=C\COC(=O)CCCCCN(CCCCCC(=O)OC(CCCCCCC)CCCCCCC)C(=O)SCCN(C)C] |
| 4 | 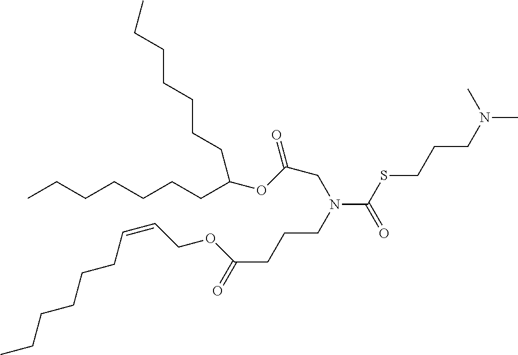 [SMILES: CCCCCC/C=C\COC(=O)CCCN(CC(=O)OC(CCCCCCC)CCCCCCC)C(=O)SCCCN(C)C] |
| 5 | 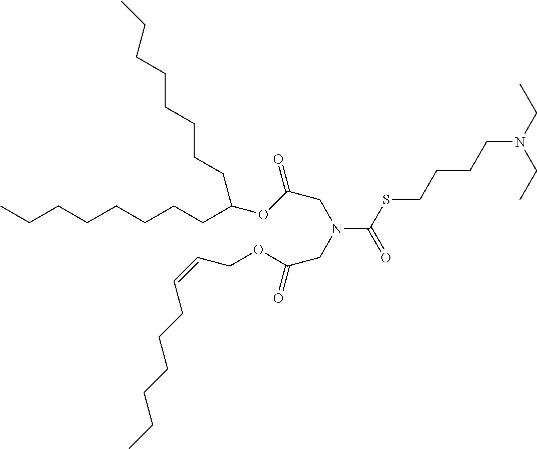 [SMILES: CCCCCC/C=C\COC(=O)CN(CC(=O)OC(CCCCCCCC)CCCCCCCC)C(=O)SCCCCN(CC)CC] |
| 6 | 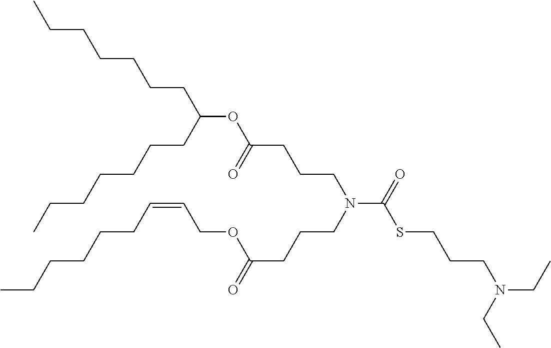 [SMILES: CCCCCC/C=C\COC(=O)CCCN(CCCC(=O)OC(CCCCCCC)CCCCCCC)C(=O)SCCCN(CC)CC] |
| 7 | 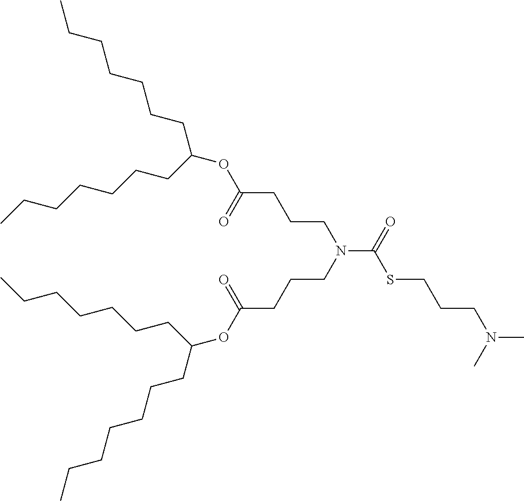 [SMILES: CCCCCCCC(CCCCCCC)OC(=O)CCCN(CCCC(=O)OC(CCCCCCC)CCCCCCC)C(=O)SCCCN(C)C] |
| 8 | 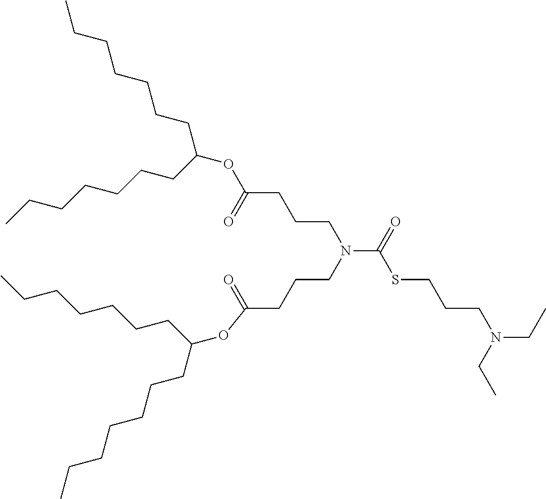 [SMILES: CCCCCCCC(CCCCCCC)OC(=O)CCCN(CCCC(=O)OC(CCCCCCC)CCCCCCC)C(=O)SCCCN(CC)CC] |

In some embodiments, the lipid formulation comprises an ionizable cationic lipid having a structure selected from

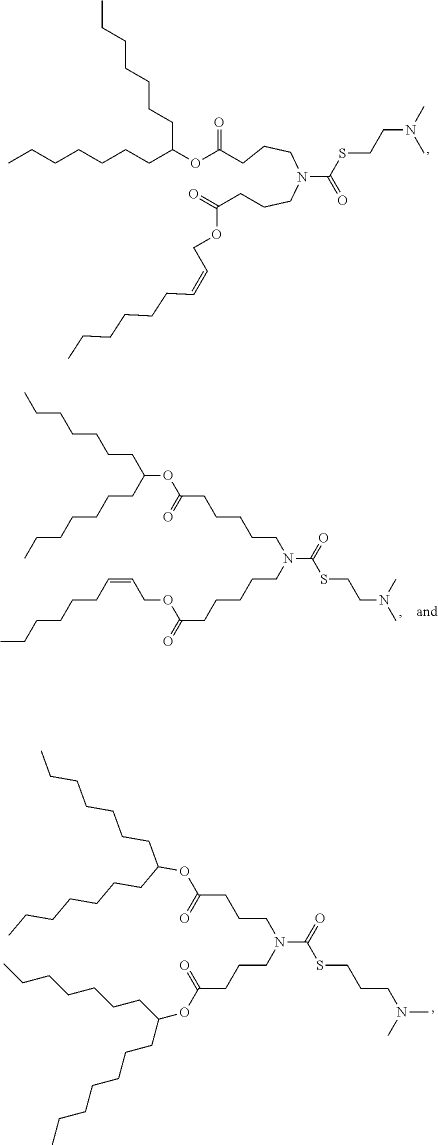

or a pharmaceutically acceptable salt thereof.

In some preferred embodiments, the lipid formulation comprises an ionizable cationic lipid having the structure

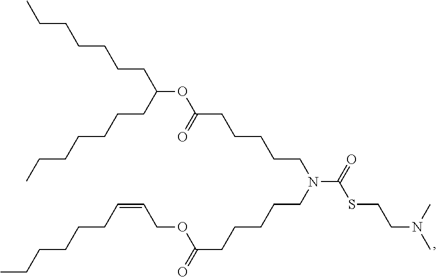 [SMILES: CCCCCC/C=C\COC(=O)CCCCCN(CCCCCC(=O)OC(CCCCCCC)CCCCCCC)C(=O)SCCN(C)C]

or a pharmaceutically acceptable salt thereof.

In embodiments, any one or more lipids recited herein may be expressly excluded.

In some aspects, the helper lipid comprises from about 2 mol % to about 20 mol %, from about 3 mol % to about 18 mol %, from about 4 mol % to about 16 mol %, about 5 mol % to about 14 mol %, from about 6 mol % to about 12 mol %, from about 5 mol % to about 10 mol %, from about 5 mol % to about 9 mol %, or about 2 mol %, about 3 mol %, about 4 mol %, about 5 mol %, about 6 mol %, about 7 mol %, about 8 mol %, about 9 mol %, about 10 mol %, about 11 mol %, or about 12 mol % (or any fraction thereof or the range therein) of the total lipid present in the lipid formulation.

The lipid portion, or the cholesterol or cholesterol derivative in the lipid formulation may comprise up to about 40 mol %, about 45 mol %, about 50 mol %, about 55 mol %, or about 60 mol % of the total lipid present in the lipid formulation. In some aspects, the cholesterol or cholesterol derivative comprises about 15 mol % to about 45 mol %, about 20 mol % to about 40 mol %, about 25 mol % to about 35 mol %, or about 28 mol % to about 35 mol %; or about 25 mol %, about 26 mol %, about 27 mol %, about 28 mol %, about 29 mol %, about 30 mol %, about 31 mol %, about 32 mol %, about 33 mol %, about 34 mol %, about 35 mol %, about 36 mol %, or about 37 mol % of the total lipid present in the lipid formulation.

In specific embodiments, the lipid portion of the lipid formulation is about 35 mol % to about 42 mol % cholesterol.

In some aspects, the phospholipid component in the mixture may comprise from about 2 mol % to about 20 mol %, from about 3 mol % to about 18 mol %, from about 4 mol % to about 16 mol %, about 5 mol % to about 14 mol %, from about 6 mol % to about 12 mol %, from about 5 mol % to about 10 mol %, from about 5 mol % to about 9 mol %, or about 2 mol %, about 3 mol %, about 4 mol %, about 5 mol %, about 6 mol %, about 7 mol %, about 8 mol %, about 9 mol %, about 10 mol %, about 11 mol %, or about 12 mol % (or any fraction thereof or the range therein) of the total lipid present in the lipid formulation.

In certain embodiments, the lipid portion of the lipid formulation comprises about, but is not necessarily limited to, 40 mol % to about 60 mol % of the ionizable cationic lipid, about 4 mol % to about 16 mol % DSPC, about 30 mol % to about 47 mol % cholesterol, and about 0.5 mol % to about 3 mol % PEG2000-DMG.

In certain embodiments, the lipid portion of the lipid formulation may comprise, but is not necessarily limited to, about 42 mol % to about 58 mol % of the ionizable cationic lipid, about 6 mol % to about 14 mol % DSPC, about 32 mol % to about 44 mol % cholesterol, and about 1 mol % to about 2 mol % PEG2000-DMG.

In certain embodiments, the lipid portion of the lipid formulation may comprise, but is not necessarily limited to, about 45 mol % to about 55 mol % of the ionizable cationic lipid, about 8 mol % to about 12 mol % DSPC, about 35 mol % to about 42 mol % cholesterol, and about 1.25 mol % to about 1.75 mol % PEG2000-DMG.

The percentage of helper lipid present in the lipid formulation is a target amount, and the actual amount of helper lipid present in the formulation may vary, for example, by ±5 mol %.

A lipid formulation that includes a cationic lipid compound or ionizable cationic lipid compound may be on a molar basis about 30-70% cationic lipid compound, about 25-40% cholesterol, about 2-15% helper lipid, and about 0.5-5% of a polyethylene glycol (PEG) lipid, wherein the percent is of the total lipid present in the formulation. In some aspects, the composition is about 40-65% cationic lipid compound, about 25-35% cholesterol, about 3-9% helper lipid, and about 0.5-3% of a PEG-lipid, wherein the percent is of the total lipid present in the formulation.

The formulation may be a lipid particle formulation, for example containing 8-30% nucleic acid compound, 5-30% helper lipid, and 0-20% cholesterol; 4-25% cationic lipid, 4-25% helper lipid, 2-25% cholesterol, 10-35% cholesterol-PEG, and 5% cholesterol-amine; or 2-30% cationic lipid, 2-30% helper lipid, 1-15% cholesterol, 2-35% cholesterol-PEG, and 1-20% cholesterol-amine; or up to 90% cationic lipid and 2-10% helper lipids, or even 100% cationic lipid.

### Lipid Conjugates

The lipid formulations described herein may further comprise a lipid conjugate. The conjugated lipid is useful in that it prevents the aggregation of particles. Suitable conjugated lipids include, but are not limited to, PEG-lipid conjugates, cationic-polymer-lipid conjugates, and mixtures thereof. Furthermore, lipid delivery vehicles can be used for specific targeting by attaching ligands (e.g., antibodies, peptides, and carbohydrates) to its surface or to the terminal end of the attached PEG chains (Front Pharmacol. 2015 Dec. 1; 6:286).

In some aspects, the lipid conjugate is a PEG-lipid. The inclusion of polyethylene glycol (PEG) in a lipid formulation as a coating or surface ligand, a technique referred to as PEGylation, helps to protect nanoparticles from the immune system and their escape from RES uptake (Nanomedicine (Lond). 2011 June; 6(4):715-28). PEGylation has been used to stabilize lipid formulations and their payloads through physical, chemical, and biological mechanisms. Detergent-like PEG lipids (e.g., PEG-DSPE) can enter the lipid formulation to form a hydrated layer and steric barrier on the surface. Based on the degree of PEGylation, the surface layer can be generally divided into two types, brush-like and mushroom-like layers. For PEG-DSPE-stabilized formulations, PEG will take on the mushroom conformation at a low degree of PEGylation (usually less than 5 mol %) and will shift to brush conformation as the content of PEG-DSPE is increased past a certain level (Journal of Nanomaterials. 2011; 2011:12). PEGylation leads to a significant increase in the circulation half-life of lipid formulations (Annu. Rev. Biomed. Eng. 2011 Aug. 15; 13( ):507-30; J. Control Release. 2010 Aug. 3; 145(3):178-81).

Examples of PEG-lipids include, but are not limited to, PEG coupled to dialkyloxypropyls (PEG-DAA), PEG coupled to diacylglycerol (PEG-DAG), methoxypolyethyleneglycol (PEG-DMG or PEG2000-DMG), PEG coupled to phospholipids such as phosphatidylethanolamine (PEG-PE), PEG conjugated to ceramides, PEG conjugated to cholesterol or a derivative thereof, and mixtures thereof.

PEG is a linear, water-soluble polymer of ethylene PEG repeating units with two terminal hydroxyl groups. PEGs are classified by their molecular weights and include the following: monomethoxypolyethylene glycol (MePEG-OH), monomethoxypolyethylene glycol-succinate (MePEG-S), monomethoxypolyethylene glycol-succinimidyl succinate (MePEG-S—NHS), monomethoxypolyethylene glycol-amine (MePEG-NH2), monomethoxypolyethylene glycol-tresylate (MePEG-TRES), monomethoxypolyethylene glycol-imidazolyl-carbonyl (MePEG-IM), as well as such compounds containing a terminal hydroxyl group instead of a terminal methoxy group (e.g., HO-PEG-S, HO-PEG-S—NHS, HO-PEG-NH2).

The PEG moiety of the PEG-lipid conjugates described herein may comprise an average molecular weight ranging from about 550 daltons to about 10,000 daltons. In certain aspects, the PEG moiety has an average molecular weight of from about 750 daltons to about 5,000 daltons (e.g., from about 1,000 daltons to about 5,000 daltons, from about 1,500 daltons to about 3,000 daltons, from about 750 daltons to about 3,000 daltons, from about 750 daltons to about 2,000 daltons). In some aspects, the PEG moiety has an average molecular weight of about 2,000 daltons or about 750 daltons. The average molecular weight may be any value or subvalue within the recited ranges, including endpoints.

In certain aspects, the PEG can be optionally substituted by an alkyl, alkoxy, acyl, or aryl group. The PEG can be conjugated directly to the lipid or may be linked to the lipid via a linker moiety. Any linker moiety suitable for coupling the PEG to a lipid can be used including, e.g., non-ester-containing linker moieties and ester-containing linker moieties. In one aspect, the linker moiety is a non-ester-containing linker moiety. Exemplary non-ester-containing linker moieties include, but are not limited to, amido (—C(O)NH—), amino (—NR—), carbonyl (—C(O)—), carbamate (—NHC(O)O—), urea (—NHC(O)NH—), disulfide (—S—S—), ether (—O—), succinyl (—(O)CCH2CH2C(O)—), succinamidyl (—NHC(O)CH2CH2C(O)NH—), ether, as well as combinations thereof (such as a linker containing both a carbamate linker moiety and an amido linker moiety). In one aspect, a carbamate linker is used to couple the PEG to the lipid.

In some aspects, an ester-containing linker moiety is used to couple the PEG to the lipid. Exemplary ester-containing linker moieties include, e.g., carbonate (—OC(O)O—), succinoyl, phosphate esters (—O—(O)POH—O—), sulfonate esters, and combinations thereof.

Phosphatidylethanolamines having a variety of acyl chain groups of varying chain lengths and degrees of saturation can be conjugated to PEG to form the lipid conjugate. Such phosphatidylethanolamines are commercially available or can be isolated or synthesized using conventional techniques known to those of skill in the art. Phosphatidylethanolamines containing saturated or unsaturated fatty acids with carbon chain lengths in the range of C₁₀ to C₂₀ are preferred. Phosphatidylethanolamines with mono- or di-unsaturated fatty acids and mixtures of saturated and unsaturated fatty acids can also be used. Suitable phosphatidylethanolamines include, but are not limited to, dimyristoyl-phosphatidylethanolamine (DMPE), dipalmitoyl-phosphatidylethanolamine (DPPE), dioleoyl-phosphatidylethanolamine (DOPE), and distearoyl-phosphatidylethanolamine (DSPE).

In some aspects, the PEG-DAA conjugate is a PEG-didecyloxypropyl (C10) conjugate, a PEG-dilauryloxypropyl (C12) conjugate, a PEG-dimyristyloxypropyl (C14) conjugate, a PEG-dipalmityloxypropyl (C16) conjugate, or a PEG-distearyloxypropyl (C18) conjugate. In some aspects, the PEG has an average molecular weight of about 750 or about 2,000 daltons. In some aspects, the terminal hydroxyl group of the PEG is substituted with a methyl group.

In addition to the foregoing, other hydrophilic polymers can be used in place of PEG. Examples of suitable polymers that can be used in place of PEG include, but are not limited to, polyvinylpyrrolidone, polymethyloxazoline, polyethyloxazoline, polyhydroxypropyl, methacrylamide, polymethacrylamide, and polydimethylacrylamide, polylactic acid, polyglycolic acid, and derivatized celluloses such as hydroxymethylcellulose or hydroxyethylcellulose.

In some aspects, the lipid conjugate (e.g., PEG-lipid) comprises from about 0.1 mol % to about 2 mol %, from about 0.5 mol % to about 2 mol %, from about 1 mol % to about 2 mol %, from about 0.6 mol % to about 1.9 mol %, from about 0.7 mol % to about 1.8 mol %, from about 0.8 mol % to about 1.7 mol %, from about 0.9 mol % to about 1.6 mol %, from about 0.9 mol % to about 1.8 mol %, from about 1 mol % to about 1.8 mol %, from about 1 mol % to about 1.7 mol %, from about 1.2 mol % to about 1.8 mol %, from about 1.2 mol % to about 1.7 mol %, from about 1.3 mol % to about 1.6 mol %, or from about 1.4 mol % to about 1.6 mol % (or any fraction thereof or range therein) of the total lipid present in the lipid formulation. In other embodiments, the lipid conjugate (e.g., PEG-lipid) comprises about 0.5%, 0.6%, 0.7%, 0.8%, 0.9%, 1.0%, 1.2%, 1.3%, 1.4%, 1.5%, 1.6%, 1.7%, 1.8%, 1.9%, 2.0%, 2.5%, 3.0%, 3.5%, 4.0%, 4.5%, or 5%, (or any fraction thereof or range therein) of the total lipid present in the lipid formulation. The amount may be any value or subvalue within the recited ranges, including endpoints.

The percentage of lipid conjugate (e.g., PEG-lipid) present in the lipid formulations of the disclosure is a target amount, and the actual amount of lipid conjugate present in the formulation may vary, for example, by ±0.5 mol %. One of ordinary skill in the art will appreciate that the concentration of the lipid conjugate can be varied depending on the lipid conjugate employed and the rate at which the lipid formulation is to become fusogenic.

In some embodiments, the lipid formulation for any of the compositions described herein comprises a lipoplex, a liposome, a lipid nanoparticle, a polymer-based particle, an exosome, a lamellar body, a micelle, or an emulsion.

### Mechanism of Action for Cellular Uptake of Lipid Formulations

In some aspects, lipid formulations for the intracellular delivery of nucleic acids, particularly liposomes, cationic liposomes, and lipid nanoparticles, are designed for cellular uptake by penetrating target cells through exploitation of the target cells' endocytic mechanisms where the contents of the lipid delivery vehicle are delivered to the cytosol of the target cell. (Nucleic Acid Therapeutics, 28(3):146-157, 2018). Prior to endocytosis, functionalized ligands such as PEG-lipid at the surface of the lipid delivery vehicle are shed from the surface, which triggers internalization into the target cell. During endocytosis, some part of the plasma membrane of the cell surrounds the vector and engulfs it into a vesicle that then pinches off from the cell membrane, enters the cytosol and ultimately enters and moves through the endolysosomal pathway. For ionizable cationic lipid-containing delivery vehicles, the increased acidity as the endosome ages results in a vehicle with a strong positive charge on the surface. Interactions between the delivery vehicle and the endosomal membrane then result in a membrane fusion event that leads to cytosolic delivery of the payload. For RNA payloads, the cell's own internal translation processes will then translate the RNA into the encoded protein. The encoded protein can further undergo postranslational processing, including transportation to a targeted organelle or location within the cell or excretion from the cell.

By controlling the composition and concentration of the lipid conjugate, one can control the rate at which the lipid conjugate exchanges out of the lipid formulation and, in turn, the rate at which the lipid formulation becomes fusogenic. In addition, other variables including, e.g., pH, temperature, or ionic strength, can be used to vary and/or control the rate at which the lipid formulation becomes fusogenic. Other methods which can be used to control the rate at which the lipid formulation becomes fusogenic will become apparent to those of skill in the art upon reading this disclosure. Also, by controlling the composition and concentration of the lipid conjugate, one can control the liposomal or lipid particle size.

### Lipid Formulation Manufacture

There are many different methods for the preparation of lipid formulations comprising a nucleic acid. (Curr. Drug Metabol. 2014, 15, 882-892; Chem. Phys. Lipids 2014, 177, 8-18; Int. J. Pharm. Stud. Res. 2012, 3, 14-20). The techniques of thin film hydration, double emulsion, reverse phase evaporation, microfluidic preparation, dual assymetric centrifugation, ethanol injection, detergent dialysis, spontaneous vesicle formation by ethanol dilution, and encapsulation in preformed liposomes are briefly described herein.

### Thin Film Hydration

In Thin Film Hydration (TFH) or the Bangham method, the lipids are dissolved in an organic solvent, then evaporated through the use of a rotary evaporator leading to a thin lipid layer formation. After the layer hydration by an aqueous buffer solution containing the compound to be loaded, Multilamellar Vesicles (MLVs) are formed, which can be reduced in size to produce Small or Large Unilamellar vesicles (LUV and SUV) by extrusion through membranes or by the sonication of the starting MLV.

### Double Emulsion

Lipid formulations can also be prepared through the Double Emulsion technique, which involves lipids dissolution in a water/organic solvent mixture. The organic solution, containing water droplets, is mixed with an excess of aqueous medium, leading to a water-in-oil-in-water (W/O/W) double emulsion formation. After mechanical vigorous shaking, part of the water droplets collapse, giving Large Unilamellar Vesicles (LUVs).

### Reverse Phase Evaporation

The Reverse Phase Evaporation (REV) method also allows one to achieve LUVs loaded with nucleic acid. In this technique a two-phase system is formed by phospholipids dissolution in organic solvents and aqueous buffer. The resulting suspension is then sonicated briefly until the mixture becomes a clear one-phase dispersion. The lipid formulation is achieved after the organic solvent evaporation under reduced pressure. This technique has been used to encapsulate different large and small hydrophilic molecules including nucleic acids.

### Microfluidic Preparation

The Microfluidic method, unlike other bulk techniques, gives the possibility of controlling the lipid hydration process. The method can be classified in continuous-flow microfluidic and droplet-based microfluidic, according to the way in which the flow is manipulated. In the microfluidic hydrodynamic focusing (MHF) method, which operates in a continuous flow mode, lipids are dissolved in isopropyl alcohol which is hydrodynamically focused in a microchannel cross junction between two aqueous buffer streams. Vesicles size can be controlled by modulating the flow rates, thus controlling the lipids solution/buffer dilution process. The method can be used for producing oligonucleotide (ON) lipid formulations by using a microfluidic device consisting of three-inlet and one-outlet ports.

### Dual Asymmetric Centrifugation

Dual Asymmetric Centrifugation (DAC) differs from more common centrifugation as it uses an additional rotation around its own vertical axis. An efficient homogenization is achieved due to the two overlaying movements generated: the sample is pushed outwards, as in a normal centrifuge, and then it is pushed towards the center of the vial due to the additional rotation. By mixing lipids and an NaCl-solution a viscous vesicular phospholipid gel (VPC) is achieved, which is then diluted to obtain a lipid formulation dispersion. The lipid formulation size can be regulated by optimizing DAC speed, lipid concentration and homogenization time.

### Ethanol Injection

The Ethanol Injection (EI) method can be used for nucleic acid encapsulation. This method provides the rapid injection of an ethanolic solution, in which lipids are dissolved, into an aqueous medium containing nucleic acids to be encapsulated, through the use of a needle. Vesicles are spontaneously formed when the phospholipids are dispersed throughout the medium.

### Detergent Dialysis

The Detergent dialysis method can be used to encapsulate nucleic acids. Briefly lipid and plasmid are solubilized in a detergent solution of appropriate ionic strength, after removing the detergent by dialysis, a stabilized lipid formulation is formed. Unencapsulated nucleic acid is then removed by ion-exchange chromatography and empty vesicles by sucrose density gradient centrifugation. The technique is highly sensitive to the cationic lipid content and to the salt concentration of the dialysis buffer, and the method is also difficult to scale.

### Spontaneous Vesicle Formation by Ethanol Dilution

Stable lipid formulations can also be produced through the Spontaneous Vesicle Formation by Ethanol Dilution method in which a stepwise or dropwise ethanol dilution provides the instantaneous formation of vesicles loaded with nucleic acid by the controlled addition of lipid dissolved in ethanol to a rapidly mixing aqueous buffer containing the nucleic acid.

### Encapsulation in Preformed Liposomes

The entrapment of nucleic acids can also be obtained starting with preformed liposomes through two different methods: (1) A simple mixing of cationic liposomes with nucleic acids which gives electrostatic complexes called “lipoplexes”, where they can be successfully used to transfect cell cultures, but are characterized by their low encapsulation efficiency and poor performance in vivo; and (2) a liposomal destabilization, slowly adding absolute ethanol to a suspension of cationic vesicles up to a concentration of 40% v/v followed by the dropwise addition of nucleic acids achieving loaded vesicles; however, the two main steps characterizing the encapsulation process are too sensitive, and the particles have to be downsized.

### Excipients

The pharmaceutical compositions disclosed herein can be formulated using one or more excipients to: (1) increase stability; (2) increase cell transfection; (3) permit a sustained or delayed release (e.g., from a depot formulation of the polynucleotide, primary construct, or RNA); (4) alter the biodistribution (e.g., target the polynucleotide, primary construct, or RNA to specific tissues or cell types); (5) increase the translation of encoded protein in vivo; and/or (6) alter the release profile of encoded protein in vivo.

The pharmaceutical compositions described herein may be prepared by any method known or hereafter developed in the art of pharmacology. In general, such preparatory methods include the step of associating the active ingredient (i.e., nucleic acid) with an excipient and/or one or more other accessory ingredients. A pharmaceutical composition in accordance with the present disclosure may be prepared, packaged, and/or sold in bulk, as a single unit dose, and/or as a plurality of single unit doses.

Pharmaceutical compositions may additionally comprise a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired.

In addition to traditional excipients such as any and all solvents, dispersion media, diluents, or other liquid vehicles, dispersion or suspension aids, surface active agents, isotonic agents, thickening or emulsifying agents, preservatives, excipients of the present disclosure can include, without limitation, liposomes, lipid nanoparticles, polymers, lipoplexes, core-shell nanoparticles, peptides, proteins, cells transfected with primary DNA construct, or RNA (e.g., for transplantation into a subject), hyaluronidase, nanoparticle mimics and combinations thereof.

Accordingly, the pharmaceutical compositions described herein can include one or more excipients, each in an amount that together increases the stability of the nucleic acid in the lipid formulation, increases cell transfection by the nucleic acid, increases the expression of the encoded protein, and/or alters the release profile of encoded proteins. Further, the RNA of the present disclosure may be formulated using self-assembled nucleic acid nanoparticles.

Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; incorporated herein by reference in its entirety). The use of a conventional excipient medium may be contemplated within the scope of the embodiments of the present disclosure, except insofar as any conventional excipient medium may be incompatible with a substance or its derivatives, such as by producing any undesirable biological effect or otherwise interacting in a deleterious manner with any other component(s) of the pharmaceutical composition.

The pharmaceutical compositions of this disclosure may further contain as pharmaceutically acceptable carriers substances as required to approximate physiological conditions, such as pH adjusting and buffering agents, tonicity adjusting agents, and wetting agents, for example, sodium acetate, sodium lactate, sodium chloride, potassium chloride, calcium chloride, sorbitan monolaurate, triethanolamine oleate, and mixtures thereof. For solid compositions, conventional nontoxic pharmaceutically acceptable carriers can be used which include, for example, pharmaceutical grades of mannitol, lactose, starch, magnesium stearate, sodium saccharin, talcum, cellulose, glucose, sucrose, magnesium carbonate, and the like.

In certain embodiments of the disclosure, the RNA-lipid formulation may be administered in a time release formulation, for example in a composition which includes a slow release polymer. The active agent can be prepared with carriers that will protect against rapid release, for example a controlled release vehicle such as a polymer, microencapsulated delivery system, or a bioadhesive gel. Prolonged delivery of the RNA, in various compositions of the disclosure can be brought about by including in the composition agents that delay absorption, for example, aluminum monostearate hydrogels and gelatin.

### Methods of Inducing Immune Responses

Provided herein, in some embodiments, are methods of inducing an immune response in a subject. Any type of immune response can be induced using the methods provided herein, including adaptive and innate immune responses. In one aspect, immune responses induced using the methods provided herein include an antibody response, a cellular immune response, or both an antibody response and a cellular immune response.

Methods of inducing an immune response provided herein include administering to a subject an effective amount of any nucleic acid molecule provided herein. In one aspect, methods of inducing an immune response include administering to a subject an effective amount of any composition comprising a nucleic acid molecule and a lipid provided herein. In another aspect, methods of inducing an immune response include administering to a subject an effective amount of any pharmaceutical composition comprising a nucleic acid molecule and a lipid formulation provided herein. In some aspects, nucleic acid molecules, compositions, and pharmaceutical composition provided here are vaccines that can elicit a protective or a therapeutic immune response, for example.

As used herein, the term “subject” refers to any individual or patient on which the methods disclosed herein are performed. The term “subject” can be used interchangeably with the term “individual” or “patient.” The subject can be a human, although the subject may be an animal, as will be appreciated by those in the art. Thus, other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject. As used herein, the term “effective amount” or “therapeutically effective amount” refers to that amount of a nucleic acid molecule, composition, or pharmaceutical composition described herein that is sufficient to effect the intended application, including but not limited to inducing an immune response and/or disease treatment, as defined herein. The therapeutically effective amount may vary depending upon the intended application (e.g., inducing an immune response, treatment, application in vivo), or the subject or patient and disease condition being treated, e.g., the weight and age of the subject, the species, the severity of the disease condition, the manner of administration and the like, which can readily be determined by one of ordinary skill in the art. The term also applies to a dose that will induce a particular response in a target cell. The specific dose will vary depending on the particular nucleic acid molecule, composition, or pharmaceutical composition chosen, the dosing regimen to be followed, whether it is administered in combination with other compounds, timing of administration, the tissue to which it is administered, and the physical delivery system in which it is carried.

Exemplary doses of nucleic acid molecules that can be administered include about 0.01 μg, about 0.02 μg, about 0.03 μg, about 0.04 μg, about 0.05 μg, about 0.06 μg, about 0.07 μg, about 0.08 μg, about 0.09 μg, about 0.1 μg, about 0.2 μg, about 0.3 μg, about 0.4 μg, about 0.5 μg, about 0.6 μg, about 0.7 μg, about 0.8 μg, about 0.9 μg, about 1.0 μg, about 1.5 μg, about 2.0 μg, about 2.5 μg, about 3.0 μg, about 3.5 μg, about 4.0 μg, about 4.5 μg, about 5.0 μg, about 5.5 μg, about 6.0 μg, about 6.5 μg, about 7.0 μg, about 7.5 μg, about 8.0 μg, about 8.5 μg, about 9.0 μg, about 9.5 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, about 100 μg, about 125 μg, about 150 μg, about 175 μg, about 200 μg, about 250 μg, about 300 μg, about 350 μg, about 400 μg, about 450 μg, about 500 μg, about 600 μg, about 700 μg, about 800 μg, about 900 μg, about 1,000 μg, or more, and any number or range in between. In one aspect, the nucleic acid molecules are RNA molecules. In another aspect, the nucleic acid molecules are DNA molecules. Nucleic acid molecules can have a unit dosage comprising about 0.01 μg to about 1,000 μg or more nucleic acid in a single dose.

In some aspects, compositions provided herein that can be administered include about 0.01 μg, about 0.02 μg, about 0.03 μg, about 0.04 μg, about 0.05 μg, about 0.06 μg, about 0.07 μg, about 0.08 μg, about 0.09 μg, about 0.1 μg, about 0.2 μg, about 0.3 μg, about 0.4 μg, about 0.5 μg, about 0.6 μg, about 0.7 μg, about 0.8 μg, about 0.9 μg, about 1.0 μg, about 1.5 μg, about 2.0 μg, about 2.5 μg, about 3.0 μg, about 3.5 μg, about 4.0 μg, about 4.5 μg, about 5.0 μg, about 5.5 μg, about 6.0 μg, about 6.5 μg, about 7.0 μg, about 7.5 μg, about 8.0 μg, about 8.5 μg, about 9.0 μg, about 9.5 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, about 100 μg, about 125 μg, about 150 μg, about 175 μg, about 200 μg, about 250 μg, about 300 μg, about 350 μg, about 400 μg, about 450 μg, about 500 μg, about 600 μg, about 700 μg, about 800 μg, about 900 μg, about 1,000 μg, or more, and any number or range in between, nucleic acid and lipid. In other aspects, pharmaceutical compositions provided herein that can be administered include about 0.01 μg, about 0.02 μg, about 0.03 μg, about 0.04 μg, about 0.05 μg, about 0.06 μg, about 0.07 μg, about 0.08 μg, about 0.09 μg, about 0.1 μg, about 0.2 μg, about 0.3 μg, about 0.4 μg, about 0.5 μg, about 0.6 μg, about 0.7 μg, about 0.8 μg, about 0.9 μg, about 1.0 μg, about 1.5 μg, about 2.0 μg, about 2.5 μg, about 3.0 μg, about 3.5 μg, about 4.0 μg, about 4.5 μg, about 5.0 μg, about 5.5 μg, about 6.0 μg, about 6.5 μg, about 7.0 μg, about 7.5 μg, about 8.0 μg, about 8.5 μg, about 9.0 μg, about 9.5 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, about 100 μg, about 125 μg, about 150 μg, about 175 μg, about 200 μg, about 250 μg, about 300 μg, about 350 μg, about 400 μg, about 450 μg, about 500 μg, about 600 μg, about 700 μg, about 800 μg, about 900 μg, about 1,000 μg, or more, and any number or range in between, nucleic acid and lipid formulation.

In one aspect, compositions provided herein can have a unit dosage comprising about 0.01 μg to about 1,000 μg or more nucleic acid and lipid in a single dose. In another aspect, pharmaceutical compositions provided herein can have a unit dosage comprising about 0.01 μg to about 1,000 μg or more nucleic acid and lipid formulation in a single dose. A vaccine unit dosage can correspond to the unit dosage of nucleic acid molecules, compositions, or pharmaceutical compositions provided herein and that can be administered to a subject. In one aspect, vaccine compositions of the instant disclosure have a unit dosage comprising about 0.01 μg to about 1,000 μg or more nucleic acid and lipid formulation in a single dose. In another aspect, vaccine compositions of the instant disclosure have a unit dosage comprising about 0.01 μg to about 50 μg nucleic acid and lipid formulation in a single dose. In yet another aspect, vaccine compositions of the instant disclosure have a unit dosage comprising about 0.2 μg to about 20 μg nucleic acid and lipid formulation in a single dose.

A dosage form of the composition of this disclosure can be solid, which can be reconstituted in a liquid prior to administration. The solid can be administered as a powder. The solid can be in the form of a capsule, tablet, or gel. In some embodiments, the pharmaceutical composition comprises a nucleic acid lipid formulation that has been lyophilized. In some embodiments, the lyophilized composition may comprise one or more lyoprotectants, such as, including but not necessarily limited to, glucose, trehalose, sucrose, maltose, lactose, mannitol, inositol, hydroxypropyl-β-cyclodextrin, and/or polyethylene glycol. In some embodiments, the lyophilized composition comprises a poloxamer, potassium sorbate, sucrose, or any combination thereof. In specific embodiments, the poloxamer is poloxamer 188. In some embodiments, the lyophilized compositions described herein may comprise about 0.01 to about 1.0% w/w of a poloxamer. In some embodiments, the lyophilized compositions described herein may comprise about 1.0 to about 5.0% w/w of potassium sorbate. The percentages may be any value or subvalue within the recited ranges, including endpoints.

In some embodiments, the lyophilized composition may comprise about 0.01 to about 1.0% w/w of the nucleic acid molecule. In some embodiments, the composition may comprise about 1.0 to about 5.0% w/w lipids. In some embodiments, the composition may comprise about 0.5 to about 2.5% w/w of TRIS buffer. In some embodiments, the composition may comprise about 0.75 to about 2.75% w/w of NaCl. In some embodiments, the composition may comprise about 85 to about 95% w/w of a sugar. The percentages may be any value or subvalue within the recited ranges, including endpoints.

In a preferred embodiment, the dosage form of the pharmaceutical compositions described herein can be a liquid suspension of self-replicating RNA lipid nanoparticles described herein. In some embodiments, the liquid suspension is in a buffered solution. In some embodiments, the buffered solution comprises a buffer selected from the group consisting of HEPES, MOPS, TES, and TRIS. In some embodiments, the buffer has a pH of about 7.4. In some preferred embodiments, the buffer is HEPES. In some further embodiments, the buffered solution further comprises a cryoprotectant. In some embodiments, the cryoprotectant is selected from a sugar and glycerol or a combination of a sugar and glycerol. In some embodiments, the sugar is a dimeric sugar. In some embodiments, the sugar is sucrose. In some preferred embodiments, the buffer comprises HEPES, sucrose, and glycerol at a pH of 7.4. In certain embodiments, the composition comprises a HEPES, MOPS, TES, or TRIS buffer at a pH of about 7.0 to about 8.5. In some embodiments, the HEPES, MOPS, TES, or TRIS buffer may at a concentration ranging from 7 mg/ml to about 15 mg/ml. The pH or concentration may be any value or subvalue within the recited ranges, including endpoints.

In some embodiments, the suspension is frozen during storage and thawed prior to administration. In some embodiments, the suspension is frozen at a temperature below about 70° C. In some embodiments, the suspension is diluted with sterile water during intravenous administration. In some embodiments, intravenous administration comprises diluting the suspension with about 2 volumes to about 6 volumes of sterile water. In some embodiments, the suspension comprises about 0.1 mg to about 3.0 mg self-replicating RNA/mL, about 15 mg/mL to about 25 mg/mL of an ionizable cationic lipid, about 0.5 mg/mL to about 2.5 mg/mL of a PEG-lipid, about 1.8 mg/mL to about 3.5 mg/mL of a helper lipid, about 4.5 mg/mL to about 7.5 mg/mL of a cholesterol, about 7 mg/mL to about 15 mg/mL of a buffer, about 2.0 mg/mL to about 4.0 mg/mL of NaCl, about 70 mg/mL to about 110 mg/mL of sucrose, and about 50 mg/mL to about 70 mg/mL of glycerol. In some embodiments, a lyophilized self-replicating RNA-lipid nanoparticle formulation can be resuspended in a buffer as described herein.

In some embodiments, the compositions of the disclosure are administered to a subject such that a self-replicating RNA concentration of at least about 0.05 mg/kg, at least about 0.1 mg/kg, at least about 0.5 mg/kg, at least about 1.0 mg/kg, at least about 2.0 mg/kg, at least about 3.0 mg/kg, at least about 4.0 mg/kg, at least about 5.0 mg/kg of body weight is administered in a single dose or as part of single treatment cycle. In some embodiments, the compositions of the disclosure are administered to a subject such that a total amount of at least about 0.1 mg, at least about 0.5 mg, at least about 1.0 mg, at least about 2.0 mg, at least about 3.0 mg, at least about 4.0 mg, at least about 5.0 mg, at least about 6.0 mg, at least about 7.0 mg, at least about 8.0 mg, at least about 9.0 mg, at least about 10 mg, at least about 15 mg, at least about 20 mg, at least about 25 mg, at least about 30 mg, at least about 35 mg, at least about 40 mg, at least about 45 mg, at least about 50 mg, at least about 55 mg, at least about 60 mg, at least about 65 mg, at least about 70 mg, at least about 75 mg, at least about 80 mg, at least about 85 mg, at least about 90 mg, at least about 95 mg, at least about 100 mg, at least about 105 mg, at least about 110 mg, at least about 115 mg, at least about 120 mg, or at least about 125 mg self-replicating RNA is administered in one or more doses up to a maximum dose of about 300 mg, about 350 mg, about 400 mg, about 450 mg, or about 500 mg self-replicating RNA.

Any route of administration can be included in methods provided herein. In some aspects, nucleic acid molecules, compositions, and pharmaceutical compositions provided herein are administered intramuscularly, subcutaneously, intradermally, transdermally, intranasally, orally, sublingually, intravenously, intraperitoneally, topically, by aerosol, or by a pulmonary route, such as by inhalation or by nebulization, for example. In some embodiments, the pharmaceutical compositions described are administered systemically. Suitable routes of administration include, for example, oral, rectal, vaginal, transmucosal, pulmonary including intratracheal or inhaled, or intestinal administration; parenteral delivery, including intradermal, transdermal (topical), intramuscular, subcutaneous, intramedullary injections, as well as intrathecal, direct intraventricular, intravenous, intraperitoneal, or intranasal. In particular embodiments, the intramuscular administration is to a muscle selected from the group consisting of skeletal muscle, smooth muscle and cardiac muscle. In some embodiments, the pharmaceutical composition is administered intravenously.

Pharmaceutical compositions may be administered to any desired tissue. In some embodiments, the self-replicating RNA delivered is expressed in a tissue different from the tissue in which the lipid formulation or pharmaceutical composition was administered. In preferred embodiments, self-replicating RNA is delivered and expressed in the liver.

In other aspects, nucleic acid molecules, compositions, and pharmaceutical compositions provided herein are administered intramuscularly.

In some aspects, the subject in which an immune response is induced is a healthy subject. As used herein, the term “healthy subject” refers to a subject not having a condition or disease, including an infectious disease or cancer, for example, or not having a condition or disease against which an immune response is induced. Accordingly, in some aspects, a nucleic acid molecule, composition, or pharmaceutical composition provided herein is administered prophylactically to prevent an infectious disease or cancer, for example. In other aspects, the subject in which an immune response is induced has cancer. The subject may suffer from any cancer or have any tumor, including solid and liquid tumors. In one aspect, the cancer is kidney cancer, renal cancer, urinary bladder cancer, prostate cancer, uterine cancer, breast cancer, cervical cancer, ovarian cancer, lung cancer, liver cancer, stomach cancer, colon cancer, rectal cancer, oral cavity cancer, pharynx cancer, pancreatic cancer, thyroid cancer, melanoma, skin cancer, head and neck cancer, brain cancer, hematopoietic cancer, leukemia, lymphoma, bone cancer, or sarcoma. Accordingly, a nucleic acid molecule, composition, or pharmaceutical composition provided herein can be administered therapeutically, i.e., to treat a condition or disease, such as cancer, after the onset of the condition or disease.

As used herein, the terms “treat,” “treatment,” “therapy,” “therapeutic,” and the like refer to obtaining a desired pharmacologic and/or physiologic effect, including, but not limited to, alleviating, delaying or slowing the progression, reducing the effects or symptoms, preventing onset, inhibiting, ameliorating the onset of a diseases or disorder, obtaining a beneficial or desired result with respect to a disease, disorder, or medical condition, such as a therapeutic benefit and/or a prophylactic benefit. “Treatment,” as used herein, includes any treatment of a disease in a mammal, particularly in a human, and includes: (a) preventing the disease from occurring in a subject, including a subject which is predisposed to the disease or at risk of acquiring the disease but has not yet been diagnosed as having it; (b) inhibiting the disease, i.e., arresting its development; and (c) relieving the disease, i.e., causing regression of the disease. A therapeutic benefit includes eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. In some aspects, for prophylactic benefit, treatment or compositions for treatment, including pharmaceutical compositions, are administered to a subject at risk of developing a particular disease, or to a subject reporting one or more of the physiological symptoms of a disease, even though a diagnosis of this disease may not have been made. The methods of the present disclosure may be used with any mammal or other animal. In some aspects, treatment results in a decrease or cessation of symptoms. A prophylactic effect includes delaying or eliminating the appearance of a disease or condition, delaying or eliminating the onset of symptoms of a disease or condition, slowing, halting, or reversing the progression of a disease or condition, or any combination thereof.

Nucleic acid molecules, compositions, and pharmaceutical compositions provided herein can be administered once or multiple times. Accordingly, nucleic acid molecules, compositions, and pharmaceutical compositions provided herein can be administered one, two, three, four, five, six, seven, eight, nine, ten, or more times. Timing between two or more administrations can be one week, two weeks, three weeks, four weeks, five weeks, six weeks, seven weeks, eight weeks, nine weeks, weeks, ten weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or more weeks, and any number or range in between. In some aspects, timing between two or more administrations is one month, two months, three months, four months, five months, six months, seven months, eight months, nine months, ten months, 11 months, 12 months, 13 months, 14 months, 15 months, 16 months, 17 months, 18 months, 19 months, 20 months, 21 months, 22 months, 23 months, 24 months, or more months, and any number or range in between. In other aspects, timing between two or more administrations can be one year, two years, three years, four years, five years, six years, seven years, eight years, nine years, ten years, or more years, and any number or range in between, Timing between the first and any subsequent administration can be the same or different. In one aspect, nucleic acid molecules, compositions, or pharmaceutical compositions provided herein are administered once.

More than one nucleic acid molecule, composition, or pharmaceutical composition can be administered in the methods provided herein. In one aspect, two or more nucleic acid molecules, compositions, or pharmaceutical compositions provided herein are administered simultaneously. In another aspect, two or more nucleic acid molecules, compositions, or pharmaceutical compositions provided herein are administered sequentially. Simultaneous and sequential administrations can include any number and any combination of nucleic acid molecules, compositions, or pharmaceutical compositions provided herein. Multiple nucleic acid molecules, compositions, or pharmaceutical compositions that are administered together or sequentially can include transgenes encoding different antigenic proteins or fragments thereof. In this manner, immune responses against different antigenic targets can be induced. Two, three, four, five, six, seven, eight, nine, ten, or more nucleic acid molecules, compositions, or pharmaceutical compositions including transgenes encoding different antigenic proteins or fragments thereof can be administered simultaneously or sequentially. Any combination of nucleic acid molecules, compositions, and pharmaceutical compositions including any combination of transgenes can be administered simultaneously or sequentially. In some aspects, administration is simultaneous. In other aspects, administration is sequential. Timing between two or more administrations can be one week, two weeks, three weeks, four weeks, five weeks, six weeks, seven weeks, eight weeks, nine weeks, weeks, ten weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or more weeks, and any number or range in between. In some aspects, timing between two or more administrations is one month, two months, three months, four months, five months, six months, seven months, eight months, nine months, ten months, 11 months, 12 months, 13 months, 14 months, 15 months, months, 16 months, 17 months, 18 months, 19 months, 20 months, 21 months, 22 months, 23 months, 24 months, or more months, and any number or range in between. In other aspects, timing between two or more administrations can be one year, two years, three years, four years, five years, six years, seven years, eight years, nine years, ten years, or more years, and any number or range in between, Timing between the first and any subsequent administration can be the same or different. Nucleic acid molecules, compositions, and pharmaceutical compositions provided herein can be administered with any other vaccine or treatment.

Following administration of the composition to the subject, the protein product encoded by the self-replicating RNA of the disclosure (e.g., an antigen) is detectable in the target tissues for at least about one to seven days or longer. The amount of protein product necessary to achieve a therapeutic effect will vary depending on antibody titer necessary to generate an immunity to COVID-19 in the patient. For example, the protein product may be detectable in the target tissues at a concentration (e.g., a therapeutic concentration) of at least about 0.025-1.5 g/ml (e.g., at least about 0.050 g/ml, at least about 0.075 g/ml, at least about 0.1 g/ml, at least about 0.2 g/ml, at least about 0.3 g/ml, at least about 0.4 g/ml, at least about 0.5 g/ml, at least about 0.6 g/ml, at least about 0.7 g/ml, at least about 0.8 g/ml, at least about 0.9 g/ml, at least about 1.0 g/ml, at least about 1.1 g/ml, at least about 1.2 g/ml, at least about 1.3 g/ml, at least about 1.4 g/ml, or at least about 1.5 g/ml), for at least about 1,2, 3,4, 5, 6, 7, 8, 9, 10, 11, 12, 13, 14, 15, 16, 17, 18, 19, 20, 21, 22, 23, 24, 25, 26, 27, 28, 29, 30, 35, 40, 45 days or longer following administration of the composition to the subject.

In some embodiments, the composition described herein may be administered one time. In some embodiments, the composition described herein may be administered two times.

In some embodiments, the composition may be administered in the form of a booster dose, to a subject who was previously vaccinated against coronavirus.

In some embodiments, a pharmaceutical composition of the present disclosure is administered to a subject once per month. In some embodiments, a pharmaceutical composition of the present disclosure is administered to a subject twice per month. In some embodiments, a pharmaceutical composition of the present disclosure is administered to a subject three times per month. In some embodiments, a pharmaceutical composition of the present disclosure is administered to a subject four times per month.

Alternatively, the compositions of the present disclosure may be administered in a local rather than systemic manner, for example, via injection of the pharmaceutical composition directly into a targeted tissue, preferably in a depot or sustained release formulation. Local delivery can be affected in various ways, depending on the tissue to be targeted. For example, aerosols containing compositions of the present disclosure can be inhaled (for nasal, tracheal, or bronchial delivery); compositions of the present disclosure can be injected into the site of injury, disease manifestation, or pain, for example; compositions can be provided in lozenges for oral, tracheal, or esophageal application; can be supplied in liquid, tablet or capsule form for administration to the stomach or intestines, can be supplied in suppository form for rectal or vaginal application; or can even be delivered to the eye by use of creams, drops, or even injection. Formulations containing compositions of the present disclosure complexed with therapeutic molecules or ligands can even be surgically administered, for example in association with a polymer or other structure or substance that can allow the compositions to diffuse from the site of implantation to surrounding cells. Alternatively, they can be applied surgically without the use of polymers or supports.

### Combinations

The self-replicating RNA, formulations thereof, or encoded proteins described herein may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent. Preferably, the methods of treatment of the present disclosure encompass the delivery of pharmaceutical, prophylactic, diagnostic, or imaging compositions in combination with agents that may improve their bioavailability, reduce and/or modify their metabolism, inhibit their excretion, and/or modify their distribution within the body. As a non-limiting example, a self-replicating RNA of the disclosure may be used in combination with a pharmaceutical agent for immunizing or vaccinating a subject. In general, it is expected that agents utilized in combination with the presently disclosed self-replicating RNA and formulations thereof be utilized at levels that do not exceed the levels at which they are utilized individually. In some embodiments, the levels utilized in combination will be lower than those utilized individually. In one embodiment, the combinations, each or together may be administered according to the split dosing regimens as are known in the art.

### Ranges

Throughout this disclosure, various aspects can be presented in range format. It should be understood that any description in range format is merely for convenience and brevity and not meant to be limiting. Accordingly, the description of a range should be considered to have specifically disclosed all possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example 1, 2, 2.1, 2.2, 2.5, 3, 4, 4.75, 4.8, 4.85, 4.95, 5, 5.5, 5.75, 5.9, 5.00, and 6. This applies to a range of any breadth.

### Example 1

This example describes a comparison of design and expression of mRNA and self-replicating RNA (STARR™) platforms.

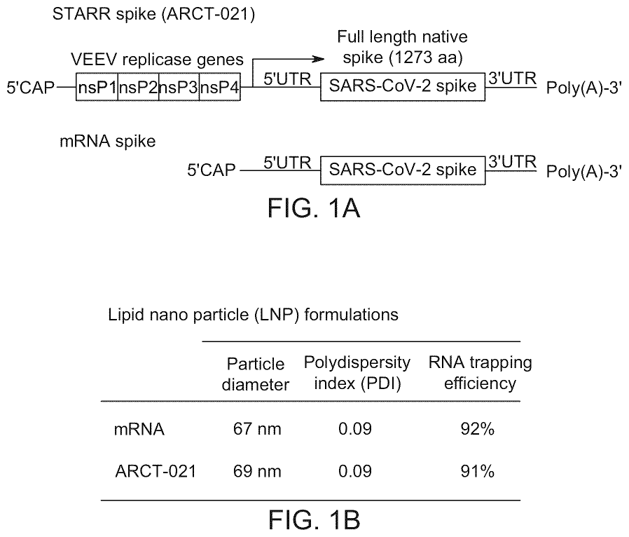

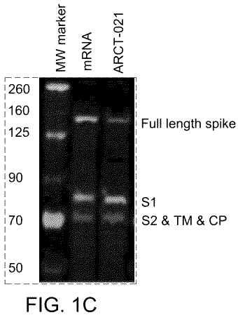

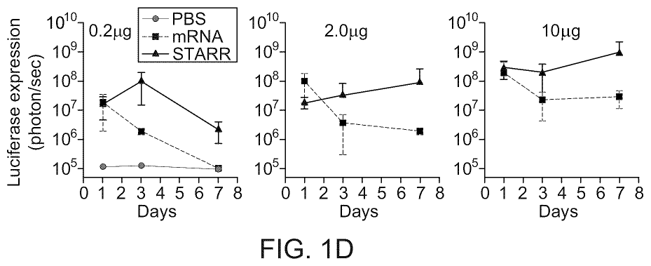

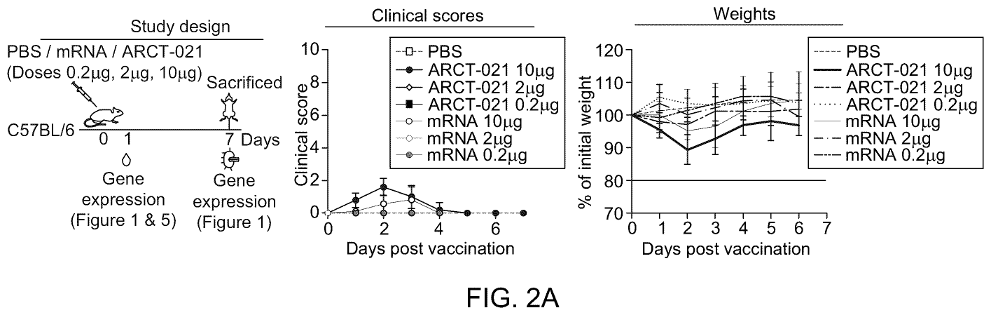

Both mRNA and STARR™ vaccine constructs were designed to encode the full-length SARS-CoV-2 S protein (1273 aa), with the STARR™ self-replicating RNA additionally encoding for the Venezuelan equine encephalitis virus (VEEV) replicase genes (FIG. 1A; STARR™ vaccine construct corresponding to an RNA having a sequence of SEQ ID NO:125, with U in place of T, referred to herein as “STARR™ SARS-CoV-2 RNA”; mRNA corresponding to a sequence of SEQ ID NO:126, with U in place of T and including a tobacco etch virus (TEV) 5′ UTR, a Xenopus beta-globin (Xbg) 3′ UTR, and a codon-optimized open reading frame encoding the SARS-CoV-2 glycoprotein). The characteristics of these different constructs was studied first. Constructs were encapsulated in the same LNP composition. Briefly, RNA constructs were encapsulated into lipid nanoparticles (LNPs) that included four lipid excipients (an ionizable cationic lipid, 1,2-distearoyl-sn-glycero-3-phosphocholine (DSPC), cholesterol, and PEG2000-DMG) dispersed in HEPES buffer (pH 8.0) containing sodium chloride and the cryoprotectants sucrose and glycerol. The ionizable cationic lipid had the following structure:

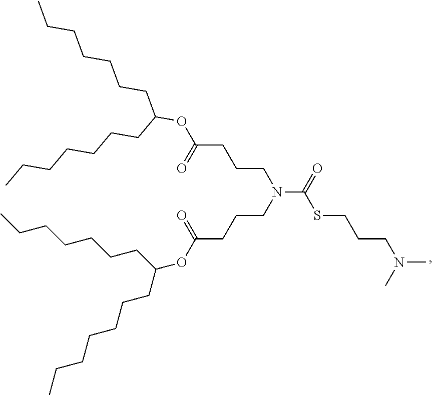 [SMILES: CCCCCCCC(CCCCCCC)OC(=O)CCCN(CCCC(=O)OC(CCCCCCC)CCCCCCC)C(=O)SCCCN(C)C]

Despite differences in RNA lengths of mRNA and the STARR™ SARS-CoV-2 RNA construct, the LNP diameter, polydispersity index and RNA trapping efficiency were similar (FIG. 1B). In vitro expression of the mRNA vaccine and the STARR™ SARS-CoV-2 RNA construct were confirmed in cell lysate 24 hours post-transfection through positive western blot detection of the S protein (FIG. 1C). It was also observed that both mRNA and STARR™ vaccines expressed a mixture of full-length S protein and cleaved S protein, i.e., S was cleaved into S1 and S2 transmembrane and cytoplasmic membrane domains (FIG. 1C). In vivo protein expression of the two RNA platforms in BALB/c mice was compared by using mRNA and STARR™ constructs that expressed a luciferase reporter (FIG. 1D). Animals injected with the mRNA vaccine construct showed high in vivo luciferase expression at day 1, although the expression levels declined over time. In contrast, luciferase expression in STARR™ injected mice showed sustained or even increased signals, apart from those given the 0.2 μg dose, until day 7 post-inoculation (FIG. 1D).

These data show that dose-for-dose, antigen expression was more prolonged with the STARR™ compared to the mRNA vaccine.

### Example 2

This example describes immune gene expression following the STARR™ construct and mRNA vaccination.

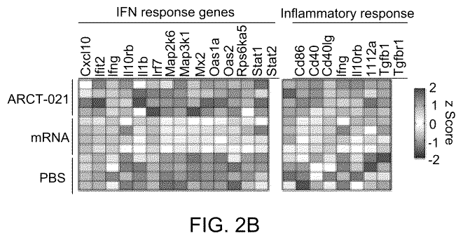

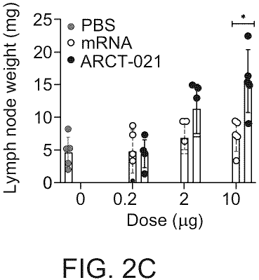

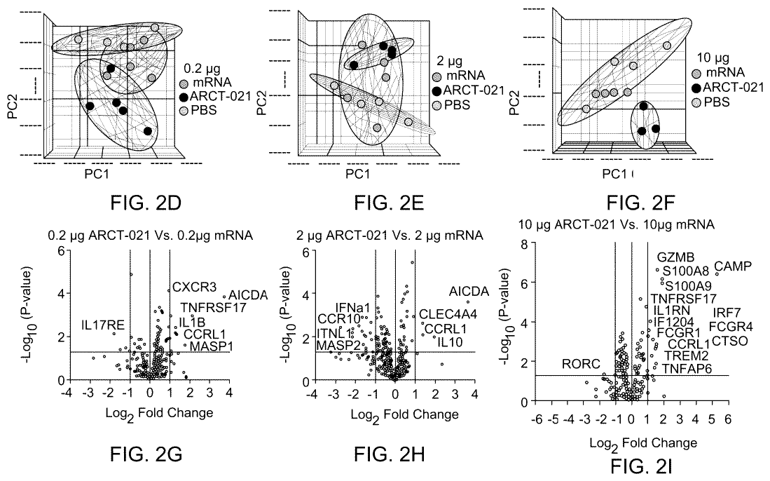

C57BL/6J mice were inoculated with STARR™ SARS-CoV-2 RNA (encoding the SARS-CoV-2 glycoprotein as described above (Example 1)) and mRNA vaccines at 0.2 μg, 2 μg and 10 μg doses or PBS control. No significant mean loss in animal weight occurred over the first 4 days, except for those that received 10 μg of STARR™ SARS-CoV-2 RNA (FIG. 2A). However, apart from weight loss, there were few other clinical signs as indicated by the minimal differences in clinical scores. Both weight and clinical scores improved after day 3 post vaccination.

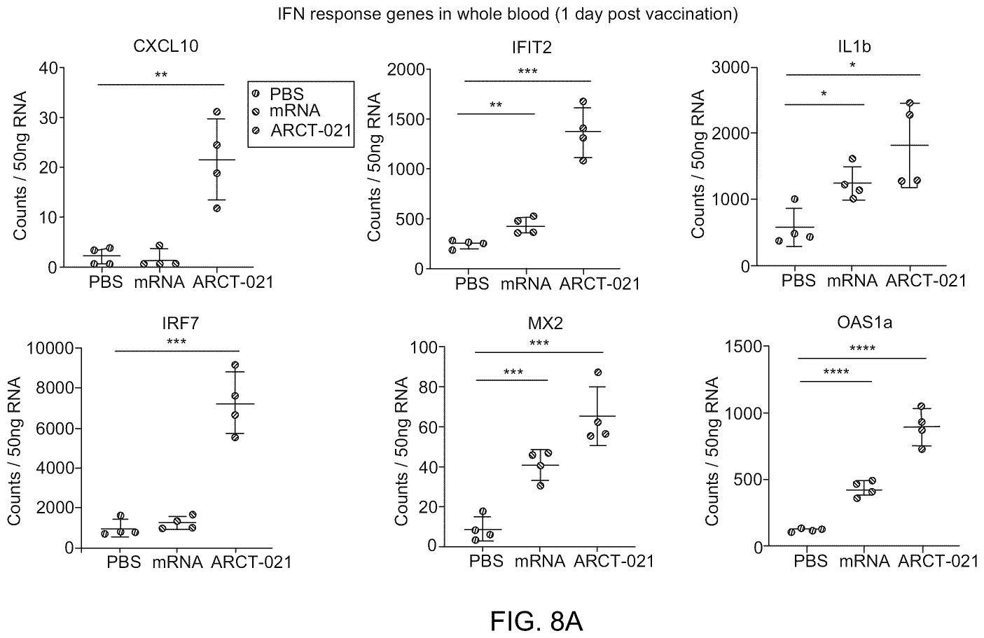

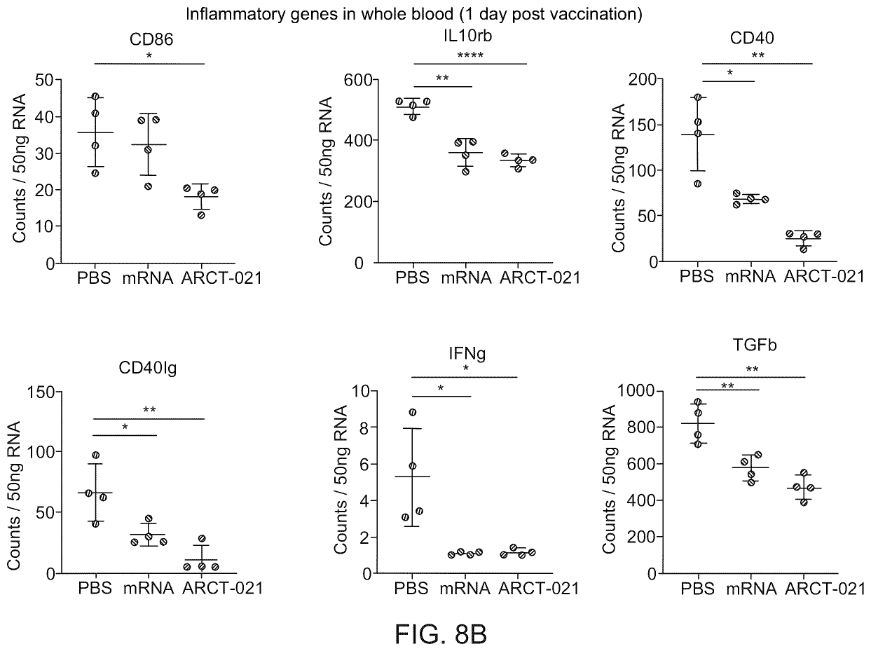

The innate immune response, such as the type-I interferon (IFN) response, has previously been shown to be associated with vaccine immunogenicity following yellow fever vaccination, for example. Furthermore, reactive oxygen species-driven pro-inflammatory responses have been shown to underpin systemic adverse events in yellow fever vaccination. Therefore, the expression of innate immune and pro-inflammatory genes in whole blood of C57BL/6 mice inoculated was measured with either PBS, mRNA vaccine, or the STARR™ SARS-CoV-2 RNA construct. Genes in the type-I IFN pathway were the most highly expressed in animals inoculated with STARR™ SARS-CoV-2 RNA compared to either mRNA vaccine or PBS (FIG. 2B and FIG. 8). By contrast, genes associated with pro-inflammatory responses were mostly reduced in abundance following vaccination STARR™ SARS-CoV-2 RNA compared with either mRNA vaccine or PBS (FIG. 2B and FIG. 8).

Since adaptive immune responses develop in germinal centers in the draining lymph nodes, the draining lymph nodes were dissected at day 7 post-inoculation (study schematic in FIG. 2A). The inguinal lymph nodes of mice inoculated with STARR™ SARS-CoV-2 RNA showed a dose-dependent increase in weight, unlike those from mice inoculated with either mRNA vaccine or PBS; the mean weight of lymph nodes from mice given 10 μg of STARR™ SARS-CoV-2 RNA was significantly higher than those given the equivalent mRNA vaccine (FIG. 2C). Principal component analysis (PCA) of immune gene expression showed clustering of responses to each of the 3 doses of STARR™ SARS-CoV-2 RNA away from the PBS control (STARR™ RNA): depicted as lower sphere in FIG. 2D, smallest sphere in FIG. 2E, and lower sphere in FIG. 2F; PBS control: depicted as upper sphere in FIG. 2D, lower elongated and narrow sphere in FIG. 2E, and upper sphere in FIG. 2F) indicating clear differences in immune gene expression between STARR™ SARS-CoV-2 RNA vaccinated and placebo groups. These trends were also dissimilar to those from mice given mRNA vaccine where at all tested doses, the PCA displayed substantial overlap with placebo (mRNA: shown as center sphere in FIG. 2D, large upright sphere in FIG. 2E, and as flat line with four data points along the bottom of the center square in FIG. 2F; placebo (PBS control): shown as upper sphere in FIG. 2D, lower elongated and narrow sphere in FIG. 2E, and upper sphere in FIG. 2F).

Differentially expressed genes in the lymph nodes of mice given STARR™ SARS-CoV-2 RNA compared to those inoculated with mRNA vaccine were assessed next. Volcano plot analysis identified significant upregulation of several innate, B cell, and T cells genes in STARR™ SARS-CoV-2 RNA immunized animals (FIG. 2G-2I). Some of the most highly differentially expressed genes included, for example, GZMB (important for target cell killing by cytotoxic immune cells), S100A8 and S100A9 (factors that regulate immune responses via TLR4), TNFRSF17 (also known as BCMA and regulates humoral immunity), CXCR3 (chemokine receptor involved in T cell trafficking and function) and AICDA (mediates antibody class switching and somatic hypermutation in B cells).

These findings collectively indicate that the adaptive immune responses in the draining lymph nodes of mice inoculated with STARR™ SARS-CoV-2 RNA appeared to be significantly different compared to immune responses in mice inoculated with a non-replicating mRNA vaccine.

### Example 3

This example describes STARR™ SARS-CoV-2 RNA-induced T cell responses.

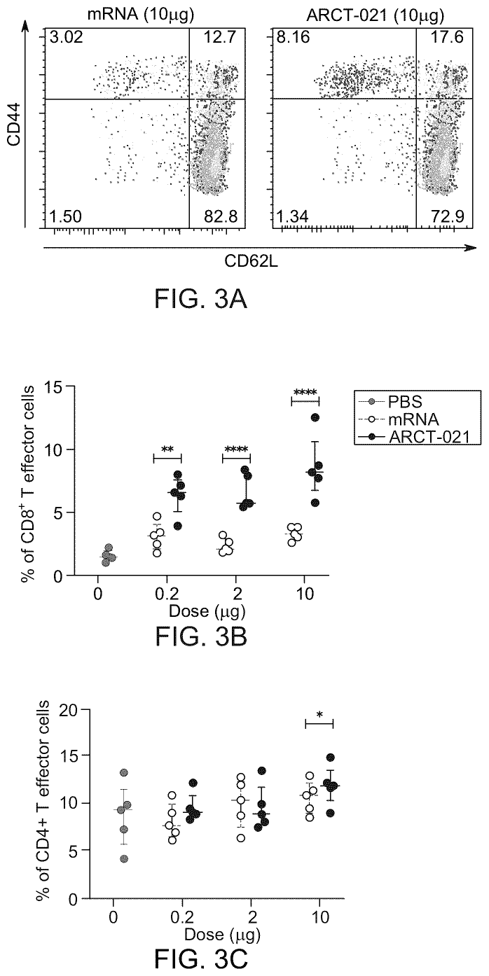

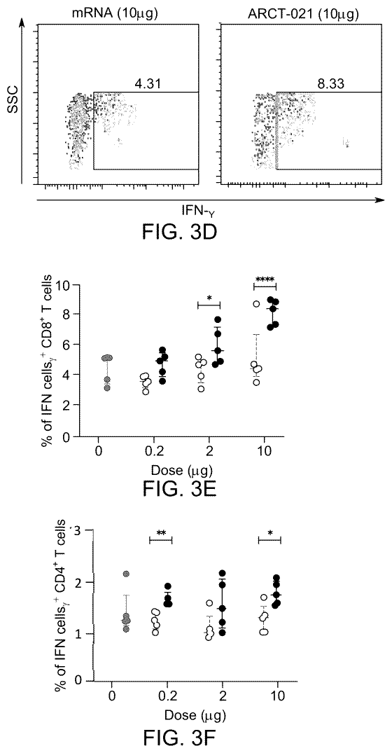

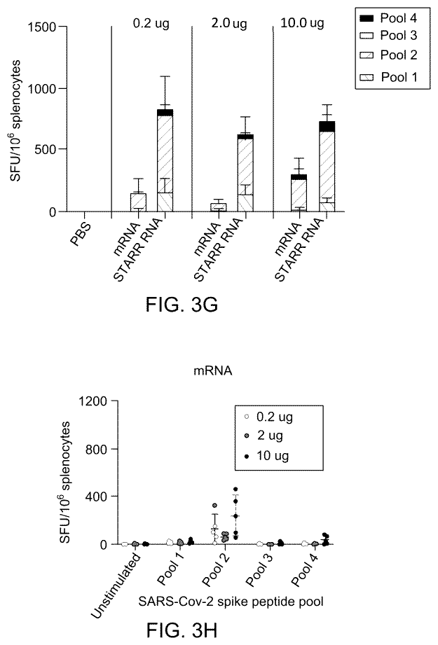

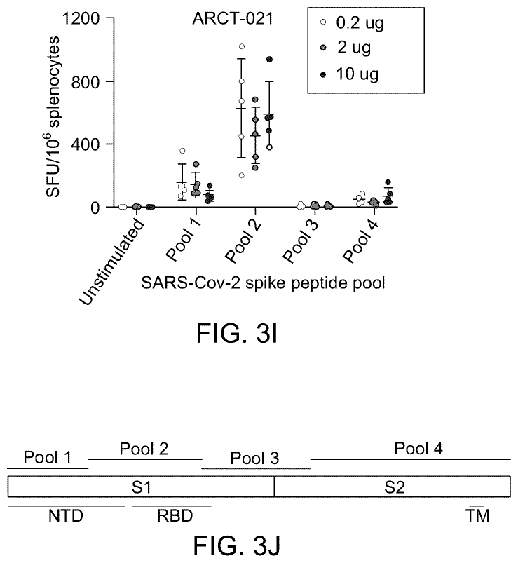

The cellular immune response following vaccination of C57BL/6 mice (n=5 per group) with mRNA or the STARR™ SARS-CoV-2 RNA construct encoding the SARS-CoV-2 glycoprotein described above (Example 1) was investigated next. At day 7 post-vaccination, spleens were harvested and assessed for CD8 and CD4 T cells by flow-cytometry. The CD8+ T cell CD44+CD62L− effector/memory subset was significantly expanded in STARR™ SARS-CoV-2 RNA vaccinated mice compared to those given either PBS or mRNA vaccine (FIG. 3A-B). There was no statistically significant difference in the proportion of CD4+T effector cells of these animals (FIG. 3C). IFNγ+CD8+ T cells (with 2 μg and 10 μg doses) and IFNγ+CD4+ T cells (in 0.2 μg and 10 μg) were proportionately higher, as found using intracellular staining (ICS) with flow cytometry, in STARR™ SARS-CoV-2 RNA as compared to mRNA vaccinated animals (FIG. 3D-3F).

SARS-CoV-2 specific cellular responses were assessed in vaccinated animals by ELISPOT. A set of 15-mer peptides covering the SARS-CoV-2 S protein were divided into 4 pools and tested for IFNγ+ responses in splenocytes of vaccinated and non-vaccinated animals. SARS-CoV-2-specific cellular responses (displayed as IFNγ+SFU/106 cells) were detected by ELISPOT in both STARR™ SARS-CoV-2 RNA and mRNA vaccine immunized animals compared to PBS control (FIG. 3G-3I). These responses were higher across the doses in STARR™ SARS-CoV-2 RNA compared to mRNA vaccinated groups (FIG. 3G-3I). Even the highest tested dose (10 μg) of mRNA vaccine produced IFNγ+ELISPOT responses that were appreciably lower than those by the lowest dose (0.2 μg) of STARR™ SARS-CoV-2 RNA.

These results show that the STARR™ SARS-CoV-2 RNA construct induced strong T cell responses.

### Example 4

This example illustrates humoral responses following vaccination with STARR™ SARS-CoV-2 RNA.

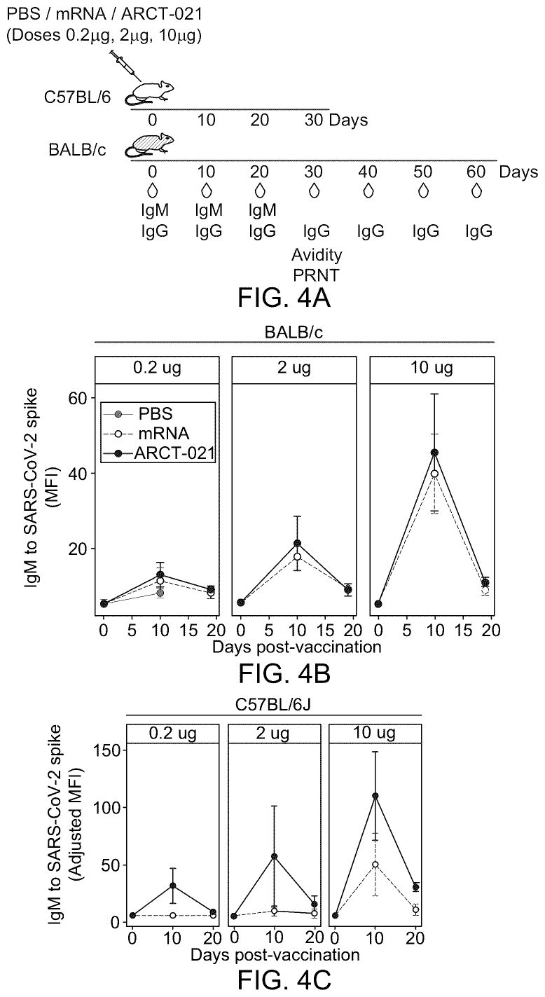

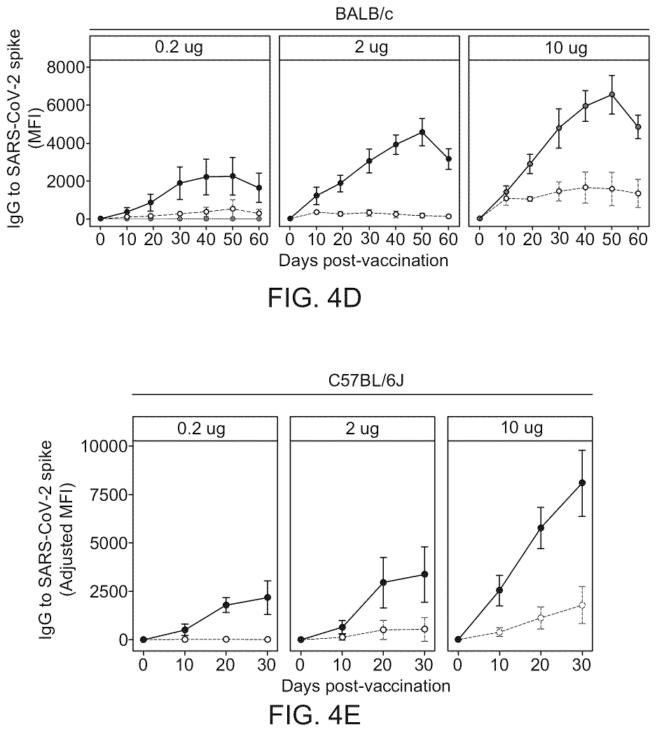

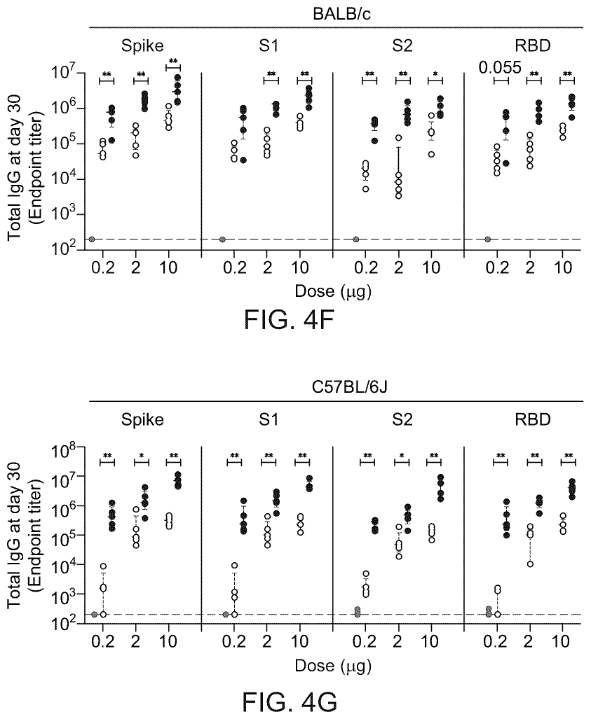

SARS-CoV-2-specific humoral responses following vaccination were characterized in two different mouse models, BALB/c and C57BL/6. Female mice (n=5 per group) were vaccinated at day 0 and bled every 10 days, up to day 60 for BALB/c and day 30 for C57BL/6 (FIG. 4A). SARS-CoV-2 S-specific IgM responses were tested at 1:2000 serum dilution using an in-house Luminex immuno-assay. All tested doses of mRNA vaccine and STARR™ SARS-CoV-2 RNA (corresponding to SEQ ID NO:125, as described in Example 1 above) produced detectable S-specific IgM responses in both mouse models (FIG. 4B-4C). When comparing mRNA to STARR™ SARS-CoV-2 RNA vaccinated BALC/c mice, no difference in IgM responses was observed; IgM levels in C57BL/6 mice were higher in STARR™ SARS-CoV-2 RNA vaccinated C57BL/6 mice at day 10 post vaccination. In contrast, SARS-CoV-2 S-specific IgG (at 1:2000 serum dilution) levels were higher from day 20 onwards in animals inoculated with STARR™ SARS-CoV-2 RNA compared to mRNA vaccine (FIG. 4D-4E). Remarkably, the IgG levels continued to show an increasing trend in STARR™ SARS-CoV-2 RNA vaccinated mice, both BALB/c and C57BL/6, until day 50 post-vaccination with a single inoculation across all the doses. This trend contrasted with mice that received the mRNA vaccine where in BALB/c mice antibody levels plateaued after day 10 post-vaccination; increasing S-specific IgG levels were observed in mRNA-vaccinated C57BL/6 mice but these were lower than those seen in mice that received STARR™ SARS-CoV-2 RNA.

Further characterization of the SARS-CoV-2 specific IgG response in vaccinated animals was conducted at day 30 post-immunization to assess which regions of the S protein are targeted. IgG endpoint titers were estimated to full ectodomain S protein, S1, S2 and receptor binding domain (RBD) regions. For both vaccine candidates the majority of SARS-CoV-2 specific IgG recognized S1, although high IgG endpoint titers were also detected to S2 protein (FIG. 4F-4G). However, STARR™ SARS-CoV-2 RNA elicited IgG endpoint titers were significantly higher compared to those produced by mRNA vaccination (FIG. 4F-4G). Notably, IgG that bind the receptor binding domain (RBD) of S protein, which is an immunodominant site of neutralizing antibodies, were also higher in STARR™ SARS-CoV-2 RNA compared to mRNA vaccinated animals. Furthermore, at lower doses, mRNA vaccine but not STARR™ SARS-CoV-2 RNA struggled to elicit high SARS-CoV-2 specific IgG titers in the more Th1 dominant C57BL/6 mouse strain (FIG. 4G). Taken collectively, a single dose of STARR™ SARS-CoV-2 RNA induced significant differences in immune gene expression and superior cellular immune responses in draining lymph nodes compared to mRNA vaccine and consequently humoral immune responses.

These data show that STARR™ SARS-CoV-2 RNA vaccination induced elevated humoral responses as compared to mRNA vaccination.

### Example 5

This example illustrates reduced risk of immune enhancement upon STARR™ SARS-CoV-2 RNA vaccination.

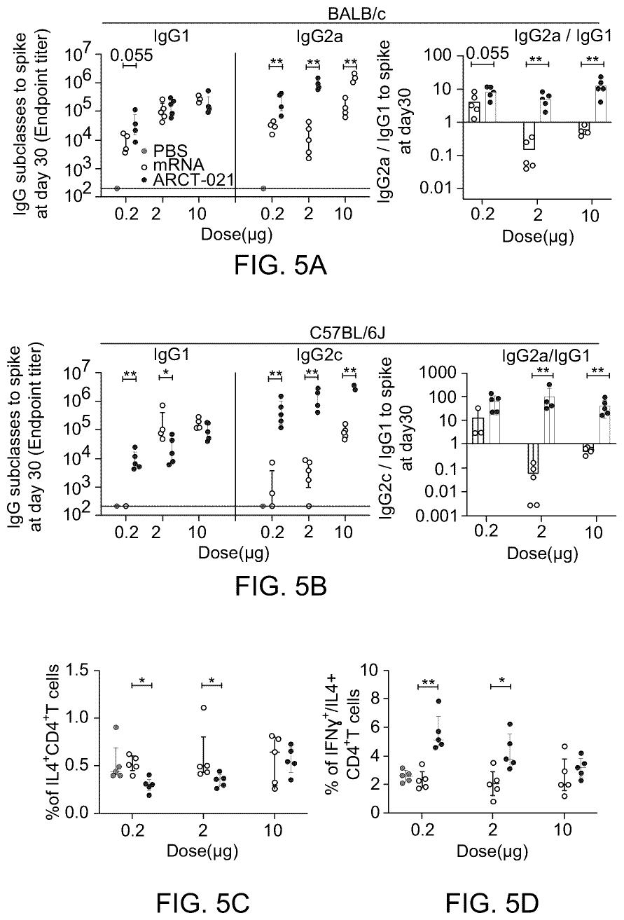

A safety consideration for coronavirus vaccine is a risk of vaccine-mediate immune enhancement of respiratory disease. Indeed, SARS-CoV and MERS-CoV vaccine development have highlighted the importance of Th1 skewed responses to avoid vaccine-induced immune enhancement. Therefore, the Th1/Th2 balance elicited by both mRNA and STARR™ SARS-CoV-2 RNA (self-replicating RNA construct as described in Example 1 above) vaccination was investigated. The IgG subclass fate of plasma cells is influenced by T helper (Th) cells. At day 30 post-vaccination, both mRNA and STARR™ SARS-CoV-2 RNA, except the 0.2 μg dose in C56BL/6J mice, induced comparable amounts of SARS-CoV-2 S-specific IgG1, a Th2-associated IgG subclass in mice (FIG. 5A-5B). In contrast, the Th1-associated IgG subclasses—IgG2a in BALB/c and IgG2c in C56BL/6J—were significantly greater in STARR™ SARS-CoV-2 RNA vaccinated animals. The ratios of S protein-specific IgG2a/IgG1 (BALBc) and IgG2c/IgG1 (C57BL/6) were greater than 1 in STARR™ SARS-CoV-2 RNA vaccinated animals (FIG. 5A-5B). Except for the 0.2 ug dose, these ratios were all significantly greater with STARR™ SARS-CoV-2 RNA compared to mRNA vaccinated animals.

ICS was used to investigate the production of IFNγ (Th1 cytokine) and IL4 (Th2 cytokine) by CD4+ T cells in spleens of day 7 vaccinated C56BL/6J mice. As shown above (Example 3), compared to mRNA vaccination, IFNγ levels were significantly greater in STARR™ SARS-CoV-2 RNA vaccinated animals (FIG. 3F). IL4 expression in CD4 T cells was slightly higher in mRNA as compared to STARR™ SARS-CoV-2 RNA at 0.2 μg and 2 μg doses (FIG. 5C). In comparing the IFNγ and IL4 levels in individual mice, the ratios of IFNγ/IL4 in CD4+ T cells for both STARR™ SARS-CoV-2 RNA and mRNA vaccinated mice were above 1 (FIG. 5D). The ratio of IFNγ/IL4 in CD4+ T cells in mice given the 0.2 μg and 2 μg doses were significantly greater with STARR™ SARS-CoV-2 RNA than mRNA vaccination (FIG. 5F). However, without being limited by theory, the elevated ratios in these doses appeared to be due to the lowered IL4 expression at levels below background (i.e., PBS control mice), rather than reduced IFNγ and hence Th1 activity.

Taken collectively, these data show that STARR™ SARS-CoV-2 RNA produced Th1 instead of Th2 skewed adaptive immune responses.

### Example 6

This example illustrates the quality of STARR™ SARS-CoV-2 RNA-induced humoral immune responses.

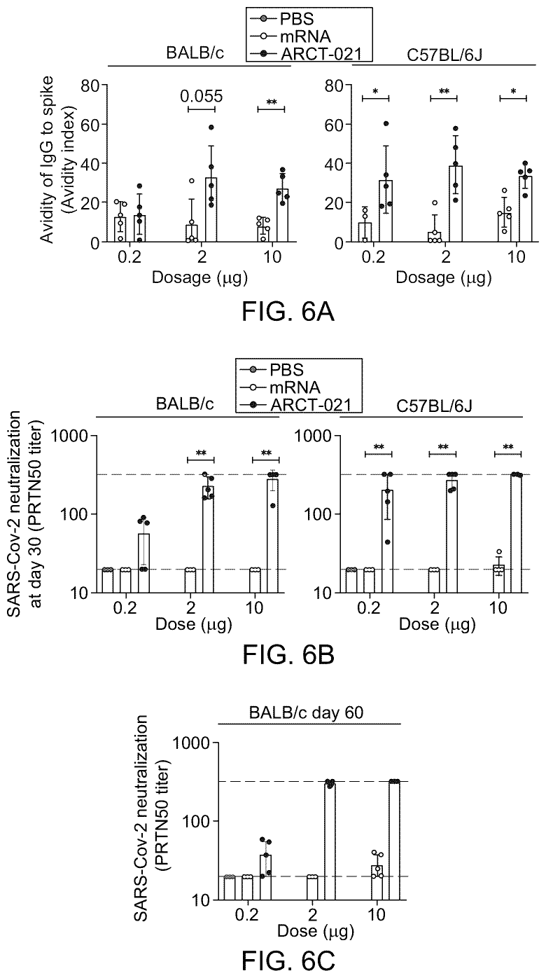

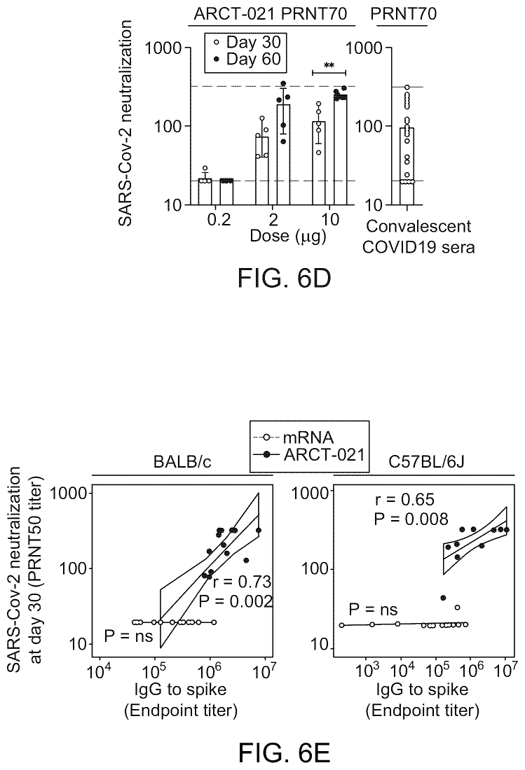

The binding strength (avidity) and the neutralizing ability of the antibody response elicited by the self-replicating STARR™ SARS-CoV-2 RNA (construct as described in Example 1 above) and mRNA vaccine constructs was assessed next. Serum IgG avidity was measured at day 30 post-vaccination using a modified Luminex immuno-assay with 8M urea washes. STARR™ SARS-CoV-2 RNA elicited higher avidity S protein-specific IgG than mRNA in both mouse models at all tested doses (FIG. 6A). These differences were observed, with the exception of 0.2 μg in BALB/c, across all doses (FIG. 6A), indicating that STARR™ SARS-CoV-2 RNA elicited better quality antibodies than conventional mRNA.

Neutralization of live SARS-CoV-2 by serum from vaccinated animals was assessed using the plaque reduction neutralization test (PRNT). At day 30 STARR™ SARS-CoV-2 RNA vaccinated BALB/c showed a clear dose dependent elevation in PRNT50 titers; 4 out of 5 (80%) of mice in the 10 μg STARR™ SARS-CoV-2 RNA group showed PRNT50 titers above the 320 upper limit (FIG. 6B). Similar dose-dependent trends in PRNT50 titers were also found in C57BL/6 mice, although in these animals, the PRNT50 titers of several animals exceeded the 320 upper limit even with a low 0.2 μg dose vaccination (FIG. 6B). In contrast, PRNT50 titers in animals inoculated with mRNA vaccine construct were, except for one C57BL/6J mouse that received 10 μg dose, all <20 (FIG. 6B). Unexpectedly and surprisingly, PRNT50 and PRNT70 titers of STARR™ SARS-CoV-2 RNA vaccinated BALBc mice continued to rise between day 30 and day 60 after a single dose of vaccination (FIG. 6C-6D). These titers were also comparable to PRNT70 titers in sera from convalescent COVID-19 patients (FIG. 6D).

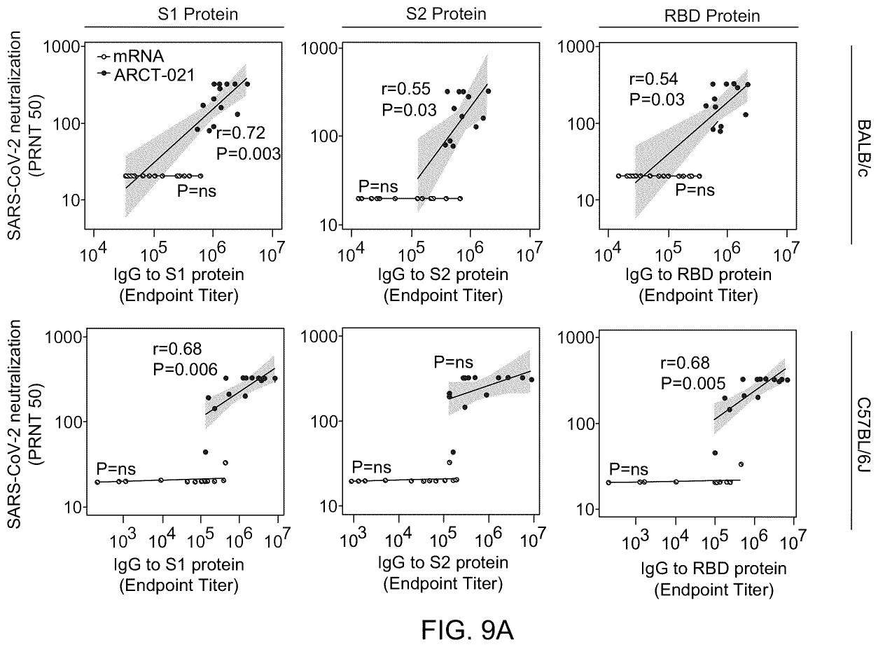

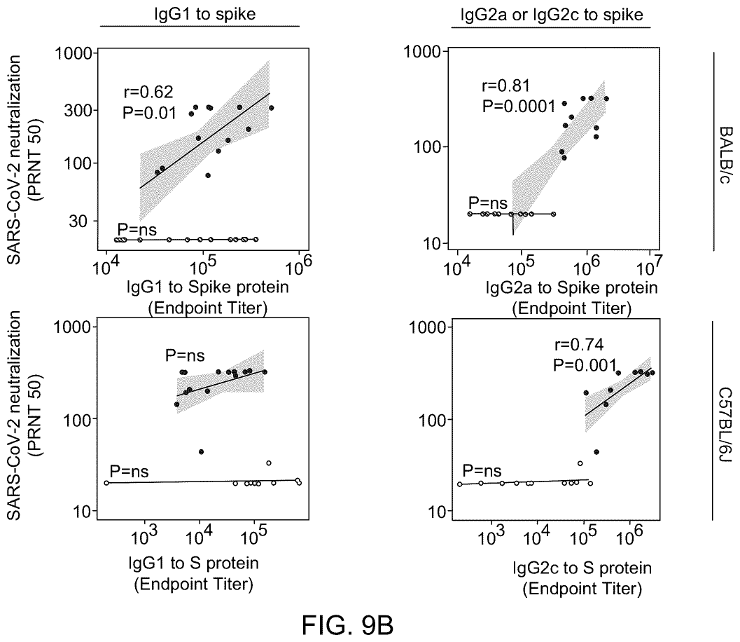

S protein IgG titers also positively correlated with PRNT50 titers in both mouse models (FIG. 6E). Similar positive correlations were also observed with IgG against S1 and RBD (FIG. 9). By contrast, no correlation was found between IgG and PRNT50 titers in mRNA vaccinated mice (FIG. 6E). Taken collectively, without being limited by theory, these antibody response analyses indicate that the higher PRNT50 titers following STARR™ SARS-CoV-2 RNA vaccination are not only attributable to the amount of IgG produced but also due to superiority of the quality of the anti-SARS-CoV-2 antibodies.

In summary, STARR™ SARS-CoV-2 RNA induced qualitatively superior humoral immune response than conventional mRNA.

### Example 7

This example illustrates the effect of a second dose of STARR™ SARS-CoV-2 RNA.

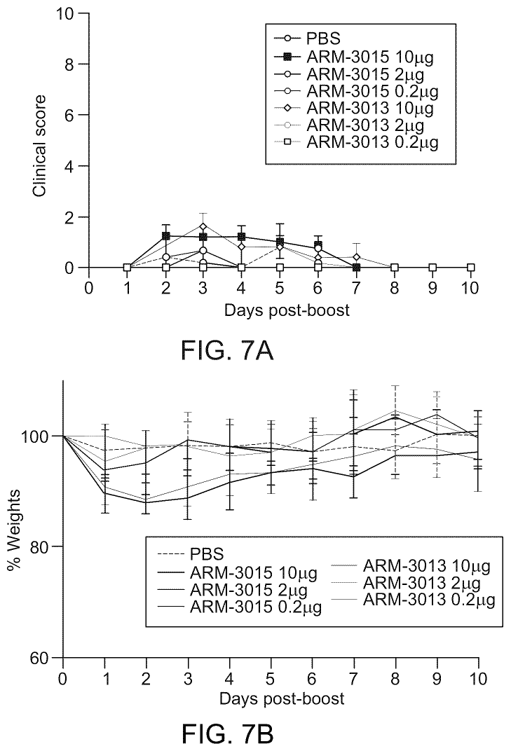

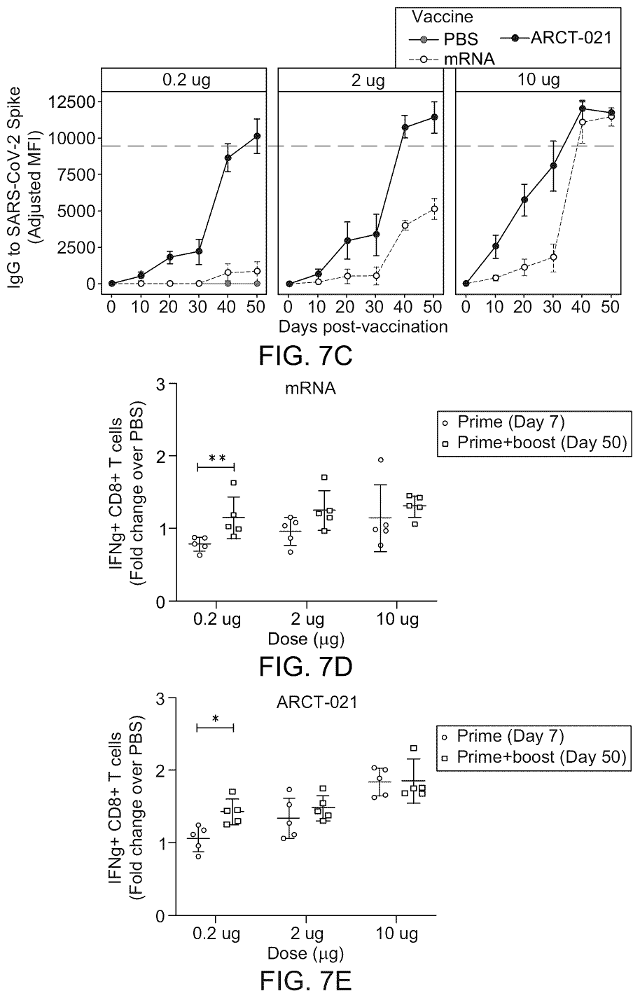

A possible added benefit of a second dose of STARR™ SARS-CoV-2 RNA (self-replicating RNA construct as described in Example 1 above) to the cellular and humoral immune responses to the S protein of SARS-CoV-2 was explored next. The clinical scores after the second dose were higher than after the first dose (FIG. 7A). Like the first dose, mice that received 2 μg and 10 μg of STARR™ SARS-CoV-2 RNA experienced weight loss (FIG. 7B). The IgG response to a second dose of STARR™ SARS-CoV-2 RNA produced an appreciable boost in S protein-specific IgG levels, but only with 0.2 μg and 2 μg of STARR™ SARS-CoV-2 RNA (FIG. 7C). Without being limited by theory, a likely reason for the lack of increase in the anti-S protein specific IgG levels for the 10 μg dose is that the amount of fluorescence is near the saturation point of the detector and sera was not further diluted to observe and increase. However, in a subsequent Balb/c mouse study, the sera from mice vaccinated with a 5 μg RNA dose administered unilaterally in a 0.05 mL injection volume produced a significant increase in neutralizing antibody titers as assayed using a 96 well microneutralization assay format. Mice were bled every 14 days and a second vaccination of 5 μg was administered on day 28. 4 mice were injected with a VEEV replicon RNA expressing luciferase as a negative control and 6 mice were vaccinated with STARR™ SARS-CoV-2 RNA. The results are shown in Table 8 below.

TABLE 7
Microneutralization Titers (MN50) in Balb/c Mice

| Mouse | | Microneutralization Titers (MN50) | | | | |
|---|---|---|---|---|---|---|
| No. | Treatment | Wk 0 | Wk2 | Wk4 | Wk6 | Wk8 |
| 1 | Luciferase | <10 | <10 | <10 | <10 | <10 |
| 2 | Luciferase | <10 | <10 | <10 | <10 | <10 |
| 3 | Luciferase | <10 | <10 | <10 | <10 | <10 |
| 4 | Luciferase | <10 | <10 | <10 | <10 | <10 |
| 5 | STARR ™ SARS- | <10 | 1,280 | 5,120 | 327,680 | 81,920 |
| | CoV-2 RNA | | | | | |
| 6 | STARR ™ SARS- | <10 | 640 | 20,480 | 327,680 | 327,680 |
| | CoV-2 RNA | | | | | |
| 7 | STARR ™ SARS- | <10 | 1,280 | 2,560 | 163,840 | 163,840 |
| | CoV-2 RNA | | | | | |
| 8 | STARR ™ SARS- | <10 | 1,280 | 10,240 | 327,680 | 163,840 |
| | CoV-2 RNA | | | | | |
| 9 | STARR ™ SARS- | <10 | 640 | 40,960 | 327,680 | 327,680 |
| | COV-2 RNA | | | | | |
| 10 | STARR ™ SARS- | <10 | 1,280 | 10,240 | 327,680 | 327,680 |
| | CoV-2 RNA | | | | | |
| Avg | | | 1,016 | 10,240 | 29,1930 | 206,426 |
| Geometric | | | | | | |
| Mean | | | | | | |

The neutralization titers increased ˜10 fold between day 14 an day 28 post vaccination. Following the boost on day 28, the neutralization titers increased an additional 20 fold 14 days post boost.

To determine if there was added benefit in IFNγ+CD8+ T cell counts from a second dose vaccination, CD8 T cell IFNγ responses in mice given only a prime were compared to responses of mice given a prime and a boost. Fold change in IFNγ+CD8+ T cells in the vaccinated over PBS control mice was calculated for mice given either a prime only or given a prime and boost. The fold change of IFNγ+CD8+ T cells was similar following the prime and prime+boost for 2 μg and 10 μg doses of STARR™ SARS-CoV-2 RNA (FIG. 7D-7E); the 0.2 μg dose showed higher fold change of IFNγ+CD8+ T cells between prime (at day 7) and prime+boost (day 50). Vaccination with 0.2 μg of mRNA also showed increased IFNγ+CD8+ T cells relative to PBS control after two doses of vaccination. Without being limited by theory, these findings suggest that a second 10 μg dose of STARR™ SARS-CoV-2 RNA did not produce superior cellular immunity compared to single dose vaccination. Thus, there was no apparent benefit from a second 10 μg dose of STARR™ SARS-CoV-2 RNA.

Taken collectively, these data suggest that 10 μg STARR™ SARS-CoV-2 RNA offers the opportunity of a single dose vaccination to protect against COVID-19.

### Example 8

This example illustrates protection from SARS-CoV-2 viral challenge in mice following vaccination with STARR™ SARS-CoV-2 self-replicating RNA.

A mouse viral challenge study was conducted with human ACE2 transgenic mice. Mice were immunized with 2 μg and 10 μg RNA doses of STARR™ SARS-CoV-2 RNA (RNA construct as described in Example 1 above) or injected with PBS. There were three different cohorts with 5 mice in each treatment group. Cohorts 1 and 3 received a lethal SARS-CoV-2 virus challenge load of 5×105 TCID50. Cohort 1 was monitored for survival and Cohort 3 was euthanized 5 days after challenge. Lungs were assayed for viral load and processed for histopathology. Cohorts 2 received a sublethal viral load of 5×104 TCID50. Cohort 2 was euthanized 5 days after virus challenge and lungs were assayed for infectious virus and processed for histopathology. All mice were inoculated intratracheally 30 days post-vaccination with a single dose of STARR™ SARS-CoV-2 RNA.

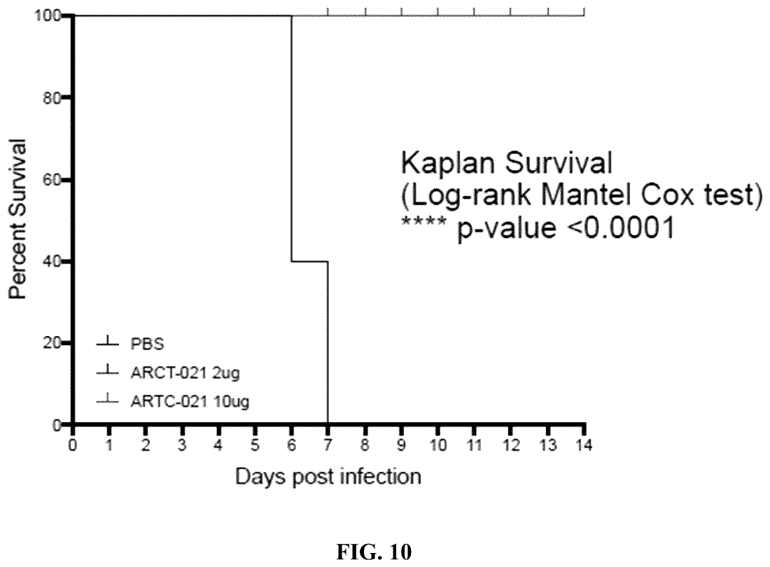

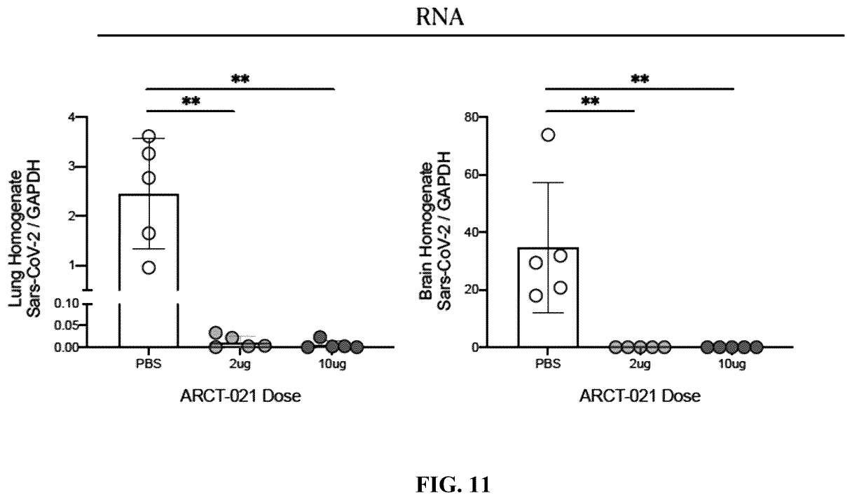

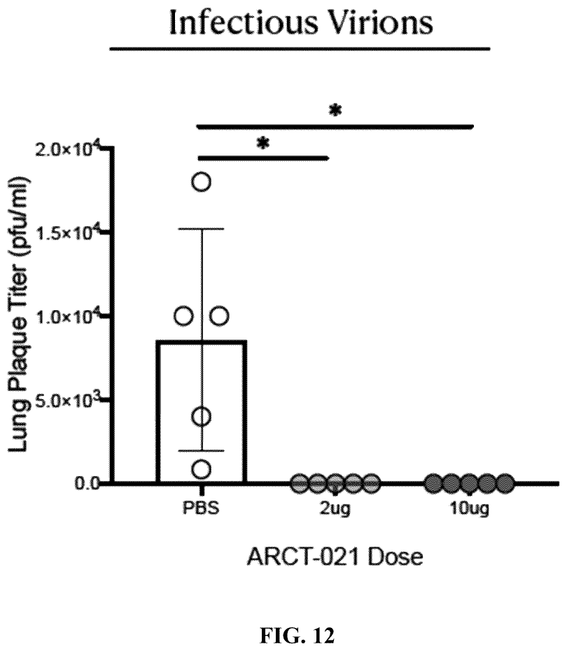

All mice injected with PBS in cohort 1 were dead by day 7, whereas all vaccinated mice showed no signs of infection 15 days after viral challenge (FIG. 10). For Cohort 2 receiving a sublethal viral load, 10 to 3,300 copies of virus was measured by RT-PCR in the lungs with an average of 1,200 copies, whereas no copies of viral RNA were measured in mice vaccinated with ARTC-021 at 2 μg and 10 μg RNA doses (LOD was 0.1 copies; FIG. 11, left). Copies of viral RNA were also observed in the brain ranging from 20 to 80 in the PBS treatment group, whereas no viral RNA copies were measured in the brains of mice vaccinated with either 2.0 μg or 10.0 μg RNA doses (FIG. 11, right). Lastly, lungs were carefully processed and assayed for lung plaque titers. The average plaque titers for the group injected with PBS was 8×103/mL of lung homogenate, whereas no plaques were detected for mice vaccinated with either 2.0 μg or 10.0 μg or STARR™ SARS-CoV-2 RNA (FIG. 12). Lung and brain tissues from Cohort 3 are being assayed for viral copy number and infectious virus. Histopathology of lungs for cohorts 2 and 3 is in progress.

These results show that vaccination with STARR™ SARS-CoV-2 self-replicating RNA protected mice from a lethal SARS-CoV-2 infection and protected against lung and brain infection upon challenge with a sublethal dose of SARS-CoV-2.

### Example 9

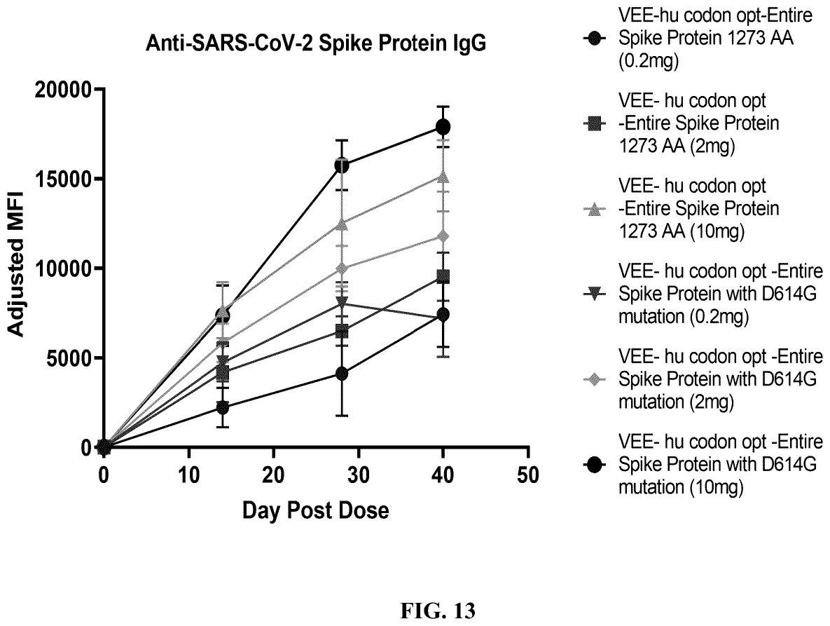

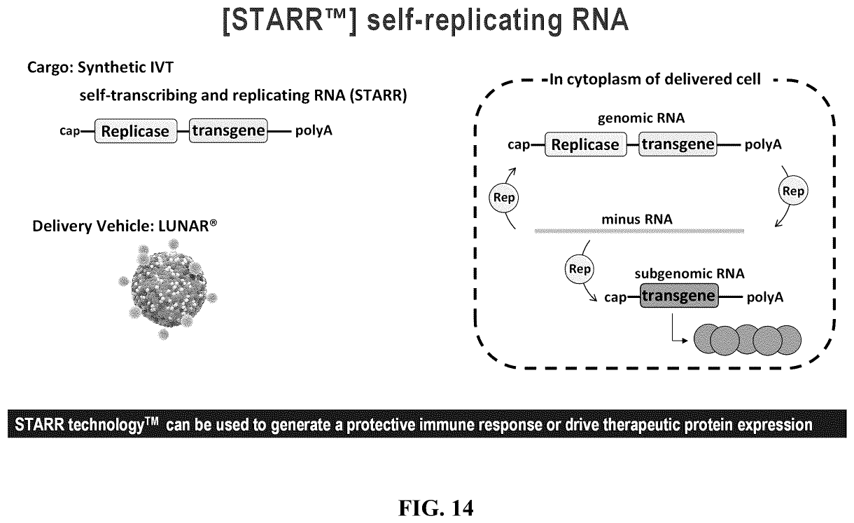

The COVID-19 pandemic is caused by infection with the SARS-CoV-2 virus. A major mutation detected to date in the SARS-CoV-2 viral envelope spike protein, which is responsible for virus attachment to the host and is also the main target for host antibodies, is a mutation of an aspartate (D) at position 614 found frequently in Chinese strains to a glycine (G). VEEV Replicon transcripts expressing the D614 and G614 versions of the SARS-CoV-2 spike glycoprotein were formulated with the exact same lipid formulation as studies described in Examples 1-8. Balb/c mice were vaccinated with a single RNA administration of 0.2 μg, 2.0 μg and 10.0 μg of RNA. There were 5 mice per dose. Mice were bled on days, 14, 28 and 42 post vaccination. Sera was diluted 1/2000 and incubated with Luminex beads derivatized with the SARS-CoV-2 spike glycoprotein containing the D614 amino acid sequence. A secondary mouse antibody derivatized with a fluorophore was used to assay for bound antibody to the beads and adjusted mean fluorescence intensity (MFI) was measured as a function of RNA dose, shown in FIG. 13. The results showed that MFI increased as a function of RNA dose with slightly higher MFI observed for the serum from mice immunized with the G614 spike glycoprotein. This slight elevation is attributed to a lower percentage of full length RNA with the D614 amino acid sequence. An important conclusion is that the serum from mice immunized with the G614 spike glycoprotein RNA construct was able to bind to spike glycoprotein with the D614 amino acid sequence, indicative of cross reactivity.

These results show that immunization with a G614 spike glycoprotein expressed from self-replicating RNA results in production of antibodies that are able to bind to a D614 spike glycoprotein.

### Discussion of Examples 1-9

The pandemic of COVID-19 has necessitated rapid development of vaccines, as physical distancing to prevent SARS-CoV-2 transmission is not a sustainable long-term solution. Several COVID-19 vaccine candidates are now in clinical trials and more are entering first-in-human trials. However, a majority of vaccine candidates being developed require two doses for sufficient adaptive immunity. A single dose vaccine that generates both cellular and humoral immunity, without elevating the risk of vaccine-mediated immune enhancement, remains an unmet need. Without being limited by theory, deployment of a single dose vaccine would enable greater level of compliance and enable distribution of finite production of vaccines to more susceptible people globally.

Among licensed vaccines, live attenuated vaccines can offer durable protection against viral diseases. Live vaccines infect and replicate at sites of inoculation and some even in draining lymph nodes. Replication enables endogenous expression of viral antigens that enables antigen presentation to stimulate cytotoxic CD8+ T cells. Expressed antigens would also be taken up by antigen presenting cells to trigger CD4+ T cell help that drive affinity maturation in B cells. Studies on the live attenuated yellow fever vaccine have shown that a longer period of stimulation of the adaptive immune response results in superior adaptive immune responses. Without being limited by theory, simulating the processes of live vaccination could offer a chance of durable immunity against COVID-19.

In a crisis such as COVID-19, a nucleic acid vaccine platform offers opportunities for accelerated development. In studies described herein, a side-by-side comparison of the immunogenicity elicited by two SARS-CoV-2 vaccines candidates was conducted, a non-replicative mRNA construct and STARR™ SARS-CoV-2 RNA. Compared to an mRNA vaccine, STARR™ SARS-CoV-2 RNA produced higher and longer protein expression in vivo and upregulated gene expression of several innate, B cell, and T cell response genes in the blood and draining lymph nodes. These properties translated into significantly greater CD8+ T cell responses, IFNγ+ELISPOT responses, and SARS-CoV-2 specific IgG and Th1 skewed responses. Interestingly, despite the highest tested dose of mRNA eliciting comparable S protein-specific antibodies as the lowest tested dose of STARR™ SARS-CoV-2 RNA, mRNA-elicited IgG did not show similar avidity or neutralization activity as those from STARR™ SARS-CoV-2 RNA vaccination. These findings thus highlight the immunological advantages of self-replicating RNA over mRNA platforms. In addition, mouse challenge studies with SARS-CoV-2 virus showed that vaccination with a single high dose (10 μg) or a single low dose (2 μg) of STARR™ SARS-CoV-2 self-replicating RNA protected mice from a lethal SARS-CoV-2 infection and protected from lung and brain infection upon challenge with a sublethal SARS-CoV-2 dose.

The extent to which STARR™ vaccines reproduce the features of live vaccines remain to be experimentally defined. Without being limited by theory, the superior quality of immune responses elicited by STARR™ SARS-CoV-2 RNA over the mRNA vaccine construct could be attributable to multiple factors, all of which have been found to be associated with live vaccination. For example, higher and longer expression of immunogens produce better immunity, likely through better engagement of T follicular helper cells and thereby leading to more diverse antibody targets and more neutralizing antibody responses. Replication of STARR™ SARS-CoV-2 RNA would result in the formation of a negative-strand template for production of more positive-strand mRNA and sub-genomic mRNA expressing the S transgene. Interaction between the negative- and positive-strands would form double stranded RNA (dsRNA), which would interact with TLR3 and RIG-I-like receptors to stimulate interferon responses, which has been shown to correlate with superior adaptive immune responses. Production of IFNγ can then stimulate development of cytotoxic CD8+ T cells. Importantly, the S protein does contain human CD8+ T cell epitopes. Without being limited by theory, the development of T cell memory could be important for long-term immunity, as suggested by recent findings on T cell responses to SARS-CoV-2 and other coronavirus infections.

It is unclear whether the VEEV nsP1-4 forming the replication complex contains any immunogenic properties, although mutations in the nsP proteins have been shown to affect induction of type I IFN. VEEV replicons have also been shown to adjuvant immune responses at mucosal sites, further illustrating the advantages of using the STARR™ platform to develop a COVID-19 vaccine. Without being limited by theory, there does not appear to be an immune response to replicon non-structural proteins, as indicated by an increase in antigen-specific IgG production upon a second administration of replicon to animals. In the presence of an immune response to non-structural proteins, a limited or no increase in antigen-specific IgG production may have resulted following a second administration. The RNA is encapsulated in lipid nanoparticles (LNP), which together can form potent adjuvants leading to robust immune responses. In addition, using the genetic sequence of an antigen, including a viral antigen such as the spike protein from SARS-CoV-2, for example, STARR™ vaccines can be rapidly generated and manufactured using cell-free and rapidly scalable techniques.

In conclusion, a STARR™ vaccine as exemplified by STARR™ SARS-CoV-2 RNA offers an approach to simulate several of the properties of live vaccination and offers a potential for single-dose vaccination against COVID-19.

SEQUENCES
SEQ ID NO: 72
ATGGAGAAAGTTCACGTTGACATCGAGGAAGACAGCCCATTCCTCAGAGCTTTG
CAGCGGAGCTTCCCGCAGTTTGAGGTAGAAGCCAAGCAGGTCACTGATAATGAC
CATGCTAATGCCAGAGCGTTTTCGCATCTGGCTTCAAAACTGATCGAAACGGAGG
TGGACCCATCCGACACGATCCTTGACATTGGAAGTGCGCCCGCCCGCAGAATGT
ATTCTAAGCACAAGTATCATTGTATCTGTCCGATGAGATGTGCGGAAGATCCGGA
CAGATTGTATAAGTATGCAACTAAGCTGAAGAAAAACTGTAAGGAAATAACTGA
TAAGGAATTGGACAAGAAAATGAAGGAGCTGGCCGCCGTCATGAGCGACCCTGA
CCTGGAAACTGAGACTATGTGCCTCCACGACGACGAGTCGTGTCGCTACGAAGG
GCAAGTCGCTGTTTACCAGGATGTATACGCCGTCGACGGCCCCACCAGCCTGTAC
CACCAGGCCAACAAGGGCGTGAGGGTGGCCTACTGGATCGGCTTCGACACCACA
CCCTTCATGTTCAAGAACCTGGCCGGCGCCTACCCCAGCTACAGCACCAACTGGG
CCGACGAGACCGTGCTGACCGCCAGGAACATCGGCCTGTGCAGCAGCGACGTGA
TGGAGAGGAGCCGGAGAGGCATGAGCATCCTGAGGAAGAAATACCTGAAGCCC
AGCAACAACGTGCTGTTCAGCGTGGGCAGCACCATCTACCACGAGAAGAGGGAC
CTGCTCAGGAGCTGGCACCTGCCCAGCGTGTTCCACCTGAGGGGCAAGCAGAAC
TACACCTGCAGGTGCGAGACCATCGTGAGCTGCGACGGCTACGTGGTGAAGAGG
ATCGCCATCAGCCCCGGCCTGTACGGCAAGCCCAGCGGCTACGCCGCTACAATG
CACAGGGAGGGCTTCCTGTGCTGCAAGGTGACCGACACCCTGAACGGCGAGAGG
GTGAGCTTCCCCGTGTGCACCTACGTGCCCGCCACCCTGTGCGACCAGATGACCG
GCATCCTGGCCACCGACGTGAGCGCCGACGACGCCCAGAAGCTGCTCGTGGGCC
TGAACCAGAGGATCGTGGTCAACGGCAGGACCCAGAGGAACACCAACACAATG
AAGAACTACCTGCTGCCCGTGGTGGCCCAGGCTTTCGCCAGGTGGGCCAAGGAG
TACAAGGAGGACCAGGAAGACGAGAGGCCCCTGGGCCTGAGGGACAGGCAGCT
GGTGATGGGCTGCTGCTGGGCCTTCAGGCGGCACAAGATCACCAGCATCTACAA
GAGGCCCGACACCCAGACCATCATCAAGGTGAACAGCGACTTCCACAGCTTCGT
GCTGCCCAGGATCGGCAGCAACACCCTGGAGATCGGCCTGAGGACCCGGATCAG
GAAGATGCTGGAGGAACACAAGGAGCCCAGCCCACTGATCACCGCCGAGGACGT
GCAGGAGGCCAAGTGCGCTGCCGACGAGGCCAAGGAGGTGAGGGAGGCCGAGG
AACTGAGGGCCGCCCTGCCACCCCTGGCTGCCGACGTGGAGGAACCCACCCTGG
AAGCCGACGTGGACCTGATGCTGCAGGAGGCCGGCGCCGGAAGCGTGGAGACA
CCCAGGGGCCTGATCAAGGTGACCAGCTACGACGGCGAGGACAAGATCGGCAGC
TACGCCGTGCTGAGCCCACAGGCCGTGCTGAAGTCCGAGAAGCTGAGCTGCATC
CACCCACTGGCCGAGCAGGTGATCGTGATCACCCACAGCGGCAGGAAGGGCAGG
TACGCCGTGGAGCCCTACCACGGCAAGGTGGTCGTGCCCGAGGGCCACGCCATC
CCCGTGCAGGACTTCCAGGCCCTGAGCGAGAGCGCCACCATCGTGTACAACGAG
AGGGAGTTCGTGAACAGGTACCTGCACCATATCGCCACCCACGGCGGAGCCCTG
AACACCGACGAGGAATACTACAAGACCGTGAAGCCCAGCGAGCACGACGGCGA
GTACCTGTACGACATCGACAGGAAGCAGTGCGTGAAGAAAGAGCTGGTGACCGG
CCTGGGACTGACCGGCGAGCTGGTGGACCCACCCTTCCACGAGTTCGCCTACGA
GAGCCTGAGGACCAGACCCGCCGCTCCCTACCAGGTGCCCACCATCGGCGTGTA
CGGCGTGCCCGGCAGCGGAAAGAGCGGCATCATCAAGAGCGCCGTGACCAAGA
AAGACCTGGTGGTCAGCGCCAAGAAAGAGAACTGCGCCGAGATCATCAGGGAC
GTGAAGAAGATGAAAGGCCTGGACGTGAACGCGCGCACCGTGGACAGCGTGCTG
CTGAACGGCTGCAAGCACCCCGTGGAGACCCTGTACATCGACGAGGCCTTCGCTT
GCCACGCCGGCACCCTGAGGGCCCTGATCGCCATCATCAGGCCCAAGAAAGCCG
TGCTGTGCGGCGACCCCAAGCAGTGCGGCTTCTTCAACATGATGTGCCTGAAGGT
GCACTTCAACCACGAGATCTGCACCCAGGTGTTCCACAAGAGCATCAGCAGGCG
GTGCACCAAGAGCGTGACCAGCGTCGTGAGCACCCTGTTCTACGACAAGAAAAT
GAGGACCACCAACCCCAAGGAGACCAAAATCGTGATCGACACCACAGGCAGCA
CCAAGCCCAAGCAGGACGACCTGATCCTGACCTGCTTCAGGGGCTGGGTGAAGC
AGCTGCAGATCGACTACAAGGGCAACGAGATCATGACCGCCGCTGCCAGCCAGG
GCCTGACCAGGAAGGGCGTGTACGCCGTGAGGTACAAGGTGAACGAGAACCCAC
TGTACGCTCCCACCAGCGAGCACGTGAACGTGCTGCTGACCAGGACCGAGGACA
GGATCGTGTGGAAGACCCTGGCCGGCGACCCCTGGATCAAGACCCTGACCGCCA
AGTACCCCGGCAACTTCACCGCCACCATCGAAGAGTGGCAGGCCGAGCACGACG
CCATCATGAGGCACATCCTGGAGAGGCCCGACCCCACCGACGTGTTCCAGAACA
AGGCCAACGTGTGCTGGGCCAAGGCCCTGGTGCCCGTGCTGAAGACCGCCGGCA
TCGACATGACCACAGAGCAGTGGAACACCGTGGACTACTTCGAGACCGACAAGG
CCCACAGCGCCGAGATCGTGCTGAACCAGCTGTGCGTGAGGTTCTTCGGCCTGGA
CCTGGACAGCGGCCTGTTCAGCGCCCCCACCGTGCCACTGAGCATCAGGAACAA
CCACTGGGACAACAGCCCCAGCCCAAACATGTACGGCCTGAACAAGGAGGTGGT
CAGGCAGCTGAGCAGGCGGTACCCACAGCTGCCCAGGGCCGTGGCCACCGGCAG
GGTGTACGACATGAACACCGGCACCCTGAGGAACTACGACCCCAGGATCAACCT
GGTGCCCGTGAACAGGCGGCTGCCCCACGCCCTGGTGCTGCACCACAACGAGCA
CCCACAGAGCGACTTCAGCTCCTTCGTGAGCAAGCTGAAAGGCAGGACCGTGCT
GGTCGTGGGCGAGAAGCTGAGCGTGCCCGGCAAGATGGTGGACTGGCTGAGCGA
CAGGCCCGAGGCCACCTTCCGGGCCAGGCTGGACCTCGGCATCCCCGGCGACGT
GCCCAAGTACGACATCATCTTCGTGAACGTCAGGACCCCATACAAGTACCACCAT
TACCAGCAGTGCGAGGACCACGCCATCAAGCTGAGCATGCTGACCAAGAAGGCC
TGCCTGCACCTGAACCCCGGAGGCACCTGCGTGAGCATCGGCTACGGCTACGCC
GACAGGGCCAGCGAGAGCATCATTGGCGCCATCGCCAGGCTGTTCAAGTTCAGC
AGGGTGTGCAAACCCAAGAGCAGCCTGGAGGAAACCGAGGTGCTGTTCGTGTTC
ATCGGCTACGACCGGAAGGCCAGGACCCACAACCCCTACAAGCTGAGCAGCACC
CTGACAAACATCTACACCGGCAGCAGGCTGCACGAGGCCGGCTGCGCCCCCAGC
TACCACGTGGTCAGGGGCGATATCGCCACCGCCACCGAGGGCGTGATCATCAAC
GCTGCCAACAGCAAGGGCCAGCCCGGAGGCGGAGTGTGCGGCGCCCTGTACAAG
AAGTTCCCCGAGAGCTTCGACCTGCAGCCCATCGAGGTGGGCAAGGCCAGGCTG
GTGAAGGGCGCCGCTAAGCACATCATCCACGCCGTGGGCCCCAACTTCAACAAG
GTGAGCGAGGTGGAAGGCGACAAGCAGCTGGCCGAAGCCTACGAGAGCATCGC
CAAGATCGTGAACGACAATAACTACAAGAGCGTGGCCATCCCACTGCTCAGCAC
CGGCATCTTCAGCGGCAACAAGGACAGGCTGACCCAGAGCCTGAACCACCTGCT
CACCGCCCTGGACACCACCGATGCCGACGTGGCCATCTACTGCAGGGACAAGAA
GTGGGAGATGACCCTGAAGGAGGCCGTGGCCAGGCGGGAGGCCGTGGAAGAGA
TCTGCATCAGCGACGACTCCAGCGTGACCGAGCCCGACGCCGAGCTGGTGAGGG
TGCACCCCAAGAGCTCCCTGGCCGGCAGGAAGGGCTACAGCACCAGCGACGGCA
AGACCTTCAGCTACCTGGAGGGCACCAAGTTCCACCAGGCCGCTAAGGACATCG
CCGAGATCAACGCTATGTGGCCCGTGGCCACCGAGGCCAACGAGCAGGTGTGCA
TGTACATCCTGGGCGAGAGCATGTCCAGCATCAGGAGCAAGTGCCCCGTGGAGG
AAAGCGAGGCCAGCACACCACCCAGCACCCTGCCCTGCCTGTGCATCCACGCTA
TGACACCCGAGAGGGTGCAGCGGCTGAAGGCCAGCAGGCCCGAGCAGATCACC
GTGTGCAGCTCCTTCCCACTGCCCAAGTACAGGATCACCGGCGTGCAGAAGATCC
AGTGCAGCCAGCCCATCCTGTTCAGCCCAAAGGTGCCCGCCTACATCCACCCCAG
GAAGTACCTGGTGGAGACCCCACCCGTGGACGAGACACCCGAGCCAAGCGCCGA
GAACCAGAGCACCGAGGGCACACCCGAGCAGCCACCCCTGATCACCGAGGACG
AGACAAGGACCCGGACCCCAGAGCCCATCATTATCGAGGAAGAGGAAGAGGAC
AGCATCAGCCTGCTGAGCGACGGCCCCACCCACCAGGTGCTGCAGGTGGAGGCC
GACATCCACGGCCCACCCAGCGTGTCCAGCTCCAGCTGGAGCATCCCACACGCC
AGCGACTTCGACGTGGACAGCCTGAGCATCCTGGACACCCTGGAGGGCGCCAGC
GTGACCTCCGGCGCCACCAGCGCCGAGACCAACAGCTACTTCGCCAAGAGCATG
GAGTTCCTGGCCAGGCCCGTGCCAGCTCCCAGGACCGTGTTCAGGAACCCACCCC
ACCCAGCTCCCAGGACCAGGACCCCAAGCCTGGCTCCCAGCAGGGCCTGCAGCA
GGACCAGCCTGGTGAGCACCCCACCCGGCGTGAACAGGGTGATCACCAGGGAGG
AACTGGAGGCCCTGACACCCAGCAGGACCCCCAGCAGGTCCGTGAGCAGGACTA
GTCTGGTGTCCAACCCACCCGGCGTGAACAGGGTGATCACCAGGGAGGAATTCG
AGGCCTTCGTGGCCCAGCAACAGAGACGGTTCGACGCCGGCGCCTACATCTTCA
GCAGCGACACCGGCCAGGGACACCTGCAGCAAAAGAGCGTGAGGCAGACCGTG
CTGAGCGAGGTGGTGCTGGAGAGGACCGAGCTGGAAATCAGCTACGCCCCCAGG
CTGGACCAGGAGAAGGAGGAACTGCTCAGGAAGAAACTGCAGCTGAACCCCAC
CCCAGCCAACAGGAGCAGGTACCAGAGCAGGAAGGTGGAGAACATGAAGGCCA
TCACCGCCAGGCGGATCCTGCAGGGCCTGGGACACTACCTGAAGGCCGAGGGCA
AGGTGGAGTGCTACAGGACCCTGCACCCCGTGCCACTGTACAGCTCCAGCGTGA
ACAGGGCCTTCTCCAGCCCCAAGGTGGCCGTGGAGGCCTGCAACGCTATGCTGA
AGGAGAACTTCCCCACCGTGGCCAGCTACTGCATCATCCCCGAGTACGACGCCTA
CCTGGACATGGTGGACGGCGCCAGCTGCTGCCTGGACACCGCCAGCTTCTGCCCC
GCCAAGCTGAGGAGCTTCCCCAAGAAACACAGCTACCTGGAGCCCACCATCAGG
AGCGCCGTGCCCAGCGCCATCCAGAACACCCTGCAGAACGTGCTGGCCGCTGCC
ACCAAGAGGAACTGCAACGTGACCCAGATGAGGGAGCTGCCCGTGCTGGACAGC
GCTGCCTTCAACGTGGAGTGCTTCAAGAAATACGCCTGCAACAACGAGTACTGG
GAGACCTTCAAGGAGAACCCCATCAGGCTGACCGAAGAGAACGTGGTGAACTAC
ATCACCAAGCTGAAGGGCCCCAAGGCCGCTGCCCTGTTCGCTAAGACCCACAAC
CTGAACATGCTGCAGGACATCCCAATGGACAGGTTCGTGATGGACCTGAAGAGG
GACGTGAAGGTGACACCCGGCACCAAGCACACCGAGGAGAGGCCCAAGGTGCA
GGTGATCCAGGCCGCTGACCCACTGGCCACCGCCTACCTGTGCGGCATCCACAG
GGAGCTGGTGAGGCGGCTGAACGCCGTGCTGCTGCCCAACATCCACACCCTGTTC
GACATGAGCGCCGAGGACTTCGACGCCATCATCGCCGAGCACTTCCAGCCCGGC
GACTGCGTGCTGGAGACCGACATCGCCAGCTTCGACAAGAGCGAGGATGACGCT
ATGGCCCTGACCGCTCTGATGATCCTGGAGGACCTGGGCGTGGACGCCGAGCTG
CTCACCCTGATCGAGGCTGCCTTCGGCGAGATCAGCTCCATCCACCTGCCCACCA
AGACCAAGTTCAAGTTCGGCGCTATGATGAAAAGCGGAATGTTCCTGACCCTGTT
CGTGAACACCGTGATCAACATTGTGATCGCCAGCAGGGTGCTGCGGGAGAGGCT
GACCGGCAGCCCCTGCGCTGCCTTCATCGGCGACGACAACATCGTGAAGGGCGT
GAAAAGCGACAAGCTGATGGCCGACAGGTGCGCCACCTGGCTGAACATGGAGGT
GAAGATCATCGACGCCGTGGTGGGCGAGAAGGCCCCCTACTTCTGCGGCGGATT
CATCCTGTGCGACAGCGTGACCGGCACCGCCTGCAGGGTGGCCGACCCCCTGAA
GAGGCTGTTCAAGCTGGGCAAGCCACTGGCCGCTGACGATGAGCACGACGATGA
CAGGCGGAGGGCCCTGCACGAGGAAAGCACCAGGTGGAACAGGGTGGGCATCC
TGAGCGAGCTGTGCAAGGCCGTGGAGAGCAGGTACGAGACCGTGGGCACCAGC
ATCATCGTGATGGCTATGACCACACTGGCCAGCTCCGTCAAGAGCTTCTCCTACC
TGAGGGGGGCCCCTATAACTCTCTACGGCTAA
SEQ ID NO: 73
ATGGGCGGCGCATGAGAGAAGCCCAGACCAATTACCTACCCAAA
SEQ ID NO: 74
GATGGGCGGCGCATGAGAGAAGCCCAGACCAATTACCTACCCAAA
SEQ ID NO: 75
GATAGGCGGCGCATGAGAGAAGCCCAGACCAATTACCTACCCAAA
SEQ ID NO: 76
ACTCGAGTATGTTACGTGCAAAGGTGATTGTCACCCCCCGAAAGACCATATTGTG
ACACACCCTCAGTATCACGCCCAAACATTTACAGCCGCGGTGTCAAAAACCGCG
TGGACGTGGTTAACATCCCTGCTGGGAGGATCAGCCGTAATTATTATAATTGGCT
TGGTGCTGGCTACTATTGTGGCCATGTACGTGCTGACCAACCAGAAACATAATTG
AATACAGCAGCAATTGGCAAGCTGCTTACATAGAACTCGCGGCGATTGGCATGC
CGCCTTAAAATTTTTATTTTATTTTTTCTTTTCTTTTCCGAATCGGATTTTGTTTTTA
ATATTTCAAAAAAAAAAAAAAAAAAAAAAAAATCTAGAAAAAAAAAAAAAAAA
AAAAAAAAAAAAAAAAAAAAAAAAAAAAAAAAAAAAAAAAAAAAAAAAAAAA
AAAAAAAAAAAAAAAAAAAAAAAAAAAAAAAA
SEQ ID NO: 121
ATGTTTGTTTTTCTTGTTTTATTGCCACTAGTCTCTAGTCAGTGTGTTAATCTTACA
ACCAGAACTCAATTACCCCCTGCATACACTAATTCTTTCACACGTGGTGTTTATTA
CCCTGACAAAGTTTTCAGATCCTCAGTTTTACATTCAACTCAGGACTTGTTCTTAC
CTTTCTTTTCCAATGTTACTTGGTTCCATGCTATACATGTCTCTGGGACCAATGGT
ACTAAGAGGTTTGATAACCCTGTCCTACCATTTAATGATGGTGTTTATTTTGCTTC
CACTGAGAAGTCTAACATAATAAGAGGCTGGATTTTTGGTACTACTTTAGATTCG
AAGACCCAGTCCCTACTTATTGTTAATAACGCTACTAATGTTGTTATTAAAGTCTG
TGAATTTCAATTTTGTAATGATCCATTTTTGGGTGTTTATTACCACAAAAACAACA
AAAGTTGGATGGAAAGTGAGTTCAGAGTTTATTCTAGTGCGAATAATTGCACTTT
TGAATATGTCTCTCAGCCTTTTCTTATGGACCTTGAAGGAAAACAGGGTAATTTC
AAAAATCTTAGGGAATTTGTGTTTAAGAATATTGATGGTTATTTTAAAATATATT
CTAAGCACACGCCTATTAATTTAGTGCGTGATCTCCCTCAGGGTTTTTCGGCTTTA
GAACCATTGGTAGATTTGCCAATAGGTATTAACATCACTAGGTTTCAAACTTTAC
TTGCTTTACATAGAAGTTATTTGACTCCTGGTGATTCTTCTTCAGGTTGGACAGCT
GGTGCTGCAGCTTATTATGTGGGTTATCTTCAACCTAGGACTTTTCTATTAAAATA
TAATGAAAATGGAACCATTACAGATGCTGTAGACTGTGCACTTGACCCTCTCTCA
GAAACAAAGTGTACGTTGAAATCCTTCACTGTAGAAAAAGGAATCTATCAAACT
TCTAACTTTAGAGTCCAACCAACAGAATCTATTGTTAGATTTCCTAATATTACAA
ACTTGTGCCCTTTTGGTGAAGTTTTTAACGCCACCAGATTTGCATCTGTTTATGCT
TGGAACAGGAAGAGAATCAGCAACTGTGTTGCTGATTATTCTGTCCTATATAATT
CCGCATCATTTTCCACTTTTAAGTGTTATGGAGTGTCTCCTACTAAATTAAATGAT
CTCTGCTTTACTAATGTCTATGCAGATTCATTTGTAATTAGAGGTGATGAAGTCA
GACAAATCGCTCCAGGGCAAACTGGAAAGATTGCTGATTATAATTATAAATTAC
CAGATGATTTTACAGGCTGCGTTATAGCTTGGAATTCTAACAATCTTGATTCTAA
GGTTGGTGGTAATTATAATTACCTGTATAGATTGTTTAGGAAGTCTAATCTCAAA
CCTTTTGAGAGAGATATTTCAACTGAAATCTATCAGGCCGGTAGCACACCTTGTA
ATGGTGTTGAAGGTTTTAATTGTTACTTTCCTTTACAATCATATGGTTTCCAACCC
ACTAATGGTGTTGGTTACCAACCATACAGAGTAGTAGTACTTTCTTTTGAACTTCT
ACATGCACCAGCAACTGTTTGTGGACCTAAAAAGTCTACTAATTTGGTTAAAAAC
AAATGTGTCAATTTCAACTTCAATGGTTTAACAGGCACAGGTGTTCTTACTGAGT
CTAACAAAAAGTTTCTGCCTTTCCAACAATTTGGCAGAGACATTGCTGACACTAC
TGATGCTGTCCGTGATCCACAGACACTTGAGATTCTTGACATTACACCATGTTCTT
TTGGTGGTGTCAGTGTTATAACACCAGGAACAAATACTTCTAACCAGGTTGCTGT
TCTTTATCAGGATGTTAACTGCACAGAAGTCCCTGTTGCTATTCATGCAGATCAA
CTTACTCCTACTTGGCGTGTTTATTCTACAGGTTCTAATGTTTTTCAAACACGTGC
AGGCTGTTTAATAGGGGCTGAACATGTCAACAACTCATATGAGTGTGACATACCC
ATTGGTGCAGGTATATGCGCTAGTTATCAGACTCAGACTAATTCTCCTCGGCGGG
CACGTAGTGTAGCTAGTCAATCCATCATTGCCTACACTATGTCACTTGGTGCAGA
AAATTCAGTTGCTTACTCTAATAACTCTATTGCCATACCCACAAATTTTACTATTA
GTGTTACCACAGAAATTCTACCAGTGTCTATGACCAAGACATCAGTAGATTGTAC
AATGTACATTTGTGGTGATTCAACTGAATGCAGCAATCTTTTGTTGCAATATGGC
AGTTTTTGTACACAATTAAACCGTGCTTTAACTGGAATAGCTGTTGAACAAGACA
AAAACACCCAAGAAGTTTTTGCACAAGTCAAACAAATTTACAAAACACCACCAA
TTAAAGATTTTGGTGGTTTTAATTTTTCACAAATATTACCAGATCCATCAAAACCA
AGCAAGAGGTCATTTATTGAAGATCTACTTTTCAACAAAGTGACACTTGCAGATG
CTGGCTTCATCAAACAATATGGTGATTGCCTTGGTGATATTGCTGCTAGAGACCT
CATTTGTGCACAAAAGTTTAACGGCCTTACTGTTTTGCCACCTTTGCTCACAGATG
AAATGATTGCTCAATACACTTCTGCACTGTTAGCGGGTACAATCACTTCTGGTTG
GACCTTTGGTGCAGGTGCTGCATTACAAATACCATTTGCTATGCAAATGGCTTAT
AGGTTTAATGGTATTGGAGTTACACAGAATGTTCTCTATGAGAACCAAAAATTGA
TTGCCAACCAATTTAATAGTGCTATTGGCAAAATTCAAGACTCACTTTCTTCCAC
AGCAAGTGCACTTGGAAAACTTCAAGATGTGGTCAACCAAAATGCACAAGCTTT
AAACACGCTTGTTAAACAACTTAGCTCCAATTTTGGTGCAATTTCAAGTGTTTTA
AATGATATCCTTTCACGTCTTGACAAAGTTGAGGCTGAAGTGCAAATTGATAGGT
TGATCACAGGCAGACTTCAAAGTTTGCAGACATATGTGACTCAACAATTAATTAG
AGCTGCAGAAATCAGAGCTTCTGCTAATCTTGCTGCTACTAAAATGTCAGAGTGT
GTACTTGGACAATCAAAAAGAGTTGATTTTTGTGGAAAGGGCTATCATCTTATGT
CCTTCCCTCAGTCAGCACCTCATGGTGTAGTCTTCTTGCATGTGACTTATGTCCCT
GCACAAGAAAAGAACTTCACAACTGCTCCTGCCATTTGTCATGATGGAAAAGCA
CACTTTCCTCGTGAAGGTGTCTTTGTTTCAAATGGCACACACTGGTTTGTAACAC
AAAGGAATTTTTATGAACCACAAATCATTACTACAGACAACACATTTGTGTCTGG
TAACTGTGATGTTGTAATAGGAATTGTCAACAACACAGTTTATGATCCTTTGCAA
CCTGAATTAGACTCATTCAAGGAGGAGTTAGATAAATATTTTAAGAATCATACAT
CACCAGATGTTGATTTAGGTGACATCTCTGGCATTAATGCTTCAGTTGTAAACAT
TCAAAAAGAAATTGACCGCCTCAATGAGGTTGCCAAGAATTTAAATGAATCTCTC
ATCGATCTCCAAGAACTTGGAAAGTATGAGCAGTATATAAAATGGCCATGGTAC
ATTTGGCTAGGTTTTATAGCTGGCTTGATTGCCATAGTAATGGTGACAATTATGCT
TTGCTGTATGACCAGTTGCTGTAGTTGTCTCAAGGGCTGTTGTTCTTGTGGATCCT
GCTGCAAATTTGATGAAGACGACTCTGAGCCAGTGCTCAAAGGAGTCAAATTAC
ATTACACATAA
SEQ ID NO: 122
ATGTTCGTCTTCCTGGTCCTGCTGCCTCTGGTCTCCTCACAGTGCGTCAATCTGAC
AACTCGGACTCAGCTGCCACCTGCTTATACTAATAGCTTCACCAGAGGCGTGTAC
TATCCTGACAAGGTGTTTAGAAGCTCCGTGCTGCACTCTACACAGGATCTGTTTC
TGCCATTCTTTAGCAACGTGACCTGGTTCCACGCCATCCACGTGAGCGGCACCAA
TGGCACAAAGCGGTTCGACAATCCCGTGCTGCCTTTTAACGATGGCGTGTACTTC
GCCTCTACCGAGAAGTCCAACATCATCAGAGGCTGGATCTTTGGCACCACACTGG
ACTCCAAGACACAGTCTCTGCTGATCGTGAACAATGCCACCAACGTGGTCATCAA
GGTGTGCGAGTTCCAGTTTTGTAATGATCCCTTCCTGGGCGTGTACTATCACAAG
AACAATAAGAGCTGGATGGAGTCCGAGTTTAGAGTGTATTCTAGCGCCAACAAC
TGCACATTTGAGTACGTGAGCCAGCCTTTCCTGATGGACCTGGAGGGCAAGCAG
GGCAATTTCAAGAACCTGAGGGAGTTCGTGTTTAAGAATATCGACGGCTACTTCA
AAATCTACTCTAAGCACACCCCCATCAACCTGGTGCGCGACCTGCCTCAGGGCTT
CAGCGCCCTGGAGCCCCTGGTGGATCTGCCTATCGGCATCAACATCACCCGGTTT
CAGACACTGCTGGCCCTGCACAGAAGCTACCTGACACCCGGCGACTCCTCTAGC
GGATGGACCGCCGGCGCTGCCGCCTACTATGTGGGCTACCTCCAGCCCCGGACCT
TCCTGCTGAAGTACAACGAGAATGGCACCATCACAGACGCAGTGGATTGCGCCC
TGGACCCCCTGAGCGAGACAAAGTGTACACTGAAGTCCTTTACCGTGGAGAAGG
GCATCTATCAGACATCCAATTTCAGGGTGCAGCCAACCGAGTCTATCGTGCGCTT
TCCTAATATCACAAACCTGTGCCCATTTGGCGAGGTGTTCAACGCAACCCGCTTC
GCCAGCGTGTACGCCTGGAATAGGAAGCGGATCAGCAACTGCGTGGCCGACTAT
AGCGTGCTGTACAACTCCGCCTCTTTCAGCACCTTTAAGTGCTATGGCGTGTCCC
CCACAAAGCTGAATGACCTGTGCTTTACCAACGTCTACGCCGATTCTTTCGTGAT
CAGGGGCGACGAGGTGCGCCAGATCGCCCCCGGCCAGACAGGCAAGATCGCAG
ACTACAATTATAAGCTGCCAGACGATTTCACCGGCTGCGTGATCGCCTGGAACAG
CAACAATCTGGATTCCAAAGTGGGCGGCAACTACAATTATCTGTACCGGCTGTTT
AGAAAGAGCAATCTGAAGCCCTTCGAGAGGGACATCTCTACAGAAATCTACCAG
GCCGGCAGCACCCCTTGCAATGGCGTGGAGGGCTTTAACTGTTATTTCCCACTCC
AGTCCTACGGCTTCCAGCCCACAAACGGCGTGGGCTATCAGCCTTACCGCGTGGT
GGTGCTGAGCTTTGAGCTGCTGCACGCCCCAGCAACAGTGTGCGGCCCCAAGAA
GTCCACCAATCTGGTGAAGAACAAGTGCGTGAACTTCAACTTCAACGGCCTGAC
CGGCACAGGCGTGCTGACCGAGTCCAACAAGAAGTTCCTGCCATTTCAGCAGTTC
GGCAGGGACATCGCAGATACCACAGACGCCGTGCGCGACCCACAGACCCTGGAG
ATCCTGGACATCACACCCTGCTCTTTCGGCGGCGTGAGCGTGATCACACCCGGCA
CCAATACAAGCAACCAGGTGGCCGTGCTGTATCAGGACGTGAATTGTACCGAGG
TGCCCGTGGCTATCCACGCCGATCAGCTGACCCCAACATGGCGGGTGTACAGCA
CCGGCTCCAACGTCTTCCAGACAAGAGCCGGATGCCTGATCGGAGCAGAGCACG
TGAACAATTCCTATGAGTGCGACATCCCAATCGGCGCCGGCATCTGTGCCTCTTA
CCAGACCCAGACAAACTCTCCCAGACGGGCCCGGAGCGTGGCCTCCCAGTCTAT
CATCGCCTATACCATGTCCCTGGGCGCCGAGAACAGCGTGGCCTACTCTAACAAT
AGCATCGCCATCCCAACCAACTTCACAATCTCTGTGACCACAGAGATCCTGCCCG
TGTCCATGACCAAGACATCTGTGGACTGCACAATGTATATCTGTGGCGATTCTAC
CGAGTGCAGCAACCTGCTGCTCCAGTACGGCAGCTTTTGTACCCAGCTGAATAGA
GCCCTGACAGGCATCGCCGTGGAGCAGGATAAGAACACACAGGAGGTGTTCGCC
CAGGTGAAGCAAATCTACAAGACCCCCCCTATCAAGGACTTTGGCGGCTTCAATT
TTTCCCAGATCCTGCCTGATCCATCCAAGCCTTCTAAGCGGAGCTTTATCGAGGA
CCTGCTGTTCAACAAGGTGACCCTGGCCGATGCCGGCTTCATCAAGCAGTATGGC
GATTGCCTGGGCGACATCGCAGCCAGGGACCTGATCTGCGCCCAGAAGTTTAAT
GGCCTGACCGTGCTGCCACCCCTGCTGACAGATGAGATGATCGCACAGTACACA
AGCGCCCTGCTGGCCGGCACCATCACATCCGGATGGACCTTCGGCGCAGGAGCC
GCCCTCCAGATCCCCTTTGCCATGCAGATGGCCTATAGGTTCAACGGCATCGGCG
TGACCCAGAATGTGCTGTACGAGAACCAGAAGCTGATCGCCAATCAGTTTAACT
CCGCCATCGGCAAGATCCAGGACAGCCTGTCCTCTACAGCCAGCGCCCTGGGCA
AGCTCCAGGATGTGGTGAATCAGAACGCCCAGGCCCTGAATACCCTGGTGAAGC
AGCTGAGCAGCAACTTCGGCGCCATCTCTAGCGTGCTGAATGACATCCTGAGCCG
GCTGGACAAGGTGGAGGCAGAGGTGCAGATCGACCGGCTGATCACCGGCCGGCT
CCAGAGCCTCCAGACCTATGTGACACAGCAGCTGATCAGGGCCGCCGAGATCAG
GGCCAGCGCCAATCTGGCAGCAACCAAGATGTCCGAGTGCGTGCTGGGCCAGTC
TAAGAGAGTGGACTTTTGTGGCAAGGGCTATCACCTGATGTCCTTCCCTCAGTCT
GCCCCACACGGCGTGGTGTTTCTGCACGTGACCTACGTGCCCGCCCAGGAGAAG
AACTTCACCACAGCCCCTGCCATCTGCCACGATGGCAAGGCCCACTTTCCAAGGG
AGGGCGTGTTCGTGTCCAACGGCACCCACTGGTTTGTGACACAGCGCAATTTCTA
CGAGCCCCAGATCATCACCACAGACAACACCTTCGTGAGCGGCAACTGTGACGT
GGTCATCGGCATCGTGAACAATACCGTGTATGATCCACTCCAGCCCGAGCTGGAC
AGCTTTAAGGAGGAGCTGGATAAGTATTTCAAGAATCACACCTCCCCTGACGTG
GATCTGGGCGACATCAGCGGCATCAATGCCTCCGTGGTGAACATCCAGAAGGAG
ATCGACCGCCTGAACGAGGTGGCTAAGAATCTGAACGAGAGCCTGATCGACCTC
CAGGAGCTGGGCAAGTATGAGCAGTACATCAAGTGGCCCTGGTACATCTGGCTG
GGCTTCATCGCCGGCCTGATCGCCATCGTGATGGTGACCATCATGCTGTGCTGTA
TGACATCCTGCTGTTCTTGCCTGAAGGGCTGCTGTAGCTGTGGCTCCTGCTGTAA
GTTTGACGAGGATGACTCTGAACCTGTGCTGAAGGGCGTGAAGCTGCATTACAC
CTAA
SEQ ID NO: 123
MFVFLVLLPLVSSQCVNLTTRTQLPPAYTNSFTRGVYYPDKVFRSSVLHSTQDLFLPF
FSNVTWFHAIHVSGTNGTKRFDNPVLPFNDGVYFASTEKSNIIRGWIFGTTLDSKTQS
LLIVNNATNVVIKVCEFQFCNDPFLGVYYHKNNKSWMESEFRVYSSANNCTFEYVS
QPFLMDLEGKQGNFKNLREFVFKNIDGYFKIYSKHTPINLVRDLPQGFSALEPLVDLP
IGINITRFQTLLALHRSYLTPGDSSSGWTAGAAAYYVGYLQPRTFLLKYNENGTITDA
VDCALDPLSETKCTLKSFTVEKGIYQTSNFRVQPTESIVRFPNITNLCPFGEVFNATRF
ASVYAWNRKRISNCVADYSVLYNSASFSTFKCYGVSPTKLNDLCFTNVYADSFVIRG
DEVRQIAPGQTGKIADYNYKLPDDFTGCVIAWNSNNLDSKVGGNYNYLYRLFRKSN
LKPFERDISTEIYQAGSTPCNGVEGENCYFPLQSYGFQPTNGVGYQPYRVVVLSFELL
HAPATVCGPKKSTNLVKNKCVNFNFNGLTGTGVLTESNKKFLPFQQFGRDIADTTD
AVRDPQTLEILDITPCSFGGVSVITPGTNTSNQVAVLYQDVNCTEVPVAIHADQLTPT
WRVYSTGSNVFQTRAGCLIGAEHVNNSYECDIPIGAGICASYQTQTNSPRRARSVAS
QSIIAYTMSLGAENSVAYSNNSIAIPTNFTISVTTEILPVSMTKTSVDCTMYICGDSTEC
SNLLLQYGSFCTQLNRALTGIAVEQDKNTQEVFAQVKQIYKTPPIKDFGGFNFSQILP
DPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLI
ANQFNSAIGKIQDSLSSTASALGKLQDVVNQNAQALNTLVKQLSSNFGAISSVLNDIL
SRLDKVEAEVQIDRLITGRLQSLQTYVTQQLIRAAEIRASANLAATKMSECVLGQSK
RVDFCGKGYHLMSFPQSAPHGVVFLHVTYVPAQEKNFTTAPAICHDGKAHFPREGV
FVSNGTHWFVTQRNFYEPQIITTDNTFVSGNCDVVIGIVNNTVYDPLQPELDSFKEEL
DKYFKNHTSPDVDLGDISGINASVVNIQKEIDRLNEVAKNLNESLIDLQELGKYEQYI
KWPWYIWLGFIAGLIAIVMVTIMLCCMTSCCSCLKGCCSCGSCCKFDEDDSEPVLKG
VKLHYT
SEQ ID NO: 77
CCTGAATGGACTACGACATAGTCTAGTCCGCCAAGGCCGCCACC
SEQ ID NO: 78
ATGGGCGGCGCATGAGAGAAGCCCAGACCAATTACCTACCCAAAATGGAGAAA
GTTCACGTTGACATCGAGGAAGACAGCCCATTCCTCAGAGCTTTGCAGCGGAGCT
TCCCGCAGTTTGAGGTAGAAGCCAAGCAGGTCACTGATAATGACCATGCTAATG
CCAGAGCGTTTTCGCATCTGGCTTCAAAACTGATCGAAACGGAGGTGGACCCATC
CGACACGATCCTTGACATTGGAAGTGCGCCCGCCCGCAGAATGTATTCTAAGCAC
AAGTATCATTGTATCTGTCCGATGAGATGTGCGGAAGATCCGGACAGATTGTATA
AGTATGCAACTAAGCTGAAGAAAAACTGTAAGGAAATAACTGATAAGGAATTGG
ACAAGAAAATGAAGGAGCTGGCCGCCGTCATGAGCGACCCTGACCTGGAAACTG
AGACTATGTGCCTCCACGACGACGAGTCGTGTCGCTACGAAGGGCAAGTCGCTG
TTTACCAGGATGTATACGCCGTCGACGGCCCCACCAGCCTGTACCACCAGGCCAA
CAAGGGCGTGAGGGTGGCCTACTGGATCGGCTTCGACACCACACCCTTCATGTTC
AAGAACCTGGCCGGCGCCTACCCCAGCTACAGCACCAACTGGGCCGACGAGACC
GTGCTGACCGCCAGGAACATCGGCCTGTGCAGCAGCGACGTGATGGAGAGGAGC
CGGAGAGGCATGAGCATCCTGAGGAAGAAATACCTGAAGCCCAGCAACAACGT
GCTGTTCAGCGTGGGCAGCACCATCTACCACGAGAAGAGGGACCTGCTCAGGAG
CTGGCACCTGCCCAGCGTGTTCCACCTGAGGGGCAAGCAGAACTACACCTGCAG
GTGCGAGACCATCGTGAGCTGCGACGGCTACGTGGTGAAGAGGATCGCCATCAG
CCCCGGCCTGTACGGCAAGCCCAGCGGCTACGCCGCTACAATGCACAGGGAGGG
CTTCCTGTGCTGCAAGGTGACCGACACCCTGAACGGCGAGAGGGTGAGCTTCCC
CGTGTGCACCTACGTGCCCGCCACCCTGTGCGACCAGATGACCGGCATCCTGGCC
ACCGACGTGAGCGCCGACGACGCCCAGAAGCTGCTCGTGGGCCTGAACCAGAGG
ATCGTGGTCAACGGCAGGACCCAGAGGAACACCAACACAATGAAGAACTACCTG
CTGCCCGTGGTGGCCCAGGCTTTCGCCAGGTGGGCCAAGGAGTACAAGGAGGAC
CAGGAAGACGAGAGGCCCCTGGGCCTGAGGGACAGGCAGCTGGTGATGGGCTG
CTGCTGGGCCTTCAGGCGGCACAAGATCACCAGCATCTACAAGAGGCCCGACAC
CCAGACCATCATCAAGGTGAACAGCGACTTCCACAGCTTCGTGCTGCCCAGGATC
GGCAGCAACACCCTGGAGATCGGCCTGAGGACCCGGATCAGGAAGATGCTGGAG
GAACACAAGGAGCCCAGCCCACTGATCACCGCCGAGGACGTGCAGGAGGCCAA
GTGCGCTGCCGACGAGGCCAAGGAGGTGAGGGAGGCCGAGGAACTGAGGGCCG
CCCTGCCACCCCTGGCTGCCGACGTGGAGGAACCCACCCTGGAAGCCGACGTGG
ACCTGATGCTGCAGGAGGCCGGCGCCGGAAGCGTGGAGACACCCAGGGGCCTGA
TCAAGGTGACCAGCTACGACGGCGAGGACAAGATCGGCAGCTACGCCGTGCTGA
GCCCACAGGCCGTGCTGAAGTCCGAGAAGCTGAGCTGCATCCACCCACTGGCCG
AGCAGGTGATCGTGATCACCCACAGCGGCAGGAAGGGCAGGTACGCCGTGGAGC
CCTACCACGGCAAGGTGGTCGTGCCCGAGGGCCACGCCATCCCCGTGCAGGACT
TCCAGGCCCTGAGCGAGAGCGCCACCATCGTGTACAACGAGAGGGAGTTCGTGA
ACAGGTACCTGCACCATATCGCCACCCACGGCGGAGCCCTGAACACCGACGAGG
AATACTACAAGACCGTGAAGCCCAGCGAGCACGACGGCGAGTACCTGTACGACA
TCGACAGGAAGCAGTGCGTGAAGAAAGAGCTGGTGACCGGCCTGGGACTGACCG
GCGAGCTGGTGGACCCACCCTTCCACGAGTTCGCCTACGAGAGCCTGAGGACCA
GACCCGCCGCTCCCTACCAGGTGCCCACCATCGGCGTGTACGGCGTGCCCGGCA
GCGGAAAGAGCGGCATCATCAAGAGCGCCGTGACCAAGAAAGACCTGGTGGTC
AGCGCCAAGAAAGAGAACTGCGCCGAGATCATCAGGGACGTGAAGAAGATGAA
AGGCCTGGACGTGAACGCGCGCACCGTGGACAGCGTGCTGCTGAACGGCTGCAA
GCACCCCGTGGAGACCCTGTACATCGACGAGGCCTTCGCTTGCCACGCCGGCACC
CTGAGGGCCCTGATCGCCATCATCAGGCCCAAGAAAGCCGTGCTGTGCGGCGAC
CCCAAGCAGTGCGGCTTCTTCAACATGATGTGCCTGAAGGTGCACTTCAACCACG
AGATCTGCACCCAGGTGTTCCACAAGAGCATCAGCAGGCGGTGCACCAAGAGCG
TGACCAGCGTCGTGAGCACCCTGTTCTACGACAAGAAAATGAGGACCACCAACC
CCAAGGAGACCAAAATCGTGATCGACACCACAGGCAGCACCAAGCCCAAGCAG
GACGACCTGATCCTGACCTGCTTCAGGGGCTGGGTGAAGCAGCTGCAGATCGAC
TACAAGGGCAACGAGATCATGACCGCCGCTGCCAGCCAGGGCCTGACCAGGAAG
GGCGTGTACGCCGTGAGGTACAAGGTGAACGAGAACCCACTGTACGCTCCCACC
AGCGAGCACGTGAACGTGCTGCTGACCAGGACCGAGGACAGGATCGTGTGGAAG
ACCCTGGCCGGCGACCCCTGGATCAAGACCCTGACCGCCAAGTACCCCGGCAAC
TTCACCGCCACCATCGAAGAGTGGCAGGCCGAGCACGACGCCATCATGAGGCAC
ATCCTGGAGAGGCCCGACCCCACCGACGTGTTCCAGAACAAGGCCAACGTGTGC
TGGGCCAAGGCCCTGGTGCCCGTGCTGAAGACCGCCGGCATCGACATGACCACA
GAGCAGTGGAACACCGTGGACTACTTCGAGACCGACAAGGCCCACAGCGCCGAG
ATCGTGCTGAACCAGCTGTGCGTGAGGTTCTTCGGCCTGGACCTGGACAGCGGCC
TGTTCAGCGCCCCCACCGTGCCACTGAGCATCAGGAACAACCACTGGGACAACA
GCCCCAGCCCAAACATGTACGGCCTGAACAAGGAGGTGGTCAGGCAGCTGAGCA
GGCGGTACCCACAGCTGCCCAGGGCCGTGGCCACCGGCAGGGTGTACGACATGA
ACACCGGCACCCTGAGGAACTACGACCCCAGGATCAACCTGGTGCCCGTGAACA
GGCGGCTGCCCCACGCCCTGGTGCTGCACCACAACGAGCACCCACAGAGCGACT
TCAGCTCCTTCGTGAGCAAGCTGAAAGGCAGGACCGTGCTGGTCGTGGGCGAGA
AGCTGAGCGTGCCCGGCAAGATGGTGGACTGGCTGAGCGACAGGCCCGAGGCCA
CCTTCCGGGCCAGGCTGGACCTCGGCATCCCCGGCGACGTGCCCAAGTACGACA
TCATCTTCGTGAACGTCAGGACCCCATACAAGTACCACCATTACCAGCAGTGCGA
GGACCACGCCATCAAGCTGAGCATGCTGACCAAGAAGGCCTGCCTGCACCTGAA
CCCCGGAGGCACCTGCGTGAGCATCGGCTACGGCTACGCCGACAGGGCCAGCGA
GAGCATCATTGGCGCCATCGCCAGGCTGTTCAAGTTCAGCAGGGTGTGCAAACC
CAAGAGCAGCCTGGAGGAAACCGAGGTGCTGTTCGTGTTCATCGGCTACGACCG
GAAGGCCAGGACCCACAACCCCTACAAGCTGAGCAGCACCCTGACAAACATCTA
CACCGGCAGCAGGCTGCACGAGGCCGGCTGCGCCCCCAGCTACCACGTGGTCAG
GGGCGATATCGCCACCGCCACCGAGGGCGTGATCATCAACGCTGCCAACAGCAA
GGGCCAGCCCGGAGGCGGAGTGTGCGGCGCCCTGTACAAGAAGTTCCCCGAGAG
CTTCGACCTGCAGCCCATCGAGGTGGGCAAGGCCAGGCTGGTGAAGGGCGCCGC
TAAGCACATCATCCACGCCGTGGGCCCCAACTTCAACAAGGTGAGCGAGGTGGA
AGGCGACAAGCAGCTGGCCGAAGCCTACGAGAGCATCGCCAAGATCGTGAACG
ACAATAACTACAAGAGCGTGGCCATCCCACTGCTCAGCACCGGCATCTTCAGCG
GCAACAAGGACAGGCTGACCCAGAGCCTGAACCACCTGCTCACCGCCCTGGACA
CCACCGATGCCGACGTGGCCATCTACTGCAGGGACAAGAAGTGGGAGATGACCC
TGAAGGAGGCCGTGGCCAGGCGGGAGGCCGTGGAAGAGATCTGCATCAGCGAC
GACTCCAGCGTGACCGAGCCCGACGCCGAGCTGGTGAGGGTGCACCCCAAGAGC
TCCCTGGCCGGCAGGAAGGGCTACAGCACCAGCGACGGCAAGACCTTCAGCTAC
CTGGAGGGCACCAAGTTCCACCAGGCCGCTAAGGACATCGCCGAGATCAACGCT
ATGTGGCCCGTGGCCACCGAGGCCAACGAGCAGGTGTGCATGTACATCCTGGGC
GAGAGCATGTCCAGCATCAGGAGCAAGTGCCCCGTGGAGGAAAGCGAGGCCAG
CACACCACCCAGCACCCTGCCCTGCCTGTGCATCCACGCTATGACACCCGAGAGG
GTGCAGCGGCTGAAGGCCAGCAGGCCCGAGCAGATCACCGTGTGCAGCTCCTTC
CCACTGCCCAAGTACAGGATCACCGGCGTGCAGAAGATCCAGTGCAGCCAGCCC
ATCCTGTTCAGCCCAAAGGTGCCCGCCTACATCCACCCCAGGAAGTACCTGGTGG
AGACCCCACCCGTGGACGAGACACCCGAGCCAAGCGCCGAGAACCAGAGCACC
GAGGGCACACCCGAGCAGCCACCCCTGATCACCGAGGACGAGACAAGGACCCG
GACCCCAGAGCCCATCATTATCGAGGAAGAGGAAGAGGACAGCATCAGCCTGCT
GAGCGACGGCCCCACCCACCAGGTGCTGCAGGTGGAGGCCGACATCCACGGCCC
ACCCAGCGTGTCCAGCTCCAGCTGGAGCATCCCACACGCCAGCGACTTCGACGT
GGACAGCCTGAGCATCCTGGACACCCTGGAGGGCGCCAGCGTGACCTCCGGCGC
CACCAGCGCCGAGACCAACAGCTACTTCGCCAAGAGCATGGAGTTCCTGGCCAG
GCCCGTGCCAGCTCCCAGGACCGTGTTCAGGAACCCACCCCACCCAGCTCCCAG
GACCAGGACCCCAAGCCTGGCTCCCAGCAGGGCCTGCAGCAGGACCAGCCTGGT
GAGCACCCCACCCGGCGTGAACAGGGTGATCACCAGGGAGGAACTGGAGGCCCT
GACACCCAGCAGGACCCCCAGCAGGTCCGTGAGCAGGACTAGTCTGGTGTCCAA
CCCACCCGGCGTGAACAGGGTGATCACCAGGGAGGAATTCGAGGCCTTCGTGGC
CCAGCAACAGAGACGGTTCGACGCCGGCGCCTACATCTTCAGCAGCGACACCGG
CCAGGGACACCTGCAGCAAAAGAGCGTGAGGCAGACCGTGCTGAGCGAGGTGG
TGCTGGAGAGGACCGAGCTGGAAATCAGCTACGCCCCCAGGCTGGACCAGGAGA
AGGAGGAACTGCTCAGGAAGAAACTGCAGCTGAACCCCACCCCAGCCAACAGG
AGCAGGTACCAGAGCAGGAAGGTGGAGAACATGAAGGCCATCACCGCCAGGCG
GATCCTGCAGGGCCTGGGACACTACCTGAAGGCCGAGGGCAAGGTGGAGTGCTA
CAGGACCCTGCACCCCGTGCCACTGTACAGCTCCAGCGTGAACAGGGCCTTCTCC
AGCCCCAAGGTGGCCGTGGAGGCCTGCAACGCTATGCTGAAGGAGAACTTCCCC
ACCGTGGCCAGCTACTGCATCATCCCCGAGTACGACGCCTACCTGGACATGGTGG
ACGGCGCCAGCTGCTGCCTGGACACCGCCAGCTTCTGCCCCGCCAAGCTGAGGA
GCTTCCCCAAGAAACACAGCTACCTGGAGCCCACCATCAGGAGCGCCGTGCCCA
GCGCCATCCAGAACACCCTGCAGAACGTGCTGGCCGCTGCCACCAAGAGGAACT
GCAACGTGACCCAGATGAGGGAGCTGCCCGTGCTGGACAGCGCTGCCTTCAACG
TGGAGTGCTTCAAGAAATACGCCTGCAACAACGAGTACTGGGAGACCTTCAAGG
AGAACCCCATCAGGCTGACCGAAGAGAACGTGGTGAACTACATCACCAAGCTGA
AGGGCCCCAAGGCCGCTGCCCTGTTCGCTAAGACCCACAACCTGAACATGCTGC
AGGACATCCCAATGGACAGGTTCGTGATGGACCTGAAGAGGGACGTGAAGGTGA
CACCCGGCACCAAGCACACCGAGGAGAGGCCCAAGGTGCAGGTGATCCAGGCC
GCTGACCCACTGGCCACCGCCTACCTGTGCGGCATCCACAGGGAGCTGGTGAGG
CGGCTGAACGCCGTGCTGCTGCCCAACATCCACACCCTGTTCGACATGAGCGCCG
AGGACTTCGACGCCATCATCGCCGAGCACTTCCAGCCCGGCGACTGCGTGCTGG
AGACCGACATCGCCAGCTTCGACAAGAGCGAGGATGACGCTATGGCCCTGACCG
CTCTGATGATCCTGGAGGACCTGGGCGTGGACGCCGAGCTGCTCACCCTGATCGA
GGCTGCCTTCGGCGAGATCAGCTCCATCCACCTGCCCACCAAGACCAAGTTCAAG
TTCGGCGCTATGATGAAAAGCGGAATGTTCCTGACCCTGTTCGTGAACACCGTGA
TCAACATTGTGATCGCCAGCAGGGTGCTGCGGGAGAGGCTGACCGGCAGCCCCT
GCGCTGCCTTCATCGGCGACGACAACATCGTGAAGGGCGTGAAAAGCGACAAGC
TGATGGCCGACAGGTGCGCCACCTGGCTGAACATGGAGGTGAAGATCATCGACG
CCGTGGTGGGCGAGAAGGCCCCCTACTTCTGCGGCGGATTCATCCTGTGCGACAG
CGTGACCGGCACCGCCTGCAGGGTGGCCGACCCCCTGAAGAGGCTGTTCAAGCT
GGGCAAGCCACTGGCCGCTGACGATGAGCACGACGATGACAGGCGGAGGGCCCT
GCACGAGGAAAGCACCAGGTGGAACAGGGTGGGCATCCTGAGCGAGCTGTGCA
AGGCCGTGGAGAGCAGGTACGAGACCGTGGGCACCAGCATCATCGTGATGGCTA
TGACCACACTGGCCAGCTCCGTCAAGAGCTTCTCCTACCTGAGGGGGGCCCCTAT
AACTCTCTACGGCTAACCTGAATGGACTACGACATAGTCTAGTCCGCCAAGGCCG
CCACCACTCGAGTATGTTACGTGCAAAGGTGATTGTCACCCCCCGAAAGACCATA
TTGTGACACACCCTCAGTATCACGCCCAAACATTTACAGCCGCGGTGTCAAAAAC
CGCGTGGACGTGGTTAACATCCCTGCTGGGAGGATCAGCCGTAATTATTATAATT
GGCTTGGTGCTGGCTACTATTGTGGCCATGTACGTGCTGACCAACCAGAAACATA
ATTGAATACAGCAGCAATTGGCAAGCTGCTTACATAGAACTCGCGGCGATTGGC
ATGCCGCCTTAAAATTTTTATTTTATTTTTTCTTTTCTTTTCCGAATCGGATTTTGT
TTTTAATATTTCAAAAAAAAAAAAAAAAAAAAAAAAATCTAGAAAAAAAAAAA
AAAAAAAAAAAAAAAAAAAAAAAAAAAAAAAAAAAAAAAAAAAAAAAAAAAA
AAAAAAAAAAAAAAAAAAAAAAAAAAAAAAAAAAAAA
SEQ ID NO: 124
ATGGGCGGCGCATGAGAGAAGCCCAGACCAATTACCTACCCAAAATGGAGAAA
GTTCACGTTGACATCGAGGAAGACAGCCCATTCCTCAGAGCTTTGCAGCGGAGCT
TCCCGCAGTTTGAGGTAGAAGCCAAGCAGGTCACTGATAATGACCATGCTAATG
CCAGAGCGTTTTCGCATCTGGCTTCAAAACTGATCGAAACGGAGGTGGACCCATC
CGACACGATCCTTGACATTGGAAGTGCGCCCGCCCGCAGAATGTATTCTAAGCAC
AAGTATCATTGTATCTGTCCGATGAGATGTGCGGAAGATCCGGACAGATTGTATA
AGTATGCAACTAAGCTGAAGAAAAACTGTAAGGAAATAACTGATAAGGAATTGG
ACAAGAAAATGAAGGAGCTGGCCGCCGTCATGAGCGACCCTGACCTGGAAACTG
AGACTATGTGCCTCCACGACGACGAGTCGTGTCGCTACGAAGGGCAAGTCGCTG
TTTACCAGGATGTATACGCCGTCGACGGCCCCACCAGCCTGTACCACCAGGCCAA
CAAGGGCGTGAGGGTGGCCTACTGGATCGGCTTCGACACCACACCCTTCATGTTC
AAGAACCTGGCCGGCGCCTACCCCAGCTACAGCACCAACTGGGCCGACGAGACC
GTGCTGACCGCCAGGAACATCGGCCTGTGCAGCAGCGACGTGATGGAGAGGAGC
CGGAGAGGCATGAGCATCCTGAGGAAGAAATACCTGAAGCCCAGCAACAACGT
GCTGTTCAGCGTGGGCAGCACCATCTACCACGAGAAGAGGGACCTGCTCAGGAG
CTGGCACCTGCCCAGCGTGTTCCACCTGAGGGGCAAGCAGAACTACACCTGCAG
GTGCGAGACCATCGTGAGCTGCGACGGCTACGTGGTGAAGAGGATCGCCATCAG
CCCCGGCCTGTACGGCAAGCCCAGCGGCTACGCCGCTACAATGCACAGGGAGGG
CTTCCTGTGCTGCAAGGTGACCGACACCCTGAACGGCGAGAGGGTGAGCTTCCC
CGTGTGCACCTACGTGCCCGCCACCCTGTGCGACCAGATGACCGGCATCCTGGCC
ACCGACGTGAGCGCCGACGACGCCCAGAAGCTGCTCGTGGGCCTGAACCAGAGG
ATCGTGGTCAACGGCAGGACCCAGAGGAACACCAACACAATGAAGAACTACCTG
CTGCCCGTGGTGGCCCAGGCTTTCGCCAGGTGGGCCAAGGAGTACAAGGAGGAC
CAGGAAGACGAGAGGCCCCTGGGCCTGAGGGACAGGCAGCTGGTGATGGGCTG
CTGCTGGGCCTTCAGGCGGCACAAGATCACCAGCATCTACAAGAGGCCCGACAC
CCAGACCATCATCAAGGTGAACAGCGACTTCCACAGCTTCGTGCTGCCCAGGATC
GGCAGCAACACCCTGGAGATCGGCCTGAGGACCCGGATCAGGAAGATGCTGGAG
GAACACAAGGAGCCCAGCCCACTGATCACCGCCGAGGACGTGCAGGAGGCCAA
GTGCGCTGCCGACGAGGCCAAGGAGGTGAGGGAGGCCGAGGAACTGAGGGCCG
CCCTGCCACCCCTGGCTGCCGACGTGGAGGAACCCACCCTGGAAGCCGACGTGG
ACCTGATGCTGCAGGAGGCCGGCGCCGGAAGCGTGGAGACACCCAGGGGCCTGA
TCAAGGTGACCAGCTACGACGGCGAGGACAAGATCGGCAGCTACGCCGTGCTGA
GCCCACAGGCCGTGCTGAAGTCCGAGAAGCTGAGCTGCATCCACCCACTGGCCG
AGCAGGTGATCGTGATCACCCACAGCGGCAGGAAGGGCAGGTACGCCGTGGAGC
CCTACCACGGCAAGGTGGTCGTGCCCGAGGGCCACGCCATCCCCGTGCAGGACT
TCCAGGCCCTGAGCGAGAGCGCCACCATCGTGTACAACGAGAGGGAGTTCGTGA
ACAGGTACCTGCACCATATCGCCACCCACGGCGGAGCCCTGAACACCGACGAGG
AATACTACAAGACCGTGAAGCCCAGCGAGCACGACGGCGAGTACCTGTACGACA
TCGACAGGAAGCAGTGCGTGAAGAAAGAGCTGGTGACCGGCCTGGGACTGACCG
GCGAGCTGGTGGACCCACCCTTCCACGAGTTCGCCTACGAGAGCCTGAGGACCA
GACCCGCCGCTCCCTACCAGGTGCCCACCATCGGCGTGTACGGCGTGCCCGGCA
GCGGAAAGAGCGGCATCATCAAGAGCGCCGTGACCAAGAAAGACCTGGTGGTC
AGCGCCAAGAAAGAGAACTGCGCCGAGATCATCAGGGACGTGAAGAAGATGAA
AGGCCTGGACGTGAACGCGCGCACCGTGGACAGCGTGCTGCTGAACGGCTGCAA
GCACCCCGTGGAGACCCTGTACATCGACGAGGCCTTCGCTTGCCACGCCGGCACC
CTGAGGGCCCTGATCGCCATCATCAGGCCCAAGAAAGCCGTGCTGTGCGGCGAC
CCCAAGCAGTGCGGCTTCTTCAACATGATGTGCCTGAAGGTGCACTTCAACCACG
AGATCTGCACCCAGGTGTTCCACAAGAGCATCAGCAGGCGGTGCACCAAGAGCG
TGACCAGCGTCGTGAGCACCCTGTTCTACGACAAGAAAATGAGGACCACCAACC
CCAAGGAGACCAAAATCGTGATCGACACCACAGGCAGCACCAAGCCCAAGCAG
GACGACCTGATCCTGACCTGCTTCAGGGGCTGGGTGAAGCAGCTGCAGATCGAC
TACAAGGGCAACGAGATCATGACCGCCGCTGCCAGCCAGGGCCTGACCAGGAAG
GGCGTGTACGCCGTGAGGTACAAGGTGAACGAGAACCCACTGTACGCTCCCACC
AGCGAGCACGTGAACGTGCTGCTGACCAGGACCGAGGACAGGATCGTGTGGAAG
ACCCTGGCCGGCGACCCCTGGATCAAGACCCTGACCGCCAAGTACCCCGGCAAC
TTCACCGCCACCATCGAAGAGTGGCAGGCCGAGCACGACGCCATCATGAGGCAC
ATCCTGGAGAGGCCCGACCCCACCGACGTGTTCCAGAACAAGGCCAACGTGTGC
TGGGCCAAGGCCCTGGTGCCCGTGCTGAAGACCGCCGGCATCGACATGACCACA
GAGCAGTGGAACACCGTGGACTACTTCGAGACCGACAAGGCCCACAGCGCCGAG
ATCGTGCTGAACCAGCTGTGCGTGAGGTTCTTCGGCCTGGACCTGGACAGCGGCC
TGTTCAGCGCCCCCACCGTGCCACTGAGCATCAGGAACAACCACTGGGACAACA
GCCCCAGCCCAAACATGTACGGCCTGAACAAGGAGGTGGTCAGGCAGCTGAGCA
GGCGGTACCCACAGCTGCCCAGGGCCGTGGCCACCGGCAGGGTGTACGACATGA
ACACCGGCACCCTGAGGAACTACGACCCCAGGATCAACCTGGTGCCCGTGAACA
GGCGGCTGCCCCACGCCCTGGTGCTGCACCACAACGAGCACCCACAGAGCGACT
TCAGCTCCTTCGTGAGCAAGCTGAAAGGCAGGACCGTGCTGGTCGTGGGCGAGA
AGCTGAGCGTGCCCGGCAAGATGGTGGACTGGCTGAGCGACAGGCCCGAGGCCA
CCTTCCGGGCCAGGCTGGACCTCGGCATCCCCGGCGACGTGCCCAAGTACGACA
TCATCTTCGTGAACGTCAGGACCCCATACAAGTACCACCATTACCAGCAGTGCGA
GGACCACGCCATCAAGCTGAGCATGCTGACCAAGAAGGCCTGCCTGCACCTGAA
CCCCGGAGGCACCTGCGTGAGCATCGGCTACGGCTACGCCGACAGGGCCAGCGA
GAGCATCATTGGCGCCATCGCCAGGCTGTTCAAGTTCAGCAGGGTGTGCAAACC
CAAGAGCAGCCTGGAGGAAACCGAGGTGCTGTTCGTGTTCATCGGCTACGACCG
GAAGGCCAGGACCCACAACCCCTACAAGCTGAGCAGCACCCTGACAAACATCTA
CACCGGCAGCAGGCTGCACGAGGCCGGCTGCGCCCCCAGCTACCACGTGGTCAG
GGGCGATATCGCCACCGCCACCGAGGGCGTGATCATCAACGCTGCCAACAGCAA
GGGCCAGCCCGGAGGCGGAGTGTGCGGCGCCCTGTACAAGAAGTTCCCCGAGAG
CTTCGACCTGCAGCCCATCGAGGTGGGCAAGGCCAGGCTGGTGAAGGGCGCCGC
TAAGCACATCATCCACGCCGTGGGCCCCAACTTCAACAAGGTGAGCGAGGTGGA
AGGCGACAAGCAGCTGGCCGAAGCCTACGAGAGCATCGCCAAGATCGTGAACG
ACAATAACTACAAGAGCGTGGCCATCCCACTGCTCAGCACCGGCATCTTCAGCG
GCAACAAGGACAGGCTGACCCAGAGCCTGAACCACCTGCTCACCGCCCTGGACA
CCACCGATGCCGACGTGGCCATCTACTGCAGGGACAAGAAGTGGGAGATGACCC
TGAAGGAGGCCGTGGCCAGGCGGGAGGCCGTGGAAGAGATCTGCATCAGCGAC
GACTCCAGCGTGACCGAGCCCGACGCCGAGCTGGTGAGGGTGCACCCCAAGAGC
TCCCTGGCCGGCAGGAAGGGCTACAGCACCAGCGACGGCAAGACCTTCAGCTAC
CTGGAGGGCACCAAGTTCCACCAGGCCGCTAAGGACATCGCCGAGATCAACGCT
ATGTGGCCCGTGGCCACCGAGGCCAACGAGCAGGTGTGCATGTACATCCTGGGC
GAGAGCATGTCCAGCATCAGGAGCAAGTGCCCCGTGGAGGAAAGCGAGGCCAG
CACACCACCCAGCACCCTGCCCTGCCTGTGCATCCACGCTATGACACCCGAGAGG
GTGCAGCGGCTGAAGGCCAGCAGGCCCGAGCAGATCACCGTGTGCAGCTCCTTC
CCACTGCCCAAGTACAGGATCACCGGCGTGCAGAAGATCCAGTGCAGCCAGCCC
ATCCTGTTCAGCCCAAAGGTGCCCGCCTACATCCACCCCAGGAAGTACCTGGTGG
AGACCCCACCCGTGGACGAGACACCCGAGCCAAGCGCCGAGAACCAGAGCACC
GAGGGCACACCCGAGCAGCCACCCCTGATCACCGAGGACGAGACAAGGACCCG
GACCCCAGAGCCCATCATTATCGAGGAAGAGGAAGAGGACAGCATCAGCCTGCT
GAGCGACGGCCCCACCCACCAGGTGCTGCAGGTGGAGGCCGACATCCACGGCCC
ACCCAGCGTGTCCAGCTCCAGCTGGAGCATCCCACACGCCAGCGACTTCGACGT
GGACAGCCTGAGCATCCTGGACACCCTGGAGGGCGCCAGCGTGACCTCCGGCGC
CACCAGCGCCGAGACCAACAGCTACTTCGCCAAGAGCATGGAGTTCCTGGCCAG
GCCCGTGCCAGCTCCCAGGACCGTGTTCAGGAACCCACCCCACCCAGCTCCCAG
GACCAGGACCCCAAGCCTGGCTCCCAGCAGGGCCTGCAGCAGGACCAGCCTGGT
GAGCACCCCACCCGGCGTGAACAGGGTGATCACCAGGGAGGAACTGGAGGCCCT
GACACCCAGCAGGACCCCCAGCAGGTCCGTGAGCAGGACTAGTCTGGTGTCCAA
CCCACCCGGCGTGAACAGGGTGATCACCAGGGAGGAATTCGAGGCCTTCGTGGC
CCAGCAACAGAGACGGTTCGACGCCGGCGCCTACATCTTCAGCAGCGACACCGG
CCAGGGACACCTGCAGCAAAAGAGCGTGAGGCAGACCGTGCTGAGCGAGGTGG
TGCTGGAGAGGACCGAGCTGGAAATCAGCTACGCCCCCAGGCTGGACCAGGAGA
AGGAGGAACTGCTCAGGAAGAAACTGCAGCTGAACCCCACCCCAGCCAACAGG
AGCAGGTACCAGAGCAGGAAGGTGGAGAACATGAAGGCCATCACCGCCAGGCG
GATCCTGCAGGGCCTGGGACACTACCTGAAGGCCGAGGGCAAGGTGGAGTGCTA
CAGGACCCTGCACCCCGTGCCACTGTACAGCTCCAGCGTGAACAGGGCCTTCTCC
AGCCCCAAGGTGGCCGTGGAGGCCTGCAACGCTATGCTGAAGGAGAACTTCCCC
ACCGTGGCCAGCTACTGCATCATCCCCGAGTACGACGCCTACCTGGACATGGTGG
ACGGCGCCAGCTGCTGCCTGGACACCGCCAGCTTCTGCCCCGCCAAGCTGAGGA
GCTTCCCCAAGAAACACAGCTACCTGGAGCCCACCATCAGGAGCGCCGTGCCCA
GCGCCATCCAGAACACCCTGCAGAACGTGCTGGCCGCTGCCACCAAGAGGAACT
GCAACGTGACCCAGATGAGGGAGCTGCCCGTGCTGGACAGCGCTGCCTTCAACG
TGGAGTGCTTCAAGAAATACGCCTGCAACAACGAGTACTGGGAGACCTTCAAGG
AGAACCCCATCAGGCTGACCGAAGAGAACGTGGTGAACTACATCACCAAGCTGA
AGGGCCCCAAGGCCGCTGCCCTGTTCGCTAAGACCCACAACCTGAACATGCTGC
AGGACATCCCAATGGACAGGTTCGTGATGGACCTGAAGAGGGACGTGAAGGTGA
CACCCGGCACCAAGCACACCGAGGAGAGGCCCAAGGTGCAGGTGATCCAGGCC
GCTGACCCACTGGCCACCGCCTACCTGTGCGGCATCCACAGGGAGCTGGTGAGG
CGGCTGAACGCCGTGCTGCTGCCCAACATCCACACCCTGTTCGACATGAGCGCCG
AGGACTTCGACGCCATCATCGCCGAGCACTTCCAGCCCGGCGACTGCGTGCTGG
AGACCGACATCGCCAGCTTCGACAAGAGCGAGGATGACGCTATGGCCCTGACCG
CTCTGATGATCCTGGAGGACCTGGGCGTGGACGCCGAGCTGCTCACCCTGATCGA
GGCTGCCTTCGGCGAGATCAGCTCCATCCACCTGCCCACCAAGACCAAGTTCAAG
TTCGGCGCTATGATGAAAAGCGGAATGTTCCTGACCCTGTTCGTGAACACCGTGA
TCAACATTGTGATCGCCAGCAGGGTGCTGCGGGAGAGGCTGACCGGCAGCCCCT
GCGCTGCCTTCATCGGCGACGACAACATCGTGAAGGGCGTGAAAAGCGACAAGC
TGATGGCCGACAGGTGCGCCACCTGGCTGAACATGGAGGTGAAGATCATCGACG
CCGTGGTGGGCGAGAAGGCCCCCTACTTCTGCGGCGGATTCATCCTGTGCGACAG
CGTGACCGGCACCGCCTGCAGGGTGGCCGACCCCCTGAAGAGGCTGTTCAAGCT
GGGCAAGCCACTGGCCGCTGACGATGAGCACGACGATGACAGGCGGAGGGCCCT
GCACGAGGAAAGCACCAGGTGGAACAGGGTGGGCATCCTGAGCGAGCTGTGCA
AGGCCGTGGAGAGCAGGTACGAGACCGTGGGCACCAGCATCATCGTGATGGCTA
TGACCACACTGGCCAGCTCCGTCAAGAGCTTCTCCTACCTGAGGGGGGCCCCTAT
AACTCTCTACGGCTAACCTGAATGGACTACGACATAGTCTAGTCCGCCAAGGCCG
CCACCATGTTTGTTTTTCTTGTTTTATTGCCACTAGTCTCTAGTCAGTGTGTTAATC
TTACAACCAGAACTCAATTACCCCCTGCATACACTAATTCTTTCACACGTGGTGT
TTATTACCCTGACAAAGTTTTCAGATCCTCAGTTTTACATTCAACTCAGGACTTGT
TCTTACCTTTCTTTTCCAATGTTACTTGGTTCCATGCTATACATGTCTCTGGGACC
AATGGTACTAAGAGGTTTGATAACCCTGTCCTACCATTTAATGATGGTGTTTATTT
TGCTTCCACTGAGAAGTCTAACATAATAAGAGGCTGGATTTTTGGTACTACTTTA
GATTCGAAGACCCAGTCCCTACTTATTGTTAATAACGCTACTAATGTTGTTATTAA
AGTCTGTGAATTTCAATTTTGTAATGATCCATTTTTGGGTGTTTATTACCACAAAA
ACAACAAAAGTTGGATGGAAAGTGAGTTCAGAGTTTATTCTAGTGCGAATAATT
GCACTTTTGAATATGTCTCTCAGCCTTTTCTTATGGACCTTGAAGGAAAACAGGG
TAATTTCAAAAATCTTAGGGAATTTGTGTTTAAGAATATTGATGGTTATTTTAAA
ATATATTCTAAGCACACGCCTATTAATTTAGTGCGTGATCTCCCTCAGGGTTTTTC
GGCTTTAGAACCATTGGTAGATTTGCCAATAGGTATTAACATCACTAGGTTTCAA
ACTTTACTTGCTTTACATAGAAGTTATTTGACTCCTGGTGATTCTTCTTCAGGTTG
GACAGCTGGTGCTGCAGCTTATTATGTGGGTTATCTTCAACCTAGGACTTTTCTAT
TAAAATATAATGAAAATGGAACCATTACAGATGCTGTAGACTGTGCACTTGACC
CTCTCTCAGAAACAAAGTGTACGTTGAAATCCTTCACTGTAGAAAAAGGAATCTA
TCAAACTTCTAACTTTAGAGTCCAACCAACAGAATCTATTGTTAGATTTCCTAAT
ATTACAAACTTGTGCCCTTTTGGTGAAGTTTTTAACGCCACCAGATTTGCATCTGT
TTATGCTTGGAACAGGAAGAGAATCAGCAACTGTGTTGCTGATTATTCTGTCCTA
TATAATTCCGCATCATTTTCCACTTTTAAGTGTTATGGAGTGTCTCCTACTAAATT
AAATGATCTCTGCTTTACTAATGTCTATGCAGATTCATTTGTAATTAGAGGTGATG
AAGTCAGACAAATCGCTCCAGGGCAAACTGGAAAGATTGCTGATTATAATTATA
AATTACCAGATGATTTTACAGGCTGCGTTATAGCTTGGAATTCTAACAATCTTGA
TTCTAAGGTTGGTGGTAATTATAATTACCTGTATAGATTGTTTAGGAAGTCTAATC
TCAAACCTTTTGAGAGAGATATTTCAACTGAAATCTATCAGGCCGGTAGCACACC
TTGTAATGGTGTTGAAGGTTTTAATTGTTACTTTCCTTTACAATCATATGGTTTCC
AACCCACTAATGGTGTTGGTTACCAACCATACAGAGTAGTAGTACTTTCTTTTGA
ACTTCTACATGCACCAGCAACTGTTTGTGGACCTAAAAAGTCTACTAATTTGGTT
AAAAACAAATGTGTCAATTTCAACTTCAATGGTTTAACAGGCACAGGTGTTCTTA
CTGAGTCTAACAAAAAGTTTCTGCCTTTCCAACAATTTGGCAGAGACATTGCTGA
CACTACTGATGCTGTCCGTGATCCACAGACACTTGAGATTCTTGACATTACACCA
TGTTCTTTTGGTGGTGTCAGTGTTATAACACCAGGAACAAATACTTCTAACCAGG
TTGCTGTTCTTTATCAGGATGTTAACTGCACAGAAGTCCCTGTTGCTATTCATGCA
GATCAACTTACTCCTACTTGGCGTGTTTATTCTACAGGTTCTAATGTTTTTCAAAC
ACGTGCAGGCTGTTTAATAGGGGCTGAACATGTCAACAACTCATATGAGTGTGA
CATACCCATTGGTGCAGGTATATGCGCTAGTTATCAGACTCAGACTAATTCTCCT
CGGCGGGCACGTAGTGTAGCTAGTCAATCCATCATTGCCTACACTATGTCACTTG
GTGCAGAAAATTCAGTTGCTTACTCTAATAACTCTATTGCCATACCCACAAATTT
TACTATTAGTGTTACCACAGAAATTCTACCAGTGTCTATGACCAAGACATCAGTA
GATTGTACAATGTACATTTGTGGTGATTCAACTGAATGCAGCAATCTTTTGTTGC
AATATGGCAGTTTTTGTACACAATTAAACCGTGCTTTAACTGGAATAGCTGTTGA
ACAAGACAAAAACACCCAAGAAGTTTTTGCACAAGTCAAACAAATTTACAAAAC
ACCACCAATTAAAGATTTTGGTGGTTTTAATTTTTCACAAATATTACCAGATCCAT
CAAAACCAAGCAAGAGGTCATTTATTGAAGATCTACTTTTCAACAAAGTGACACT
TGCAGATGCTGGCTTCATCAAACAATATGGTGATTGCCTTGGTGATATTGCTGCT
AGAGACCTCATTTGTGCACAAAAGTTTAACGGCCTTACTGTTTTGCCACCTTTGCT
CACAGATGAAATGATTGCTCAATACACTTCTGCACTGTTAGCGGGTACAATCACT
TCTGGTTGGACCTTTGGTGCAGGTGCTGCATTACAAATACCATTTGCTATGCAAA
TGGCTTATAGGTTTAATGGTATTGGAGTTACACAGAATGTTCTCTATGAGAACCA
AAAATTGATTGCCAACCAATTTAATAGTGCTATTGGCAAAATTCAAGACTCACTT
TCTTCCACAGCAAGTGCACTTGGAAAACTTCAAGATGTGGTCAACCAAAATGCA
CAAGCTTTAAACACGCTTGTTAAACAACTTAGCTCCAATTTTGGTGCAATTTCAA
GTGTTTTAAATGATATCCTTTCACGTCTTGACAAAGTTGAGGCTGAAGTGCAAAT
TGATAGGTTGATCACAGGCAGACTTCAAAGTTTGCAGACATATGTGACTCAACA
ATTAATTAGAGCTGCAGAAATCAGAGCTTCTGCTAATCTTGCTGCTACTAAAATG
TCAGAGTGTGTACTTGGACAATCAAAAAGAGTTGATTTTTGTGGAAAGGGCTATC
ATCTTATGTCCTTCCCTCAGTCAGCACCTCATGGTGTAGTCTTCTTGCATGTGACT
TATGTCCCTGCACAAGAAAAGAACTTCACAACTGCTCCTGCCATTTGTCATGATG
GAAAAGCACACTTTCCTCGTGAAGGTGTCTTTGTTTCAAATGGCACACACTGGTT
TGTAACACAAAGGAATTTTTATGAACCACAAATCATTACTACAGACAACACATTT
GTGTCTGGTAACTGTGATGTTGTAATAGGAATTGTCAACAACACAGTTTATGATC
CTTTGCAACCTGAATTAGACTCATTCAAGGAGGAGTTAGATAAATATTTTAAGAA
TCATACATCACCAGATGTTGATTTAGGTGACATCTCTGGCATTAATGCTTCAGTTG
TAAACATTCAAAAAGAAATTGACCGCCTCAATGAGGTTGCCAAGAATTTAAATG
AATCTCTCATCGATCTCCAAGAACTTGGAAAGTATGAGCAGTATATAAAATGGCC
ATGGTACATTTGGCTAGGTTTTATAGCTGGCTTGATTGCCATAGTAATGGTGACA
ATTATGCTTTGCTGTATGACCAGTTGCTGTAGTTGTCTCAAGGGCTGTTGTTCTTG
TGGATCCTGCTGCAAATTTGATGAAGACGACTCTGAGCCAGTGCTCAAAGGAGT
CAAATTACATTACACATAAACTCGAGTATGTTACGTGCAAAGGTGATTGTCACCC
CCCGAAAGACCATATTGTGACACACCCTCAGTATCACGCCCAAACATTTACAGCC
GCGGTGTCAAAAACCGCGTGGACGTGGTTAACATCCCTGCTGGGAGGATCAGCC
GTAATTATTATAATTGGCTTGGTGCTGGCTACTATTGTGGCCATGTACGTGCTGAC
CAACCAGAAACATAATTGAATACAGCAGCAATTGGCAAGCTGCTTACATAGAAC
TCGCGGCGATTGGCATGCCGCCTTAAAATTTTTATTTTATTTTTTCTTTTCTTTTCC
GAATCGGATTTTGTTTTTAATATTTCAAAAAAAAAAAAAAAAAAAAAAAAATCT
AGAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAA
SEQ ID NO: 125
ATGGGCGGCGCATGAGAGAAGCCCAGACCAATTACCTACCCAAAATGGAGAAA
GTTCACGTTGACATCGAGGAAGACAGCCCATTCCTCAGAGCTTTGCAGCGGAGCT
TCCCGCAGTTTGAGGTAGAAGCCAAGCAGGTCACTGATAATGACCATGCTAATG
CCAGAGCGTTTTCGCATCTGGCTTCAAAACTGATCGAAACGGAGGTGGACCCATC
CGACACGATCCTTGACATTGGAAGTGCGCCCGCCCGCAGAATGTATTCTAAGCAC
AAGTATCATTGTATCTGTCCGATGAGATGTGCGGAAGATCCGGACAGATTGTATA
AGTATGCAACTAAGCTGAAGAAAAACTGTAAGGAAATAACTGATAAGGAATTGG
ACAAGAAAATGAAGGAGCTGGCCGCCGTCATGAGCGACCCTGACCTGGAAACTG
AGACTATGTGCCTCCACGACGACGAGTCGTGTCGCTACGAAGGGCAAGTCGCTG
TTTACCAGGATGTATACGCCGTCGACGGCCCCACCAGCCTGTACCACCAGGCCAA
CAAGGGCGTGAGGGTGGCCTACTGGATCGGCTTCGACACCACACCCTTCATGTTC
AAGAACCTGGCCGGCGCCTACCCCAGCTACAGCACCAACTGGGCCGACGAGACC
GTGCTGACCGCCAGGAACATCGGCCTGTGCAGCAGCGACGTGATGGAGAGGAGC
CGGAGAGGCATGAGCATCCTGAGGAAGAAATACCTGAAGCCCAGCAACAACGT
GCTGTTCAGCGTGGGCAGCACCATCTACCACGAGAAGAGGGACCTGCTCAGGAG
CTGGCACCTGCCCAGCGTGTTCCACCTGAGGGGCAAGCAGAACTACACCTGCAG
GTGCGAGACCATCGTGAGCTGCGACGGCTACGTGGTGAAGAGGATCGCCATCAG
CCCCGGCCTGTACGGCAAGCCCAGCGGCTACGCCGCTACAATGCACAGGGAGGG
CTTCCTGTGCTGCAAGGTGACCGACACCCTGAACGGCGAGAGGGTGAGCTTCCC
CGTGTGCACCTACGTGCCCGCCACCCTGTGCGACCAGATGACCGGCATCCTGGCC
ACCGACGTGAGCGCCGACGACGCCCAGAAGCTGCTCGTGGGCCTGAACCAGAGG
ATCGTGGTCAACGGCAGGACCCAGAGGAACACCAACACAATGAAGAACTACCTG
CTGCCCGTGGTGGCCCAGGCTTTCGCCAGGTGGGCCAAGGAGTACAAGGAGGAC
CAGGAAGACGAGAGGCCCCTGGGCCTGAGGGACAGGCAGCTGGTGATGGGCTG
CTGCTGGGCCTTCAGGCGGCACAAGATCACCAGCATCTACAAGAGGCCCGACAC
CCAGACCATCATCAAGGTGAACAGCGACTTCCACAGCTTCGTGCTGCCCAGGATC
GGCAGCAACACCCTGGAGATCGGCCTGAGGACCCGGATCAGGAAGATGCTGGAG
GAACACAAGGAGCCCAGCCCACTGATCACCGCCGAGGACGTGCAGGAGGCCAA
GTGCGCTGCCGACGAGGCCAAGGAGGTGAGGGAGGCCGAGGAACTGAGGGCCG
CCCTGCCACCCCTGGCTGCCGACGTGGAGGAACCCACCCTGGAAGCCGACGTGG
ACCTGATGCTGCAGGAGGCCGGCGCCGGAAGCGTGGAGACACCCAGGGGCCTGA
TCAAGGTGACCAGCTACGACGGCGAGGACAAGATCGGCAGCTACGCCGTGCTGA
GCCCACAGGCCGTGCTGAAGTCCGAGAAGCTGAGCTGCATCCACCCACTGGCCG
AGCAGGTGATCGTGATCACCCACAGCGGCAGGAAGGGCAGGTACGCCGTGGAGC
CCTACCACGGCAAGGTGGTCGTGCCCGAGGGCCACGCCATCCCCGTGCAGGACT
TCCAGGCCCTGAGCGAGAGCGCCACCATCGTGTACAACGAGAGGGAGTTCGTGA
ACAGGTACCTGCACCATATCGCCACCCACGGCGGAGCCCTGAACACCGACGAGG
AATACTACAAGACCGTGAAGCCCAGCGAGCACGACGGCGAGTACCTGTACGACA
TCGACAGGAAGCAGTGCGTGAAGAAAGAGCTGGTGACCGGCCTGGGACTGACCG
GCGAGCTGGTGGACCCACCCTTCCACGAGTTCGCCTACGAGAGCCTGAGGACCA
GACCCGCCGCTCCCTACCAGGTGCCCACCATCGGCGTGTACGGCGTGCCCGGCA
GCGGAAAGAGCGGCATCATCAAGAGCGCCGTGACCAAGAAAGACCTGGTGGTC
AGCGCCAAGAAAGAGAACTGCGCCGAGATCATCAGGGACGTGAAGAAGATGAA
AGGCCTGGACGTGAACGCGCGCACCGTGGACAGCGTGCTGCTGAACGGCTGCAA
GCACCCCGTGGAGACCCTGTACATCGACGAGGCCTTCGCTTGCCACGCCGGCACC
CTGAGGGCCCTGATCGCCATCATCAGGCCCAAGAAAGCCGTGCTGTGCGGCGAC
CCCAAGCAGTGCGGCTTCTTCAACATGATGTGCCTGAAGGTGCACTTCAACCACG
AGATCTGCACCCAGGTGTTCCACAAGAGCATCAGCAGGCGGTGCACCAAGAGCG
TGACCAGCGTCGTGAGCACCCTGTTCTACGACAAGAAAATGAGGACCACCAACC
CCAAGGAGACCAAAATCGTGATCGACACCACAGGCAGCACCAAGCCCAAGCAG
GACGACCTGATCCTGACCTGCTTCAGGGGCTGGGTGAAGCAGCTGCAGATCGAC
TACAAGGGCAACGAGATCATGACCGCCGCTGCCAGCCAGGGCCTGACCAGGAAG
GGCGTGTACGCCGTGAGGTACAAGGTGAACGAGAACCCACTGTACGCTCCCACC
AGCGAGCACGTGAACGTGCTGCTGACCAGGACCGAGGACAGGATCGTGTGGAAG
ACCCTGGCCGGCGACCCCTGGATCAAGACCCTGACCGCCAAGTACCCCGGCAAC
TTCACCGCCACCATCGAAGAGTGGCAGGCCGAGCACGACGCCATCATGAGGCAC
ATCCTGGAGAGGCCCGACCCCACCGACGTGTTCCAGAACAAGGCCAACGTGTGC
TGGGCCAAGGCCCTGGTGCCCGTGCTGAAGACCGCCGGCATCGACATGACCACA
GAGCAGTGGAACACCGTGGACTACTTCGAGACCGACAAGGCCCACAGCGCCGAG
ATCGTGCTGAACCAGCTGTGCGTGAGGTTCTTCGGCCTGGACCTGGACAGCGGCC
TGTTCAGCGCCCCCACCGTGCCACTGAGCATCAGGAACAACCACTGGGACAACA
GCCCCAGCCCAAACATGTACGGCCTGAACAAGGAGGTGGTCAGGCAGCTGAGCA
GGCGGTACCCACAGCTGCCCAGGGCCGTGGCCACCGGCAGGGTGTACGACATGA
ACACCGGCACCCTGAGGAACTACGACCCCAGGATCAACCTGGTGCCCGTGAACA
GGCGGCTGCCCCACGCCCTGGTGCTGCACCACAACGAGCACCCACAGAGCGACT
TCAGCTCCTTCGTGAGCAAGCTGAAAGGCAGGACCGTGCTGGTCGTGGGCGAGA
AGCTGAGCGTGCCCGGCAAGATGGTGGACTGGCTGAGCGACAGGCCCGAGGCCA
CCTTCCGGGCCAGGCTGGACCTCGGCATCCCCGGCGACGTGCCCAAGTACGACA
TCATCTTCGTGAACGTCAGGACCCCATACAAGTACCACCATTACCAGCAGTGCGA
GGACCACGCCATCAAGCTGAGCATGCTGACCAAGAAGGCCTGCCTGCACCTGAA
CCCCGGAGGCACCTGCGTGAGCATCGGCTACGGCTACGCCGACAGGGCCAGCGA
GAGCATCATTGGCGCCATCGCCAGGCTGTTCAAGTTCAGCAGGGTGTGCAAACC
CAAGAGCAGCCTGGAGGAAACCGAGGTGCTGTTCGTGTTCATCGGCTACGACCG
GAAGGCCAGGACCCACAACCCCTACAAGCTGAGCAGCACCCTGACAAACATCTA
CACCGGCAGCAGGCTGCACGAGGCCGGCTGCGCCCCCAGCTACCACGTGGTCAG
GGGCGATATCGCCACCGCCACCGAGGGCGTGATCATCAACGCTGCCAACAGCAA
GGGCCAGCCCGGAGGCGGAGTGTGCGGCGCCCTGTACAAGAAGTTCCCCGAGAG
CTTCGACCTGCAGCCCATCGAGGTGGGCAAGGCCAGGCTGGTGAAGGGCGCCGC
TAAGCACATCATCCACGCCGTGGGCCCCAACTTCAACAAGGTGAGCGAGGTGGA
AGGCGACAAGCAGCTGGCCGAAGCCTACGAGAGCATCGCCAAGATCGTGAACG
ACAATAACTACAAGAGCGTGGCCATCCCACTGCTCAGCACCGGCATCTTCAGCG
GCAACAAGGACAGGCTGACCCAGAGCCTGAACCACCTGCTCACCGCCCTGGACA
CCACCGATGCCGACGTGGCCATCTACTGCAGGGACAAGAAGTGGGAGATGACCC
TGAAGGAGGCCGTGGCCAGGCGGGAGGCCGTGGAAGAGATCTGCATCAGCGAC
GACTCCAGCGTGACCGAGCCCGACGCCGAGCTGGTGAGGGTGCACCCCAAGAGC
TCCCTGGCCGGCAGGAAGGGCTACAGCACCAGCGACGGCAAGACCTTCAGCTAC
CTGGAGGGCACCAAGTTCCACCAGGCCGCTAAGGACATCGCCGAGATCAACGCT
ATGTGGCCCGTGGCCACCGAGGCCAACGAGCAGGTGTGCATGTACATCCTGGGC
GAGAGCATGTCCAGCATCAGGAGCAAGTGCCCCGTGGAGGAAAGCGAGGCCAG
CACACCACCCAGCACCCTGCCCTGCCTGTGCATCCACGCTATGACACCCGAGAGG
GTGCAGCGGCTGAAGGCCAGCAGGCCCGAGCAGATCACCGTGTGCAGCTCCTTC
CCACTGCCCAAGTACAGGATCACCGGCGTGCAGAAGATCCAGTGCAGCCAGCCC
ATCCTGTTCAGCCCAAAGGTGCCCGCCTACATCCACCCCAGGAAGTACCTGGTGG
AGACCCCACCCGTGGACGAGACACCCGAGCCAAGCGCCGAGAACCAGAGCACC
GAGGGCACACCCGAGCAGCCACCCCTGATCACCGAGGACGAGACAAGGACCCG
GACCCCAGAGCCCATCATTATCGAGGAAGAGGAAGAGGACAGCATCAGCCTGCT
GAGCGACGGCCCCACCCACCAGGTGCTGCAGGTGGAGGCCGACATCCACGGCCC
ACCCAGCGTGTCCAGCTCCAGCTGGAGCATCCCACACGCCAGCGACTTCGACGT
GGACAGCCTGAGCATCCTGGACACCCTGGAGGGCGCCAGCGTGACCTCCGGCGC
CACCAGCGCCGAGACCAACAGCTACTTCGCCAAGAGCATGGAGTTCCTGGCCAG
GCCCGTGCCAGCTCCCAGGACCGTGTTCAGGAACCCACCCCACCCAGCTCCCAG
GACCAGGACCCCAAGCCTGGCTCCCAGCAGGGCCTGCAGCAGGACCAGCCTGGT
GAGCACCCCACCCGGCGTGAACAGGGTGATCACCAGGGAGGAACTGGAGGCCCT
GACACCCAGCAGGACCCCCAGCAGGTCCGTGAGCAGGACTAGTCTGGTGTCCAA
CCCACCCGGCGTGAACAGGGTGATCACCAGGGAGGAATTCGAGGCCTTCGTGGC
CCAGCAACAGAGACGGTTCGACGCCGGCGCCTACATCTTCAGCAGCGACACCGG
CCAGGGACACCTGCAGCAAAAGAGCGTGAGGCAGACCGTGCTGAGCGAGGTGG
TGCTGGAGAGGACCGAGCTGGAAATCAGCTACGCCCCCAGGCTGGACCAGGAGA
AGGAGGAACTGCTCAGGAAGAAACTGCAGCTGAACCCCACCCCAGCCAACAGG
AGCAGGTACCAGAGCAGGAAGGTGGAGAACATGAAGGCCATCACCGCCAGGCG
GATCCTGCAGGGCCTGGGACACTACCTGAAGGCCGAGGGCAAGGTGGAGTGCTA
CAGGACCCTGCACCCCGTGCCACTGTACAGCTCCAGCGTGAACAGGGCCTTCTCC
AGCCCCAAGGTGGCCGTGGAGGCCTGCAACGCTATGCTGAAGGAGAACTTCCCC
ACCGTGGCCAGCTACTGCATCATCCCCGAGTACGACGCCTACCTGGACATGGTGG
ACGGCGCCAGCTGCTGCCTGGACACCGCCAGCTTCTGCCCCGCCAAGCTGAGGA
GCTTCCCCAAGAAACACAGCTACCTGGAGCCCACCATCAGGAGCGCCGTGCCCA
GCGCCATCCAGAACACCCTGCAGAACGTGCTGGCCGCTGCCACCAAGAGGAACT
GCAACGTGACCCAGATGAGGGAGCTGCCCGTGCTGGACAGCGCTGCCTTCAACG
TGGAGTGCTTCAAGAAATACGCCTGCAACAACGAGTACTGGGAGACCTTCAAGG
AGAACCCCATCAGGCTGACCGAAGAGAACGTGGTGAACTACATCACCAAGCTGA
AGGGCCCCAAGGCCGCTGCCCTGTTCGCTAAGACCCACAACCTGAACATGCTGC
AGGACATCCCAATGGACAGGTTCGTGATGGACCTGAAGAGGGACGTGAAGGTGA
CACCCGGCACCAAGCACACCGAGGAGAGGCCCAAGGTGCAGGTGATCCAGGCC
GCTGACCCACTGGCCACCGCCTACCTGTGCGGCATCCACAGGGAGCTGGTGAGG
CGGCTGAACGCCGTGCTGCTGCCCAACATCCACACCCTGTTCGACATGAGCGCCG
AGGACTTCGACGCCATCATCGCCGAGCACTTCCAGCCCGGCGACTGCGTGCTGG
AGACCGACATCGCCAGCTTCGACAAGAGCGAGGATGACGCTATGGCCCTGACCG
CTCTGATGATCCTGGAGGACCTGGGCGTGGACGCCGAGCTGCTCACCCTGATCGA
GGCTGCCTTCGGCGAGATCAGCTCCATCCACCTGCCCACCAAGACCAAGTTCAAG
TTCGGCGCTATGATGAAAAGCGGAATGTTCCTGACCCTGTTCGTGAACACCGTGA
TCAACATTGTGATCGCCAGCAGGGTGCTGCGGGAGAGGCTGACCGGCAGCCCCT
GCGCTGCCTTCATCGGCGACGACAACATCGTGAAGGGCGTGAAAAGCGACAAGC
TGATGGCCGACAGGTGCGCCACCTGGCTGAACATGGAGGTGAAGATCATCGACG
CCGTGGTGGGCGAGAAGGCCCCCTACTTCTGCGGCGGATTCATCCTGTGCGACAG
CGTGACCGGCACCGCCTGCAGGGTGGCCGACCCCCTGAAGAGGCTGTTCAAGCT
GGGCAAGCCACTGGCCGCTGACGATGAGCACGACGATGACAGGCGGAGGGCCCT
GCACGAGGAAAGCACCAGGTGGAACAGGGTGGGCATCCTGAGCGAGCTGTGCA
AGGCCGTGGAGAGCAGGTACGAGACCGTGGGCACCAGCATCATCGTGATGGCTA
TGACCACACTGGCCAGCTCCGTCAAGAGCTTCTCCTACCTGAGGGGGGCCCCTAT
AACTCTCTACGGCTAACCTGAATGGACTACGACATAGTCTAGTCCGCCAAGGCCG
CCACCATGTTCGTCTTCCTGGTCCTGCTGCCTCTGGTCTCCTCACAGTGCGTCAAT
CTGACAACTCGGACTCAGCTGCCACCTGCTTATACTAATAGCTTCACCAGAGGCG
TGTACTATCCTGACAAGGTGTTTAGAAGCTCCGTGCTGCACTCTACACAGGATCT
GTTTCTGCCATTCTTTAGCAACGTGACCTGGTTCCACGCCATCCACGTGAGCGGC
ACCAATGGCACAAAGCGGTTCGACAATCCCGTGCTGCCTTTTAACGATGGCGTGT
ACTTCGCCTCTACCGAGAAGTCCAACATCATCAGAGGCTGGATCTTTGGCACCAC
ACTGGACTCCAAGACACAGTCTCTGCTGATCGTGAACAATGCCACCAACGTGGTC
ATCAAGGTGTGCGAGTTCCAGTTTTGTAATGATCCCTTCCTGGGCGTGTACTATC
ACAAGAACAATAAGAGCTGGATGGAGTCCGAGTTTAGAGTGTATTCTAGCGCCA
ACAACTGCACATTTGAGTACGTGAGCCAGCCTTTCCTGATGGACCTGGAGGGCA
AGCAGGGCAATTTCAAGAACCTGAGGGAGTTCGTGTTTAAGAATATCGACGGCT
ACTTCAAAATCTACTCTAAGCACACCCCCATCAACCTGGTGCGCGACCTGCCTCA
GGGCTTCAGCGCCCTGGAGCCCCTGGTGGATCTGCCTATCGGCATCAACATCACC
CGGTTTCAGACACTGCTGGCCCTGCACAGAAGCTACCTGACACCCGGCGACTCCT
CTAGCGGATGGACCGCCGGCGCTGCCGCCTACTATGTGGGCTACCTCCAGCCCCG
GACCTTCCTGCTGAAGTACAACGAGAATGGCACCATCACAGACGCAGTGGATTG
CGCCCTGGACCCCCTGAGCGAGACAAAGTGTACACTGAAGTCCTTTACCGTGGA
GAAGGGCATCTATCAGACATCCAATTTCAGGGTGCAGCCAACCGAGTCTATCGT
GCGCTTTCCTAATATCACAAACCTGTGCCCATTTGGCGAGGTGTTCAACGCAACC
CGCTTCGCCAGCGTGTACGCCTGGAATAGGAAGCGGATCAGCAACTGCGTGGCC
GACTATAGCGTGCTGTACAACTCCGCCTCTTTCAGCACCTTTAAGTGCTATGGCG
TGTCCCCCACAAAGCTGAATGACCTGTGCTTTACCAACGTCTACGCCGATTCTTT
CGTGATCAGGGGCGACGAGGTGCGCCAGATCGCCCCCGGCCAGACAGGCAAGAT
CGCAGACTACAATTATAAGCTGCCAGACGATTTCACCGGCTGCGTGATCGCCTGG
AACAGCAACAATCTGGATTCCAAAGTGGGCGGCAACTACAATTATCTGTACCGG
CTGTTTAGAAAGAGCAATCTGAAGCCCTTCGAGAGGGACATCTCTACAGAAATC
TACCAGGCCGGCAGCACCCCTTGCAATGGCGTGGAGGGCTTTAACTGTTATTTCC
CACTCCAGTCCTACGGCTTCCAGCCCACAAACGGCGTGGGCTATCAGCCTTACCG
CGTGGTGGTGCTGAGCTTTGAGCTGCTGCACGCCCCAGCAACAGTGTGCGGCCCC
AAGAAGTCCACCAATCTGGTGAAGAACAAGTGCGTGAACTTCAACTTCAACGGC
CTGACCGGCACAGGCGTGCTGACCGAGTCCAACAAGAAGTTCCTGCCATTTCAG
CAGTTCGGCAGGGACATCGCAGATACCACAGACGCCGTGCGCGACCCACAGACC
CTGGAGATCCTGGACATCACACCCTGCTCTTTCGGCGGCGTGAGCGTGATCACAC
CCGGCACCAATACAAGCAACCAGGTGGCCGTGCTGTATCAGGACGTGAATTGTA
CCGAGGTGCCCGTGGCTATCCACGCCGATCAGCTGACCCCAACATGGCGGGTGT
ACAGCACCGGCTCCAACGTCTTCCAGACAAGAGCCGGATGCCTGATCGGAGCAG
AGCACGTGAACAATTCCTATGAGTGCGACATCCCAATCGGCGCCGGCATCTGTGC
CTCTTACCAGACCCAGACAAACTCTCCCAGACGGGCCCGGAGCGTGGCCTCCCA
GTCTATCATCGCCTATACCATGTCCCTGGGCGCCGAGAACAGCGTGGCCTACTCT
AACAATAGCATCGCCATCCCAACCAACTTCACAATCTCTGTGACCACAGAGATCC
TGCCCGTGTCCATGACCAAGACATCTGTGGACTGCACAATGTATATCTGTGGCGA
TTCTACCGAGTGCAGCAACCTGCTGCTCCAGTACGGCAGCTTTTGTACCCAGCTG
AATAGAGCCCTGACAGGCATCGCCGTGGAGCAGGATAAGAACACACAGGAGGT
GTTCGCCCAGGTGAAGCAAATCTACAAGACCCCCCCTATCAAGGACTTTGGCGG
CTTCAATTTTTCCCAGATCCTGCCTGATCCATCCAAGCCTTCTAAGCGGAGCTTTA
TCGAGGACCTGCTGTTCAACAAGGTGACCCTGGCCGATGCCGGCTTCATCAAGCA
GTATGGCGATTGCCTGGGCGACATCGCAGCCAGGGACCTGATCTGCGCCCAGAA
GTTTAATGGCCTGACCGTGCTGCCACCCCTGCTGACAGATGAGATGATCGCACAG
TACACAAGCGCCCTGCTGGCCGGCACCATCACATCCGGATGGACCTTCGGCGCA
GGAGCCGCCCTCCAGATCCCCTTTGCCATGCAGATGGCCTATAGGTTCAACGGCA
TCGGCGTGACCCAGAATGTGCTGTACGAGAACCAGAAGCTGATCGCCAATCAGT
TTAACTCCGCCATCGGCAAGATCCAGGACAGCCTGTCCTCTACAGCCAGCGCCCT
GGGCAAGCTCCAGGATGTGGTGAATCAGAACGCCCAGGCCCTGAATACCCTGGT
GAAGCAGCTGAGCAGCAACTTCGGCGCCATCTCTAGCGTGCTGAATGACATCCT
GAGCCGGCTGGACAAGGTGGAGGCAGAGGTGCAGATCGACCGGCTGATCACCG
GCCGGCTCCAGAGCCTCCAGACCTATGTGACACAGCAGCTGATCAGGGCCGCCG
AGATCAGGGCCAGCGCCAATCTGGCAGCAACCAAGATGTCCGAGTGCGTGCTGG
GCCAGTCTAAGAGAGTGGACTTTTGTGGCAAGGGCTATCACCTGATGTCCTTCCC
TCAGTCTGCCCCACACGGCGTGGTGTTTCTGCACGTGACCTACGTGCCCGCCCAG
GAGAAGAACTTCACCACAGCCCCTGCCATCTGCCACGATGGCAAGGCCCACTTTC
CAAGGGAGGGCGTGTTCGTGTCCAACGGCACCCACTGGTTTGTGACACAGCGCA
ATTTCTACGAGCCCCAGATCATCACCACAGACAACACCTTCGTGAGCGGCAACTG
TGACGTGGTCATCGGCATCGTGAACAATACCGTGTATGATCCACTCCAGCCCGAG
CTGGACAGCTTTAAGGAGGAGCTGGATAAGTATTTCAAGAATCACACCTCCCCTG
ACGTGGATCTGGGCGACATCAGCGGCATCAATGCCTCCGTGGTGAACATCCAGA
AGGAGATCGACCGCCTGAACGAGGTGGCTAAGAATCTGAACGAGAGCCTGATCG
ACCTCCAGGAGCTGGGCAAGTATGAGCAGTACATCAAGTGGCCCTGGTACATCT
GGCTGGGCTTCATCGCCGGCCTGATCGCCATCGTGATGGTGACCATCATGCTGTG
CTGTATGACATCCTGCTGTTCTTGCCTGAAGGGCTGCTGTAGCTGTGGCTCCTGCT
GTAAGTTTGACGAGGATGACTCTGAACCTGTGCTGAAGGGCGTGAAGCTGCATT
ACACCTAAACTCGAGTATGTTACGTGCAAAGGTGATTGTCACCCCCCGAAAGAC
CATATTGTGACACACCCTCAGTATCACGCCCAAACATTTACAGCCGCGGTGTCAA
AAACCGCGTGGACGTGGTTAACATCCCTGCTGGGAGGATCAGCCGTAATTATTAT
AATTGGCTTGGTGCTGGCTACTATTGTGGCCATGTACGTGCTGACCAACCAGAAA
CATAATTGAATACAGCAGCAATTGGCAAGCTGCTTACATAGAACTCGCGGCGAT
TGGCATGCCGCCTTAAAATTTTTATTTTATTTTTTCTTTTCTTTTCCGAATCGGATT
TTGTTTTTAATATTTCAAAAAAAAAAAAAAAAAAAAAAAAATCTAGAAAAAAAA
AAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAA
SEQ ID NO: 79
MEKVHVDIEEDSPFLRALQRSFPQFEVEAKQVTDNDHANARAFSHLASKLIETEVDP
SDTILDIGSAPARRMYSKHKYHCICPMRCAEDPDRLYKYATKLKKNCKEITDKELDK
KMKELAAVMSDPDLETETMCLHDDESCRYEGQVAVYQDVYAVDGPTSLYHQANK
GVRVAYWIGFDTTPFMFKNLAGAYPSYSTNWADETVLTARNIGLCSSDVMERSRRG
MSILRKKYLKPSNNVLFSVGSTIYHEKRDLLRSWHLPSVFHLRGKQNYTCRCETIVSC
DGYVVKRIAISPGLYGKPSGYAATMHREGFLCCKVTDTLNGERVSFPVCTYVPATLC
DQMTGILATDVSADDAQKLLVGLNQRIVVNGRTQRNTNTMKNYLLPVVAQAFARW
AKEYKEDQEDERPLGLRDRQLVMGCCWAFRRHKITSIYKRPDTQTIIKVNSDFHSFV
LPRIGSNTLEIGLRTRIRKMLEEHKEPSPLITAEDVQEAKCAADEAKEVREAEELRAA
LPPLAADVEEPTLEADVDLMLQEAGAGSVETPRGLIKVTSYDGEDKIGSYAVLSPQA
VLKSEKLSCIHPLAEQVIVITHSGRKGRYAVEPYHGKVVVPEGHAIPVQDFQALSESA
TIVYNEREFVNRYLHHIATHGGALNTDEEYYKTVKPSEHDGEYLYDIDRKQCVKKEL
VTGLGLTGELVDPPFHEFAYESLRTRPAAPYQVPTIGVYGVPGSGKSGIIKSAVTKKD
LVVSAKKENCAEIIRDVKKMKGLDVNARTVDSVLLNGCKHPVETLYIDEAFACHAG
TLRALIAIIRPKKAVLCGDPKQCGFFNMMCLKVHFNHEICTQVFHKSISRRCTKSVTS
VVSTLFYDKKMRTTNPKETKIVIDTTGSTKPKQDDLILTCFRGWVKQLQIDYKGNEI
MTAAASQGLTRKGVYAVRYKVNENPLYAPTSEHVNVLLTRTEDRIVWKTLAGDPW
IKTLTAKYPGNFTATIEEWQAEHDAIMRHILERPDPTDVFQNKANVCWAKALVPVL
KTAGIDMTTEQWNTVDYFETDKAHSAEIVLNQLCVRFFGLDLDSGLFSAPTVPLSIR
NNHWDNSPSPNMYGLNKEVVRQLSRRYPQLPRAVATGRVYDMNTGTLRNYDPRIN
LVPVNRRLPHALVLHHNEHPQSDFSSFVSKLKGRTVLVVGEKLSVPGKMVDWLSDR
PEATFRARLDLGIPGDVPKYDIIFVNVRTPYKYHHYQQCEDHAIKLSMLTKKACLHL
NPGGTCVSIGYGYADRASESIIGAIARLFKFSRVCKPKSSLEETEVLFVFIGYDRKART
HNPYKLSSTLTNIYTGSRLHEAGCAPSYHVVRGDIATATEGVIINAANSKGQPGGGV
CGALYKKFPESFDLQPIEVGKARLVKGAAKHIIHAVGPNFNKVSEVEGDKQLAEAYE
SIAKIVNDNNYKSVAIPLLSTGIFSGNKDRLTQSLNHLLTALDTTDADVAIYCRDKKW
EMTLKEAVARREAVEEICISDDSSVTEPDAELVRVHPKSSLAGRKGYSTSDGKTFSYL
EGTKFHQAAKDIAEINAMWPVATEANEQVCMYILGESMSSIRSKCPVEESEASTPPST
LPCLCIHAMTPERVQRLKASRPEQITVCSSFPLPKYRITGVQKIQCSQPILFSPKVPAYI
HPRKYLVETPPVDETPEPSAENQSTEGTPEQPPLITEDETRTRTPEPIIIEEEEEDSISLLS
DGPTHQVLQVEADIHGPPSVSSSSWSIPHASDFDVDSLSILDTLEGASVTSGATSAETN
SYFAKSMEFLARPVPAPRTVFRNPPHPAPRTRTPSLAPSRACSRTSLVSTPPGVNRVIT
REELEALTPSRTPSRSVSRTSLVSNPPGVNRVITREEFEAFVAQQQRRFDAGAYIFSSD
TGQGHLQQKSVRQTVLSEVVLERTELEISYAPRLDQEKEELLRKKLQLNPTPANRSR
YQSRKVENMKAITARRILQGLGHYLKAEGKVECYRTLHPVPLYSSSVNRAFSSPKVA
VEACNAMLKENFPTVASYCIIPEYDAYLDMVDGASCCLDTASFCPAKLRSFPKKHSY
LEPTIRSAVPSAIQNTLQNVLAAATKRNCNVTQMRELPVLDSAAFNVECFKKYACNN
EYWETFKENPIRLTEENVVNYITKLKGPKAAALFAKTHNLNMLQDIPMDRFVMDLK
RDVKVTPGTKHTEERPKVQVIQAADPLATAYLCGIHRELVRRLNAVLLPNIHTLFDM
SAEDFDAIIAEHFQPGDCVLETDIASFDKSEDDAMALTALMILEDLGVDAELLTLIEA
AFGEISSIHLPTKTKFKFGAMMKSGMFLTLFVNTVINIVIASRVLRERLTGSPCAAFIG
DDNIVKGVKSDKLMADRCATWLNMEVKIIDAVVGEKAPYFCGGFILCDSVTGTACR
VADPLKRLFKLGKPLAADDEHDDDRRRALHEESTRWNRVGILSELCKAVESRYETV
GTSIIVMAMTTLASSVKSFSYLRGAPITLYG
SEQ ID NO: 80
MPEKVHVDIEEDSPFLRALQRSFPQFEVEAKQVTDNDHANARAFSHLASKLIETEVD
PSDTILDIGSAPARRMYSKHKYHCICPMRCAEDPDRLYKYATKLKKNCKEITDKELD
KKMKELAAVMSDPDLETETMCLHDDESCRYEGQVAVYQDVYAVDGPTSLYHQAN
KGVRVAYWIGFDTTPFMFKNLAGAYPSYSTNWADETVLTARNIGLCSSDVMERSRR
GMSILRKKYLKPSNNVLFSVGSTIYHEKRDLLRSWHLPSVFHLRGKQNYTCRCETIVS
CDGYVVKRIAISPGLYGKPSGYAATMHREGFLCCKVTDTLNGERVSFPVCTYVPATL
CDQMTGILATDVSADDAQKLLVGLNQRIVVNGRTQRNTNTMKNYLLPVVAQAFAR
WAKEYKEDQEDERPLGLRDRQLVMGCCWAFRRHKITSIYKRPDTQTIIKVNSDFHSF
VLPRIGSNTLEIGLRTRIRKMLEEHKEPSPLITAEDVQEAKCAADEAKEVREAEELRA
ALPPLAADVEEPTLEADVDLMLQEAGAGSVETPRGLIKVTSYDGEDKIGSYAVLSPQ
AVLKSEKLSCIHPLAEQVIVITHSGRKGRYAVEPYHGKVVVPEGHAIPVQDFQALSES
ATIVYNEREFVNRYLHHIATHGGALNTDEEYYKTVKPSEHDGEYLYDIDRKQCVKK
ELVTGLGLTGELVDPPFHEFAYESLRTRPAAPYQVPTIGVYGVPGSGKSGIIKSAVTK
KDLVVSAKKENCAEIIRDVKKMKGLDVNARTVDSVLLNGCKHPVETLYIDEAFACH
AGTLRALIAIIRPKKAVLCGDPKQCGFFNMMCLKVHFNHEICTQVFHKSISRRCTKSV
TSVVSTLFYDKKMRTTNPKETKIVIDTTGSTKPKQDDLILTCFRGWVKQLQIDYKGN
EIMTAAASQGLTRKGVYAVRYKVNENPLYAPTSEHVNVLLTRTEDRIVWKTLAGDP
WIKTLTAKYPGNFTATIEEWQAEHDAIMRHILERPDPTDVFQNKANVCWAKALVPV
LKTAGIDMTTEQWNTVDYFETDKAHSAEIVLNQLCVRFFGLDLDSGLFSAPTVPLSIR
NNHWDNSPSPNMYGLNKEVVRQLSRRYPQLPRAVATGRVYDMNTGTLRNYDPRIN
LVPVNRRLPHALVLHHNEHPQSDFSSFVSKLKGRTVLVVGEKLSVPGKMVDWLSDR
PEATFRARLDLGIPGDVPKYDIIFVNVRTPYKYHHYQQCEDHAIKLSMLTKKACLHL
NPGGTCVSIGYGYADRASESIIGAIARLFKFSRVCKPKSSLEETEVLFVFIGYDRKART
HNPYKLSSTLTNIYTGSRLHEAGCAPSYHVVRGDIATATEGVIINAANSKGQPGGGV
CGALYKKFPESFDLQPIEVGKARLVKGAAKHIIHAVGPNFNKVSEVEGDKQLAEAYE
SIAKIVNDNNYKSVAIPLLSTGIFSGNKDRLTQSLNHLLTALDTTDADVAIYCRDKKW
EMTLKEAVARREAVEEICISDDSSVTEPDAELVRVHPKSSLAGRKGYSTSDGKTFSYL
EGTKFHQAAKDIAEINAMWPVATEANEQVCMYILGESMSSIRSKCPVEESEASTPPST
LPCLCIHAMTPERVQRLKASRPEQITVCSSFPLPKYRITGVQKIQCSQPILFSPKVPAYI
HPRKYLVETPPVDETPEPSAENQSTEGTPEQPPLITEDETRTRTPEPIIIEEEEEDSISLLS
DGPTHQVLQVEADIHGPPSVSSSSWSIPHASDFDVDSLSILDTLEGASVTSGATSAETN
SYFAKSMEFLARPVPAPRTVFRNPPHPAPRTRTPSLAPSRACSRTSLVSTPPGVNRVIT
REELEALTPSRTPSRSVSRTSLVSNPPGVNRVITREEFEAFVAQQQRRFDAGAYIFSSD
TGQGHLQQKSVRQTVLSEVVLERTELEISYAPRLDQEKEELLRKKLQLNPTPANRSR
YQSRKVENMKAITARRILQGLGHYLKAEGKVECYRTLHPVPLYSSSVNRAFSSPKVA
VEACNAMLKENFPTVASYCIIPEYDAYLDMVDGASCCLDTASFCPAKLRSFPKKHSY
LEPTIRSAVPSAIQNTLQNVLAAATKRNCNVTQMRELPVLDSAAFNVECFKKYACNN
EYWETFKENPIRLTEENVVNYITKLKGPKAAALFAKTHNLNMLQDIPMDRFVMDLK
RDVKVTPGTKHTEERPKVQVIQAADPLATAYLCGIHRELVRRLNAVLLPNIHTLFDM
SAEDFDAIIAEHFQPGDCVLETDIASFDKSEDDAMALTALMILEDLGVDAELLTLIEA
AFGEISSIHLPTKTKFKFGAMMKSGMFLTLFVNTVINIVIASRVLRERLTGSPCAAFIG
DDNIVKGVKSDKLMADRCATWLNMEVKIIDAVVGEKAPYFCGGFILCDSVTGTACR
VADPLKRLFKLGKPLAADDEHDDDRRRALHEESTRWNRVGILSELCKAVESRYETV
GTSIIVMAMTTLASSVKSFSYLRGAPITLYG
SEQ ID NO: 81
MEKVHVDIEEDSPFLRALQRSFPQFEVEAKQVTDNDHANARAFSHLASKLIETEVDP
SDTILDIGSAPARRMYSKHKYHCICPMRCAEDPDRLYKYATKLKKNCKEITDKELDK
KMKELAAVMSDPDLETETMCLHDDESCRYEGQVAVYQDVYAVDGPTSLYHQANK
GVRVAYWIGFDTTPFMFKNLAGAYPSYSTNWADETVLTARNIGLCSSDVMERSRRG
MSILRKKYLKPSNNVLFSVGSTIYHEKRDLLRSWHLPSVFHLRGKQNYTCRCETIVSC
DGYVVKRIAISPGLYGKPSGYAATMHREGFLCCKVTDTLNGERVSFPVCTYVPATLC
DQMTGILATDVSADDAQKLLVGLNQRIVVNGRTQRNTNTMKNYLLPVVAQAFARW
AKEYKEDQEDERPLGLRDRQLVMGCCWAFRRHKITSIYKRPDTQTIIKVNSDFHSFV
LPRIGSNTLEIGLRTRIRKMLEEHKEPSPLITAEDIQEAKCAADEAKEVREAEELRAAL
PPLAADFEEPTLEADVDLMLQEAGAGSVETPRGLIKVTSYAGEDKIGSYAVLSPQAV
LKSEKLSCIHPLAEQVIVITHSGRKGRYAVEPYHGKVVVPEGHAIPVQDFQALSESAT
IVYNEREFVNRYLHHIATHGGALNTDEEYYKTVKPSEHDGEYLYDIDRKQCVKKEL
VTGLGLTGELVDPPFHEFAYESLRTRPAAPYQVPTIGVYGVPGSGKSGIIKSAVTKKD
LVVSAKKENCAEIIRDVKKMKGLDVNARTVDSVLLNGCKHPVETLYIDEAFACHAG
TLRALIAIIRPKKAVLCGDPKQCGFFNMMCLKVHFNHEICTQVFHKSISRRCTKSVTS
VVSTLFYDKRMRTTNPKETKIVIDTTGSTKPKQDDLILTCFRGWVKQLQIDYKGNEI
MTAAASQGLTRKGVYAVRYKVNENPLYAPTSEHVNVLLTRTEDRIVWKTLAGDPW
IKILTAKYPGNFTATIEEWQAEHDAIMRHILERPDPTDVFQNKANVCWAKALVPVLK
TAGIDMTTEQWNTVDYFETDKAHSAEIVLNQLCVRFFGLDLDSGLFSAPTVPLSIRN
NHWDNSPSPNMYGLNKEVVRQLSRRYPQLPRAVATGRVYDMNTGTLRNYDPRINL
VPVNRRLPHALVLHHNEHPQSDFSSFVSKLKGRTVLVVGEKLSVPGKKVDWLSDQP
EATFRARLDLGIPGDVPKYDIVFINVRTPYKYHHYQQCEDHAIKLSMLTKKACLHLN
PGGTCVSIGYGYADRASESIIGAIARQFKFSRVCKPKSSHEETEVLFVFIGYDRKARTH
NPYKLSSTLTNIYTGSRLHEAGCAPSYHVVRGDIATATEGVIINAANSKGQPGGGVC
GALYKKFPESFDLQPIEVGKARLVKGAAKHIIHAVGPNFNKVSEVEGDKQLAEAYES
IAKIVNDNNYKSVAIPLLSTGIFSGNKDRLTQSLNHLLTALDTTDADVAIYCRDKKWE
MTLKEAVARREAVEEICISDDSSVTEPDAELVRVHPKSSLAGRKGYSTSDGKTFSYLE
GTKFHQAAKDIAEINAMWPVATEANEQVCMYILGESMSSIRSKCPVEESEASTPPSTL
PCLCIHAMTPERVQRLKASRPEQITVCSSFPLPKYRITGVQKIQCSQPILFSPKVPAYIH
PRKYLVETPPVEETPESPAENQSTEGTPEQPALVNVDATRTRMPEPIIIEEEEEDSISLL
SDGPTHQVLQVEADIHGSPSVSSSSWSIPHASDFDVDSLSILDTLDGASVTSGAVSAET
NSYFARSMEFRARPVPAPRTVFRNPPHPAPRTRTPPLAHSRASSRTSLVSTPPGVNRVI
TREELEALTPSRAPSRSASRTSLVSNPPGVNRVITREEFEAFVAQQQ*RFDAGAYIFSS
DTGQGHLQQKSVRQTVLSEVVLERTELEISYAPRLDQEKEELLRKKLQLNPTPANRS
RYQSRRVENMKAITARRILQGLGHYLKAEGKVECYRTLHPVPLYSSSVNRAFSSPKV
AVEACNAMLKENFPTVASYCIIPEYDAYLDMVDGASCCLDTASFCPAKLRSFPKKHS
YLEPTIRSAVPSAIQNTLQNVLAAATKRNCNVTQMRELPVLDSAAFNVECFKKYACN
NEYWETFKENPIRLTEENVVNYITKLKGPKAAALFAKTHNLNMLQDIPMDRFVMDL
KRDVKVTPGTKHTEERPKVQVIQAADPLATADLCGIHRELVRRLNAVLLPNIHTLFD
MSAEDFDAIIAEHFQPGDCVLETDIASFDKSEDDAMALTALMILEDLGVDAELLTLIE
AAFGEISSIHLPTKTKFKFGAMMKSGMFLTLFVNTVINIVIASRVLRERLTGSPCAAFI
GDDNIVKGVKSDKLMADRCATWLNMEVKIIDAVVGEKAPYFCGGFILCDSVTGTAC
RVADPLKRLFKLGKPLAVDDEHDDDRRRALHEESTRWNRVGILPELCKAVESRYET
VGTSIIVMAMTTLASSVKSFSYLRGAPITLYG*
SEQ ID NO: 126
AGGAAACTTAAGTCAACACAACATATACAAAACAAACGAATCTCAAGCAATCAA
GCATTCTACTTCTATTGCAGCAATTTAAATCATTTCTTTTAAAGCAAAAGCAATTT
TCTGAAAATTTTCACCATTTACGAACGATAGCCACCATGTTCGTCTTCCTGGTCCT
GCTGCCTCTGGTCTCCTCACAGTGCGTCAATCTGACAACTCGGACTCAGCTGCCA
CCTGCTTATACTAATAGCTTCACCAGAGGCGTGTACTATCCTGACAAGGTGTTTA
GAAGCTCCGTGCTGCACTCTACACAGGATCTGTTTCTGCCATTCTTTAGCAACGT
GACCTGGTTCCACGCCATCCACGTGAGCGGCACCAATGGCACAAAGCGGTTCGA
CAATCCCGTGCTGCCTTTTAACGATGGCGTGTACTTCGCCTCTACCGAGAAGTCC
AACATCATCAGAGGCTGGATCTTTGGCACCACACTGGACTCCAAGACACAGTCTC
TGCTGATCGTGAACAATGCCACCAACGTGGTCATCAAGGTGTGCGAGTTCCAGTT
TTGTAATGATCCCTTCCTGGGCGTGTACTATCACAAGAACAATAAGAGCTGGATG
GAGTCCGAGTTTAGAGTGTATTCTAGCGCCAACAACTGCACATTTGAGTACGTGA
GCCAGCCTTTCCTGATGGACCTGGAGGGCAAGCAGGGCAATTTCAAGAACCTGA
GGGAGTTCGTGTTTAAGAATATCGACGGCTACTTCAAAATCTACTCTAAGCACAC
CCCCATCAACCTGGTGCGCGACCTGCCTCAGGGCTTCAGCGCCCTGGAGCCCCTG
GTGGATCTGCCTATCGGCATCAACATCACCCGGTTTCAGACACTGCTGGCCCTGC
ACAGAAGCTACCTGACACCCGGCGACTCCTCTAGCGGATGGACCGCCGGCGCTG
CCGCCTACTATGTGGGCTACCTCCAGCCCCGGACCTTCCTGCTGAAGTACAACGA
GAATGGCACCATCACAGACGCAGTGGATTGCGCCCTGGACCCCCTGAGCGAGAC
AAAGTGTACACTGAAGTCCTTTACCGTGGAGAAGGGCATCTATCAGACATCCAA
TTTCAGGGTGCAGCCAACCGAGTCTATCGTGCGCTTTCCTAATATCACAAACCTG
TGCCCATTTGGCGAGGTGTTCAACGCAACCCGCTTCGCCAGCGTGTACGCCTGGA
ATAGGAAGCGGATCAGCAACTGCGTGGCCGACTATAGCGTGCTGTACAACTCCG
CCTCTTTCAGCACCTTTAAGTGCTATGGCGTGTCCCCCACAAAGCTGAATGACCT
GTGCTTTACCAACGTCTACGCCGATTCTTTCGTGATCAGGGGCGACGAGGTGCGC
CAGATCGCCCCCGGCCAGACAGGCAAGATCGCAGACTACAATTATAAGCTGCCA
GACGATTTCACCGGCTGCGTGATCGCCTGGAACAGCAACAATCTGGATTCCAAA
GTGGGCGGCAACTACAATTATCTGTACCGGCTGTTTAGAAAGAGCAATCTGAAG
CCCTTCGAGAGGGACATCTCTACAGAAATCTACCAGGCCGGCAGCACCCCTTGC
AATGGCGTGGAGGGCTTTAACTGTTATTTCCCACTCCAGTCCTACGGCTTCCAGC
CCACAAACGGCGTGGGCTATCAGCCTTACCGCGTGGTGGTGCTGAGCTTTGAGCT
GCTGCACGCCCCAGCAACAGTGTGCGGCCCCAAGAAGTCCACCAATCTGGTGAA
GAACAAGTGCGTGAACTTCAACTTCAACGGCCTGACCGGCACAGGCGTGCTGAC
CGAGTCCAACAAGAAGTTCCTGCCATTTCAGCAGTTCGGCAGGGACATCGCAGA
TACCACAGACGCCGTGCGCGACCCACAGACCCTGGAGATCCTGGACATCACACC
CTGCTCTTTCGGCGGCGTGAGCGTGATCACACCCGGCACCAATACAAGCAACCA
GGTGGCCGTGCTGTATCAGGACGTGAATTGTACCGAGGTGCCCGTGGCTATCCAC
GCCGATCAGCTGACCCCAACATGGCGGGTGTACAGCACCGGCTCCAACGTCTTCC
AGACAAGAGCCGGATGCCTGATCGGAGCAGAGCACGTGAACAATTCCTATGAGT
GCGACATCCCAATCGGCGCCGGCATCTGTGCCTCTTACCAGACCCAGACAAACTC
TCCCAGACGGGCCCGGAGCGTGGCCTCCCAGTCTATCATCGCCTATACCATGTCC
CTGGGCGCCGAGAACAGCGTGGCCTACTCTAACAATAGCATCGCCATCCCAACC
AACTTCACAATCTCTGTGACCACAGAGATCCTGCCCGTGTCCATGACCAAGACAT
CTGTGGACTGCACAATGTATATCTGTGGCGATTCTACCGAGTGCAGCAACCTGCT
GCTCCAGTACGGCAGCTTTTGTACCCAGCTGAATAGAGCCCTGACAGGCATCGCC
GTGGAGCAGGATAAGAACACACAGGAGGTGTTCGCCCAGGTGAAGCAAATCTAC
AAGACCCCCCCTATCAAGGACTTTGGCGGCTTCAATTTTTCCCAGATCCTGCCTG
ATCCATCCAAGCCTTCTAAGCGGAGCTTTATCGAGGACCTGCTGTTCAACAAGGT
GACCCTGGCCGATGCCGGCTTCATCAAGCAGTATGGCGATTGCCTGGGCGACATC
GCAGCCAGGGACCTGATCTGCGCCCAGAAGTTTAATGGCCTGACCGTGCTGCCA
CCCCTGCTGACAGATGAGATGATCGCACAGTACACAAGCGCCCTGCTGGCCGGC
ACCATCACATCCGGATGGACCTTCGGCGCAGGAGCCGCCCTCCAGATCCCCTTTG
CCATGCAGATGGCCTATAGGTTCAACGGCATCGGCGTGACCCAGAATGTGCTGT
ACGAGAACCAGAAGCTGATCGCCAATCAGTTTAACTCCGCCATCGGCAAGATCC
AGGACAGCCTGTCCTCTACAGCCAGCGCCCTGGGCAAGCTCCAGGATGTGGTGA
ATCAGAACGCCCAGGCCCTGAATACCCTGGTGAAGCAGCTGAGCAGCAACTTCG
GCGCCATCTCTAGCGTGCTGAATGACATCCTGAGCCGGCTGGACAAGGTGGAGG
CAGAGGTGCAGATCGACCGGCTGATCACCGGCCGGCTCCAGAGCCTCCAGACCT
ATGTGACACAGCAGCTGATCAGGGCCGCCGAGATCAGGGCCAGCGCCAATCTGG
CAGCAACCAAGATGTCCGAGTGCGTGCTGGGCCAGTCTAAGAGAGTGGACTTTT
GTGGCAAGGGCTATCACCTGATGTCCTTCCCTCAGTCTGCCCCACACGGCGTGGT
GTTTCTGCACGTGACCTACGTGCCCGCCCAGGAGAAGAACTTCACCACAGCCCCT
GCCATCTGCCACGATGGCAAGGCCCACTTTCCAAGGGAGGGCGTGTTCGTGTCCA
ACGGCACCCACTGGTTTGTGACACAGCGCAATTTCTACGAGCCCCAGATCATCAC
CACAGACAACACCTTCGTGAGCGGCAACTGTGACGTGGTCATCGGCATCGTGAA
CAATACCGTGTATGATCCACTCCAGCCCGAGCTGGACAGCTTTAAGGAGGAGCT
GGATAAGTATTTCAAGAATCACACCTCCCCTGACGTGGATCTGGGCGACATCAGC
GGCATCAATGCCTCCGTGGTGAACATCCAGAAGGAGATCGACCGCCTGAACGAG
GTGGCTAAGAATCTGAACGAGAGCCTGATCGACCTCCAGGAGCTGGGCAAGTAT
GAGCAGTACATCAAGTGGCCCTGGTACATCTGGCTGGGCTTCATCGCCGGCCTGA
TCGCCATCGTGATGGTGACCATCATGCTGTGCTGTATGACATCCTGCTGTTCTTGC
CTGAAGGGCTGCTGTAGCTGTGGCTCCTGCTGTAAGTTTGACGAGGATGACTCTG
AACCTGTGCTGAAGGGCGTGAAGCTGCATTACACCTAAACTCGAGCTAGTGACT
GACTAGGATCTGGTTACCACTAAACCAGCCTCAAGAACACCCGAATGGAGTCTC
TAAGCTACATAATACCAACTTACACTTACAAAATGTTGTCCCCCAAAATGTAGCC
ATTCGTATCTGCTCCTAATAAAAAGAAAGTTTCTTCACATTCTAGAAAAAAAAAA
AAAAAAAAAAAAAAAAAAAAAAAAAAAAAAAAAAAAAAAAAAAAAAAAAAAA
AAAAAAAAAAAAAAAAAAAAAAAAAAAAAAAAAAAAAA
SEQ ID NO: 82
AGGAAACTTAAGTCAACACAACATATACAAAACAAACGAATCTCAAGCAATCAA
GCATTCTACTTCTATTGCAGCAATTTAAATCATTTCTTTTAAAGCAAAAGCAATTT
TCTGAAAATTTTCACCATTTACGAACGATAGCCACC
SEQ ID NO: 83
ACTCGAGCTAGTGACTGACTAGGATCTGGTTACCACTAAACCAGCCTCAAGAAC
ACCCGAATGGAGTCTCTAAGCTACATAATACCAACTTACACTTACAAAATGTTGT
CCCCCAAAATGTAGCCATTCGTATCTGCTCCTAATAAAAAGAAAGTTTCTTCACA
TTCTAGAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAA
AA
SEQ ID NO: 1
GAGGAAACTT AAGAUGGG
SEQ ID NO: 2
GGAUGGG
SEQ ID NO: 3
GGAUAGG
SEQ ID NO: 4
GGAGAGG
SEQ ID NO: 58
GGGAUGGG
SEQ ID NO: 59
GAGAGG
SEQ ID NO: 60
GAGGG
SEQ ID NO: 61
GAGAUGGG
SEQ ID NO: 62
GAGUGG
SEQ ID NO: 63
GAGGGG
SEQ ID NO: 64
GAGUAGG
SEQ ID NO: 65
GAGUGGG
(RNA sequence for a construct with two subgenomic promoters, Luc, and E3L)
SEQ ID NO: 127
atgggcggcgcatgagagaagcccagaccaattacctacccaaaatggagaaagttcacgttgacatcgaggaagacagcccatt
cctcagagctttgcagcggagcttcccgcagtttgaggtagaagccaagcaggtcactgataatgaccatgctaatgccagagcgtt
ttcgcatctggcttcaaaactgatcgaaacggaggtggacccatccgacacgatccttgacattggaagtgcgcccgcccgcagaat
gtattctaagcacaagtatcattgtatctgtccgatgagatgtgcggaagatccggacagattgtataagtatgcaactaagctgaa
gaaaaactgtaaggaaataactgataaggaattggacaagaaaatgaaggagctggccgccgtcatgagcgaccctgacctgga
aactgagactatgtgcctccacgacgacgagtcgtgtcgctacgaagggcaagtcgctgtttaccaggatgtatacgcCGTCGAC
GGCCCCACCAGCCTGTACCACCAGGCCAACAAGGGCGTGAGGGTGGCCTACTGGATCGGCTTCGAC
ACCACACCCTTCATGTTCAAGAACCTGGCCGGCGCCTACCCCAGCTACAGCACCAACTGGGCCGACG
AGACCGTGCTGACCGCCAGGAACATCGGCCTGTGCAGCAGCGACGTGATGGAGAGGAGCCGGAGA
GGCATGAGCATCCTGAGGAAGAAATACCTGAAGCCCAGCAACAACGTGCTGTTCAGCGTGGGCAGC
ACCATCTACCACGAGAAGAGGGACCTGCTCAGGAGCTGGCACCTGCCCAGCGTGTTCCACCTGAGG
GGCAAGCAGAACTACACCTGCAGGTGCGAGACCATCGTGAGCTGCGACGGCTACGTGGTGAAGAG
GATCGCCATCAGCCCCGGCCTGTACGGCAAGCCCAGCGGCTACGCCGCTACAATGCACAGGGAGGG
CTTCCTGTGCTGCAAGGTGACCGACACCCTGAACGGCGAGAGGGTGAGCTTCCCCGTGTGCACCTA
CGTGCCCGCCACCCTGTGCGACCAGATGACCGGCATCCTGGCCACCGACGTGAGCGCCGACGACGC
CCAGAAGCTGCTCGTGGGCCTGAACCAGAGGATCGTGGTCAACGGCAGGACCCAGAGGAACACCA
ACACAATGAAGAACTACCTGCTGCCCGTGGTGGCCCAGGCTTTCGCCAGGTGGGCCAAGGAGTACA
AGGAGGACCAGGAAGACGAGAGGCCCCTGGGCCTGAGGGACAGGCAGCTGGTGATGGGCTGCTG
CTGGGCCTTCAGGCGGCACAAGATCACCAGCATCTACAAGAGGCCCGACACCCAGACCATCATCAA
GGTGAACAGCGACTTCCACAGCTTCGTGCTGCCCAGGATCGGCAGCAACACCCTGGAGATCGGCCT
GAGGACCCGGATCAGGAAGATGCTGGAGGAACACAAGGAGCCCAGCCCACTGATCACCGCCGAGG
ACGTGCAGGAGGCCAAGTGCGCTGCCGACGAGGCCAAGGAGGTGAGGGAGGCCGAGGAACTGAG
GGCCGCCCTGCCACCCCTGGCTGCCGACGTGGAGGAACCCACCCTGGAAGCCGACGTGGACCTGAT
GCTGCAGGAGGCCGGCGCCGGAAGCGTGGAGACACCCAGGGGCCTGATCAAGGTGACCAGCTACG
ACGGCGAGGACAAGATCGGCAGCTACGCCGTGCTGAGCCCACAGGCCGTGCTGAAGTCCGAGAAG
CTGAGCTGCATCCACCCACTGGCCGAGCAGGTGATCGTGATCACCCACAGCGGCAGGAAGGGCAG
GTACGCCGTGGAGCCCTACCACGGCAAGGTGGTCGTGCCCGAGGGCCACGCCATCCCCGTGCAGGA
CTTCCAGGCCCTGAGCGAGAGCGCCACCATCGTGTACAACGAGAGGGAGTTCGTGAACAGGTACCT
GCACCATATCGCCACCCACGGCGGAGCCCTGAACACCGACGAGGAATACTACAAGACCGTGAAGCC
CAGCGAGCACGACGGCGAGTACCTGTACGACATCGACAGGAAGCAGTGCGTGAAGAAAGAGCTGG
TGACCGGCCTGGGACTGACCGGCGAGCTGGTGGACCCACCCTTCCACGAGTTCGCCTACGAGAGCC
TGAGGACCAGACCCGCCGCTCCCTACCAGGTGCCCACCATCGGCGTGTACGGCGTGCCCGGCAGCG
GAAAGAGCGGCATCATCAAGAGCGCCGTGACCAAGAAAGACCTGGTGGTCAGCGCCAAGAAAGAG
AACTGCGCCGAGATCATCAGGGACGTGAAGAAGATGAAAGGCCTGGACGTGAACGCGCGCACCGT
GGACAGCGTGCTGCTGAACGGCTGCAAGCACCCCGTGGAGACCCTGTACATCGACGAGGCCTTCGC
TTGCCACGCCGGCACCCTGAGGGCCCTGATCGCCATCATCAGGCCCAAGAAAGCCGTGCTGTGCGG
CGACCCCAAGCAGTGCGGCTTCTTCAACATGATGTGCCTGAAGGTGCACTTCAACCACGAGATCTGC
ACCCAGGTGTTCCACAAGAGCATCAGCAGGCGGTGCACCAAGAGCGTGACCAGCGTCGTGAGCACC
CTGTTCTACGACAAGAAAATGAGGACCACCAACCCCAAGGAGACCAAAATCGTGATCGACACCACA
GGCAGCACCAAGCCCAAGCAGGACGACCTGATCCTGACCTGCTTCAGGGGCTGGGTGAAGCAGCTG
CAGATCGACTACAAGGGCAACGAGATCATGACCGCCGCTGCCAGCCAGGGCCTGACCAGGAAGGG
CGTGTACGCCGTGAGGTACAAGGTGAACGAGAACCCACTGTACGCTCCCACCAGCGAGCACGTGAA
CGTGCTGCTGACCAGGACCGAGGACAGGATCGTGTGGAAGACCCTGGCCGGCGACCCCTGGATCA
AGACCCTGACCGCCAAGTACCCCGGCAACTTCACCGCCACCATCGAAGAGTGGCAGGCCGAGCACG
ACGCCATCATGAGGCACATCCTGGAGAGGCCCGACCCCACCGACGTGTTCCAGAACAAGGCCAACG
TGTGCTGGGCCAAGGCCCTGGTGCCCGTGCTGAAGACCGCCGGCATCGACATGACCACAGAGCAGT
GGAACACCGTGGACTACTTCGAGACCGACAAGGCCCACAGCGCCGAGATCGTGCTGAACCAGCTGT
GCGTGAGGTTCTTCGGCCTGGACCTGGACAGCGGCCTGTTCAGCGCCCCCACCGTGCCACTGAGCAT
CAGGAACAACCACTGGGACAACAGCCCCAGCCCAAACATGTACGGCCTGAACAAGGAGGTGGTCA
GGCAGCTGAGCAGGCGGTACCCACAGCTGCCCAGGGCCGTGGCCACCGGCAGGGTGTACGACATG
AACACCGGCACCCTGAGGAACTACGACCCCAGGATCAACCTGGTGCCCGTGAACAGGCGGCTGCCC
CACGCCCTGGTGCTGCACCACAACGAGCACCCACAGAGCGACTTCAGCTCCTTCGTGAGCAAGCTGA
AAGGCAGGACCGTGCTGGTCGTGGGCGAGAAGCTGAGCGTGCCCGGCAAGATGGTGGACTGGCTG
AGCGACAGGCCCGAGGCCACCTTCCGGGCCAGGCTGGACCTCGGCATCCCCGGCGACGTGCCCAAG
TACGACATCATCTTCGTGAACGTCAGGACCCCATACAAGTACCACCATTACCAGCAGTGCGAGGACC
ACGCCATCAAGCTGAGCATGCTGACCAAGAAGGCCTGCCTGCACCTGAACCCCGGAGGCACCTGCG
TGAGCATCGGCTACGGCTACGCCGACAGGGCCAGCGAGAGCATCATTGGCGCCATCGCCAGGCTGT
TCAAGTTCAGCAGGGTGTGCAAACCCAAGAGCAGCCTGGAGGAAACCGAGGTGCTGTTCGTGTTCA
TCGGCTACGACCGGAAGGCCAGGACCCACAACCCCTACAAGCTGAGCAGCACCCTGACAAACATCT
ACACCGGCAGCAGGCTGCACGAGGCCGGCTGCGCCCCCAGCTACCACGTGGTCAGGGGCGATATC
GCCACCGCCACCGAGGGCGTGATCATCAACGCTGCCAACAGCAAGGGCCAGCCCGGAGGCGGAGT
GTGCGGCGCCCTGTACAAGAAGTTCCCCGAGAGCTTCGACCTGCAGCCCATCGAGGTGGGCAAGGC
CAGGCTGGTGAAGGGCGCCGCTAAGCACATCATCCACGCCGTGGGCCCCAACTTCAACAAGGTGAG
CGAGGTGGAAGGCGACAAGCAGCTGGCCGAAGCCTACGAGAGCATCGCCAAGATCGTGAACGACA
ATAACTACAAGAGCGTGGCCATCCCACTGCTCAGCACCGGCATCTTCAGCGGCAACAAGGACAGGC
TGACCCAGAGCCTGAACCACCTGCTCACCGCCCTGGACACCACCGATGCCGACGTGGCCATCTACTG
CAGGGACAAGAAGTGGGAGATGACCCTGAAGGAGGCCGTGGCCAGGCGGGAGGCCGTGGAAGAG
ATCTGCATCAGCGACGACTCCAGCGTGACCGAGCCCGACGCCGAGCTGGTGAGGGTGCACCCCAAG
AGCTCCCTGGCCGGCAGGAAGGGCTACAGCACCAGCGACGGCAAGACCTTCAGCTACCTGGAGGG
CACCAAGTTCCACCAGGCCGCTAAGGACATCGCCGAGATCAACGCTATGTGGCCCGTGGCCACCGA
GGCCAACGAGCAGGTGTGCATGTACATCCTGGGCGAGAGCATGTCCAGCATCAGGAGCAAGTGCCC
CGTGGAGGAAAGCGAGGCCAGCACACCACCCAGCACCCTGCCCTGCCTGTGCATCCACGCTATGAC
ACCCGAGAGGGTGCAGCGGCTGAAGGCCAGCAGGCCCGAGCAGATCACCGTGTGCAGCTCCTTCCC
ACTGCCCAAGTACAGGATCACCGGCGTGCAGAAGATCCAGTGCAGCCAGCCCATCCTGTTCAGCCCA
AAGGTGCCCGCCTACATCCACCCCAGGAAGTACCTGGTGGAGACCCCACCCGTGGACGAGACACCC
GAGCCAAGCGCCGAGAACCAGAGCACCGAGGGCACACCCGAGCAGCCACCCCTGATCACCGAGGA
CGAGACAAGGACCCGGACCCCAGAGCCCATCATTATCGAGGAAGAGGAAGAGGACAGCATCAGCC
TGCTGAGCGACGGCCCCACCCACCAGGTGCTGCAGGTGGAGGCCGACATCCACGGCCCACCCAGCG
TGTCCAGCTCCAGCTGGAGCATCCCACACGCCAGCGACTTCGACGTGGACAGCCTGAGCATCCTGG
ACACCCTGGAGGGCGCCAGCGTGACCTCCGGCGCCACCAGCGCCGAGACCAACAGCTACTTCGCCA
AGAGCATGGAGTTCCTGGCCAGGCCCGTGCCAGCTCCCAGGACCGTGTTCAGGAACCCACCCCACC
CAGCTCCCAGGACCAGGACCCCAAGCCTGGCTCCCAGCAGGGCCTGCAGCAGGACCAGCCTGGTGA
GCACCCCACCCGGCGTGAACAGGGTGATCACCAGGGAGGAACTGGAGGCCCTGACACCCAGCAGG
ACCCCCAGCAGGTCCGTGAGCAGGACTAGTCTGGTGTCCAACCCACCCGGCGTGAACAGGGTGATC
ACCAGGGAGGAATTCGAGGCCTTCGTGGCCCAGCAACAGAGACGGTTCGACGCCGGCGCCTACATC
TTCAGCAGCGACACCGGCCAGGGACACCTGCAGCAAAAGAGCGTGAGGCAGACCGTGCTGAGCGA
GGTGGTGCTGGAGAGGACCGAGCTGGAAATCAGCTACGCCCCCAGGCTGGACCAGGAGAAGGAG
GAACTGCTCAGGAAGAAACTGCAGCTGAACCCCACCCCAGCCAACAGGAGCAGGTACCAGAGCAG
GAAGGTGGAGAACATGAAGGCCATCACCGCCAGGCGGATCCTGCAGGGCCTGGGACACTACCTGA
AGGCCGAGGGCAAGGTGGAGTGCTACAGGACCCTGCACCCCGTGCCACTGTACAGCTCCAGCGTGA
ACAGGGCCTTCTCCAGCCCCAAGGTGGCCGTGGAGGCCTGCAACGCTATGCTGAAGGAGAACTTCC
CCACCGTGGCCAGCTACTGCATCATCCCCGAGTACGACGCCTACCTGGACATGGTGGACGGCGCCA
GCTGCTGCCTGGACACCGCCAGCTTCTGCCCCGCCAAGCTGAGGAGCTTCCCCAAGAAACACAGCTA
CCTGGAGCCCACCATCAGGAGCGCCGTGCCCAGCGCCATCCAGAACACCCTGCAGAACGTGCTGGC
CGCTGCCACCAAGAGGAACTGCAACGTGACCCAGATGAGGGAGCTGCCCGTGCTGGACAGCGCTG
CCTTCAACGTGGAGTGCTTCAAGAAATACGCCTGCAACAACGAGTACTGGGAGACCTTCAAGGAGA
ACCCCATCAGGCTGACCGAAGAGAACGTGGTGAACTACATCACCAAGCTGAAGGGCCCCAAGGCCG
CTGCCCTGTTCGCTAAGACCCACAACCTGAACATGCTGCAGGACATCCCAATGGACAGGTTCGTGAT
GGACCTGAAGAGGGACGTGAAGGTGACACCCGGCACCAAGCACACCGAGGAGAGGCCCAAGGTG
CAGGTGATCCAGGCCGCTGACCCACTGGCCACCGCCTACCTGTGCGGCATCCACAGGGAGCTGGTG
AGGCGGCTGAACGCCGTGCTGCTGCCCAACATCCACACCCTGTTCGACATGAGCGCCGAGGACTTC
GACGCCATCATCGCCGAGCACTTCCAGCCCGGCGACTGCGTGCTGGAGACCGACATCGCCAGCTTC
GACAAGAGCGAGGATGACGCTATGGCCCTGACCGCTCTGATGATCCTGGAGGACCTGGGCGTGGA
CGCCGAGCTGCTCACCCTGATCGAGGCTGCCTTCGGCGAGATCAGCTCCATCCACCTGCCCACCAAG
ACCAAGTTCAAGTTCGGCGCTATGATGAAAAGCGGAATGTTCCTGACCCTGTTCGTGAACACCGTGA
TCAACATTGTGATCGCCAGCAGGGTGCTGCGGGAGAGGCTGACCGGCAGCCCCTGCGCTGCCTTCA
TCGGCGACGACAACATCGTGAAGGGCGTGAAAAGCGACAAGCTGATGGCCGACAGGTGCGCCACC
TGGCTGAACATGGAGGTGAAGATCATCGACGCCGTGGTGGGCGAGAAGGCCCCCTACTTCTGCGGC
GGATTCATCCTGTGCGACAGCGTGACCGGCACCGCCTGCAGGGTGGCCGACCCCCTGAAGAGGCTG
TTCAAGCTGGGCAAGCCACTGGCCGCTGACGATGAGCACGACGATGACAGGCGGAGGGCCCTGCA
CGAGGAAAGCACCAGGTGGAACAGGGTGGGCATCCTGAGCGAGCTGTGCAAGGCCGTGGAGAGC
AGGTACGAGACCGTGGGCACCAGCATCATCGTGATGGCTATGACCACACTGGCCAGCTCCGTCAAG
AGCTTCTCCTACCTGAGGGGGGCCCCTATAACTCTCTACGGCTAACCTGAATGGACTACGACATAGT
CTAgccaccATGagcaagatctacatcgacgagcggagcaacgccgagatcgtgtgcgaggccatcaagaccatcggcatcga
gggcgccaccgccgcccagctgaccaggcagctgaacatggagaagcgggaggtgaacaaggccctgtacgacctgcagaggag
cgctatggtgtactccagcgacgacatccctccccggtggttcatgaccaccgaggccgacaagcccgacgccgacgctatggccg
acgtgatcatcgacgacgtgagcagggagaagtccatgagggaggaccacaagagcttcgacgacgtgatccccgccaagaaga
tcatcgactggaagggcgccaaccccgtgaccgtgatcaacgagtactgccagatcaccaggagggactggagcttccggatcga
gagcgtgggccccagcaacagccccaccttctacgcctgcgtggacatcgacggcagggtgttcgacaaggccgacggcaagagc
aagcgggacgccaagaacaacgccgccaagctggccgtggacaagctgctgggctacgtgatcatccggttcTAAactcgagcta
gtgactgactaggatctggttaccactaaaccagcctcaagaacacccgaatggagtctctaagctacataataccaacttacactt
acaaaatgttgtcccccaaaatgtagccattcgtatctgctcctaataaaaagaaagtttcttcacattctagAGCTCCGTCAAG
AGCTTCTCCTACCTGAGGGGGGCCCCTATAACTCTCTACGGCTAACCTGAATGGACTACGACATAGT
CTAGCCACCATGGAAGATGCCAAAAACATTAAGAAGGGCCCAGCGCCATTCTACCCACTCGAAGAC
GGGACCGCCGGCGAGCAGCTGCACAAAGCCATGAAGCGCTACGCCCTGGTGCCCGGCACCATCGCC
TTTACCGACGCACATATCGAGGTGGACATTACCTACGCCGAGTACTTCGAGATGAGCGTTCGGCTGG
CAGAAGCTATGAAGCGCTATGGGCTGAATACAAACCATCGGATCGTGGTGTGCAGCGAGAATAGCT
TGCAGTTCTTCATGCCCGTGTTGGGTGCCCTGTTCATCGGTGTGGCTGTGGCCCCAGCTAACGACAT
CTACAACGAGCGCGAGCTGCTGAACAGCATGGGCATCAGCCAGCCCACCGTCGTATTCGTGAGCAA
GAAAGGGCTGCAAAAGATCCTCAACGTGCAAAAGAAGCTACCGATCATACAAAAGATCATCATCAT
GGATAGCAAGACCGACTACCAGGGCTTCCAAAGCATGTACACCTTCGTGACTTCCCATTTGCCACCC
GGCTTCAACGAGTACGACTTCGTGCCCGAGAGCTTCGACCGGGACAAAACCATCGCCCTGATCATGA
ACAGTAGTGGCAGTACCGGATTGCCCAAGGGCGTAGCCCTACCGCACCGCACCGCTTGTGTCCGATT
CAGTCATGCCCGCGACCCCATCTTCGGCAACCAGATCATCCCCGACACCGCTATCCTCAGCGTGGTG
CCATTTCACCACGGCTTCGGCATGTTCACCACGCTGGGCTACTTGATCTGCGGCTTTCGGGTCGTGCT
CATGTACCGCTTCGAGGAGGAGCTATTCTTGCGCAGCTTGCAAGACTATAAGATTCAATCTGCCCTG
CTGGTGCCCACACTATTTAGCTTCTTCGCTAAGAGCACTCTCATCGACAAGTACGACCTAAGCAACTT
GCACGAGATCGCCAGCGGCGGGGCGCCGCTCAGCAAGGAGGTAGGTGAGGCCGTGGCCAAACGC
TTCCACCTACCAGGCATCCGACAGGGCTACGGCCTGACAGAAACAACCAGCGCCATTCTGATCACCC
CCGAAGGGGACGACAAGCCTGGCGCAGTAGGCAAGGTGGTGCCCTTCTTCGAGGCTAAGGTGGTG
GACTTGGACACCGGTAAGACACTGGGTGTGAACCAGCGCGGCGAGCTGTGCGTCCGTGGCCCCATG
ATCATGAGCGGCTACGTTAACAACCCCGAGGCTACAAACGCTCTCATCGACAAGGACGGCTGGCTG
CACAGCGGCGACATCGCCTACTGGGACGAGGACGAGCACTTCTTCATCGTGGACCGGCTGAAGTCC
CTGATCAAATACAAGGGCTACCAGGTAGCCCCAGCCGAACTGGAGAGCATCCTGCTGCAACACCCC
AACATCTTCGACGCCGGGGTCGCCGGCCTGCCCGACGACGATGCCGGCGAGCTGCCCGCCGCAGTC
GTCGTGCTGGAACACGGTAAAACCATGACCGAGAAGGAGATCGTGGACTATGTGGCCAGCCAGGTT
ACAACCGCCAAGAAGCTGCGCGGTGGTGTTGTGTTCGTGGACGAGGTGCCTAAAGGACTGACCGG
CAAGTTGGACGCCCGCAAGATCCGCGAGATTCTCATTAAGGCCAAGAAGGGCGGCAAGATCGCCGT
GTAACTCGAGTATGTTACGTGCAAAGGTGATTGTCACCCCCCGAAAGACCATATTGTGACACACCCT
CAGTATCACGCCCAAACATTTACAGCCGCGGTGTCAAAAACCGCGTGGACGTGGTTAACATCCCTGC
TGGGAGGATCAGCCGTAATTATTATAATTGGCTTGGTGCTGGCTACTATTGTGGCCATGTACGTGCT
GACCAACCAGAAACATAATTGAATACAGCAGCAATTGGCAAGCTGCTTACATAGAACTCGCGGCGA
TTGGCATGCCGCCTTAAAATTTTTATTTTATTTTTTCTTTTCTTTTCCGAATCGGATTTTGTTTTTAATAT
TTCAAAAAAAAAAAAAAAAAAAAAAAAATctagAAAAAAAAAAAAAAAAAAAAAAAAAAAAAAAAA
AAAAAAAAAAAAAAAAAAAAAAAAAAAAAAAAAAAAAAAAAAAAAAAaaaaaaaaaaaaaaaaaaaa
(RNA sequence for STARR Fluc IRES-E3L)
SEQ ID NO: 128
AUGGGCGGCGCAUGAGAGAAGCCCAGACCAAUUACCUACCCAAAAUGGAGAAA
GUUCACGUUGACAUCGAGGAAGACAGCCCAUUCCUCAGAGCUUUGCAGCGGAG
CUUCCCGCAGUUUGAGGUAGAAGCCAAGCAGGUCACUGAUAAUGACCAUGCUA
AUGCCAGAGCGUUUUCGCAUCUGGCUUCAAAACUGAUCGAAACGGAGGUGGA
CCCAUCCGACACGAUCCUUGACAUUGGAAGUGCGCCCGCCCGCAGAAUGUAUU
CUAAGCACAAGUAUCAUUGUAUCUGUCCGAUGAGAUGUGCGGAAGAUCCGGA
CAGAUUGUAUAAGUAUGCAACUAAGCUGAAGAAAAACUGUAAGGAAAUAACU
GAUAAGGAAUUGGACAAGAAAAUGAAGGAGCUGGCCGCCGUCAUGAGCGACC
CUGACCUGGAAACUGAGACUAUGUGCCUCCACGACGACGAGUCGUGUCGCUAC
GAAGGGCAAGUCGCUGUUUACCAGGAUGUAUACGCCGUCGACGGCCCCACCAG
CCUGUACCACCAGGCCAACAAGGGCGUGAGGGUGGCCUACUGGAUCGGCUUCG
ACACCACACCCUUCAUGUUCAAGAACCUGGCCGGCGCCUACCCCAGCUACAGC
ACCAACUGGGCCGACGAGACCGUGCUGACCGCCAGGAACAUCGGCCUGUGCAG
CAGCGACGUGAUGGAGAGGAGCCGGAGAGGCAUGAGCAUCCUGAGGAAGAAA
UACCUGAAGCCCAGCAACAACGUGCUGUUCAGCGUGGGCAGCACCAUCUACCA
CGAGAAGAGGGACCUGCUCAGGAGCUGGCACCUGCCCAGCGUGUUCCACCUGA
GGGGCAAGCAGAACUACACCUGCAGGUGCGAGACCAUCGUGAGCUGCGACGGC
UACGUGGUGAAGAGGAUCGCCAUCAGCCCCGGCCUGUACGGCAAGCCCAGCGG
CUACGCCGCUACAAUGCACAGGGAGGGCUUCCUGUGCUGCAAGGUGACCGACA
CCCUGAACGGCGAGAGGGUGAGCUUCCCCGUGUGCACCUACGUGCCCGCCACC
CUGUGCGACCAGAUGACCGGCAUCCUGGCCACCGACGUGAGCGCCGACGACGC
CCAGAAGCUGCUCGUGGGCCUGAACCAGAGGAUCGUGGUCAACGGCAGGACCC
AGAGGAACACCAACACAAUGAAGAACUACCUGCUGCCCGUGGUGGCCCAGGCU
UUCGCCAGGUGGGCCAAGGAGUACAAGGAGGACCAGGAAGACGAGAGGCCCCU
GGGCCUGAGGGACAGGCAGCUGGUGAUGGGCUGCUGCUGGGCCUUCAGGCGGC
ACAAGAUCACCAGCAUCUACAAGAGGCCCGACACCCAGACCAUCAUCAAGGUG
AACAGCGACUUCCACAGCUUCGUGCUGCCCAGGAUCGGCAGCAACACCCUGGA
GAUCGGCCUGAGGACCCGGAUCAGGAAGAUGCUGGAGGAACACAAGGAGCCCA
GCCCACUGAUCACCGCCGAGGACGUGCAGGAGGCCAAGUGCGCUGCCGACGAG
GCCAAGGAGGUGAGGGAGGCCGAGGAACUGAGGGCCGCCCUGCCACCCCUGGC
UGCCGACGUGGAGGAACCCACCCUGGAAGCCGACGUGGACCUGAUGCUGCAGG
AGGCCGGCGCCGGAAGCGUGGAGACACCCAGGGGCCUGAUCAAGGUGACCAGC
UACGACGGCGAGGACAAGAUCGGCAGCUACGCCGUGCUGAGCCCACAGGCCGU
GCUGAAGUCCGAGAAGCUGAGCUGCAUCCACCCACUGGCCGAGCAGGUGAUCG
UGAUCACCCACAGCGGCAGGAAGGGCAGGUACGCCGUGGAGCCCUACCACGGC
AAGGUGGUCGUGCCCGAGGGCCACGCCAUCCCCGUGCAGGACUUCCAGGCCCU
GAGCGAGAGCGCCACCAUCGUGUACAACGAGAGGGAGUUCGUGAACAGGUACC
UGCACCAUAUCGCCACCCACGGCGGAGCCCUGAACACCGACGAGGAAUACUAC
AAGACCGUGAAGCCCAGCGAGCACGACGGCGAGUACCUGUACGACAUCGACAG
GAAGCAGUGCGUGAAGAAAGAGCUGGUGACCGGCCUGGGACUGACCGGCGAG
CUGGUGGACCCACCCUUCCACGAGUUCGCCUACGAGAGCCUGAGGACCAGACC
CGCCGCUCCCUACCAGGUGCCCACCAUCGGCGUGUACGGCGUGCCCGGCAGCG
GAAAGAGCGGCAUCAUCAAGAGCGCCGUGACCAAGAAAGACCUGGUGGUCAGC
GCCAAGAAAGAGAACUGCGCCGAGAUCAUCAGGGACGUGAAGAAGAUGAAAG
GCCUGGACGUGAACGCGCGCACCGUGGACAGCGUGCUGCUGAACGGCUGCAAG
CACCCCGUGGAGACCCUGUACAUCGACGAGGCCUUCGCUUGCCACGCCGGCAC
CCUGAGGGCCCUGAUCGCCAUCAUCAGGCCCAAGAAAGCCGUGCUGUGCGGCG
ACCCCAAGCAGUGCGGCUUCUUCAACAUGAUGUGCCUGAAGGUGCACUUCAAC
CACGAGAUCUGCACCCAGGUGUUCCACAAGAGCAUCAGCAGGCGGUGCACCAA
GAGCGUGACCAGCGUCGUGAGCACCCUGUUCUACGACAAGAAAAUGAGGACCA
CCAACCCCAAGGAGACCAAAAUCGUGAUCGACACCACAGGCAGCACCAAGCCC
AAGCAGGACGACCUGAUCCUGACCUGCUUCAGGGGCUGGGUGAAGCAGCUGCA
GAUCGACUACAAGGGCAACGAGAUCAUGACCGCCGCUGCCAGCCAGGGCCUGA
CCAGGAAGGGCGUGUACGCCGUGAGGUACAAGGUGAACGAGAACCCACUGUAC
GCUCCCACCAGCGAGCACGUGAACGUGCUGCUGACCAGGACCGAGGACAGGAU
CGUGUGGAAGACCCUGGCCGGCGACCCCUGGAUCAAGACCCUGACCGCCAAGU
ACCCCGGCAACUUCACCGCCACCAUCGAAGAGUGGCAGGCCGAGCACGACGCC
AUCAUGAGGCACAUCCUGGAGAGGCCCGACCCCACCGACGUGUUCCAGAACAA
GGCCAACGUGUGCUGGGCCAAGGCCCUGGUGCCCGUGCUGAAGACCGCCGGCA
UCGACAUGACCACAGAGCAGUGGAACACCGUGGACUACUUCGAGACCGACAAG
GCCCACAGCGCCGAGAUCGUGCUGAACCAGCUGUGCGUGAGGUUCUUCGGCCU
GGACCUGGACAGCGGCCUGUUCAGCGCCCCCACCGUGCCACUGAGCAUCAGGA
ACAACCACUGGGACAACAGCCCCAGCCCAAACAUGUACGGCCUGAACAAGGAG
GUGGUCAGGCAGCUGAGCAGGCGGUACCCACAGCUGCCCAGGGCCGUGGCCAC
CGGCAGGGUGUACGACAUGAACACCGGCACCCUGAGGAACUACGACCCCAGGA
UCAACCUGGUGCCCGUGAACAGGCGGCUGCCCCACGCCCUGGUGCUGCACCAC
AACGAGCACCCACAGAGCGACUUCAGCUCCUUCGUGAGCAAGCUGAAAGGCAG
GACCGUGCUGGUCGUGGGCGAGAAGCUGAGCGUGCCCGGCAAGAUGGUGGAC
UGGCUGAGCGACAGGCCCGAGGCCACCUUCCGGGCCAGGCUGGACCUCGGCAU
CCCCGGCGACGUGCCCAAGUACGACAUCAUCUUCGUGAACGUCAGGACCCCAU
ACAAGUACCACCAUUACCAGCAGUGCGAGGACCACGCCAUCAAGCUGAGCAUG
CUGACCAAGAAGGCCUGCCUGCACCUGAACCCCGGAGGCACCUGCGUGAGCAU
CGGCUACGGCUACGCCGACAGGGCCAGCGAGAGCAUCAUUGGCGCCAUCGCCA
GGCUGUUCAAGUUCAGCAGGGUGUGCAAACCCAAGAGCAGCCUGGAGGAAACC
GAGGUGCUGUUCGUGUUCAUCGGCUACGACCGGAAGGCCAGGACCCACAACCC
CUACAAGCUGAGCAGCACCCUGACAAACAUCUACACCGGCAGCAGGCUGCACG
AGGCCGGCUGCGCCCCCAGCUACCACGUGGUCAGGGGCGAUAUCGCCACCGCC
ACCGAGGGCGUGAUCAUCAACGCUGCCAACAGCAAGGGCCAGCCCGGAGGCGG
AGUGUGCGGCGCCCUGUACAAGAAGUUCCCCGAGAGCUUCGACCUGCAGCCCA
UCGAGGUGGGCAAGGCCAGGCUGGUGAAGGGCGCCGCUAAGCACAUCAUCCAC
GCCGUGGGCCCCAACUUCAACAAGGUGAGCGAGGUGGAAGGCGACAAGCAGCU
GGCCGAAGCCUACGAGAGCAUCGCCAAGAUCGUGAACGACAAUAACUACAAGA
GCGUGGCCAUCCCACUGCUCAGCACCGGCAUCUUCAGCGGCAACAAGGACAGG
CUGACCCAGAGCCUGAACCACCUGCUCACCGCCCUGGACACCACCGAUGCCGA
CGUGGCCAUCUACUGCAGGGACAAGAAGUGGGAGAUGACCCUGAAGGAGGCC
GUGGCCAGGCGGGAGGCCGUGGAAGAGAUCUGCAUCAGCGACGACUCCAGCGU
GACCGAGCCCGACGCCGAGCUGGUGAGGGUGCACCCCAAGAGCUCCCUGGCCG
GCAGGAAGGGCUACAGCACCAGCGACGGCAAGACCUUCAGCUACCUGGAGGGC
ACCAAGUUCCACCAGGCCGCUAAGGACAUCGCCGAGAUCAACGCUAUGUGGCC
CGUGGCCACCGAGGCCAACGAGCAGGUGUGCAUGUACAUCCUGGGCGAGAGCA
UGUCCAGCAUCAGGAGCAAGUGCCCCGUGGAGGAAAGCGAGGCCAGCACACCA
CCCAGCACCCUGCCCUGCCUGUGCAUCCACGCUAUGACACCCGAGAGGGUGCA
GCGGCUGAAGGCCAGCAGGCCCGAGCAGAUCACCGUGUGCAGCUCCUUCCCAC
UGCCCAAGUACAGGAUCACCGGCGUGCAGAAGAUCCAGUGCAGCCAGCCCAUC
CUGUUCAGCCCAAAGGUGCCCGCCUACAUCCACCCCAGGAAGUACCUGGUGGA
GACCCCACCCGUGGACGAGACACCCGAGCCAAGCGCCGAGAACCAGAGCACCG
AGGGCACACCCGAGCAGCCACCCCUGAUCACCGAGGACGAGACAAGGACCCGG
ACCCCAGAGCCCAUCAUUAUCGAGGAAGAGGAAGAGGACAGCAUCAGCCUGCU
GAGCGACGGCCCCACCCACCAGGUGCUGCAGGUGGAGGCCGACAUCCACGGCC
CACCCAGCGUGUCCAGCUCCAGCUGGAGCAUCCCACACGCCAGCGACUUCGAC
GUGGACAGCCUGAGCAUCCUGGACACCCUGGAGGGCGCCAGCGUGACCUCCGG
CGCCACCAGCGCCGAGACCAACAGCUACUUCGCCAAGAGCAUGGAGUUCCUGG
CCAGGCCCGUGCCAGCUCCCAGGACCGUGUUCAGGAACCCACCCCACCCAGCUC
CCAGGACCAGGACCCCAAGCCUGGCUCCCAGCAGGGCCUGCAGCAGGACCAGC
CUGGUGAGCACCCCACCCGGCGUGAACAGGGUGAUCACCAGGGAGGAACUGGA
GGCCCUGACACCCAGCAGGACCCCCAGCAGGUCCGUGAGCAGGACUAGUCUGG
UGUCCAACCCACCCGGCGUGAACAGGGUGAUCACCAGGGAGGAAUUCGAGGCC
UUCGUGGCCCAGCAACAGAGACGGUUCGACGCCGGCGCCUACAUCUUCAGCAG
CGACACCGGCCAGGGACACCUGCAGCAAAAGAGCGUGAGGCAGACCGUGCUGA
GCGAGGUGGUGCUGGAGAGGACCGAGCUGGAAAUCAGCUACGCCCCCAGGCUG
GACCAGGAGAAGGAGGAACUGCUCAGGAAGAAACUGCAGCUGAACCCCACCCC
AGCCAACAGGAGCAGGUACCAGAGCAGGAAGGUGGAGAACAUGAAGGCCAUC
ACCGCCAGGCGGAUCCUGCAGGGCCUGGGACACUACCUGAAGGCCGAGGGCAA
GGUGGAGUGCUACAGGACCCUGCACCCCGUGCCACUGUACAGCUCCAGCGUGA
ACAGGGCCUUCUCCAGCCCCAAGGUGGCCGUGGAGGCCUGCAACGCUAUGCUG
AAGGAGAACUUCCCCACCGUGGCCAGCUACUGCAUCAUCCCCGAGUACGACGC
CUACCUGGACAUGGUGGACGGCGCCAGCUGCUGCCUGGACACCGCCAGCUUCU
GCCCCGCCAAGCUGAGGAGCUUCCCCAAGAAACACAGCUACCUGGAGCCCACC
AUCAGGAGCGCCGUGCCCAGCGCCAUCCAGAACACCCUGCAGAACGUGCUGGC
CGCUGCCACCAAGAGGAACUGCAACGUGACCCAGAUGAGGGAGCUGCCCGUGC
UGGACAGCGCUGCCUUCAACGUGGAGUGCUUCAAGAAAUACGCCUGCAACAAC
GAGUACUGGGAGACCUUCAAGGAGAACCCCAUCAGGCUGACCGAAGAGAACGU
GGUGAACUACAUCACCAAGCUGAAGGGCCCCAAGGCCGCUGCCCUGUUCGCUA
AGACCCACAACCUGAACAUGCUGCAGGACAUCCCAAUGGACAGGUUCGUGAUG
GACCUGAAGAGGGACGUGAAGGUGACACCCGGCACCAAGCACACCGAGGAGAG
GCCCAAGGUGCAGGUGAUCCAGGCCGCUGACCCACUGGCCACCGCCUACCUGU
GCGGCAUCCACAGGGAGCUGGUGAGGCGGCUGAACGCCGUGCUGCUGCCCAAC
AUCCACACCCUGUUCGACAUGAGCGCCGAGGACUUCGACGCCAUCAUCGCCGA
GCACUUCCAGCCCGGCGACUGCGUGCUGGAGACCGACAUCGCCAGCUUCGACA
AGAGCGAGGAUGACGCUAUGGCCCUGACCGCUCUGAUGAUCCUGGAGGACCUG
GGCGUGGACGCCGAGCUGCUCACCCUGAUCGAGGCUGCCUUCGGCGAGAUCAG
CUCCAUCCACCUGCCCACCAAGACCAAGUUCAAGUUCGGCGCUAUGAUGAAAA
GCGGAAUGUUCCUGACCCUGUUCGUGAACACCGUGAUCAACAUUGUGAUCGCC
AGCAGGGUGCUGCGGGAGAGGCUGACCGGCAGCCCCUGCGCUGCCUUCAUCGG
CGACGACAACAUCGUGAAGGGCGUGAAAAGCGACAAGCUGAUGGCCGACAGG
UGCGCCACCUGGCUGAACAUGGAGGUGAAGAUCAUCGACGCCGUGGUGGGCGA
GAAGGCCCCCUACUUCUGCGGCGGAUUCAUCCUGUGCGACAGCGUGACCGGCA
CCGCCUGCAGGGUGGCCGACCCCCUGAAGAGGCUGUUCAAGCUGGGCAAGCCA
CUGGCCGCUGACGAUGAGCACGACGAUGACAGGCGGAGGGCCCUGCACGAGGA
AAGCACCAGGUGGAACAGGGUGGGCAUCCUGAGCGAGCUGUGCAAGGCCGUG
GAGAGCAGGUACGAGACCGUGGGCACCAGCAUCAUCGUGAUGGCUAUGACCAC
ACUGGCCAGCUCCGUCAAGAGCUUCUCCUACCUGAGGGGGGCCCCUAUAACUC
UCUACGGCUAACCUGAAUGGACUACGACAUAGUCUAGUCCGCCAAGGCCGCCA
CCAUGGAAGAUGCCAAAAACAUUAAGAAGGGCCCAGCGCCAUUCUACCCACUC
GAAGACGGGACCGCCGGCGAGCAGCUGCACAAAGCCAUGAAGCGCUACGCCCU
GGUGCCCGGCACCAUCGCCUUUACCGACGCACAUAUCGAGGUGGACAUUACCU
ACGCCGAGUACUUCGAGAUGAGCGUUCGGCUGGCAGAAGCUAUGAAGCGCUA
UGGGCUGAAUACAAACCAUCGGAUCGUGGUGUGCAGCGAGAAUAGCUUGCAG
UUCUUCAUGCCCGUGUUGGGUGCCCUGUUCAUCGGUGUGGCUGUGGCCCCAGC
UAACGACAUCUACAACGAGCGCGAGCUGCUGAACAGCAUGGGCAUCAGCCAGC
CCACCGUCGUAUUCGUGAGCAAGAAAGGGCUGCAAAAGAUCCUCAACGUGCAA
AAGAAGCUACCGAUCAUACAAAAGAUCAUCAUCAUGGAUAGCAAGACCGACU
ACCAGGGCUUCCAAAGCAUGUACACCUUCGUGACUUCCCAUUUGCCACCCGGC
UUCAACGAGUACGACUUCGUGCCCGAGAGCUUCGACCGGGACAAAACCAUCGC
CCUGAUCAUGAACAGUAGUGGCAGUACCGGAUUGCCCAAGGGCGUAGCCCUAC
CGCACCGCACCGCUUGUGUCCGAUUCAGUCAUGCCCGCGACCCCAUCUUCGGC
AACCAGAUCAUCCCCGACACCGCUAUCCUCAGCGUGGUGCCAUUUCACCACGG
CUUCGGCAUGUUCACCACGCUGGGCUACUUGAUCUGCGGCUUUCGGGUCGUGC
UCAUGUACCGCUUCGAGGAGGAGCUAUUCUUGCGCAGCUUGCAAGACUAUAA
GAUUCAAUCUGCCCUGCUGGUGCCCACACUAUUUAGCUUCUUCGCUAAGAGCA
CUCUCAUCGACAAGUACGACCUAAGCAACUUGCACGAGAUCGCCAGCGGCGGG
GCGCCGCUCAGCAAGGAGGUAGGUGAGGCCGUGGCCAAACGCUUCCACCUACC
AGGCAUCCGACAGGGCUACGGCCUGACAGAAACAACCAGCGCCAUUCUGAUCA
CCCCCGAAGGGGACGACAAGCCUGGCGCAGUAGGCAAGGUGGUGCCCUUCUUC
GAGGCUAAGGUGGUGGACUUGGACACCGGUAAGACACUGGGUGUGAACCAGC
GCGGCGAGCUGUGCGUCCGUGGCCCCAUGAUCAUGAGCGGCUACGUUAACAAC
CCCGAGGCUACAAACGCUCUCAUCGACAAGGACGGCUGGCUGCACAGCGGCGA
CAUCGCCUACUGGGACGAGGACGAGCACUUCUUCAUCGUGGACCGGCUGAAGU
CCCUGAUCAAAUACAAGGGCUACCAGGUAGCCCCAGCCGAACUGGAGAGCAUC
CUGCUGCAACACCCCAACAUCUUCGACGCCGGGGUCGCCGGCCUGCCCGACGA
CGAUGCCGGCGAGCUGCCCGCCGCAGUCGUCGUGCUGGAACACGGUAAAACCA
UGACCGAGAAGGAGAUCGUGGACUAUGUGGCCAGCCAGGUUACAACCGCCAAG
AAGCUGCGCGGUGGUGUUGUGUUCGUGGACGAGGUGCCUAAAGGACUGACCG
GCAAGUUGGACGCCCGCAAGAUCCGCGAGAUUCUCAUUAAGGCCAAGAAGGGC
GGCAAGAUCGCCGUGUAACUCGAGCCGGAAACGCAAUAGCCGAAAAACAAAAA
ACAAAAAAAACAAAAAAAAAACCAAAAAAACAAAACACAUUAAAACAGCCUG
UGGGUUGAUCCCACCCACAGGCCCAUUGGGCGCUAGCACUCUGGUAUCACGGU
ACCUUUGUGCGCCUGUUUUAUACCCCCUCCCCCAACUGUAACUUAGAAGUAAC
ACACACCGAUCAACAGUCAGCGUGGCACACCAGCCACGUUUUGAUCAAGCACU
UCUGUUACCCCGGACUGAGUAUCAAUAGACUGCUCACGCGGUUGAAGGAGAA
AGCGUUCGUUAUCCGGCCAACUACUUCGAAAAACCUAGUAACACCGUGGAAGU
UGCAGAGUGUUUCGCUCAGCACUACCCCAGUGUAGAUCAGGUCGAUGAGUCAC
CGCAUUCCCCACGGGCGACCGUGGCGGUGGCUGCGUUGGCGGCCUGCCCAUGG
GGAAACCCAUGGGACGCUCUAAUACAGACAUGGUGCGAAGAGUCUAUUGAGC
UAGUUGGUAGUCCUCCGGCCCCUGAAUGCGGCUAAUCCUAACUGCGGAGCACA
CACCCUCAAGCCAGAGGGCAGUGUGUCGUAACGGGCAACUCUGCAGCGGAACC
GACUACUUUGGGUGUCCGUGUUUCAUUUUAUUCCUAUACUGGCUGCUUAUGG
UGACAAUUGAGAGAUCGUUACCAUAUAGCUAUUGGAUUGGCCAUCCGGUGAC
UAAUAGAGCUAUUAUAUAUCCCUUUGUUGGGUUUAUACCACUUAGCUUGAAA
GAGGUUAAAACAUUACAAUUCAUUGUUAAGUUGAAUACAGCAAAAUGAGCAA
GAUCUACAUCGACGAGCGGAGCAACGCCGAGAUCGUGUGCGAGGCCAUCAAGA
CCAUCGGCAUCGAGGGCGCCACCGCCGCCCAGCUGACCAGGCAGCUGAACAUG
GAGAAGCGGGAGGUGAACAAGGCCCUGUACGACCUGCAGAGGAGCGCUAUGG
UGUACUCCAGCGACGACAUCCCUCCCCGGUGGUUCAUGACCACCGAGGCCGAC
AAGCCCGACGCCGACGCUAUGGCCGACGUGAUCAUCGACGACGUGAGCAGGGA
GAAGUCCAUGAGGGAGGACCACAAGAGCUUCGACGACGUGAUCCCCGCCAAGA
AGAUCAUCGACUGGAAGGGCGCCAACCCCGUGACCGUGAUCAACGAGUACUGC
CAGAUCACCAGGAGGGACUGGAGCUUCCGGAUCGAGAGCGUGGGCCCCAGCAA
CAGCCCCACCUUCUACGCCUGCGUGGACAUCGACGGCAGGGUGUUCGACAAGG
CCGACGGCAAGAGCAAGCGGGACGCCAAGAACAACGCCGCCAAGCUGGCCGUG
GACAAGCUGCUGGGCUACGUGAUCAUCCGGUUCUAAACGUAUGUUACGUGCA
AAGGUGAUUGUCACCCCCCGAAAGACCAUAUUGUGACACACCCUCAGUAUCAC
GCCCAAACAUUUACAGCCGCGGUGUCAAAAACCGCGUGGACGUGGUUAACAUC
CCUGCUGGGAGGAUCAGCCGUAAUUAUUAUAAUUGGCUUGGUGCUGGCUACU
AUUGUGGCCAUGUACGUGCUGACCAACCAGAAACAUAAUUGAAUACAGCAGC
AAUUGGCAAGCUGCUUACAUAGAACUCGCGGCGAUUGGCAUGCCGCCUUAAAA
UUUUUAUUUUAUUUUUUCUUUUCUUUUCCGAAUCGGAUUUUGUUUUUAAUAU
UUCAAAAAAAAAAAAAAAAAAAAAAAAAUCUAGAAAAAAAAAAAAAAAAAAA
AAAAAAAAAAAAAAAAAAAAAAAAAAAAAAAAAAAAAAAAAAAAAAAAAAAA
AAAAAAAAAAAAAAAAAAAAAAAAAAAAA
(RNA sequence for STARR Fluc IRES-E3L (short 3′ UTR))
SEQ ID NO: 129
AUGGGCGGCGCAUGAGAGAAGCCCAGACCAAUUACCUACCCAAAAUGGAGAAA
GUUCACGUUGACAUCGAGGAAGACAGCCCAUUCCUCAGAGCUUUGCAGCGGAG
CUUCCCGCAGUUUGAGGUAGAAGCCAAGCAGGUCACUGAUAAUGACCAUGCUA
AUGCCAGAGCGUUUUCGCAUCUGGCUUCAAAACUGAUCGAAACGGAGGUGGA
CCCAUCCGACACGAUCCUUGACAUUGGAAGUGCGCCCGCCCGCAGAAUGUAUU
CUAAGCACAAGUAUCAUUGUAUCUGUCCGAUGAGAUGUGCGGAAGAUCCGGA
CAGAUUGUAUAAGUAUGCAACUAAGCUGAAGAAAAACUGUAAGGAAAUAACU
GAUAAGGAAUUGGACAAGAAAAUGAAGGAGCUGGCCGCCGUCAUGAGCGACC
CUGACCUGGAAACUGAGACUAUGUGCCUCCACGACGACGAGUCGUGUCGCUAC
GAAGGGCAAGUCGCUGUUUACCAGGAUGUAUACGCCGUCGACGGCCCCACCAG
CCUGUACCACCAGGCCAACAAGGGCGUGAGGGUGGCCUACUGGAUCGGCUUCG
ACACCACACCCUUCAUGUUCAAGAACCUGGCCGGCGCCUACCCCAGCUACAGC
ACCAACUGGGCCGACGAGACCGUGCUGACCGCCAGGAACAUCGGCCUGUGCAG
CAGCGACGUGAUGGAGAGGAGCCGGAGAGGCAUGAGCAUCCUGAGGAAGAAA
UACCUGAAGCCCAGCAACAACGUGCUGUUCAGCGUGGGCAGCACCAUCUACCA
CGAGAAGAGGGACCUGCUCAGGAGCUGGCACCUGCCCAGCGUGUUCCACCUGA
GGGGCAAGCAGAACUACACCUGCAGGUGCGAGACCAUCGUGAGCUGCGACGGC
UACGUGGUGAAGAGGAUCGCCAUCAGCCCCGGCCUGUACGGCAAGCCCAGCGG
CUACGCCGCUACAAUGCACAGGGAGGGCUUCCUGUGCUGCAAGGUGACCGACA
CCCUGAACGGCGAGAGGGUGAGCUUCCCCGUGUGCACCUACGUGCCCGCCACC
CUGUGCGACCAGAUGACCGGCAUCCUGGCCACCGACGUGAGCGCCGACGACGC
CCAGAAGCUGCUCGUGGGCCUGAACCAGAGGAUCGUGGUCAACGGCAGGACCC
AGAGGAACACCAACACAAUGAAGAACUACCUGCUGCCCGUGGUGGCCCAGGCU
UUCGCCAGGUGGGCCAAGGAGUACAAGGAGGACCAGGAAGACGAGAGGCCCCU
GGGCCUGAGGGACAGGCAGCUGGUGAUGGGCUGCUGCUGGGCCUUCAGGCGGC
ACAAGAUCACCAGCAUCUACAAGAGGCCCGACACCCAGACCAUCAUCAAGGUG
AACAGCGACUUCCACAGCUUCGUGCUGCCCAGGAUCGGCAGCAACACCCUGGA
GAUCGGCCUGAGGACCCGGAUCAGGAAGAUGCUGGAGGAACACAAGGAGCCCA
GCCCACUGAUCACCGCCGAGGACGUGCAGGAGGCCAAGUGCGCUGCCGACGAG
GCCAAGGAGGUGAGGGAGGCCGAGGAACUGAGGGCCGCCCUGCCACCCCUGGC
UGCCGACGUGGAGGAACCCACCCUGGAAGCCGACGUGGACCUGAUGCUGCAGG
AGGCCGGCGCCGGAAGCGUGGAGACACCCAGGGGCCUGAUCAAGGUGACCAGC
UACGACGGCGAGGACAAGAUCGGCAGCUACGCCGUGCUGAGCCCACAGGCCGU
GCUGAAGUCCGAGAAGCUGAGCUGCAUCCACCCACUGGCCGAGCAGGUGAUCG
UGAUCACCCACAGCGGCAGGAAGGGCAGGUACGCCGUGGAGCCCUACCACGGC
AAGGUGGUCGUGCCCGAGGGCCACGCCAUCCCCGUGCAGGACUUCCAGGCCCU
GAGCGAGAGCGCCACCAUCGUGUACAACGAGAGGGAGUUCGUGAACAGGUACC
UGCACCAUAUCGCCACCCACGGCGGAGCCCUGAACACCGACGAGGAAUACUAC
AAGACCGUGAAGCCCAGCGAGCACGACGGCGAGUACCUGUACGACAUCGACAG
GAAGCAGUGCGUGAAGAAAGAGCUGGUGACCGGCCUGGGACUGACCGGCGAG
CUGGUGGACCCACCCUUCCACGAGUUCGCCUACGAGAGCCUGAGGACCAGACC
CGCCGCUCCCUACCAGGUGCCCACCAUCGGCGUGUACGGCGUGCCCGGCAGCG
GAAAGAGCGGCAUCAUCAAGAGCGCCGUGACCAAGAAAGACCUGGUGGUCAGC
GCCAAGAAAGAGAACUGCGCCGAGAUCAUCAGGGACGUGAAGAAGAUGAAAG
GCCUGGACGUGAACGCGCGCACCGUGGACAGCGUGCUGCUGAACGGCUGCAAG
CACCCCGUGGAGACCCUGUACAUCGACGAGGCCUUCGCUUGCCACGCCGGCAC
CCUGAGGGCCCUGAUCGCCAUCAUCAGGCCCAAGAAAGCCGUGCUGUGCGGCG
ACCCCAAGCAGUGCGGCUUCUUCAACAUGAUGUGCCUGAAGGUGCACUUCAAC
CACGAGAUCUGCACCCAGGUGUUCCACAAGAGCAUCAGCAGGCGGUGCACCAA
GAGCGUGACCAGCGUCGUGAGCACCCUGUUCUACGACAAGAAAAUGAGGACCA
CCAACCCCAAGGAGACCAAAAUCGUGAUCGACACCACAGGCAGCACCAAGCCC
AAGCAGGACGACCUGAUCCUGACCUGCUUCAGGGGCUGGGUGAAGCAGCUGCA
GAUCGACUACAAGGGCAACGAGAUCAUGACCGCCGCUGCCAGCCAGGGCCUGA
CCAGGAAGGGCGUGUACGCCGUGAGGUACAAGGUGAACGAGAACCCACUGUAC
GCUCCCACCAGCGAGCACGUGAACGUGCUGCUGACCAGGACCGAGGACAGGAU
CGUGUGGAAGACCCUGGCCGGCGACCCCUGGAUCAAGACCCUGACCGCCAAGU
ACCCCGGCAACUUCACCGCCACCAUCGAAGAGUGGCAGGCCGAGCACGACGCC
AUCAUGAGGCACAUCCUGGAGAGGCCCGACCCCACCGACGUGUUCCAGAACAA
GGCCAACGUGUGCUGGGCCAAGGCCCUGGUGCCCGUGCUGAAGACCGCCGGCA
UCGACAUGACCACAGAGCAGUGGAACACCGUGGACUACUUCGAGACCGACAAG
GCCCACAGCGCCGAGAUCGUGCUGAACCAGCUGUGCGUGAGGUUCUUCGGCCU
GGACCUGGACAGCGGCCUGUUCAGCGCCCCCACCGUGCCACUGAGCAUCAGGA
ACAACCACUGGGACAACAGCCCCAGCCCAAACAUGUACGGCCUGAACAAGGAG
GUGGUCAGGCAGCUGAGCAGGCGGUACCCACAGCUGCCCAGGGCCGUGGCCAC
CGGCAGGGUGUACGACAUGAACACCGGCACCCUGAGGAACUACGACCCCAGGA
UCAACCUGGUGCCCGUGAACAGGCGGCUGCCCCACGCCCUGGUGCUGCACCAC
AACGAGCACCCACAGAGCGACUUCAGCUCCUUCGUGAGCAAGCUGAAAGGCAG
GACCGUGCUGGUCGUGGGCGAGAAGCUGAGCGUGCCCGGCAAGAUGGUGGAC
UGGCUGAGCGACAGGCCCGAGGCCACCUUCCGGGCCAGGCUGGACCUCGGCAU
CCCCGGCGACGUGCCCAAGUACGACAUCAUCUUCGUGAACGUCAGGACCCCAU
ACAAGUACCACCAUUACCAGCAGUGCGAGGACCACGCCAUCAAGCUGAGCAUG
CUGACCAAGAAGGCCUGCCUGCACCUGAACCCCGGAGGCACCUGCGUGAGCAU
CGGCUACGGCUACGCCGACAGGGCCAGCGAGAGCAUCAUUGGCGCCAUCGCCA
GGCUGUUCAAGUUCAGCAGGGUGUGCAAACCCAAGAGCAGCCUGGAGGAAACC
GAGGUGCUGUUCGUGUUCAUCGGCUACGACCGGAAGGCCAGGACCCACAACCC
CUACAAGCUGAGCAGCACCCUGACAAACAUCUACACCGGCAGCAGGCUGCACG
AGGCCGGCUGCGCCCCCAGCUACCACGUGGUCAGGGGCGAUAUCGCCACCGCC
ACCGAGGGCGUGAUCAUCAACGCUGCCAACAGCAAGGGCCAGCCCGGAGGCGG
AGUGUGCGGCGCCCUGUACAAGAAGUUCCCCGAGAGCUUCGACCUGCAGCCCA
UCGAGGUGGGCAAGGCCAGGCUGGUGAAGGGCGCCGCUAAGCACAUCAUCCAC
GCCGUGGGCCCCAACUUCAACAAGGUGAGCGAGGUGGAAGGCGACAAGCAGCU
GGCCGAAGCCUACGAGAGCAUCGCCAAGAUCGUGAACGACAAUAACUACAAGA
GCGUGGCCAUCCCACUGCUCAGCACCGGCAUCUUCAGCGGCAACAAGGACAGG
CUGACCCAGAGCCUGAACCACCUGCUCACCGCCCUGGACACCACCGAUGCCGA
CGUGGCCAUCUACUGCAGGGACAAGAAGUGGGAGAUGACCCUGAAGGAGGCC
GUGGCCAGGCGGGAGGCCGUGGAAGAGAUCUGCAUCAGCGACGACUCCAGCGU
GACCGAGCCCGACGCCGAGCUGGUGAGGGUGCACCCCAAGAGCUCCCUGGCCG
GCAGGAAGGGCUACAGCACCAGCGACGGCAAGACCUUCAGCUACCUGGAGGGC
ACCAAGUUCCACCAGGCCGCUAAGGACAUCGCCGAGAUCAACGCUAUGUGGCC
CGUGGCCACCGAGGCCAACGAGCAGGUGUGCAUGUACAUCCUGGGCGAGAGCA
UGUCCAGCAUCAGGAGCAAGUGCCCCGUGGAGGAAAGCGAGGCCAGCACACCA
CCCAGCACCCUGCCCUGCCUGUGCAUCCACGCUAUGACACCCGAGAGGGUGCA
GCGGCUGAAGGCCAGCAGGCCCGAGCAGAUCACCGUGUGCAGCUCCUUCCCAC
UGCCCAAGUACAGGAUCACCGGCGUGCAGAAGAUCCAGUGCAGCCAGCCCAUC
CUGUUCAGCCCAAAGGUGCCCGCCUACAUCCACCCCAGGAAGUACCUGGUGGA
GACCCCACCCGUGGACGAGACACCCGAGCCAAGCGCCGAGAACCAGAGCACCG
AGGGCACACCCGAGCAGCCACCCCUGAUCACCGAGGACGAGACAAGGACCCGG
ACCCCAGAGCCCAUCAUUAUCGAGGAAGAGGAAGAGGACAGCAUCAGCCUGCU
GAGCGACGGCCCCACCCACCAGGUGCUGCAGGUGGAGGCCGACAUCCACGGCC
CACCCAGCGUGUCCAGCUCCAGCUGGAGCAUCCCACACGCCAGCGACUUCGAC
GUGGACAGCCUGAGCAUCCUGGACACCCUGGAGGGCGCCAGCGUGACCUCCGG
CGCCACCAGCGCCGAGACCAACAGCUACUUCGCCAAGAGCAUGGAGUUCCUGG
CCAGGCCCGUGCCAGCUCCCAGGACCGUGUUCAGGAACCCACCCCACCCAGCUC
CCAGGACCAGGACCCCAAGCCUGGCUCCCAGCAGGGCCUGCAGCAGGACCAGC
CUGGUGAGCACCCCACCCGGCGUGAACAGGGUGAUCACCAGGGAGGAACUGGA
GGCCCUGACACCCAGCAGGACCCCCAGCAGGUCCGUGAGCAGGACUAGUCUGG
UGUCCAACCCACCCGGCGUGAACAGGGUGAUCACCAGGGAGGAAUUCGAGGCC
UUCGUGGCCCAGCAACAGAGACGGUUCGACGCCGGCGCCUACAUCUUCAGCAG
CGACACCGGCCAGGGACACCUGCAGCAAAAGAGCGUGAGGCAGACCGUGCUGA
GCGAGGUGGUGCUGGAGAGGACCGAGCUGGAAAUCAGCUACGCCCCCAGGCUG
GACCAGGAGAAGGAGGAACUGCUCAGGAAGAAACUGCAGCUGAACCCCACCCC
AGCCAACAGGAGCAGGUACCAGAGCAGGAAGGUGGAGAACAUGAAGGCCAUC
ACCGCCAGGCGGAUCCUGCAGGGCCUGGGACACUACCUGAAGGCCGAGGGCAA
GGUGGAGUGCUACAGGACCCUGCACCCCGUGCCACUGUACAGCUCCAGCGUGA
ACAGGGCCUUCUCCAGCCCCAAGGUGGCCGUGGAGGCCUGCAACGCUAUGCUG
AAGGAGAACUUCCCCACCGUGGCCAGCUACUGCAUCAUCCCCGAGUACGACGC
CUACCUGGACAUGGUGGACGGCGCCAGCUGCUGCCUGGACACCGCCAGCUUCU
GCCCCGCCAAGCUGAGGAGCUUCCCCAAGAAACACAGCUACCUGGAGCCCACC
AUCAGGAGCGCCGUGCCCAGCGCCAUCCAGAACACCCUGCAGAACGUGCUGGC
CGCUGCCACCAAGAGGAACUGCAACGUGACCCAGAUGAGGGAGCUGCCCGUGC
UGGACAGCGCUGCCUUCAACGUGGAGUGCUUCAAGAAAUACGCCUGCAACAAC
GAGUACUGGGAGACCUUCAAGGAGAACCCCAUCAGGCUGACCGAAGAGAACGU
GGUGAACUACAUCACCAAGCUGAAGGGCCCCAAGGCCGCUGCCCUGUUCGCUA
AGACCCACAACCUGAACAUGCUGCAGGACAUCCCAAUGGACAGGUUCGUGAUG
GACCUGAAGAGGGACGUGAAGGUGACACCCGGCACCAAGCACACCGAGGAGAG
GCCCAAGGUGCAGGUGAUCCAGGCCGCUGACCCACUGGCCACCGCCUACCUGU
GCGGCAUCCACAGGGAGCUGGUGAGGCGGCUGAACGCCGUGCUGCUGCCCAAC
AUCCACACCCUGUUCGACAUGAGCGCCGAGGACUUCGACGCCAUCAUCGCCGA
GCACUUCCAGCCCGGCGACUGCGUGCUGGAGACCGACAUCGCCAGCUUCGACA
AGAGCGAGGAUGACGCUAUGGCCCUGACCGCUCUGAUGAUCCUGGAGGACCUG
GGCGUGGACGCCGAGCUGCUCACCCUGAUCGAGGCUGCCUUCGGCGAGAUCAG
CUCCAUCCACCUGCCCACCAAGACCAAGUUCAAGUUCGGCGCUAUGAUGAAAA
GCGGAAUGUUCCUGACCCUGUUCGUGAACACCGUGAUCAACAUUGUGAUCGCC
AGCAGGGUGCUGCGGGAGAGGCUGACCGGCAGCCCCUGCGCUGCCUUCAUCGG
CGACGACAACAUCGUGAAGGGCGUGAAAAGCGACAAGCUGAUGGCCGACAGG
UGCGCCACCUGGCUGAACAUGGAGGUGAAGAUCAUCGACGCCGUGGUGGGCGA
GAAGGCCCCCUACUUCUGCGGCGGAUUCAUCCUGUGCGACAGCGUGACCGGCA
CCGCCUGCAGGGUGGCCGACCCCCUGAAGAGGCUGUUCAAGCUGGGCAAGCCA
CUGGCCGCUGACGAUGAGCACGACGAUGACAGGCGGAGGGCCCUGCACGAGGA
AAGCACCAGGUGGAACAGGGUGGGCAUCCUGAGCGAGCUGUGCAAGGCCGUG
GAGAGCAGGUACGAGACCGUGGGCACCAGCAUCAUCGUGAUGGCUAUGACCAC
ACUGGCCAGCUCCGUCAAGAGCUUCUCCUACCUGAGGGGGGCCCCUAUAACUC
UCUACGGCUAACCUGAAUGGACUACGACAUAGUCUAGUCCGCCAAGGCCGCCA
CCAUGGAAGAUGCCAAAAACAUUAAGAAGGGCCCAGCGCCAUUCUACCCACUC
GAAGACGGGACCGCCGGCGAGCAGCUGCACAAAGCCAUGAAGCGCUACGCCCU
GGUGCCCGGCACCAUCGCCUUUACCGACGCACAUAUCGAGGUGGACAUUACCU
ACGCCGAGUACUUCGAGAUGAGCGUUCGGCUGGCAGAAGCUAUGAAGCGCUA
UGGGCUGAAUACAAACCAUCGGAUCGUGGUGUGCAGCGAGAAUAGCUUGCAG
UUCUUCAUGCCCGUGUUGGGUGCCCUGUUCAUCGGUGUGGCUGUGGCCCCAGC
UAACGACAUCUACAACGAGCGCGAGCUGCUGAACAGCAUGGGCAUCAGCCAGC
CCACCGUCGUAUUCGUGAGCAAGAAAGGGCUGCAAAAGAUCCUCAACGUGCAA
AAGAAGCUACCGAUCAUACAAAAGAUCAUCAUCAUGGAUAGCAAGACCGACU
ACCAGGGCUUCCAAAGCAUGUACACCUUCGUGACUUCCCAUUUGCCACCCGGC
UUCAACGAGUACGACUUCGUGCCCGAGAGCUUCGACCGGGACAAAACCAUCGC
CCUGAUCAUGAACAGUAGUGGCAGUACCGGAUUGCCCAAGGGCGUAGCCCUAC
CGCACCGCACCGCUUGUGUCCGAUUCAGUCAUGCCCGCGACCCCAUCUUCGGC
AACCAGAUCAUCCCCGACACCGCUAUCCUCAGCGUGGUGCCAUUUCACCACGG
CUUCGGCAUGUUCACCACGCUGGGCUACUUGAUCUGCGGCUUUCGGGUCGUGC
UCAUGUACCGCUUCGAGGAGGAGCUAUUCUUGCGCAGCUUGCAAGACUAUAA
GAUUCAAUCUGCCCUGCUGGUGCCCACACUAUUUAGCUUCUUCGCUAAGAGCA
CUCUCAUCGACAAGUACGACCUAAGCAACUUGCACGAGAUCGCCAGCGGGGG
GCGCCGCUCAGCAAGGAGGUAGGUGAGGCCGUGGCCAAACGCUUCCACCUACC
AGGCAUCCGACAGGGCUACGGCCUGACAGAAACAACCAGCGCCAUUCUGAUCA
CCCCCGAAGGGGACGACAAGCCUGGCGCAGUAGGCAAGGUGGUGCCCUUCUUC
GAGGCUAAGGUGGUGGACUUGGACACCGGUAAGACACUGGGUGUGAACCAGC
GCGGCGAGCUGUGCGUCCGUGGCCCCAUGAUCAUGAGCGGCUACGUUAACAAC
CCCGAGGCUACAAACGCUCUCAUCGACAAGGACGGCUGGCUGCACAGCGGCGA
CAUCGCCUACUGGGACGAGGACGAGCACUUCUUCAUCGUGGACCGGCUGAAGU
CCCUGAUCAAAUACAAGGGCUACCAGGUAGCCCCAGCCGAACUGGAGAGCAUC
CUGCUGCAACACCCCAACAUCUUCGACGCCGGGGUCGCCGGCCUGCCCGACGA
CGAUGCCGGCGAGCUGCCCGCCGCAGUCGUCGUGCUGGAACACGGUAAAACCA
UGACCGAGAAGGAGAUCGUGGACUAUGUGGCCAGCCAGGUUACAACCGCCAAG
AAGCUGCGCGGUGGUGUUGUGUUCGUGGACGAGGUGCCUAAAGGACUGACCG
GCAAGUUGGACGCCCGCAAGAUCCGCGAGAUUCUCAUUAAGGCCAAGAAGGGC
GGCAAGAUCGCCGUGUAACUCGAGCCGGAAACGCAAUAGCCGAAAAACAAAAA
ACAAAAAAAACAAAAAAAAAACCAAAAAAACAAAACACAUUAAAACAGCCUG
UGGGUUGAUCCCACCCACAGGCCCAUUGGGCGCUAGCACUCUGGUAUCACGGU
ACCUUUGUGCGCCUGUUUUAUACCCCCUCCCCCAACUGUAACUUAGAAGUAAC
ACACACCGAUCAACAGUCAGCGUGGCACACCAGCCACGUUUUGAUCAAGCACU
UCUGUUACCCCGGACUGAGUAUCAAUAGACUGCUCACGCGGUUGAAGGAGAA
AGCGUUCGUUAUCCGGCCAACUACUUCGAAAAACCUAGUAACACCGUGGAAGU
UGCAGAGUGUUUCGCUCAGCACUACCCCAGUGUAGAUCAGGUCGAUGAGUCAC
CGCAUUCCCCACGGGCGACCGUGGCGGUGGCUGCGUUGGCGGCCUGCCCAUGG
GGAAACCCAUGGGACGCUCUAAUACAGACAUGGUGCGAAGAGUCUAUUGAGC
UAGUUGGUAGUCCUCCGGCCCCUGAAUGCGGCUAAUCCUAACUGCGGAGCACA
CACCCUCAAGCCAGAGGGCAGUGUGUCGUAACGGGCAACUCUGCAGCGGAACC
GACUACUUUGGGUGUCCGUGUUUCAUUUUAUUCCUAUACUGGCUGCUUAUGG
UGACAAUUGAGAGAUCGUUACCAUAUAGCUAUUGGAUUGGCCAUCCGGUGAC
UAAUAGAGCUAUUAUAUAUCCCUUUGUUGGGUUUAUACCACUUAGCUUGAAA
GAGGUUAAAACAUUACAAUUCAUUGUUAAGUUGAAUACAGCAAAAUGAGCAA
GAUCUACAUCGACGAGCGGAGCAACGCCGAGAUCGUGUGCGAGGCCAUCAAGA
CCAUCGGCAUCGAGGGCGCCACCGCCGCCCAGCUGACCAGGCAGCUGAACAUG
GAGAAGCGGGAGGUGAACAAGGCCCUGUACGACCUGCAGAGGAGCGCUAUGG
UGUACUCCAGCGACGACAUCCCUCCCCGGUGGUUCAUGACCACCGAGGCCGAC
AAGCCCGACGCCGACGCUAUGGCCGACGUGAUCAUCGACGACGUGAGCAGGGA
GAAGUCCAUGAGGGAGGACCACAAGAGCUUCGACGACGUGAUCCCCGCCAAGA
AGAUCAUCGACUGGAAGGGCGCCAACCCCGUGACCGUGAUCAACGAGUACUGC
CAGAUCACCAGGAGGGACUGGAGCUUCCGGAUCGAGAGCGUGGGCCCCAGCAA
CAGCCCCACCUUCUACGCCUGCGUGGACAUCGACGGCAGGGUGUUCGACAAGG
CCGACGGCAAGAGCAAGCGGGACGCCAAGAACAACGCCGCCAAGCUGGCCGUG
GACAAGCUGCUGGGCUACGUGAUCAUCCGGUUCUAAACAAUUGGCAAGCUGCU
UACAUAGAACUCGCGGCGAUUGGCAUGCCGCCUUAAAAUUUUUAUUUUAUUU
UUUCUUUUCUUUUCCGAAUCGGAUUUUGUUUUUAAUAUUUCAAAAAAAAAAA
AAAAAAAAAAAAAAUCUAGAAAAAAAAAAAAAAAAAAAAAAAAAAAAAAAAA
AAAAAAAAAAAAAAAAAAAAAAAAAAAAAAAAAAAAAAAAAAAAAAAAAAAA
AAAAAAAAAAAAAAA

TABLE 8

| SEQ ID NO | Description |
|---|---|
| SEQ ID NO: 72 | nsP1-4 ORF, codon-optimized |
| SEQ ID NO: 73 | 5′ UTR |
| SEQ ID NO: 74 | 5′ UTR |
| SEQ ID NO: 75 | 5′ UTR |
| SEQ ID NO: 76 | 3′ UTR |
| SEQ ID NO: 121 | SARS-CoV-2 spike glycoprotein |
| | (non-codon optimized nucleic acid) |
| SEQ ID NO: 122 | SARS-CoV-2 spike glycoprotein |
| | (codon-optimized nucleic acid) |
| SEQ ID NO: 123 | SARS-CoV-2 spike glycoprotein |
| | (wild-type protein) |
| SEQ ID NO: 77 | Intergenic region between nsP1-4 |
| | ORF and antigenic protein ORF |
| SEQ ID NO: 78 | Replicon sequence comprising SEQ ID |
| | NO: 72, SEQ ID NO: 73, SEQ |
| | ID NO: 76, and SEQ ID NO: 77 |
| SEQ ID NO: 124 | Replicon sequence comprising |
| | SEQ ID NO: 72, SEQ ID NO: 73, |
| | SEQ ID NO: 76, SEQ ID NO: 121, |
| | and SEQ ID NO: 77 |
| SEQ ID NO: 125 | Replicon sequence comprising |
| | SEQ ID NO: 72, SEQ ID NO: 73, |
| | SEQ ID NO: 76, SEQ ID NO: 122, |
| | and SEQ ID NO: 77 (ARCT-021; aka |
| | “STARR ™ SARS-CoV-2 RNA) |
| SEQ ID NO: 79 | nsP1-4 protein sequence |
| SEQ ID NO: 80 | nsP1-4 protein sequence |
| SEQ ID NO: 81 | nsP1-4 protein sequence |
| SEQ ID NO: 126 | mRNA encoding SARS- |
| | CoV-2 glycoprotein |
| SEQ ID NO: 82 | 5′ UTR (TEV) |
| SEQ ID NO: 83 | 3′ UTR (Xbg) |

### Example 10

This example describes characterization of self-replicating (STARR™) technology using firefly luciferase transgene expression.

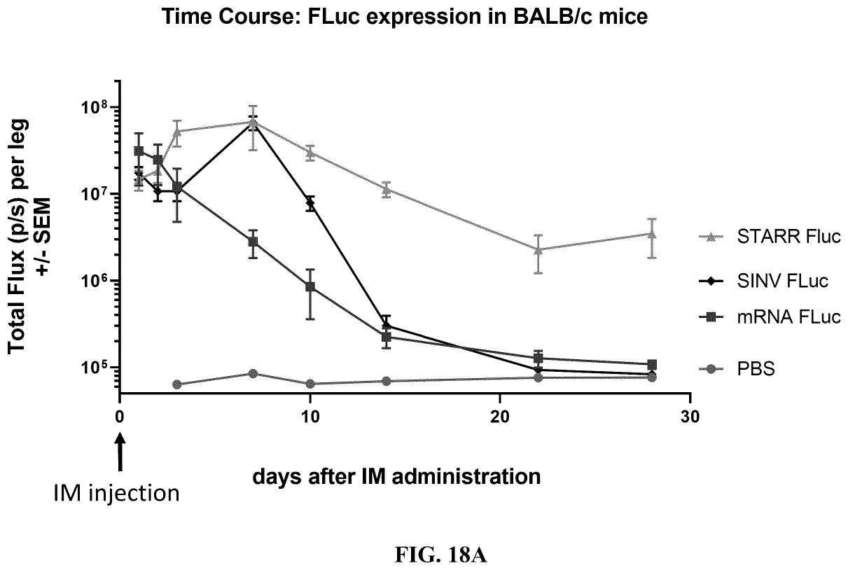

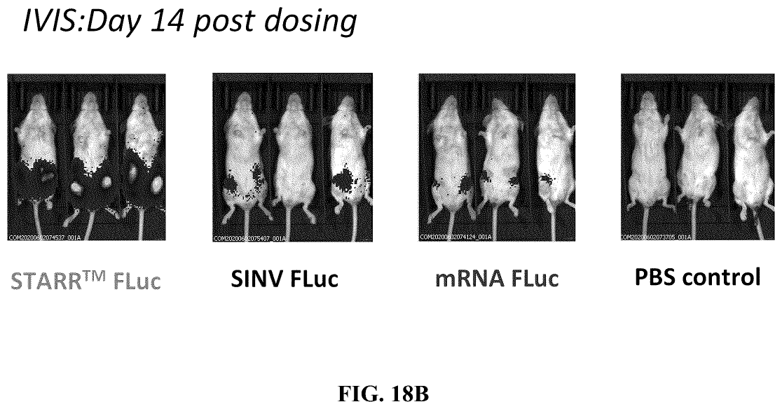

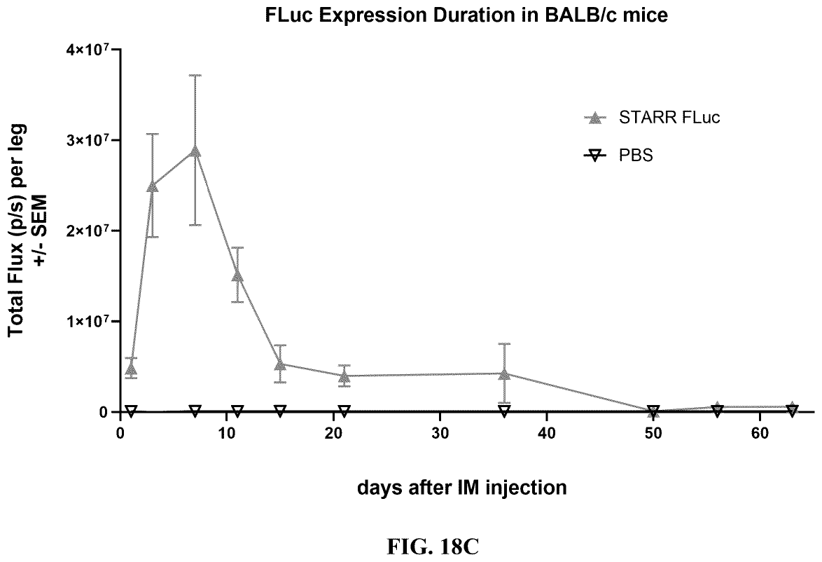

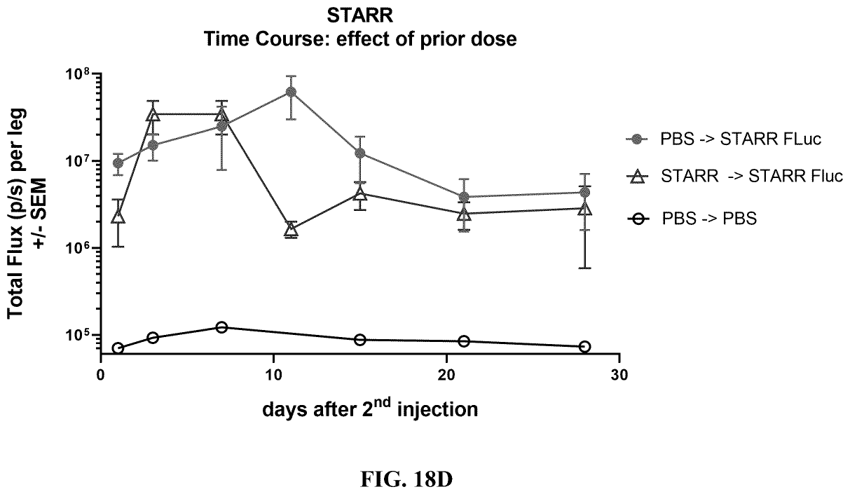

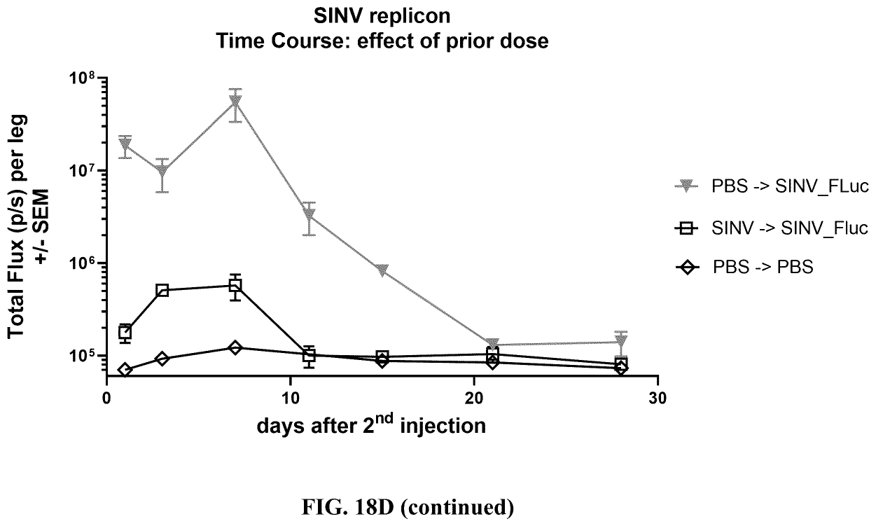

In vitro transcripts were formulated with lipid nanoparticles (LNP) at a concentration of 0.1 mg/ml, and injected intramuscularly in both legs of female BALB/C mice (n=3) at a dose of 5 ug per leg. Expression of firefly luciferase (FLuc) was measured by IVIS Lumina LT Series III (PerkinElmer) by administering 100 ul of 1.5 mg Xenolight D-luciferin (PerkinElmer) in PBS via intraperitoneal injection ˜10 min prior to the measurement. Six data points per group of mice were obtained at each time point (FIGS. 18A-18D).

Firefly luciferase (FLuc) expression was monitored from STARR™ Fluc, SINV FLuc, and mRNA FLuc up to day 28 by In Vivo Imaging System (IVIS). Enhanced levels and durations of transgene expression from STARR™ were observed. The expression from STARR™ Fluc peaked around day 3 to 7 and declined until day 22. Fluc expression from SINV FLuc also peaked on day 10, however, the expression was reduced at a significantly faster rate than STARR™ FLuc. Additionally, the expression on day 3 was significantly lower than STARR™ FLuc. FLuc expression from the conventional mRNA backbone was highest at day 1, the earliest time point in this study, and declined at a slightly faster rate than that of STARR™-Fluc (FIG. 18A). FIG. 18B shows that at 14 days post dosing, FLuc expression from STARR™ FLuc was higher than the other groups by about two orders of magnitude. FIG. 18D shows that the effect of the STARR™ backbone remained minimal throughout the experimental period (up to day 28), while prior administration of SINV replicon backbone resulted in a reduction of FLuc transgene expression by ˜2 orders of magnitude.

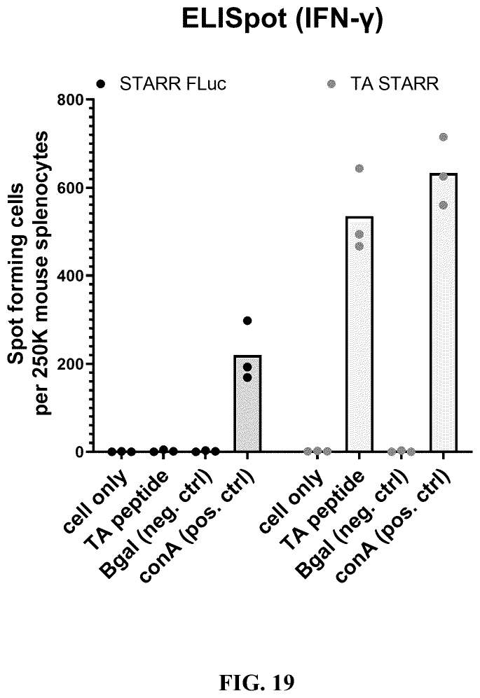

A cancer vaccine substrate, TA STARR™, was constructed next with the STARR™ backbone that encodes AH1A5 epitope from gp70, an envelope glycoprotein of endogenous Murine leukemia virus. AH1 (SPSYVYHQF) (SEQ ID NO:110) is an H-2Ld-restricted antigen of gp70423-431, which is expressed in tumor cells such as the CT26 colorectal cancer cell line, but not expressed in most of the normal tissues. AH1-A5 is a mutated sequence with SPSYAYHQF (SEQ ID NO:111) (the mutation underlined) with enhanced affinity to the T cell receptor (Slansky, et al., 2000, Immunity 13: 529-538). The open reading frame of the TA STARR™ subgenomic RNA contains a cassette with a signal peptide from the HLA class I antigen, gp70 sequence containing AH1A5 epitope, ovalbumin epitope (OVA323-339), and MHC class I trafficking signal (Kreiter, et al. 2008, J Immunol 180: 309-318). Three female BALB/c mice were intramuscularly injected with 10 ug of LNP formulated STARR™ transcripts, STARR™ FLuc or TA STARR™, on day 0 and day 7. On day 16, the spleens were harvested and the splenocytes were isolated. Splenocytes (2.5×105 cells) were incubated with or without AHIA5 (SPSYAYHQF) (SEQ ID NO:111), beta-gal peptide (TPHPARIGL) (SEQ ID NO:112) at 1 ug/ml, and lx Concanavalin A (Life Technologies). ELISpot detecting murine IFN-gamma (ImmunoSpot) was performed according to the manufacturer's instructions. As can be seen in FIG. 19, TA STARR™ elicited antigen-specific IFN-gamma responses.

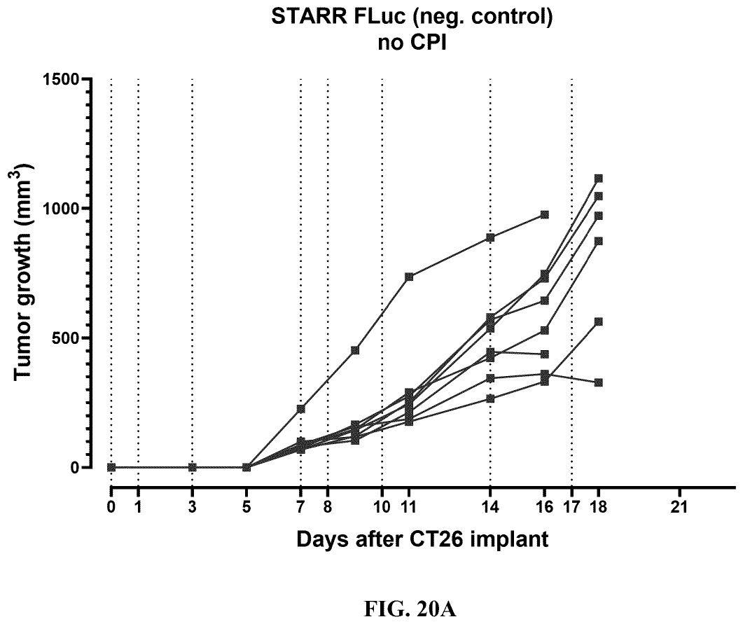

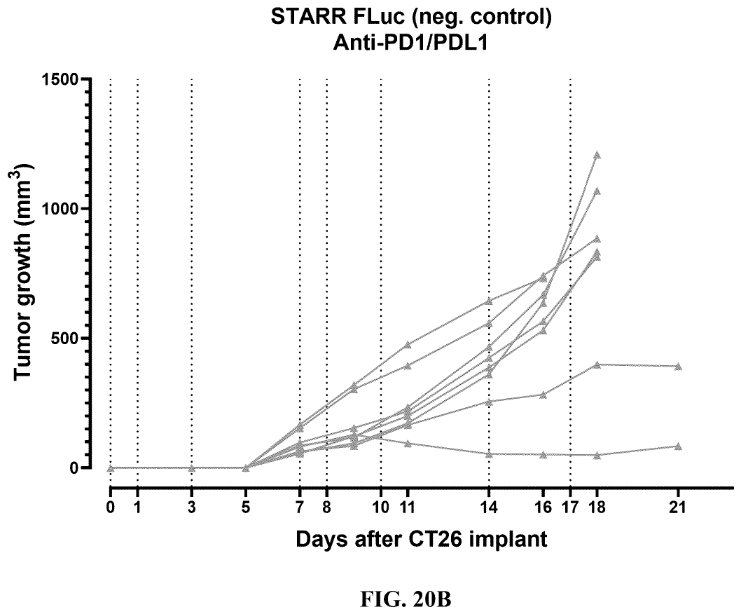

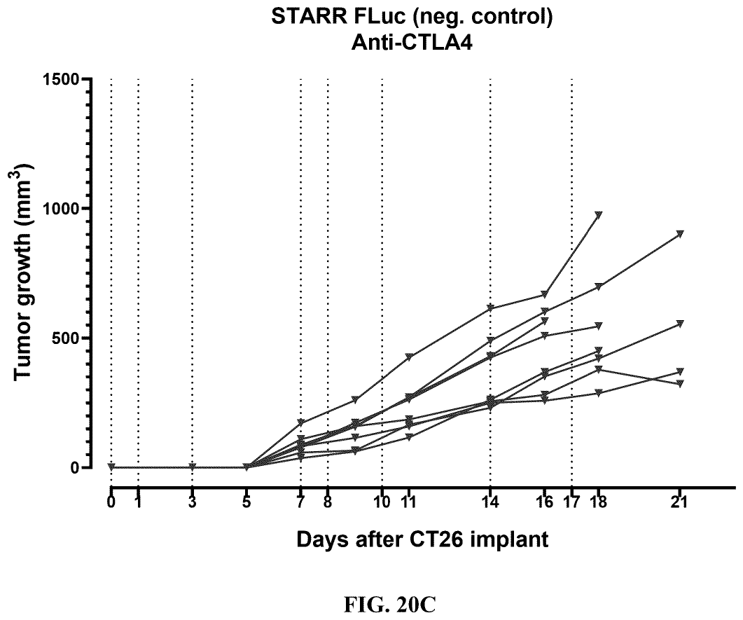

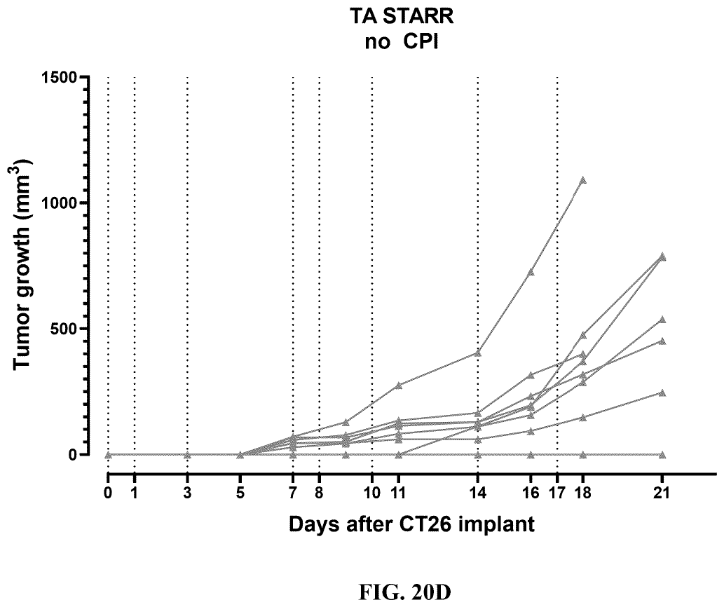

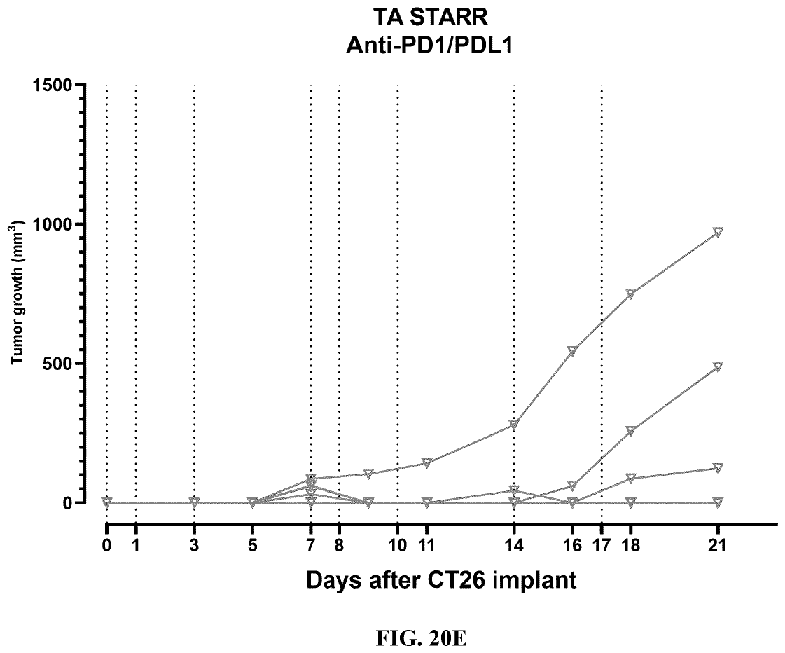

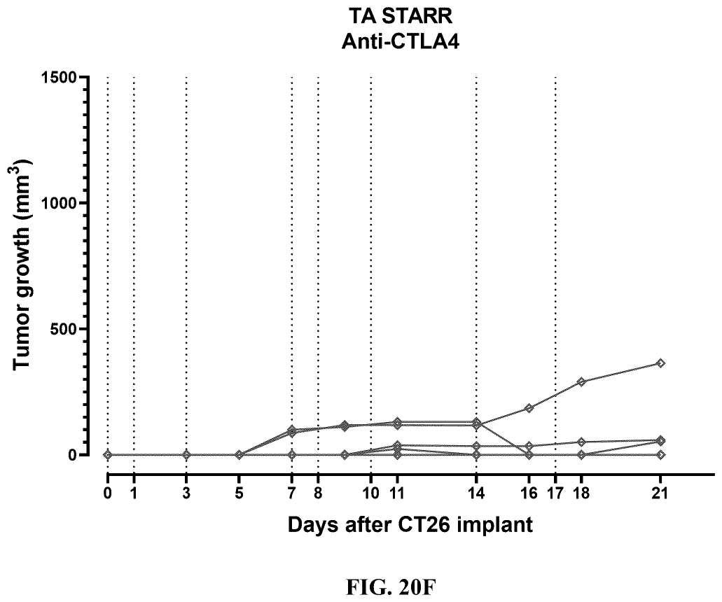

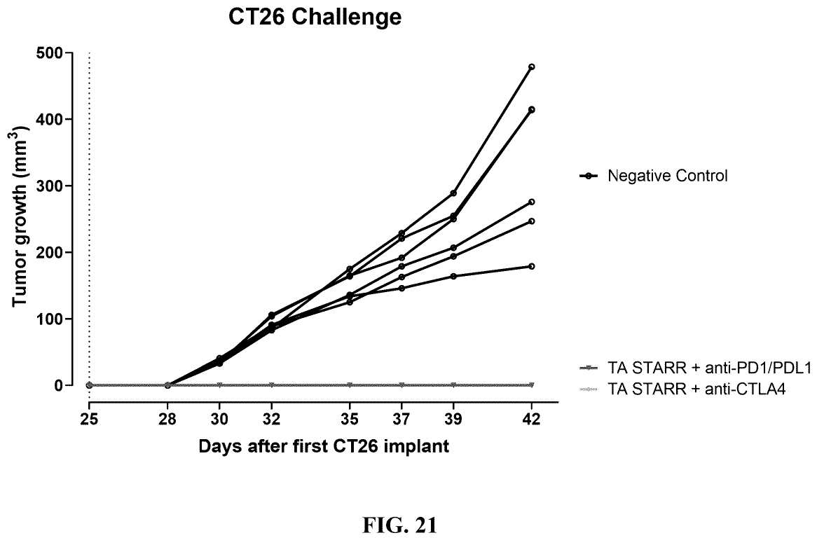

BALB/c mice, 10 week-old female, were subcutaneously implanted in the right flank with 5×105 cells of CT26 cells in PBS. A day later, LNP-formulated STARR™ RNA was injected intramuscularly in the left leg at a dose of 10 ug in 100 ul. The mice were administered another booster shot on day 8 with the same dose. For a group with combination treatment of anti-mouse PD1 (RMP1-14, BioXCell) and anti-mouse PDL1 (10F.9G2, BioXcell), the combined checkpoint inhibitor (100 ug each) was administered via intraperitoneal injection in the right quadrant twice weekly for two weeks starting on day 3. For a group with the treatment of anti-mouse CTLA4 (9H10, BioxCell), 200 ug of the checkpoint inhibitor was administered in the same manner but starting on day 7. Five mice of the group with the combo treatment of TA STARR™ vaccine and the checkpoint inhibitors remained tumor-free on day 25, and were further challenged by subcutaneous implantation of CT26 (5×105 cells) in the right flank where the implantation site was slightly above the first implantation site. Naïve mice were used as a control group. The tumor growth was monitored for another 17 days (i.e. up to day 42 since the first CT26 implantation) before euthanization. FIGS. 20A-20F illustrates reduced tumor growth resulting from TA STARR™ vaccination and FIG. 21 shows prolonged protection resulting from treatment with the TA STARR™ vaccine in combination with checkpoint inhibitors.

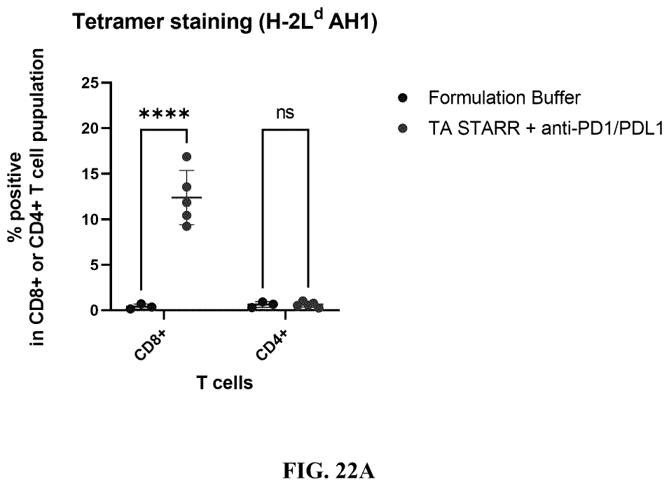

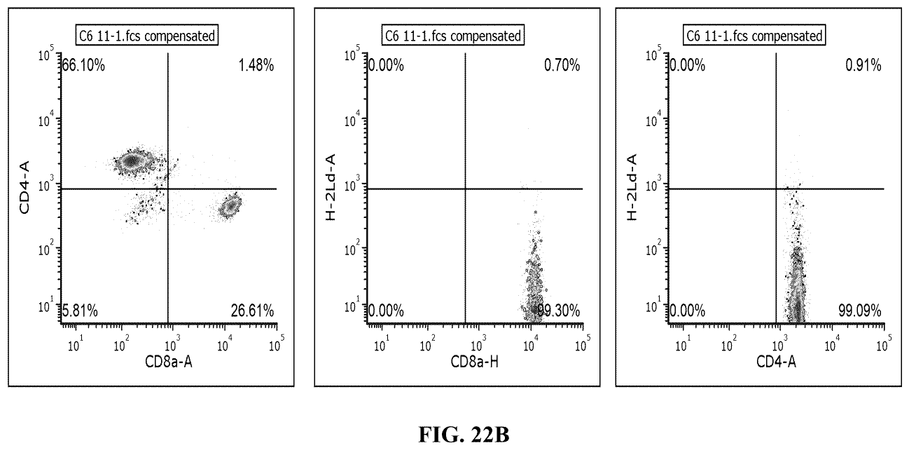

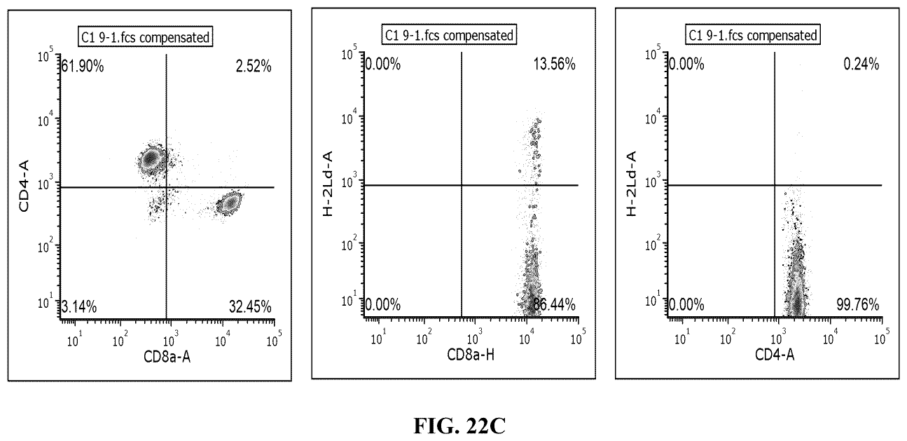

Splenocytes from the combination treatment group with TA STARR™ and anti-PD1/PDL1 were harvested for tetramer staining with AH1 peptide. Splenocytes from the control group with the LNP formulation buffer with the same dosing schedule were used as a negative control. The splenocytes (2×106 cells) were incubated with AH1 (H-2Ld)-tetramer (MBL) followed by appropriate fluorescent-labeled antibodies (Alexa Fluor 488 anti-CD8a (53-6.7), Pacific Orange anti-CD4 (RM4-5), and Pacific Blue anti-mouse CD3F (145-2C11), eBioscience) and DRAQ7 (Invitrogen) by following the manufacture's recommendation, and 500K events were analyzed by ZE5 Cell Analyzer (Bio-Rad). Results are shown in FIGS. 22A-22C.

TABLE 9
Transgene ORF nucleotide sequence

| | RNA | | |
|---|---|---|---|
| mARM | back | | |
| # | bone | Transgene | Sequence |
| 2809 | STARR™ | Fluc | AUGGAAGAUGCCAAAAACAUUAAGAAGGGCCCAGCGCCAUUCUACC |
| (SEQ | | | CACUCGAAGACGGGACCGCCGGCGAGCAGCUGCACAAAGCCAUGAA |
| ID | | | GCGCUACGCCCUGGUGCCCGGCACCAUCGCCUUUACCGACGCACAU |
| NO: 84) | | | AUCGAGGUGGACAUUACCUACGCCGAGUACUUCGAGAUGAGCGUUC |
| | | | GGCUGGCAGAAGCUAUGAAGCGCUAUGGGCUGAAUACAAACCAUCG |
| | | | GAUCGUGGUGUGCAGCGAGAAUAGCUUGCAGUUCUUCAUGCCCGUG |
| | | | UUGGGUGCCCUGUUCAUCGGUGUGGCUGUGGCCCCAGCUAACGACA |
| | | | UCUACAACGAGCGCGAGCUGCUGAACAGCAUGGGCAUCAGCCAGCC |
| | | | CACCGUCGUAUUCGUGAGCAAGAAAGGGCUGCAAAAGAUCCUCAAC |
| | | | GUGCAAAAGAAGCUACCGAUCAUACAAAAGAUCAUCAUCAUGGAUA |
| | | | GCAAGACCGACUACCAGGGCUUCCAAAGCAUGUACACCUUCGUGAC |
| | | | UUCCCAUUUGCCACCCGGCUUCAACGAGUACGACUUCGUGCCCGAG |
| | | | AGCUUCGACCGGGACAAAACCAUCGCCCUGAUCAUGAACAGUAGUG |
| | | | GCAGUACCGGAUUGCCCAAGGGCGUAGCCCUACCGCACCGCACCGC |
| | | | UUGUGUCCGAUUCAGUCAUGCCCGCGACCCCAUCUUCGGCAACCAG |
| | | | AUCAUCCCCGACACCGCUAUCCUCAGCGUGGUGCCAUUUCACCACG |
| | | | GCUUCGGCAUGUUCACCACGCUGGGCUACUUGAUCUGCGGCUUUCG |
| | | | GGUCGUGCUCAUGUACCGCUUCGAGGAGGAGCUAUUCUUGCGCAGC |
| | | | UUGCAAGACUAUAAGAUUCAAUCUGCCCUGCUGGUGCCCACACUAU |
| | | | UUAGCUUCUUCGCUAAGAGCACUCUCAUCGACAAGUACGACCUAAG |
| | | | CAACUUGCACGAGAUCGCCAGCGGCGGGGCGCCGCUCAGCAAGGAG |
| | | | GUAGGUGAGGCCGUGGCCAAACGCUUCCACCUACCAGGCAUCCGAC |
| | | | AGGGCUACGGCCUGACAGAAACAACCAGCGCCAUUCUGAUCACCCC |
| | | | CGAAGGGGACGACAAGCCUGGCGCAGUAGGCAAGGUGGUGCCCUUC |
| | | | UUCGAGGCUAAGGUGGUGGACUUGGACACCGGUAAGACACUGGGUG |
| | | | UGAACCAGCGCGGCGAGCUGUGCGUCCGUGGCCCCAUGAUCAUGAG |
| | | | CGGCUACGUUAACAACCCCGAGGCUACAAACGCUCUCAUCGACAAG |
| | | | GACGGCUGGCUGCACAGCGGCGACAUCGCCUACUGGGACGAGGACG |
| | | | AGCACUUCUUCAUCGUGGACCGGCUGAAGUCCCUGAUCAAAUACAA |
| | | | GGGCUACCAGGUAGCCCCAGCCGAACUGGAGAGCAUCCUGCUGCAA |
| | | | CACCCCAACAUCUUCGACGCCGGGGUCGCCGGCCUGCCCGACGACG |
| | | | AUGCCGGCGAGCUGCCCGCCGCAGUCGUCGUGCUGGAACACGGUAA |
| | | | AACCAUGACCGAGAAGGAGAUCGUGGACUAUGUGGCCAGCCAGGUU |
| | | | ACAACCGCCAAGAAGCUGCGCGGUGGUGUUGUGUUCGUGGACGAGG |
| | | | UGCCUAAAGGACUGACCGGCAAGUUGGACGCCCGCAAGAUCCGCGA |
| | | | GAUUCUCAUUAAGGCCAAGAAGGGCGGCAAGAUCGCCGUGUAA |
| 2842 | SINV | Fluc | AUGGAAGAUGCCAAAAACAUUAAGAAGGGCCCAGCGCCAUUCUACC |
| (SEQ | repli- | | CACUCGAAGACGGGACCGCCGGCGAGCAGCUGCACAAAGCCAUGAA |
| ID | con | | GCGCUACGCCCUGGUGCCCGGCACCAUCGCCUUUACCGACGCACAU |
| NO: 85) | | | AUCGAGGUGGACAUUACCUACGCCGAGUACUUCGAGAUGAGCGUUC |
| | | | GGCUGGCAGAAGCUAUGAAGCGCUAUGGGCUGAAUACAAACCAUCG |
| | | | GAUCGUGGUGUGCAGCGAGAAUAGCUUGCAGUUCUUCAUGCCCGUG |
| | | | UUGGGUGCCCUGUUCAUCGGUGUGGCUGUGGCCCCAGCUAACGACA |
| | | | UCUACAACGAGCGCGAGCUGCUGAACAGCAUGGGCAUCAGCCAGCC |
| | | | CACCGUCGUAUUCGUGAGCAAGAAAGGGCUGCAAAAGAUCCUCAAC |
| | | | GUGCAAAAGAAGCUACCGAUCAUACAAAAGAUCAUCAUCAUGGAUA |
| | | | GCAAGACCGACUACCAGGGCUUCCAAAGCAUGUACACCUUCGUGAC |
| | | | UUCCCAUUUGCCACCCGGCUUCAACGAGUACGACUUCGUGCCCGAG |
| | | | AGCUUCGACCGGGACAAAACCAUCGCCCUGAUCAUGAACAGUAGUG |
| | | | GCAGUACCGGAUUGCCCAAGGGCGUAGCCCUACCGCACCGCACCGC |
| | | | UUGUGUCCGAUUCAGUCAUGCCCGCGACCCCAUCUUCGGCAACCAG |
| | | | AUCAUCCCCGACACCGCUAUCCUCAGCGUGGUGCCAUUUCACCACG |
| | | | GCUUCGGCAUGUUCACCACGCUGGGCUACUUGAUCUGCGGCUUUCG |
| | | | GGUCGUGCUCAUGUACCGCUUCGAGGAGGAGCUAUUCUUGCGCAGC |
| | | | UUGCAAGACUAUAAGAUUCAAUCUGCCCUGCUGGUGCCCACACUAU |
| | | | UUAGCUUCUUCGCUAAGAGCACUCUCAUCGACAAGUACGACCUAAG |
| | | | CAACUUGCACGAGAUCGCCAGCGGCGGGGCGCCGCUCAGCAAGGAG |
| | | | GUAGGUGAGGCCGUGGCCAAACGCUUCCACCUACCAGGCAUCCGAC |
| | | | AGGGCUACGGCCUGACAGAAACAACCAGCGCCAUUCUGAUCACCCC |
| | | | CGAAGGGGACGACAAGCCUGGCGCAGUAGGCAAGGUGGUGCCCUUC |
| | | | UUCGAGGCUAAGGUGGUGGACUUGGACACCGGUAAGACACUGGGUG |
| | | | UGAACCAGCGCGGCGAGCUGUGCGUCCGUGGCCCCAUGAUCAUGAG |
| | | | CGGCUACGUUAACAACCCCGAGGCUACAAACGCUCUCAUCGACAAG |
| | | | GACGGCUGGCUGCACAGCGGCGACAUCGCCUACUGGGACGAGGACG |
| | | | AGCACUUCUUCAUCGUGGACCGGCUGAAGUCCCUGAUCAAAUACAA |
| | | | GGGCUACCAGGUAGCCCCAGCCGAACUGGAGAGCAUCCUGCUGCAA |
| | | | CACCCCAACAUCUUCGACGCCGGGGUCGCCGGCCUGCCCGACGACG |
| | | | AUGCCGGCGAGCUGCCCGCCGCAGUCGUCGUGCUGGAACACGGUAA |
| | | | AACCAUGACCGAGAAGGAGAUCGUGGACUAUGUGGCCAGCCAGGUU |
| | | | ACAACCGCCAAGAAGCUGCGCGGUGGUGUUGUGUUCGUGGACGAGG |
| | | | UGCCUAAAGGACUGACCGGCAAGUUGGACGCCCGCAAGAUCCGCGA |
| | | | GAUUCUCAUUAAGGCCAAGAAGGGCGGCAAGAUCGCCGUGUAA |
| 1782 | mRNA | Fluc | AUGGAAGAUGCCAAAAACAUUAAGAAGGGCCCAGCGCCAUUCUACC |
| (SEQ | (TEV- | | CACUCGAAGACGGGACCGCCGGCGAGCAGCUGCACAAAGCCAUGAA |
| ID | XbG) | | GCGCUACGCCCUGGUGCCCGGCACCAUCGCCUUUACCGACGCACAU |
| NO: 86) | | | AUCGAGGUGGACAUUACCUACGCCGAGUACUUCGAGAUGAGCGUUC |
| | | | GGCUGGCAGAAGCUAUGAAGCGCUAUGGGCUGAAUACAAACCAUCG |
| | | | GAUCGUGGUGUGCAGCGAGAAUAGCUUGCAGUUCUUCAUGCCCGUG |
| | | | UUGGGUGCCCUGUUCAUCGGUGUGGCUGUGGCCCCAGCUAACGACA |
| | | | UCUACAACGAGCGCGAGCUGCUGAACAGCAUGGGCAUCAGCCAGCC |
| | | | CACCGUCGUAUUCGUGAGCAAGAAAGGGCUGCAAAAGAUCCUCAAC |
| | | | GUGCAAAAGAAGCUACCGAUCAUACAAAAGAUCAUCAUCAUGGAUA |
| | | | GCAAGACCGACUACCAGGGCUUCCAAAGCAUGUACACCUUCGUGAC |
| | | | UUCCCAUUUGCCACCCGGCUUCAACGAGUACGACUUCGUGCCCGAG |
| | | | AGCUUCGACCGGGACAAAACCAUCGCCCUGAUCAUGAACAGUAGUG |
| | | | GCAGUACCGGAUUGCCCAAGGGCGUAGCCCUACCGCACCGCACCGC |
| | | | UUGUGUCCGAUUCAGUCAUGCCCGCGACCCCAUCUUCGGCAACCAG |
| | | | AUCAUCCCCGACACCGCUAUCCUCAGCGUGGUGCCAUUUCACCACG |
| | | | GCUUCGGCAUGUUCACCACGCUGGGCUACUUGAUCUGCGGCUUUCG |
| | | | GGUCGUGCUCAUGUACCGCUUCGAGGAGGAGCUAUUCUUGCGCAGC |
| | | | UUGCAAGACUAUAAGAUUCAAUCUGCCCUGCUGGUGCCCACACUAU |
| | | | UUAGCUUCUUCGCUAAGAGCACUCUCAUCGACAAGUACGACCUAAG |
| | | | CAACUUGCACGAGAUCGCCAGCGGCGGGGCGCCGCUCAGCAAGGAG |
| | | | GUAGGUGAGGCCGUGGCCAAACGCUUCCACCUACCAGGCAUCCGAC |
| | | | AGGGCUACGGCCUGACAGAAACAACCAGCGCCAUUCUGAUCACCCC |
| | | | CGAAGGGGACGACAAGCCUGGCGCAGUAGGCAAGGUGGUGCCCUUC |
| | | | UUCGAGGCUAAGGUGGUGGACUUGGACACCGGUAAGACACUGGGUG |
| | | | UGAACCAGCGCGGCGAGCUGUGCGUCCGUGGCCCCAUGAUCAUGAG |
| | | | CGGCUACGUUAACAACCCCGAGGCUACAAACGCUCUCAUCGACAAG |
| | | | GACGGCUGGCUGCACAGCGGCGACAUCGCCUACUGGGACGAGGACG |
| | | | AGCACUUCUUCAUCGUGGACCGGCUGAAGUCCCUGAUCAAAUACAA |
| | | | GGGCUACCAGGUAGCCCCAGCCGAACUGGAGAGCAUCCUGCUGCAA |
| | | | CACCCCAACAUCUUCGACGCCGGGGUCGCCGGCCUGCCCGACGACG |
| | | | AUGCCGGCGAGCUGCCCGCCGCAGUCGUCGUGCUGGAACACGGUAA |
| | | | AACCAUGACCGAGAAGGAGAUCGUGGACUAUGUGGCCAGCCAGGUU |
| | | | ACAACCGCCAAGAAGCUGCGCGGUGGUGUUGUGUUCGUGGACGAGG |
| | | | UGCCUAAAGGACUGACCGGCAAGUUGGACGCCCGCAAGAUCCGCGA |
| | | | GAUUCUCAUUAAGGCCAAGAAGGGCGGCAAGAUCGCCGUGUAA |
| 2847 | STARR™ | KRAS | AUGAAGUUGGUGGUUGUGGGGGCCGGGGGUGUUGGCAAAAGCGCCC |
| (SEQ | | epitope wt | UUACAAUUUGA |
| ID | | | |
| NO: 87) | | | |
| 2862 | SINV | Empty | AUGGAUCCUAGACGCUACGCCCCAAUGAUCCGACCAGCAAAACUCG |
| (SEQ | repli- | | AUGUACUUCCGAGGAACUGA |
| ID | con | | |
| NO: 88) | | | |
| 3060 | STARR™ | Signal | AUGAGAGUGACAGCCCCUAGAACCUUACUGCUUCUGCUUUGGGGAG |
| (SEQ | | peptide- | CUGUUGCUCUGACAGAGACAUGGGCUGGAUCUCUGAGCGAGGUGAC |
| ID | | gp70 with | CGGCCAGGGCCUGUGCAUCGGCGCCGUGCCCAAGACCCACCAGGUG |
| NO: 89) | | AH1A5- | CUGUGCAACACCACCCAGAAGACCAGCGACGGCAGCUACUACCUGG |
| | | MITD | CCGCUCCCACCGGCACCACCUGGGCCUGCAGCACCGGCCUGACCCC |
| | | | UUGCAUCAGCACCACCAUCCUGAACCUGACCACCGACUACUGCGUG |
| | | | CUGGUGGAGCUGUGGCCCAGGGUGACCUACCACAGCCCCAGCUACG |
| | | | CCUACCACCAGUUCGAGAGGAGGGCCAAGUACAAGAGGGAGCCCGU |
| | | | GAGCCUGACCCUGGCCCUGCUGCUGGGCGGCCUGACAAUGGGCGGC |
| | | | AUCGCCGCCGGCGUGGGCACCGGCACCACCGCCCUGGUGGCCACCC |
| | | | AGCAGUUCCAGCAGCUGCAGGCCGCCAUGCACGACGACCUGAAGGA |
| | | | GGUGGAGAAGUCCAUCACCAACCUGGAGAAGUCCCUGACCAGCCUG |
| | | | AGCGAGGUGGUGCUGCAGAACAGGAGGGGCCUGGACCUGCUGUUCC |
| | | | UGAAGGAGGGCGGCCUGUGCGCCGCCCUGAAGGAGGAGUGCUGCCU |
| | | | GUACGCCGACCACACCGGCCUGGUGAUCGUGGGCAUUGUCGCUGGC |
| | | | CUGGCCGUCCUCGCCGUGGUGGUGAUUGGAGCUGUGGUCGCAGCUG |
| | | | UUAUGUGCAGAAGAAAGUCAUCCGGCGGAAAGGGAGGCUCCUACUC |
| | | | UCAGGCUGCUUCUGCUACAGUGCCUAGAGCUCUUAUGUGUUUAUCU |
| | | | CAGCUGUAA |
| 3061 | STARR™ | Signal | AUGAGAGUGACAGCCCCUAGAACCUUACUGCUUCUGCUUUGGGGAG |
| (SEQ | | peptide- | CUGUUGCUCUGACAGAGACAUGGGCUGGAUCUUACCACAGCCCCAG |
| ID | | AH1A5 OVA- | CUACGCCUACCACCAGUUCGAGAGGGGGGGAGGAGGCUCCGGGGGA |
| NO: 90) | | MITD | GGAGGCUCCCUGAAGAUCAGCCAGGCCGUGCACGCCGCCCACGCCG |
| | | | AGAUCAACGAGGCCGGCCGGGAGGUGAUCGUGGGCAUUGUCGCUGG |
| | | | CCUGGCCGUCCUCGCCGUGGUGGUGAUUGGAGCUGUGGUCGCAGCU |
| | | | GUUAUGUGCAGAAGAAAGUCAUCCGGCGGAAAGGGAGGCUCCUACU |
| | | | CUCAGGCUGCUUCUGCUACAGUGCCUAGAGCUCUUAUGUGUUUAUC |
| | | | UCAGCUGUAA |
| 3076 | STARR™ | Signal | AUGAGAGUGACAGCCCCUAGAACCUUACUGCUUCUGCUUUGGGGAG |
| (SEQ | | peptide- | CUGUUGCUCUGACAGAGACAUGGGCUGGAUCUCUGAGCGAGGUGAC |
| ID | | gp70 with | CGGCCAGGGCCUGUGCAUCGGCGCCGUGCCCAAGACCCACCAGGUG |
| NO: 91) | | AH1A5- | CUGUGCAACACCACCCAGAAGACCAGCGACGGCAGCUACUACCUGG |
| | | MITD-FLAG | CCGCUCCCACCGGCACCACCUGGGCCUGCAGCACCGGCCUGACCCC |
| | | | UUGCAUCAGCACCACCAUCCUGAACCUGACCACCGACUACUGCGUG |
| | | | CUGGUGGAGCUGUGGCCCAGGGUGACCUACCACAGCCCCAGCUACG |
| | | | CCUACCACCAGUUCGAGAGGAGGGCCAAGUACAAGAGGGAGCCCGU |
| | | | GAGCCUGACCCUGGCCCUGCUGCUGGGCGGCCUGACAAUGGGCGGC |
| | | | AUCGCCGCCGGCGUGGGCACCGGCACCACCGCCCUGGUGGCCACCC |
| | | | AGCAGUUCCAGCAGCUGCAGGCCGCCAUGCACGACGACCUGAAGGA |
| | | | GGUGGAGAAGUCCAUCACCAACCUGGAGAAGUCCCUGACCAGCCUG |
| | | | AGCGAGGUGGUGCUGCAGAACAGGAGGGGCCUGGACCUGCUGUUCC |
| | | | UGAAGGAGGGCGGCCUGUGCGCCGCCCUGAAGGAGGAGUGCUGCCU |
| | | | GUACGCCGACCACACCGGCCUGGUGAUCGUGGGCAUUGUCGCUGGC |
| | | | CUGGCCGUCCUCGCCGUGGUGGUGAUUGGAGCUGUGGUCGCAGCUG |
| | | | UUAUGUGCAGAAGAAAGUCAUCCGGCGGAAAGGGAGGCUCCUACUC |
| | | | UCAGGCUGCUUCUGCUACAGUGCCUAGAGCUCUUAUGUGUUUAUCU |
| | | | CAGCUGGGCGGCGGAGGCAGCGACUACAAGGACGACGAUGACAAGU |
| | | | AA |
| 3068 | STARR | Signal | AUGAGAGUGACAGCCCCUAGAACCUUACUGCUUCUGCUUUGGGGAG |
| (SEQ | | peptide- | CUGUUGCUCUGACAGAGACAUGGGCUGGAUCUUACCACAGCCCCAG |
| ID | | AH1A5 OVA- | CUACGCCUACCACCAGUUCGAGAGGGGGGGAGGAGGCUCCGGGGGA |
| NO: 92) | | MITD-FLAG | GGAGGCUCCCUGAAGAUCAGCCAGGCCGUGCACGCCGCCCACGCCG |
| | | | AGAUCAACGAGGCCGGCCGGGAGGUGAUCGUGGGCAUUGUCGCUGG |
| | | | CCUGGCCGUCCUCGCCGUGGUGGUGAUUGGAGCUGUGGUCGCAGCU |
| | | | GUUAUGUGCAGAAGAAAGUCAUCCGGCGGAAAGGGAGGCUCCUACU |
| | | | CUCAGGCUGCUUCUGCUACAGUGCCUAGAGCUCUUAUGUGUUUAUC |
| | | | UCAGCUGGGCGGCGGAGGCAGCGACUACAAGGACGACGAUGACAAG |
| | | | UAA |

Transgene ORF amino acid sequence

| mARM | transgene | |
|---|---|---|
| # | description | Sequence |
| 2809, | Fluc | MEDAKNIKKGPAPFYPLEDGTAGEQLHKAMKRYALVPGTIAFTDAH |
| 2842, | | IEVDITYAEYFEMSVRLAEAMKRYGLNTNHRIVVCSENSLQFFMPV |
| 1782 | | LGALFIGVAVAPANDIYNERELLNSMGISQPTVVFVSKKGLQKILN |
| (SEQ | | VQKKLPIIQKIIIMDSKTDYQGFQSMYTFVTSHLPPGFNEYDFVPE |
| ID | | SFDRDKTIALIMNSSGSTGLPKGVALPHRTACVRFSHARDPIFGNQ |
| NO: 93) | | IIPDTAILSVVPFHHGFGMFTTLGYLICGFRVVLMYRFEEELFLRS |
| | | LQDYKIQSALLVPTLFSFFAKSTLIDKYDLSNLHEIASGGAPLSKE |
| | | VGEAVAKRFHLPGIRQGYGLTETTSAILITPEGDDKPGAVGKVVPF |
| | | FEAKVVDLDTGKTLGVNQRGELCVRGPMIMSGYVNNPEATNALIDK |
| | | DGWLHSGDIAYWDEDEHFFIVDRLKSLIKYKGYQVAPAELESILLQ |
| | | HPNIFDAGVAGLPDDDAGELPAAVVVLEHGKTMTEKEIVDYVASQV |
| | | TTAKKLRGGVVFVDEVPKGLTGKLDARKIREILIKAKKGGKIAV* |
| 2847 | KRAS | MKLVVVGAGGVGKSALTI* |
| (SEQ | epitope wt | |
| ID | | |
| NO: 94) | | |
| 2862 | Empty | MDPRRYAPMIRPAKLDVLPRN* |
| (SEQ | | |
| ID | | |
| NO: 95) | | |
| 3060 | Signal | MRVTAPRTLLLLLWGAVALTETWAGSLSEVTGQGLCIGAVPKTHQV |
| (SEQ | peptide- | LCNTTQKTSDGSYYLAAPTGTTWACSTGLTPCISTTILNLTTDYCV |
| ID | gp70 with | LVELWPRVTYHSPSYAYHQFERRAKYKREPVSLTLALLLGGLTMGG |
| NO: 96) | AH1A5- | IAAGVGTGTTALVATQQFQQLQAAMHDDLKEVEKSITNLEKSLTSL |
| | MITD | SEVVLQNRRGLDLLFLKEGGLCAALKEECCLYADHTGLVIVGIVAG |
| | | LAVLAVVVIGAVVAAVMCRRKSSGGKGGSYSQAASATVPRALMCLS |
| | | QL* |
| 3061 | Signal | MRVTAPRTLLLLLWGAVALTETWAGSYHSPSYAYHQFERGGGGSGG |
| (SEQ | peptide- | GGSLKISQAVHAAHAEINEAGREVIVGIVAGLAVLAVVVIGAVVAA |
| ID | AH1A5 OVA- | VMCRRKSSGGKGGSYSQAASATVPRALMCLSQL* |
| NO: 97) | MITD | |
| 3076 | Signal | MRVTAPRTLLLLLWGAVALTETWAGSLSEVTGQGLCIGAVPKTHQV |
| (SEQ | peptide- | LCNTTQKTSDGSYYLAAPTGTTWACSTGLTPCISTTILNLTTDYCV |
| ID | gp70 with | LVELWPRVTYHSPSYAYHQFERRAKYKREPVSLTLALLLGGLTMGG |
| NO: 98) | AH1A5- | IAAGVGTGTTALVATQQFQQLQAAMHDDLKEVEKSITNLEKSLTSL |
| | MITD-FLAG | SEVVLQNRRGLDLLFLKEGGLCAALKEECCLYADHTGLVIVGIVAG |
| | | LAVLAVVVIGAVVAAVMCRRKSSGGKGGSYSQAASATVPRALMCLS |
| | | QLGGGGSDYKDDDDK* |
| 3068 | Signal | MRVTAPRTLLLLLWGAVALTETWAGSYHSPSYAYHQFERGGGGSGG |
| (SEQ | peptide- | GGSLKISQAVHAAHAEINEAGREVIVGIVAGLAVLAVVVIGAVVAA |
| NO: 99) | AH1A5 OVA- | VMCRRKSSGGKGGSYSQAASATVPRALMCLSQLGGGGSDYKDDDDK |
| | MITD-FLAG | * |

whole RNA sequence

| mARM | brief | | |
|---|---|---|---|
| # | name | | Sequence |
| 2809 | STARR™ | 2809 | AUGGGCGGCGCAUGAGAGAAGCCCAGACCAAUUACCUACCCAAAAU |
| (SEQ | Fluc | | GGAGAAAGUUCACGUUGACAUCGAGGAAGACAGCCCAUUCCUCAGA |
| ID | | | GCUUUGCAGCGGAGCUUCCCGCAGUUUGAGGUAGAAGCCAAGCAGG |
| NO: | | | UCACUGAUAAUGACCAUGCUAAUGCCAGAGCGUUUUCGCAUCUGGC |
| 100) | | | UUCAAAACUGAUCGAAACGGAGGUGGACCCAUCCGACACGAUCCUU |
| | | | GACAUUGGAAGUGCGCCCGCCCGCAGAAUGUAUUCUAAGCACAAGU |
| | | | AUCAUUGUAUCUGUCCGAUGAGAUGUGCGGAAGAUCCGGACAGAUU |
| | | | GUAUAAGUAUGCAACUAAGCUGAAGAAAAACUGUAAGGAAAUAACU |
| | | | GAUAAGGAAUUGGACAAGAAAAUGAAGGAGCUGGCCGCCGUCAUGA |
| | | | GCGACCCUGACCUGGAAACUGAGACUAUGUGCCUCCACGACGACGA |
| | | | GUCGUGUCGCUACGAAGGGCAAGUCGCUGUUUACCAGGAUGUAUAC |
| | | | GCCGUCGACGGCCCCACCAGCCUGUACCACCAGGCCAACAAGGGCG |
| | | | UGAGGGUGGCCUACUGGAUCGGCUUCGACACCACACCCUUCAUGUU |
| | | | CAAGAACCUGGCCGGCGCCUACCCCAGCUACAGCACCAACUGGGCC |
| | | | GACGAGACCGUGCUGACCGCCAGGAACAUCGGCCUGUGCAGCAGCG |
| | | | ACGUGAUGGAGAGGAGCCGGAGAGGCAUGAGCAUCCUGAGGAAGAA |
| | | | AUACCUGAAGCCCAGCAACAACGUGCUGUUCAGCGUGGGCAGCACC |
| | | | AUCUACCACGAGAAGAGGGACCUGCUCAGGAGCUGGCACCUGCCCA |
| | | | GCGUGUUCCACCUGAGGGGCAAGCAGAACUACACCUGCAGGUGCGA |
| | | | GACCAUCGUGAGCUGCGACGGCUACGUGGUGAAGAGGAUCGCCAUC |
| | | | AGCCCCGGCCUGUACGGCAAGCCCAGCGGCUACGCCGCUACAAUGC |
| | | | ACAGGGAGGGCUUCCUGUGCUGCAAGGUGACCGACACCCUGAACGG |
| | | | CGAGAGGGUGAGCUUCCCCGUGUGCACCUACGUGCCCGCCACCCUG |
| | | | UGCGACCAGAUGACCGGCAUCCUGGCCACCGACGUGAGCGCCGACG |
| | | | ACGCCCAGAAGCUGCUCGUGGGCCUGAACCAGAGGAUCGUGGUCAA |
| | | | CGGCAGGACCCAGAGGAACACCAACACAAUGAAGAACUACCUGCUG |
| | | | CCCGUGGUGGCCCAGGCUUUCGCCAGGUGGGCCAAGGAGUACAAGG |
| | | | AGGACCAGGAAGACGAGAGGCCCCUGGGCCUGAGGGACAGGCAGCU |
| | | | GGUGAUGGGCUGCUGCUGGGCCUUCAGGCGGCACAAGAUCACCAGC |
| | | | AUCUACAAGAGGCCCGACACCCAGACCAUCAUCAAGGUGAACAGCG |
| | | | ACUUCCACAGCUUCGUGCUGCCCAGGAUCGGCAGCAACACCCUGGA |
| | | | GAUCGGCCUGAGGACCCGGAUCAGGAAGAUGCUGGAGGAACACAAG |
| | | | GAGCCCAGCCCACUGAUCACCGCCGAGGACGUGCAGGAGGCCAAGU |
| | | | GCGCUGCCGACGAGGCCAAGGAGGUGAGGGAGGCCGAGGAACUGAG |
| | | | GGCCGCCCUGCCACCCCUGGCUGCCGACGUGGAGGAACCCACCCUG |
| | | | GAAGCCGACGUGGACCUGAUGCUGCAGGAGGCCGGCGCCGGAAGCG |
| | | | UGGAGACACCCAGGGGCCUGAUCAAGGUGACCAGCUACGACGGCGA |
| | | | GGACAAGAUCGGCAGCUACGCCGUGCUGAGCCCACAGGCCGUGCUG |
| | | | AAGUCCGAGAAGCUGAGCUGCAUCCACCCACUGGCCGAGCAGGUGA |
| | | | UCGUGAUCACCCACAGCGGCAGGAAGGGCAGGUACGCCGUGGAGCC |
| | | | CUACCACGGCAAGGUGGUCGUGCCCGAGGGCCACGCCAUCCCCGUG |
| | | | CAGGACUUCCAGGCCCUGAGCGAGAGCGCCACCAUCGUGUACAACG |
| | | | AGAGGGAGUUCGUGAACAGGUACCUGCACCAUAUCGCCACCCACGG |
| | | | CGGAGCCCUGAACACCGACGAGGAAUACUACAAGACCGUGAAGCCC |
| | | | AGCGAGCACGACGGCGAGUACCUGUACGACAUCGACAGGAAGCAGU |
| | | | GCGUGAAGAAAGAGCUGGUGACCGGCCUGGGACUGACCGGCGAGCU |
| | | | GGUGGACCCACCCUUCCACGAGUUCGCCUACGAGAGCCUGAGGACC |
| | | | AGACCCGCCGCUCCCUACCAGGUGCCCACCAUCGGCGUGUACGGCG |
| | | | UGCCCGGCAGCGGAAAGAGCGGCAUCAUCAAGAGCGCCGUGACCAA |
| | | | GAAAGACCUGGUGGUCAGCGCCAAGAAAGAGAACUGCGCCGAGAUC |
| | | | AUCAGGGACGUGAAGAAGAUGAAAGGCCUGGACGUGAACGCGCGCA |
| | | | CCGUGGACAGCGUGCUGCUGAACGGCUGCAAGCACCCCGUGGAGAC |
| | | | CCUGUACAUCGACGAGGCCUUCGCUUGCCACGCCGGCACCCUGAGG |
| | | | GCCCUGAUCGCCAUCAUCAGGCCCAAGAAAGCCGUGCUGUGCGGCG |
| | | | ACCCCAAGCAGUGCGGCUUCUUCAACAUGAUGUGCCUGAAGGUGCA |
| | | | CUUCAACCACGAGAUCUGCACCCAGGUGUUCCACAAGAGCAUCAGC |
| | | | AGGCGGUGCACCAAGAGCGUGACCAGCGUCGUGAGCACCCUGUUCU |
| | | | ACGACAAGAAAAUGAGGACCACCAACCCCAAGGAGACCAAAAUCGU |
| | | | GAUCGACACCACAGGCAGCACCAAGCCCAAGCAGGACGACCUGAUC |
| | | | CUGACCUGCUUCAGGGGCUGGGUGAAGCAGCUGCAGAUCGACUACA |
| | | | AGGGCAACGAGAUCAUGACCGCCGCUGCCAGCCAGGGCCUGACCAG |
| | | | GAAGGGCGUGUACGCCGUGAGGUACAAGGUGAACGAGAACCCACUG |
| | | | UACGCUCCCACCAGCGAGCACGUGAACGUGCUGCUGACCAGGACCG |
| | | | AGGACAGGAUCGUGUGGAAGACCCUGGCCGGCGACCCCUGGAUCAA |
| | | | GACCCUGACCGCCAAGUACCCCGGCAACUUCACCGCCACCAUCGAA |
| | | | GAGUGGCAGGCCGAGCACGACGCCAUCAUGAGGCACAUCCUGGAGA |
| | | | GGCCCGACCCCACCGACGUGUUCCAGAACAAGGCCAACGUGUGCUG |
| | | | GGCCAAGGCCCUGGUGCCCGUGCUGAAGACCGCCGGCAUCGACAUG |
| | | | ACCACAGAGCAGUGGAACACCGUGGACUACUUCGAGACCGACAAGG |
| | | | CCCACAGCGCCGAGAUCGUGCUGAACCAGCUGUGCGUGAGGUUCUU |
| | | | CGGCCUGGACCUGGACAGCGGCCUGUUCAGCGCCCCCACCGUGCCA |
| | | | CUGAGCAUCAGGAACAACCACUGGGACAACAGCCCCAGCCCAAACA |
| | | | UGUACGGCCUGAACAAGGAGGUGGUCAGGCAGCUGAGCAGGCGGUA |
| | | | CCCACAGCUGCCCAGGGCCGUGGCCACCGGCAGGGUGUACGACAUG |
| | | | AACACCGGCACCCUGAGGAACUACGACCCCAGGAUCAACCUGGUGC |
| | | | CCGUGAACAGGCGGCUGCCCCACGCCCUGGUGCUGCACCACAACGA |
| | | | GCACCCACAGAGCGACUUCAGCUCCUUCGUGAGCAAGCUGAAAGGC |
| | | | AGGACCGUGCUGGUCGUGGGCGAGAAGCUGAGCGUGCCCGGCAAGA |
| | | | UGGUGGACUGGCUGAGCGACAGGCCCGAGGCCACCUUCCGGGCCAG |
| | | | GCUGGACCUCGGCAUCCCCGGCGACGUGCCCAAGUACGACAUCAUC |
| | | | UUCGUGAACGUCAGGACCCCAUACAAGUACCACCAUUACCAGCAGU |
| | | | GCGAGGACCACGCCAUCAAGCUGAGCAUGCUGACCAAGAAGGCCUG |
| | | | CCUGCACCUGAACCCCGGAGGCACCUGCGUGAGCAUCGGCUACGGC |
| | | | UACGCCGACAGGGCCAGCGAGAGCAUCAUUGGCGCCAUCGCCAGGC |
| | | | UGUUCAAGUUCAGCAGGGUGUGCAAACCCAAGAGCAGCCUGGAGGA |
| | | | AACCGAGGUGCUGUUCGUGUUCAUCGGCUACGACCGGAAGGCCAGG |
| | | | ACCCACAACCCCUACAAGCUGAGCAGCACCCUGACAAACAUCUACA |
| | | | CCGGCAGCAGGCUGCACGAGGCCGGCUGCGCCCCCAGCUACCACGU |
| | | | GGUCAGGGGCGAUAUCGCCACCGCCACCGAGGGCGUGAUCAUCAAC |
| | | | GCUGCCAACAGCAAGGGCCAGCCCGGAGGCGGAGUGUGCGGCGCCC |
| | | | UGUACAAGAAGUUCCCCGAGAGCUUCGACCUGCAGCCCAUCGAGGU |
| | | | GGGCAAGGCCAGGCUGGUGAAGGGCGCCGCUAAGCACAUCAUCCAC |
| | | | GCCGUGGGCCCCAACUUCAACAAGGUGAGCGAGGUGGAAGGCGACA |
| | | | AGCAGCUGGCCGAAGCCUACGAGAGCAUCGCCAAGAUCGUGAACGA |
| | | | CAAUAACUACAAGAGCGUGGCCAUCCCACUGCUCAGCACCGGCAUC |
| | | | UUCAGCGGCAACAAGGACAGGCUGACCCAGAGCCUGAACCACCUGC |
| | | | UCACCGCCCUGGACACCACCGAUGCCGACGUGGCCAUCUACUGCAG |
| | | | GGACAAGAAGUGGGAGAUGACCCUGAAGGAGGCCGUGGCCAGGCGG |
| | | | GAGGCCGUGGAAGAGAUCUGCAUCAGCGACGACUCCAGCGUGACCG |
| | | | AGCCCGACGCCGAGCUGGUGAGGGUGCACCCCAAGAGCUCCCUGGC |
| | | | CGGCAGGAAGGGCUACAGCACCAGCGACGGCAAGACCUUCAGCUAC |
| | | | CUGGAGGGCACCAAGUUCCACCAGGCCGCUAAGGACAUCGCCGAGA |
| | | | UCAACGCUAUGUGGCCCGUGGCCACCGAGGCCAACGAGCAGGUGUG |
| | | | CAUGUACAUCCUGGGCGAGAGCAUGUCCAGCAUCAGGAGCAAGUGC |
| | | | CCCGUGGAGGAAAGCGAGGCCAGCACACCACCCAGCACCCUGCCCU |
| | | | GCCUGUGCAUCCACGCUAUGACACCCGAGAGGGUGCAGCGGCUGAA |
| | | | GGCCAGCAGGCCCGAGCAGAUCACCGUGUGCAGCUCCUUCCCACUG |
| | | | CCCAAGUACAGGAUCACCGGCGUGCAGAAGAUCCAGUGCAGCCAGC |
| | | | CCAUCCUGUUCAGCCCAAAGGUGCCCGCCUACAUCCACCCCAGGAA |
| | | | GUACCUGGUGGAGACCCCACCCGUGGACGAGACACCCGAGCCAAGC |
| | | | GCCGAGAACCAGAGCACCGAGGGCACACCCGAGCAGCCACCCCUGA |
| | | | UCACCGAGGACGAGACAAGGACCCGGACCCCAGAGCCCAUCAUUAU |
| | | | CGAGGAAGAGGAAGAGGACAGCAUCAGCCUGCUGAGCGACGGCCCC |
| | | | ACCCACCAGGUGCUGCAGGUGGAGGCCGACAUCCACGGCCCACCCA |
| | | | GCGUGUCCAGCUCCAGCUGGAGCAUCCCACACGCCAGCGACUUCGA |
| | | | CGUGGACAGCCUGAGCAUCCUGGACACCCUGGAGGGCGCCAGCGUG |
| | | | ACCUCCGGCGCCACCAGCGCCGAGACCAACAGCUACUUCGCCAAGA |
| | | | GCAUGGAGUUCCUGGCCAGGCCCGUGCCAGCUCCCAGGACCGUGUU |
| | | | CAGGAACCCACCCCACCCAGCUCCCAGGACCAGGACCCCAAGCCUG |
| | | | GCUCCCAGCAGGGCCUGCAGCAGGACCAGCCUGGUGAGCACCCCAC |
| | | | CCGGCGUGAACAGGGUGAUCACCAGGGAGGAACUGGAGGCCCUGAC |
| | | | ACCCAGCAGGACCCCCAGCAGGUCCGUGAGCAGGACUAGUCUGGUG |
| | | | UCCAACCCACCCGGCGUGAACAGGGUGAUCACCAGGGAGGAAUUCG |
| | | | AGGCCUUCGUGGCCCAGCAACAGAGACGGUUCGACGCCGGCGCCUA |
| | | | CAUCUUCAGCAGCGACACCGGCCAGGGACACCUGCAGCAAAAGAGC |
| | | | GUGAGGCAGACCGUGCUGAGCGAGGUGGUGCUGGAGAGGACCGAGC |
| | | | UGGAAAUCAGCUACGCCCCCAGGCUGGACCAGGAGAAGGAGGAACU |
| | | | GCUCAGGAAGAAACUGCAGCUGAACCCCACCCCAGCCAACAGGAGC |
| | | | AGGUACCAGAGCAGGAAGGUGGAGAACAUGAAGGCCAUCACCGCCA |
| | | | GGCGGAUCCUGCAGGGCCUGGGACACUACCUGAAGGCCGAGGGCAA |
| | | | GGUGGAGUGCUACAGGACCCUGCACCCCGUGCCACUGUACAGCUCC |
| | | | AGCGUGAACAGGGCCUUCUCCAGCCCCAAGGUGGCCGUGGAGGCCU |
| | | | GCAACGCUAUGCUGAAGGAGAACUUCCCCACCGUGGCCAGCUACUG |
| | | | CAUCAUCCCCGAGUACGACGCCUACCUGGACAUGGUGGACGGCGCC |
| | | | AGCUGCUGCCUGGACACCGCCAGCUUCUGCCCCGCCAAGCUGAGGA |
| | | | GCUUCCCCAAGAAACACAGCUACCUGGAGCCCACCAUCAGGAGCGC |
| | | | CGUGCCCAGCGCCAUCCAGAACACCCUGCAGAACGUGCUGGCCGCU |
| | | | GCCACCAAGAGGAACUGCAACGUGACCCAGAUGAGGGAGCUGCCCG |
| | | | UGCUGGACAGCGCUGCCUUCAACGUGGAGUGCUUCAAGAAAUACGC |
| | | | CUGCAACAACGAGUACUGGGAGACCUUCAAGGAGAACCCCAUCAGG |
| | | | CUGACCGAAGAGAACGUGGUGAACUACAUCACCAAGCUGAAGGGCC |
| | | | CCAAGGCCGCUGCCCUGUUCGCUAAGACCCACAACCUGAACAUGCU |
| | | | GCAGGACAUCCCAAUGGACAGGUUCGUGAUGGACCUGAAGAGGGAC |
| | | | GUGAAGGUGACACCCGGCACCAAGCACACCGAGGAGAGGCCCAAGG |
| | | | UGCAGGUGAUCCAGGCCGCUGACCCACUGGCCACCGCCUACCUGUG |
| | | | CGGCAUCCACAGGGAGCUGGUGAGGCGGCUGAACGCCGUGCUGCUG |
| | | | CCCAACAUCCACACCCUGUUCGACAUGAGCGCCGAGGACUUCGACG |
| | | | CCAUCAUCGCCGAGCACUUCCAGCCCGGCGACUGCGUGCUGGAGAC |
| | | | CGACAUCGCCAGCUUCGACAAGAGCGAGGAUGACGCUAUGGCCCUG |
| | | | ACCGCUCUGAUGAUCCUGGAGGACCUGGGCGUGGACGCCGAGCUGC |
| | | | UCACCCUGAUCGAGGCUGCCUUCGGCGAGAUCAGCUCCAUCCACCU |
| | | | GCCCACCAAGACCAAGUUCAAGUUCGGCGCUAUGAUGAAAAGCGGA |
| | | | AUGUUCCUGACCCUGUUCGUGAACACCGUGAUCAACAUUGUGAUCG |
| | | | CCAGCAGGGUGCUGCGGGAGAGGCUGACCGGCAGCCCCUGCGCUGC |
| | | | CUUCAUCGGCGACGACAACAUCGUGAAGGGCGUGAAAAGCGACAAG |
| | | | CUGAUGGCCGACAGGUGCGCCACCUGGCUGAACAUGGAGGUGAAGA |
| | | | UCAUCGACGCCGUGGUGGGCGAGAAGGCCCCCUACUUCUGCGGCGG |
| | | | AUUCAUCCUGUGCGACAGCGUGACCGGCACCGCCUGCAGGGUGGCC |
| | | | GACCCCCUGAAGAGGCUGUUCAAGCUGGGCAAGCCACUGGCCGCUG |
| | | | ACGAUGAGCACGACGAUGACAGGCGGAGGGCCCUGCACGAGGAAAG |
| | | | CACCAGGUGGAACAGGGUGGGCAUCCUGAGCGAGCUGUGCAAGGCC |
| | | | GUGGAGAGCAGGUACGAGACCGUGGGCACCAGCAUCAUCGUGAUGG |
| | | | CUAUGACCACACUGGCCAGCUCCGUCAAGAGCUUCUCCUACCUGAG |
| | | | GGGGGCCCCUAUAACUCUCUACGGCUAACCUGAAUGGACUACGACA |
| | | | UAGUCUAGUCCGCCAAGGCCGCCACCAUGGAAGAUGCCAAAAACAU |
| | | | UAAGAAGGGCCCAGCGCCAUUCUACCCACUCGAAGACGGGACCGCC |
| | | | GGCGAGCAGCUGCACAAAGCCAUGAAGCGCUACGCCCUGGUGCCCG |
| | | | GCACCAUCGCCUUUACCGACGCACAUAUCGAGGUGGACAUUACCUA |
| | | | CGCCGAGUACUUCGAGAUGAGCGUUCGGCUGGCAGAAGCUAUGAAG |
| | | | CGCUAUGGGCUGAAUACAAACCAUCGGAUCGUGGUGUGCAGCGAGA |
| | | | AUAGCUUGCAGUUCUUCAUGCCCGUGUUGGGUGCCCUGUUCAUCGG |
| | | | UGUGGCUGUGGCCCCAGCUAACGACAUCUACAACGAGCGCGAGCUG |
| | | | CUGAACAGCAUGGGCAUCAGCCAGCCCACCGUCGUAUUCGUGAGCA |
| | | | AGAAAGGGCUGCAAAAGAUCCUCAACGUGCAAAAGAAGCUACCGAU |
| | | | CAUACAAAAGAUCAUCAUCAUGGAUAGCAAGACCGACUACCAGGGC |
| | | | UUCCAAAGCAUGUACACCUUCGUGACUUCCCAUUUGCCACCCGGCU |
| | | | UCAACGAGUACGACUUCGUGCCCGAGAGCUUCGACCGGGACAAAAC |
| | | | CAUCGCCCUGAUCAUGAACAGUAGUGGCAGUACCGGAUUGCCCAAG |
| | | | GGCGUAGCCCUACCGCACCGCACCGCUUGUGUCCGAUUCAGUCAUG |
| | | | CCCGCGACCCCAUCUUCGGCAACCAGAUCAUCCCCGACACCGCUAU |
| | | | CCUCAGCGUGGUGCCAUUUCACCACGGCUUCGGCAUGUUCACCACG |
| | | | CUGGGCUACUUGAUCUGCGGCUUUCGGGUCGUGCUCAUGUACCGCU |
| | | | UCGAGGAGGAGCUAUUCUUGCGCAGCUUGCAAGACUAUAAGAUUCA |
| | | | AUCUGCCCUGCUGGUGCCCACACUAUUUAGCUUCUUCGCUAAGAGC |
| | | | ACUCUCAUCGACAAGUACGACCUAAGCAACUUGCACGAGAUCGCCA |
| | | | GCGGCGGGGCGCCGCUCAGCAAGGAGGUAGGUGAGGCCGUGGCCAA |
| | | | ACGCUUCCACCUACCAGGCAUCCGACAGGGCUACGGCCUGACAGAA |
| | | | ACAACCAGCGCCAUUCUGAUCACCCCCGAAGGGGACGACAAGCCUG |
| | | | GCGCAGUAGGCAAGGUGGUGCCCUUCUUCGAGGCUAAGGUGGUGGA |
| | | | CUUGGACACCGGUAAGACACUGGGUGUGAACCAGCGCGGCGAGCUG |
| | | | UGCGUCCGUGGCCCCAUGAUCAUGAGCGGCUACGUUAACAACCCCG |
| | | | AGGCUACAAACGCUCUCAUCGACAAGGACGGCUGGCUGCACAGCGG |
| | | | CGACAUCGCCUACUGGGACGAGGACGAGCACUUCUUCAUCGUGGAC |
| | | | CGGCUGAAGUCCCUGAUCAAAUACAAGGGCUACCAGGUAGCCCCAG |
| | | | CCGAACUGGAGAGCAUCCUGCUGCAACACCCCAACAUCUUCGACGC |
| | | | CGGGGUCGCCGGCCUGCCCGACGACGAUGCCGGCGAGCUGCCCGCC |
| | | | GCAGUCGUCGUGCUGGAACACGGUAAAACCAUGACCGAGAAGGAGA |
| | | | UCGUGGACUAUGUGGCCAGCCAGGUUACAACCGCCAAGAAGCUGCG |
| | | | CGGUGGUGUUGUGUUCGUGGACGAGGUGCCUAAAGGACUGACCGGC |
| | | | AAGUUGGACGCCCGCAAGAUCCGCGAGAUUCUCAUUAAGGCCAAGA |
| | | | AGGGCGGCAAGAUCGCCGUGUAACUCGAGUAUGUUACGUGCAAAGG |
| | | | UGAUUGUCACCCCCCGAAAGACCAUAUUGUGACACACCCUCAGUAU |
| | | | CACGCCCAAACAUUUACAGCCGCGGUGUCAAAAACCGCGUGGACGU |
| | | | GGUUAACAUCCCUGCUGGGAGGAUCAGCCGUAAUUAUUAUAAUUGG |
| | | | CUUGGUGCUGGCUACUAUUGUGGCCAUGUACGUGCUGACCAACCAG |
| | | | AAACAUAAUUGAAUACAGCAGCAAUUGGCAAGCUGCUUACAUAGAA |
| | | | CUCGCGGCGAUUGGCAUGCCGCCUUAAAAUUUUUAUUUUAUUUUUU |
| | | | CUUUUCUUUUCCGAAUCGGAUUUUGUUUUUAAUAUUUCAAAAAAAA |
| | | | AAAAAAAAAAAAAAAAAUCUAGAAAAAAAAAAAAAAAAAAAAAAAA |
| | | | AAAAAAAAAAAAAAAAAAAAAAAAAAAAAAAAAAAAAAAAAAAAAA |
| | | | AAAAAAAAAAAAAAAAAAAAAAAAAAAAAA |
| 2842 | SINV | 2842 | AUUGACGGCGUAGUACACACUAUUGAAUCAAACAGCCGACCAAUUG |
| (SEQ | Fluc | | CACUACCAUCACAAUGGAGAAGCCAGUAGUAAACGUAGACGUAGAC |
| ID | | | CCCCAGAGUCCGUUUGUCGUGCAACUGCAAAAAAGCUUCCCGCAAU |
| NO: | | | UUGAGGUAGUAGCACAGCAGGUCACUCCAAAUGACCAUGCUAAUGC |
| 101) | | | CAGAGCAUUUUCGCAUCUGGCCAGUAAACUAAUCGAGCUGGAGGUU |
| | | | CCUACCACAGCGACGAUCUUGGACAUAGGCAGCGCACCGGCUCGUA |
| | | | GAAUGUUUUCCGAGCACCAGUAUCAUUGUGUCUGCCCCAUGCGUAG |
| | | | UCCAGAAGACCCGGACCGCAUGAUGAAAUAUGCCAGUAAACUGGCG |
| | | | GAAAAAGCGUGCAAGAUUACAAACAAGAACUUGCAUGAGAAGAUUA |
| | | | AGGAUCUCCGGACCGUACUUGAUACGCCGGAUGCUGAAACACCAUC |
| | | | GCUCUGCUUUCACAACGAUGUUACCUGCAACAUGCGUGCCGAAUAU |
| | | | UCCGUCAUGCAGGACGUGUAUAUCAACGCUCCCGGAACUAUCUAUC |
| | | | AUCAGGCUAUGAAAGGCGUGCGGACCCUGUACUGGAUUGGCUUCGA |
| | | | CACCACCCAGUUCAUGUUCUCGGCUAUGGCAGGUUCGUACCCUGCG |
| | | | UACAACACCAACUGGGCCGACGAGAAAGUCCUUGAAGCGCGUAACA |
| | | | UCGGACUUUGCAGCACAAAGCUGAGUGAAGGUAGGACAGGAAAAUU |
| | | | GUCGAUAAUGAGGAAGAAGGAGUUGAAGCCCGGGUCGCGGGUUUAU |
| | | | UUCUCCGUAGGAUCGACACUUUAUCCAGAACACAGAGCCAGCUUGC |
| | | | AGAGCUGGCAUCUUCCAUCGGUGUUCCACUUGAAUGGAAAGCAGUC |
| | | | GUACACUUGCCGCUGUGAUACAGUGGUGAGUUGCGAAGGCUACGUA |
| | | | GUGAAGAAAAUCACCAUCAGUCCCGGGAUCACGGGAGAAACCGUGG |
| | | | GAUACGCGGUUACACACAAUAGCGAGGGCUUCUUGCUAUGCAAAGU |
| | | | UACUGACACAGUAAAAGGAGAACGGGUAUCGUUCCCUGUGUGCACG |
| | | | UACAUCCCGGCCACCAUAUGCGAUCAGAUGACUGGUAUAAUGGCCA |
| | | | CGGAUAUAUCACCUGACGAUGCACAAAAACUUCUGGUUGGGCUCAA |
| | | | CCAGCGAAUUGUCAUUAACGGUAGGACUAACAGGAACACCAACACC |
| | | | AUGCAAAAUUACCUUCUGCCGAUCAUAGCACAAGGGUUCAGCAAAU |
| | | | GGGCUAAGGAGCGCAAGGAUGAUCUUGAUAACGAGAAAAUGCUGGG |
| | | | UACUAGAGAACGCAAGCUUACGUAUGGCUGCUUGUGGGCGUUUCGC |
| | | | ACUAAGAAAGUACAUUCGUUUUAUCGCCCACCUGGAACGCAGACCU |
| | | | GCGUAAAAGUCCCAGCCUCUUUUAGCGCUUUUCCCAUGUCGUCCGU |
| | | | AUGGACGACCUCUUUGCCCAUGUCGCUGAGGCAGAAAUUGAAACUG |
| | | | GCAUUGCAACCAAAGAAGGAGGAAAAACUGCUGCAGGUCUCGGAGG |
| | | | AAUUAGUCAUGGAGGCCAAGGCUGCUUUUGAGGAUGCUCAGGAGGA |
| | | | AGCCAGAGCGGAGAAGCUCCGAGAAGCACUUCCACCAUUAGUGGCA |
| | | | GACAAAGGCAUCGAGGCAGCCGCAGAAGUUGUCUGCGAAGUGGAGG |
| | | | GGCUCCAGGCGGACAUCGGAGCAGCAUUAGUUGAAACCCCGCGCGG |
| | | | UCACGUAAGGAUAAUACCUCAAGCAAAUGACCGUAUGAUCGGACAG |
| | | | UAUAUCGUUGUCUCGCCAAACUCUGUGCUGAAGAAUGCCAAACUCG |
| | | | CACCAGCGCACCCGCUAGCAGAUCAGGUUAAGAUCAUAACACACUC |
| | | | CGGAAGAUCAGGAAGGUACGCGGUCGAACCAUACGACGCUAAAGUA |
| | | | CUGAUGCCAGCAGGAGGUGCCGUACCAUGGCCAGAAUUCCUAGCAC |
| | | | UGAGUGAGAGCGCCACGUUAGUGUACAACGAAAGAGAGUUUGUGAA |
| | | | CCGCAAACUAUACCACAUUGCCAUGCAUGGCCCCGCCAAGAAUACA |
| | | | GAAGAGGAGCAGUACAAGGUUACAAAGGCAGAGCUUGCAGAAACAG |
| | | | AGUACGUGUUUGACGUGGACAAGAAGCGUUGCGUUAAGAAGGAAGA |
| | | | AGCCUCAGGUCUGGUCCUCUCGGGAGAACUGACCAACCCUCCCUAU |
| | | | CAUGAGCUAGCUCUGGAGGGACUGAAGACCCGACCUGCGGUCCCGU |
| | | | ACAAGGUCGAAACAAUAGGAGUGAUAGGCACACCGGGGUCGGGCAA |
| | | | GUCAGCUAUUAUCAAGUCAACUGUCACGGCACGAGAUCUUGUUACC |
| | | | AGCGGAAAGAAAGAAAAUUGUCGCGAAAUUGAGGCCGACGUGCUAA |
| | | | GACUGAGGGGUAUGCAGAUUACGUCGAAGACAGUAGAUUCGGUUAU |
| | | | GCUCAACGGAUGCCACAAAGCCGUAGAAGUGCUGUACGUUGACGAA |
| | | | GCGUUCGCGUGCCACGCAGGAGCACUACUUGCCUUGAUUGCUAUCG |
| | | | UCAGGCCCCGCAAGAAGGUAGUACUAUGCGGAGACCCCAUGCAAUG |
| | | | CGGAUUCUUCAACAUGAUGCAACUAAAGGUACAUUUCAAUCACCCU |
| | | | GAAAAAGACAUAUGCACCAAGACAUUCUACAAGUAUAUCUCCCGGC |
| | | | GUUGCACACAGCCAGUUACAGCUAUUGUAUCGACACUGCAUUACGA |
| | | | UGGAAAGAUGAAAACCACGAACCCGUGCAAGAAGAACAUUGAAAUC |
| | | | GAUAUUACAGGGGCCACAAAGCCGAAGCCAGGGGAUAUCAUCCUGA |
| | | | CAUGUUUCCGCGGGUGGGUUAAGCAAUUGCAAAUCGACUAUCCCGG |
| | | | ACAUGAAGUAAUGACAGCCGCGGCCUCACAAGGGCUAACCAGAAAA |
| | | | GGAGUGUAUGCCGUCCGGCAAAAAGUCAAUGAAAACCCACUGUACG |
| | | | CGAUCACAUCAGAGCAUGUGAACGUGUUGCUCACCCGCACUGAGGA |
| | | | CAGGCUAGUGUGGAAAACCUUGCAGGGCGACCCAUGGAUUAAGCAG |
| | | | CUCACUAACAUACCUAAAGGAAACUUUCAGGCUACUAUAGAGGACU |
| | | | GGGAAGCUGAACACAAGGGAAUAAUUGCUGCAAUAAACAGCCCCAC |
| | | | UCCCCGUGCCAAUCCGUUCAGCUGCAAGACCAACGUUUGCUGGGCG |
| | | | AAAGCAUUGGAACCGAUACUAGCCACGGCCGGUAUCGUACUUACCG |
| | | | GUUGCCAGUGGAGCGAACUGUUCCCACAGUUUGCGGAUGACAAACC |
| | | | ACAUUCGGCCAUUUACGCCUUAGACGUAAUUUGCAUUAAGUUUUUC |
| | | | GGCAUGGACUUGACAAGCGGACUGUUUUCUAAACAGAGCAUCCCAC |
| | | | UAACGUACCAUCCCGCCGAUUCAGCGAGGCCGGUAGCUCAUUGGGA |
| | | | CAACAGCCCAGGAACCCGCAAGUAUGGGUACGAUCACGCCAUUGCC |
| | | | GCCGAACUCUCCCGUAGAUUUCCGGUGUUCCAGCUAGCUGGGAAGG |
| | | | GCACACAACUUGAUUUGCAGACGGGGAGAACCAGAGUUAUCUCUGC |
| | | | ACAGCAUAACCUGGUCCCGGUGAACCGCAAUCUUCCUCACGCCUUA |
| | | | GUCCCCGAGUACAAGGAGAAGCAACCCGGCCCGGUCGAAAAAUUCU |
| | | | UGAACCAGUUCAAACACCACUCAGUACUUGUGGUAUCAGAGGAAAA |
| | | | AAUUGAAGCUCCCCGUAAGAGAAUCGAAUGGAUCGCCCCGAUUGGC |
| | | | AUAGCCGGUGCAGAUAAGAACUACAACCUGGCUUUCGGGUUUCCGC |
| | | | CGCAGGCACGGUACGACCUGGUGUUCAUCAACAUUGGAACUAAAUA |
| | | | CAGAAACCACCACUUUCAGCAGUGCGAAGACCAUGCGGCGACCUUA |
| | | | AAAACCCUUUCGCGUUCGGCCCUGAAUUGCCUUAACCCAGGAGGCA |
| | | | CCCUCGUGGUGAAGUCCUAUGGCUACGCCGACCGCAACAGUGAGGA |
| | | | CGUAGUCACCGCUCUUGCCAGAAAGUUUGUCAGGGUGUCUGCAGCG |
| | | | AGACCAGAUUGUGUCUCAAGCAAUACAGAAAUGUACCUGAUUUUCC |
| | | | GACAACUAGACAACAGCCGUACACGGCAAUUCACCCCGCACCAUCU |
| | | | GAAUUGCGUGAUUUCGUCCGUGUAUGAGGGUACAAGAGAUGGAGUU |
| | | | GGAGCCGCGCCGUCAUACCGCACCAAAAGGGAGAAUAUUGCUGACU |
| | | | GUCAAGAGGAAGCAGUUGUCAACGCAGCCAAUCCGCUGGGUAGACC |
| | | | AGGCGAAGGAGUCUGCCGUGCCAUCUAUAAACGUUGGCCGACCAGU |
| | | | UUUACCGAUUCAGCCACGGAGACAGGCACCGCAAGAAUGACUGUGU |
| | | | GCCUAGGAAAGAAAGUGAUCCACGCGGUCGGCCCUGAUUUCCGGAA |
| | | | GCACCCAGAAGCAGAAGCCUUGAAAUUGCUACAAAACGCCUACCAU |
| | | | GCAGUGGCAGACUUAGUAAAUGAACAUAACAUCAAGUCUGUCGCCA |
| | | | UUCCACUGCUAUCUACAGGCAUUUACGCAGCCGGAAAAGACCGCCU |
| | | | UGAAGUAUCACUUAACUGCUUGACAACCGCGCUAGACAGAACUGAC |
| | | | GCGGACGUAACCAUCUAUUGCCUGGAUAAGAAGUGGAAGGAAAGAA |
| | | | UCGACGCGGCACUCCAACUUAAGGAGUCUGUAACAGAGCUGAAGGA |
| | | | UGAAGAUAUGGAGAUCGACGAUGAGUUAGUAUGGAUCCAUCCAGAC |
| | | | AGUUGCUUGAAGGGAAGAAAGGGAUUCAGUACUACAAAAGGAAAAU |
| | | | UGUAUUCGUACUUCGAAGGCACCAAAUUCCAUCAAGCAGCAAAAGA |
| | | | CAUGGCGGAGAUAAAGGUCCUGUUCCCUAAUGACCAGGAAAGUAAU |
| | | | GAACAACUGUGUGCCUACAUAUUGGGUGAGACCAUGGAAGCAAUCC |
| | | | GCGAAAAGUGCCCGGUCGACCAUAACCCGUCGUCUAGCCCGCCCAA |
| | | | AACGUUGCCGUGCCUUUGCAUGUAUGCCAUGACGCCAGAAAGGGUC |
| | | | CACAGACUUAGAAGCAAUAACGUCAAAGAAGUUACAGUAUGCUCCU |
| | | | CCACCCCCCUUCCUAAGCACAAAAUUAAGAAUGUUCAGAAGGUUCA |
| | | | GUGCACGAAAGUAGUCCUGUUUAAUCCGCACACUCCCGCAUUCGUU |
| | | | CCCGCCCGUAAGUACAUAGAAGUGCCAGAACAGCCUACCGCUCCUC |
| | | | CUGCACAGGCCGAGGAGGCCCCCGAAGUUGUAGCGACACCGUCACC |
| | | | AUCUACAGCUGAUAACACCUCGCUUGAUGUCACAGACAUCUCACUG |
| | | | GAUAUGGAUGACAGUAGCGAAGGCUCACUUUUUUCGAGCUUUAGCG |
| | | | GAUCGGACAACUCUAUUACUAGUAUGGACAGUUGGUCGUCAGGACC |
| | | | UAGUUCACUAGAGAUAGUAGACCGAAGGCAGGUGGUGGUGGCUGAC |
| | | | GUUCAUGCCGUCCAAGAGCCUGCCCCUAUUCCACCGCCAAGGCUAA |
| | | | AGAAGAUGGCCCGCCUGGCAGCGGCAAGAAAAGAGCCCACUCCACC |
| | | | GGCAAGCAAUAGCUCUGAGUCCCUCCACCUCUCUUUUGGUGGGGUA |
| | | | UCCAUGUCCCUCGGAUCAAUUUUCGACGGAGAGACGGCCCGCCAGG |
| | | | CAGCGGUACAACCCCUGGCAACAGGCCCCACGGAUGUGCCUAUGUC |
| | | | UUUCGGAUCGUUUUCCGACGGAGAGAUUGAUGAGCUGAGCCGCAGA |
| | | | GUAACUGAGUCCGAACCCGUCCUGUUUGGAUCAUUUGAACCGGGCG |
| | | | AAGUGAACUCAAUUAUAUCGUCCCGAUCAGCCGUAUCUUUUCCUCU |
| | | | ACGCAAGCAGAGACGUAGACGCAGGAGCAGGAGGACUGAAUACUGA |
| | | | CUAACCGGGGUAGGUGGGUACAUAUUUUCGACGGACACAGGCCCUG |
| | | | GGCACUUGCAAAAGAAGUCCGUUCUGCAGAACCAGCUUACAGAACC |
| | | | GACCUUGGAGCGCAAUGUCCUGGAAAGAAUUCAUGCCCCGGUGCUC |
| | | | GACACGUCGAAAGAGGAACAACUCAAACUCAGGUACCAGAUGAUGC |
| | | | CCACCGAAGCCAACAAAAGUAGGUACCAGUCUCGUAAAGUAGAAAA |
| | | | UCAGAAAGCCAUAACCACUGAGCGACUACUGUCAGGACUACGACUG |
| | | | UAUAACUCUGCCACAGAUCAGCCAGAAUGCUAUAAGAUCACCUAUC |
| | | | CGAAACCAUUGUACUCCAGUAGCGUACCGGCGAACUACUCCGAUCC |
| | | | ACAGUUCGCUGUAGCUGUCUGUAACAACUAUCUGCAUGAGAACUAU |
| | | | CCGACAGUAGCAUCUUAUCAGAUUACUGACGAGUACGAUGCUUACU |
| | | | UGGAUAUGGUAGACGGGACAGUCGCCUGCCUGGACACUGCAACCUU |
| | | | CUGCCCCGCUAAGCUUAGAAGUUACCCGAAAAAACAUGAGUAUAGA |
| | | | GCCCCGAAUAUCCGCAGUGCGGUUCCAUCAGCGAUGCAGAACACGC |
| | | | UACAAAAUGUGCUCAUUGCCGCAACUAAAAGAAAUUGCAACGUCAC |
| | | | GCAGAUGCGUGAACUGCCAACACUGGACUCAGCGACAUUCAAUGUC |
| | | | GAAUGCUUUCGAAAAUAUGCAUGUAAUGACGAGUAUUGGGAGGAGU |
| | | | UCGCUCGGAAGCCAAUUAGGAUUACCACUGAGUUUGUCACCGCAUA |
| | | | UGUAGCUAGACUGAAAGGCCCUAAGGCCGCCGCACUAUUUGCAAAG |
| | | | ACGUAUAAUUUGGUCCCAUUGCAAGAAGUGCCUAUGGAUAGAUUCG |
| | | | UCAUGGACAUGAAAAGAGACGUGAAAGUUACACCAGGCACGAAACA |
| | | | CACAGAAGAAAGACCGAAAGUACAAGUGAUACAAGCCGCAGAACCC |
| | | | CUGGCGACUGCUUACUUAUGCGGGAUUCACCGGGAAUUAGUGCGUA |
| | | | GGCUUACGGCCGUCUUGCUUCCAAACAUUCACACGCUUUUUGACAU |
| | | | GUCGGCGGAGGAUUUUGAUGCAAUCAUAGCAGAACACUUCAAGCAA |
| | | | GGCGACCCGGUACUGGAGACGGAUAUCGCAUCAUUCGACAAAAGCC |
| | | | AAGACGACGCUAUGGCGUUAACCGGUCUGAUGAUCUUGGAGGACCU |
| | | | GGGUGUGGAUCAACCACUACUCGACUUGAUCGAGUGCGCCUUUGGA |
| | | | GAAAUAUCAUCCACCCAUCUACCUACGGGUACUCGUUUUAAAUUCG |
| | | | GGGCGAUGAUGAAAUCCGGAAUGUUCCUCACACUUUUUGUCAACAC |
| | | | AGUUUUGAAUGUCGUUAUCGCCAGCAGAGUACUAGAGGAGCGGCUU |
| | | | AAAACGUCCAGAUGUGCAGCGUUCAUUGGCGACGACAACAUCAUAC |
| | | | AUGGAGUAGUAUCUGACAAAGAAAUGGCUGAGAGGUGCGCCACCUG |
| | | | GCUCAACAUGGAGGUUAAGAUCAUCGACGCAGUCAUCGGUGAGAGA |
| | | | CCACCUUACUUCUGCGGCGGAUUUAUCUUGCAAGAUUCGGUUACUU |
| | | | CCACAGCGUGCCGCGUGGCGGAUCCCCUGAAAAGGCUGUUUAAGUU |
| | | | GGGUAAACCGCUCCCAGCCGACGACGAGCAAGACGAAGACAGAAGA |
| | | | CGCGCUCUGCUAGAUGAAACAAAGGCGUGGUUUAGAGUAGGUAUAA |
| | | | CAGGCACUUUAGCAGUGGCCGUGACGACCCGGUAUGAGGUAGACAA |
| | | | UAUUACACCUGUCCUACUGGCAUUGAGAACUUUUGCCCAGAGCAAA |
| | | | AGAGCAUUCCAAGCCAUCAGAGGGGAAAUAAAGCAUCUCUACGGUG |
| | | | GUCCUAAAUAGUCAGCAUAGUACAUUUCAUCUGACUAAUACUACAA |
| | | | CACCACCACCAUGGAAGAUGCCAAAAACAUUAAGAAGGGCCCAGCG |
| | | | CCAUUCUACCCACUCGAAGACGGGACCGCCGGCGAGCAGCUGCACA |
| | | | AAGCCAUGAAGCGCUACGCCCUGGUGCCCGGCACCAUCGCCUUUAC |
| | | | CGACGCACAUAUCGAGGUGGACAUUACCUACGCCGAGUACUUCGAG |
| | | | AUGAGCGUUCGGCUGGCAGAAGCUAUGAAGCGCUAUGGGCUGAAUA |
| | | | CAAACCAUCGGAUCGUGGUGUGCAGCGAGAAUAGCUUGCAGUUCUU |
| | | | CAUGCCCGUGUUGGGUGCCCUGUUCAUCGGUGUGGCUGUGGCCCCA |
| | | | GCUAACGACAUCUACAACGAGCGCGAGCUGCUGAACAGCAUGGGCA |
| | | | UCAGCCAGCCCACCGUCGUAUUCGUGAGCAAGAAAGGGCUGCAAAA |
| | | | GAUCCUCAACGUGCAAAAGAAGCUACCGAUCAUACAAAAGAUCAUC |
| | | | AUCAUGGAUAGCAAGACCGACUACCAGGGCUUCCAAAGCAUGUACA |
| | | | CCUUCGUGACUUCCCAUUUGCCACCCGGCUUCAACGAGUACGACUU |
| | | | CGUGCCCGAGAGCUUCGACCGGGACAAAACCAUCGCCCUGAUCAUG |
| | | | AACAGUAGUGGCAGUACCGGAUUGCCCAAGGGCGUAGCCCUACCGC |
| | | | ACCGCACCGCUUGUGUCCGAUUCAGUCAUGCCCGCGACCCCAUCUU |
| | | | CGGCAACCAGAUCAUCCCCGACACCGCUAUCCUCAGCGUGGUGCCA |
| | | | UUUCACCACGGCUUCGGCAUGUUCACCACGCUGGGCUACUUGAUCU |
| | | | GCGGCUUUCGGGUCGUGCUCAUGUACCGCUUCGAGGAGGAGCUAUU |
| | | | CUUGCGCAGCUUGCAAGACUAUAAGAUUCAAUCUGCCCUGCUGGUG |
| | | | CCCACACUAUUUAGCUUCUUCGCUAAGAGCACUCUCAUCGACAAGU |
| | | | ACGACCUAAGCAACUUGCACGAGAUCGCCAGCGGCGGGGCGCCGCU |
| | | | CAGCAAGGAGGUAGGUGAGGCCGUGGCCAAACGCUUCCACCUACCA |
| | | | GGCAUCCGACAGGGCUACGGCCUGACAGAAACAACCAGCGCCAUUC |
| | | | UGAUCACCCCCGAAGGGGACGACAAGCCUGGCGCAGUAGGCAAGGU |
| | | | GGUGCCCUUCUUCGAGGCUAAGGUGGUGGACUUGGACACCGGUAAG |
| | | | ACACUGGGUGUGAACCAGCGCGGCGAGCUGUGCGUCCGUGGCCCCA |
| | | | UGAUCAUGAGCGGCUACGUUAACAACCCCGAGGCUACAAACGCUCU |
| | | | CAUCGACAAGGACGGCUGGCUGCACAGCGGCGACAUCGCCUACUGG |
| | | | GACGAGGACGAGCACUUCUUCAUCGUGGACCGGCUGAAGUCCCUGA |
| | | | UCAAAUACAAGGGCUACCAGGUAGCCCCAGCCGAACUGGAGAGCAU |
| | | | CCUGCUGCAACACCCCAACAUCUUCGACGCCGGGGUCGCCGGCCUG |
| | | | CCCGACGACGAUGCCGGCGAGCUGCCCGCCGCAGUCGUCGUGCUGG |
| | | | AACACGGUAAAACCAUGACCGAGAAGGAGAUCGUGGACUAUGUGGC |
| | | | CAGCCAGGUUACAACCGCCAAGAAGCUGCGCGGUGGUGUUGUGUUC |
| | | | GUGGACGAGGUGCCUAAAGGACUGACCGGCAAGUUGGACGCCCGCA |
| | | | AGAUCCGCGAGAUUCUCAUUAAGGCCAAGAAGGGCGGCAAGAUCGC |
| | | | CGUGUAAACGCGUGCUAGACCAUGGAUCCUAGACGCUACGCCCCAA |
| | | | UGAUCCGACCAGCAAAACUCGAUGUACUUCCGAGGAACUGAUGUGC |
| | | | AUAAUGCAUCAGGCUGGUACAUUAGAUCCCCGCUUACCGCGGGCAA |
| | | | UAUAGCAACACUAAAAACUCGAUGUACUUCCGAGGAAGCGCAGUGC |
| | | | AUAAUGCUGCGCAGUGUUGCCACAUAACCACUAUAUUAACCAUUUA |
| | | | UCUAGCGGACGCCAAAAACUCAAUGUAUUUCUGAGGAAGCGUGGUG |
| | | | CAUAAUGCCACGCAGCGUCUGCAUAACUUUUAUUAUUUCUUUUAUU |
| | | | AAUCAACAAAAUUUUGUUUUUAACAUUUCAAAAAAAAAAAAAAAAA |
| | | | AAAAAAAAUCUAGAAAAAAAAAAAAAAAAAAAAAAAAAAAAAAAAA |
| | | | AAAAAAAAAAAAAAAAAAAAAAAAAAAAAAAAAAAAAAAAAAAAAA |
| | | | AAAAAAAAAAAAAAAAAAAAA |
| 1782 | mRNA | 1782 | AGGAAACUUAAGUCAACACAACAUAUACAAAACAAACGAAUCUCAA |
| (SEQ | Fluc | | GCAAUCAAGCAUUCUACUUCUAUUGCAGCAAUUUAAAUCAUUUCUU |
| ID | | | UUAAAGCAAAAGCAAUUUUCUGAAAAUUUUCACCAUUUACGAACGA |
| NO: | | | UAGCCAUGGAAGAUGCCAAAAACAUUAAGAAGGGCCCAGCGCCAUU |
| 102) | | | CUACCCACUCGAAGACGGGACCGCCGGCGAGCAGCUGCACAAAGCC |
| | | | AUGAAGCGCUACGCCCUGGUGCCCGGCACCAUCGCCUUUACCGACG |
| | | | CACAUAUCGAGGUGGACAUUACCUACGCCGAGUACUUCGAGAUGAG |
| | | | CGUUCGGCUGGCAGAAGCUAUGAAGCGCUAUGGGCUGAAUACAAAC |
| | | | CAUCGGAUCGUGGUGUGCAGCGAGAAUAGCUUGCAGUUCUUCAUGC |
| | | | CCGUGUUGGGUGCCCUGUUCAUCGGUGUGGCUGUGGCCCCAGCUAA |
| | | | CGACAUCUACAACGAGCGCGAGCUGCUGAACAGCAUGGGCAUCAGC |
| | | | CAGCCCACCGUCGUAUUCGUGAGCAAGAAAGGGCUGCAAAAGAUCC |
| | | | UCAACGUGCAAAAGAAGCUACCGAUCAUACAAAAGAUCAUCAUCAU |
| | | | GGAUAGCAAGACCGACUACCAGGGCUUCCAAAGCAUGUACACCUUC |
| | | | GUGACUUCCCAUUUGCCACCCGGCUUCAACGAGUACGACUUCGUGC |
| | | | CCGAGAGCUUCGACCGGGACAAAACCAUCGCCCUGAUCAUGAACAG |
| | | | UAGUGGCAGUACCGGAUUGCCCAAGGGCGUAGCCCUACCGCACCGC |
| | | | ACCGCUUGUGUCCGAUUCAGUCAUGCCCGCGACCCCAUCUUCGGCA |
| | | | ACCAGAUCAUCCCCGACACCGCUAUCCUCAGCGUGGUGCCAUUUCA |
| | | | CCACGGCUUCGGCAUGUUCACCACGCUGGGCUACUUGAUCUGCGGC |
| | | | UUUCGGGUCGUGCUCAUGUACCGCUUCGAGGAGGAGCUAUUCUUGC |
| | | | GCAGCUUGCAAGACUAUAAGAUUCAAUCUGCCCUGCUGGUGCCCAC |
| | | | ACUAUUUAGCUUCUUCGCUAAGAGCACUCUCAUCGACAAGUACGAC |
| | | | CUAAGCAACUUGCACGAGAUCGCCAGCGGCGGGGCGCCGCUCAGCA |
| | | | AGGAGGUAGGUGAGGCCGUGGCCAAACGCUUCCACCUACCAGGCAU |
| | | | CCGACAGGGCUACGGCCUGACAGAAACAACCAGCGCCAUUCUGAUC |
| | | | ACCCCCGAAGGGGACGACAAGCCUGGCGCAGUAGGCAAGGUGGUGC |
| | | | CCUUCUUCGAGGCUAAGGUGGUGGACUUGGACACCGGUAAGACACU |
| | | | GGGUGUGAACCAGCGCGGCGAGCUGUGCGUCCGUGGCCCCAUGAUC |
| | | | AUGAGCGGCUACGUUAACAACCCCGAGGCUACAAACGCUCUCAUCG |
| | | | ACAAGGACGGCUGGCUGCACAGCGGCGACAUCGCCUACUGGGACGA |
| | | | GGACGAGCACUUCUUCAUCGUGGACCGGCUGAAGUCCCUGAUCAAA |
| | | | UACAAGGGCUACCAGGUAGCCCCAGCCGAACUGGAGAGCAUCCUGC |
| | | | UGCAACACCCCAACAUCUUCGACGCCGGGGUCGCCGGCCUGCCCGA |
| | | | CGACGAUGCCGGCGAGCUGCCCGCCGCAGUCGUCGUGCUGGAACAC |
| | | | GGUAAAACCAUGACCGAGAAGGAGAUCGUGGACUAUGUGGCCAGCC |
| | | | AGGUUACAACCGCCAAGAAGCUGCGCGGUGGUGUUGUGUUCGUGGA |
| | | | CGAGGUGCCUAAAGGACUGACCGGCAAGUUGGACGCCCGCAAGAUC |
| | | | CGCGAGAUUCUCAUUAAGGCCAAGAAGGGCGGCAAGAUCGCCGUGU |
| | | | AACUCGAGCUAGUGACUGACUAGGAUCUGGUUACCACUAAACCAGC |
| | | | CUCAAGAACACCCGAAUGGAGUCUCUAAGCUACAUAAUACCAACUU |
| | | | ACACUUACAAAAUGUUGUCCCCCAAAAUGUAGCCAUUCGUAUCUGC |
| | | | UCCUAAUAAAAAGAAAGUUUCUUCACAUUCUAGAAAAAAAAAAAAA |
| | | | AAAAAAAAAAAAAAAAAAAAAAAAAAAAAAAAAAAAAAAAAAAAAA |
| | | | AAAAAAAAAAAAAAAAAAAAAAAAAAAAAAAAAAAAAAAAAAAAAA |
| | | | AAAAAAAAAAAAAAAA |
| 2847 | STARR™ | 2847 | AUGGGCGGCGCAUGAGAGAAGCCCAGACCAAUUACCUACCCAAAAU |
| (SEQ | KRAS | | GGAGAAAGUUCACGUUGACAUCGAGGAAGACAGCCCAUUCCUCAGA |
| ID | wt | | GCUUUGCAGCGGAGCUUCCCGCAGUUUGAGGUAGAAGCCAAGCAGG |
| NO: | | | UCACUGAUAAUGACCAUGCUAAUGCCAGAGCGUUUUCGCAUCUGGC |
| 103) | | | UUCAAAACUGAUCGAAACGGAGGUGGACCCAUCCGACACGAUCCUU |
| | | | GACAUUGGAAGUGCGCCCGCCCGCAGAAUGUAUUCUAAGCACAAGU |
| | | | AUCAUUGUAUCUGUCCGAUGAGAUGUGCGGAAGAUCCGGACAGAUU |
| | | | GUAUAAGUAUGCAACUAAGCUGAAGAAAAACUGUAAGGAAAUAACU |
| | | | GAUAAGGAAUUGGACAAGAAAAUGAAGGAGCUGGCCGCCGUCAUGA |
| | | | GCGACCCUGACCUGGAAACUGAGACUAUGUGCCUCCACGACGACGA |
| | | | GUCGUGUCGCUACGAAGGGCAAGUCGCUGUUUACCAGGAUGUAUAC |
| | | | GCCGUCGACGGCCCCACCAGCCUGUACCACCAGGCCAACAAGGGCG |
| | | | UGAGGGUGGCCUACUGGAUCGGCUUCGACACCACACCCUUCAUGUU |
| | | | CAAGAACCUGGCCGGCGCCUACCCCAGCUACAGCACCAACUGGGCC |
| | | | GACGAGACCGUGCUGACCGCCAGGAACAUCGGCCUGUGCAGCAGCG |
| | | | ACGUGAUGGAGAGGAGCCGGAGAGGCAUGAGCAUCCUGAGGAAGAA |
| | | | AUACCUGAAGCCCAGCAACAACGUGCUGUUCAGCGUGGGCAGCACC |
| | | | AUCUACCACGAGAAGAGGGACCUGCUCAGGAGCUGGCACCUGCCCA |
| | | | GCGUGUUCCACCUGAGGGGCAAGCAGAACUACACCUGCAGGUGCGA |
| | | | GACCAUCGUGAGCUGCGACGGCUACGUGGUGAAGAGGAUCGCCAUC |
| | | | AGCCCCGGCCUGUACGGCAAGCCCAGCGGCUACGCCGCUACAAUGC |
| | | | ACAGGGAGGGCUUCCUGUGCUGCAAGGUGACCGACACCCUGAACGG |
| | | | CGAGAGGGUGAGCUUCCCCGUGUGCACCUACGUGCCCGCCACCCUG |
| | | | UGCGACCAGAUGACCGGCAUCCUGGCCACCGACGUGAGCGCCGACG |
| | | | ACGCCCAGAAGCUGCUCGUGGGCCUGAACCAGAGGAUCGUGGUCAA |
| | | | CGGCAGGACCCAGAGGAACACCAACACAAUGAAGAACUACCUGCUG |
| | | | CCCGUGGUGGCCCAGGCUUUCGCCAGGUGGGCCAAGGAGUACAAGG |
| | | | AGGACCAGGAAGACGAGAGGCCCCUGGGCCUGAGGGACAGGCAGCU |
| | | | GGUGAUGGGCUGCUGCUGGGCCUUCAGGCGGCACAAGAUCACCAGC |
| | | | AUCUACAAGAGGCCCGACACCCAGACCAUCAUCAAGGUGAACAGCG |
| | | | ACUUCCACAGCUUCGUGCUGCCCAGGAUCGGCAGCAACACCCUGGA |
| | | | GAUCGGCCUGAGGACCCGGAUCAGGAAGAUGCUGGAGGAACACAAG |
| | | | GAGCCCAGCCCACUGAUCACCGCCGAGGACGUGCAGGAGGCCAAGU |
| | | | GCGCUGCCGACGAGGCCAAGGAGGUGAGGGAGGCCGAGGAACUGAG |
| | | | GGCCGCCCUGCCACCCCUGGCUGCCGACGUGGAGGAACCCACCCUG |
| | | | GAAGCCGACGUGGACCUGAUGCUGCAGGAGGCCGGCGCCGGAAGCG |
| | | | UGGAGACACCCAGGGGCCUGAUCAAGGUGACCAGCUACGACGGCGA |
| | | | GGACAAGAUCGGCAGCUACGCCGUGCUGAGCCCACAGGCCGUGCUG |
| | | | AAGUCCGAGAAGCUGAGCUGCAUCCACCCACUGGCCGAGCAGGUGA |
| | | | UCGUGAUCACCCACAGCGGCAGGAAGGGCAGGUACGCCGUGGAGCC |
| | | | CUACCACGGCAAGGUGGUCGUGCCCGAGGGCCACGCCAUCCCCGUG |
| | | | CAGGACUUCCAGGCCCUGAGCGAGAGCGCCACCAUCGUGUACAACG |
| | | | AGAGGGAGUUCGUGAACAGGUACCUGCACCAUAUCGCCACCCACGG |
| | | | CGGAGCCCUGAACACCGACGAGGAAUACUACAAGACCGUGAAGCCC |
| | | | AGCGAGCACGACGGCGAGUACCUGUACGACAUCGACAGGAAGCAGU |
| | | | GCGUGAAGAAAGAGCUGGUGACCGGCCUGGGACUGACCGGCGAGCU |
| | | | GGUGGACCCACCCUUCCACGAGUUCGCCUACGAGAGCCUGAGGACC |
| | | | AGACCCGCCGCUCCCUACCAGGUGCCCACCAUCGGCGUGUACGGCG |
| | | | UGCCCGGCAGCGGAAAGAGCGGCAUCAUCAAGAGCGCCGUGACCAA |
| | | | GAAAGACCUGGUGGUCAGCGCCAAGAAAGAGAACUGCGCCGAGAUC |
| | | | AUCAGGGACGUGAAGAAGAUGAAAGGCCUGGACGUGAACGCGCGCA |
| | | | CCGUGGACAGCGUGCUGCUGAACGGCUGCAAGCACCCCGUGGAGAC |
| | | | CCUGUACAUCGACGAGGCCUUCGCUUGCCACGCCGGCACCCUGAGG |
| | | | GCCCUGAUCGCCAUCAUCAGGCCCAAGAAAGCCGUGCUGUGCGGCG |
| | | | ACCCCAAGCAGUGCGGCUUCUUCAACAUGAUGUGCCUGAAGGUGCA |
| | | | CUUCAACCACGAGAUCUGCACCCAGGUGUUCCACAAGAGCAUCAGC |
| | | | AGGCGGUGCACCAAGAGCGUGACCAGCGUCGUGAGCACCCUGUUCU |
| | | | ACGACAAGAAAAUGAGGACCACCAACCCCAAGGAGACCAAAAUCGU |
| | | | GAUCGACACCACAGGCAGCACCAAGCCCAAGCAGGACGACCUGAUC |
| | | | CUGACCUGCUUCAGGGGCUGGGUGAAGCAGCUGCAGAUCGACUACA |
| | | | AGGGCAACGAGAUCAUGACCGCCGCUGCCAGCCAGGGCCUGACCAG |
| | | | GAAGGGCGUGUACGCCGUGAGGUACAAGGUGAACGAGAACCCACUG |
| | | | UACGCUCCCACCAGCGAGCACGUGAACGUGCUGCUGACCAGGACCG |
| | | | AGGACAGGAUCGUGUGGAAGACCCUGGCCGGCGACCCCUGGAUCAA |
| | | | GACCCUGACCGCCAAGUACCCCGGCAACUUCACCGCCACCAUCGAA |
| | | | GAGUGGCAGGCCGAGCACGACGCCAUCAUGAGGCACAUCCUGGAGA |
| | | | GGCCCGACCCCACCGACGUGUUCCAGAACAAGGCCAACGUGUGCUG |
| | | | GGCCAAGGCCCUGGUGCCCGUGCUGAAGACCGCCGGCAUCGACAUG |
| | | | ACCACAGAGCAGUGGAACACCGUGGACUACUUCGAGACCGACAAGG |
| | | | CCCACAGCGCCGAGAUCGUGCUGAACCAGCUGUGCGUGAGGUUCUU |
| | | | CGGCCUGGACCUGGACAGCGGCCUGUUCAGCGCCCCCACCGUGCCA |
| | | | CUGAGCAUCAGGAACAACCACUGGGACAACAGCCCCAGCCCAAACA |
| | | | UGUACGGCCUGAACAAGGAGGUGGUCAGGCAGCUGAGCAGGCGGUA |
| | | | CCCACAGCUGCCCAGGGCCGUGGCCACCGGCAGGGUGUACGACAUG |
| | | | AACACCGGCACCCUGAGGAACUACGACCCCAGGAUCAACCUGGUGC |
| | | | CCGUGAACAGGCGGCUGCCCCACGCCCUGGUGCUGCACCACAACGA |
| | | | GCACCCACAGAGCGACUUCAGCUCCUUCGUGAGCAAGCUGAAAGGC |
| | | | AGGACCGUGCUGGUCGUGGGCGAGAAGCUGAGCGUGCCCGGCAAGA |
| | | | UGGUGGACUGGCUGAGCGACAGGCCCGAGGCCACCUUCCGGGCCAG |
| | | | GCUGGACCUCGGCAUCCCCGGCGACGUGCCCAAGUACGACAUCAUC |
| | | | UUCGUGAACGUCAGGACCCCAUACAAGUACCACCAUUACCAGCAGU |
| | | | GCGAGGACCACGCCAUCAAGCUGAGCAUGCUGACCAAGAAGGCCUG |
| | | | CCUGCACCUGAACCCCGGAGGCACCUGCGUGAGCAUCGGCUACGGC |
| | | | UACGCCGACAGGGCCAGCGAGAGCAUCAUUGGCGCCAUCGCCAGGC |
| | | | UGUUCAAGUUCAGCAGGGUGUGCAAACCCAAGAGCAGCCUGGAGGA |
| | | | AACCGAGGUGCUGUUCGUGUUCAUCGGCUACGACCGGAAGGCCAGG |
| | | | ACCCACAACCCCUACAAGCUGAGCAGCACCCUGACAAACAUCUACA |
| | | | CCGGCAGCAGGCUGCACGAGGCCGGCUGCGCCCCCAGCUACCACGU |
| | | | GGUCAGGGGCGAUAUCGCCACCGCCACCGAGGGCGUGAUCAUCAAC |
| | | | GCUGCCAACAGCAAGGGCCAGCCCGGAGGCGGAGUGUGCGGCGCCC |
| | | | UGUACAAGAAGUUCCCCGAGAGCUUCGACCUGCAGCCCAUCGAGGU |
| | | | GGGCAAGGCCAGGCUGGUGAAGGGCGCCGCUAAGCACAUCAUCCAC |
| | | | GCCGUGGGCCCCAACUUCAACAAGGUGAGCGAGGUGGAAGGCGACA |
| | | | AGCAGCUGGCCGAAGCCUACGAGAGCAUCGCCAAGAUCGUGAACGA |
| | | | CAAUAACUACAAGAGCGUGGCCAUCCCACUGCUCAGCACCGGCAUC |
| | | | UUCAGCGGCAACAAGGACAGGCUGACCCAGAGCCUGAACCACCUGC |
| | | | UCACCGCCCUGGACACCACCGAUGCCGACGUGGCCAUCUACUGCAG |
| | | | GGACAAGAAGUGGGAGAUGACCCUGAAGGAGGCCGUGGCCAGGCGG |
| | | | GAGGCCGUGGAAGAGAUCUGCAUCAGCGACGACUCCAGCGUGACCG |
| | | | AGCCCGACGCCGAGCUGGUGAGGGUGCACCCCAAGAGCUCCCUGGC |
| | | | CGGCAGGAAGGGCUACAGCACCAGCGACGGCAAGACCUUCAGCUAC |
| | | | CUGGAGGGCACCAAGUUCCACCAGGCCGCUAAGGACAUCGCCGAGA |
| | | | UCAACGCUAUGUGGCCCGUGGCCACCGAGGCCAACGAGCAGGUGUG |
| | | | CAUGUACAUCCUGGGCGAGAGCAUGUCCAGCAUCAGGAGCAAGUGC |
| | | | CCCGUGGAGGAAAGCGAGGCCAGCACACCACCCAGCACCCUGCCCU |
| | | | GCCUGUGCAUCCACGCUAUGACACCCGAGAGGGUGCAGCGGCUGAA |
| | | | GGCCAGCAGGCCCGAGCAGAUCACCGUGUGCAGCUCCUUCCCACUG |
| | | | CCCAAGUACAGGAUCACCGGCGUGCAGAAGAUCCAGUGCAGCCAGC |
| | | | CCAUCCUGUUCAGCCCAAAGGUGCCCGCCUACAUCCACCCCAGGAA |
| | | | GUACCUGGUGGAGACCCCACCCGUGGACGAGACACCCGAGCCAAGC |
| | | | GCCGAGAACCAGAGCACCGAGGGCACACCCGAGCAGCCACCCCUGA |
| | | | UCACCGAGGACGAGACAAGGACCCGGACCCCAGAGCCCAUCAUUAU |
| | | | CGAGGAAGAGGAAGAGGACAGCAUCAGCCUGCUGAGCGACGGCCCC |
| | | | ACCCACCAGGUGCUGCAGGUGGAGGCCGACAUCCACGGCCCACCCA |
| | | | GCGUGUCCAGCUCCAGCUGGAGCAUCCCACACGCCAGCGACUUCGA |
| | | | CGUGGACAGCCUGAGCAUCCUGGACACCCUGGAGGGCGCCAGCGUG |
| | | | ACCUCCGGCGCCACCAGCGCCGAGACCAACAGCUACUUCGCCAAGA |
| | | | GCAUGGAGUUCCUGGCCAGGCCCGUGCCAGCUCCCAGGACCGUGUU |
| | | | CAGGAACCCACCCCACCCAGCUCCCAGGACCAGGACCCCAAGCCUG |
| | | | GCUCCCAGCAGGGCCUGCAGCAGGACCAGCCUGGUGAGCACCCCAC |
| | | | CCGGCGUGAACAGGGUGAUCACCAGGGAGGAACUGGAGGCCCUGAC |
| | | | ACCCAGCAGGACCCCCAGCAGGUCCGUGAGCAGGACUAGUCUGGUG |
| | | | UCCAACCCACCCGGCGUGAACAGGGUGAUCACCAGGGAGGAAUUCG |
| | | | AGGCCUUCGUGGCCCAGCAACAGAGACGGUUCGACGCCGGCGCCUA |
| | | | CAUCUUCAGCAGCGACACCGGCCAGGGACACCUGCAGCAAAAGAGC |
| | | | GUGAGGCAGACCGUGCUGAGCGAGGUGGUGCUGGAGAGGACCGAGC |
| | | | UGGAAAUCAGCUACGCCCCCAGGCUGGACCAGGAGAAGGAGGAACU |
| | | | GCUCAGGAAGAAACUGCAGCUGAACCCCACCCCAGCCAACAGGAGC |
| | | | AGGUACCAGAGCAGGAAGGUGGAGAACAUGAAGGCCAUCACCGCCA |
| | | | GGCGGAUCCUGCAGGGCCUGGGACACUACCUGAAGGCCGAGGGCAA |
| | | | GGUGGAGUGCUACAGGACCCUGCACCCCGUGCCACUGUACAGCUCC |
| | | | AGCGUGAACAGGGCCUUCUCCAGCCCCAAGGUGGCCGUGGAGGCCU |
| | | | GCAACGCUAUGCUGAAGGAGAACUUCCCCACCGUGGCCAGCUACUG |
| | | | CAUCAUCCCCGAGUACGACGCCUACCUGGACAUGGUGGACGGCGCC |
| | | | AGCUGCUGCCUGGACACCGCCAGCUUCUGCCCCGCCAAGCUGAGGA |
| | | | GCUUCCCCAAGAAACACAGCUACCUGGAGCCCACCAUCAGGAGCGC |
| | | | CGUGCCCAGCGCCAUCCAGAACACCCUGCAGAACGUGCUGGCCGCU |
| | | | GCCACCAAGAGGAACUGCAACGUGACCCAGAUGAGGGAGCUGCCCG |
| | | | UGCUGGACAGCGCUGCCUUCAACGUGGAGUGCUUCAAGAAAUACGC |
| | | | CUGCAACAACGAGUACUGGGAGACCUUCAAGGAGAACCCCAUCAGG |
| | | | CUGACCGAAGAGAACGUGGUGAACUACAUCACCAAGCUGAAGGGCC |
| | | | CCAAGGCCGCUGCCCUGUUCGCUAAGACCCACAACCUGAACAUGCU |
| | | | GCAGGACAUCCCAAUGGACAGGUUCGUGAUGGACCUGAAGAGGGAC |
| | | | GUGAAGGUGACACCCGGCACCAAGCACACCGAGGAGAGGCCCAAGG |
| | | | UGCAGGUGAUCCAGGCCGCUGACCCACUGGCCACCGCCUACCUGUG |
| | | | CGGCAUCCACAGGGAGCUGGUGAGGCGGCUGAACGCCGUGCUGCUG |
| | | | CCCAACAUCCACACCCUGUUCGACAUGAGCGCCGAGGACUUCGACG |
| | | | CCAUCAUCGCCGAGCACUUCCAGCCCGGCGACUGCGUGCUGGAGAC |
| | | | CGACAUCGCCAGCUUCGACAAGAGCGAGGAUGACGCUAUGGCCCUG |
| | | | ACCGCUCUGAUGAUCCUGGAGGACCUGGGCGUGGACGCCGAGCUGC |
| | | | UCACCCUGAUCGAGGCUGCCUUCGGCGAGAUCAGCUCCAUCCACCU |
| | | | GCCCACCAAGACCAAGUUCAAGUUCGGCGCUAUGAUGAAAAGCGGA |
| | | | AUGUUCCUGACCCUGUUCGUGAACACCGUGAUCAACAUUGUGAUCG |
| | | | CCAGCAGGGUGCUGCGGGAGAGGCUGACCGGCAGCCCCUGCGCUGC |
| | | | CUUCAUCGGCGACGACAACAUCGUGAAGGGCGUGAAAAGCGACAAG |
| | | | CUGAUGGCCGACAGGUGCGCCACCUGGCUGAACAUGGAGGUGAAGA |
| | | | UCAUCGACGCCGUGGUGGGCGAGAAGGCCCCCUACUUCUGCGGCGG |
| | | | AUUCAUCCUGUGCGACAGCGUGACCGGCACCGCCUGCAGGGUGGCC |
| | | | GACCCCCUGAAGAGGCUGUUCAAGCUGGGCAAGCCACUGGCCGCUG |
| | | | ACGAUGAGCACGACGAUGACAGGCGGAGGGCCCUGCACGAGGAAAG |
| | | | CACCAGGUGGAACAGGGUGGGCAUCCUGAGCGAGCUGUGCAAGGCC |
| | | | GUGGAGAGCAGGUACGAGACCGUGGGCACCAGCAUCAUCGUGAUGG |
| | | | CUAUGACCACACUGGCCAGCUCCGUCAAGAGCUUCUCCUACCUGAG |
| | | | GGGGGCCCCUAUAACUCUCUACGGCUAACCUGAAUGGACUACGACA |
| | | | UAGUCUAGUCCGCCAAGGCCGCCACCCAUGAAGUUGGUGGUUGUGG |
| | | | GGGCCGGGGGUGUUGGCAAAAGCGCCCUUACAAUUUGACUCGAGUA |
| | | | UGUUACGUGCAAAGGUGAUUGUCACCCCCCGAAAGACCAUAUUGUG |
| | | | ACACACCCUCAGUAUCACGCCCAAACAUUUACAGCCGCGGUGUCAA |
| | | | AAACCGCGUGGACGUGGUUAACAUCCCUGCUGGGAGGAUCAGCCGU |
| | | | AAUUAUUAUAAUUGGCUUGGUGCUGGCUACUAUUGUGGCCAUGUAC |
| | | | GUGCUGACCAACCAGAAACAUAAUUGAAUACAGCAGCAAUUGGCAA |
| | | | GCUGCUUACAUAGAACUCGCGGCGAUUGGCAUGCCGCCUUAAAAUU |
| | | | UUUAUUUUAUUUUUUCUUUUCUUUUCCGAAUCGGAUUUUGUUUUUA |
| | | | AUAUUUCAAAAAAAAAAAAAAAAAAAAAAAAAUCUAGAAAAAAAAA |
| | | | AAAAAAAAAAAAAAAAAAAAAAAAAAAAAAAAAAAAAAAAAAAAAA |
| | | | AAAAAAAAAAAAAAAAAAAAAAAAAAAAAAAAAAAAAAAAAAAAA |
| 2862 | SINV | 2862 | AUUGACGGCGUAGUACACACUAUUGAAUCAAACAGCCGACCAAUUG |
| (SEQ | empty | | CACUACCAUCACAAUGGAGAAGCCAGUAGUAAACGUAGACGUAGAC |
| ID | | | CCCCAGAGUCCGUUUGUCGUGCAACUGCAAAAAAGCUUCCCGCAAU |
| NO: | | | UUGAGGUAGUAGCACAGCAGGUCACUCCAAAUGACCAUGCUAAUGC |
| 104) | | | CAGAGCAUUUUCGCAUCUGGCCAGUAAACUAAUCGAGCUGGAGGUU |
| | | | CCUACCACAGCGACGAUCUUGGACAUAGGCAGCGCACCGGCUCGUA |
| | | | GAAUGUUUUCCGAGCACCAGUAUCAUUGUGUCUGCCCCAUGCGUAG |
| | | | UCCAGAAGACCCGGACCGCAUGAUGAAAUAUGCCAGUAAACUGGCG |
| | | | GAAAAAGCGUGCAAGAUUACAAACAAGAACUUGCAUGAGAAGAUUA |
| | | | AGGAUCUCCGGACCGUACUUGAUACGCCGGAUGCUGAAACACCAUC |
| | | | GCUCUGCUUUCACAACGAUGUUACCUGCAACAUGCGUGCCGAAUAU |
| | | | UCCGUCAUGCAGGACGUGUAUAUCAACGCUCCCGGAACUAUCUAUC |
| | | | AUCAGGCUAUGAAAGGCGUGCGGACCCUGUACUGGAUUGGCUUCGA |
| | | | CACCACCCAGUUCAUGUUCUCGGCUAUGGCAGGUUCGUACCCUGCG |
| | | | UACAACACCAACUGGGCCGACGAGAAAGUCCUUGAAGCGCGUAACA |
| | | | UCGGACUUUGCAGCACAAAGCUGAGUGAAGGUAGGACAGGAAAAUU |
| | | | GUCGAUAAUGAGGAAGAAGGAGUUGAAGCCCGGGUCGCGGGUUUAU |
| | | | UUCUCCGUAGGAUCGACACUUUAUCCAGAACACAGAGCCAGCUUGC |
| | | | AGAGCUGGCAUCUUCCAUCGGUGUUCCACUUGAAUGGAAAGCAGUC |
| | | | GUACACUUGCCGCUGUGAUACAGUGGUGAGUUGCGAAGGCUACGUA |
| | | | GUGAAGAAAAUCACCAUCAGUCCCGGGAUCACGGGAGAAACCGUGG |
| | | | GAUACGCGGUUACACACAAUAGCGAGGGCUUCUUGCUAUGCAAAGU |
| | | | UACUGACACAGUAAAAGGAGAACGGGUAUCGUUCCCUGUGUGCACG |
| | | | UACAUCCCGGCCACCAUAUGCGAUCAGAUGACUGGUAUAAUGGCCA |
| | | | CGGAUAUAUCACCUGACGAUGCACAAAAACUUCUGGUUGGGCUCAA |
| | | | CCAGCGAAUUGUCAUUAACGGUAGGACUAACAGGAACACCAACACC |
| | | | AUGCAAAAUUACCUUCUGCCGAUCAUAGCACAAGGGUUCAGCAAAU |
| | | | GGGCUAAGGAGCGCAAGGAUGAUCUUGAUAACGAGAAAAUGCUGGG |
| | | | UACUAGAGAACGCAAGCUUACGUAUGGCUGCUUGUGGGCGUUUCGC |
| | | | ACUAAGAAAGUACAUUCGUUUUAUCGCCCACCUGGAACGCAGACCU |
| | | | GCGUAAAAGUCCCAGCCUCUUUUAGCGCUUUUCCCAUGUCGUCCGU |
| | | | AUGGACGACCUCUUUGCCCAUGUCGCUGAGGCAGAAAUUGAAACUG |
| | | | GCAUUGCAACCAAAGAAGGAGGAAAAACUGCUGCAGGUCUCGGAGG |
| | | | AAUUAGUCAUGGAGGCCAAGGCUGCUUUUGAGGAUGCUCAGGAGGA |
| | | | AGCCAGAGCGGAGAAGCUCCGAGAAGCACUUCCACCAUUAGUGGCA |
| | | | GACAAAGGCAUCGAGGCAGCCGCAGAAGUUGUCUGCGAAGUGGAGG |
| | | | GGCUCCAGGCGGACAUCGGAGCAGCAUUAGUUGAAACCCCGCGCGG |
| | | | UCACGUAAGGAUAAUACCUCAAGCAAAUGACCGUAUGAUCGGACAG |
| | | | UAUAUCGUUGUCUCGCCAAACUCUGUGCUGAAGAAUGCCAAACUCG |
| | | | CACCAGCGCACCCGCUAGCAGAUCAGGUUAAGAUCAUAACACACUC |
| | | | CGGAAGAUCAGGAAGGUACGCGGUCGAACCAUACGACGCUAAAGUA |
| | | | CUGAUGCCAGCAGGAGGUGCCGUACCAUGGCCAGAAUUCCUAGCAC |
| | | | UGAGUGAGAGCGCCACGUUAGUGUACAACGAAAGAGAGUUUGUGAA |
| | | | CCGCAAACUAUACCACAUUGCCAUGCAUGGCCCCGCCAAGAAUACA |
| | | | GAAGAGGAGCAGUACAAGGUUACAAAGGCAGAGCUUGCAGAAACAG |
| | | | AGUACGUGUUUGACGUGGACAAGAAGCGUUGCGUUAAGAAGGAAGA |
| | | | AGCCUCAGGUCUGGUCCUCUCGGGAGAACUGACCAACCCUCCCUAU |
| | | | CAUGAGCUAGCUCUGGAGGGACUGAAGACCCGACCUGCGGUCCCGU |
| | | | ACAAGGUCGAAACAAUAGGAGUGAUAGGCACACCGGGGUCGGGCAA |
| | | | GUCAGCUAUUAUCAAGUCAACUGUCACGGCACGAGAUCUUGUUACC |
| | | | AGCGGAAAGAAAGAAAAUUGUCGCGAAAUUGAGGCCGACGUGCUAA |
| | | | GACUGAGGGGUAUGCAGAUUACGUCGAAGACAGUAGAUUCGGUUAU |
| | | | GCUCAACGGAUGCCACAAAGCCGUAGAAGUGCUGUACGUUGACGAA |
| | | | GCGUUCGCGUGCCACGCAGGAGCACUACUUGCCUUGAUUGCUAUCG |
| | | | UCAGGCCCCGCAAGAAGGUAGUACUAUGCGGAGACCCCAUGCAAUG |
| | | | CGGAUUCUUCAACAUGAUGCAACUAAAGGUACAUUUCAAUCACCCU |
| | | | GAAAAAGACAUAUGCACCAAGACAUUCUACAAGUAUAUCUCCCGGC |
| | | | GUUGCACACAGCCAGUUACAGCUAUUGUAUCGACACUGCAUUACGA |
| | | | UGGAAAGAUGAAAACCACGAACCCGUGCAAGAAGAACAUUGAAAUC |
| | | | GAUAUUACAGGGGCCACAAAGCCGAAGCCAGGGGAUAUCAUCCUGA |
| | | | CAUGUUUCCGCGGGUGGGUUAAGCAAUUGCAAAUCGACUAUCCCGG |
| | | | ACAUGAAGUAAUGACAGCCGCGGCCUCACAAGGGCUAACCAGAAAA |
| | | | GGAGUGUAUGCCGUCCGGCAAAAAGUCAAUGAAAACCCACUGUACG |
| | | | CGAUCACAUCAGAGCAUGUGAACGUGUUGCUCACCCGCACUGAGGA |
| | | | CAGGCUAGUGUGGAAAACCUUGCAGGGCGACCCAUGGAUUAAGCAG |
| | | | CUCACUAACAUACCUAAAGGAAACUUUCAGGCUACUAUAGAGGACU |
| | | | GGGAAGCUGAACACAAGGGAAUAAUUGCUGCAAUAAACAGCCCCAC |
| | | | UCCCCGUGCCAAUCCGUUCAGCUGCAAGACCAACGUUUGCUGGGCG |
| | | | AAAGCAUUGGAACCGAUACUAGCCACGGCCGGUAUCGUACUUACCG |
| | | | GUUGCCAGUGGAGCGAACUGUUCCCACAGUUUGCGGAUGACAAACC |
| | | | ACAUUCGGCCAUUUACGCCUUAGACGUAAUUUGCAUUAAGUUUUUC |
| | | | GGCAUGGACUUGACAAGCGGACUGUUUUCUAAACAGAGCAUCCCAC |
| | | | UAACGUACCAUCCCGCCGAUUCAGCGAGGCCGGUAGCUCAUUGGGA |
| | | | CAACAGCCCAGGAACCCGCAAGUAUGGGUACGAUCACGCCAUUGCC |
| | | | GCCGAACUCUCCCGUAGAUUUCCGGUGUUCCAGCUAGCUGGGAAGG |
| | | | GCACACAACUUGAUUUGCAGACGGGGAGAACCAGAGUUAUCUCUGC |
| | | | ACAGCAUAACCUGGUCCCGGUGAACCGCAAUCUUCCUCACGCCUUA |
| | | | GUCCCCGAGUACAAGGAGAAGCAACCCGGCCCGGUCGAAAAAUUCU |
| | | | UGAACCAGUUCAAACACCACUCAGUACUUGUGGUAUCAGAGGAAAA |
| | | | AAUUGAAGCUCCCCGUAAGAGAAUCGAAUGGAUCGCCCCGAUUGGC |
| | | | AUAGCCGGUGCAGAUAAGAACUACAACCUGGCUUUCGGGUUUCCGC |
| | | | CGCAGGCACGGUACGACCUGGUGUUCAUCAACAUUGGAACUAAAUA |
| | | | CAGAAACCACCACUUUCAGCAGUGCGAAGACCAUGCGGCGACCUUA |
| | | | AAAACCCUUUCGCGUUCGGCCCUGAAUUGCCUUAACCCAGGAGGCA |
| | | | CCCUCGUGGUGAAGUCCUAUGGCUACGCCGACCGCAACAGUGAGGA |
| | | | CGUAGUCACCGCUCUUGCCAGAAAGUUUGUCAGGGUGUCUGCAGCG |
| | | | AGACCAGAUUGUGUCUCAAGCAAUACAGAAAUGUACCUGAUUUUCC |
| | | | GACAACUAGACAACAGCCGUACACGGCAAUUCACCCCGCACCAUCU |
| | | | GAAUUGCGUGAUUUCGUCCGUGUAUGAGGGUACAAGAGAUGGAGUU |
| | | | GGAGCCGCGCCGUCAUACCGCACCAAAAGGGAGAAUAUUGCUGACU |
| | | | GUCAAGAGGAAGCAGUUGUCAACGCAGCCAAUCCGCUGGGUAGACC |
| | | | AGGCGAAGGAGUCUGCCGUGCCAUCUAUAAACGUUGGCCGACCAGU |
| | | | UUUACCGAUUCAGCCACGGAGACAGGCACCGCAAGAAUGACUGUGU |
| | | | GCCUAGGAAAGAAAGUGAUCCACGCGGUCGGCCCUGAUUUCCGGAA |
| | | | GCACCCAGAAGCAGAAGCCUUGAAAUUGCUACAAAACGCCUACCAU |
| | | | GCAGUGGCAGACUUAGUAAAUGAACAUAACAUCAAGUCUGUCGCCA |
| | | | UUCCACUGCUAUCUACAGGCAUUUACGCAGCCGGAAAAGACCGCCU |
| | | | UGAAGUAUCACUUAACUGCUUGACAACCGCGCUAGACAGAACUGAC |
| | | | GCGGACGUAACCAUCUAUUGCCUGGAUAAGAAGUGGAAGGAAAGAA |
| | | | UCGACGCGGCACUCCAACUUAAGGAGUCUGUAACAGAGCUGAAGGA |
| | | | UGAAGAUAUGGAGAUCGACGAUGAGUUAGUAUGGAUCCAUCCAGAC |
| | | | AGUUGCUUGAAGGGAAGAAAGGGAUUCAGUACUACAAAAGGAAAAU |
| | | | UGUAUUCGUACUUCGAAGGCACCAAAUUCCAUCAAGCAGCAAAAGA |
| | | | CAUGGCGGAGAUAAAGGUCCUGUUCCCUAAUGACCAGGAAAGUAAU |
| | | | GAACAACUGUGUGCCUACAUAUUGGGUGAGACCAUGGAAGCAAUCC |
| | | | GCGAAAAGUGCCCGGUCGACCAUAACCCGUCGUCUAGCCCGCCCAA |
| | | | AACGUUGCCGUGCCUUUGCAUGUAUGCCAUGACGCCAGAAAGGGUC |
| | | | CACAGACUUAGAAGCAAUAACGUCAAAGAAGUUACAGUAUGCUCCU |
| | | | CCACCCCCCUUCCUAAGCACAAAAUUAAGAAUGUUCAGAAGGUUCA |
| | | | GUGCACGAAAGUAGUCCUGUUUAAUCCGCACACUCCCGCAUUCGUU |
| | | | CCCGCCCGUAAGUACAUAGAAGUGCCAGAACAGCCUACCGCUCCUC |
| | | | CUGCACAGGCCGAGGAGGCCCCCGAAGUUGUAGCGACACCGUCACC |
| | | | AUCUACAGCUGAUAACACCUCGCUUGAUGUCACAGACAUCUCACUG |
| | | | GAUAUGGAUGACAGUAGCGAAGGCUCACUUUUUUCGAGCUUUAGCG |
| | | | GAUCGGACAACUCUAUUACUAGUAUGGACAGUUGGUCGUCAGGACC |
| | | | UAGUUCACUAGAGAUAGUAGACCGAAGGCAGGUGGUGGUGGCUGAC |
| | | | GUUCAUGCCGUCCAAGAGCCUGCCCCUAUUCCACCGCCAAGGCUAA |
| | | | AGAAGAUGGCCCGCCUGGCAGCGGCAAGAAAAGAGCCCACUCCACC |
| | | | GGCAAGCAAUAGCUCUGAGUCCCUCCACCUCUCUUUUGGUGGGGUA |
| | | | UCCAUGUCCCUCGGAUCAAUUUUCGACGGAGAGACGGCCCGCCAGG |
| | | | CAGCGGUACAACCCCUGGCAACAGGCCCCACGGAUGUGCCUAUGUC |
| | | | UUUCGGAUCGUUUUCCGACGGAGAGAUUGAUGAGCUGAGCCGCAGA |
| | | | GUAACUGAGUCCGAACCCGUCCUGUUUGGAUCAUUUGAACCGGGCG |
| | | | AAGUGAACUCAAUUAUAUCGUCCCGAUCAGCCGUAUCUUUUCCUCU |
| | | | ACGCAAGCAGAGACGUAGACGCAGGAGCAGGAGGACUGAAUACUGA |
| | | | CUAACCGGGGUAGGUGGGUACAUAUUUUCGACGGACACAGGCCCUG |
| | | | GGCACUUGCAAAAGAAGUCCGUUCUGCAGAACCAGCUUACAGAACC |
| | | | GACCUUGGAGCGCAAUGUCCUGGAAAGAAUUCAUGCCCCGGUGCUC |
| | | | GACACGUCGAAAGAGGAACAACUCAAACUCAGGUACCAGAUGAUGC |
| | | | CCACCGAAGCCAACAAAAGUAGGUACCAGUCUCGUAAAGUAGAAAA |
| | | | UCAGAAAGCCAUAACCACUGAGCGACUACUGUCAGGACUACGACUG |
| | | | UAUAACUCUGCCACAGAUCAGCCAGAAUGCUAUAAGAUCACCUAUC |
| | | | CGAAACCAUUGUACUCCAGUAGCGUACCGGCGAACUACUCCGAUCC |
| | | | ACAGUUCGCUGUAGCUGUCUGUAACAACUAUCUGCAUGAGAACUAU |
| | | | CCGACAGUAGCAUCUUAUCAGAUUACUGACGAGUACGAUGCUUACU |
| | | | UGGAUAUGGUAGACGGGACAGUCGCCUGCCUGGACACUGCAACCUU |
| | | | CUGCCCCGCUAAGCUUAGAAGUUACCCGAAAAAACAUGAGUAUAGA |
| | | | GCCCCGAAUAUCCGCAGUGCGGUUCCAUCAGCGAUGCAGAACACGC |
| | | | UACAAAAUGUGCUCAUUGCCGCAACUAAAAGAAAUUGCAACGUCAC |
| | | | GCAGAUGCGUGAACUGCCAACACUGGACUCAGCGACAUUCAAUGUC |
| | | | GAAUGCUUUCGAAAAUAUGCAUGUAAUGACGAGUAUUGGGAGGAGU |
| | | | UCGCUCGGAAGCCAAUUAGGAUUACCACUGAGUUUGUCACCGCAUA |
| | | | UGUAGCUAGACUGAAAGGCCCUAAGGCCGCCGCACUAUUUGCAAAG |
| | | | ACGUAUAAUUUGGUCCCAUUGCAAGAAGUGCCUAUGGAUAGAUUCG |
| | | | UCAUGGACAUGAAAAGAGACGUGAAAGUUACACCAGGCACGAAACA |
| | | | CACAGAAGAAAGACCGAAAGUACAAGUGAUACAAGCCGCAGAACCC |
| | | | CUGGCGACUGCUUACUUAUGCGGGAUUCACCGGGAAUUAGUGCGUA |
| | | | GGCUUACGGCCGUCUUGCUUCCAAACAUUCACACGCUUUUUGACAU |
| | | | GUCGGCGGAGGAUUUUGAUGCAAUCAUAGCAGAACACUUCAAGCAA |
| | | | GGCGACCCGGUACUGGAGACGGAUAUCGCAUCAUUCGACAAAAGCC |
| | | | AAGACGACGCUAUGGCGUUAACCGGUCUGAUGAUCUUGGAGGACCU |
| | | | GGGUGUGGAUCAACCACUACUCGACUUGAUCGAGUGCGCCUUUGGA |
| | | | GAAAUAUCAUCCACCCAUCUACCUACGGGUACUCGUUUUAAAUUCG |
| | | | GGGCGAUGAUGAAAUCCGGAAUGUUCCUCACACUUUUUGUCAACAC |
| | | | AGUUUUGAAUGUCGUUAUCGCCAGCAGAGUACUAGAGGAGCGGCUU |
| | | | AAAACGUCCAGAUGUGCAGCGUUCAUUGGCGACGACAACAUCAUAC |
| | | | AUGGAGUAGUAUCUGACAAAGAAAUGGCUGAGAGGUGCGCCACCUG |
| | | | GCUCAACAUGGAGGUUAAGAUCAUCGACGCAGUCAUCGGUGAGAGA |
| | | | CCACCUUACUUCUGCGGCGGAUUUAUCUUGCAAGAUUCGGUUACUU |
| | | | CCACAGCGUGCCGCGUGGCGGAUCCCCUGAAAAGGCUGUUUAAGUU |
| | | | GGGUAAACCGCUCCCAGCCGACGACGAGCAAGACGAAGACAGAAGA |
| | | | CGCGCUCUGCUAGAUGAAACAAAGGCGUGGUUUAGAGUAGGUAUAA |
| | | | CAGGCACUUUAGCAGUGGCCGUGACGACCCGGUAUGAGGUAGACAA |
| | | | UAUUACACCUGUCCUACUGGCAUUGAGAACUUUUGCCCAGAGCAAA |
| | | | AGAGCAUUCCAAGCCAUCAGAGGGGAAAUAAAGCAUCUCUACGGUG |
| | | | GUCCUAAAUAGUCAGCAUAGUACAUUUCAUCUGACUAAUACUACAA |
| | | | CACCACCACCACGCGUGCUAGACCAUGGAUCCUAGACGCUACGCCC |
| | | | CAAUGAUCCGACCAGCAAAACUCGAUGUACUUCCGAGGAACUGAUG |
| | | | UGCAUAAUGCAUCAGGCUGGUACAUUAGAUCCCCGCUUACCGCGGG |
| | | | CAAUAUAGCAACACUAAAAACUCGAUGUACUUCCGAGGAAGCGCAG |
| | | | UGCAUAAUGCUGCGCAGUGUUGCCACAUAACCACUAUAUUAACCAU |
| | | | UUAUCUAGCGGACGCCAAAAACUCAAUGUAUUUCUGAGGAAGCGUG |
| | | | GUGCAUAAUGCCACGCAGCGUCUGCAUAACUUUUAUUAUUUCUUUU |
| | | | AUUAAUCAACAAAAUUUUGUUUUUAACAUUUCAAAAAAAAAAAAAA |
| | | | AAAAAAAAAAAUCUAGAAAAAAAAAAAAAAAAAAAAAAAAAAAAAA |
| | | | AAAAAAAAAAAAAAAAAAAAAAAAAAAAAAAAAAAAAAAAAAAAAA |
| | | | AAAAAAAAAAAAAAAAAAAAAAAA |
| 3060 | STARR™ | 3060 | AUGGGCGGCGCAUGAGAGAAGCCCAGACCAAUUACCUACCCAAAAU |
| (SEQ | gp70 | | GGAGAAAGUUCACGUUGACAUCGAGGAAGACAGCCCAUUCCUCAGA |
| ID | | | GCUUUGCAGCGGAGCUUCCCGCAGUUUGAGGUAGAAGCCAAGCAGG |
| NO: | | | UCACUGAUAAUGACCAUGCUAAUGCCAGAGCGUUUUCGCAUCUGGC |
| 105) | | | UUCAAAACUGAUCGAAACGGAGGUGGACCCAUCCGACACGAUCCUU |
| | | | GACAUUGGAAGUGCGCCCGCCCGCAGAAUGUAUUCUAAGCACAAGU |
| | | | AUCAUUGUAUCUGUCCGAUGAGAUGUGCGGAAGAUCCGGACAGAUU |
| | | | GUAUAAGUAUGCAACUAAGCUGAAGAAAAACUGUAAGGAAAUAACU |
| | | | GAUAAGGAAUUGGACAAGAAAAUGAAGGAGCUGGCCGCCGUCAUGA |
| | | | GCGACCCUGACCUGGAAACUGAGACUAUGUGCCUCCACGACGACGA |
| | | | GUCGUGUCGCUACGAAGGGCAAGUCGCUGUUUACCAGGAUGUAUAC |
| | | | GCCGUCGACGGCCCCACCAGCCUGUACCACCAGGCCAACAAGGGCG |
| | | | UGAGGGUGGCCUACUGGAUCGGCUUCGACACCACACCCUUCAUGUU |
| | | | CAAGAACCUGGCCGGCGCCUACCCCAGCUACAGCACCAACUGGGCC |
| | | | GACGAGACCGUGCUGACCGCCAGGAACAUCGGCCUGUGCAGCAGCG |
| | | | ACGUGAUGGAGAGGAGCCGGAGAGGCAUGAGCAUCCUGAGGAAGAA |
| | | | AUACCUGAAGCCCAGCAACAACGUGCUGUUCAGCGUGGGCAGCACC |
| | | | AUCUACCACGAGAAGAGGGACCUGCUCAGGAGCUGGCACCUGCCCA |
| | | | GCGUGUUCCACCUGAGGGGCAAGCAGAACUACACCUGCAGGUGCGA |
| | | | GACCAUCGUGAGCUGCGACGGCUACGUGGUGAAGAGGAUCGCCAUC |
| | | | AGCCCCGGCCUGUACGGCAAGCCCAGCGGCUACGCCGCUACAAUGC |
| | | | ACAGGGAGGGCUUCCUGUGCUGCAAGGUGACCGACACCCUGAACGG |
| | | | CGAGAGGGUGAGCUUCCCCGUGUGCACCUACGUGCCCGCCACCCUG |
| | | | UGCGACCAGAUGACCGGCAUCCUGGCCACCGACGUGAGCGCCGACG |
| | | | ACGCCCAGAAGCUGCUCGUGGGCCUGAACCAGAGGAUCGUGGUCAA |
| | | | CGGCAGGACCCAGAGGAACACCAACACAAUGAAGAACUACCUGCUG |
| | | | CCCGUGGUGGCCCAGGCUUUCGCCAGGUGGGCCAAGGAGUACAAGG |
| | | | AGGACCAGGAAGACGAGAGGCCCCUGGGCCUGAGGGACAGGCAGCU |
| | | | GGUGAUGGGCUGCUGCUGGGCCUUCAGGCGGCACAAGAUCACCAGC |
| | | | AUCUACAAGAGGCCCGACACCCAGACCAUCAUCAAGGUGAACAGCG |
| | | | ACUUCCACAGCUUCGUGCUGCCCAGGAUCGGCAGCAACACCCUGGA |
| | | | GAUCGGCCUGAGGACCCGGAUCAGGAAGAUGCUGGAGGAACACAAG |
| | | | GAGCCCAGCCCACUGAUCACCGCCGAGGACGUGCAGGAGGCCAAGU |
| | | | GCGCUGCCGACGAGGCCAAGGAGGUGAGGGAGGCCGAGGAACUGAG |
| | | | GGCCGCCCUGCCACCCCUGGCUGCCGACGUGGAGGAACCCACCCUG |
| | | | GAAGCCGACGUGGACCUGAUGCUGCAGGAGGCCGGCGCCGGAAGCG |
| | | | UGGAGACACCCAGGGGCCUGAUCAAGGUGACCAGCUACGACGGCGA |
| | | | GGACAAGAUCGGCAGCUACGCCGUGCUGAGCCCACAGGCCGUGCUG |
| | | | AAGUCCGAGAAGCUGAGCUGCAUCCACCCACUGGCCGAGCAGGUGA |
| | | | UCGUGAUCACCCACAGCGGCAGGAAGGGCAGGUACGCCGUGGAGCC |
| | | | CUACCACGGCAAGGUGGUCGUGCCCGAGGGCCACGCCAUCCCCGUG |
| | | | CAGGACUUCCAGGCCCUGAGCGAGAGCGCCACCAUCGUGUACAACG |
| | | | AGAGGGAGUUCGUGAACAGGUACCUGCACCAUAUCGCCACCCACGG |
| | | | CGGAGCCCUGAACACCGACGAGGAAUACUACAAGACCGUGAAGCCC |
| | | | AGCGAGCACGACGGCGAGUACCUGUACGACAUCGACAGGAAGCAGU |
| | | | GCGUGAAGAAAGAGCUGGUGACCGGCCUGGGACUGACCGGCGAGCU |
| | | | GGUGGACCCACCCUUCCACGAGUUCGCCUACGAGAGCCUGAGGACC |
| | | | AGACCCGCCGCUCCCUACCAGGUGCCCACCAUCGGCGUGUACGGCG |
| | | | UGCCCGGCAGCGGAAAGAGCGGCAUCAUCAAGAGCGCCGUGACCAA |
| | | | GAAAGACCUGGUGGUCAGCGCCAAGAAAGAGAACUGCGCCGAGAUC |
| | | | AUCAGGGACGUGAAGAAGAUGAAAGGCCUGGACGUGAACGCGCGCA |
| | | | CCGUGGACAGCGUGCUGCUGAACGGCUGCAAGCACCCCGUGGAGAC |
| | | | CCUGUACAUCGACGAGGCCUUCGCUUGCCACGCCGGCACCCUGAGG |
| | | | GCCCUGAUCGCCAUCAUCAGGCCCAAGAAAGCCGUGCUGUGCGGCG |
| | | | ACCCCAAGCAGUGCGGCUUCUUCAACAUGAUGUGCCUGAAGGUGCA |
| | | | CUUCAACCACGAGAUCUGCACCCAGGUGUUCCACAAGAGCAUCAGC |
| | | | AGGCGGUGCACCAAGAGCGUGACCAGCGUCGUGAGCACCCUGUUCU |
| | | | ACGACAAGAAAAUGAGGACCACCAACCCCAAGGAGACCAAAAUCGU |
| | | | GAUCGACACCACAGGCAGCACCAAGCCCAAGCAGGACGACCUGAUC |
| | | | CUGACCUGCUUCAGGGGCUGGGUGAAGCAGCUGCAGAUCGACUACA |
| | | | AGGGCAACGAGAUCAUGACCGCCGCUGCCAGCCAGGGCCUGACCAG |
| | | | GAAGGGCGUGUACGCCGUGAGGUACAAGGUGAACGAGAACCCACUG |
| | | | UACGCUCCCACCAGCGAGCACGUGAACGUGCUGCUGACCAGGACCG |
| | | | AGGACAGGAUCGUGUGGAAGACCCUGGCCGGCGACCCCUGGAUCAA |
| | | | GACCCUGACCGCCAAGUACCCCGGCAACUUCACCGCCACCAUCGAA |
| | | | GAGUGGCAGGCCGAGCACGACGCCAUCAUGAGGCACAUCCUGGAGA |
| | | | GGCCCGACCCCACCGACGUGUUCCAGAACAAGGCCAACGUGUGCUG |
| | | | GGCCAAGGCCCUGGUGCCCGUGCUGAAGACCGCCGGCAUCGACAUG |
| | | | ACCACAGAGCAGUGGAACACCGUGGACUACUUCGAGACCGACAAGG |
| | | | CCCACAGCGCCGAGAUCGUGCUGAACCAGCUGUGCGUGAGGUUCUU |
| | | | CGGCCUGGACCUGGACAGCGGCCUGUUCAGCGCCCCCACCGUGCCA |
| | | | CUGAGCAUCAGGAACAACCACUGGGACAACAGCCCCAGCCCAAACA |
| | | | UGUACGGCCUGAACAAGGAGGUGGUCAGGCAGCUGAGCAGGCGGUA |
| | | | CCCACAGCUGCCCAGGGCCGUGGCCACCGGCAGGGUGUACGACAUG |
| | | | AACACCGGCACCCUGAGGAACUACGACCCCAGGAUCAACCUGGUGC |
| | | | CCGUGAACAGGCGGCUGCCCCACGCCCUGGUGCUGCACCACAACGA |
| | | | GCACCCACAGAGCGACUUCAGCUCCUUCGUGAGCAAGCUGAAAGGC |
| | | | AGGACCGUGCUGGUCGUGGGCGAGAAGCUGAGCGUGCCCGGCAAGA |
| | | | UGGUGGACUGGCUGAGCGACAGGCCCGAGGCCACCUUCCGGGCCAG |
| | | | GCUGGACCUCGGCAUCCCCGGCGACGUGCCCAAGUACGACAUCAUC |
| | | | UUCGUGAACGUCAGGACCCCAUACAAGUACCACCAUUACCAGCAGU |
| | | | GCGAGGACCACGCCAUCAAGCUGAGCAUGCUGACCAAGAAGGCCUG |
| | | | CCUGCACCUGAACCCCGGAGGCACCUGCGUGAGCAUCGGCUACGGC |
| | | | UACGCCGACAGGGCCAGCGAGAGCAUCAUUGGCGCCAUCGCCAGGC |
| | | | UGUUCAAGUUCAGCAGGGUGUGCAAACCCAAGAGCAGCCUGGAGGA |
| | | | AACCGAGGUGCUGUUCGUGUUCAUCGGCUACGACCGGAAGGCCAGG |
| | | | ACCCACAACCCCUACAAGCUGAGCAGCACCCUGACAAACAUCUACA |
| | | | CCGGCAGCAGGCUGCACGAGGCCGGCUGCGCCCCCAGCUACCACGU |
| | | | GGUCAGGGGCGAUAUCGCCACCGCCACCGAGGGCGUGAUCAUCAAC |
| | | | GCUGCCAACAGCAAGGGCCAGCCCGGAGGCGGAGUGUGCGGCGCCC |
| | | | UGUACAAGAAGUUCCCCGAGAGCUUCGACCUGCAGCCCAUCGAGGU |
| | | | GGGCAAGGCCAGGCUGGUGAAGGGCGCCGCUAAGCACAUCAUCCAC |
| | | | GCCGUGGGCCCCAACUUCAACAAGGUGAGCGAGGUGGAAGGCGACA |
| | | | AGCAGCUGGCCGAAGCCUACGAGAGCAUCGCCAAGAUCGUGAACGA |
| | | | CAAUAACUACAAGAGCGUGGCCAUCCCACUGCUCAGCACCGGCAUC |
| | | | UUCAGCGGCAACAAGGACAGGCUGACCCAGAGCCUGAACCACCUGC |
| | | | UCACCGCCCUGGACACCACCGAUGCCGACGUGGCCAUCUACUGCAG |
| | | | GGACAAGAAGUGGGAGAUGACCCUGAAGGAGGCCGUGGCCAGGCGG |
| | | | GAGGCCGUGGAAGAGAUCUGCAUCAGCGACGACUCCAGCGUGACCG |
| | | | AGCCCGACGCCGAGCUGGUGAGGGUGCACCCCAAGAGCUCCCUGGC |
| | | | CGGCAGGAAGGGCUACAGCACCAGCGACGGCAAGACCUUCAGCUAC |
| | | | CUGGAGGGCACCAAGUUCCACCAGGCCGCUAAGGACAUCGCCGAGA |
| | | | UCAACGCUAUGUGGCCCGUGGCCACCGAGGCCAACGAGCAGGUGUG |
| | | | CAUGUACAUCCUGGGCGAGAGCAUGUCCAGCAUCAGGAGCAAGUGC |
| | | | CCCGUGGAGGAAAGCGAGGCCAGCACACCACCCAGCACCCUGCCCU |
| | | | GCCUGUGCAUCCACGCUAUGACACCCGAGAGGGUGCAGCGGCUGAA |
| | | | GGCCAGCAGGCCCGAGCAGAUCACCGUGUGCAGCUCCUUCCCACUG |
| | | | CCCAAGUACAGGAUCACCGGCGUGCAGAAGAUCCAGUGCAGCCAGC |
| | | | CCAUCCUGUUCAGCCCAAAGGUGCCCGCCUACAUCCACCCCAGGAA |
| | | | GUACCUGGUGGAGACCCCACCCGUGGACGAGACACCCGAGCCAAGC |
| | | | GCCGAGAACCAGAGCACCGAGGGCACACCCGAGCAGCCACCCCUGA |
| | | | UCACCGAGGACGAGACAAGGACCCGGACCCCAGAGCCCAUCAUUAU |
| | | | CGAGGAAGAGGAAGAGGACAGCAUCAGCCUGCUGAGCGACGGCCCC |
| | | | ACCCACCAGGUGCUGCAGGUGGAGGCCGACAUCCACGGCCCACCCA |
| | | | GCGUGUCCAGCUCCAGCUGGAGCAUCCCACACGCCAGCGACUUCGA |
| | | | CGUGGACAGCCUGAGCAUCCUGGACACCCUGGAGGGCGCCAGCGUG |
| | | | ACCUCCGGCGCCACCAGCGCCGAGACCAACAGCUACUUCGCCAAGA |
| | | | GCAUGGAGUUCCUGGCCAGGCCCGUGCCAGCUCCCAGGACCGUGUU |
| | | | CAGGAACCCACCCCACCCAGCUCCCAGGACCAGGACCCCAAGCCUG |
| | | | GCUCCCAGCAGGGCCUGCAGCAGGACCAGCCUGGUGAGCACCCCAC |
| | | | CCGGCGUGAACAGGGUGAUCACCAGGGAGGAACUGGAGGCCCUGAC |
| | | | ACCCAGCAGGACCCCCAGCAGGUCCGUGAGCAGGACUAGUCUGGUG |
| | | | UCCAACCCACCCGGCGUGAACAGGGUGAUCACCAGGGAGGAAUUCG |
| | | | AGGCCUUCGUGGCCCAGCAACAGAGACGGUUCGACGCCGGCGCCUA |
| | | | CAUCUUCAGCAGCGACACCGGCCAGGGACACCUGCAGCAAAAGAGC |
| | | | GUGAGGCAGACCGUGCUGAGCGAGGUGGUGCUGGAGAGGACCGAGC |
| | | | UGGAAAUCAGCUACGCCCCCAGGCUGGACCAGGAGAAGGAGGAACU |
| | | | GCUCAGGAAGAAACUGCAGCUGAACCCCACCCCAGCCAACAGGAGC |
| | | | AGGUACCAGAGCAGGAAGGUGGAGAACAUGAAGGCCAUCACCGCCA |
| | | | GGCGGAUCCUGCAGGGCCUGGGACACUACCUGAAGGCCGAGGGCAA |
| | | | GGUGGAGUGCUACAGGACCCUGCACCCCGUGCCACUGUACAGCUCC |
| | | | AGCGUGAACAGGGCCUUCUCCAGCCCCAAGGUGGCCGUGGAGGCCU |
| | | | GCAACGCUAUGCUGAAGGAGAACUUCCCCACCGUGGCCAGCUACUG |
| | | | CAUCAUCCCCGAGUACGACGCCUACCUGGACAUGGUGGACGGCGCC |
| | | | AGCUGCUGCCUGGACACCGCCAGCUUCUGCCCCGCCAAGCUGAGGA |
| | | | GCUUCCCCAAGAAACACAGCUACCUGGAGCCCACCAUCAGGAGCGC |
| | | | CGUGCCCAGCGCCAUCCAGAACACCCUGCAGAACGUGCUGGCCGCU |
| | | | GCCACCAAGAGGAACUGCAACGUGACCCAGAUGAGGGAGCUGCCCG |
| | | | UGCUGGACAGCGCUGCCUUCAACGUGGAGUGCUUCAAGAAAUACGC |
| | | | CUGCAACAACGAGUACUGGGAGACCUUCAAGGAGAACCCCAUCAGG |
| | | | CUGACCGAAGAGAACGUGGUGAACUACAUCACCAAGCUGAAGGGCC |
| | | | CCAAGGCCGCUGCCCUGUUCGCUAAGACCCACAACCUGAACAUGCU |
| | | | GCAGGACAUCCCAAUGGACAGGUUCGUGAUGGACCUGAAGAGGGAC |
| | | | GUGAAGGUGACACCCGGCACCAAGCACACCGAGGAGAGGCCCAAGG |
| | | | UGCAGGUGAUCCAGGCCGCUGACCCACUGGCCACCGCCUACCUGUG |
| | | | CGGCAUCCACAGGGAGCUGGUGAGGCGGCUGAACGCCGUGCUGCUG |
| | | | CCCAACAUCCACACCCUGUUCGACAUGAGCGCCGAGGACUUCGACG |
| | | | CCAUCAUCGCCGAGCACUUCCAGCCCGGCGACUGCGUGCUGGAGAC |
| | | | CGACAUCGCCAGCUUCGACAAGAGCGAGGAUGACGCUAUGGCCCUG |
| | | | ACCGCUCUGAUGAUCCUGGAGGACCUGGGCGUGGACGCCGAGCUGC |
| | | | UCACCCUGAUCGAGGCUGCCUUCGGCGAGAUCAGCUCCAUCCACCU |
| | | | GCCCACCAAGACCAAGUUCAAGUUCGGCGCUAUGAUGAAAAGCGGA |
| | | | AUGUUCCUGACCCUGUUCGUGAACACCGUGAUCAACAUUGUGAUCG |
| | | | CCAGCAGGGUGCUGCGGGAGAGGCUGACCGGCAGCCCCUGCGCUGC |
| | | | CUUCAUCGGCGACGACAACAUCGUGAAGGGCGUGAAAAGCGACAAG |
| | | | CUGAUGGCCGACAGGUGCGCCACCUGGCUGAACAUGGAGGUGAAGA |
| | | | UCAUCGACGCCGUGGUGGGCGAGAAGGCCCCCUACUUCUGCGGCGG |
| | | | AUUCAUCCUGUGCGACAGCGUGACCGGCACCGCCUGCAGGGUGGCC |
| | | | GACCCCCUGAAGAGGCUGUUCAAGCUGGGCAAGCCACUGGCCGCUG |
| | | | ACGAUGAGCACGACGAUGACAGGCGGAGGGCCCUGCACGAGGAAAG |
| | | | CACCAGGUGGAACAGGGUGGGCAUCCUGAGCGAGCUGUGCAAGGCC |
| | | | GUGGAGAGCAGGUACGAGACCGUGGGCACCAGCAUCAUCGUGAUGG |
| | | | CUAUGACCACACUGGCCAGCUCCGUCAAGAGCUUCUCCUACCUGAG |
| | | | GGGGGCCCCUAUAACUCUCUACGGCUAACCUGAAUGGACUACGACA |
| | | | UAGUCUAGUCCGCCAAGGCCGCCACCAUGAGAGUGACAGCCCCUAG |
| | | | AACCUUACUGCUUCUGCUUUGGGGAGCUGUUGCUCUGACAGAGACA |
| | | | UGGGCUGGAUCUCUGAGCGAGGUGACCGGCCAGGGCCUGUGCAUCG |
| | | | GCGCCGUGCCCAAGACCCACCAGGUGCUGUGCAACACCACCCAGAA |
| | | | GACCAGCGACGGCAGCUACUACCUGGCCGCUCCCACCGGCACCACC |
| | | | UGGGCCUGCAGCACCGGCCUGACCCCUUGCAUCAGCACCACCAUCC |
| | | | UGAACCUGACCACCGACUACUGCGUGCUGGUGGAGCUGUGGCCCAG |
| | | | GGUGACCUACCACAGCCCCAGCUACGCCUACCACCAGUUCGAGAGG |
| | | | AGGGCCAAGUACAAGAGGGAGCCCGUGAGCCUGACCCUGGCCCUGC |
| | | | UGCUGGGCGGCCUGACAAUGGGCGGCAUCGCCGCCGGCGUGGGCAC |
| | | | CGGCACCACCGCCCUGGUGGCCACCCAGCAGUUCCAGCAGCUGCAG |
| | | | GCCGCCAUGCACGACGACCUGAAGGAGGUGGAGAAGUCCAUCACCA |
| | | | ACCUGGAGAAGUCCCUGACCAGCCUGAGCGAGGUGGUGCUGCAGAA |
| | | | CAGGAGGGGCCUGGACCUGCUGUUCCUGAAGGAGGGCGGCCUGUGC |
| | | | GCCGCCCUGAAGGAGGAGUGCUGCCUGUACGCCGACCACACCGGCC |
| | | | UGGUGAUCGUGGGCAUUGUCGCUGGCCUGGCCGUCCUCGCCGUGGU |
| | | | GGUGAUUGGAGCUGUGGUCGCAGCUGUUAUGUGCAGAAGAAAGUCA |
| | | | UCCGGCGGAAAGGGAGGCUCCUACUCUCAGGCUGCUUCUGCUACAG |
| | | | UGCCUAGAGCUCUUAUGUGUUUAUCUCAGCUGUAAACUCGAGUAUG |
| | | | UUACGUGCAAAGGUGAUUGUCACCCCCCGAAAGACCAUAUUGUGAC |
| | | | ACACCCUCAGUAUCACGCCCAAACAUUUACAGCCGCGGUGUCAAAA |
| | | | ACCGCGUGGACGUGGUUAACAUCCCUGCUGGGAGGAUCAGCCGUAA |
| | | | UUAUUAUAAUUGGCUUGGUGCUGGCUACUAUUGUGGCCAUGUACGU |
| | | | GCUGACCAACCAGAAACAUAAUUGAAUACAGCAGCAAUUGGCAAGC |
| | | | UGCUUACAUAGAACUCGCGGCGAUUGGCAUGCCGCCUUAAAAUUUU |
| | | | UAUUUUAUUUUUUCUUUUCUUUUCCGAAUCGGAUUUUGUUUUUAAU |
| | | | AUUUCAAAAAAAAAAAAAAAAAAAAAAAAAUCUAGAAAAAAAAAAA |
| | | | AAAAAAAAAAAAAAAAAAAAAAAAAAAAAAAAAAAAAAAAAAAAAA |
| | | | AAAAAAAAAAAAAAAAAAAAAAAAAAAAAAAAAAAAAAAAAAA |
| 3061 | STARR™ | 3061 | AUGGGCGGCGCAUGAGAGAAGCCCAGACCAAUUACCUACCCAAA |
| (SEQ | AH1A5 | | AUGGAGAAAGUUCACGUUGACAUCGAGGAAGACAGCCCAUUCCU |
| ID | | | CAGAGCUUUGCAGCGGAGCUUCCCGCAGUUUGAGGUAGAAGCCA |
| NO: | | | AGCAGGUCACUGAUAAUGACCAUGCUAAUGCCAGAGCGUUUUCG |
| 106) | | | CAUCUGGCUUCAAAACUGAUCGAAACGGAGGUGGACCCAUCCGA |
| | | | CACGAUCCUUGACAUUGGAAGUGCGCCCGCCCGCAGAAUGUAUU |
| | | | CUAAGCACAAGUAUCAUUGUAUCUGUCCGAUGAGAUGUGCGGAA |
| | | | GAUCCGGACAGAUUGUAUAAGUAUGCAACUAAGCUGAAGAAAAA |
| | | | CUGUAAGGAAAUAACUGAUAAGGAAUUGGACAAGAAAAUGAAGG |
| | | | AGCUGGCCGCCGUCAUGAGCGACCCUGACCUGGAAACUGAGACU |
| | | | AUGUGCCUCCACGACGACGAGUCGUGUCGCUACGAAGGGCAAGU |
| | | | CGCUGUUUACCAGGAUGUAUACGCCGUCGACGGCCCCACCAGCC |
| | | | UGUACCACCAGGCCAACAAGGGCGUGAGGGUGGCCUACUGGAUC |
| | | | GGCUUCGACACCACACCCUUCAUGUUCAAGAACCUGGCCGGCGC |
| | | | CUACCCCAGCUACAGCACCAACUGGGCCGACGAGACCGUGCUGA |
| | | | CCGCCAGGAACAUCGGCCUGUGCAGCAGCGACGUGAUGGAGAGG |
| | | | AGCCGGAGAGGCAUGAGCAUCCUGAGGAAGAAAUACCUGAAGCC |
| | | | CAGCAACAACGUGCUGUUCAGCGUGGGCAGCACCAUCUACCACG |
| | | | AGAAGAGGGACCUGCUCAGGAGCUGGCACCUGCCCAGCGUGUUC |
| | | | CACCUGAGGGGCAAGCAGAACUACACCUGCAGGUGCGAGACCAU |
| | | | CGUGAGCUGCGACGGCUACGUGGUGAAGAGGAUCGCCAUCAGCC |
| | | | CCGGCCUGUACGGCAAGCCCAGCGGCUACGCCGCUACAAUGCAC |
| | | | AGGGAGGGCUUCCUGUGCUGCAAGGUGACCGACACCCUGAACGG |
| | | | CGAGAGGGUGAGCUUCCCCGUGUGCACCUACGUGCCCGCCACCC |
| | | | UGUGCGACCAGAUGACCGGCAUCCUGGCCACCGACGUGAGCGCC |
| | | | GACGACGCCCAGAAGCUGCUCGUGGGCCUGAACCAGAGGAUCGU |
| | | | GGUCAACGGCAGGACCCAGAGGAACACCAACACAAUGAAGAACU |
| | | | ACCUGCUGCCCGUGGUGGCCCAGGCUUUCGCCAGGUGGGCCAAG |
| | | | GAGUACAAGGAGGACCAGGAAGACGAGAGGCCCCUGGGCCUGAG |
| | | | GGACAGGCAGCUGGUGAUGGGCUGCUGCUGGGCCUUCAGGCGGC |
| | | | ACAAGAUCACCAGCAUCUACAAGAGGCCCGACACCCAGACCAUC |
| | | | AUCAAGGUGAACAGCGACUUCCACAGCUUCGUGCUGCCCAGGAU |
| | | | CGGCAGCAACACCCUGGAGAUCGGCCUGAGGACCCGGAUCAGGA |
| | | | AGAUGCUGGAGGAACACAAGGAGCCCAGCCCACUGAUCACCGCC |
| | | | GAGGACGUGCAGGAGGCCAAGUGCGCUGCCGACGAGGCCAAGGA |
| | | | GGUGAGGGAGGCCGAGGAACUGAGGGCCGCCCUGCCACCCCUGG |
| | | | CUGCCGACGUGGAGGAACCCACCCUGGAAGCCGACGUGGACCUG |
| | | | AUGCUGCAGGAGGCCGGCGCCGGAAGCGUGGAGACACCCAGGGG |
| | | | CCUGAUCAAGGUGACCAGCUACGACGGCGAGGACAAGAUCGGCA |
| | | | GCUACGCCGUGCUGAGCCCACAGGCCGUGCUGAAGUCCGAGAAG |
| | | | CUGAGCUGCAUCCACCCACUGGCCGAGCAGGUGAUCGUGAUCAC |
| | | | CCACAGCGGCAGGAAGGGCAGGUACGCCGUGGAGCCCUACCACG |
| | | | GCAAGGUGGUCGUGCCCGAGGGCCACGCCAUCCCCGUGCAGGAC |
| | | | UUCCAGGCCCUGAGCGAGAGCGCCACCAUCGUGUACAACGAGAG |
| | | | GGAGUUCGUGAACAGGUACCUGCACCAUAUCGCCACCCACGGCG |
| | | | GAGCCCUGAACACCGACGAGGAAUACUACAAGACCGUGAAGCCC |
| | | | AGCGAGCACGACGGCGAGUACCUGUACGACAUCGACAGGAAGCA |
| | | | GUGCGUGAAGAAAGAGCUGGUGACCGGCCUGGGACUGACCGGCG |
| | | | AGCUGGUGGACCCACCCUUCCACGAGUUCGCCUACGAGAGCCUG |
| | | | AGGACCAGACCCGCCGCUCCCUACCAGGUGCCCACCAUCGGCGU |
| | | | GUACGGCGUGCCCGGCAGCGGAAAGAGCGGCAUCAUCAAGAGCG |
| | | | CCGUGACCAAGAAAGACCUGGUGGUCAGCGCCAAGAAAGAGAAC |
| | | | UGCGCCGAGAUCAUCAGGGACGUGAAGAAGAUGAAAGGCCUGGA |
| | | | CGUGAACGCGCGCACCGUGGACAGCGUGCUGCUGAACGGCUGCA |
| | | | AGCACCCCGUGGAGACCCUGUACAUCGACGAGGCCUUCGCUUGC |
| | | | CACGCCGGCACCCUGAGGGCCCUGAUCGCCAUCAUCAGGCCCAA |
| | | | GAAAGCCGUGCUGUGCGGCGACCCCAAGCAGUGCGGCUUCUUCA |
| | | | ACAUGAUGUGCCUGAAGGUGCACUUCAACCACGAGAUCUGCACC |
| | | | CAGGUGUUCCACAAGAGCAUCAGCAGGCGGUGCACCAAGAGCGU |
| | | | GACCAGCGUCGUGAGCACCCUGUUCUACGACAAGAAAAUGAGGA |
| | | | CCACCAACCCCAAGGAGACCAAAAUCGUGAUCGACACCACAGGC |
| | | | AGCACCAAGCCCAAGCAGGACGACCUGAUCCUGACCUGCUUCAG |
| | | | GGGCUGGGUGAAGCAGCUGCAGAUCGACUACAAGGGCAACGAGA |
| | | | UCAUGACCGCCGCUGCCAGCCAGGGCCUGACCAGGAAGGGCGUG |
| | | | UACGCCGUGAGGUACAAGGUGAACGAGAACCCACUGUACGCUCC |
| | | | CACCAGCGAGCACGUGAACGUGCUGCUGACCAGGACCGAGGACA |
| | | | GGAUCGUGUGGAAGACCCUGGCCGGCGACCCCUGGAUCAAGACC |
| | | | CUGACCGCCAAGUACCCCGGCAACUUCACCGCCACCAUCGAAGA |
| | | | GUGGCAGGCCGAGCACGACGCCAUCAUGAGGCACAUCCUGGAGA |
| | | | GGCCCGACCCCACCGACGUGUUCCAGAACAAGGCCAACGUGUGC |
| | | | UGGGCCAAGGCCCUGGUGCCCGUGCUGAAGACCGCCGGCAUCGA |
| | | | CAUGACCACAGAGCAGUGGAACACCGUGGACUACUUCGAGACCG |
| | | | ACAAGGCCCACAGCGCCGAGAUCGUGCUGAACCAGCUGUGCGUG |
| | | | AGGUUCUUCGGCCUGGACCUGGACAGCGGCCUGUUCAGCGCCCC |
| | | | CACCGUGCCACUGAGCAUCAGGAACAACCACUGGGACAACAGCC |
| | | | CCAGCCCAAACAUGUACGGCCUGAACAAGGAGGUGGUCAGGCAG |
| | | | CUGAGCAGGCGGUACCCACAGCUGCCCAGGGCCGUGGCCACCGG |
| | | | CAGGGUGUACGACAUGAACACCGGCACCCUGAGGAACUACGACC |
| | | | CCAGGAUCAACCUGGUGCCCGUGAACAGGCGGCUGCCCCACGCC |
| | | | CUGGUGCUGCACCACAACGAGCACCCACAGAGCGACUUCAGCUC |
| | | | CUUCGUGAGCAAGCUGAAAGGCAGGACCGUGCUGGUCGUGGGCG |
| | | | AGAAGCUGAGCGUGCCCGGCAAGAUGGUGGACUGGCUGAGCGAC |
| | | | AGGCCCGAGGCCACCUUCCGGGCCAGGCUGGACCUCGGCAUCCC |
| | | | CGGCGACGUGCCCAAGUACGACAUCAUCUUCGUGAACGUCAGGA |
| | | | CCCCAUACAAGUACCACCAUUACCAGCAGUGCGAGGACCACGCC |
| | | | AUCAAGCUGAGCAUGCUGACCAAGAAGGCCUGCCUGCACCUGAA |
| | | | CCCCGGAGGCACCUGCGUGAGCAUCGGCUACGGCUACGCCGACA |
| | | | GGGCCAGCGAGAGCAUCAUUGGCGCCAUCGCCAGGCUGUUCAAG |
| | | | UUCAGCAGGGUGUGCAAACCCAAGAGCAGCCUGGAGGAAACCGA |
| | | | GGUGCUGUUCGUGUUCAUCGGCUACGACCGGAAGGCCAGGACCC |
| | | | ACAACCCCUACAAGCUGAGCAGCACCCUGACAAACAUCUACACC |
| | | | GGCAGCAGGCUGCACGAGGCCGGCUGCGCCCCCAGCUACCACGU |
| | | | GGUCAGGGGCGAUAUCGCCACCGCCACCGAGGGCGUGAUCAUCA |
| | | | ACGCUGCCAACAGCAAGGGCCAGCCCGGAGGCGGAGUGUGCGGC |
| | | | GCCCUGUACAAGAAGUUCCCCGAGAGCUUCGACCUGCAGCCCAU |
| | | | CGAGGUGGGCAAGGCCAGGCUGGUGAAGGGCGCCGCUAAGCACA |
| | | | UCAUCCACGCCGUGGGCCCCAACUUCAACAAGGUGAGCGAGGUG |
| | | | GAAGGCGACAAGCAGCUGGCCGAAGCCUACGAGAGCAUCGCCAA |
| | | | GAUCGUGAACGACAAUAACUACAAGAGCGUGGCCAUCCCACUGC |
| | | | UCAGCACCGGCAUCUUCAGCGGCAACAAGGACAGGCUGACCCAG |
| | | | AGCCUGAACCACCUGCUCACCGCCCUGGACACCACCGAUGCCGA |
| | | | CGUGGCCAUCUACUGCAGGGACAAGAAGUGGGAGAUGACCCUGA |
| | | | AGGAGGCCGUGGCCAGGCGGGAGGCCGUGGAAGAGAUCUGCAUC |
| | | | AGCGACGACUCCAGCGUGACCGAGCCCGACGCCGAGCUGGUGAG |
| | | | GGUGCACCCCAAGAGCUCCCUGGCCGGCAGGAAGGGCUACAGCA |
| | | | CCAGCGACGGCAAGACCUUCAGCUACCUGGAGGGCACCAAGUUC |
| | | | CACCAGGCCGCUAAGGACAUCGCCGAGAUCAACGCUAUGUGGCC |
| | | | CGUGGCCACCGAGGCCAACGAGCAGGUGUGCAUGUACAUCCUGG |
| | | | GCGAGAGCAUGUCCAGCAUCAGGAGCAAGUGCCCCGUGGAGGAA |
| | | | AGCGAGGCCAGCACACCACCCAGCACCCUGCCCUGCCUGUGCAU |
| | | | CCACGCUAUGACACCCGAGAGGGUGCAGCGGCUGAAGGCCAGCA |
| | | | GGCCCGAGCAGAUCACCGUGUGCAGCUCCUUCCCACUGCCCAAG |
| | | | UACAGGAUCACCGGCGUGCAGAAGAUCCAGUGCAGCCAGCCCAU |
| | | | CCUGUUCAGCCCAAAGGUGCCCGCCUACAUCCACCCCAGGAAGU |
| | | | ACCUGGUGGAGACCCCACCCGUGGACGAGACACCCGAGCCAAGC |
| | | | GCCGAGAACCAGAGCACCGAGGGCACACCCGAGCAGCCACCCCU |
| | | | GAUCACCGAGGACGAGACAAGGACCCGGACCCCAGAGCCCAUCA |
| | | | UUAUCGAGGAAGAGGAAGAGGACAGCAUCAGCCUGCUGAGCGAC |
| | | | GGCCCCACCCACCAGGUGCUGCAGGUGGAGGCCGACAUCCACGG |
| | | | CCCACCCAGCGUGUCCAGCUCCAGCUGGAGCAUCCCACACGCCA |
| | | | GCGACUUCGACGUGGACAGCCUGAGCAUCCUGGACACCCUGGAG |
| | | | GGCGCCAGCGUGACCUCCGGCGCCACCAGCGCCGAGACCAACAG |
| | | | CUACUUCGCCAAGAGCAUGGAGUUCCUGGCCAGGCCCGUGCCAG |
| | | | CUCCCAGGACCGUGUUCAGGAACCCACCCCACCCAGCUCCCAGG |
| | | | ACCAGGACCCCAAGCCUGGCUCCCAGCAGGGCCUGCAGCAGGAC |
| | | | CAGCCUGGUGAGCACCCCACCCGGCGUGAACAGGGUGAUCACCA |
| | | | GGGAGGAACUGGAGGCCCUGACACCCAGCAGGACCCCCAGCAGG |
| | | | UCCGUGAGCAGGACUAGUCUGGUGUCCAACCCACCCGGCGUGAA |
| | | | CAGGGUGAUCACCAGGGAGGAAUUCGAGGCCUUCGUGGCCCAGC |
| | | | AACAGAGACGGUUCGACGCCGGCGCCUACAUCUUCAGCAGCGAC |
| | | | ACCGGCCAGGGACACCUGCAGCAAAAGAGCGUGAGGCAGACCGU |
| | | | GCUGAGCGAGGUGGUGCUGGAGAGGACCGAGCUGGAAAUCAGCU |
| | | | ACGCCCCCAGGCUGGACCAGGAGAAGGAGGAACUGCUCAGGAAG |
| | | | AAACUGCAGCUGAACCCCACCCCAGCCAACAGGAGCAGGUACCA |
| | | | GAGCAGGAAGGUGGAGAACAUGAAGGCCAUCACCGCCAGGCGGA |
| | | | UCCUGCAGGGCCUGGGACACUACCUGAAGGCCGAGGGCAAGGUG |
| | | | GAGUGCUACAGGACCCUGCACCCCGUGCCACUGUACAGCUCCAG |
| | | | CGUGAACAGGGCCUUCUCCAGCCCCAAGGUGGCCGUGGAGGCCU |
| | | | GCAACGCUAUGCUGAAGGAGAACUUCCCCACCGUGGCCAGCUAC |
| | | | UGCAUCAUCCCCGAGUACGACGCCUACCUGGACAUGGUGGACGG |
| | | | CGCCAGCUGCUGCCUGGACACCGCCAGCUUCUGCCCCGCCAAGC |
| | | | UGAGGAGCUUCCCCAAGAAACACAGCUACCUGGAGCCCACCAUC |
| | | | AGGAGCGCCGUGCCCAGCGCCAUCCAGAACACCCUGCAGAACGU |
| | | | GCUGGCCGCUGCCACCAAGAGGAACUGCAACGUGACCCAGAUGA |
| | | | GGGAGCUGCCCGUGCUGGACAGCGCUGCCUUCAACGUGGAGUGC |
| | | | UUCAAGAAAUACGCCUGCAACAACGAGUACUGGGAGACCUUCAA |
| | | | GGAGAACCCCAUCAGGCUGACCGAAGAGAACGUGGUGAACUACA |
| | | | UCACCAAGCUGAAGGGCCCCAAGGCCGCUGCCCUGUUCGCUAAG |
| | | | ACCCACAACCUGAACAUGCUGCAGGACAUCCCAAUGGACAGGUU |
| | | | CGUGAUGGACCUGAAGAGGGACGUGAAGGUGACACCCGGCACCA |
| | | | AGCACACCGAGGAGAGGCCCAAGGUGCAGGUGAUCCAGGCCGCU |
| | | | GACCCACUGGCCACCGCCUACCUGUGCGGCAUCCACAGGGAGCU |
| | | | GGUGAGGCGGCUGAACGCCGUGCUGCUGCCCAACAUCCACACCC |
| | | | UGUUCGACAUGAGCGCCGAGGACUUCGACGCCAUCAUCGCCGAG |
| | | | CACUUCCAGCCCGGCGACUGCGUGCUGGAGACCGACAUCGCCAG |
| | | | CUUCGACAAGAGCGAGGAUGACGCUAUGGCCCUGACCGCUCUGA |
| | | | UGAUCCUGGAGGACCUGGGCGUGGACGCCGAGCUGCUCACCCUG |
| | | | AUCGAGGCUGCCUUCGGCGAGAUCAGCUCCAUCCACCUGCCCAC |
| | | | CAAGACCAAGUUCAAGUUCGGCGCUAUGAUGAAAAGCGGAAUGU |
| | | | UCCUGACCCUGUUCGUGAACACCGUGAUCAACAUUGUGAUCGCC |
| | | | AGCAGGGUGCUGCGGGAGAGGCUGACCGGCAGCCCCUGCGCUGC |
| | | | CUUCAUCGGCGACGACAACAUCGUGAAGGGCGUGAAAAGCGACA |
| | | | AGCUGAUGGCCGACAGGUGCGCCACCUGGCUGAACAUGGAGGUG |
| | | | AAGAUCAUCGACGCCGUGGUGGGCGAGAAGGCCCCCUACUUCUG |
| | | | CGGCGGAUUCAUCCUGUGCGACAGCGUGACCGGCACCGCCUGCA |
| | | | GGGUGGCCGACCCCCUGAAGAGGCUGUUCAAGCUGGGCAAGCCA |
| | | | CUGGCCGCUGACGAUGAGCACGACGAUGACAGGCGGAGGGCCCU |
| | | | GCACGAGGAAAGCACCAGGUGGAACAGGGUGGGCAUCCUGAGCG |
| | | | AGCUGUGCAAGGCCGUGGAGAGCAGGUACGAGACCGUGGGCACC |
| | | | AGCAUCAUCGUGAUGGCUAUGACCACACUGGCCAGCUCCGUCAA |
| | | | GAGCUUCUCCUACCUGAGGGGGGCCCCUAUAACUCUCUACGGCU |
| | | | AACCUGAAUGGACUACGACAUAGUCUAGUCCGCCAAGGCCGCCA |
| | | | CCAUGAGAGUGACAGCCCCUAGAACCUUACUGCUUCUGCUUUGG |
| | | | GGAGCUGUUGCUCUGACAGAGACAUGGGCUGGAUCUUACCACAG |
| | | | CCCCAGCUACGCCUACCACCAGUUCGAGAGGGGGGGAGGAGGCU |
| | | | CCGGGGGAGGAGGCUCCCUGAAGAUCAGCCAGGCCGUGCACGCC |
| | | | GCCCACGCCGAGAUCAACGAGGCCGGCCGGGAGGUGAUCGUGGG |
| | | | CAUUGUCGCUGGCCUGGCCGUCCUCGCCGUGGUGGUGAUUGGAG |
| | | | CUGUGGUCGCAGCUGUUAUGUGCAGAAGAAAGUCAUCCGGCGGA |
| | | | AAGGGAGGCUCCUACUCUCAGGCUGCUUCUGCUACAGUGCCUAG |
| | | | AGCUCUUAUGUGUUUAUCUCAGCUGUAAACUCGAGUAUGUUACG |
| | | | UGCAAAGGUGAUUGUCACCCCCCGAAAGACCAUAUUGUGACACA |
| | | | CCCUCAGUAUCACGCCCAAACAUUUACAGCCGCGGUGUCAAAAA |
| | | | CCGCGUGGACGUGGUUAACAUCCCUGCUGGGAGGAUCAGCCGUA |
| | | | AUUAUUAUAAUUGGCUUGGUGCUGGCUACUAUUGUGGCCAUGUA |
| | | | CGUGCUGACCAACCAGAAACAUAAUUGAAUACAGCAGCAAUUGG |
| | | | CAAGCUGCUUACAUAGAACUCGCGGCGAUUGGCAUGCCGCCUUA |
| | | | AAAUUUUUAUUUUAUUUUUUCUUUUCUUUUCCGAAUCGGAUUUU |
| | | | GUUUUUAAUAUUUCAAAAAAAAAAAAAAAAAAAAAAAAAUCUAG |
| | | | AAAAAAAAAAAAAAAAAAAAAAAAAAAAAAAAAAAAAAAAAAAA |
| | | | AAAAAAAAAAAAAAAAAAAAAAAAAAAAAAAAAAAAAAAAAAAA |
| | | | AAAAAAAAAAAA |
| 3067 | STARR™ | 3067 | AUGGGCGGCGCAUGAGAGAAGCCCAGACCAAUUACCUACCCAAAAU |
| (SEQ | gp70- | | GGAGAAAGUUCACGUUGACAUCGAGGAAGACAGCCCAUUCCUCAGA |
| ID | FLAG | | GCUUUGCAGCGGAGCUUCCCGCAGUUUGAGGUAGAAGCCAAGCAGG |
| NO: | | | UCACUGAUAAUGACCAUGCUAAUGCCAGAGCGUUUUCGCAUCUGGC |
| 107) | | | UUCAAAACUGAUCGAAACGGAGGUGGACCCAUCCGACACGAUCCUU |
| | | | GACAUUGGAAGUGCGCCCGCCCGCAGAAUGUAUUCUAAGCACAAGU |
| | | | AUCAUUGUAUCUGUCCGAUGAGAUGUGCGGAAGAUCCGGACAGAUU |
| | | | GUAUAAGUAUGCAACUAAGCUGAAGAAAAACUGUAAGGAAAUAACU |
| | | | GAUAAGGAAUUGGACAAGAAAAUGAAGGAGCUGGCCGCCGUCAUGA |
| | | | GCGACCCUGACCUGGAAACUGAGACUAUGUGCCUCCACGACGACGA |
| | | | GUCGUGUCGCUACGAAGGGCAAGUCGCUGUUUACCAGGAUGUAUAC |
| | | | GCCGUCGACGGCCCCACCAGCCUGUACCACCAGGCCAACAAGGGCG |
| | | | UGAGGGUGGCCUACUGGAUCGGCUUCGACACCACACCCUUCAUGUU |
| | | | CAAGAACCUGGCCGGCGCCUACCCCAGCUACAGCACCAACUGGGCC |
| | | | GACGAGACCGUGCUGACCGCCAGGAACAUCGGCCUGUGCAGCAGCG |
| | | | ACGUGAUGGAGAGGAGCCGGAGAGGCAUGAGCAUCCUGAGGAAGAA |
| | | | AUACCUGAAGCCCAGCAACAACGUGCUGUUCAGCGUGGGCAGCACC |
| | | | AUCUACCACGAGAAGAGGGACCUGCUCAGGAGCUGGCACCUGCCCA |
| | | | GCGUGUUCCACCUGAGGGGCAAGCAGAACUACACCUGCAGGUGCGA |
| | | | GACCAUCGUGAGCUGCGACGGCUACGUGGUGAAGAGGAUCGCCAUC |
| | | | AGCCCCGGCCUGUACGGCAAGCCCAGCGGCUACGCCGCUACAAUGC |
| | | | ACAGGGAGGGCUUCCUGUGCUGCAAGGUGACCGACACCCUGAACGG |
| | | | CGAGAGGGUGAGCUUCCCCGUGUGCACCUACGUGCCCGCCACCCUG |
| | | | UGCGACCAGAUGACCGGCAUCCUGGCCACCGACGUGAGCGCCGACG |
| | | | ACGCCCAGAAGCUGCUCGUGGGCCUGAACCAGAGGAUCGUGGUCAA |
| | | | CGGCAGGACCCAGAGGAACACCAACACAAUGAAGAACUACCUGCUG |
| | | | CCCGUGGUGGCCCAGGCUUUCGCCAGGUGGGCCAAGGAGUACAAGG |
| | | | AGGACCAGGAAGACGAGAGGCCCCUGGGCCUGAGGGACAGGCAGCU |
| | | | GGUGAUGGGCUGCUGCUGGGCCUUCAGGCGGCACAAGAUCACCAGC |
| | | | AUCUACAAGAGGCCCGACACCCAGACCAUCAUCAAGGUGAACAGCG |
| | | | ACUUCCACAGCUUCGUGCUGCCCAGGAUCGGCAGCAACACCCUGGA |
| | | | GAUCGGCCUGAGGACCCGGAUCAGGAAGAUGCUGGAGGAACACAAG |
| | | | GAGCCCAGCCCACUGAUCACCGCCGAGGACGUGCAGGAGGCCAAGU |
| | | | GCGCUGCCGACGAGGCCAAGGAGGUGAGGGAGGCCGAGGAACUGAG |
| | | | GGCCGCCCUGCCACCCCUGGCUGCCGACGUGGAGGAACCCACCCUG |
| | | | GAAGCCGACGUGGACCUGAUGCUGCAGGAGGCCGGCGCCGGAAGCG |
| | | | UGGAGACACCCAGGGGCCUGAUCAAGGUGACCAGCUACGACGGCGA |
| | | | GGACAAGAUCGGCAGCUACGCCGUGCUGAGCCCACAGGCCGUGCUG |
| | | | AAGUCCGAGAAGCUGAGCUGCAUCCACCCACUGGCCGAGCAGGUGA |
| | | | UCGUGAUCACCCACAGCGGCAGGAAGGGCAGGUACGCCGUGGAGCC |
| | | | CUACCACGGCAAGGUGGUCGUGCCCGAGGGCCACGCCAUCCCCGUG |
| | | | CAGGACUUCCAGGCCCUGAGCGAGAGCGCCACCAUCGUGUACAACG |
| | | | AGAGGGAGUUCGUGAACAGGUACCUGCACCAUAUCGCCACCCACGG |
| | | | CGGAGCCCUGAACACCGACGAGGAAUACUACAAGACCGUGAAGCCC |
| | | | AGCGAGCACGACGGCGAGUACCUGUACGACAUCGACAGGAAGCAGU |
| | | | GCGUGAAGAAAGAGCUGGUGACCGGCCUGGGACUGACCGGCGAGCU |
| | | | GGUGGACCCACCCUUCCACGAGUUCGCCUACGAGAGCCUGAGGACC |
| | | | AGACCCGCCGCUCCCUACCAGGUGCCCACCAUCGGCGUGUACGGCG |
| | | | UGCCCGGCAGCGGAAAGAGCGGCAUCAUCAAGAGCGCCGUGACCAA |
| | | | GAAAGACCUGGUGGUCAGCGCCAAGAAAGAGAACUGCGCCGAGAUC |
| | | | AUCAGGGACGUGAAGAAGAUGAAAGGCCUGGACGUGAACGCGCGCA |
| | | | CCGUGGACAGCGUGCUGCUGAACGGCUGCAAGCACCCCGUGGAGAC |
| | | | CCUGUACAUCGACGAGGCCUUCGCUUGCCACGCCGGCACCCUGAGG |
| | | | GCCCUGAUCGCCAUCAUCAGGCCCAAGAAAGCCGUGCUGUGCGGCG |
| | | | ACCCCAAGCAGUGCGGCUUCUUCAACAUGAUGUGCCUGAAGGUGCA |
| | | | CUUCAACCACGAGAUCUGCACCCAGGUGUUCCACAAGAGCAUCAGC |
| | | | AGGCGGUGCACCAAGAGCGUGACCAGCGUCGUGAGCACCCUGUUCU |
| | | | ACGACAAGAAAAUGAGGACCACCAACCCCAAGGAGACCAAAAUCGU |
| | | | GAUCGACACCACAGGCAGCACCAAGCCCAAGCAGGACGACCUGAUC |
| | | | CUGACCUGCUUCAGGGGCUGGGUGAAGCAGCUGCAGAUCGACUACA |
| | | | AGGGCAACGAGAUCAUGACCGCCGCUGCCAGCCAGGGCCUGACCAG |
| | | | GAAGGGCGUGUACGCCGUGAGGUACAAGGUGAACGAGAACCCACUG |
| | | | UACGCUCCCACCAGCGAGCACGUGAACGUGCUGCUGACCAGGACCG |
| | | | AGGACAGGAUCGUGUGGAAGACCCUGGCCGGCGACCCCUGGAUCAA |
| | | | GACCCUGACCGCCAAGUACCCCGGCAACUUCACCGCCACCAUCGAA |
| | | | GAGUGGCAGGCCGAGCACGACGCCAUCAUGAGGCACAUCCUGGAGA |
| | | | GGCCCGACCCCACCGACGUGUUCCAGAACAAGGCCAACGUGUGCUG |
| | | | GGCCAAGGCCCUGGUGCCCGUGCUGAAGACCGCCGGCAUCGACAUG |
| | | | ACCACAGAGCAGUGGAACACCGUGGACUACUUCGAGACCGACAAGG |
| | | | CCCACAGCGCCGAGAUCGUGCUGAACCAGCUGUGCGUGAGGUUCUU |
| | | | CGGCCUGGACCUGGACAGCGGCCUGUUCAGCGCCCCCACCGUGCCA |
| | | | CUGAGCAUCAGGAACAACCACUGGGACAACAGCCCCAGCCCAAACA |
| | | | UGUACGGCCUGAACAAGGAGGUGGUCAGGCAGCUGAGCAGGCGGUA |
| | | | CCCACAGCUGCCCAGGGCCGUGGCCACCGGCAGGGUGUACGACAUG |
| | | | AACACCGGCACCCUGAGGAACUACGACCCCAGGAUCAACCUGGUGC |
| | | | CCGUGAACAGGCGGCUGCCCCACGCCCUGGUGCUGCACCACAACGA |
| | | | GCACCCACAGAGCGACUUCAGCUCCUUCGUGAGCAAGCUGAAAGGC |
| | | | AGGACCGUGCUGGUCGUGGGCGAGAAGCUGAGCGUGCCCGGCAAGA |
| | | | UGGUGGACUGGCUGAGCGACAGGCCCGAGGCCACCUUCCGGGCCAG |
| | | | GCUGGACCUCGGCAUCCCCGGCGACGUGCCCAAGUACGACAUCAUC |
| | | | UUCGUGAACGUCAGGACCCCAUACAAGUACCACCAUUACCAGCAGU |
| | | | GCGAGGACCACGCCAUCAAGCUGAGCAUGCUGACCAAGAAGGCCUG |
| | | | CCUGCACCUGAACCCCGGAGGCACCUGCGUGAGCAUCGGCUACGGC |
| | | | UACGCCGACAGGGCCAGCGAGAGCAUCAUUGGCGCCAUCGCCAGGC |
| | | | UGUUCAAGUUCAGCAGGGUGUGCAAACCCAAGAGCAGCCUGGAGGA |
| | | | AACCGAGGUGCUGUUCGUGUUCAUCGGCUACGACCGGAAGGCCAGG |
| | | | ACCCACAACCCCUACAAGCUGAGCAGCACCCUGACAAACAUCUACA |
| | | | CCGGCAGCAGGCUGCACGAGGCCGGCUGCGCCCCCAGCUACCACGU |
| | | | GGUCAGGGGCGAUAUCGCCACCGCCACCGAGGGCGUGAUCAUCAAC |
| | | | GCUGCCAACAGCAAGGGCCAGCCCGGAGGCGGAGUGUGCGGCGCCC |
| | | | UGUACAAGAAGUUCCCCGAGAGCUUCGACCUGCAGCCCAUCGAGGU |
| | | | GGGCAAGGCCAGGCUGGUGAAGGGCGCCGCUAAGCACAUCAUCCAC |
| | | | GCCGUGGGCCCCAACUUCAACAAGGUGAGCGAGGUGGAAGGCGACA |
| | | | AGCAGCUGGCCGAAGCCUACGAGAGCAUCGCCAAGAUCGUGAACGA |
| | | | CAAUAACUACAAGAGCGUGGCCAUCCCACUGCUCAGCACCGGCAUC |
| | | | UUCAGCGGCAACAAGGACAGGCUGACCCAGAGCCUGAACCACCUGC |
| | | | UCACCGCCCUGGACACCACCGAUGCCGACGUGGCCAUCUACUGCAG |
| | | | GGACAAGAAGUGGGAGAUGACCCUGAAGGAGGCCGUGGCCAGGCGG |
| | | | GAGGCCGUGGAAGAGAUCUGCAUCAGCGACGACUCCAGCGUGACCG |
| | | | AGCCCGACGCCGAGCUGGUGAGGGUGCACCCCAAGAGCUCCCUGGC |
| | | | CGGCAGGAAGGGCUACAGCACCAGCGACGGCAAGACCUUCAGCUAC |
| | | | CUGGAGGGCACCAAGUUCCACCAGGCCGCUAAGGACAUCGCCGAGA |
| | | | UCAACGCUAUGUGGCCCGUGGCCACCGAGGCCAACGAGCAGGUGUG |
| | | | CAUGUACAUCCUGGGCGAGAGCAUGUCCAGCAUCAGGAGCAAGUGC |
| | | | CCCGUGGAGGAAAGCGAGGCCAGCACACCACCCAGCACCCUGCCCU |
| | | | GCCUGUGCAUCCACGCUAUGACACCCGAGAGGGUGCAGCGGCUGAA |
| | | | GGCCAGCAGGCCCGAGCAGAUCACCGUGUGCAGCUCCUUCCCACUG |
| | | | CCCAAGUACAGGAUCACCGGCGUGCAGAAGAUCCAGUGCAGCCAGC |
| | | | CCAUCCUGUUCAGCCCAAAGGUGCCCGCCUACAUCCACCCCAGGAA |
| | | | GUACCUGGUGGAGACCCCACCCGUGGACGAGACACCCGAGCCAAGC |
| | | | GCCGAGAACCAGAGCACCGAGGGCACACCCGAGCAGCCACCCCUGA |
| | | | UCACCGAGGACGAGACAAGGACCCGGACCCCAGAGCCCAUCAUUAU |
| | | | CGAGGAAGAGGAAGAGGACAGCAUCAGCCUGCUGAGCGACGGCCCC |
| | | | ACCCACCAGGUGCUGCAGGUGGAGGCCGACAUCCACGGCCCACCCA |
| | | | GCGUGUCCAGCUCCAGCUGGAGCAUCCCACACGCCAGCGACUUCGA |
| | | | CGUGGACAGCCUGAGCAUCCUGGACACCCUGGAGGGCGCCAGCGUG |
| | | | ACCUCCGGCGCCACCAGCGCCGAGACCAACAGCUACUUCGCCAAGA |
| | | | GCAUGGAGUUCCUGGCCAGGCCCGUGCCAGCUCCCAGGACCGUGUU |
| | | | CAGGAACCCACCCCACCCAGCUCCCAGGACCAGGACCCCAAGCCUG |
| | | | GCUCCCAGCAGGGCCUGCAGCAGGACCAGCCUGGUGAGCACCCCAC |
| | | | CCGGCGUGAACAGGGUGAUCACCAGGGAGGAACUGGAGGCCCUGAC |
| | | | ACCCAGCAGGACCCCCAGCAGGUCCGUGAGCAGGACUAGUCUGGUG |
| | | | UCCAACCCACCCGGCGUGAACAGGGUGAUCACCAGGGAGGAAUUCG |
| | | | AGGCCUUCGUGGCCCAGCAACAGAGACGGUUCGACGCCGGCGCCUA |
| | | | CAUCUUCAGCAGCGACACCGGCCAGGGACACCUGCAGCAAAAGAGC |
| | | | GUGAGGCAGACCGUGCUGAGCGAGGUGGUGCUGGAGAGGACCGAGC |
| | | | UGGAAAUCAGCUACGCCCCCAGGCUGGACCAGGAGAAGGAGGAACU |
| | | | GCUCAGGAAGAAACUGCAGCUGAACCCCACCCCAGCCAACAGGAGC |
| | | | AGGUACCAGAGCAGGAAGGUGGAGAACAUGAAGGCCAUCACCGCCA |
| | | | GGCGGAUCCUGCAGGGCCUGGGACACUACCUGAAGGCCGAGGGCAA |
| | | | GGUGGAGUGCUACAGGACCCUGCACCCCGUGCCACUGUACAGCUCC |
| | | | AGCGUGAACAGGGCCUUCUCCAGCCCCAAGGUGGCCGUGGAGGCCU |
| | | | GCAACGCUAUGCUGAAGGAGAACUUCCCCACCGUGGCCAGCUACUG |
| | | | CAUCAUCCCCGAGUACGACGCCUACCUGGACAUGGUGGACGGCGCC |
| | | | AGCUGCUGCCUGGACACCGCCAGCUUCUGCCCCGCCAAGCUGAGGA |
| | | | GCUUCCCCAAGAAACACAGCUACCUGGAGCCCACCAUCAGGAGCGC |
| | | | CGUGCCCAGCGCCAUCCAGAACACCCUGCAGAACGUGCUGGCCGCU |
| | | | GCCACCAAGAGGAACUGCAACGUGACCCAGAUGAGGGAGCUGCCCG |
| | | | UGCUGGACAGCGCUGCCUUCAACGUGGAGUGCUUCAAGAAAUACGC |
| | | | CUGCAACAACGAGUACUGGGAGACCUUCAAGGAGAACCCCAUCAGG |
| | | | CUGACCGAAGAGAACGUGGUGAACUACAUCACCAAGCUGAAGGGCC |
| | | | CCAAGGCCGCUGCCCUGUUCGCUAAGACCCACAACCUGAACAUGCU |
| | | | GCAGGACAUCCCAAUGGACAGGUUCGUGAUGGACCUGAAGAGGGAC |
| | | | GUGAAGGUGACACCCGGCACCAAGCACACCGAGGAGAGGCCCAAGG |
| | | | UGCAGGUGAUCCAGGCCGCUGACCCACUGGCCACCGCCUACCUGUG |
| | | | CGGCAUCCACAGGGAGCUGGUGAGGCGGCUGAACGCCGUGCUGCUG |
| | | | CCCAACAUCCACACCCUGUUCGACAUGAGCGCCGAGGACUUCGACG |
| | | | CCAUCAUCGCCGAGCACUUCCAGCCCGGCGACUGCGUGCUGGAGAC |
| | | | CGACAUCGCCAGCUUCGACAAGAGCGAGGAUGACGCUAUGGCCCUG |
| | | | ACCGCUCUGAUGAUCCUGGAGGACCUGGGCGUGGACGCCGAGCUGC |
| | | | UCACCCUGAUCGAGGCUGCCUUCGGCGAGAUCAGCUCCAUCCACCU |
| | | | GCCCACCAAGACCAAGUUCAAGUUCGGCGCUAUGAUGAAAAGCGGA |
| | | | AUGUUCCUGACCCUGUUCGUGAACACCGUGAUCAACAUUGUGAUCG |
| | | | CCAGCAGGGUGCUGCGGGAGAGGCUGACCGGCAGCCCCUGCGCUGC |
| | | | CUUCAUCGGCGACGACAACAUCGUGAAGGGCGUGAAAAGCGACAAG |
| | | | CUGAUGGCCGACAGGUGCGCCACCUGGCUGAACAUGGAGGUGAAGA |
| | | | UCAUCGACGCCGUGGUGGGCGAGAAGGCCCCCUACUUCUGCGGCGG |
| | | | AUUCAUCCUGUGCGACAGCGUGACCGGCACCGCCUGCAGGGUGGCC |
| | | | GACCCCCUGAAGAGGCUGUUCAAGCUGGGCAAGCCACUGGCCGCUG |
| | | | ACGAUGAGCACGACGAUGACAGGCGGAGGGCCCUGCACGAGGAAAG |
| | | | CACCAGGUGGAACAGGGUGGGCAUCCUGAGCGAGCUGUGCAAGGCC |
| | | | GUGGAGAGCAGGUACGAGACCGUGGGCACCAGCAUCAUCGUGAUGG |
| | | | CUAUGACCACACUGGCCAGCUCCGUCAAGAGCUUCUCCUACCUGAG |
| | | | GGGGGCCCCUAUAACUCUCUACGGCUAACCUGAAUGGACUACGACA |
| | | | UAGUCUAGUCCGCCAAGGCCGCCACCAUGAGAGUGACAGCCCCUAG |
| | | | AACCUUACUGCUUCUGCUUUGGGGAGCUGUUGCUCUGACAGAGACA |
| | | | UGGGCUGGAUCUCUGAGCGAGGUGACCGGCCAGGGCCUGUGCAUCG |
| | | | GCGCCGUGCCCAAGACCCACCAGGUGCUGUGCAACACCACCCAGAA |
| | | | GACCAGCGACGGCAGCUACUACCUGGCCGCUCCCACCGGCACCACC |
| | | | UGGGCCUGCAGCACCGGCCUGACCCCUUGCAUCAGCACCACCAUCC |
| | | | UGAACCUGACCACCGACUACUGCGUGCUGGUGGAGCUGUGGCCCAG |
| | | | GGUGACCUACCACAGCCCCAGCUACGCCUACCACCAGUUCGAGAGG |
| | | | AGGGCCAAGUACAAGAGGGAGCCCGUGAGCCUGACCCUGGCCCUGC |
| | | | UGCUGGGCGGCCUGACAAUGGGCGGCAUCGCCGCCGGCGUGGGCAC |
| | | | CGGCACCACCGCCCUGGUGGCCACCCAGCAGUUCCAGCAGCUGCAG |
| | | | GCCGCCAUGCACGACGACCUGAAGGAGGUGGAGAAGUCCAUCACCA |
| | | | ACCUGGAGAAGUCCCUGACCAGCCUGAGCGAGGUGGUGCUGCAGAA |
| | | | CAGGAGGGGCCUGGACCUGCUGUUCCUGAAGGAGGGCGGCCUGUGC |
| | | | GCCGCCCUGAAGGAGGAGUGCUGCCUGUACGCCGACCACACCGGCC |
| | | | UGGUGAUCGUGGGCAUUGUCGCUGGCCUGGCCGUCCUCGCCGUGGU |
| | | | GGUGAUUGGAGCUGUGGUCGCAGCUGUUAUGUGCAGAAGAAAGUCA |
| | | | UCCGGCGGAAAGGGAGGCUCCUACUCUCAGGCUGCUUCUGCUACAG |
| | | | UGCCUAGAGCUCUUAUGUGUUUAUCUCAGCUGGGCGGCGGAGGCAG |
| | | | CGACUACAAGGACGACGAUGACAAGUAAACUCGAGUAUGUUACGUG |
| | | | CAAAGGUGAUUGUCACCCCCCGAAAGACCAUAUUGUGACACACCCU |
| | | | CAGUAUCACGCCCAAACAUUUACAGCCGCGGUGUCAAAAACCGCGU |
| | | | GGACGUGGUUAACAUCCCUGCUGGGAGGAUCAGCCGUAAUUAUUAU |
| | | | AAUUGGCUUGGUGCUGGCUACUAUUGUGGCCAUGUACGUGCUGACC |
| | | | AACCAGAAACAUAAUUGAAUACAGCAGCAAUUGGCAAGCUGCUUAC |
| | | | AUAGAACUCGCGGCGAUUGGCAUGCCGCCUUAAAAUUUUUAUUUUA |
| | | | UUUUUUCUUUUCUUUUCCGAAUCGGAUUUUGUUUUUAAUAUUUCAA |
| | | | AAAAAAAAAAAAAAAAAAAAAAAUCUAGAAAAAAAAAAAAAAAAAA |
| | | | AAAAAAAAAAAAAAAAAAAAAAAAAAAAAAAAAAAAAAAAAAAAAA |
| | | | AAAAAAAAAAAAAAAAAAAAAAAAAAAAAAAAAAAA |
| 3068 | STARR™ | 3068 | AUGGGCGGCGCAUGAGAGAAGCCCAGACCAAUUACCUACCCAAAAU |
| (SEQ | AH1A | | GGAGAAAGUUCACGUUGACAUCGAGGAAGACAGCCCAUUCCUCAGA |
| ID | 5- | | GCUUUGCAGCGGAGCUUCCCGCAGUUUGAGGUAGAAGCCAAGCAGG |
| NO: | FLAG | | UCACUGAUAAUGACCAUGCUAAUGCCAGAGCGUUUUCGCAUCUGGC |
| 108) | | | UUCAAAACUGAUCGAAACGGAGGUGGACCCAUCCGACACGAUCCUU |
| | | | GACAUUGGAAGUGCGCCCGCCCGCAGAAUGUAUUCUAAGCACAAGU |
| | | | AUCAUUGUAUCUGUCCGAUGAGAUGUGCGGAAGAUCCGGACAGAUU |
| | | | GUAUAAGUAUGCAACUAAGCUGAAGAAAAACUGUAAGGAAAUAACU |
| | | | GAUAAGGAAUUGGACAAGAAAAUGAAGGAGCUGGCCGCCGUCAUGA |
| | | | GCGACCCUGACCUGGAAACUGAGACUAUGUGCCUCCACGACGACGA |
| | | | GUCGUGUCGCUACGAAGGGCAAGUCGCUGUUUACCAGGAUGUAUAC |
| | | | GCCGUCGACGGCCCCACCAGCCUGUACCACCAGGCCAACAAGGGCG |
| | | | UGAGGGUGGCCUACUGGAUCGGCUUCGACACCACACCCUUCAUGUU |
| | | | CAAGAACCUGGCCGGCGCCUACCCCAGCUACAGCACCAACUGGGCC |
| | | | GACGAGACCGUGCUGACCGCCAGGAACAUCGGCCUGUGCAGCAGCG |
| | | | ACGUGAUGGAGAGGAGCCGGAGAGGCAUGAGCAUCCUGAGGAAGAA |
| | | | AUACCUGAAGCCCAGCAACAACGUGCUGUUCAGCGUGGGCAGCACC |
| | | | AUCUACCACGAGAAGAGGGACCUGCUCAGGAGCUGGCACCUGCCCA |
| | | | GCGUGUUCCACCUGAGGGGCAAGCAGAACUACACCUGCAGGUGCGA |
| | | | GACCAUCGUGAGCUGCGACGGCUACGUGGUGAAGAGGAUCGCCAUC |
| | | | AGCCCCGGCCUGUACGGCAAGCCCAGCGGCUACGCCGCUACAAUGC |
| | | | ACAGGGAGGGCUUCCUGUGCUGCAAGGUGACCGACACCCUGAACGG |
| | | | CGAGAGGGUGAGCUUCCCCGUGUGCACCUACGUGCCCGCCACCCUG |
| | | | UGCGACCAGAUGACCGGCAUCCUGGCCACCGACGUGAGCGCCGACG |
| | | | ACGCCCAGAAGCUGCUCGUGGGCCUGAACCAGAGGAUCGUGGUCAA |
| | | | CGGCAGGACCCAGAGGAACACCAACACAAUGAAGAACUACCUGCUG |
| | | | CCCGUGGUGGCCCAGGCUUUCGCCAGGUGGGCCAAGGAGUACAAGG |
| | | | AGGACCAGGAAGACGAGAGGCCCCUGGGCCUGAGGGACAGGCAGCU |
| | | | GGUGAUGGGCUGCUGCUGGGCCUUCAGGCGGCACAAGAUCACCAGC |
| | | | AUCUACAAGAGGCCCGACACCCAGACCAUCAUCAAGGUGAACAGCG |
| | | | ACUUCCACAGCUUCGUGCUGCCCAGGAUCGGCAGCAACACCCUGGA |
| | | | GAUCGGCCUGAGGACCCGGAUCAGGAAGAUGCUGGAGGAACACAAG |
| | | | GAGCCCAGCCCACUGAUCACCGCCGAGGACGUGCAGGAGGCCAAGU |
| | | | GCGCUGCCGACGAGGCCAAGGAGGUGAGGGAGGCCGAGGAACUGAG |
| | | | GGCCGCCCUGCCACCCCUGGCUGCCGACGUGGAGGAACCCACCCUG |
| | | | GAAGCCGACGUGGACCUGAUGCUGCAGGAGGCCGGCGCCGGAAGCG |
| | | | UGGAGACACCCAGGGGCCUGAUCAAGGUGACCAGCUACGACGGCGA |
| | | | GGACAAGAUCGGCAGCUACGCCGUGCUGAGCCCACAGGCCGUGCUG |
| | | | AAGUCCGAGAAGCUGAGCUGCAUCCACCCACUGGCCGAGCAGGUGA |
| | | | UCGUGAUCACCCACAGCGGCAGGAAGGGCAGGUACGCCGUGGAGCC |
| | | | CUACCACGGCAAGGUGGUCGUGCCCGAGGGCCACGCCAUCCCCGUG |
| | | | CAGGACUUCCAGGCCCUGAGCGAGAGCGCCACCAUCGUGUACAACG |
| | | | AGAGGGAGUUCGUGAACAGGUACCUGCACCAUAUCGCCACCCACGG |
| | | | CGGAGCCCUGAACACCGACGAGGAAUACUACAAGACCGUGAAGCCC |
| | | | AGCGAGCACGACGGCGAGUACCUGUACGACAUCGACAGGAAGCAGU |
| | | | GCGUGAAGAAAGAGCUGGUGACCGGCCUGGGACUGACCGGCGAGCU |
| | | | GGUGGACCCACCCUUCCACGAGUUCGCCUACGAGAGCCUGAGGACC |
| | | | AGACCCGCCGCUCCCUACCAGGUGCCCACCAUCGGCGUGUACGGCG |
| | | | UGCCCGGCAGCGGAAAGAGCGGCAUCAUCAAGAGCGCCGUGACCAA |
| | | | GAAAGACCUGGUGGUCAGCGCCAAGAAAGAGAACUGCGCCGAGAUC |
| | | | AUCAGGGACGUGAAGAAGAUGAAAGGCCUGGACGUGAACGCGCGCA |
| | | | CCGUGGACAGCGUGCUGCUGAACGGCUGCAAGCACCCCGUGGAGAC |
| | | | CCUGUACAUCGACGAGGCCUUCGCUUGCCACGCCGGCACCCUGAGG |
| | | | GCCCUGAUCGCCAUCAUCAGGCCCAAGAAAGCCGUGCUGUGCGGCG |
| | | | ACCCCAAGCAGUGCGGCUUCUUCAACAUGAUGUGCCUGAAGGUGCA |
| | | | CUUCAACCACGAGAUCUGCACCCAGGUGUUCCACAAGAGCAUCAGC |
| | | | AGGCGGUGCACCAAGAGCGUGACCAGCGUCGUGAGCACCCUGUUCU |
| | | | ACGACAAGAAAAUGAGGACCACCAACCCCAAGGAGACCAAAAUCGU |
| | | | GAUCGACACCACAGGCAGCACCAAGCCCAAGCAGGACGACCUGAUC |
| | | | CUGACCUGCUUCAGGGGCUGGGUGAAGCAGCUGCAGAUCGACUACA |
| | | | AGGGCAACGAGAUCAUGACCGCCGCUGCCAGCCAGGGCCUGACCAG |
| | | | GAAGGGCGUGUACGCCGUGAGGUACAAGGUGAACGAGAACCCACUG |
| | | | UACGCUCCCACCAGCGAGCACGUGAACGUGCUGCUGACCAGGACCG |
| | | | AGGACAGGAUCGUGUGGAAGACCCUGGCCGGCGACCCCUGGAUCAA |
| | | | GACCCUGACCGCCAAGUACCCCGGCAACUUCACCGCCACCAUCGAA |
| | | | GAGUGGCAGGCCGAGCACGACGCCAUCAUGAGGCACAUCCUGGAGA |
| | | | GGCCCGACCCCACCGACGUGUUCCAGAACAAGGCCAACGUGUGCUG |
| | | | GGCCAAGGCCCUGGUGCCCGUGCUGAAGACCGCCGGCAUCGACAUG |
| | | | ACCACAGAGCAGUGGAACACCGUGGACUACUUCGAGACCGACAAGG |
| | | | CCCACAGCGCCGAGAUCGUGCUGAACCAGCUGUGCGUGAGGUUCUU |
| | | | CGGCCUGGACCUGGACAGCGGCCUGUUCAGCGCCCCCACCGUGCCA |
| | | | CUGAGCAUCAGGAACAACCACUGGGACAACAGCCCCAGCCCAAACA |
| | | | UGUACGGCCUGAACAAGGAGGUGGUCAGGCAGCUGAGCAGGCGGUA |
| | | | CCCACAGCUGCCCAGGGCCGUGGCCACCGGCAGGGUGUACGACAUG |
| | | | AACACCGGCACCCUGAGGAACUACGACCCCAGGAUCAACCUGGUGC |
| | | | CCGUGAACAGGCGGCUGCCCCACGCCCUGGUGCUGCACCACAACGA |
| | | | GCACCCACAGAGCGACUUCAGCUCCUUCGUGAGCAAGCUGAAAGGC |
| | | | AGGACCGUGCUGGUCGUGGGCGAGAAGCUGAGCGUGCCCGGCAAGA |
| | | | UGGUGGACUGGCUGAGCGACAGGCCCGAGGCCACCUUCCGGGCCAG |
| | | | GCUGGACCUCGGCAUCCCCGGCGACGUGCCCAAGUACGACAUCAUC |
| | | | UUCGUGAACGUCAGGACCCCAUACAAGUACCACCAUUACCAGCAGU |
| | | | GCGAGGACCACGCCAUCAAGCUGAGCAUGCUGACCAAGAAGGCCUG |
| | | | CCUGCACCUGAACCCCGGAGGCACCUGCGUGAGCAUCGGCUACGGC |
| | | | UACGCCGACAGGGCCAGCGAGAGCAUCAUUGGCGCCAUCGCCAGGC |
| | | | UGUUCAAGUUCAGCAGGGUGUGCAAACCCAAGAGCAGCCUGGAGGA |
| | | | AACCGAGGUGCUGUUCGUGUUCAUCGGCUACGACCGGAAGGCCAGG |
| | | | ACCCACAACCCCUACAAGCUGAGCAGCACCCUGACAAACAUCUACA |
| | | | CCGGCAGCAGGCUGCACGAGGCCGGCUGCGCCCCCAGCUACCACGU |
| | | | GGUCAGGGGCGAUAUCGCCACCGCCACCGAGGGCGUGAUCAUCAAC |
| | | | GCUGCCAACAGCAAGGGCCAGCCCGGAGGCGGAGUGUGCGGCGCCC |
| | | | UGUACAAGAAGUUCCCCGAGAGCUUCGACCUGCAGCCCAUCGAGGU |
| | | | GGGCAAGGCCAGGCUGGUGAAGGGCGCCGCUAAGCACAUCAUCCAC |
| | | | GCCGUGGGCCCCAACUUCAACAAGGUGAGCGAGGUGGAAGGCGACA |
| | | | AGCAGCUGGCCGAAGCCUACGAGAGCAUCGCCAAGAUCGUGAACGA |
| | | | CAAUAACUACAAGAGCGUGGCCAUCCCACUGCUCAGCACCGGCAUC |
| | | | UUCAGCGGCAACAAGGACAGGCUGACCCAGAGCCUGAACCACCUGC |
| | | | UCACCGCCCUGGACACCACCGAUGCCGACGUGGCCAUCUACUGCAG |
| | | | GGACAAGAAGUGGGAGAUGACCCUGAAGGAGGCCGUGGCCAGGCGG |
| | | | GAGGCCGUGGAAGAGAUCUGCAUCAGCGACGACUCCAGCGUGACCG |
| | | | AGCCCGACGCCGAGCUGGUGAGGGUGCACCCCAAGAGCUCCCUGGC |
| | | | CGGCAGGAAGGGCUACAGCACCAGCGACGGCAAGACCUUCAGCUAC |
| | | | CUGGAGGGCACCAAGUUCCACCAGGCCGCUAAGGACAUCGCCGAGA |
| | | | UCAACGCUAUGUGGCCCGUGGCCACCGAGGCCAACGAGCAGGUGUG |
| | | | CAUGUACAUCCUGGGCGAGAGCAUGUCCAGCAUCAGGAGCAAGUGC |
| | | | CCCGUGGAGGAAAGCGAGGCCAGCACACCACCCAGCACCCUGCCCU |
| | | | GCCUGUGCAUCCACGCUAUGACACCCGAGAGGGUGCAGCGGCUGAA |
| | | | GGCCAGCAGGCCCGAGCAGAUCACCGUGUGCAGCUCCUUCCCACUG |
| | | | CCCAAGUACAGGAUCACCGGCGUGCAGAAGAUCCAGUGCAGCCAGC |
| | | | CCAUCCUGUUCAGCCCAAAGGUGCCCGCCUACAUCCACCCCAGGAA |
| | | | GUACCUGGUGGAGACCCCACCCGUGGACGAGACACCCGAGCCAAGC |
| | | | GCCGAGAACCAGAGCACCGAGGGCACACCCGAGCAGCCACCCCUGA |
| | | | UCACCGAGGACGAGACAAGGACCCGGACCCCAGAGCCCAUCAUUAU |
| | | | CGAGGAAGAGGAAGAGGACAGCAUCAGCCUGCUGAGCGACGGCCCC |
| | | | ACCCACCAGGUGCUGCAGGUGGAGGCCGACAUCCACGGCCCACCCA |
| | | | GCGUGUCCAGCUCCAGCUGGAGCAUCCCACACGCCAGCGACUUCGA |
| | | | CGUGGACAGCCUGAGCAUCCUGGACACCCUGGAGGGCGCCAGCGUG |
| | | | ACCUCCGGCGCCACCAGCGCCGAGACCAACAGCUACUUCGCCAAGA |
| | | | GCAUGGAGUUCCUGGCCAGGCCCGUGCCAGCUCCCAGGACCGUGUU |
| | | | CAGGAACCCACCCCACCCAGCUCCCAGGACCAGGACCCCAAGCCUG |
| | | | GCUCCCAGCAGGGCCUGCAGCAGGACCAGCCUGGUGAGCACCCCAC |
| | | | CCGGCGUGAACAGGGUGAUCACCAGGGAGGAACUGGAGGCCCUGAC |
| | | | ACCCAGCAGGACCCCCAGCAGGUCCGUGAGCAGGACUAGUCUGGUG |
| | | | UCCAACCCACCCGGCGUGAACAGGGUGAUCACCAGGGAGGAAUUCG |
| | | | AGGCCUUCGUGGCCCAGCAACAGAGACGGUUCGACGCCGGCGCCUA |
| | | | CAUCUUCAGCAGCGACACCGGCCAGGGACACCUGCAGCAAAAGAGC |
| | | | GUGAGGCAGACCGUGCUGAGCGAGGUGGUGCUGGAGAGGACCGAGC |
| | | | UGGAAAUCAGCUACGCCCCCAGGCUGGACCAGGAGAAGGAGGAACU |
| | | | GCUCAGGAAGAAACUGCAGCUGAACCCCACCCCAGCCAACAGGAGC |
| | | | AGGUACCAGAGCAGGAAGGUGGAGAACAUGAAGGCCAUCACCGCCA |
| | | | GGCGGAUCCUGCAGGGCCUGGGACACUACCUGAAGGCCGAGGGCAA |
| | | | GGUGGAGUGCUACAGGACCCUGCACCCCGUGCCACUGUACAGCUCC |
| | | | AGCGUGAACAGGGCCUUCUCCAGCCCCAAGGUGGCCGUGGAGGCCU |
| | | | GCAACGCUAUGCUGAAGGAGAACUUCCCCACCGUGGCCAGCUACUG |
| | | | CAUCAUCCCCGAGUACGACGCCUACCUGGACAUGGUGGACGGCGCC |
| | | | AGCUGCUGCCUGGACACCGCCAGCUUCUGCCCCGCCAAGCUGAGGA |
| | | | GCUUCCCCAAGAAACACAGCUACCUGGAGCCCACCAUCAGGAGCGC |
| | | | CGUGCCCAGCGCCAUCCAGAACACCCUGCAGAACGUGCUGGCCGCU |
| | | | GCCACCAAGAGGAACUGCAACGUGACCCAGAUGAGGGAGCUGCCCG |
| | | | UGCUGGACAGCGCUGCCUUCAACGUGGAGUGCUUCAAGAAAUACGC |
| | | | CUGCAACAACGAGUACUGGGAGACCUUCAAGGAGAACCCCAUCAGG |
| | | | CUGACCGAAGAGAACGUGGUGAACUACAUCACCAAGCUGAAGGGCC |
| | | | CCAAGGCCGCUGCCCUGUUCGCUAAGACCCACAACCUGAACAUGCU |
| | | | GCAGGACAUCCCAAUGGACAGGUUCGUGAUGGACCUGAAGAGGGAC |
| | | | GUGAAGGUGACACCCGGCACCAAGCACACCGAGGAGAGGCCCAAGG |
| | | | UGCAGGUGAUCCAGGCCGCUGACCCACUGGCCACCGCCUACCUGUG |
| | | | CGGCAUCCACAGGGAGCUGGUGAGGCGGCUGAACGCCGUGCUGCUG |
| | | | CCCAACAUCCACACCCUGUUCGACAUGAGCGCCGAGGACUUCGACG |
| | | | CCAUCAUCGCCGAGCACUUCCAGCCCGGCGACUGCGUGCUGGAGAC |
| | | | CGACAUCGCCAGCUUCGACAAGAGCGAGGAUGACGCUAUGGCCCUG |
| | | | ACCGCUCUGAUGAUCCUGGAGGACCUGGGCGUGGACGCCGAGCUGC |
| | | | UCACCCUGAUCGAGGCUGCCUUCGGCGAGAUCAGCUCCAUCCACCU |
| | | | GCCCACCAAGACCAAGUUCAAGUUCGGCGCUAUGAUGAAAAGCGGA |
| | | | AUGUUCCUGACCCUGUUCGUGAACACCGUGAUCAACAUUGUGAUCG |
| | | | CCAGCAGGGUGCUGCGGGAGAGGCUGACCGGCAGCCCCUGCGCUGC |
| | | | CUUCAUCGGCGACGACAACAUCGUGAAGGGCGUGAAAAGCGACAAG |
| | | | CUGAUGGCCGACAGGUGCGCCACCUGGCUGAACAUGGAGGUGAAGA |
| | | | UCAUCGACGCCGUGGUGGGCGAGAAGGCCCCCUACUUCUGCGGCGG |
| | | | AUUCAUCCUGUGCGACAGCGUGACCGGCACCGCCUGCAGGGUGGCC |
| | | | GACCCCCUGAAGAGGCUGUUCAAGCUGGGCAAGCCACUGGCCGCUG |
| | | | ACGAUGAGCACGACGAUGACAGGCGGAGGGCCCUGCACGAGGAAAG |
| | | | CACCAGGUGGAACAGGGUGGGCAUCCUGAGCGAGCUGUGCAAGGCC |
| | | | GUGGAGAGCAGGUACGAGACCGUGGGCACCAGCAUCAUCGUGAUGG |
| | | | CUAUGACCACACUGGCCAGCUCCGUCAAGAGCUUCUCCUACCUGAG |
| | | | GGGGGCCCCUAUAACUCUCUACGGCUAACCUGAAUGGACUACGACA |
| | | | UAGUCUAGUCCGCCAAGGCCGCCACCAUGAGAGUGACAGCCCCUAG |
| | | | AACCUUACUGCUUCUGCUUUGGGGAGCUGUUGCUCUGACAGAGACA |
| | | | UGGGCUGGAUCUUACCACAGCCCCAGCUACGCCUACCACCAGUUCG |
| | | | AGAGGGGGGGAGGAGGCUCCGGGGGAGGAGGCUCCCUGAAGAUCAG |
| | | | CCAGGCCGUGCACGCCGCCCACGCCGAGAUCAACGAGGCCGGCCGG |
| | | | GAGGUGAUCGUGGGCAUUGUCGCUGGCCUGGCCGUCCUCGCCGUGG |
| | | | UGGUGAUUGGAGCUGUGGUCGCAGCUGUUAUGUGCAGAAGAAAGUC |
| | | | AUCCGGCGGAAAGGGAGGCUCCUACUCUCAGGCUGCUUCUGCUACA |
| | | | GUGCCUAGAGCUCUUAUGUGUUUAUCUCAGCUGGGCGGCGGAGGCA |
| | | | GCGACUACAAGGACGACGAUGACAAGUAAACUCGAGUAUGUUACGU |
| | | | GCAAAGGUGAUUGUCACCCCCCGAAAGACCAUAUUGUGACACACCC |
| | | | UCAGUAUCACGCCCAAACAUUUACAGCCGCGGUGUCAAAAACCGCG |
| | | | UGGACGUGGUUAACAUCCCUGCUGGGAGGAUCAGCCGUAAUUAUUA |
| | | | UAAUUGGCUUGGUGCUGGCUACUAUUGUGGCCAUGUACGUGCUGAC |
| | | | CAACCAGAAACAUAAUUGAAUACAGCAGCAAUUGGCAAGCUGCUUA |
| | | | CAUAGAACUCGCGGCGAUUGGCAUGCCGCCUUAAAAUUUUUAUUUU |
| | | | AUUUUUUCUUUUCUUUUCCGAAUCGGAUUUUGUUUUUAAUAUUUCA |
| | | | AAAAAAAAAAAAAAAAAAAAAAAAUCUAGAAAAAAAAAAAAAAAAA |
| | | | AAAAAAAAAAAAAAAAAAAAAAAAAAAAAAAAAAAAAAAAAAAAAA |
| | | | AAAAAAAAAAAAAAAAAAAAAAAAAAAAAAAAAAAAA |

non structural protein of SINV

| mARM | | |
|---|---|---|
| # | | |
| 2842 | SINV | MEKPVVNVDVDPQSPFVVQLQKSFPQFEVVAQQVTPNDHANARAFS |
| and | nsP1- | HLASKLIELEVPTTATILDIGSAPARRMFSEHQYHCVCPMRSPEDP |
| 2862 | 4 AA | DRMMKYASKLAEKACKITNKNLHEKIKDLRTVLDTPDAETPSLCFH |
| (SEQ | | NDVTCNMRAEYSVMQDVYINAPGTIYHQAMKGVRTLYWIGFDTTQF |
| ID | | MFSAMAGSYPAYNTNWADEKVLEARNIGLCSTKLSEGRTGKLSIMR |
| NO: | | KKELKPGSRVYFSVGSTLYPEHRASLQSWHLPSVFHLNGKQSYTCR |
| 109) | | CDTVVSCEGYVVKKITISPGITGETVGYAVTHNSEGFLLCKVTDTV |
| | | KGERVSFPVCTYIPATICDQMTGIMATDISPDDAQKLLVGLNQRIV |
| | | INGRTNRNTNTMQNYLLPIIAQGFSKWAKERKDDLDNEKMLGTRER |
| | | KLTYGCLWAFRTKKVHSFYRPPGTQTCVKVPASFSAFPMSSVWTTS |
| | | LPMSLRQKLKLALQPKKEEKLLQVSEELVMEAKAAFEDAQEEARAE |
| | | KLREALPPLVADKGIEAAAEVVCEVEGLQADIGAALVETPRGHVRI |
| | | IPQANDRMIGQYIVVSPNSVLKNAKLAPAHPLADQVKIITHSGRSG |
| | | RYAVEPYDAKVLMPAGGAVPWPEFLALSESATLVYNEREFVNRKLY |
| | | HIAMHGPAKNTEEEQYKVTKAELAETEYVFDVDKKRCVKKEEASGL |
| | | VLSGELTNPPYHELALEGLKTRPAVPYKVETIGVIGTPGSGKSAII |
| | | KSTVTARDLVTSGKKENCREIEADVLRLRGMQITSKTVDSVMLNGC |
| | | HKAVEVLYVDEAFACHAGALLALIAIVRPRKKVVLCGDPMQCGFFN |
| | | MMQLKVHFNHPEKDICTKTFYKYISRRCTQPVTAIVSTLHYDGKMK |
| | | TTNPCKKNIEIDITGATKPKPGDIILTCFRGWVKQLQIDYPGHEVM |
| | | TAAASQGLTRKGVYAVRQKVNENPLYAITSEHVNVLLTRTEDRLVW |
| | | KTLQGDPWIKQLTNIPKGNFQATIEDWEAEHKGIIAAINSPTPRAN |
| | | PFSCKTNVCWAKALEPILATAGIVLTGCQWSELFPQFADDKPHSAI |
| | | YALDVICIKFFGMDLTSGLFSKQSIPLTYHPADSARPVAHWDNSPG |
| | | TRKYGYDHAIAAELSRRFPVFQLAGKGTQLDLQTGRTRVISAQHNL |
| | | VPVNRNLPHALVPEYKEKQPGPVEKFLNQFKHHSVLVVSEEKIEAP |
| | | RKRIEWIAPIGIAGADKNYNLAFGFPPQARYDLVFINIGTKYRNHH |
| | | FQQCEDHAATLKTLSRSALNCLNPGGTLVVKSYGYADRNSEDVVTA |
| | | LARKFVRVSAARPDCVSSNTEMYLIFRQLDNSRTRQFTPHHLNCVI |
| | | SSVYEGTRDGVGAAPSYRTKRENIADCQEEAVVNAANPLGRPGEGV |
| | | CRAIYKRWPTSFTDSATETGTARMTVCLGKKVIHAVGPDFRKHPEA |
| | | EALKLLQNAYHAVADLVNEHNIKSVAIPLLSTGIYAAGKDRLEVSL |
| | | NCLTTALDRTDADVTIYCLDKKWKERIDAALQLKESVTELKDEDME |
| | | IDDELVWIHPDSCLKGRKGFSTTKGKLYSYFEGTKFHQAAKDMAEI |
| | | KVLFPNDQESNEQLCAYILGETMEAIREKCPVDHNPSSSPPKTLPC |
| | | LCMYAMTPERVHRLRSNNVKEVTVCSSTPLPKHKIKNVQKVQCTKV |
| | | VLFNPHTPAFVPARKYIEVPEQPTAPPAQAEEAPEVVATPSPSTAD |
| | | NTSLDVTDISLDMDDSSEGSLFSSFSGSDNSITSMDSWSSGPSSLE |
| | | IVDRRQVVVADVHAVQEPAPIPPPRLKKMARLAAARKEPTPPASNS |
| | | SESLHLSFGGVSMSLGSIFDGETARQAAVQPLATGPTDVPMSFGSF |
| | | SDGEIDELSRRVTESEPVLFGSFEPGEVNSIISSRSAVSFPLRKQR |
| | | RRRRSRRTEY*LTGVGGYIFSTDTGPGHLQKKSVLQNQLTEPTLER |
| | | NVLERIHAPVLDTSKEEQLKLRYQMMPTEANKSRYQSRKVENQKAI |
| | | TTERLLSGLRLYNSATDQPECYKITYPKPLYSSSVPANYSDPQFAV |
| | | AVCNNYLHENYPTVASYQITDEYDAYLDMVDGTVACLDTATFCPAK |
| | | LRSYPKKHEYRAPNIRSAVPSAMQNTLQNVLIAATKRNCNVTQMRE |
| | | LPTLDSATFNVECFRKYACNDEYWEEFARKPIRITTEFVTAYVARL |
| | | KGPKAAALFAKTYNLVPLQEVPMDRFVMDMKRDVKVTPGTKHTEER |
| | | PKVQVIQAAEPLATAYICGIHRELVRRLTAVLLPNIHTLFDMSAED |
| | | FDAIIAEHFKQGDPVLETDIASFDKSQDDAMALTGLMILEDLGVDQ |
| | | PLLDLIECAFGEISSTHLPTGTRFKFGAMMKSGMFLTLFVNTVLNV |
| | | VIASRVLEERLKTSRCAAFIGDDNIIHGVVSDKEMAERCATWLNME |
| | | VKIIDAVIGERPPYFCGGFILQDSVTSTACRVADPLKRLFKLGKPL |
| | | PADDEQDEDRRRALLDETKAWFRVGITGTLAVAVTTRYEVDNITPV |
| | | LLALRTFAQSKRAFQAIRGEIKHLYGGPK |

### Example 11

This example describes analysis of the immunogenicity of influenza hemagglutinin (HA) expressed from self-replicating RNA or mRNA.

Self-replicating RNA and mRNA vaccine constructs were designed to encode the full-length hemagglutinin (HA) protein from influenza virus A/California/07/2009 (H1N1) (SEQ ID NO:113 and 114). As described above for Example 1, the mRNA vaccine construct encoding HA included a tobacco etch virus (TEV) 5′ UTR and a Xenopus beta-globin (Xbg) 3′ UTR. Both self-replicating RNA (SEQ ID NO:56; entire RNA mARM3039) and mRNA vaccine constructs (SEQ ID NO:116; entire RNA sequence mARM3038) were encapsulated in the same lipid nanoparticle (LNP) composition that included four lipid excipients (an ionizable cationic lipid, 1,2-distearoyl-sn-glycero-3-phosphocholine (DSPC), cholesterol, and PEG2000-DMG) dispersed in HEPES buffer (pH 8.0) containing sodium chloride and the cryoprotectants sucrose and glycerol. The N:P ratio of complexing lipid and RNA was approximately 9:1. The ionizable cationic lipid had the following structure:

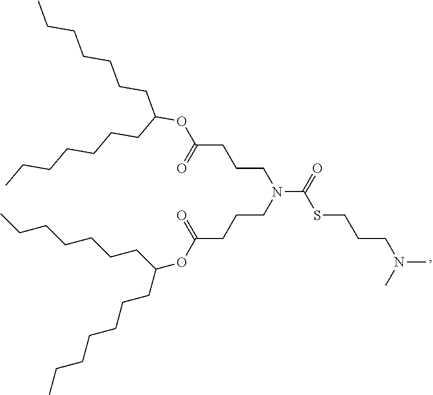 [SMILES: CCCCCCCC(CCCCCCC)OC(=O)CCCN(CCCC(=O)OC(CCCCCCC)CCCCCCC)C(=O)SCCCN(C)C]

Five female, 8-10 week old Balb/c mice were injected intramuscularly with 2 mg of mRNA or self-replicating RNA encoding HA. Mice were bled on days 14, 28, 42, and 56, followed by hemagglutination inhibition (HAI) assay using serially diluted sera. The reciprocal of the highest dilution of serum that caused inhibition of hemagglutination was considered the HAI titer, with a titer of 1/40 being protective against influenza virus infection and four-fold higher titers than baseline indicating seroconversion.

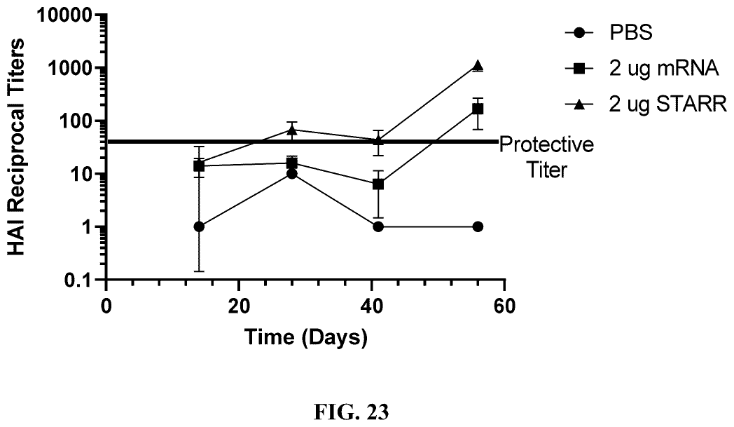

Results in FIG. 23 show that greater HAI titers were obtained with self-replicating RNA encoding HA as compared to mRNA encoding HA. HAI titers for the self-replicating RNA construct encoding HA were greater than HAI titers for the mRNA encoding HA at all time points beginning at day 28. In addition, protective HAI titers were seen for the self-replicating RNA construct encoding HA beginning at day 28 that were maintained for at least 56 days. By contrast, mRNA encoding HA showed protective HAI titers only at day 56 that were lower than HAI titers seen for the self-replicating RNA HA construct. At all other time points, HAI titers for the mRNA construct encoding HA were below the protective titer threshold, with an HAI titer that was comparable to injection with PBS control at day 28.

These results show that the self-replicating RNA construct encoding HA elicited protective HA antibody titers, with greater HAI titers as compared to the mRNA construct encoding HA.

### Example 12

This example describes dsRNA production and luciferase expression for self-replicating RNA.

Several self-replicating RNA systems from different alphaviruses were tested for expression in vitro using either green fluorescent protein (GFP) or firefly luciferase (Luc) as reporter genes. Initial transfection of cells with increasing amounts of self-replicating RNA resulted in expression of reporter genes at a lower dose compared to mRNA. However, as the amount of input self-replicating RNA increased, detectable expression of the reporter gene decreased.

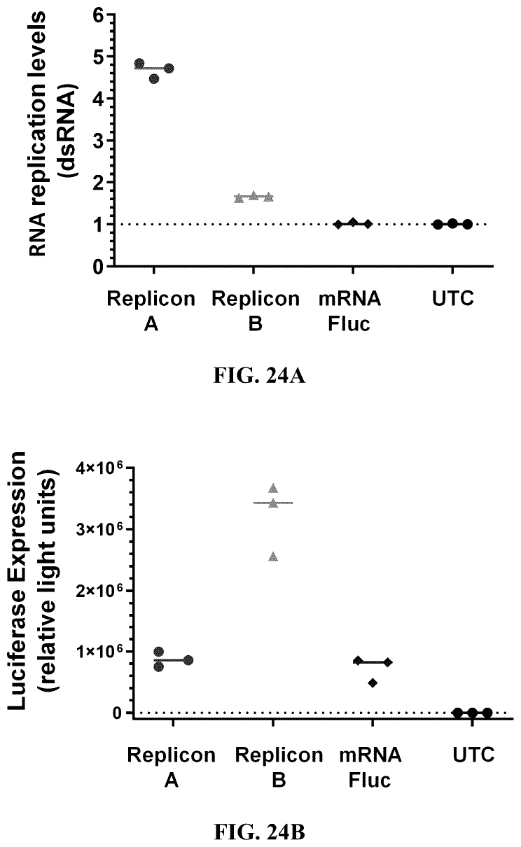

Self-replicating RNA produces double stranded RNA (dsRNA) as an intermediate in the amplification process. Overproduction of dsRNA can suppress translation. To evaluate the effect of dsRNA production on transgene expression, dsRNA and the expression of reporter gene luciferase were measured simultaneously. HEK293 cells were transfected with 2 μg of replicon A (SEQ ID NO:115; entire RNA sequence mARM2826) or replicon B (SEQ ID NO:100, entire RNA sequence mARM2809) self-replicating RNA, or mRNA expressing Luc (SEQ ID NO:102, entire mRNA sequence mARM1782) using a commercial RNA transfection reagent. Untransfected cells (UTC) served as a control. dsRNA production (FIG. 24A) was quantified using immunohistochemical staining for dsRNA, followed by fluorescence quantification using a fluorescence scanner 24 hours after transfection. Luciferase expression (FIG. 24B) was assayed by measuring bioluminescence in parallel.

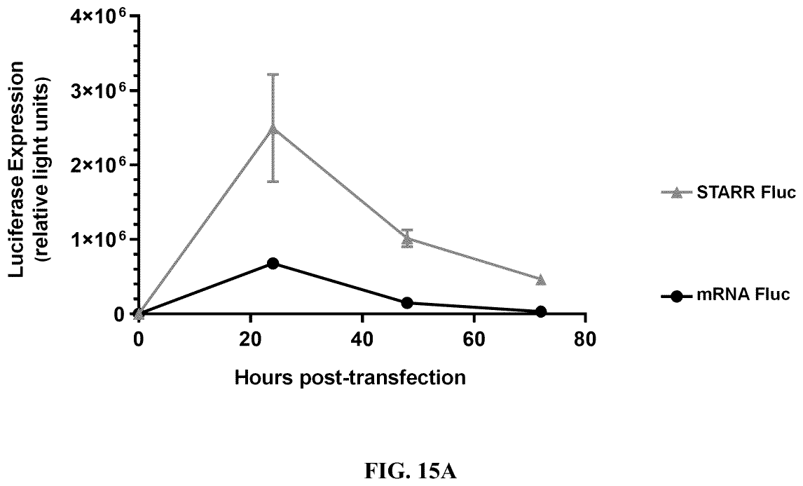

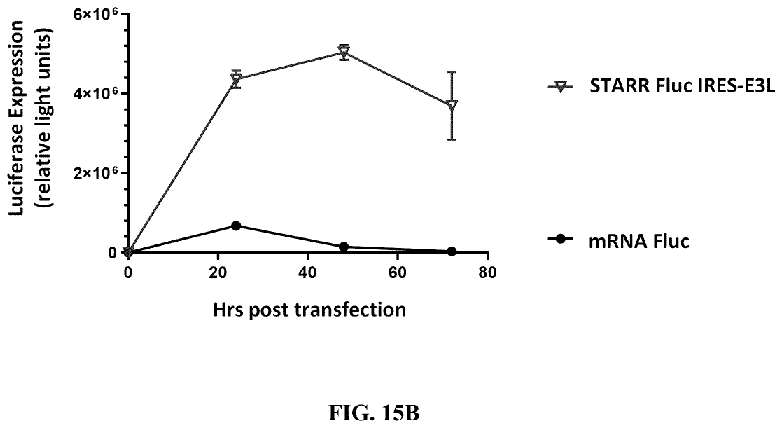

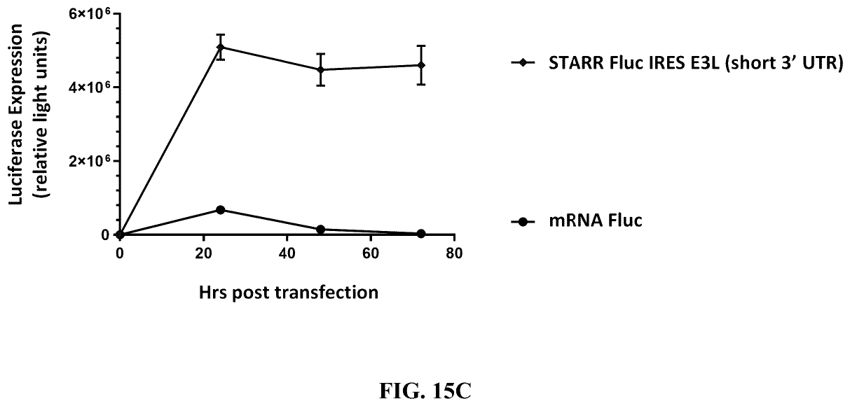

Replicon A produced a 3-fold higher level of dsRNA than replicon B 24 hrs after transfection (FIG. 24A). However, replicon B produced a 2.4-fold higher expression level of luciferase compared to replicon A. Furthermore, the level of luciferase expression from replicon A was equivalent to that observed for mRNA. Thus, even though replicon A had the ability to amplify the amount of replicon RNA and transcribed mRNA encoding luciferase, translation of the amplified mRNA was inhibited, consistent with overproduction of dsRNA inhibiting translation. Furthermore, higher levels of luciferase gene expression were seen for replicon RNA as compared to mRNA at 24, 48, and 72 hours after transfection of HEK293 cells (FIG. 15A). Self-replicating RNA with an expression cassette that included a luciferase reporter gene followed by an IRES and E3L also showed robust luciferase expression (FIGS. 15B, 15C; SEQ ID NOs: 128 and 129). Luciferase expression was also seen for a self-replicating RNA that expressed E3L from a first subgenomic promoter and a luciferase reporter gene from a second subgenomic promoter located 3′ of the E3L open reading frame (not shown). Thus, not only did replicon RNA produce higher levels of luciferase gene expression compared to mRNA, but replicon RNA also showed increased duration of expression over a 72-hr period.

### Example 13

This example describes immunogenicity of liquid and lyophilized self-replicating RNA formulations. Immunogenicity of self-replicating RNA (SEQ ID NO:125) formulated as a lyophilized lipid nanoparticle (LYO-LNP) was tested in BALB/c mice in two separate preclinical studies and compared with the liquid (frozen) LNP formulation (Liquid-LNP). Each study included the use of a PBS dosing group as a negative control and a Liquid dosing group (Liquid-LNP) as a positive control. Both LYO-LNP and Liquid-LNP formulations were dosed at 0.2 and 2 μg. There were n=5 animals per dose group in each study. Test formulations were administered intramuscularly (IM) and serum was collected at various timepoints (Days 10, 19, 31 for the first study and Days 10, 20, 30 for the second study) post-immunization to measure the production of anti-SARS-CoV-2 spike protein IgG using a Luminex bead fluorescent assay.

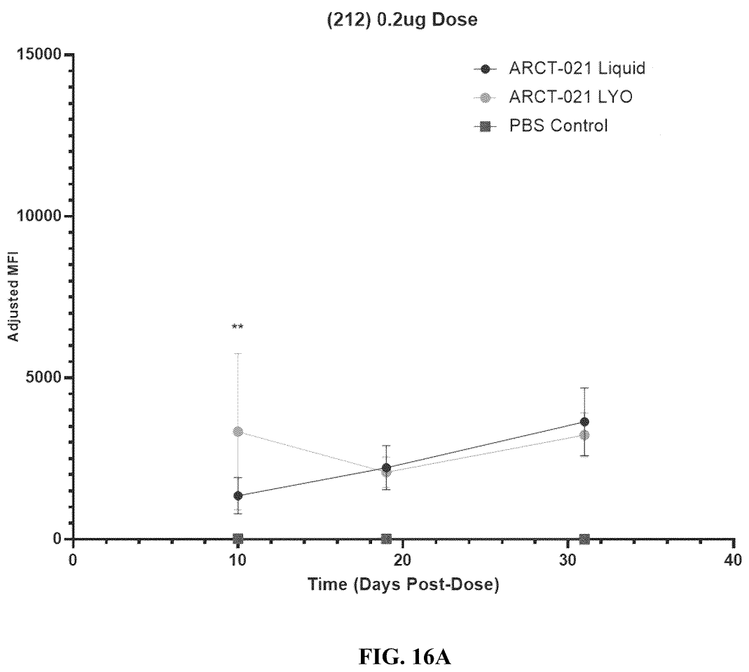

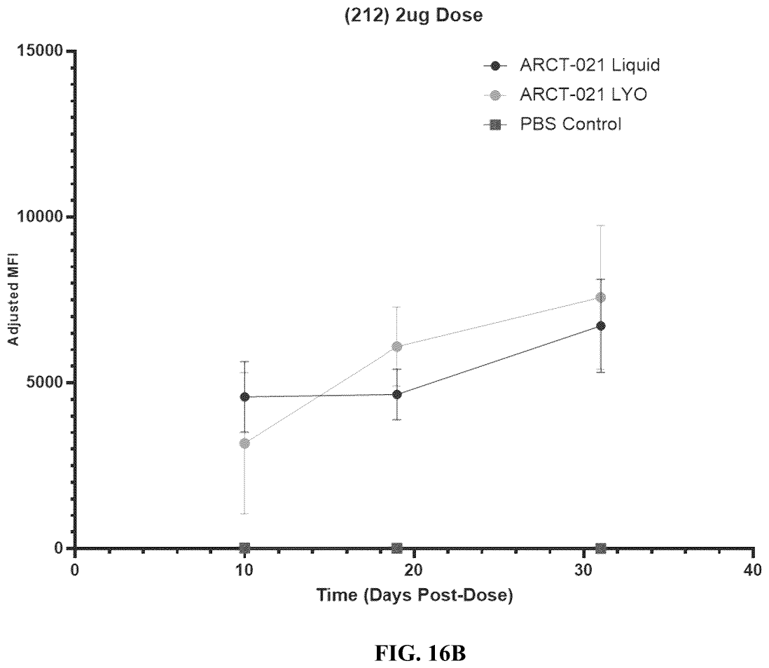

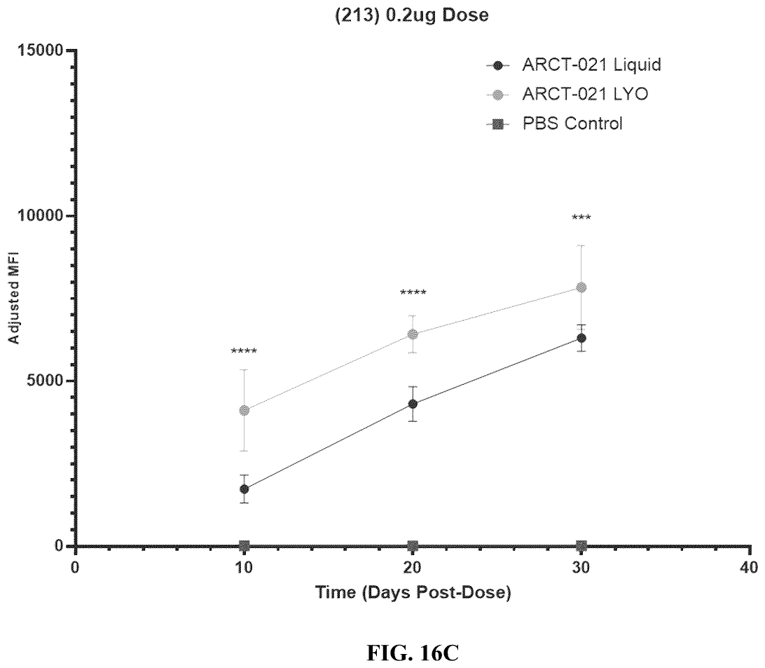

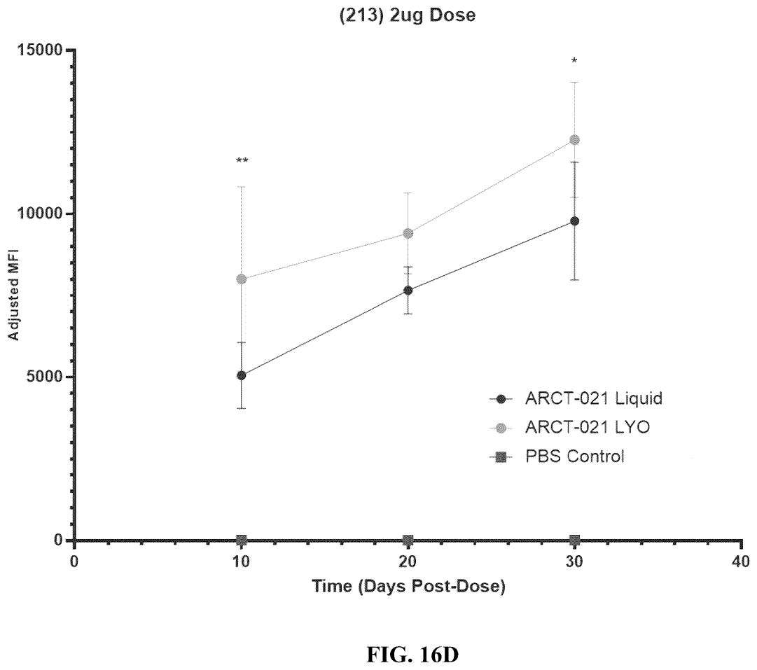

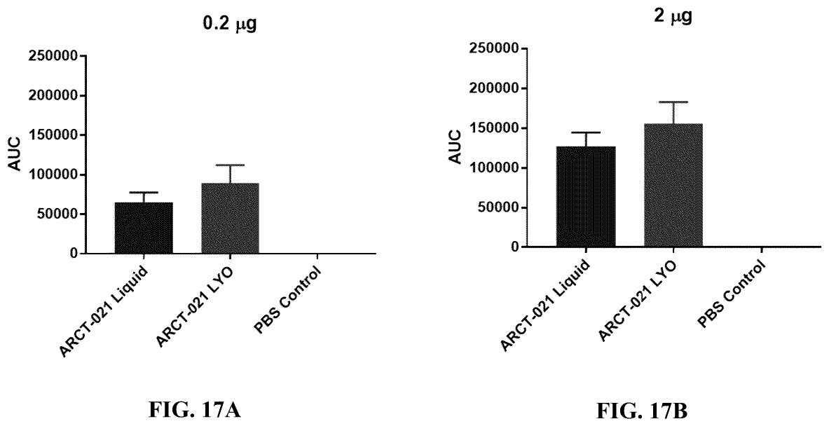

In both studies, anti-SARS-CoV-2 spike protein IgGs were detected in serum in a time- and dose-dependent manner for both Liquid-LNP and LYO-LNP formulations, whereas PBS injection did not elicit an immunogenic response (FIG. 16A-16D). There was no statistical difference in immunogenicity seen between Liquid-LNP and LYO-LNP dose groups in the first study, whereas LYO-LNP produced statistically different and greater IgG than Liquid-LNP in the second study. Without being limited by theory, under-powering (n=5/group) of these two separate studies may have contributed to the statistical differences in immunogenicity results observed in the two studies. In combining the results of both studies, no statistically significant differences were observed between Liquid-LNP and LYO-LNP formulations at the 0.2 and 2 μg dose levels (FIG. 17A, 17B). Taken together, the results of these studies demonstrate that the immunogenicity of the liquid and lyophilized formulations were comparable.

In summary, the liquid and lyophilized formulations of the self-replicating RNA vaccine (SEQ ID NO:125) showed comparable immunogenicity. The vaccine can induce effective, adaptive humoral (neutralizing antibodies) and cellular (CD8+) immune responses targeting the SARS-CoV-2 S glycoprotein. The vaccine also elicits induction of anti-spike glycoprotein antibodies (IgG) levels that are higher than a conventional mRNA vaccine and also induces production of IgG antibodies at a faster rate than a conventional mRNA vaccine. It continues to produce increasing levels of IgG up to 50 days post vaccination whereas the conventional mRNA vaccine plateaus by day 10 post vaccination. It produces an RNA dose-dependent increase in CD8+T lymphocytes and a balanced, Th1 dominant CD4+T helper cell immune response with no skew towards a Th2 response.

Any and all references and citations to other documents, such as patents, patent applications, patent publications, journals, books, papers, web contents, that have been made throughout this disclosure are hereby incorporated herein in their entirety for all purposes.

Although the invention has been described with reference to the above examples, it will be understood that modifications and variations are encompassed within the spirit and scope of the invention. Accordingly, the invention is limited only by the following claims.

### Example 14

### Lyophilization of Self-Replicating RNA-Lipid Nanoparticle Formulation Materials and Methods Generally

The processes conducted in this example were conducted using lipid nanoparticle compositions that were manufactured according to well-known processes, for example, those described in U.S. application Ser. No. 16/823,212, the contents of which are incorporated by reference for the specific purpose of teaching lipid nanoparticle manufacturing processes. The lipid nanoparticle compositions and the lyophilized products were characterized for several properties. The materials and methods for these characterization processes as well as a general method of manufacturing the lipid nanoparticle compositions that were used for lyophilization experiments are provided in this example.

### Lipid Nanoparticle Manufacture

Lipid nanoparticle formulations used in this example were manufactured by mixing lipids (ionizable cationic lipid (ATX-126): helper lipid:cholesterol:PEG-lipid) in ethanol with RNA dissolved in citrate buffer. The mixed material was instantaneously diluted with Phosphate Buffer. Ethanol was removed by dialysis against phosphate buffer using regenerated cellulose membrane (100 kD MWCO) or by tangential flow filtration (TFF) using modified polyethersulfone (mPES) hollow fiber membranes (100 kD MWCO). Once the ethanol was completely removed, the buffer was exchanged with HEPES (4-(2-hydroxyethyl)-1-piperazineethanesulfonic acid) buffer containing 10-300 (for example, 40-60) mM NaCl and 5-15% sucrose, pH 7.3. The formulation was concentrated followed by 0.2 μm filtration using PES filters. The RNA concentration in the formulation was then measured by RiboGreen fluorimetric assay, and the concentration was adjusted to a final desired concentration by diluting with HEPES buffer containing 10-100 (for example 40-60) mM NaCl, 0-15% sucrose, pH 7.2-8.5 containing glycerol. If not used immediately for further studies, the final formulation was then filtered through a 0.2 μm filter and filled into glass vials, stoppered, capped and placed at −70±5° C. The lipid nanoparticles formulations were characterized for their pH and osmolality. Lipid Content and RNA content were measured by high performance liquid chromatography (HPLC), and mRNA integrity by was measured by fragment analyzer.

### Dynamic Light Scattering (DLS)

The average particle size (z) and polydispersity index (PDI) of lipid nanoparticle formulations used in the Examples was measured by dynamic light scattering on a Malvern Zetasizer Nano ZS (United Kingdom).

### RiboGreen Assay

The encapsulation efficiency of the lipid nanoparticle formulations was characterized using the RiboGreen fluorometric assay. RiboGreen is a proprietary fluorescent dye (Molecular Probes/Invitrogen a division of Life Technologies, now part of Thermo Fisher Scientific of Eugene, Oregon, United States) that is used in the detection and quantification of nucleic acids, including both RNA and DNA. In its free form, RiboGreen exhibits little fluorescence and possesses a negligible absorbance signature. When bound to nucleic acids, the dye fluoresces with an intensity that is several orders of magnitude greater than the unbound form. The fluorescence can be then be detected by a sensor (fluorimeter) and the nucleic acid can be quantified.

### Lyophilization Process

Self-Replicating RNAs (aka Replicon RNA) are typically larger than the average mRNA, and tests were designed to determine whether self-replicating RNA lipid nanoparticle formulations could be successfully lyophilized. The quality of lyophilized lipid nanoparticle formulations was assessed by analyzing the formulations post-lyophilization and comparing this to the lipid nanoparticle formulation prior to lyophilization as well as after a conventional freeze/thaw cycle (i.e., frozen at ˜-70° C. then allowed to thaw at room temperature).

The analysis of the lipid nanoparticle formulations included the analysis of particle size and polydispersity (PDI) and encapsulation efficiency (% Encap). The particle size post-lyophilization was compared to the particle size pre-lyophilization and the difference can be reported as a delta (6). The various compositions tested were screened as to whether a threshold of properties was met including minimal particle size increase (6<10 nm), the maintenance of PDI (<0.2), and maintenance of high encapsulation efficiency (>85%).

The lipid nanoparticle formulations were prepared as described above, with self-replicating RNA (SEQ ID NO: 125). The resulting lipid nanoparticle formulation was then processed with a buffer exchange to form a prelyophilization suspension having a concentration of 0.05 to 2.0 mg/mL self-replicating RNA, 0.01 to 0.05 M potassium sorbate, 0.01 to 0.10% w/v Poloxamer 188 (Kolliphor®), 14 to 18% w/v sucrose, 25 to 75 mM NaCl, and 15 to 25 mM pH 8.0 Tris buffer. The prelyophilization formulation was then lyophilized in a Millrock Revo Freeze Dryer (Model No. RV85S4), using aliquots of 2.0 mL of suspension and the lyophilization cycle provided in Table 10 below. The lyophilized formulations of this example were then applied to the studies of Example 13 above as “LYO-LNP”.

TABLE 10
Lyophilization Cycle for Self-Replicating
RNA-Lipid Nanoparticle Formulation
Freeze drying cycle

| | shelf | step | chamber |
|---|---|---|---|
| | temperature | duration | vacuum |
| Step | (° C., ±2° C.) | (h:min) | (mbar) |
| Initial Freezing | −50 | 4:00 | atmosphere |
| Evacuation | −50 | 00:30-01:45 | from atmosph. |
| | | | pressure to 0.05 |
| Primary drying | −50 → 0 | 63:00 | 0.05 |
| (ramp down) | | | |
| Secondary drying | 0 → +25 | 39:30 | 0.05 |
| (ramp up) | | | |
| Backfill with N₂ and | 25 | 00:10-00:20 | 700 ± 50 |
| stoppering | | | |
| Aeration with air | 5 | 00:10-00:20 | atmosphere |

The lyophilized particles prepared following the methods described above were reconstituted in 2 mL of water and characterized using DLS and RiboGreen. The results provided in Table 11 below show that the lyophilized compositions were found to produce lyophilized lipid nanoparticle formulations with adequate size, polydispersity, and delta values (˜5.3 nm) upon reconstitution.

TABLE 11
Self-Replicating RNA-Lipid Nanoparticle
Characteristics Pre- and Post-LYO

| | Average | | |
|---|---|---|---|
| | Particle | | |
| | Size (nm) | PDI | encap (%) |
| Pre-LYO | 76.3 | 0.129 | 97 |
| Post-LYO | 81.6 | 0.152 | 93 |

1. A nucleic acid molecule comprising:
(i) a first polynucleotide encoding one or more viral replication proteins, wherein the first polynucleotide is codon-optimized as compared to a wild-type polynucleotide encoding the one or more viral replication proteins, and wherein the first polynucleotide comprises a sequence having at least 80% identity to the sequence of SEQ ID NO:72; and
(ii) a second polynucleotide comprising a transgene encoding an antigenic protein or a fragment thereof, wherein the antigenic protein is a coronavirus protein.

2.-8. (canceled)

9. The nucleic acid molecule of claim 1, further comprising
(a) a 5′ untranslated region (UTR); or
(b) a 5′ untranslated region (UTR) comprising an alphavirus 5′ UTR sequence; or
(c) 5′ untranslated region (UTR) comprising a sequence having at least 80% identity to the sequence of SEQ ID NO:73, SEQ ID NO:74, or SEQ ID NO:75; or
(d) a 3′ untranslated region (UTR); or
(e) a 3′ untranslated region (UTR) comprising an alphavirus 3′ UTR sequence; or
(f) a 3′ untranslated region (UTR) comprising an alphavirus 3′ UTR sequence, wherein the 3′ UTR further comprises a poly-A sequence.

10.-19. (canceled)

20. The nucleic acid molecule of claim 1, wherein the antigenic protein is a SARS-CoV-2 protein, a SARS-CoV-2 spike glycoprotein, or a variant SARS-CoV-2 spike glycoprotein.

21.-28. (canceled)

29. The nucleic acid molecule of claim 1, wherein the first polynucleotide is located 5′ of the second polynucleotide.

30. The nucleic acid molecule of claim 29, further comprising
(a) an intergenic region located between the first polynucleotide and the second polynucleotide; or
(b) an intergenic region located between the first polynucleotide and the second polynucleotide, wherein the intergenic region comprises a sequence having at least 85% identity to the sequence of SEQ ID NO:77.

31. (canceled)

32. The nucleic acid molecule of claim 1, wherein the nucleic acid molecule is
(a) a DNA molecule; or
(b) an RNA molecule, wherein T is substituted with U.

33. The nucleic acid molecule of claim 32, wherein the DNA molecule further comprises a promoter, wherein the promoter is a T7 promoter, a T3 promoter, or an SP6 promoter.

34. (canceled)

35. (canceled)

36. The nucleic acid molecule of claim 32, wherein the RNA molecule is a self-replicating RNA molecule.

37. The nucleic acid molecule of claim 32, wherein the RNA molecule further comprises a 5′ cap having a Cap 1 structure, a Cap 1 (^{m6}A) structure, a Cap 2 structure, or a Cap 0 structure.

38.-75. (canceled)

76. A composition comprising the nucleic acid molecule of claim 1 and a lipid formulation.

77.-80. (canceled)

81. The composition of claim 76, wherein the lipid formulation is a lipid nanoparticle.

82.-87. (canceled)

88. The composition of claim 76, wherein the lipid formulation comprises an ionizable cationic lipid.

89. The composition of claim 88, wherein the ionizable cationic lipid has a structure of Formula I: 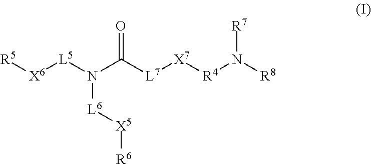
or a pharmaceutically acceptable salt or solvate thereof, wherein R⁵ and R⁶ are each independently selected from the group consisting of a linear or branched C₁-C₃₁ alkyl, C₂-C₃₁ alkenyl or C₂-C₃₁ alkynyl and cholesteryl; L⁵ and L⁶ are each independently selected from the group consisting of a linear C₁-C₂₀ alkyl and C₂-C₂₀ alkenyl; X⁵ is —C(O)O—, whereby —C(O)O—R⁶ is formed or —OC(O)— whereby —OC(O)—R⁶ is formed; X⁶ is —C(O)O— whereby —C(O)O—R⁵ is formed or —OC(O)— whereby —OC(O)—R⁵ is formed; X⁷ is S or O; L⁷ is absent or lower alkyl; R⁴ is a linear or branched C₁-C₆ alkyl; and R⁷ and R⁸ are each independently selected from the group consisting of a hydrogen and a linear or branched C₁-C₆ alkyl.

90. (canceled)

91. The composition of claim 88, wherein the ionizable cationic lipid has a structure of: 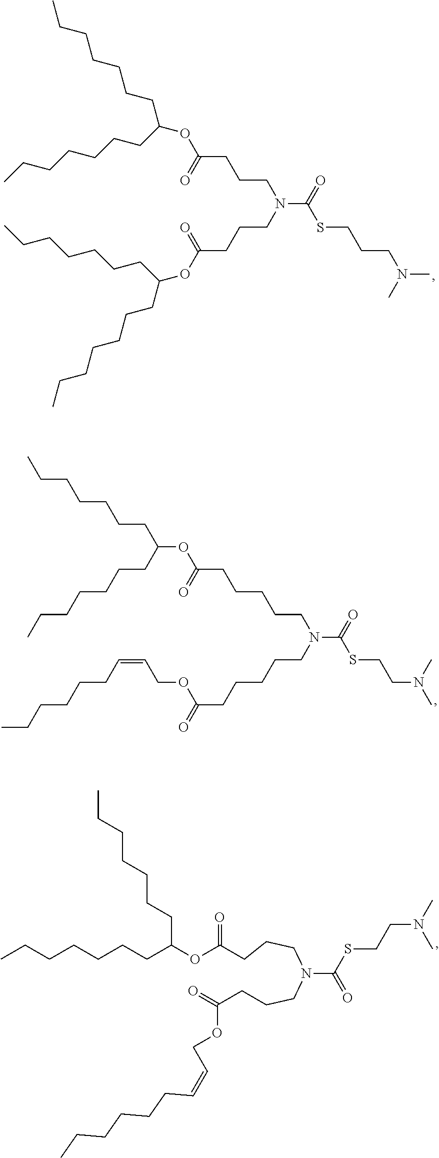
or a pharmaceutically acceptable salt thereof.

92. The composition of claim 76, wherein the lipid formulation encapsulates the nucleic acid molecule or is complexed to the nucleic acid molecule.

93. (canceled)

94. The composition of claim 76, wherein the lipid formulation comprises
(a) a helper lipid;
(b) a phospholipid;
(c) a polyethylene glycol (PEG)-lipid conjugate; or
(d) any combination thereof.

95. (canceled)

96. The composition of claim 94, wherein the phospholipid is selected from dioleoylphosphatidyl ethanolamine (DOPE), dimyristoylphosphatidyl choline (DMPC), distearoylphosphatidyl choline (DSPC), dimyristoylphosphatidyl glycerol (DMPG), dipalmitoyl phosphatidylcholine (DPPC), and phosphatidylcholine (PC).

97.-101. (canceled)

102. The composition of claim 76, wherein the lipid portion of the lipid formulation comprises about 40 mol % to about 60 mol % of t+e an ionizable cationic lipid, about 4 mol % to about 16 mol % DSPC, about 30 mol % to about 47 mol % cholesterol, and about 0.5 mol % to about 3 mol % PEG2000-DMG.

103. (canceled)

104. (canceled)

105. The composition of claim 76, wherein the composition has a total lipid:nucleic acid molecule weight ratio of about 50:1 to about 10:1.

106.-109. (canceled)

110. The composition of claim 76, wherein the composition comprises
(a) a HEPES or TRIS buffer at a pH of about 7.0 to about 8.5;
(b) a HEPES or TRIS buffer at a concentration of about 7 mg/mL to about 15 mg/mL;
(c) about 2.0 mg/mL to about 4.0 mg/mL of NaCl;
(d) one or more cryoprotectants;
(e) one or more cryoprotectants selected from sucrose, glycerol, or a combination of sucrose and glycerol; or
(f) any combination thereof.

111.-115. (canceled)

116. The composition of claim 76, wherein
(a) the composition is a lyophilized composition; or
(b) the composition is a lyophilized composition comprising one or more lyoprotectants: or
(c) the composition is a lyophilized composition comprising a poloxamer, potassium sorbate, sucrose, or any combination thereof.

117.-119. (canceled)

120. The composition of claim 116, wherein the lyophilized composition comprises
(a) about 0.01% to about 1.0% w/w of the nucleic acid molecule;
(b) about 1.0% to about 5.0% w/w lipids;
(c) about 0.5% to about 2.5% w/w of TRIS buffer;
(d) about 0.75% to about 2.75% w/w of NaCl;
(e) about 85% to about 95% w/w of a sugar;
(f) about 0.01% to about 1.0% w/w of a poloxamer;
(g) about 1.0% to about 5.0% w/w of potassium sorbate; or
(h) any combination thereof.

121.-132. (canceled)

133. A method of administering the composition of claim 76 to a subject in need thereof, wherein the composition is lyophilized and is reconstituted prior to administration.

134. A method of preventing or ameliorating COVID-19, comprising administering the composition of claim 76 to a subject in need thereof.

135. (canceled)

136. (canceled)

137. A method of administering a booster dose to a vaccinated subject, comprising administering the composition of claim 76 to a subject who was previously vaccinated against coronavirus.

138. The method of claim 134, wherein
(a) the composition is administered at a dosage of about 0.01 μg to about 1,000 μg of nucleic acid; or
(b) wherein the composition is administered at a dosage of about 1, 2, 5, 7.5, or 10 μg of nucleic acid.

139. (canceled)

140. A method of inducing an immune response against a coronavirus in a subject comprising:
administering to the subject an effective amount of the nucleic acid molecule of claim 1.

141. (canceled)

142. A method of inducing an immune response in a subject comprising:
administering to the subject an effective amount of the composition of claim 76.

143.-145. (canceled)


SEQUENCE LISTING (129 sequences)

SEQ ID NO: 1 - DNA, 18 bp, synthetic construct
  misc_feature 1..18: note = Synthetic polynucleotide
  source 1..18: mol_type = other DNA
  modified_base 15: mod_base = OTHER; note = uracil
gaggaaacttaagatggg

SEQ ID NO: 2-4 - skipped (3 entries)

SEQ ID NO: 5 - RNA, 129 nt, synthetic construct
  misc_feature 1..129: note = Synthetic polynucleotide
  source 1..129: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatag

SEQ ID NO: 6 - RNA, 28 nt, synthetic construct
  misc_feature 1..28: note = Synthetic polynucleotide
  source 1..28: mol_type = other RNA
attattacatcaaaacaaaaagccgcca

SEQ ID NO: 7 - RNA, 245 nt, synthetic construct
  misc_feature 1..245: note = Synthetic polynucleotide
  source 1..245: mol_type = other RNA
cttaagggggcgctgcctacggaggtggcagccatctccttctcggcatcaagcttaccatggtgccccaggccctgctcttggtcccgctgctggtgttccccctctgcttcggcaagttccccatctacaccatccccgacaagctggggccgtggagccccatcgacatccaccacctgtcctgccccaacaacctcgtggtcgaggacgagggctgcaccaacctgagcgggttctcctac

SEQ ID NO: 8 - RNA, 177 nt, synthetic construct
  misc_feature 1..177: note = Synthetic polynucleotide
  source 1..177: mol_type = other RNA
tgagtgtcgtacagcctccaggcccccccctcccgggagagccatagtggtctgcggaaccggtgagtacaccggaattgccgggaagactgggtcctttcttggataaacccactctatgcccggccatttgggcgtgcccccgcaagactgctagccgagtagtgttgggttgcg

SEQ ID NO: 9 - RNA, 89 nt, synthetic construct
  misc_feature 1..89: note = Synthetic polynucleotide
  source 1..89: mol_type = other RNA
aattattggttaaagaagtatattagtgctaatttccctccgtttgtcctagcttttctcttctgtcaaccccacacgcctttggcaca

SEQ ID NO: 10 - RNA, 569 nt, synthetic construct
  misc_feature 1..569: note = Synthetic polynucleotide
  source 1..569: mol_type = other RNA
ctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacgtgtgtttagtcgaggttaaaaaacgtctaggccccccgaaccacggggacgtggttttcctttgaaaaacacgatgataat

SEQ ID NO: 11 - RNA, 44 nt, synthetic construct
  misc_feature 1..44: note = Synthetic polynucleotide
  source 1..44: mol_type = other RNA
gtcagctttcaaactctttgtttcttgtttgttgattgagaata

SEQ ID NO: 12 - RNA, 54 nt, synthetic construct
  misc_feature 1..54: note = Synthetic polynucleotide
  source 1..54: mol_type = other RNA
ctctcgcctgagaaaaaaaatccacgaaccaatttctcagcaaccagcagcacg

SEQ ID NO: 13 - RNA, 52 nt, synthetic construct
  misc_feature 1..52: note = Synthetic polynucleotide
  source 1..52: mol_type = other RNA
acctgtgagggttcgaaggaagtagcagtgttttttgttcctagaggaagag

SEQ ID NO: 14 - RNA, 71 nt, synthetic construct
  misc_feature 1..71: note = Synthetic polynucleotide
  source 1..71: mol_type = other RNA
acacagaaacattcgcaaaaacaaaatcccagtatcaaaattcttctctttttttcatatttcgcaaagac

SEQ ID NO: 15 - RNA, 52 nt, synthetic construct
  misc_feature 1..52: note = Synthetic polynucleotide
  source 1..52: mol_type = other RNA
cagaaaaatttgctacattgtttcacaaacttcaaatattattcatttattt

SEQ ID NO: 16 - RNA, 158 nt, synthetic construct
  misc_feature 1..158: note = Synthetic polynucleotide
  source 1..158: mol_type = other RNA
ctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacat

SEQ ID NO: 17 - RNA, 166 nt, synthetic construct
  misc_feature 1..166: note = Synthetic polynucleotide
  source 1..166: mol_type = other RNA
tgcaaggctggccggaagcccttgcctgaaagcaagatttcagcctggaagagggcaaagtggacgggagtggacaggagtggatgcgataagatgtggtttgaagctgatgggtgccagccctgcattgctgagtcaatcaataaagagctttcttttgacccat

SEQ ID NO: 18 - RNA, 143 nt, synthetic construct
  misc_feature 1..143: note = Synthetic polynucleotide
  source 1..143: mol_type = other RNA
acgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgca

SEQ ID NO: 19 - RNA, 220 nt, synthetic construct
  misc_feature 1..220: note = Synthetic polynucleotide
  source 1..220: mol_type = other RNA
tagagcggcaaaccctagctacactccatagctagtttcttttttttttgtttttttttttttttttttttttttttttttttttttttcctttcttttccttctttttttcctcttttcttggtggctccatcttagccctagtcacggctagctgtgaaaggtccgtgagccgcatgactgcagagagtgccgtaactggtctctctgcagatcatgt

SEQ ID NO: 20 - RNA, 170 nt, synthetic construct
  misc_feature 1..170: note = Synthetic polynucleotide
  source 1..170: mol_type = other RNA
acacatcacaaccacaaccttctcaggctaccctgagaaaaaaagacatgaagactcaggactcatcttttctgttggtgtaaaatcaacaccctaaggaacacaaatttctttaaacatttgacttcttgtctctgtgctgcaattaataaaaaatggaaagaatctac

SEQ ID NO: 21 - RNA, 110 nt, synthetic construct
  misc_feature 1..110: note = Synthetic polynucleotide
  source 1..110: mol_type = other RNA
gctggagcctcggtagccgttcctcctgcccgctgggcctcccaacgggccctcctcccctccttgcaccggcccttcctggtctttgaataaagtctgagtgggcagca

SEQ ID NO: 22 - RNA, 123 nt, synthetic construct
  misc_feature 1..123: note = Synthetic polynucleotide
  source 1..123: mol_type = other RNA
tagtgcagtcactggcacaacgcgttgcccggtaagccaatcgggtatacacggtcgtcatactgcagacagggttcttctactttgcaagatagtctagagtagtaaaataaatagtataag

SEQ ID NO: 23-24 - skipped (2 entries)

SEQ ID NO: 25 - RNA, 21 nt, synthetic construct
  misc_feature 1..21: note = Synthetic polynucleotide
  source 1..21: mol_type = other RNA
cacaaagagtaaagaagaaca

SEQ ID NO: 26 - RNA, 21 nt, synthetic construct
  misc_feature 1..21: note = Synthetic polynucleotide
  source 1..21: mol_type = other RNA
aacactaaaagtagaagaaaa

SEQ ID NO: 27 - RNA, 21 nt, synthetic construct
  misc_feature 1..21: note = Synthetic polynucleotide
  source 1..21: mol_type = other RNA
ctcagaaagataagatcagcc

SEQ ID NO: 28 - RNA, 21 nt, synthetic construct
  misc_feature 1..21: note = Synthetic polynucleotide
  source 1..21: mol_type = other RNA
aaccaatcgaaagaaaccaaa

SEQ ID NO: 29 - RNA, 21 nt, synthetic construct
  misc_feature 1..21: note = Synthetic polynucleotide
  source 1..21: mol_type = other RNA
ctctaatcaccaggagtaaaa

SEQ ID NO: 30 - RNA, 21 nt, synthetic construct
  misc_feature 1..21: note = Synthetic polynucleotide
  source 1..21: mol_type = other RNA
gagagagatcttaacaaaaaa

SEQ ID NO: 31 - RNA, 21 nt, synthetic construct
  misc_feature 1..21: note = Synthetic polynucleotide
  source 1..21: mol_type = other RNA
tgtgtaacaacaacaacaaca

SEQ ID NO: 32 - RNA, 21 nt, synthetic construct
  misc_feature 1..21: note = Synthetic polynucleotide
  source 1..21: mol_type = other RNA
ccgcagtaggaagagaaagcc

SEQ ID NO: 33 - RNA, 21 nt, synthetic construct
  misc_feature 1..21: note = Synthetic polynucleotide
  source 1..21: mol_type = other RNA
aaaaaaaaaagaaatcataaa

SEQ ID NO: 34 - RNA, 21 nt, synthetic construct
  misc_feature 1..21: note = Synthetic polynucleotide
  source 1..21: mol_type = other RNA
gagagaagaaagaagaagacg

SEQ ID NO: 35 - RNA, 21 nt, synthetic construct
  misc_feature 1..21: note = Synthetic polynucleotide
  source 1..21: mol_type = other RNA
caattaaaaatacttaccaaa

SEQ ID NO: 36 - RNA, 21 nt, synthetic construct
  misc_feature 1..21: note = Synthetic polynucleotide
  source 1..21: mol_type = other RNA
gcaaacagagtaagcgaaacg

SEQ ID NO: 37 - RNA, 21 nt, synthetic construct
  misc_feature 1..21: note = Synthetic polynucleotide
  source 1..21: mol_type = other RNA
gcgaagaagacgaacgcaaag

SEQ ID NO: 38 - RNA, 21 nt, synthetic construct
  misc_feature 1..21: note = Synthetic polynucleotide
  source 1..21: mol_type = other RNA
ttaggactgtattgactggcc

SEQ ID NO: 39 - RNA, 21 nt, synthetic construct
  misc_feature 1..21: note = Synthetic polynucleotide
  source 1..21: mol_type = other RNA
atcatcggaattcggaaaaag

SEQ ID NO: 40 - RNA, 21 nt, synthetic construct
  misc_feature 1..21: note = Synthetic polynucleotide
  source 1..21: mol_type = other RNA
aaaacaaaagttaaagcagac

SEQ ID NO: 41 - RNA, 21 nt, synthetic construct
  misc_feature 1..21: note = Synthetic polynucleotide
  source 1..21: mol_type = other RNA
tttatctcaaataagaaggca

SEQ ID NO: 42 - RNA, 21 nt, synthetic construct
  misc_feature 1..21: note = Synthetic polynucleotide
  source 1..21: mol_type = other RNA
ggtggggaggtgagatttctt

SEQ ID NO: 43 - RNA, 21 nt, synthetic construct
  misc_feature 1..21: note = Synthetic polynucleotide
  source 1..21: mol_type = other RNA
tgattaggaaactacaaagcc

SEQ ID NO: 44 - RNA, 21 nt, synthetic construct
  misc_feature 1..21: note = Synthetic polynucleotide
  source 1..21: mol_type = other RNA
catttttcaatttcataaaac

SEQ ID NO: 45 - RNA, 21 nt, synthetic construct
  misc_feature 1..21: note = Synthetic polynucleotide
  source 1..21: mol_type = other RNA
ttacttttaagcccaacaaaa

SEQ ID NO: 46 - RNA, 21 nt, synthetic construct
  misc_feature 1..21: note = Synthetic polynucleotide
  source 1..21: mol_type = other RNA
ggcgtgtgtgtgtgttgttga

SEQ ID NO: 47 - RNA, 21 nt, synthetic construct
  misc_feature 1..21: note = Synthetic polynucleotide
  source 1..21: mol_type = other RNA
gtggtgaaggggaaggtttag

SEQ ID NO: 48 - RNA, 21 nt, synthetic construct
  misc_feature 1..21: note = Synthetic polynucleotide
  source 1..21: mol_type = other RNA
ttgtttttttttggtttggtt

SEQ ID NO: 49 - DNA, 44 bp, synthetic construct
  misc_feature 1..44: note = Synthetic polynucleotide
  source 1..44: mol_type = other DNA
atgggcggcgcatgagagaagcccagaccaattacctacccaaa

SEQ ID NO: 50 - DNA, 7482 bp, synthetic construct
  misc_feature 1..7482: note = Synthetic polynucleotide
  source 1..7482: mol_type = other DNA
atggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagaccgtgctgaccgccaggaacatcggcctgtgcagcagcgacgtgatggagaggagccggagaggcatgagcatcctgaggaagaaatacctgaagcccagcaacaacgtgctgttcagcgtgggcagcaccatctaccacgagaagagggacctgctcaggagctggcacctgcccagcgtgttccacctgaggggcaagcagaactacacctgcaggtgcgagaccatcgtgagctgcgacggctacgtggtgaagaggatcgccatcagccccggcctgtacggcaagcccagcggctacgccgctacaatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctcgtgggcctgaaccagaggatcgtggtcaacggcaggacccagaggaacaccaacacaatgaagaactacctgctgcccgtggtggcccaggctttcgccaggtgggccaaggagtacaaggaggaccaggaagacgagaggcccctgggcctgagggacaggcagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggaacacaaggagcccagcccactgatcaccgccgaggacgtgcaggaggccaagtgcgctgccgacgaggccaaggaggtgagggaggccgaggaactgagggccgccctgccacccctggctgccgacgtggaggaacccaccctggaagccgacgtggacctgatgctgcaggaggccggcgccggaagcgtggagacacccaggggcctgatcaaggtgaccagctacgacggcgaggacaagatcggcagctacgccgtgctgagcccacaggccgtgctgaagtccgagaagctgagctgcatccacccactggccgagcaggtgatcgtgatcacccacagcggcaggaagggcaggtacgccgtggagccctaccacggcaaggtggtcgtgcccgagggccacgccatccccgtgcaggacttccaggccctgagcgagagcgccaccatcgtgtacaacgagagggagttcgtgaacaggtacctgcaccatatcgccacccacggcggagccctgaacaccgacgaggaatactacaagaccgtgaagcccagcgagcacgacggcgagtacctgtacgacatcgacaggaagcagtgcgtgaagaaagagctggtgaccggcctgggactgaccggcgagctggtggacccacccttccacgagttcgcctacgagagcctgaggaccagacccgccgctccctaccaggtgcccaccatcggcgtgtacggcgtgcccggcagcggaaagagcggcatcatcaagagcgccgtgaccaagaaagacctggtggtcagcgccaagaaagagaactgcgccgagatcatcagggacgtgaagaagatgaaaggcctggacgtgaacgcgcgcaccgtggacagcgtgctgctgaacggctgcaagcaccccgtggagaccctgtacatcgacgaggccttcgcttgccacgccggcaccctgagggccctgatcgccatcatcaggcccaagaaagccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtcgtgagcaccctgttctacgacaagaaaatgaggaccaccaaccccaaggagaccaaaatcgtgatcgacaccacaggcagcaccaagcccaagcaggacgacctgatcctgacctgcttcaggggctgggtgaagcagctgcagatcgactacaagggcaacgagatcatgaccgccgctgccagccagggcctgaccaggaagggcgtgtacgccgtgaggtacaaggtgaacgagaacccactgtacgctcccaccagcgagcacgtgaacgtgctgctgaccaggaccgaggacaggatcgtgtggaagaccctggccggcgacccctggatcaagaccctgaccgccaagtaccccggcaacttcaccgccaccatcgaagagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccacagagcagtggaacaccgtggactacttcgagaccgacaaggcccacagcgccgagatcgtgctgaaccagctgtgcgtgaggttcttcggcctggacctggacagcggcctgttcagcgcccccaccgtgccactgagcatcaggaacaaccactgggacaacagccccagcccaaacatgtacggcctgaacaaggaggtggtcaggcagctgagcaggcggtacccacagctgcccagggccgtggccaccggcagggtgtacgacatgaacaccggcaccctgaggaactacgaccccaggatcaacctggtgcccgtgaacaggcggctgccccacgccctggtgctgcaccacaacgagcacccacagagcgacttcagctccttcgtgagcaagctgaaaggcaggaccgtgctggtcgtgggcgagaagctgagcgtgcccggcaagatggtggactggctgagcgacaggcccgaggccaccttccgggccaggctggacctcggcatccccggcgacgtgcccaagtacgacatcatcttcgtgaacgtcaggaccccatacaagtaccaccattaccagcagtgcgaggaccacgccatcaagctgagcatgctgaccaagaaggcctgcctgcacctgaaccccggaggcacctgcgtgagcatcggctacggctacgccgacagggccagcgagagcatcattggcgccatcgccaggctgttcaagttcagcagggtgtgcaaacccaagagcagcctggaggaaaccgaggtgctgttcgtgttcatcggctacgaccggaaggccaggacccacaacccctacaagctgagcagcaccctgacaaacatctacaccggcagcaggctgcacgaggccggctgcgcccccagctaccacgtggtcaggggcgatatcgccaccgccaccgagggcgtgatcatcaacgctgccaacagcaagggccagcccggaggcggagtgtgcggcgccctgtacaagaagttccccgagagcttcgacctgcagcccatcgaggtgggcaaggccaggctggtgaagggcgccgctaagcacatcatccacgccgtgggccccaacttcaacaaggtgagcgaggtggaaggcgacaagcagctggccgaagcctacgagagcatcgccaagatcgtgaacgacaataactacaagagcgtggccatcccactgctcagcaccggcatcttcagcggcaacaaggacaggctgacccagagcctgaaccacctgctcaccgccctggacaccaccgatgccgacgtggccatctactgcagggacaagaagtgggagatgaccctgaaggaggccgtggccaggcgggaggccgtggaagagatctgcatcagcgacgactccagcgtgaccgagcccgacgccgagctggtgagggtgcaccccaagagctccctggccggcaggaagggctacagcaccagcgacggcaagaccttcagctacctggagggcaccaagttccaccaggccgctaaggacatcgccgagatcaacgctatgtggcccgtggccaccgaggccaacgagcaggtgtgcatgtacatcctgggcgagagcatgtccagcatcaggagcaagtgccccgtggaggaaagcgaggccagcacaccacccagcaccctgccctgcctgtgcatccacgctatgacacccgagagggtgcagcggctgaaggccagcaggcccgagcagatcaccgtgtgcagctccttcccactgcccaagtacaggatcaccggcgtgcagaagatccagtgcagccagcccatcctgttcagcccaaaggtgcccgcctacatccaccccaggaagtacctggtggagaccccacccgtggacgagacacccgagccaagcgccgagaaccagagcaccgagggcacacccgagcagccacccctgatcaccgaggacgagacaaggacccggaccccagagcccatcattatcgaggaagaggaagaggacagcatcagcctgctgagcgacggccccacccaccaggtgctgcaggtggaggccgacatccacggcccacccagcgtgtccagctccagctggagcatcccacacgccagcgacttcgacgtggacagcctgagcatcctggacaccctggagggcgccagcgtgacctccggcgccaccagcgccgagaccaacagctacttcgccaagagcatggagttcctggccaggcccgtgccagctcccaggaccgtgttcaggaacccaccccacccagctcccaggaccaggaccccaagcctggctcccagcagggcctgcagcaggaccagcctggtgagcaccccacccggcgtgaacagggtgatcaccagggaggaactggaggccctgacacccagcaggacccccagcaggtccgtgagcaggactagtctggtgtccaacccacccggcgtgaacagggtgatcaccagggaggaattcgaggccttcgtggcccagcaacagagacggttcgacgccggcgcctacatcttcagcagcgacaccggccagggacacctgcagcaaaagagcgtgaggcagaccgtgctgagcgaggtggtgctggagaggaccgagctggaaatcagctacgcccccaggctggaccaggagaaggaggaactgctcaggaagaaactgcagctgaaccccaccccagccaacaggagcaggtaccagagcaggaaggtggagaacatgaaggccatcaccgccaggcggatcctgcagggcctgggacactacctgaaggccgagggcaaggtggagtgctacaggaccctgcaccccgtgccactgtacagctccagcgtgaacagggccttctccagccccaaggtggccgtggaggcctgcaacgctatgctgaaggagaacttccccaccgtggccagctactgcatcatccccgagtacgacgcctacctggacatggtggacggcgccagctgctgcctggacaccgccagcttctgccccgccaagctgaggagcttccccaagaaacacagctacctggagcccaccatcaggagcgccgtgcccagcgccatccagaacaccctgcagaacgtgctggccgctgccaccaagaggaactgcaacgtgacccagatgagggagctgcccgtgctggacagcgctgccttcaacgtggagtgcttcaagaaatacgcctgcaacaacgagtactgggagaccttcaaggagaaccccatcaggctgaccgaagagaacgtggtgaactacatcaccaagctgaagggccccaaggccgctgccctgttcgctaagacccacaacctgaacatgctgcaggacatcccaatggacaggttcgtgatggacctgaagagggacgtgaaggtgacacccggcaccaagcacaccgaggagaggcccaaggtgcaggtgatccaggccgctgacccactggccaccgcctacctgtgcggcatccacagggagctggtgaggcggctgaacgccgtgctgctgcccaacatccacaccctgttcgacatgagcgccgaggacttcgacgccatcatcgccgagcacttccagcccggcgactgcgtgctggagaccgacatcgccagcttcgacaagagcgaggatgacgctatggccctgaccgctctgatgatcctggaggacctgggcgtggacgccgagctgctcaccctgatcgaggctgccttcggcgagatcagctccatccacctgcccaccaagaccaagttcaagttcggcgctatgatgaaaagcggaatgttcctgaccctgttcgtgaacaccgtgatcaacattgtgatcgccagcagggtgctgcgggagaggctgaccggcagcccctgcgctgccttcatcggcgacgacaacatcgtgaagggcgtgaaaagcgacaagctgatggccgacaggtgcgccacctggctgaacatggaggtgaagatcatcgacgccgtggtgggcgagaaggccccctacttctgcggcggattcatcctgtgcgacagcgtgaccggcaccgcctgcagggtggccgaccccctgaagaggctgttcaagctgggcaagccactggccgctgacgatgagcacgacgatgacaggcggagggccctgcacgaggaaagcaccaggtggaacagggtgggcatcctgagcgagctgtgcaaggccgtggagagcaggtacgagaccgtgggcaccagcatcatcgtgatggctatgaccacactggccagctccgtcaagagcttctcctacctgaggggggcccctataactctctacggctaa

SEQ ID NO: 51 - protein, 2493 aa, synthetic construct
  REGION 1..2493: note = Synthetic polypeptide
MEKVHVDIEEDSPFLRALQRSFPQFEVEAKQVTDNDHANARAFSHLASKLIETEVDPSDTILDIGSAPARRMYSKHKYHCICPMRCAEDPDRLYKYATKLKKNCKEITDKELDKKMKELAAVMSDPDLETETMCLHDDESCRYEGQVAVYQDVYAVDGPTSLYHQANKGVRVAYWIGFDTTPFMFKNLAGAYPSYSTNWADETVLTARNIGLCSSDVMERSRRGMSILRKKYLKPSNNVLFSVGSTIYHEKRDLLRSWHLPSVFHLRGKQNYTCRCETIVSCDGYVVKRIAISPGLYGKPSGYAATMHREGFLCCKVTDTLNGERVSFPVCTYVPATLCDQMTGILATDVSADDAQKLLVGLNQRIVVNGRTQRNTNTMKNYLLPVVAQAFARWAKEYKEDQEDERPLGLRDRQLVMGCCWAFRRHKITSIYKRPDTQTIIKVNSDFHSFVLPRIGSNTLEIGLRTRIRKMLEEHKEPSPLITAEDVQEAKCAADEAKEVREAEELRAALPPLAADVEEPTLEADVDLMLQEAGAGSVETPRGLIKVTSYDGEDKIGSYAVLSPQAVLKSEKLSCIHPLAEQVIVITHSGRKGRYAVEPYHGKVVVPEGHAIPVQDFQALSESATIVYNEREFVNRYLHHIATHGGALNTDEEYYKTVKPSEHDGEYLYDIDRKQCVKKELVTGLGLTGELVDPPFHEFAYESLRTRPAAPYQVPTIGVYGVPGSGKSGIIKSAVTKKDLVVSAKKENCAEIIRDVKKMKGLDVNARTVDSVLLNGCKHPVETLYIDEAFACHAGTLRALIAIIRPKKAVLCGDPKQCGFFNMMCLKVHFNHEICTQVFHKSISRRCTKSVTSVVSTLFYDKKMRTTNPKETKIVIDTTGSTKPKQDDLILTCFRGWVKQLQIDYKGNEIMTAAASQGLTRKGVYAVRYKVNENPLYAPTSEHVNVLLTRTEDRIVWKTLAGDPWIKTLTAKYPGNFTATIEEWQAEHDAIMRHILERPDPTDVFQNKANVCWAKALVPVLKTAGIDMTTEQWNTVDYFETDKAHSAEIVLNQLCVRFFGLDLDSGLFSAPTVPLSIRNNHWDNSPSPNMYGLNKEVVRQLSRRYPQLPRAVATGRVYDMNTGTLRNYDPRINLVPVNRRLPHALVLHHNEHPQSDFSSFVSKLKGRTVLVVGEKLSVPGKMVDWLSDRPEATFRARLDLGIPGDVPKYDIIFVNVRTPYKYHHYQQCEDHAIKLSMLTKKACLHLNPGGTCVSIGYGYADRASESIIGAIARLFKFSRVCKPKSSLEETEVLFVFIGYDRKARTHNPYKLSSTLTNIYTGSRLHEAGCAPSYHVVRGDIATATEGVIINAANSKGQPGGGVCGALYKKFPESFDLQPIEVGKARLVKGAAKHIIHAVGPNFNKVSEVEGDKQLAEAYESIAKIVNDNNYKSVAIPLLSTGIFSGNKDRLTQSLNHLLTALDTTDADVAIYCRDKKWEMTLKEAVARREAVEEICISDDSSVTEPDAELVRVHPKSSLAGRKGYSTSDGKTFSYLEGTKFHQAAKDIAEINAMWPVATEANEQVCMYILGESMSSIRSKCPVEESEASTPPSTLPCLCIHAMTPERVQRLKASRPEQITVCSSFPLPKYRITGVQKIQCSQPILFSPKVPAYIHPRKYLVETPPVDETPEPSAENQSTEGTPEQPPLITEDETRTRTPEPIIIEEEEEDSISLLSDGPTHQVLQVEADIHGPPSVSSSSWSIPHASDFDVDSLSILDTLEGASVTSGATSAETNSYFAKSMEFLARPVPAPRTVFRNPPHPAPRTRTPSLAPSRACSRTSLVSTPPGVNRVITREELEALTPSRTPSRSVSRTSLVSNPPGVNRVITREEFEAFVAQQQRRFDAGAYIFSSDTGQGHLQQKSVRQTVLSEVVLERTELEISYAPRLDQEKEELLRKKLQLNPTPANRSRYQSRKVENMKAITARRILQGLGHYLKAEGKVECYRTLHPVPLYSSSVNRAFSSPKVAVEACNAMLKENFPTVASYCIIPEYDAYLDMVDGASCCLDTASFCPAKLRSFPKKHSYLEPTIRSAVPSAIQNTLQNVLAAATKRNCNVTQMRELPVLDSAAFNVECFKKYACNNEYWETFKENPIRLTEENVVNYITKLKGPKAAALFAKTHNLNMLQDIPMDRFVMDLKRDVKVTPGTKHTEERPKVQVIQAADPLATAYLCGIHRELVRRLNAVLLPNIHTLFDMSAEDFDAIIAEHFQPGDCVLETDIASFDKSEDDAMALTALMILEDLGVDAELLTLIEAAFGEISSIHLPTKTKFKFGAMMKSGMFLTLFVNTVINIVIASRVLRERLTGSPCAAFIGDDNIVKGVKSDKLMADRCATWLNMEVKIIDAVVGEKAPYFCGGFILCDSVTGTACRVADPLKRLFKLGKPLAADDEHDDDRRRALHEESTRWNRVGILSELCKAVESRYETVGTSIIVMAMTTLASSVKSFSYLRGAPITLYG

SEQ ID NO: 52 - DNA, 44 bp, synthetic construct
  misc_feature 1..44: note = Synthetic polynucleotide
  source 1..44: mol_type = other DNA
cctgaatggactacgacatagtctagtccgccaaggccgccacc

SEQ ID NO: 53 - DNA, 468 bp, synthetic construct
  misc_feature 1..468: note = Synthetic polynucleotide
  source 1..468: mol_type = other DNA
actcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 54 - DNA, 7485 bp, synthetic construct
  misc_feature 1..7485: note = Synthetic polynucleotide
  source 1..7485: mol_type = other DNA
atgcccgagaaggtgcacgtggacatcgaggaggacagccccttcctgagggccctgcagaggagcttcccacagttcgaagtggaggccaagcaggtgaccgacaacgaccacgccaacgccagggccttcagccacctggccagcaagctgatcgagaccgaggtggaccccagcgacaccatcctggacatcggcagcgccccagccaggagaatgtacagcaagcacaagtaccactgcatctgccccatgaggtgcgccgaggaccccgacaggctgtacaagtacgccaccaaactgaagaagaactgcaaggagatcaccgacaaggagctggacaagaaaatgaaggagctggccgccgtgatgagcgaccccgacctggagaccgagacaatgtgcctgcacgacgacgagagctgcaggtacgagggccaggtggccgtctaccaggacgtgtacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagaccgtgctgaccgccaggaacatcggcctgtgcagcagcgacgtgatggagaggagccggagaggcatgagcatcctgaggaagaaatacctgaagcccagcaacaacgtgctgttcagcgtgggcagcaccatctaccacgagaagagggacctgctcaggagctggcacctgcccagcgtgttccacctgaggggcaagcagaactacacctgcaggtgcgagaccatcgtgagctgcgacggctacgtggtgaagaggatcgccatcagccccggcctgtacggcaagcccagcggctacgccgctacaatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctcgtgggcctgaaccagaggatcgtggtcaacggcaggacccagaggaacaccaacacaatgaagaactacctgctgcccgtggtggcccaggctttcgccaggtgggccaaggagtacaaggaggaccaggaagacgagaggcccctgggcctgagggacaggcagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggaacacaaggagcccagcccactgatcaccgccgaggacgtgcaggaggccaagtgcgctgccgacgaggccaaggaggtgagggaggccgaggaactgagggccgccctgccacccctggctgccgacgtggaggaacccaccctggaagccgacgtggacctgatgctgcaggaggccggcgccggaagcgtggagacacccaggggcctgatcaaggtgaccagctacgacggcgaggacaagatcggcagctacgccgtgctgagcccacaggccgtgctgaagtccgagaagctgagctgcatccacccactggccgagcaggtgatcgtgatcacccacagcggcaggaagggcaggtacgccgtggagccctaccacggcaaggtggtcgtgcccgagggccacgccatccccgtgcaggacttccaggccctgagcgagagcgccaccatcgtgtacaacgagagggagttcgtgaacaggtacctgcaccatatcgccacccacggcggagccctgaacaccgacgaggaatactacaagaccgtgaagcccagcgagcacgacggcgagtacctgtacgacatcgacaggaagcagtgcgtgaagaaagagctggtgaccggcctgggactgaccggcgagctggtggacccacccttccacgagttcgcctacgagagcctgaggaccagacccgccgctccctaccaggtgcccaccatcggcgtgtacggcgtgcccggcagcggaaagagcggcatcatcaagagcgccgtgaccaagaaagacctggtggtcagcgccaagaaagagaactgcgccgagatcatcagggacgtgaagaagatgaaaggcctggacgtgaacgcgcgcaccgtggacagcgtgctgctgaacggctgcaagcaccccgtggagaccctgtacatcgacgaggccttcgcttgccacgccggcaccctgagggccctgatcgccatcatcaggcccaagaaagccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtcgtgagcaccctgttctacgacaagaaaatgaggaccaccaaccccaaggagaccaaaatcgtgatcgacaccacaggcagcaccaagcccaagcaggacgacctgatcctgacctgcttcaggggctgggtgaagcagctgcagatcgactacaagggcaacgagatcatgaccgccgctgccagccagggcctgaccaggaagggcgtgtacgccgtgaggtacaaggtgaacgagaacccactgtacgctcccaccagcgagcacgtgaacgtgctgctgaccaggaccgaggacaggatcgtgtggaagaccctggccggcgacccctggatcaagaccctgaccgccaagtaccccggcaacttcaccgccaccatcgaagagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccacagagcagtggaacaccgtggactacttcgagaccgacaaggcccacagcgccgagatcgtgctgaaccagctgtgcgtgaggttcttcggcctggacctggacagcggcctgttcagcgcccccaccgtgccactgagcatcaggaacaaccactgggacaacagccccagcccaaacatgtacggcctgaacaaggaggtggtcaggcagctgagcaggcggtacccacagctgcccagggccgtggccaccggcagggtgtacgacatgaacaccggcaccctgaggaactacgaccccaggatcaacctggtgcccgtgaacaggcggctgccccacgccctggtgctgcaccacaacgagcacccacagagcgacttcagctccttcgtgagcaagctgaaaggcaggaccgtgctggtcgtgggcgagaagctgagcgtgcccggcaagatggtggactggctgagcgacaggcccgaggccaccttccgggccaggctggacctcggcatccccggcgacgtgcccaagtacgacatcatcttcgtgaacgtcaggaccccatacaagtaccaccattaccagcagtgcgaggaccacgccatcaagctgagcatgctgaccaagaaggcctgcctgcacctgaaccccggaggcacctgcgtgagcatcggctacggctacgccgacagggccagcgagagcatcattggcgccatcgccaggctgttcaagttcagcagggtgtgcaaacccaagagcagcctggaggaaaccgaggtgctgttcgtgttcatcggctacgaccggaaggccaggacccacaacccctacaagctgagcagcaccctgacaaacatctacaccggcagcaggctgcacgaggccggctgcgcccccagctaccacgtggtcaggggcgatatcgccaccgccaccgagggcgtgatcatcaacgctgccaacagcaagggccagcccggaggcggagtgtgcggcgccctgtacaagaagttccccgagagcttcgacctgcagcccatcgaggtgggcaaggccaggctggtgaagggcgccgctaagcacatcatccacgccgtgggccccaacttcaacaaggtgagcgaggtggaaggcgacaagcagctggccgaagcctacgagagcatcgccaagatcgtgaacgacaataactacaagagcgtggccatcccactgctcagcaccggcatcttcagcggcaacaaggacaggctgacccagagcctgaaccacctgctcaccgccctggacaccaccgatgccgacgtggccatctactgcagggacaagaagtgggagatgaccctgaaggaggccgtggccaggcgggaggccgtggaagagatctgcatcagcgacgactccagcgtgaccgagcccgacgccgagctggtgagggtgcaccccaagagctccctggccggcaggaagggctacagcaccagcgacggcaagaccttcagctacctggagggcaccaagttccaccaggccgctaaggacatcgccgagatcaacgctatgtggcccgtggccaccgaggccaacgagcaggtgtgcatgtacatcctgggcgagagcatgtccagcatcaggagcaagtgccccgtggaggaaagcgaggccagcacaccacccagcaccctgccctgcctgtgcatccacgctatgacacccgagagggtgcagcggctgaaggccagcaggcccgagcagatcaccgtgtgcagctccttcccactgcccaagtacaggatcaccggcgtgcagaagatccagtgcagccagcccatcctgttcagcccaaaggtgcccgcctacatccaccccaggaagtacctggtggagaccccacccgtggacgagacacccgagccaagcgccgagaaccagagcaccgagggcacacccgagcagccacccctgatcaccgaggacgagacaaggacccggaccccagagcccatcattatcgaggaagaggaagaggacagcatcagcctgctgagcgacggccccacccaccaggtgctgcaggtggaggccgacatccacggcccacccagcgtgtccagctccagctggagcatcccacacgccagcgacttcgacgtggacagcctgagcatcctggacaccctggagggcgccagcgtgacctccggcgccaccagcgccgagaccaacagctacttcgccaagagcatggagttcctggccaggcccgtgccagctcccaggaccgtgttcaggaacccaccccacccagctcccaggaccaggaccccaagcctggctcccagcagggcctgcagcaggaccagcctggtgagcaccccacccggcgtgaacagggtgatcaccagggaggaactggaggccctgacacccagcaggacccccagcaggtccgtgagcaggactagtctggtgtccaacccacccggcgtgaacagggtgatcaccagggaggaattcgaggccttcgtggcccagcaacagagacggttcgacgccggcgcctacatcttcagcagcgacaccggccagggacacctgcagcaaaagagcgtgaggcagaccgtgctgagcgaggtggtgctggagaggaccgagctggaaatcagctacgcccccaggctggaccaggagaaggaggaactgctcaggaagaaactgcagctgaaccccaccccagccaacaggagcaggtaccagagcaggaaggtggagaacatgaaggccatcaccgccaggcggatcctgcagggcctgggacactacctgaaggccgagggcaaggtggagtgctacaggaccctgcaccccgtgccactgtacagctccagcgtgaacagggccttctccagccccaaggtggccgtggaggcctgcaacgctatgctgaaggagaacttccccaccgtggccagctactgcatcatccccgagtacgacgcctacctggacatggtggacggcgccagctgctgcctggacaccgccagcttctgccccgccaagctgaggagcttccccaagaaacacagctacctggagcccaccatcaggagcgccgtgcccagcgccatccagaacaccctgcagaacgtgctggccgctgccaccaagaggaactgcaacgtgacccagatgagggagctgcccgtgctggacagcgctgccttcaacgtggagtgcttcaagaaatacgcctgcaacaacgagtactgggagaccttcaaggagaaccccatcaggctgaccgaagagaacgtggtgaactacatcaccaagctgaagggccccaaggccgctgccctgttcgctaagacccacaacctgaacatgctgcaggacatcccaatggacaggttcgtgatggacctgaagagggacgtgaaggtgacacccggcaccaagcacaccgaggagaggcccaaggtgcaggtgatccaggccgctgacccactggccaccgcctacctgtgcggcatccacagggagctggtgaggcggctgaacgccgtgctgctgcccaacatccacaccctgttcgacatgagcgccgaggacttcgacgccatcatcgccgagcacttccagcccggcgactgcgtgctggagaccgacatcgccagcttcgacaagagcgaggatgacgctatggccctgaccgctctgatgatcctggaggacctgggcgtggacgccgagctgctcaccctgatcgaggctgccttcggcgagatcagctccatccacctgcccaccaagaccaagttcaagttcggcgctatgatgaaaagcggaatgttcctgaccctgttcgtgaacaccgtgatcaacattgtgatcgccagcagggtgctgcgggagaggctgaccggcagcccctgcgctgccttcatcggcgacgacaacatcgtgaagggcgtgaaaagcgacaagctgatggccgacaggtgcgccacctggctgaacatggaggtgaagatcatcgacgccgtggtgggcgagaaggccccctacttctgcggcggattcatcctgtgcgacagcgtgaccggcaccgcctgcagggtggccgaccccctgaagaggctgttcaagctgggcaagccactggccgctgacgatgagcacgacgatgacaggcggagggccctgcacgaggaaagcaccaggtggaacagggtgggcatcctgagcgagctgtgcaaggccgtggagagcaggtacgagaccgtgggcaccagcatcatcgtgatggctatgaccacactggccagctccgtcaagagcttctcctacctgaggggggcccctataactctctacggctaa

SEQ ID NO: 55 - protein, 2494 aa, synthetic construct
  REGION 1..2494: note = Synthetic polypeptide
MPEKVHVDIEEDSPFLRALQRSFPQFEVEAKQVTDNDHANARAFSHLASKLIETEVDPSDTILDIGSAPARRMYSKHKYHCICPMRCAEDPDRLYKYATKLKKNCKEITDKELDKKMKELAAVMSDPDLETETMCLHDDESCRYEGQVAVYQDVYAVDGPTSLYHQANKGVRVAYWIGFDTTPFMFKNLAGAYPSYSTNWADETVLTARNIGLCSSDVMERSRRGMSILRKKYLKPSNNVLFSVGSTIYHEKRDLLRSWHLPSVFHLRGKQNYTCRCETIVSCDGYVVKRIAISPGLYGKPSGYAATMHREGFLCCKVTDTLNGERVSFPVCTYVPATLCDQMTGILATDVSADDAQKLLVGLNQRIVVNGRTQRNTNTMKNYLLPVVAQAFARWAKEYKEDQEDERPLGLRDRQLVMGCCWAFRRHKITSIYKRPDTQTIIKVNSDFHSFVLPRIGSNTLEIGLRTRIRKMLEEHKEPSPLITAEDVQEAKCAADEAKEVREAEELRAALPPLAADVEEPTLEADVDLMLQEAGAGSVETPRGLIKVTSYDGEDKIGSYAVLSPQAVLKSEKLSCIHPLAEQVIVITHSGRKGRYAVEPYHGKVVVPEGHAIPVQDFQALSESATIVYNEREFVNRYLHHIATHGGALNTDEEYYKTVKPSEHDGEYLYDIDRKQCVKKELVTGLGLTGELVDPPFHEFAYESLRTRPAAPYQVPTIGVYGVPGSGKSGIIKSAVTKKDLVVSAKKENCAEIIRDVKKMKGLDVNARTVDSVLLNGCKHPVETLYIDEAFACHAGTLRALIAIIRPKKAVLCGDPKQCGFFNMMCLKVHFNHEICTQVFHKSISRRCTKSVTSVVSTLFYDKKMRTTNPKETKIVIDTTGSTKPKQDDLILTCFRGWVKQLQIDYKGNEIMTAAASQGLTRKGVYAVRYKVNENPLYAPTSEHVNVLLTRTEDRIVWKTLAGDPWIKTLTAKYPGNFTATIEEWQAEHDAIMRHILERPDPTDVFQNKANVCWAKALVPVLKTAGIDMTTEQWNTVDYFETDKAHSAEIVLNQLCVRFFGLDLDSGLFSAPTVPLSIRNNHWDNSPSPNMYGLNKEVVRQLSRRYPQLPRAVATGRVYDMNTGTLRNYDPRINLVPVNRRLPHALVLHHNEHPQSDFSSFVSKLKGRTVLVVGEKLSVPGKMVDWLSDRPEATFRARLDLGIPGDVPKYDIIFVNVRTPYKYHHYQQCEDHAIKLSMLTKKACLHLNPGGTCVSIGYGYADRASESIIGAIARLFKFSRVCKPKSSLEETEVLFVFIGYDRKARTHNPYKLSSTLTNIYTGSRLHEAGCAPSYHVVRGDIATATEGVIINAANSKGQPGGGVCGALYKKFPESFDLQPIEVGKARLVKGAAKHIIHAVGPNFNKVSEVEGDKQLAEAYESIAKIVNDNNYKSVAIPLLSTGIFSGNKDRLTQSLNHLLTALDTTDADVAIYCRDKKWEMTLKEAVARREAVEEICISDDSSVTEPDAELVRVHPKSSLAGRKGYSTSDGKTFSYLEGTKFHQAAKDIAEINAMWPVATEANEQVCMYILGESMSSIRSKCPVEESEASTPPSTLPCLCIHAMTPERVQRLKASRPEQITVCSSFPLPKYRITGVQKIQCSQPILFSPKVPAYIHPRKYLVETPPVDETPEPSAENQSTEGTPEQPPLITEDETRTRTPEPIIIEEEEEDSISLLSDGPTHQVLQVEADIHGPPSVSSSSWSIPHASDFDVDSLSILDTLEGASVTSGATSAETNSYFAKSMEFLARPVPAPRTVFRNPPHPAPRTRTPSLAPSRACSRTSLVSTPPGVNRVITREELEALTPSRTPSRSVSRTSLVSNPPGVNRVITREEFEAFVAQQQRRFDAGAYIFSSDTGQGHLQQKSVRQTVLSEVVLERTELEISYAPRLDQEKEELLRKKLQLNPTPANRSRYQSRKVENMKAITARRILQGLGHYLKAEGKVECYRTLHPVPLYSSSVNRAFSSPKVAVEACNAMLKENFPTVASYCIIPEYDAYLDMVDGASCCLDTASFCPAKLRSFPKKHSYLEPTIRSAVPSAIQNTLQNVLAAATKRNCNVTQMRELPVLDSAAFNVECFKKYACNNEYWETFKENPIRLTEENVVNYITKLKGPKAAALFAKTHNLNMLQDIPMDRFVMDLKRDVKVTPGTKHTEERPKVQVIQAADPLATAYLCGIHRELVRRLNAVLLPNIHTLFDMSAEDFDAIIAEHFQPGDCVLETDIASFDKSEDDAMALTALMILEDLGVDAELLTLIEAAFGEISSIHLPTKTKFKFGAMMKSGMFLTLFVNTVINIVIASRVLRERLTGSPCAAFIGDDNIVKGVKSDKLMADRCATWLNMEVKIIDAVVGEKAPYFCGGFILCDSVTGTACRVADPLKRLFKLGKPLAADDEHDDDRRRALHEESTRWNRVGILSELCKAVESRYETVGTSIIVMAMTTLASSVKSFSYLRGAPITLYG

SEQ ID NO: 56 - RNA, 9739 nt, synthetic construct
  misc_feature 1..9739: note = Synthetic polynucleotide
  source 1..9739: mol_type = other RNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagaccgtgctgaccgccaggaacatcggcctgtgcagcagcgacgtgatggagaggagccggagaggcatgagcatcctgaggaagaaatacctgaagcccagcaacaacgtgctgttcagcgtgggcagcaccatctaccacgagaagagggacctgctcaggagctggcacctgcccagcgtgttccacctgaggggcaagcagaactacacctgcaggtgcgagaccatcgtgagctgcgacggctacgtggtgaagaggatcgccatcagccccggcctgtacggcaagcccagcggctacgccgctacaatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctcgtgggcctgaaccagaggatcgtggtcaacggcaggacccagaggaacaccaacacaatgaagaactacctgctgcccgtggtggcccaggctttcgccaggtgggccaaggagtacaaggaggaccaggaagacgagaggcccctgggcctgagggacaggcagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggaacacaaggagcccagcccactgatcaccgccgaggacgtgcaggaggccaagtgcgctgccgacgaggccaaggaggtgagggaggccgaggaactgagggccgccctgccacccctggctgccgacgtggaggaacccaccctggaagccgacgtggacctgatgctgcaggaggccggcgccggaagcgtggagacacccaggggcctgatcaaggtgaccagctacgacggcgaggacaagatcggcagctacgccgtgctgagcccacaggccgtgctgaagtccgagaagctgagctgcatccacccactggccgagcaggtgatcgtgatcacccacagcggcaggaagggcaggtacgccgtggagccctaccacggcaaggtggtcgtgcccgagggccacgccatccccgtgcaggacttccaggccctgagcgagagcgccaccatcgtgtacaacgagagggagttcgtgaacaggtacctgcaccatatcgccacccacggcggagccctgaacaccgacgaggaatactacaagaccgtgaagcccagcgagcacgacggcgagtacctgtacgacatcgacaggaagcagtgcgtgaagaaagagctggtgaccggcctgggactgaccggcgagctggtggacccacccttccacgagttcgcctacgagagcctgaggaccagacccgccgctccctaccaggtgcccaccatcggcgtgtacggcgtgcccggcagcggaaagagcggcatcatcaagagcgccgtgaccaagaaagacctggtggtcagcgccaagaaagagaactgcgccgagatcatcagggacgtgaagaagatgaaaggcctggacgtgaacgcgcgcaccgtggacagcgtgctgctgaacggctgcaagcaccccgtggagaccctgtacatcgacgaggccttcgcttgccacgccggcaccctgagggccctgatcgccatcatcaggcccaagaaagccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtcgtgagcaccctgttctacgacaagaaaatgaggaccaccaaccccaaggagaccaaaatcgtgatcgacaccacaggcagcaccaagcccaagcaggacgacctgatcctgacctgcttcaggggctgggtgaagcagctgcagatcgactacaagggcaacgagatcatgaccgccgctgccagccagggcctgaccaggaagggcgtgtacgccgtgaggtacaaggtgaacgagaacccactgtacgctcccaccagcgagcacgtgaacgtgctgctgaccaggaccgaggacaggatcgtgtggaagaccctggccggcgacccctggatcaagaccctgaccgccaagtaccccggcaacttcaccgccaccatcgaagagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccacagagcagtggaacaccgtggactacttcgagaccgacaaggcccacagcgccgagatcgtgctgaaccagctgtgcgtgaggttcttcggcctggacctggacagcggcctgttcagcgcccccaccgtgccactgagcatcaggaacaaccactgggacaacagccccagcccaaacatgtacggcctgaacaaggaggtggtcaggcagctgagcaggcggtacccacagctgcccagggccgtggccaccggcagggtgtacgacatgaacaccggcaccctgaggaactacgaccccaggatcaacctggtgcccgtgaacaggcggctgccccacgccctggtgctgcaccacaacgagcacccacagagcgacttcagctccttcgtgagcaagctgaaaggcaggaccgtgctggtcgtgggcgagaagctgagcgtgcccggcaagatggtggactggctgagcgacaggcccgaggccaccttccgggccaggctggacctcggcatccccggcgacgtgcccaagtacgacatcatcttcgtgaacgtcaggaccccatacaagtaccaccattaccagcagtgcgaggaccacgccatcaagctgagcatgctgaccaagaaggcctgcctgcacctgaaccccggaggcacctgcgtgagcatcggctacggctacgccgacagggccagcgagagcatcattggcgccatcgccaggctgttcaagttcagcagggtgtgcaaacccaagagcagcctggaggaaaccgaggtgctgttcgtgttcatcggctacgaccggaaggccaggacccacaacccctacaagctgagcagcaccctgacaaacatctacaccggcagcaggctgcacgaggccggctgcgcccccagctaccacgtggtcaggggcgatatcgccaccgccaccgagggcgtgatcatcaacgctgccaacagcaagggccagcccggaggcggagtgtgcggcgccctgtacaagaagttccccgagagcttcgacctgcagcccatcgaggtgggcaaggccaggctggtgaagggcgccgctaagcacatcatccacgccgtgggccccaacttcaacaaggtgagcgaggtggaaggcgacaagcagctggccgaagcctacgagagcatcgccaagatcgtgaacgacaataactacaagagcgtggccatcccactgctcagcaccggcatcttcagcggcaacaaggacaggctgacccagagcctgaaccacctgctcaccgccctggacaccaccgatgccgacgtggccatctactgcagggacaagaagtgggagatgaccctgaaggaggccgtggccaggcgggaggccgtggaagagatctgcatcagcgacgactccagcgtgaccgagcccgacgccgagctggtgagggtgcaccccaagagctccctggccggcaggaagggctacagcaccagcgacggcaagaccttcagctacctggagggcaccaagttccaccaggccgctaaggacatcgccgagatcaacgctatgtggcccgtggccaccgaggccaacgagcaggtgtgcatgtacatcctgggcgagagcatgtccagcatcaggagcaagtgccccgtggaggaaagcgaggccagcacaccacccagcaccctgccctgcctgtgcatccacgctatgacacccgagagggtgcagcggctgaaggccagcaggcccgagcagatcaccgtgtgcagctccttcccactgcccaagtacaggatcaccggcgtgcagaagatccagtgcagccagcccatcctgttcagcccaaaggtgcccgcctacatccaccccaggaagtacctggtggagaccccacccgtggacgagacacccgagccaagcgccgagaaccagagcaccgagggcacacccgagcagccacccctgatcaccgaggacgagacaaggacccggaccccagagcccatcattatcgaggaagaggaagaggacagcatcagcctgctgagcgacggccccacccaccaggtgctgcaggtggaggccgacatccacggcccacccagcgtgtccagctccagctggagcatcccacacgccagcgacttcgacgtggacagcctgagcatcctggacaccctggagggcgccagcgtgacctccggcgccaccagcgccgagaccaacagctacttcgccaagagcatggagttcctggccaggcccgtgccagctcccaggaccgtgttcaggaacccaccccacccagctcccaggaccaggaccccaagcctggctcccagcagggcctgcagcaggaccagcctggtgagcaccccacccggcgtgaacagggtgatcaccagggaggaactggaggccctgacacccagcaggacccccagcaggtccgtgagcaggactagtctggtgtccaacccacccggcgtgaacagggtgatcaccagggaggaattcgaggccttcgtggcccagcaacagagacggttcgacgccggcgcctacatcttcagcagcgacaccggccagggacacctgcagcaaaagagcgtgaggcagaccgtgctgagcgaggtggtgctggagaggaccgagctggaaatcagctacgcccccaggctggaccaggagaaggaggaactgctcaggaagaaactgcagctgaaccccaccccagccaacaggagcaggtaccagagcaggaaggtggagaacatgaaggccatcaccgccaggcggatcctgcagggcctgggacactacctgaaggccgagggcaaggtggagtgctacaggaccctgcaccccgtgccactgtacagctccagcgtgaacagggccttctccagccccaaggtggccgtggaggcctgcaacgctatgctgaaggagaacttccccaccgtggccagctactgcatcatccccgagtacgacgcctacctggacatggtggacggcgccagctgctgcctggacaccgccagcttctgccccgccaagctgaggagcttccccaagaaacacagctacctggagcccaccatcaggagcgccgtgcccagcgccatccagaacaccctgcagaacgtgctggccgctgccaccaagaggaactgcaacgtgacccagatgagggagctgcccgtgctggacagcgctgccttcaacgtggagtgcttcaagaaatacgcctgcaacaacgagtactgggagaccttcaaggagaaccccatcaggctgaccgaagagaacgtggtgaactacatcaccaagctgaagggccccaaggccgctgccctgttcgctaagacccacaacctgaacatgctgcaggacatcccaatggacaggttcgtgatggacctgaagagggacgtgaaggtgacacccggcaccaagcacaccgaggagaggcccaaggtgcaggtgatccaggccgctgacccactggccaccgcctacctgtgcggcatccacagggagctggtgaggcggctgaacgccgtgctgctgcccaacatccacaccctgttcgacatgagcgccgaggacttcgacgccatcatcgccgagcacttccagcccggcgactgcgtgctggagaccgacatcgccagcttcgacaagagcgaggatgacgctatggccctgaccgctctgatgatcctggaggacctgggcgtggacgccgagctgctcaccctgatcgaggctgccttcggcgagatcagctccatccacctgcccaccaagaccaagttcaagttcggcgctatgatgaaaagcggaatgttcctgaccctgttcgtgaacaccgtgatcaacattgtgatcgccagcagggtgctgcgggagaggctgaccggcagcccctgcgctgccttcatcggcgacgacaacatcgtgaagggcgtgaaaagcgacaagctgatggccgacaggtgcgccacctggctgaacatggaggtgaagatcatcgacgccgtggtgggcgagaaggccccctacttctgcggcggattcatcctgtgcgacagcgtgaccggcaccgcctgcagggtggccgaccccctgaagaggctgttcaagctgggcaagccactggccgctgacgatgagcacgacgatgacaggcggagggccctgcacgaggaaagcaccaggtggaacagggtgggcatcctgagcgagctgtgcaaggccgtggagagcaggtacgagaccgtgggcaccagcatcatcgtgatggctatgaccacactggccagctccgtcaagagcttctcctacctgaggggggcccctataactctctacggctaacctgaatggactacgacatagtctagtccgccaaggccgccaccatgaaggctatcctggtggtgctgctctacacctttgccacagccaatgctgacaccctgtgtattggctaccatgccaacaacagcacagacacagtggacacagtgttggagaagaatgtgacagtgacccactctgtgaacctgttggaggacaaacacaatggcaaactgtgtaaactgaggggagtggctccactgcacctgggcaagtgtaacattgctggctggattctgggcaaccctgagtgtgagtccctgagcacagcctcctcctggtcctacattgtggagacaccatcctctgacaatggcacttgttaccctggagacttcattgactatgaggaactgagggaacaactttcctctgtgtcctcctttgagaggtttgagatttttccaaagacctcctcctggccaaaccatgacagcaacaagggagtgacagcagcctgtccacatgctggagccaagtccttctacaagaacctgatttggctggtgaagaagggcaactcctacccaaaactgagcaagtcctacatcaatgacaagggcaaggaggtgctggtgctgtggggcatccaccacccaagcacctctgctgaccaacagtccctctaccagaatgctgacgcctatgtgtttgtgggctccagcagatacagcaagaagttcaagcctgagattgccatcagaccaaaggtgagggatcaggagggcaggatgaactactactggaccctggtggaacctggagacaagattacctttgaggctacaggcaacctggtggtgccaagatatgcctttgctatggagaggaatgctggctctggcatcatcatctctgacacacctgtccatgactgtaacaccacttgtcagacaccaaagggagccatcaacacctccctgccattccagaacatccacccaatcaccattggcaagtgtccaaaatatgtcaagagcaccaaactgagactggctacaggactgaggaacatcccaagcatccagagcaggggactgtttggagccattgctggcttcattgagggaggctggacagggatggtggatggctggtatggctaccaccaccagaatgaacagggctctggctatgctgctgacctgaaaagcacccagaatgccattgatgagattaccaacaaggtgaactctgtgattgagaagatgaacacccagttcacagcagtgggcaaggagttcaaccacttggagaagaggattgagaacctgaacaagaaggtggatgatggcttcctggacatctggacctacaatgctgaactgctggtgctgttggagaatgagaggaccctggactaccatgacagcaatgtgaagaacctctatgagaaggtgaggagccaacttaaaaacaatgccaaggagattggcaatggctgttttgagttctaccacaagtgtgacaacacttgtatggagtctgtgaagaatggcacctatgactacccaaaatactctgaggaggctaaactgaacagggaggagattgatggagtgaaattggagagcaccaggatttaccagatcctggccatctacagcaccgtggccagcagcctggtgctggtggtgagcctgggcgccatcagcttctggatgtgcagcaacggcagcttgcagtgcaggatctgcatctaaactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 57 - RNA, 448 nt, synthetic construct
  misc_feature 1..448: note = Synthetic polynucleotide
  source 1..448: mol_type = other RNA
gataggcggcgcatgagagaagcccagaccaattacctacccaaataggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaatagtcagcatagtacatttcatctgactaatactacaacaccaccaccatgaatagaggattctttaacatgctcggccgccgccccttcccggcccccactgccatgtggaggccgcggagaaggaggcaggcggccccgggaagcggagctactaacttcagcctgctgaagcaggctggagacgtggaggagaaccctggacct

SEQ ID NO: 58-66 - skipped (9 entries)

SEQ ID NO: 67 - DNA, 30 bp, synthetic construct
  misc_feature 1..30: note = Synthetic polynucleotide
  source 1..30: mol_type = other DNA
atggactacgacatagtctagtccgccaag

SEQ ID NO: 68 - DNA, 16 bp, synthetic construct
  misc_feature 1..16: note = Synthetic polynucleotide
  source 1..16: mol_type = other DNA
atggactacgacatag

SEQ ID NO: 69 - DNA, 15 bp, synthetic construct
  misc_feature 1..15: note = Synthetic polynucleotide
  source 1..15: mol_type = other DNA
atggactacgacata

SEQ ID NO: 70-71 - skipped (2 entries)

SEQ ID NO: 72 - DNA, 7482 bp, synthetic construct; identical to SEQ ID NO: 50
  misc_feature 1..7482: note = Synthetic polynucleotide
  source 1..7482: mol_type = other DNA

SEQ ID NO: 73 - DNA, 44 bp, synthetic construct; identical to SEQ ID NO: 49
  misc_feature 1..44: note = Synthetic polynucleotide
  source 1..44: mol_type = other DNA

SEQ ID NO: 74 - DNA, 45 bp, synthetic construct
  misc_feature 1..45: note = Synthetic polynucleotide
  source 1..45: mol_type = other DNA
gatgggcggcgcatgagagaagcccagaccaattacctacccaaa

SEQ ID NO: 75 - DNA, 45 bp, synthetic construct
  misc_feature 1..45: note = Synthetic polynucleotide
  source 1..45: mol_type = other DNA
gataggcggcgcatgagagaagcccagaccaattacctacccaaa

SEQ ID NO: 76 - DNA, 468 bp, synthetic construct; identical to SEQ ID NO: 53
  misc_feature 1..468: note = Synthetic polynucleotide
  source 1..468: mol_type = other DNA

SEQ ID NO: 77 - DNA, 44 bp, synthetic construct; identical to SEQ ID NO: 52
  misc_feature 1..44: note = Synthetic polynucleotide
  source 1..44: mol_type = other DNA

SEQ ID NO: 78 - DNA, 8038 bp, synthetic construct
  misc_feature 1..8038: note = Synthetic polynucleotide
  source 1..8038: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagaccgtgctgaccgccaggaacatcggcctgtgcagcagcgacgtgatggagaggagccggagaggcatgagcatcctgaggaagaaatacctgaagcccagcaacaacgtgctgttcagcgtgggcagcaccatctaccacgagaagagggacctgctcaggagctggcacctgcccagcgtgttccacctgaggggcaagcagaactacacctgcaggtgcgagaccatcgtgagctgcgacggctacgtggtgaagaggatcgccatcagccccggcctgtacggcaagcccagcggctacgccgctacaatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctcgtgggcctgaaccagaggatcgtggtcaacggcaggacccagaggaacaccaacacaatgaagaactacctgctgcccgtggtggcccaggctttcgccaggtgggccaaggagtacaaggaggaccaggaagacgagaggcccctgggcctgagggacaggcagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggaacacaaggagcccagcccactgatcaccgccgaggacgtgcaggaggccaagtgcgctgccgacgaggccaaggaggtgagggaggccgaggaactgagggccgccctgccacccctggctgccgacgtggaggaacccaccctggaagccgacgtggacctgatgctgcaggaggccggcgccggaagcgtggagacacccaggggcctgatcaaggtgaccagctacgacggcgaggacaagatcggcagctacgccgtgctgagcccacaggccgtgctgaagtccgagaagctgagctgcatccacccactggccgagcaggtgatcgtgatcacccacagcggcaggaagggcaggtacgccgtggagccctaccacggcaaggtggtcgtgcccgagggccacgccatccccgtgcaggacttccaggccctgagcgagagcgccaccatcgtgtacaacgagagggagttcgtgaacaggtacctgcaccatatcgccacccacggcggagccctgaacaccgacgaggaatactacaagaccgtgaagcccagcgagcacgacggcgagtacctgtacgacatcgacaggaagcagtgcgtgaagaaagagctggtgaccggcctgggactgaccggcgagctggtggacccacccttccacgagttcgcctacgagagcctgaggaccagacccgccgctccctaccaggtgcccaccatcggcgtgtacggcgtgcccggcagcggaaagagcggcatcatcaagagcgccgtgaccaagaaagacctggtggtcagcgccaagaaagagaactgcgccgagatcatcagggacgtgaagaagatgaaaggcctggacgtgaacgcgcgcaccgtggacagcgtgctgctgaacggctgcaagcaccccgtggagaccctgtacatcgacgaggccttcgcttgccacgccggcaccctgagggccctgatcgccatcatcaggcccaagaaagccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtcgtgagcaccctgttctacgacaagaaaatgaggaccaccaaccccaaggagaccaaaatcgtgatcgacaccacaggcagcaccaagcccaagcaggacgacctgatcctgacctgcttcaggggctgggtgaagcagctgcagatcgactacaagggcaacgagatcatgaccgccgctgccagccagggcctgaccaggaagggcgtgtacgccgtgaggtacaaggtgaacgagaacccactgtacgctcccaccagcgagcacgtgaacgtgctgctgaccaggaccgaggacaggatcgtgtggaagaccctggccggcgacccctggatcaagaccctgaccgccaagtaccccggcaacttcaccgccaccatcgaagagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccacagagcagtggaacaccgtggactacttcgagaccgacaaggcccacagcgccgagatcgtgctgaaccagctgtgcgtgaggttcttcggcctggacctggacagcggcctgttcagcgcccccaccgtgccactgagcatcaggaacaaccactgggacaacagccccagcccaaacatgtacggcctgaacaaggaggtggtcaggcagctgagcaggcggtacccacagctgcccagggccgtggccaccggcagggtgtacgacatgaacaccggcaccctgaggaactacgaccccaggatcaacctggtgcccgtgaacaggcggctgccccacgccctggtgctgcaccacaacgagcacccacagagcgacttcagctccttcgtgagcaagctgaaaggcaggaccgtgctggtcgtgggcgagaagctgagcgtgcccggcaagatggtggactggctgagcgacaggcccgaggccaccttccgggccaggctggacctcggcatccccggcgacgtgcccaagtacgacatcatcttcgtgaacgtcaggaccccatacaagtaccaccattaccagcagtgcgaggaccacgccatcaagctgagcatgctgaccaagaaggcctgcctgcacctgaaccccggaggcacctgcgtgagcatcggctacggctacgccgacagggccagcgagagcatcattggcgccatcgccaggctgttcaagttcagcagggtgtgcaaacccaagagcagcctggaggaaaccgaggtgctgttcgtgttcatcggctacgaccggaaggccaggacccacaacccctacaagctgagcagcaccctgacaaacatctacaccggcagcaggctgcacgaggccggctgcgcccccagctaccacgtggtcaggggcgatatcgccaccgccaccgagggcgtgatcatcaacgctgccaacagcaagggccagcccggaggcggagtgtgcggcgccctgtacaagaagttccccgagagcttcgacctgcagcccatcgaggtgggcaaggccaggctggtgaagggcgccgctaagcacatcatccacgccgtgggccccaacttcaacaaggtgagcgaggtggaaggcgacaagcagctggccgaagcctacgagagcatcgccaagatcgtgaacgacaataactacaagagcgtggccatcccactgctcagcaccggcatcttcagcggcaacaaggacaggctgacccagagcctgaaccacctgctcaccgccctggacaccaccgatgccgacgtggccatctactgcagggacaagaagtgggagatgaccctgaaggaggccgtggccaggcgggaggccgtggaagagatctgcatcagcgacgactccagcgtgaccgagcccgacgccgagctggtgagggtgcaccccaagagctccctggccggcaggaagggctacagcaccagcgacggcaagaccttcagctacctggagggcaccaagttccaccaggccgctaaggacatcgccgagatcaacgctatgtggcccgtggccaccgaggccaacgagcaggtgtgcatgtacatcctgggcgagagcatgtccagcatcaggagcaagtgccccgtggaggaaagcgaggccagcacaccacccagcaccctgccctgcctgtgcatccacgctatgacacccgagagggtgcagcggctgaaggccagcaggcccgagcagatcaccgtgtgcagctccttcccactgcccaagtacaggatcaccggcgtgcagaagatccagtgcagccagcccatcctgttcagcccaaaggtgcccgcctacatccaccccaggaagtacctggtggagaccccacccgtggacgagacacccgagccaagcgccgagaaccagagcaccgagggcacacccgagcagccacccctgatcaccgaggacgagacaaggacccggaccccagagcccatcattatcgaggaagaggaagaggacagcatcagcctgctgagcgacggccccacccaccaggtgctgcaggtggaggccgacatccacggcccacccagcgtgtccagctccagctggagcatcccacacgccagcgacttcgacgtggacagcctgagcatcctggacaccctggagggcgccagcgtgacctccggcgccaccagcgccgagaccaacagctacttcgccaagagcatggagttcctggccaggcccgtgccagctcccaggaccgtgttcaggaacccaccccacccagctcccaggaccaggaccccaagcctggctcccagcagggcctgcagcaggaccagcctggtgagcaccccacccggcgtgaacagggtgatcaccagggaggaactggaggccctgacacccagcaggacccccagcaggtccgtgagcaggactagtctggtgtccaacccacccggcgtgaacagggtgatcaccagggaggaattcgaggccttcgtggcccagcaacagagacggttcgacgccggcgcctacatcttcagcagcgacaccggccagggacacctgcagcaaaagagcgtgaggcagaccgtgctgagcgaggtggtgctggagaggaccgagctggaaatcagctacgcccccaggctggaccaggagaaggaggaactgctcaggaagaaactgcagctgaaccccaccccagccaacaggagcaggtaccagagcaggaaggtggagaacatgaaggccatcaccgccaggcggatcctgcagggcctgggacactacctgaaggccgagggcaaggtggagtgctacaggaccctgcaccccgtgccactgtacagctccagcgtgaacagggccttctccagccccaaggtggccgtggaggcctgcaacgctatgctgaaggagaacttccccaccgtggccagctactgcatcatccccgagtacgacgcctacctggacatggtggacggcgccagctgctgcctggacaccgccagcttctgccccgccaagctgaggagcttccccaagaaacacagctacctggagcccaccatcaggagcgccgtgcccagcgccatccagaacaccctgcagaacgtgctggccgctgccaccaagaggaactgcaacgtgacccagatgagggagctgcccgtgctggacagcgctgccttcaacgtggagtgcttcaagaaatacgcctgcaacaacgagtactgggagaccttcaaggagaaccccatcaggctgaccgaagagaacgtggtgaactacatcaccaagctgaagggccccaaggccgctgccctgttcgctaagacccacaacctgaacatgctgcaggacatcccaatggacaggttcgtgatggacctgaagagggacgtgaaggtgacacccggcaccaagcacaccgaggagaggcccaaggtgcaggtgatccaggccgctgacccactggccaccgcctacctgtgcggcatccacagggagctggtgaggcggctgaacgccgtgctgctgcccaacatccacaccctgttcgacatgagcgccgaggacttcgacgccatcatcgccgagcacttccagcccggcgactgcgtgctggagaccgacatcgccagcttcgacaagagcgaggatgacgctatggccctgaccgctctgatgatcctggaggacctgggcgtggacgccgagctgctcaccctgatcgaggctgccttcggcgagatcagctccatccacctgcccaccaagaccaagttcaagttcggcgctatgatgaaaagcggaatgttcctgaccctgttcgtgaacaccgtgatcaacattgtgatcgccagcagggtgctgcgggagaggctgaccggcagcccctgcgctgccttcatcggcgacgacaacatcgtgaagggcgtgaaaagcgacaagctgatggccgacaggtgcgccacctggctgaacatggaggtgaagatcatcgacgccgtggtgggcgagaaggccccctacttctgcggcggattcatcctgtgcgacagcgtgaccggcaccgcctgcagggtggccgaccccctgaagaggctgttcaagctgggcaagccactggccgctgacgatgagcacgacgatgacaggcggagggccctgcacgaggaaagcaccaggtggaacagggtgggcatcctgagcgagctgtgcaaggccgtggagagcaggtacgagaccgtgggcaccagcatcatcgtgatggctatgaccacactggccagctccgtcaagagcttctcctacctgaggggggcccctataactctctacggctaacctgaatggactacgacatagtctagtccgccaaggccgccaccactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 79 - protein, 2493 aa, synthetic construct; identical to SEQ ID NO: 51
  REGION 1..2493: note = Synthetic polypeptide

SEQ ID NO: 80 - protein, 2494 aa, synthetic construct; identical to SEQ ID NO: 55
  REGION 1..2494: note = Synthetic polypeptide

SEQ ID NO: 81 - protein, 2492 aa, synthetic construct
  REGION 1..2492: note = Synthetic polypeptide
MEKVHVDIEEDSPFLRALQRSFPQFEVEAKQVTDNDHANARAFSHLASKLIETEVDPSDTILDIGSAPARRMYSKHKYHCICPMRCAEDPDRLYKYATKLKKNCKEITDKELDKKMKELAAVMSDPDLETETMCLHDDESCRYEGQVAVYQDVYAVDGPTSLYHQANKGVRVAYWIGFDTTPFMFKNLAGAYPSYSTNWADETVLTARNIGLCSSDVMERSRRGMSILRKKYLKPSNNVLFSVGSTIYHEKRDLLRSWHLPSVFHLRGKQNYTCRCETIVSCDGYVVKRIAISPGLYGKPSGYAATMHREGFLCCKVTDTLNGERVSFPVCTYVPATLCDQMTGILATDVSADDAQKLLVGLNQRIVVNGRTQRNTNTMKNYLLPVVAQAFARWAKEYKEDQEDERPLGLRDRQLVMGCCWAFRRHKITSIYKRPDTQTIIKVNSDFHSFVLPRIGSNTLEIGLRTRIRKMLEEHKEPSPLITAEDIQEAKCAADEAKEVREAEELRAALPPLAADFEEPTLEADVDLMLQEAGAGSVETPRGLIKVTSYAGEDKIGSYAVLSPQAVLKSEKLSCIHPLAEQVIVITHSGRKGRYAVEPYHGKVVVPEGHAIPVQDFQALSESATIVYNEREFVNRYLHHIATHGGALNTDEEYYKTVKPSEHDGEYLYDIDRKQCVKKELVTGLGLTGELVDPPFHEFAYESLRTRPAAPYQVPTIGVYGVPGSGKSGIIKSAVTKKDLVVSAKKENCAEIIRDVKKMKGLDVNARTVDSVLLNGCKHPVETLYIDEAFACHAGTLRALIAIIRPKKAVLCGDPKQCGFFNMMCLKVHFNHEICTQVFHKSISRRCTKSVTSVVSTLFYDKRMRTTNPKETKIVIDTTGSTKPKQDDLILTCFRGWVKQLQIDYKGNEIMTAAASQGLTRKGVYAVRYKVNENPLYAPTSEHVNVLLTRTEDRIVWKTLAGDPWIKILTAKYPGNFTATIEEWQAEHDAIMRHILERPDPTDVFQNKANVCWAKALVPVLKTAGIDMTTEQWNTVDYFETDKAHSAEIVLNQLCVRFFGLDLDSGLFSAPTVPLSIRNNHWDNSPSPNMYGLNKEVVRQLSRRYPQLPRAVATGRVYDMNTGTLRNYDPRINLVPVNRRLPHALVLHHNEHPQSDFSSFVSKLKGRTVLVVGEKLSVPGKKVDWLSDQPEATFRARLDLGIPGDVPKYDIVFINVRTPYKYHHYQQCEDHAIKLSMLTKKACLHLNPGGTCVSIGYGYADRASESIIGAIARQFKFSRVCKPKSSHEETEVLFVFIGYDRKARTHNPYKLSSTLTNIYTGSRLHEAGCAPSYHVVRGDIATATEGVIINAANSKGQPGGGVCGALYKKFPESFDLQPIEVGKARLVKGAAKHIIHAVGPNFNKVSEVEGDKQLAEAYESIAKIVNDNNYKSVAIPLLSTGIFSGNKDRLTQSLNHLLTALDTTDADVAIYCRDKKWEMTLKEAVARREAVEEICISDDSSVTEPDAELVRVHPKSSLAGRKGYSTSDGKTFSYLEGTKFHQAAKDIAEINAMWPVATEANEQVCMYILGESMSSIRSKCPVEESEASTPPSTLPCLCIHAMTPERVQRLKASRPEQITVCSSFPLPKYRITGVQKIQCSQPILFSPKVPAYIHPRKYLVETPPVEETPESPAENQSTEGTPEQPALVNVDATRTRMPEPIIIEEEEEDSISLLSDGPTHQVLQVEADIHGSPSVSSSSWSIPHASDFDVDSLSILDTLDGASVTSGAVSAETNSYFARSMEFRARPVPAPRTVFRNPPHPAPRTRTPPLAHSRASSRTSLVSTPPGVNRVITREELEALTPSRAPSRSASRTSLVSNPPGVNRVITREEFEAFVAQQQRFDAGAYIFSSDTGQGHLQQKSVRQTVLSEVVLERTELEISYAPRLDQEKEELLRKKLQLNPTPANRSRYQSRRVENMKAITARRILQGLGHYLKAEGKVECYRTLHPVPLYSSSVNRAFSSPKVAVEACNAMLKENFPTVASYCIIPEYDAYLDMVDGASCCLDTASFCPAKLRSFPKKHSYLEPTIRSAVPSAIQNTLQNVLAAATKRNCNVTQMRELPVLDSAAFNVECFKKYACNNEYWETFKENPIRLTEENVVNYITKLKGPKAAALFAKTHNLNMLQDIPMDRFVMDLKRDVKVTPGTKHTEERPKVQVIQAADPLATADLCGIHRELVRRLNAVLLPNIHTLFDMSAEDFDAIIAEHFQPGDCVLETDIASFDKSEDDAMALTALMILEDLGVDAELLTLIEAAFGEISSIHLPTKTKFKFGAMMKSGMFLTLFVNTVINIVIASRVLRERLTGSPCAAFIGDDNIVKGVKSDKLMADRCATWLNMEVKIIDAVVGEKAPYFCGGFILCDSVTGTACRVADPLKRLFKLGKPLAVDDEHDDDRRRALHEESTRWNRVGILPELCKAVESRYETVGTSIIVMAMTTLASSVKSFSYLRGAPITLYG

SEQ ID NO: 82 - DNA, 146 bp, synthetic construct
  misc_feature 1..146: note = Synthetic polynucleotide
  source 1..146: mol_type = other DNA
aggaaacttaagtcaacacaacatatacaaaacaaacgaatctcaagcaatcaagcattctacttctattgcagcaatttaaatcatttcttttaaagcaaaagcaattttctgaaaattttcaccatttacgaacgatagccacc

SEQ ID NO: 83 - DNA, 270 bp, synthetic construct
  misc_feature 1..270: note = Synthetic polynucleotide
  source 1..270: mol_type = other DNA
actcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 84 - RNA, 1653 nt, synthetic construct
  misc_feature 1..1653: note = Synthetic polynucleotide
  source 1..1653: mol_type = other RNA
atggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgacagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagtccctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaa

SEQ ID NO: 85 - RNA, 1653 nt, synthetic construct; identical to SEQ ID NO: 84
  misc_feature 1..1653: note = Synthetic polynucleotide
  source 1..1653: mol_type = other RNA

SEQ ID NO: 86 - RNA, 1653 nt, synthetic construct; identical to SEQ ID NO: 84
  misc_feature 1..1653: note = Synthetic polynucleotide
  source 1..1653: mol_type = other RNA

SEQ ID NO: 87 - RNA, 57 nt, synthetic construct
  misc_feature 1..57: note = Synthetic polynucleotide
  source 1..57: mol_type = other RNA
atgaagttggtggttgtgggggccgggggtgttggcaaaagcgcccttacaatttga

SEQ ID NO: 88 - RNA, 66 nt, synthetic construct
  misc_feature 1..66: note = Synthetic polynucleotide
  source 1..66: mol_type = other RNA
atggatcctagacgctacgccccaatgatccgaccagcaaaactcgatgtacttccgaggaactga

SEQ ID NO: 89 - RNA, 837 nt, synthetic construct
  misc_feature 1..837: note = Synthetic polynucleotide
  source 1..837: mol_type = other RNA
atgagagtgacagcccctagaaccttactgcttctgctttggggagctgttgctctgacagagacatgggctggatctctgagcgaggtgaccggccagggcctgtgcatcggcgccgtgcccaagacccaccaggtgctgtgcaacaccacccagaagaccagcgacggcagctactacctggccgctcccaccggcaccacctgggcctgcagcaccggcctgaccccttgcatcagcaccaccatcctgaacctgaccaccgactactgcgtgctggtggagctgtggcccagggtgacctaccacagccccagctacgcctaccaccagttcgagaggagggccaagtacaagagggagcccgtgagcctgaccctggccctgctgctgggcggcctgacaatgggcggcatcgccgccggcgtgggcaccggcaccaccgccctggtggccacccagcagttccagcagctgcaggccgccatgcacgacgacctgaaggaggtggagaagtccatcaccaacctggagaagtccctgaccagcctgagcgaggtggtgctgcagaacaggaggggcctggacctgctgttcctgaaggagggcggcctgtgcgccgccctgaaggaggagtgctgcctgtacgccgaccacaccggcctggtgatcgtgggcattgtcgctggcctggccgtcctcgccgtggtggtgattggagctgtggtcgcagctgttatgtgcagaagaaagtcatccggcggaaagggaggctcctactctcaggctgcttctgctacagtgcctagagctcttatgtgtttatctcagctgtaa

SEQ ID NO: 90 - RNA, 378 nt, synthetic construct
  misc_feature 1..378: note = Synthetic polynucleotide
  source 1..378: mol_type = other RNA
atgagagtgacagcccctagaaccttactgcttctgctttggggagctgttgctctgacagagacatgggctggatcttaccacagccccagctacgcctaccaccagttcgagagggggggaggaggctccgggggaggaggctccctgaagatcagccaggccgtgcacgccgcccacgccgagatcaacgaggccggccgggaggtgatcgtgggcattgtcgctggcctggccgtcctcgccgtggtggtgattggagctgtggtcgcagctgttatgtgcagaagaaagtcatccggcggaaagggaggctcctactctcaggctgcttctgctacagtgcctagagctcttatgtgtttatctcagctgtaa

SEQ ID NO: 91 - RNA, 876 nt, synthetic construct
  misc_feature 1..876: note = Synthetic polynucleotide
  source 1..876: mol_type = other RNA
atgagagtgacagcccctagaaccttactgcttctgctttggggagctgttgctctgacagagacatgggctggatctctgagcgaggtgaccggccagggcctgtgcatcggcgccgtgcccaagacccaccaggtgctgtgcaacaccacccagaagaccagcgacggcagctactacctggccgctcccaccggcaccacctgggcctgcagcaccggcctgaccccttgcatcagcaccaccatcctgaacctgaccaccgactactgcgtgctggtggagctgtggcccagggtgacctaccacagccccagctacgcctaccaccagttcgagaggagggccaagtacaagagggagcccgtgagcctgaccctggccctgctgctgggcggcctgacaatgggcggcatcgccgccggcgtgggcaccggcaccaccgccctggtggccacccagcagttccagcagctgcaggccgccatgcacgacgacctgaaggaggtggagaagtccatcaccaacctggagaagtccctgaccagcctgagcgaggtggtgctgcagaacaggaggggcctggacctgctgttcctgaaggagggcggcctgtgcgccgccctgaaggaggagtgctgcctgtacgccgaccacaccggcctggtgatcgtgggcattgtcgctggcctggccgtcctcgccgtggtggtgattggagctgtggtcgcagctgttatgtgcagaagaaagtcatccggcggaaagggaggctcctactctcaggctgcttctgctacagtgcctagagctcttatgtgtttatctcagctgggcggcggaggcagcgactacaaggacgacgatgacaagtaa

SEQ ID NO: 92 - RNA, 417 nt, synthetic construct
  misc_feature 1..417: note = Synthetic polynucleotide
  source 1..417: mol_type = other RNA
atgagagtgacagcccctagaaccttactgcttctgctttggggagctgttgctctgacagagacatgggctggatcttaccacagccccagctacgcctaccaccagttcgagagggggggaggaggctccgggggaggaggctccctgaagatcagccaggccgtgcacgccgcccacgccgagatcaacgaggccggccgggaggtgatcgtgggcattgtcgctggcctggccgtcctcgccgtggtggtgattggagctgtggtcgcagctgttatgtgcagaagaaagtcatccggcggaaagggaggctcctactctcaggctgcttctgctacagtgcctagagctcttatgtgtttatctcagctgggcggcggaggcagcgactacaaggacgacgatgacaagtaa

SEQ ID NO: 93 - protein, 550 aa, synthetic construct
  REGION 1..550: note = Synthetic polypeptide
MEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 94 - protein, 18 aa, synthetic construct
  REGION 1..18: note = Synthetic polypeptide
MKLVVVGAGGVGKSALTI

SEQ ID NO: 95 - protein, 21 aa, synthetic construct
  REGION 1..21: note = Synthetic polypeptide
MDPRRYAPMIRPAKLDVLPRN

SEQ ID NO: 96 - protein, 278 aa, synthetic construct
  REGION 1..278: note = Synthetic polypeptide
MRVTAPRTLLLLLWGAVALTETWAGSLSEVTGQGLCIGAVPKTHQVLCNTTQKTSDGSYYLAAPTGTTWACSTGLTPCISTTILNLTTDYCVLVELWPRVTYHSPSYAYHQFERRAKYKREPVSLTLALLLGGLTMGGIAAGVGTGTTALVATQQFQQLQAAMHDDLKEVEKSITNLEKSLTSLSEVVLQNRRGLDLLFLKEGGLCAALKEECCLYADHTGLVIVGIVAGLAVLAVVVIGAVVAAVMCRRKSSGGKGGSYSQAASATVPRALMCLSQL

SEQ ID NO: 97 - protein, 125 aa, synthetic construct
  REGION 1..125: note = Synthetic polypeptide
MRVTAPRTLLLLLWGAVALTETWAGSYHSPSYAYHQFERGGGGSGGGGSLKISQAVHAAHAEINEAGREVIVGIVAGLAVLAVVVIGAVVAAVMCRRKSSGGKGGSYSQAASATVPRALMCLSQL

SEQ ID NO: 98 - protein, 291 aa, synthetic construct
  REGION 1..291: note = Synthetic polypeptide
MRVTAPRTLLLLLWGAVALTETWAGSLSEVTGQGLCIGAVPKTHQVLCNTTQKTSDGSYYLAAPTGTTWACSTGLTPCISTTILNLTTDYCVLVELWPRVTYHSPSYAYHQFERRAKYKREPVSLTLALLLGGLTMGGIAAGVGTGTTALVATQQFQQLQAAMHDDLKEVEKSITNLEKSLTSLSEVVLQNRRGLDLLFLKEGGLCAALKEECCLYADHTGLVIVGIVAGLAVLAVVVIGAVVAAVMCRRKSSGGKGGSYSQAASATVPRALMCLSQLGGGGSDYKDDDDK

SEQ ID NO: 99 - protein, 138 aa, synthetic construct
  REGION 1..138: note = Synthetic polypeptide
MRVTAPRTLLLLLWGAVALTETWAGSYHSPSYAYHQFERGGGGSGGGGSLKISQAVHAAHAEINEAGREVIVGIVAGLAVLAVVVIGAVVAAVMCRRKSSGGKGGSYSQAASATVPRALMCLSQLGGGGSDYKDDDDK

SEQ ID NO: 100 - RNA, 9690 nt, synthetic construct
  misc_feature 1..9690: note = Synthetic polynucleotide
  source 1..9690: mol_type = other RNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagaccgtgctgaccgccaggaacatcggcctgtgcagcagcgacgtgatggagaggagccggagaggcatgagcatcctgaggaagaaatacctgaagcccagcaacaacgtgctgttcagcgtgggcagcaccatctaccacgagaagagggacctgctcaggagctggcacctgcccagcgtgttccacctgaggggcaagcagaactacacctgcaggtgcgagaccatcgtgagctgcgacggctacgtggtgaagaggatcgccatcagccccggcctgtacggcaagcccagcggctacgccgctacaatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctcgtgggcctgaaccagaggatcgtggtcaacggcaggacccagaggaacaccaacacaatgaagaactacctgctgcccgtggtggcccaggctttcgccaggtgggccaaggagtacaaggaggaccaggaagacgagaggcccctgggcctgagggacaggcagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggaacacaaggagcccagcccactgatcaccgccgaggacgtgcaggaggccaagtgcgctgccgacgaggccaaggaggtgagggaggccgaggaactgagggccgccctgccacccctggctgccgacgtggaggaacccaccctggaagccgacgtggacctgatgctgcaggaggccggcgccggaagcgtggagacacccaggggcctgatcaaggtgaccagctacgacggcgaggacaagatcggcagctacgccgtgctgagcccacaggccgtgctgaagtccgagaagctgagctgcatccacccactggccgagcaggtgatcgtgatcacccacagcggcaggaagggcaggtacgccgtggagccctaccacggcaaggtggtcgtgcccgagggccacgccatccccgtgcaggacttccaggccctgagcgagagcgccaccatcgtgtacaacgagagggagttcgtgaacaggtacctgcaccatatcgccacccacggcggagccctgaacaccgacgaggaatactacaagaccgtgaagcccagcgagcacgacggcgagtacctgtacgacatcgacaggaagcagtgcgtgaagaaagagctggtgaccggcctgggactgaccggcgagctggtggacccacccttccacgagttcgcctacgagagcctgaggaccagacccgccgctccctaccaggtgcccaccatcggcgtgtacggcgtgcccggcagcggaaagagcggcatcatcaagagcgccgtgaccaagaaagacctggtggtcagcgccaagaaagagaactgcgccgagatcatcagggacgtgaagaagatgaaaggcctggacgtgaacgcgcgcaccgtggacagcgtgctgctgaacggctgcaagcaccccgtggagaccctgtacatcgacgaggccttcgcttgccacgccggcaccctgagggccctgatcgccatcatcaggcccaagaaagccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtcgtgagcaccctgttctacgacaagaaaatgaggaccaccaaccccaaggagaccaaaatcgtgatcgacaccacaggcagcaccaagcccaagcaggacgacctgatcctgacctgcttcaggggctgggtgaagcagctgcagatcgactacaagggcaacgagatcatgaccgccgctgccagccagggcctgaccaggaagggcgtgtacgccgtgaggtacaaggtgaacgagaacccactgtacgctcccaccagcgagcacgtgaacgtgctgctgaccaggaccgaggacaggatcgtgtggaagaccctggccggcgacccctggatcaagaccctgaccgccaagtaccccggcaacttcaccgccaccatcgaagagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccacagagcagtggaacaccgtggactacttcgagaccgacaaggcccacagcgccgagatcgtgctgaaccagctgtgcgtgaggttcttcggcctggacctggacagcggcctgttcagcgcccccaccgtgccactgagcatcaggaacaaccactgggacaacagccccagcccaaacatgtacggcctgaacaaggaggtggtcaggcagctgagcaggcggtacccacagctgcccagggccgtggccaccggcagggtgtacgacatgaacaccggcaccctgaggaactacgaccccaggatcaacctggtgcccgtgaacaggcggctgccccacgccctggtgctgcaccacaacgagcacccacagagcgacttcagctccttcgtgagcaagctgaaaggcaggaccgtgctggtcgtgggcgagaagctgagcgtgcccggcaagatggtggactggctgagcgacaggcccgaggccaccttccgggccaggctggacctcggcatccccggcgacgtgcccaagtacgacatcatcttcgtgaacgtcaggaccccatacaagtaccaccattaccagcagtgcgaggaccacgccatcaagctgagcatgctgaccaagaaggcctgcctgcacctgaaccccggaggcacctgcgtgagcatcggctacggctacgccgacagggccagcgagagcatcattggcgccatcgccaggctgttcaagttcagcagggtgtgcaaacccaagagcagcctggaggaaaccgaggtgctgttcgtgttcatcggctacgaccggaaggccaggacccacaacccctacaagctgagcagcaccctgacaaacatctacaccggcagcaggctgcacgaggccggctgcgcccccagctaccacgtggtcaggggcgatatcgccaccgccaccgagggcgtgatcatcaacgctgccaacagcaagggccagcccggaggcggagtgtgcggcgccctgtacaagaagttccccgagagcttcgacctgcagcccatcgaggtgggcaaggccaggctggtgaagggcgccgctaagcacatcatccacgccgtgggccccaacttcaacaaggtgagcgaggtggaaggcgacaagcagctggccgaagcctacgagagcatcgccaagatcgtgaacgacaataactacaagagcgtggccatcccactgctcagcaccggcatcttcagcggcaacaaggacaggctgacccagagcctgaaccacctgctcaccgccctggacaccaccgatgccgacgtggccatctactgcagggacaagaagtgggagatgaccctgaaggaggccgtggccaggcgggaggccgtggaagagatctgcatcagcgacgactccagcgtgaccgagcccgacgccgagctggtgagggtgcaccccaagagctccctggccggcaggaagggctacagcaccagcgacggcaagaccttcagctacctggagggcaccaagttccaccaggccgctaaggacatcgccgagatcaacgctatgtggcccgtggccaccgaggccaacgagcaggtgtgcatgtacatcctgggcgagagcatgtccagcatcaggagcaagtgccccgtggaggaaagcgaggccagcacaccacccagcaccctgccctgcctgtgcatccacgctatgacacccgagagggtgcagcggctgaaggccagcaggcccgagcagatcaccgtgtgcagctccttcccactgcccaagtacaggatcaccggcgtgcagaagatccagtgcagccagcccatcctgttcagcccaaaggtgcccgcctacatccaccccaggaagtacctggtggagaccccacccgtggacgagacacccgagccaagcgccgagaaccagagcaccgagggcacacccgagcagccacccctgatcaccgaggacgagacaaggacccggaccccagagcccatcattatcgaggaagaggaagaggacagcatcagcctgctgagcgacggccccacccaccaggtgctgcaggtggaggccgacatccacggcccacccagcgtgtccagctccagctggagcatcccacacgccagcgacttcgacgtggacagcctgagcatcctggacaccctggagggcgccagcgtgacctccggcgccaccagcgccgagaccaacagctacttcgccaagagcatggagttcctggccaggcccgtgccagctcccaggaccgtgttcaggaacccaccccacccagctcccaggaccaggaccccaagcctggctcccagcagggcctgcagcaggaccagcctggtgagcaccccacccggcgtgaacagggtgatcaccagggaggaactggaggccctgacacccagcaggacccccagcaggtccgtgagcaggactagtctggtgtccaacccacccggcgtgaacagggtgatcaccagggaggaattcgaggccttcgtggcccagcaacagagacggttcgacgccggcgcctacatcttcagcagcgacaccggccagggacacctgcagcaaaagagcgtgaggcagaccgtgctgagcgaggtggtgctggagaggaccgagctggaaatcagctacgcccccaggctggaccaggagaaggaggaactgctcaggaagaaactgcagctgaaccccaccccagccaacaggagcaggtaccagagcaggaaggtggagaacatgaaggccatcaccgccaggcggatcctgcagggcctgggacactacctgaaggccgagggcaaggtggagtgctacaggaccctgcaccccgtgccactgtacagctccagcgtgaacagggccttctccagccccaaggtggccgtggaggcctgcaacgctatgctgaaggagaacttccccaccgtggccagctactgcatcatccccgagtacgacgcctacctggacatggtggacggcgccagctgctgcctggacaccgccagcttctgccccgccaagctgaggagcttccccaagaaacacagctacctggagcccaccatcaggagcgccgtgcccagcgccatccagaacaccctgcagaacgtgctggccgctgccaccaagaggaactgcaacgtgacccagatgagggagctgcccgtgctggacagcgctgccttcaacgtggagtgcttcaagaaatacgcctgcaacaacgagtactgggagaccttcaaggagaaccccatcaggctgaccgaagagaacgtggtgaactacatcaccaagctgaagggccccaaggccgctgccctgttcgctaagacccacaacctgaacatgctgcaggacatcccaatggacaggttcgtgatggacctgaagagggacgtgaaggtgacacccggcaccaagcacaccgaggagaggcccaaggtgcaggtgatccaggccgctgacccactggccaccgcctacctgtgcggcatccacagggagctggtgaggcggctgaacgccgtgctgctgcccaacatccacaccctgttcgacatgagcgccgaggacttcgacgccatcatcgccgagcacttccagcccggcgactgcgtgctggagaccgacatcgccagcttcgacaagagcgaggatgacgctatggccctgaccgctctgatgatcctggaggacctgggcgtggacgccgagctgctcaccctgatcgaggctgccttcggcgagatcagctccatccacctgcccaccaagaccaagttcaagttcggcgctatgatgaaaagcggaatgttcctgaccctgttcgtgaacaccgtgatcaacattgtgatcgccagcagggtgctgcgggagaggctgaccggcagcccctgcgctgccttcatcggcgacgacaacatcgtgaagggcgtgaaaagcgacaagctgatggccgacaggtgcgccacctggctgaacatggaggtgaagatcatcgacgccgtggtgggcgagaaggccccctacttctgcggcggattcatcctgtgcgacagcgtgaccggcaccgcctgcagggtggccgaccccctgaagaggctgttcaagctgggcaagccactggccgctgacgatgagcacgacgatgacaggcggagggccctgcacgaggaaagcaccaggtggaacagggtgggcatcctgagcgagctgtgcaaggccgtggagagcaggtacgagaccgtgggcaccagcatcatcgtgatggctatgaccacactggccagctccgtcaagagcttctcctacctgaggggggcccctataactctctacggctaacctgaatggactacgacatagtctagtccgccaaggccgccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgacagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagtccctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 101 - RNA, 9773 nt, synthetic construct
  misc_feature 1..9773: note = Synthetic polynucleotide
  source 1..9773: mol_type = other RNA
attgacggcgtagtacacactattgaatcaaacagccgaccaattgcactaccatcacaatggagaagccagtagtaaacgtagacgtagacccccagagtccgtttgtcgtgcaactgcaaaaaagcttcccgcaatttgaggtagtagcacagcaggtcactccaaatgaccatgctaatgccagagcattttcgcatctggccagtaaactaatcgagctggaggttcctaccacagcgacgatcttggacataggcagcgcaccggctcgtagaatgttttccgagcaccagtatcattgtgtctgccccatgcgtagtccagaagacccggaccgcatgatgaaatatgccagtaaactggcggaaaaagcgtgcaagattacaaacaagaacttgcatgagaagattaaggatctccggaccgtacttgatacgccggatgctgaaacaccatcgctctgctttcacaacgatgttacctgcaacatgcgtgccgaatattccgtcatgcaggacgtgtatatcaacgctcccggaactatctatcatcaggctatgaaaggcgtgcggaccctgtactggattggcttcgacaccacccagttcatgttctcggctatggcaggttcgtaccctgcgtacaacaccaactgggccgacgagaaagtccttgaagcgcgtaacatcggactttgcagcacaaagctgagtgaaggtaggacaggaaaattgtcgataatgaggaagaaggagttgaagcccgggtcgcgggtttatttctccgtaggatcgacactttatccagaacacagagccagcttgcagagctggcatcttccatcggtgttccacttgaatggaaagcagtcgtacacttgccgctgtgatacagtggtgagttgcgaaggctacgtagtgaagaaaatcaccatcagtcccgggatcacgggagaaaccgtgggatacgcggttacacacaatagcgagggcttcttgctatgcaaagttactgacacagtaaaaggagaacgggtatcgttccctgtgtgcacgtacatcccggccaccatatgcgatcagatgactggtataatggccacggatatatcacctgacgatgcacaaaaacttctggttgggctcaaccagcgaattgtcattaacggtaggactaacaggaacaccaacaccatgcaaaattaccttctgccgatcatagcacaagggttcagcaaatgggctaaggagcgcaaggatgatcttgataacgagaaaatgctgggtactagagaacgcaagcttacgtatggctgcttgtgggcgtttcgcactaagaaagtacattcgttttatcgcccacctggaacgcagacctgcgtaaaagtcccagcctcttttagcgcttttcccatgtcgtccgtatggacgacctctttgcccatgtcgctgaggcagaaattgaaactggcattgcaaccaaagaaggaggaaaaactgctgcaggtctcggaggaattagtcatggaggccaaggctgcttttgaggatgctcaggaggaagccagagcggagaagctccgagaagcacttccaccattagtggcagacaaaggcatcgaggcagccgcagaagttgtctgcgaagtggaggggctccaggcggacatcggagcagcattagttgaaaccccgcgcggtcacgtaaggataatacctcaagcaaatgaccgtatgatcggacagtatatcgttgtctcgccaaactctgtgctgaagaatgccaaactcgcaccagcgcacccgctagcagatcaggttaagatcataacacactccggaagatcaggaaggtacgcggtcgaaccatacgacgctaaagtactgatgccagcaggaggtgccgtaccatggccagaattcctagcactgagtgagagcgccacgttagtgtacaacgaaagagagtttgtgaaccgcaaactataccacattgccatgcatggccccgccaagaatacagaagaggagcagtacaaggttacaaaggcagagcttgcagaaacagagtacgtgtttgacgtggacaagaagcgttgcgttaagaaggaagaagcctcaggtctggtcctctcgggagaactgaccaaccctccctatcatgagctagctctggagggactgaagacccgacctgcggtcccgtacaaggtcgaaacaataggagtgataggcacaccggggtcgggcaagtcagctattatcaagtcaactgtcacggcacgagatcttgttaccagcggaaagaaagaaaattgtcgcgaaattgaggccgacgtgctaagactgaggggtatgcagattacgtcgaagacagtagattcggttatgctcaacggatgccacaaagccgtagaagtgctgtacgttgacgaagcgttcgcgtgccacgcaggagcactacttgccttgattgctatcgtcaggccccgcaagaaggtagtactatgcggagaccccatgcaatgcggattcttcaacatgatgcaactaaaggtacatttcaatcaccctgaaaaagacatatgcaccaagacattctacaagtatatctcccggcgttgcacacagccagttacagctattgtatcgacactgcattacgatggaaagatgaaaaccacgaacccgtgcaagaagaacattgaaatcgatattacaggggccacaaagccgaagccaggggatatcatcctgacatgtttccgcgggtgggttaagcaattgcaaatcgactatcccggacatgaagtaatgacagccgcggcctcacaagggctaaccagaaaaggagtgtatgccgtccggcaaaaagtcaatgaaaacccactgtacgcgatcacatcagagcatgtgaacgtgttgctcacccgcactgaggacaggctagtgtggaaaaccttgcagggcgacccatggattaagcagctcactaacatacctaaaggaaactttcaggctactatagaggactgggaagctgaacacaagggaataattgctgcaataaacagccccactccccgtgccaatccgttcagctgcaagaccaacgtttgctgggcgaaagcattggaaccgatactagccacggccggtatcgtacttaccggttgccagtggagcgaactgttcccacagtttgcggatgacaaaccacattcggccatttacgccttagacgtaatttgcattaagtttttcggcatggacttgacaagcggactgttttctaaacagagcatcccactaacgtaccatcccgccgattcagcgaggccggtagctcattgggacaacagcccaggaacccgcaagtatgggtacgatcacgccattgccgccgaactctcccgtagatttccggtgttccagctagctgggaagggcacacaacttgatttgcagacggggagaaccagagttatctctgcacagcataacctggtcccggtgaaccgcaatcttcctcacgccttagtccccgagtacaaggagaagcaacccggcccggtcgaaaaattcttgaaccagttcaaacaccactcagtacttgtggtatcagaggaaaaaattgaagctccccgtaagagaatcgaatggatcgccccgattggcatagccggtgcagataagaactacaacctggctttcgggtttccgccgcaggcacggtacgacctggtgttcatcaacattggaactaaatacagaaaccaccactttcagcagtgcgaagaccatgcggcgaccttaaaaaccctttcgcgttcggccctgaattgccttaacccaggaggcaccctcgtggtgaagtcctatggctacgccgaccgcaacagtgaggacgtagtcaccgctcttgccagaaagtttgtcagggtgtctgcagcgagaccagattgtgtctcaagcaatacagaaatgtacctgattttccgacaactagacaacagccgtacacggcaattcaccccgcaccatctgaattgcgtgatttcgtccgtgtatgagggtacaagagatggagttggagccgcgccgtcataccgcaccaaaagggagaatattgctgactgtcaagaggaagcagttgtcaacgcagccaatccgctgggtagaccaggcgaaggagtctgccgtgccatctataaacgttggccgaccagttttaccgattcagccacggagacaggcaccgcaagaatgactgtgtgcctaggaaagaaagtgatccacgcggtcggccctgatttccggaagcacccagaagcagaagccttgaaattgctacaaaacgcctaccatgcagtggcagacttagtaaatgaacataacatcaagtctgtcgccattccactgctatctacaggcatttacgcagccggaaaagaccgccttgaagtatcacttaactgcttgacaaccgcgctagacagaactgacgcggacgtaaccatctattgcctggataagaagtggaaggaaagaatcgacgcggcactccaacttaaggagtctgtaacagagctgaaggatgaagatatggagatcgacgatgagttagtatggatccatccagacagttgcttgaagggaagaaagggattcagtactacaaaaggaaaattgtattcgtacttcgaaggcaccaaattccatcaagcagcaaaagacatggcggagataaaggtcctgttccctaatgaccaggaaagtaatgaacaactgtgtgcctacatattgggtgagaccatggaagcaatccgcgaaaagtgcccggtcgaccataacccgtcgtctagcccgcccaaaacgttgccgtgcctttgcatgtatgccatgacgccagaaagggtccacagacttagaagcaataacgtcaaagaagttacagtatgctcctccaccccccttcctaagcacaaaattaagaatgttcagaaggttcagtgcacgaaagtagtcctgtttaatccgcacactcccgcattcgttcccgcccgtaagtacatagaagtgccagaacagcctaccgctcctcctgcacaggccgaggaggcccccgaagttgtagcgacaccgtcaccatctacagctgataacacctcgcttgatgtcacagacatctcactggatatggatgacagtagcgaaggctcacttttttcgagctttagcggatcggacaactctattactagtatggacagttggtcgtcaggacctagttcactagagatagtagaccgaaggcaggtggtggtggctgacgttcatgccgtccaagagcctgcccctattccaccgccaaggctaaagaagatggcccgcctggcagcggcaagaaaagagcccactccaccggcaagcaatagctctgagtccctccacctctcttttggtggggtatccatgtccctcggatcaattttcgacggagagacggcccgccaggcagcggtacaacccctggcaacaggccccacggatgtgcctatgtctttcggatcgttttccgacggagagattgatgagctgagccgcagagtaactgagtccgaacccgtcctgtttggatcatttgaaccgggcgaagtgaactcaattatatcgtcccgatcagccgtatcttttcctctacgcaagcagagacgtagacgcaggagcaggaggactgaatactgactaaccggggtaggtgggtacatattttcgacggacacaggccctgggcacttgcaaaagaagtccgttctgcagaaccagcttacagaaccgaccttggagcgcaatgtcctggaaagaattcatgccccggtgctcgacacgtcgaaagaggaacaactcaaactcaggtaccagatgatgcccaccgaagccaacaaaagtaggtaccagtctcgtaaagtagaaaatcagaaagccataaccactgagcgactactgtcaggactacgactgtataactctgccacagatcagccagaatgctataagatcacctatccgaaaccattgtactccagtagcgtaccggcgaactactccgatccacagttcgctgtagctgtctgtaacaactatctgcatgagaactatccgacagtagcatcttatcagattactgacgagtacgatgcttacttggatatggtagacgggacagtcgcctgcctggacactgcaaccttctgccccgctaagcttagaagttacccgaaaaaacatgagtatagagccccgaatatccgcagtgcggttccatcagcgatgcagaacacgctacaaaatgtgctcattgccgcaactaaaagaaattgcaacgtcacgcagatgcgtgaactgccaacactggactcagcgacattcaatgtcgaatgctttcgaaaatatgcatgtaatgacgagtattgggaggagttcgctcggaagccaattaggattaccactgagtttgtcaccgcatatgtagctagactgaaaggccctaaggccgccgcactatttgcaaagacgtataatttggtcccattgcaagaagtgcctatggatagattcgtcatggacatgaaaagagacgtgaaagttacaccaggcacgaaacacacagaagaaagaccgaaagtacaagtgatacaagccgcagaacccctggcgactgcttacttatgcgggattcaccgggaattagtgcgtaggcttacggccgtcttgcttccaaacattcacacgctttttgacatgtcggcggaggattttgatgcaatcatagcagaacacttcaagcaaggcgacccggtactggagacggatatcgcatcattcgacaaaagccaagacgacgctatggcgttaaccggtctgatgatcttggaggacctgggtgtggatcaaccactactcgacttgatcgagtgcgcctttggagaaatatcatccacccatctacctacgggtactcgttttaaattcggggcgatgatgaaatccggaatgttcctcacactttttgtcaacacagttttgaatgtcgttatcgccagcagagtactagaggagcggcttaaaacgtccagatgtgcagcgttcattggcgacgacaacatcatacatggagtagtatctgacaaagaaatggctgagaggtgcgccacctggctcaacatggaggttaagatcatcgacgcagtcatcggtgagagaccaccttacttctgcggcggatttatcttgcaagattcggttacttccacagcgtgccgcgtggcggatcccctgaaaaggctgtttaagttgggtaaaccgctcccagccgacgacgagcaagacgaagacagaagacgcgctctgctagatgaaacaaaggcgtggtttagagtaggtataacaggcactttagcagtggccgtgacgacccggtatgaggtagacaatattacacctgtcctactggcattgagaacttttgcccagagcaaaagagcattccaagccatcagaggggaaataaagcatctctacggtggtcctaaatagtcagcatagtacatttcatctgactaatactacaacaccaccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgacagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagtccctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaaacgcgtgctagaccatggatcctagacgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 102 - RNA, 2086 nt, synthetic construct
  misc_feature 1..2086: note = Synthetic polynucleotide
  source 1..2086: mol_type = other RNA
aggaaacttaagtcaacacaacatatacaaaacaaacgaatctcaagcaatcaagcattctacttctattgcagcaatttaaatcatttcttttaaagcaaaagcaattttctgaaaattttcaccatttacgaacgatagccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgacagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagtccctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 103 - RNA, 8095 nt, synthetic construct
  misc_feature 1..8095: note = Synthetic polynucleotide
  source 1..8095: mol_type = other RNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagaccgtgctgaccgccaggaacatcggcctgtgcagcagcgacgtgatggagaggagccggagaggcatgagcatcctgaggaagaaatacctgaagcccagcaacaacgtgctgttcagcgtgggcagcaccatctaccacgagaagagggacctgctcaggagctggcacctgcccagcgtgttccacctgaggggcaagcagaactacacctgcaggtgcgagaccatcgtgagctgcgacggctacgtggtgaagaggatcgccatcagccccggcctgtacggcaagcccagcggctacgccgctacaatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctcgtgggcctgaaccagaggatcgtggtcaacggcaggacccagaggaacaccaacacaatgaagaactacctgctgcccgtggtggcccaggctttcgccaggtgggccaaggagtacaaggaggaccaggaagacgagaggcccctgggcctgagggacaggcagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggaacacaaggagcccagcccactgatcaccgccgaggacgtgcaggaggccaagtgcgctgccgacgaggccaaggaggtgagggaggccgaggaactgagggccgccctgccacccctggctgccgacgtggaggaacccaccctggaagccgacgtggacctgatgctgcaggaggccggcgccggaagcgtggagacacccaggggcctgatcaaggtgaccagctacgacggcgaggacaagatcggcagctacgccgtgctgagcccacaggccgtgctgaagtccgagaagctgagctgcatccacccactggccgagcaggtgatcgtgatcacccacagcggcaggaagggcaggtacgccgtggagccctaccacggcaaggtggtcgtgcccgagggccacgccatccccgtgcaggacttccaggccctgagcgagagcgccaccatcgtgtacaacgagagggagttcgtgaacaggtacctgcaccatatcgccacccacggcggagccctgaacaccgacgaggaatactacaagaccgtgaagcccagcgagcacgacggcgagtacctgtacgacatcgacaggaagcagtgcgtgaagaaagagctggtgaccggcctgggactgaccggcgagctggtggacccacccttccacgagttcgcctacgagagcctgaggaccagacccgccgctccctaccaggtgcccaccatcggcgtgtacggcgtgcccggcagcggaaagagcggcatcatcaagagcgccgtgaccaagaaagacctggtggtcagcgccaagaaagagaactgcgccgagatcatcagggacgtgaagaagatgaaaggcctggacgtgaacgcgcgcaccgtggacagcgtgctgctgaacggctgcaagcaccccgtggagaccctgtacatcgacgaggccttcgcttgccacgccggcaccctgagggccctgatcgccatcatcaggcccaagaaagccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtcgtgagcaccctgttctacgacaagaaaatgaggaccaccaaccccaaggagaccaaaatcgtgatcgacaccacaggcagcaccaagcccaagcaggacgacctgatcctgacctgcttcaggggctgggtgaagcagctgcagatcgactacaagggcaacgagatcatgaccgccgctgccagccagggcctgaccaggaagggcgtgtacgccgtgaggtacaaggtgaacgagaacccactgtacgctcccaccagcgagcacgtgaacgtgctgctgaccaggaccgaggacaggatcgtgtggaagaccctggccggcgacccctggatcaagaccctgaccgccaagtaccccggcaacttcaccgccaccatcgaagagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccacagagcagtggaacaccgtggactacttcgagaccgacaaggcccacagcgccgagatcgtgctgaaccagctgtgcgtgaggttcttcggcctggacctggacagcggcctgttcagcgcccccaccgtgccactgagcatcaggaacaaccactgggacaacagccccagcccaaacatgtacggcctgaacaaggaggtggtcaggcagctgagcaggcggtacccacagctgcccagggccgtggccaccggcagggtgtacgacatgaacaccggcaccctgaggaactacgaccccaggatcaacctggtgcccgtgaacaggcggctgccccacgccctggtgctgcaccacaacgagcacccacagagcgacttcagctccttcgtgagcaagctgaaaggcaggaccgtgctggtcgtgggcgagaagctgagcgtgcccggcaagatggtggactggctgagcgacaggcccgaggccaccttccgggccaggctggacctcggcatccccggcgacgtgcccaagtacgacatcatcttcgtgaacgtcaggaccccatacaagtaccaccattaccagcagtgcgaggaccacgccatcaagctgagcatgctgaccaagaaggcctgcctgcacctgaaccccggaggcacctgcgtgagcatcggctacggctacgccgacagggccagcgagagcatcattggcgccatcgccaggctgttcaagttcagcagggtgtgcaaacccaagagcagcctggaggaaaccgaggtgctgttcgtgttcatcggctacgaccggaaggccaggacccacaacccctacaagctgagcagcaccctgacaaacatctacaccggcagcaggctgcacgaggccggctgcgcccccagctaccacgtggtcaggggcgatatcgccaccgccaccgagggcgtgatcatcaacgctgccaacagcaagggccagcccggaggcggagtgtgcggcgccctgtacaagaagttccccgagagcttcgacctgcagcccatcgaggtgggcaaggccaggctggtgaagggcgccgctaagcacatcatccacgccgtgggccccaacttcaacaaggtgagcgaggtggaaggcgacaagcagctggccgaagcctacgagagcatcgccaagatcgtgaacgacaataactacaagagcgtggccatcccactgctcagcaccggcatcttcagcggcaacaaggacaggctgacccagagcctgaaccacctgctcaccgccctggacaccaccgatgccgacgtggccatctactgcagggacaagaagtgggagatgaccctgaaggaggccgtggccaggcgggaggccgtggaagagatctgcatcagcgacgactccagcgtgaccgagcccgacgccgagctggtgagggtgcaccccaagagctccctggccggcaggaagggctacagcaccagcgacggcaagaccttcagctacctggagggcaccaagttccaccaggccgctaaggacatcgccgagatcaacgctatgtggcccgtggccaccgaggccaacgagcaggtgtgcatgtacatcctgggcgagagcatgtccagcatcaggagcaagtgccccgtggaggaaagcgaggccagcacaccacccagcaccctgccctgcctgtgcatccacgctatgacacccgagagggtgcagcggctgaaggccagcaggcccgagcagatcaccgtgtgcagctccttcccactgcccaagtacaggatcaccggcgtgcagaagatccagtgcagccagcccatcctgttcagcccaaaggtgcccgcctacatccaccccaggaagtacctggtggagaccccacccgtggacgagacacccgagccaagcgccgagaaccagagcaccgagggcacacccgagcagccacccctgatcaccgaggacgagacaaggacccggaccccagagcccatcattatcgaggaagaggaagaggacagcatcagcctgctgagcgacggccccacccaccaggtgctgcaggtggaggccgacatccacggcccacccagcgtgtccagctccagctggagcatcccacacgccagcgacttcgacgtggacagcctgagcatcctggacaccctggagggcgccagcgtgacctccggcgccaccagcgccgagaccaacagctacttcgccaagagcatggagttcctggccaggcccgtgccagctcccaggaccgtgttcaggaacccaccccacccagctcccaggaccaggaccccaagcctggctcccagcagggcctgcagcaggaccagcctggtgagcaccccacccggcgtgaacagggtgatcaccagggaggaactggaggccctgacacccagcaggacccccagcaggtccgtgagcaggactagtctggtgtccaacccacccggcgtgaacagggtgatcaccagggaggaattcgaggccttcgtggcccagcaacagagacggttcgacgccggcgcctacatcttcagcagcgacaccggccagggacacctgcagcaaaagagcgtgaggcagaccgtgctgagcgaggtggtgctggagaggaccgagctggaaatcagctacgcccccaggctggaccaggagaaggaggaactgctcaggaagaaactgcagctgaaccccaccccagccaacaggagcaggtaccagagcaggaaggtggagaacatgaaggccatcaccgccaggcggatcctgcagggcctgggacactacctgaaggccgagggcaaggtggagtgctacaggaccctgcaccccgtgccactgtacagctccagcgtgaacagggccttctccagccccaaggtggccgtggaggcctgcaacgctatgctgaaggagaacttccccaccgtggccagctactgcatcatccccgagtacgacgcctacctggacatggtggacggcgccagctgctgcctggacaccgccagcttctgccccgccaagctgaggagcttccccaagaaacacagctacctggagcccaccatcaggagcgccgtgcccagcgccatccagaacaccctgcagaacgtgctggccgctgccaccaagaggaactgcaacgtgacccagatgagggagctgcccgtgctggacagcgctgccttcaacgtggagtgcttcaagaaatacgcctgcaacaacgagtactgggagaccttcaaggagaaccccatcaggctgaccgaagagaacgtggtgaactacatcaccaagctgaagggccccaaggccgctgccctgttcgctaagacccacaacctgaacatgctgcaggacatcccaatggacaggttcgtgatggacctgaagagggacgtgaaggtgacacccggcaccaagcacaccgaggagaggcccaaggtgcaggtgatccaggccgctgacccactggccaccgcctacctgtgcggcatccacagggagctggtgaggcggctgaacgccgtgctgctgcccaacatccacaccctgttcgacatgagcgccgaggacttcgacgccatcatcgccgagcacttccagcccggcgactgcgtgctggagaccgacatcgccagcttcgacaagagcgaggatgacgctatggccctgaccgctctgatgatcctggaggacctgggcgtggacgccgagctgctcaccctgatcgaggctgccttcggcgagatcagctccatccacctgcccaccaagaccaagttcaagttcggcgctatgatgaaaagcggaatgttcctgaccctgttcgtgaacaccgtgatcaacattgtgatcgccagcagggtgctgcgggagaggctgaccggcagcccctgcgctgccttcatcggcgacgacaacatcgtgaagggcgtgaaaagcgacaagctgatggccgacaggtgcgccacctggctgaacatggaggtgaagatcatcgacgccgtggtgggcgagaaggccccctacttctgcggcggattcatcctgtgcgacagcgtgaccggcaccgcctgcagggtggccgaccccctgaagaggctgttcaagctgggcaagccactggccgctgacgatgagcacgacgatgacaggcggagggccctgcacgaggaaagcaccaggtggaacagggtgggcatcctgagcgagctgtgcaaggccgtggagagcaggtacgagaccgtgggcaccagcatcatcgtgatggctatgaccacactggccagctccgtcaagagcttctcctacctgaggggggcccctataactctctacggctaacctgaatggactacgacatagtctagtccgccaaggccgccacccatgaagttggtggttgtgggggccgggggtgttggcaaaagcgcccttacaatttgactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 104 - RNA, 8120 nt, synthetic construct
  misc_feature 1..8120: note = Synthetic polynucleotide
  source 1..8120: mol_type = other RNA
attgacggcgtagtacacactattgaatcaaacagccgaccaattgcactaccatcacaatggagaagccagtagtaaacgtagacgtagacccccagagtccgtttgtcgtgcaactgcaaaaaagcttcccgcaatttgaggtagtagcacagcaggtcactccaaatgaccatgctaatgccagagcattttcgcatctggccagtaaactaatcgagctggaggttcctaccacagcgacgatcttggacataggcagcgcaccggctcgtagaatgttttccgagcaccagtatcattgtgtctgccccatgcgtagtccagaagacccggaccgcatgatgaaatatgccagtaaactggcggaaaaagcgtgcaagattacaaacaagaacttgcatgagaagattaaggatctccggaccgtacttgatacgccggatgctgaaacaccatcgctctgctttcacaacgatgttacctgcaacatgcgtgccgaatattccgtcatgcaggacgtgtatatcaacgctcccggaactatctatcatcaggctatgaaaggcgtgcggaccctgtactggattggcttcgacaccacccagttcatgttctcggctatggcaggttcgtaccctgcgtacaacaccaactgggccgacgagaaagtccttgaagcgcgtaacatcggactttgcagcacaaagctgagtgaaggtaggacaggaaaattgtcgataatgaggaagaaggagttgaagcccgggtcgcgggtttatttctccgtaggatcgacactttatccagaacacagagccagcttgcagagctggcatcttccatcggtgttccacttgaatggaaagcagtcgtacacttgccgctgtgatacagtggtgagttgcgaaggctacgtagtgaagaaaatcaccatcagtcccgggatcacgggagaaaccgtgggatacgcggttacacacaatagcgagggcttcttgctatgcaaagttactgacacagtaaaaggagaacgggtatcgttccctgtgtgcacgtacatcccggccaccatatgcgatcagatgactggtataatggccacggatatatcacctgacgatgcacaaaaacttctggttgggctcaaccagcgaattgtcattaacggtaggactaacaggaacaccaacaccatgcaaaattaccttctgccgatcatagcacaagggttcagcaaatgggctaaggagcgcaaggatgatcttgataacgagaaaatgctgggtactagagaacgcaagcttacgtatggctgcttgtgggcgtttcgcactaagaaagtacattcgttttatcgcccacctggaacgcagacctgcgtaaaagtcccagcctcttttagcgcttttcccatgtcgtccgtatggacgacctctttgcccatgtcgctgaggcagaaattgaaactggcattgcaaccaaagaaggaggaaaaactgctgcaggtctcggaggaattagtcatggaggccaaggctgcttttgaggatgctcaggaggaagccagagcggagaagctccgagaagcacttccaccattagtggcagacaaaggcatcgaggcagccgcagaagttgtctgcgaagtggaggggctccaggcggacatcggagcagcattagttgaaaccccgcgcggtcacgtaaggataatacctcaagcaaatgaccgtatgatcggacagtatatcgttgtctcgccaaactctgtgctgaagaatgccaaactcgcaccagcgcacccgctagcagatcaggttaagatcataacacactccggaagatcaggaaggtacgcggtcgaaccatacgacgctaaagtactgatgccagcaggaggtgccgtaccatggccagaattcctagcactgagtgagagcgccacgttagtgtacaacgaaagagagtttgtgaaccgcaaactataccacattgccatgcatggccccgccaagaatacagaagaggagcagtacaaggttacaaaggcagagcttgcagaaacagagtacgtgtttgacgtggacaagaagcgttgcgttaagaaggaagaagcctcaggtctggtcctctcgggagaactgaccaaccctccctatcatgagctagctctggagggactgaagacccgacctgcggtcccgtacaaggtcgaaacaataggagtgataggcacaccggggtcgggcaagtcagctattatcaagtcaactgtcacggcacgagatcttgttaccagcggaaagaaagaaaattgtcgcgaaattgaggccgacgtgctaagactgaggggtatgcagattacgtcgaagacagtagattcggttatgctcaacggatgccacaaagccgtagaagtgctgtacgttgacgaagcgttcgcgtgccacgcaggagcactacttgccttgattgctatcgtcaggccccgcaagaaggtagtactatgcggagaccccatgcaatgcggattcttcaacatgatgcaactaaaggtacatttcaatcaccctgaaaaagacatatgcaccaagacattctacaagtatatctcccggcgttgcacacagccagttacagctattgtatcgacactgcattacgatggaaagatgaaaaccacgaacccgtgcaagaagaacattgaaatcgatattacaggggccacaaagccgaagccaggggatatcatcctgacatgtttccgcgggtgggttaagcaattgcaaatcgactatcccggacatgaagtaatgacagccgcggcctcacaagggctaaccagaaaaggagtgtatgccgtccggcaaaaagtcaatgaaaacccactgtacgcgatcacatcagagcatgtgaacgtgttgctcacccgcactgaggacaggctagtgtggaaaaccttgcagggcgacccatggattaagcagctcactaacatacctaaaggaaactttcaggctactatagaggactgggaagctgaacacaagggaataattgctgcaataaacagccccactccccgtgccaatccgttcagctgcaagaccaacgtttgctgggcgaaagcattggaaccgatactagccacggccggtatcgtacttaccggttgccagtggagcgaactgttcccacagtttgcggatgacaaaccacattcggccatttacgccttagacgtaatttgcattaagtttttcggcatggacttgacaagcggactgttttctaaacagagcatcccactaacgtaccatcccgccgattcagcgaggccggtagctcattgggacaacagcccaggaacccgcaagtatgggtacgatcacgccattgccgccgaactctcccgtagatttccggtgttccagctagctgggaagggcacacaacttgatttgcagacggggagaaccagagttatctctgcacagcataacctggtcccggtgaaccgcaatcttcctcacgccttagtccccgagtacaaggagaagcaacccggcccggtcgaaaaattcttgaaccagttcaaacaccactcagtacttgtggtatcagaggaaaaaattgaagctccccgtaagagaatcgaatggatcgccccgattggcatagccggtgcagataagaactacaacctggctttcgggtttccgccgcaggcacggtacgacctggtgttcatcaacattggaactaaatacagaaaccaccactttcagcagtgcgaagaccatgcggcgaccttaaaaaccctttcgcgttcggccctgaattgccttaacccaggaggcaccctcgtggtgaagtcctatggctacgccgaccgcaacagtgaggacgtagtcaccgctcttgccagaaagtttgtcagggtgtctgcagcgagaccagattgtgtctcaagcaatacagaaatgtacctgattttccgacaactagacaacagccgtacacggcaattcaccccgcaccatctgaattgcgtgatttcgtccgtgtatgagggtacaagagatggagttggagccgcgccgtcataccgcaccaaaagggagaatattgctgactgtcaagaggaagcagttgtcaacgcagccaatccgctgggtagaccaggcgaaggagtctgccgtgccatctataaacgttggccgaccagttttaccgattcagccacggagacaggcaccgcaagaatgactgtgtgcctaggaaagaaagtgatccacgcggtcggccctgatttccggaagcacccagaagcagaagccttgaaattgctacaaaacgcctaccatgcagtggcagacttagtaaatgaacataacatcaagtctgtcgccattccactgctatctacaggcatttacgcagccggaaaagaccgccttgaagtatcacttaactgcttgacaaccgcgctagacagaactgacgcggacgtaaccatctattgcctggataagaagtggaaggaaagaatcgacgcggcactccaacttaaggagtctgtaacagagctgaaggatgaagatatggagatcgacgatgagttagtatggatccatccagacagttgcttgaagggaagaaagggattcagtactacaaaaggaaaattgtattcgtacttcgaaggcaccaaattccatcaagcagcaaaagacatggcggagataaaggtcctgttccctaatgaccaggaaagtaatgaacaactgtgtgcctacatattgggtgagaccatggaagcaatccgcgaaaagtgcccggtcgaccataacccgtcgtctagcccgcccaaaacgttgccgtgcctttgcatgtatgccatgacgccagaaagggtccacagacttagaagcaataacgtcaaagaagttacagtatgctcctccaccccccttcctaagcacaaaattaagaatgttcagaaggttcagtgcacgaaagtagtcctgtttaatccgcacactcccgcattcgttcccgcccgtaagtacatagaagtgccagaacagcctaccgctcctcctgcacaggccgaggaggcccccgaagttgtagcgacaccgtcaccatctacagctgataacacctcgcttgatgtcacagacatctcactggatatggatgacagtagcgaaggctcacttttttcgagctttagcggatcggacaactctattactagtatggacagttggtcgtcaggacctagttcactagagatagtagaccgaaggcaggtggtggtggctgacgttcatgccgtccaagagcctgcccctattccaccgccaaggctaaagaagatggcccgcctggcagcggcaagaaaagagcccactccaccggcaagcaatagctctgagtccctccacctctcttttggtggggtatccatgtccctcggatcaattttcgacggagagacggcccgccaggcagcggtacaacccctggcaacaggccccacggatgtgcctatgtctttcggatcgttttccgacggagagattgatgagctgagccgcagagtaactgagtccgaacccgtcctgtttggatcatttgaaccgggcgaagtgaactcaattatatcgtcccgatcagccgtatcttttcctctacgcaagcagagacgtagacgcaggagcaggaggactgaatactgactaaccggggtaggtgggtacatattttcgacggacacaggccctgggcacttgcaaaagaagtccgttctgcagaaccagcttacagaaccgaccttggagcgcaatgtcctggaaagaattcatgccccggtgctcgacacgtcgaaagaggaacaactcaaactcaggtaccagatgatgcccaccgaagccaacaaaagtaggtaccagtctcgtaaagtagaaaatcagaaagccataaccactgagcgactactgtcaggactacgactgtataactctgccacagatcagccagaatgctataagatcacctatccgaaaccattgtactccagtagcgtaccggcgaactactccgatccacagttcgctgtagctgtctgtaacaactatctgcatgagaactatccgacagtagcatcttatcagattactgacgagtacgatgcttacttggatatggtagacgggacagtcgcctgcctggacactgcaaccttctgccccgctaagcttagaagttacccgaaaaaacatgagtatagagccccgaatatccgcagtgcggttccatcagcgatgcagaacacgctacaaaatgtgctcattgccgcaactaaaagaaattgcaacgtcacgcagatgcgtgaactgccaacactggactcagcgacattcaatgtcgaatgctttcgaaaatatgcatgtaatgacgagtattgggaggagttcgctcggaagccaattaggattaccactgagtttgtcaccgcatatgtagctagactgaaaggccctaaggccgccgcactatttgcaaagacgtataatttggtcccattgcaagaagtgcctatggatagattcgtcatggacatgaaaagagacgtgaaagttacaccaggcacgaaacacacagaagaaagaccgaaagtacaagtgatacaagccgcagaacccctggcgactgcttacttatgcgggattcaccgggaattagtgcgtaggcttacggccgtcttgcttccaaacattcacacgctttttgacatgtcggcggaggattttgatgcaatcatagcagaacacttcaagcaaggcgacccggtactggagacggatatcgcatcattcgacaaaagccaagacgacgctatggcgttaaccggtctgatgatcttggaggacctgggtgtggatcaaccactactcgacttgatcgagtgcgcctttggagaaatatcatccacccatctacctacgggtactcgttttaaattcggggcgatgatgaaatccggaatgttcctcacactttttgtcaacacagttttgaatgtcgttatcgccagcagagtactagaggagcggcttaaaacgtccagatgtgcagcgttcattggcgacgacaacatcatacatggagtagtatctgacaaagaaatggctgagaggtgcgccacctggctcaacatggaggttaagatcatcgacgcagtcatcggtgagagaccaccttacttctgcggcggatttatcttgcaagattcggttacttccacagcgtgccgcgtggcggatcccctgaaaaggctgtttaagttgggtaaaccgctcccagccgacgacgagcaagacgaagacagaagacgcgctctgctagatgaaacaaaggcgtggtttagagtaggtataacaggcactttagcagtggccgtgacgacccggtatgaggtagacaatattacacctgtcctactggcattgagaacttttgcccagagcaaaagagcattccaagccatcagaggggaaataaagcatctctacggtggtcctaaatagtcagcatagtacatttcatctgactaatactacaacaccaccaccacgcgtgctagaccatggatcctagacgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 105 - RNA, 8875 nt, synthetic construct
  misc_feature 1..8875: note = Synthetic polynucleotide
  source 1..8875: mol_type = other RNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagaccgtgctgaccgccaggaacatcggcctgtgcagcagcgacgtgatggagaggagccggagaggcatgagcatcctgaggaagaaatacctgaagcccagcaacaacgtgctgttcagcgtgggcagcaccatctaccacgagaagagggacctgctcaggagctggcacctgcccagcgtgttccacctgaggggcaagcagaactacacctgcaggtgcgagaccatcgtgagctgcgacggctacgtggtgaagaggatcgccatcagccccggcctgtacggcaagcccagcggctacgccgctacaatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctcgtgggcctgaaccagaggatcgtggtcaacggcaggacccagaggaacaccaacacaatgaagaactacctgctgcccgtggtggcccaggctttcgccaggtgggccaaggagtacaaggaggaccaggaagacgagaggcccctgggcctgagggacaggcagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggaacacaaggagcccagcccactgatcaccgccgaggacgtgcaggaggccaagtgcgctgccgacgaggccaaggaggtgagggaggccgaggaactgagggccgccctgccacccctggctgccgacgtggaggaacccaccctggaagccgacgtggacctgatgctgcaggaggccggcgccggaagcgtggagacacccaggggcctgatcaaggtgaccagctacgacggcgaggacaagatcggcagctacgccgtgctgagcccacaggccgtgctgaagtccgagaagctgagctgcatccacccactggccgagcaggtgatcgtgatcacccacagcggcaggaagggcaggtacgccgtggagccctaccacggcaaggtggtcgtgcccgagggccacgccatccccgtgcaggacttccaggccctgagcgagagcgccaccatcgtgtacaacgagagggagttcgtgaacaggtacctgcaccatatcgccacccacggcggagccctgaacaccgacgaggaatactacaagaccgtgaagcccagcgagcacgacggcgagtacctgtacgacatcgacaggaagcagtgcgtgaagaaagagctggtgaccggcctgggactgaccggcgagctggtggacccacccttccacgagttcgcctacgagagcctgaggaccagacccgccgctccctaccaggtgcccaccatcggcgtgtacggcgtgcccggcagcggaaagagcggcatcatcaagagcgccgtgaccaagaaagacctggtggtcagcgccaagaaagagaactgcgccgagatcatcagggacgtgaagaagatgaaaggcctggacgtgaacgcgcgcaccgtggacagcgtgctgctgaacggctgcaagcaccccgtggagaccctgtacatcgacgaggccttcgcttgccacgccggcaccctgagggccctgatcgccatcatcaggcccaagaaagccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtcgtgagcaccctgttctacgacaagaaaatgaggaccaccaaccccaaggagaccaaaatcgtgatcgacaccacaggcagcaccaagcccaagcaggacgacctgatcctgacctgcttcaggggctgggtgaagcagctgcagatcgactacaagggcaacgagatcatgaccgccgctgccagccagggcctgaccaggaagggcgtgtacgccgtgaggtacaaggtgaacgagaacccactgtacgctcccaccagcgagcacgtgaacgtgctgctgaccaggaccgaggacaggatcgtgtggaagaccctggccggcgacccctggatcaagaccctgaccgccaagtaccccggcaacttcaccgccaccatcgaagagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccacagagcagtggaacaccgtggactacttcgagaccgacaaggcccacagcgccgagatcgtgctgaaccagctgtgcgtgaggttcttcggcctggacctggacagcggcctgttcagcgcccccaccgtgccactgagcatcaggaacaaccactgggacaacagccccagcccaaacatgtacggcctgaacaaggaggtggtcaggcagctgagcaggcggtacccacagctgcccagggccgtggccaccggcagggtgtacgacatgaacaccggcaccctgaggaactacgaccccaggatcaacctggtgcccgtgaacaggcggctgccccacgccctggtgctgcaccacaacgagcacccacagagcgacttcagctccttcgtgagcaagctgaaaggcaggaccgtgctggtcgtgggcgagaagctgagcgtgcccggcaagatggtggactggctgagcgacaggcccgaggccaccttccgggccaggctggacctcggcatccccggcgacgtgcccaagtacgacatcatcttcgtgaacgtcaggaccccatacaagtaccaccattaccagcagtgcgaggaccacgccatcaagctgagcatgctgaccaagaaggcctgcctgcacctgaaccccggaggcacctgcgtgagcatcggctacggctacgccgacagggccagcgagagcatcattggcgccatcgccaggctgttcaagttcagcagggtgtgcaaacccaagagcagcctggaggaaaccgaggtgctgttcgtgttcatcggctacgaccggaaggccaggacccacaacccctacaagctgagcagcaccctgacaaacatctacaccggcagcaggctgcacgaggccggctgcgcccccagctaccacgtggtcaggggcgatatcgccaccgccaccgagggcgtgatcatcaacgctgccaacagcaagggccagcccggaggcggagtgtgcggcgccctgtacaagaagttccccgagagcttcgacctgcagcccatcgaggtgggcaaggccaggctggtgaagggcgccgctaagcacatcatccacgccgtgggccccaacttcaacaaggtgagcgaggtggaaggcgacaagcagctggccgaagcctacgagagcatcgccaagatcgtgaacgacaataactacaagagcgtggccatcccactgctcagcaccggcatcttcagcggcaacaaggacaggctgacccagagcctgaaccacctgctcaccgccctggacaccaccgatgccgacgtggccatctactgcagggacaagaagtgggagatgaccctgaaggaggccgtggccaggcgggaggccgtggaagagatctgcatcagcgacgactccagcgtgaccgagcccgacgccgagctggtgagggtgcaccccaagagctccctggccggcaggaagggctacagcaccagcgacggcaagaccttcagctacctggagggcaccaagttccaccaggccgctaaggacatcgccgagatcaacgctatgtggcccgtggccaccgaggccaacgagcaggtgtgcatgtacatcctgggcgagagcatgtccagcatcaggagcaagtgccccgtggaggaaagcgaggccagcacaccacccagcaccctgccctgcctgtgcatccacgctatgacacccgagagggtgcagcggctgaaggccagcaggcccgagcagatcaccgtgtgcagctccttcccactgcccaagtacaggatcaccggcgtgcagaagatccagtgcagccagcccatcctgttcagcccaaaggtgcccgcctacatccaccccaggaagtacctggtggagaccccacccgtggacgagacacccgagccaagcgccgagaaccagagcaccgagggcacacccgagcagccacccctgatcaccgaggacgagacaaggacccggaccccagagcccatcattatcgaggaagaggaagaggacagcatcagcctgctgagcgacggccccacccaccaggtgctgcaggtggaggccgacatccacggcccacccagcgtgtccagctccagctggagcatcccacacgccagcgacttcgacgtggacagcctgagcatcctggacaccctggagggcgccagcgtgacctccggcgccaccagcgccgagaccaacagctacttcgccaagagcatggagttcctggccaggcccgtgccagctcccaggaccgtgttcaggaacccaccccacccagctcccaggaccaggaccccaagcctggctcccagcagggcctgcagcaggaccagcctggtgagcaccccacccggcgtgaacagggtgatcaccagggaggaactggaggccctgacacccagcaggacccccagcaggtccgtgagcaggactagtctggtgtccaacccacccggcgtgaacagggtgatcaccagggaggaattcgaggccttcgtggcccagcaacagagacggttcgacgccggcgcctacatcttcagcagcgacaccggccagggacacctgcagcaaaagagcgtgaggcagaccgtgctgagcgaggtggtgctggagaggaccgagctggaaatcagctacgcccccaggctggaccaggagaaggaggaactgctcaggaagaaactgcagctgaaccccaccccagccaacaggagcaggtaccagagcaggaaggtggagaacatgaaggccatcaccgccaggcggatcctgcagggcctgggacactacctgaaggccgagggcaaggtggagtgctacaggaccctgcaccccgtgccactgtacagctccagcgtgaacagggccttctccagccccaaggtggccgtggaggcctgcaacgctatgctgaaggagaacttccccaccgtggccagctactgcatcatccccgagtacgacgcctacctggacatggtggacggcgccagctgctgcctggacaccgccagcttctgccccgccaagctgaggagcttccccaagaaacacagctacctggagcccaccatcaggagcgccgtgcccagcgccatccagaacaccctgcagaacgtgctggccgctgccaccaagaggaactgcaacgtgacccagatgagggagctgcccgtgctggacagcgctgccttcaacgtggagtgcttcaagaaatacgcctgcaacaacgagtactgggagaccttcaaggagaaccccatcaggctgaccgaagagaacgtggtgaactacatcaccaagctgaagggccccaaggccgctgccctgttcgctaagacccacaacctgaacatgctgcaggacatcccaatggacaggttcgtgatggacctgaagagggacgtgaaggtgacacccggcaccaagcacaccgaggagaggcccaaggtgcaggtgatccaggccgctgacccactggccaccgcctacctgtgcggcatccacagggagctggtgaggcggctgaacgccgtgctgctgcccaacatccacaccctgttcgacatgagcgccgaggacttcgacgccatcatcgccgagcacttccagcccggcgactgcgtgctggagaccgacatcgccagcttcgacaagagcgaggatgacgctatggccctgaccgctctgatgatcctggaggacctgggcgtggacgccgagctgctcaccctgatcgaggctgccttcggcgagatcagctccatccacctgcccaccaagaccaagttcaagttcggcgctatgatgaaaagcggaatgttcctgaccctgttcgtgaacaccgtgatcaacattgtgatcgccagcagggtgctgcgggagaggctgaccggcagcccctgcgctgccttcatcggcgacgacaacatcgtgaagggcgtgaaaagcgacaagctgatggccgacaggtgcgccacctggctgaacatggaggtgaagatcatcgacgccgtggtgggcgagaaggccccctacttctgcggcggattcatcctgtgcgacagcgtgaccggcaccgcctgcagggtggccgaccccctgaagaggctgttcaagctgggcaagccactggccgctgacgatgagcacgacgatgacaggcggagggccctgcacgaggaaagcaccaggtggaacagggtgggcatcctgagcgagctgtgcaaggccgtggagagcaggtacgagaccgtgggcaccagcatcatcgtgatggctatgaccacactggccagctccgtcaagagcttctcctacctgaggggggcccctataactctctacggctaacctgaatggactacgacatagtctagtccgccaaggccgccaccatgagagtgacagcccctagaaccttactgcttctgctttggggagctgttgctctgacagagacatgggctggatctctgagcgaggtgaccggccagggcctgtgcatcggcgccgtgcccaagacccaccaggtgctgtgcaacaccacccagaagaccagcgacggcagctactacctggccgctcccaccggcaccacctgggcctgcagcaccggcctgaccccttgcatcagcaccaccatcctgaacctgaccaccgactactgcgtgctggtggagctgtggcccagggtgacctaccacagccccagctacgcctaccaccagttcgagaggagggccaagtacaagagggagcccgtgagcctgaccctggccctgctgctgggcggcctgacaatgggcggcatcgccgccggcgtgggcaccggcaccaccgccctggtggccacccagcagttccagcagctgcaggccgccatgcacgacgacctgaaggaggtggagaagtccatcaccaacctggagaagtccctgaccagcctgagcgaggtggtgctgcagaacaggaggggcctggacctgctgttcctgaaggagggcggcctgtgcgccgccctgaaggaggagtgctgcctgtacgccgaccacaccggcctggtgatcgtgggcattgtcgctggcctggccgtcctcgccgtggtggtgattggagctgtggtcgcagctgttatgtgcagaagaaagtcatccggcggaaagggaggctcctactctcaggctgcttctgctacagtgcctagagctcttatgtgtttatctcagctgtaaactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 106 - RNA, 8416 nt, synthetic construct
  misc_feature 1..8416: note = Synthetic polynucleotide
  source 1..8416: mol_type = other RNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagaccgtgctgaccgccaggaacatcggcctgtgcagcagcgacgtgatggagaggagccggagaggcatgagcatcctgaggaagaaatacctgaagcccagcaacaacgtgctgttcagcgtgggcagcaccatctaccacgagaagagggacctgctcaggagctggcacctgcccagcgtgttccacctgaggggcaagcagaactacacctgcaggtgcgagaccatcgtgagctgcgacggctacgtggtgaagaggatcgccatcagccccggcctgtacggcaagcccagcggctacgccgctacaatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctcgtgggcctgaaccagaggatcgtggtcaacggcaggacccagaggaacaccaacacaatgaagaactacctgctgcccgtggtggcccaggctttcgccaggtgggccaaggagtacaaggaggaccaggaagacgagaggcccctgggcctgagggacaggcagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggaacacaaggagcccagcccactgatcaccgccgaggacgtgcaggaggccaagtgcgctgccgacgaggccaaggaggtgagggaggccgaggaactgagggccgccctgccacccctggctgccgacgtggaggaacccaccctggaagccgacgtggacctgatgctgcaggaggccggcgccggaagcgtggagacacccaggggcctgatcaaggtgaccagctacgacggcgaggacaagatcggcagctacgccgtgctgagcccacaggccgtgctgaagtccgagaagctgagctgcatccacccactggccgagcaggtgatcgtgatcacccacagcggcaggaagggcaggtacgccgtggagccctaccacggcaaggtggtcgtgcccgagggccacgccatccccgtgcaggacttccaggccctgagcgagagcgccaccatcgtgtacaacgagagggagttcgtgaacaggtacctgcaccatatcgccacccacggcggagccctgaacaccgacgaggaatactacaagaccgtgaagcccagcgagcacgacggcgagtacctgtacgacatcgacaggaagcagtgcgtgaagaaagagctggtgaccggcctgggactgaccggcgagctggtggacccacccttccacgagttcgcctacgagagcctgaggaccagacccgccgctccctaccaggtgcccaccatcggcgtgtacggcgtgcccggcagcggaaagagcggcatcatcaagagcgccgtgaccaagaaagacctggtggtcagcgccaagaaagagaactgcgccgagatcatcagggacgtgaagaagatgaaaggcctggacgtgaacgcgcgcaccgtggacagcgtgctgctgaacggctgcaagcaccccgtggagaccctgtacatcgacgaggccttcgcttgccacgccggcaccctgagggccctgatcgccatcatcaggcccaagaaagccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtcgtgagcaccctgttctacgacaagaaaatgaggaccaccaaccccaaggagaccaaaatcgtgatcgacaccacaggcagcaccaagcccaagcaggacgacctgatcctgacctgcttcaggggctgggtgaagcagctgcagatcgactacaagggcaacgagatcatgaccgccgctgccagccagggcctgaccaggaagggcgtgtacgccgtgaggtacaaggtgaacgagaacccactgtacgctcccaccagcgagcacgtgaacgtgctgctgaccaggaccgaggacaggatcgtgtggaagaccctggccggcgacccctggatcaagaccctgaccgccaagtaccccggcaacttcaccgccaccatcgaagagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccacagagcagtggaacaccgtggactacttcgagaccgacaaggcccacagcgccgagatcgtgctgaaccagctgtgcgtgaggttcttcggcctggacctggacagcggcctgttcagcgcccccaccgtgccactgagcatcaggaacaaccactgggacaacagccccagcccaaacatgtacggcctgaacaaggaggtggtcaggcagctgagcaggcggtacccacagctgcccagggccgtggccaccggcagggtgtacgacatgaacaccggcaccctgaggaactacgaccccaggatcaacctggtgcccgtgaacaggcggctgccccacgccctggtgctgcaccacaacgagcacccacagagcgacttcagctccttcgtgagcaagctgaaaggcaggaccgtgctggtcgtgggcgagaagctgagcgtgcccggcaagatggtggactggctgagcgacaggcccgaggccaccttccgggccaggctggacctcggcatccccggcgacgtgcccaagtacgacatcatcttcgtgaacgtcaggaccccatacaagtaccaccattaccagcagtgcgaggaccacgccatcaagctgagcatgctgaccaagaaggcctgcctgcacctgaaccccggaggcacctgcgtgagcatcggctacggctacgccgacagggccagcgagagcatcattggcgccatcgccaggctgttcaagttcagcagggtgtgcaaacccaagagcagcctggaggaaaccgaggtgctgttcgtgttcatcggctacgaccggaaggccaggacccacaacccctacaagctgagcagcaccctgacaaacatctacaccggcagcaggctgcacgaggccggctgcgcccccagctaccacgtggtcaggggcgatatcgccaccgccaccgagggcgtgatcatcaacgctgccaacagcaagggccagcccggaggcggagtgtgcggcgccctgtacaagaagttccccgagagcttcgacctgcagcccatcgaggtgggcaaggccaggctggtgaagggcgccgctaagcacatcatccacgccgtgggccccaacttcaacaaggtgagcgaggtggaaggcgacaagcagctggccgaagcctacgagagcatcgccaagatcgtgaacgacaataactacaagagcgtggccatcccactgctcagcaccggcatcttcagcggcaacaaggacaggctgacccagagcctgaaccacctgctcaccgccctggacaccaccgatgccgacgtggccatctactgcagggacaagaagtgggagatgaccctgaaggaggccgtggccaggcgggaggccgtggaagagatctgcatcagcgacgactccagcgtgaccgagcccgacgccgagctggtgagggtgcaccccaagagctccctggccggcaggaagggctacagcaccagcgacggcaagaccttcagctacctggagggcaccaagttccaccaggccgctaaggacatcgccgagatcaacgctatgtggcccgtggccaccgaggccaacgagcaggtgtgcatgtacatcctgggcgagagcatgtccagcatcaggagcaagtgccccgtggaggaaagcgaggccagcacaccacccagcaccctgccctgcctgtgcatccacgctatgacacccgagagggtgcagcggctgaaggccagcaggcccgagcagatcaccgtgtgcagctccttcccactgcccaagtacaggatcaccggcgtgcagaagatccagtgcagccagcccatcctgttcagcccaaaggtgcccgcctacatccaccccaggaagtacctggtggagaccccacccgtggacgagacacccgagccaagcgccgagaaccagagcaccgagggcacacccgagcagccacccctgatcaccgaggacgagacaaggacccggaccccagagcccatcattatcgaggaagaggaagaggacagcatcagcctgctgagcgacggccccacccaccaggtgctgcaggtggaggccgacatccacggcccacccagcgtgtccagctccagctggagcatcccacacgccagcgacttcgacgtggacagcctgagcatcctggacaccctggagggcgccagcgtgacctccggcgccaccagcgccgagaccaacagctacttcgccaagagcatggagttcctggccaggcccgtgccagctcccaggaccgtgttcaggaacccaccccacccagctcccaggaccaggaccccaagcctggctcccagcagggcctgcagcaggaccagcctggtgagcaccccacccggcgtgaacagggtgatcaccagggaggaactggaggccctgacacccagcaggacccccagcaggtccgtgagcaggactagtctggtgtccaacccacccggcgtgaacagggtgatcaccagggaggaattcgaggccttcgtggcccagcaacagagacggttcgacgccggcgcctacatcttcagcagcgacaccggccagggacacctgcagcaaaagagcgtgaggcagaccgtgctgagcgaggtggtgctggagaggaccgagctggaaatcagctacgcccccaggctggaccaggagaaggaggaactgctcaggaagaaactgcagctgaaccccaccccagccaacaggagcaggtaccagagcaggaaggtggagaacatgaaggccatcaccgccaggcggatcctgcagggcctgggacactacctgaaggccgagggcaaggtggagtgctacaggaccctgcaccccgtgccactgtacagctccagcgtgaacagggccttctccagccccaaggtggccgtggaggcctgcaacgctatgctgaaggagaacttccccaccgtggccagctactgcatcatccccgagtacgacgcctacctggacatggtggacggcgccagctgctgcctggacaccgccagcttctgccccgccaagctgaggagcttccccaagaaacacagctacctggagcccaccatcaggagcgccgtgcccagcgccatccagaacaccctgcagaacgtgctggccgctgccaccaagaggaactgcaacgtgacccagatgagggagctgcccgtgctggacagcgctgccttcaacgtggagtgcttcaagaaatacgcctgcaacaacgagtactgggagaccttcaaggagaaccccatcaggctgaccgaagagaacgtggtgaactacatcaccaagctgaagggccccaaggccgctgccctgttcgctaagacccacaacctgaacatgctgcaggacatcccaatggacaggttcgtgatggacctgaagagggacgtgaaggtgacacccggcaccaagcacaccgaggagaggcccaaggtgcaggtgatccaggccgctgacccactggccaccgcctacctgtgcggcatccacagggagctggtgaggcggctgaacgccgtgctgctgcccaacatccacaccctgttcgacatgagcgccgaggacttcgacgccatcatcgccgagcacttccagcccggcgactgcgtgctggagaccgacatcgccagcttcgacaagagcgaggatgacgctatggccctgaccgctctgatgatcctggaggacctgggcgtggacgccgagctgctcaccctgatcgaggctgccttcggcgagatcagctccatccacctgcccaccaagaccaagttcaagttcggcgctatgatgaaaagcggaatgttcctgaccctgttcgtgaacaccgtgatcaacattgtgatcgccagcagggtgctgcgggagaggctgaccggcagcccctgcgctgccttcatcggcgacgacaacatcgtgaagggcgtgaaaagcgacaagctgatggccgacaggtgcgccacctggctgaacatggaggtgaagatcatcgacgccgtggtgggcgagaaggccccctacttctgcggcggattcatcctgtgcgacagcgtgaccggcaccgcctgcagggtggccgaccccctgaagaggctgttcaagctgggcaagccactggccgctgacgatgagcacgacgatgacaggcggagggccctgcacgaggaaagcaccaggtggaacagggtgggcatcctgagcgagctgtgcaaggccgtggagagcaggtacgagaccgtgggcaccagcatcatcgtgatggctatgaccacactggccagctccgtcaagagcttctcctacctgaggggggcccctataactctctacggctaacctgaatggactacgacatagtctagtccgccaaggccgccaccatgagagtgacagcccctagaaccttactgcttctgctttggggagctgttgctctgacagagacatgggctggatcttaccacagccccagctacgcctaccaccagttcgagagggggggaggaggctccgggggaggaggctccctgaagatcagccaggccgtgcacgccgcccacgccgagatcaacgaggccggccgggaggtgatcgtgggcattgtcgctggcctggccgtcctcgccgtggtggtgattggagctgtggtcgcagctgttatgtgcagaagaaagtcatccggcggaaagggaggctcctactctcaggctgcttctgctacagtgcctagagctcttatgtgtttatctcagctgtaaactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 107 - RNA, 8914 nt, synthetic construct
  misc_feature 1..8914: note = Synthetic polynucleotide
  source 1..8914: mol_type = other RNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagaccgtgctgaccgccaggaacatcggcctgtgcagcagcgacgtgatggagaggagccggagaggcatgagcatcctgaggaagaaatacctgaagcccagcaacaacgtgctgttcagcgtgggcagcaccatctaccacgagaagagggacctgctcaggagctggcacctgcccagcgtgttccacctgaggggcaagcagaactacacctgcaggtgcgagaccatcgtgagctgcgacggctacgtggtgaagaggatcgccatcagccccggcctgtacggcaagcccagcggctacgccgctacaatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctcgtgggcctgaaccagaggatcgtggtcaacggcaggacccagaggaacaccaacacaatgaagaactacctgctgcccgtggtggcccaggctttcgccaggtgggccaaggagtacaaggaggaccaggaagacgagaggcccctgggcctgagggacaggcagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggaacacaaggagcccagcccactgatcaccgccgaggacgtgcaggaggccaagtgcgctgccgacgaggccaaggaggtgagggaggccgaggaactgagggccgccctgccacccctggctgccgacgtggaggaacccaccctggaagccgacgtggacctgatgctgcaggaggccggcgccggaagcgtggagacacccaggggcctgatcaaggtgaccagctacgacggcgaggacaagatcggcagctacgccgtgctgagcccacaggccgtgctgaagtccgagaagctgagctgcatccacccactggccgagcaggtgatcgtgatcacccacagcggcaggaagggcaggtacgccgtggagccctaccacggcaaggtggtcgtgcccgagggccacgccatccccgtgcaggacttccaggccctgagcgagagcgccaccatcgtgtacaacgagagggagttcgtgaacaggtacctgcaccatatcgccacccacggcggagccctgaacaccgacgaggaatactacaagaccgtgaagcccagcgagcacgacggcgagtacctgtacgacatcgacaggaagcagtgcgtgaagaaagagctggtgaccggcctgggactgaccggcgagctggtggacccacccttccacgagttcgcctacgagagcctgaggaccagacccgccgctccctaccaggtgcccaccatcggcgtgtacggcgtgcccggcagcggaaagagcggcatcatcaagagcgccgtgaccaagaaagacctggtggtcagcgccaagaaagagaactgcgccgagatcatcagggacgtgaagaagatgaaaggcctggacgtgaacgcgcgcaccgtggacagcgtgctgctgaacggctgcaagcaccccgtggagaccctgtacatcgacgaggccttcgcttgccacgccggcaccctgagggccctgatcgccatcatcaggcccaagaaagccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtcgtgagcaccctgttctacgacaagaaaatgaggaccaccaaccccaaggagaccaaaatcgtgatcgacaccacaggcagcaccaagcccaagcaggacgacctgatcctgacctgcttcaggggctgggtgaagcagctgcagatcgactacaagggcaacgagatcatgaccgccgctgccagccagggcctgaccaggaagggcgtgtacgccgtgaggtacaaggtgaacgagaacccactgtacgctcccaccagcgagcacgtgaacgtgctgctgaccaggaccgaggacaggatcgtgtggaagaccctggccggcgacccctggatcaagaccctgaccgccaagtaccccggcaacttcaccgccaccatcgaagagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccacagagcagtggaacaccgtggactacttcgagaccgacaaggcccacagcgccgagatcgtgctgaaccagctgtgcgtgaggttcttcggcctggacctggacagcggcctgttcagcgcccccaccgtgccactgagcatcaggaacaaccactgggacaacagccccagcccaaacatgtacggcctgaacaaggaggtggtcaggcagctgagcaggcggtacccacagctgcccagggccgtggccaccggcagggtgtacgacatgaacaccggcaccctgaggaactacgaccccaggatcaacctggtgcccgtgaacaggcggctgccccacgccctggtgctgcaccacaacgagcacccacagagcgacttcagctccttcgtgagcaagctgaaaggcaggaccgtgctggtcgtgggcgagaagctgagcgtgcccggcaagatggtggactggctgagcgacaggcccgaggccaccttccgggccaggctggacctcggcatccccggcgacgtgcccaagtacgacatcatcttcgtgaacgtcaggaccccatacaagtaccaccattaccagcagtgcgaggaccacgccatcaagctgagcatgctgaccaagaaggcctgcctgcacctgaaccccggaggcacctgcgtgagcatcggctacggctacgccgacagggccagcgagagcatcattggcgccatcgccaggctgttcaagttcagcagggtgtgcaaacccaagagcagcctggaggaaaccgaggtgctgttcgtgttcatcggctacgaccggaaggccaggacccacaacccctacaagctgagcagcaccctgacaaacatctacaccggcagcaggctgcacgaggccggctgcgcccccagctaccacgtggtcaggggcgatatcgccaccgccaccgagggcgtgatcatcaacgctgccaacagcaagggccagcccggaggcggagtgtgcggcgccctgtacaagaagttccccgagagcttcgacctgcagcccatcgaggtgggcaaggccaggctggtgaagggcgccgctaagcacatcatccacgccgtgggccccaacttcaacaaggtgagcgaggtggaaggcgacaagcagctggccgaagcctacgagagcatcgccaagatcgtgaacgacaataactacaagagcgtggccatcccactgctcagcaccggcatcttcagcggcaacaaggacaggctgacccagagcctgaaccacctgctcaccgccctggacaccaccgatgccgacgtggccatctactgcagggacaagaagtgggagatgaccctgaaggaggccgtggccaggcgggaggccgtggaagagatctgcatcagcgacgactccagcgtgaccgagcccgacgccgagctggtgagggtgcaccccaagagctccctggccggcaggaagggctacagcaccagcgacggcaagaccttcagctacctggagggcaccaagttccaccaggccgctaaggacatcgccgagatcaacgctatgtggcccgtggccaccgaggccaacgagcaggtgtgcatgtacatcctgggcgagagcatgtccagcatcaggagcaagtgccccgtggaggaaagcgaggccagcacaccacccagcaccctgccctgcctgtgcatccacgctatgacacccgagagggtgcagcggctgaaggccagcaggcccgagcagatcaccgtgtgcagctccttcccactgcccaagtacaggatcaccggcgtgcagaagatccagtgcagccagcccatcctgttcagcccaaaggtgcccgcctacatccaccccaggaagtacctggtggagaccccacccgtggacgagacacccgagccaagcgccgagaaccagagcaccgagggcacacccgagcagccacccctgatcaccgaggacgagacaaggacccggaccccagagcccatcattatcgaggaagaggaagaggacagcatcagcctgctgagcgacggccccacccaccaggtgctgcaggtggaggccgacatccacggcccacccagcgtgtccagctccagctggagcatcccacacgccagcgacttcgacgtggacagcctgagcatcctggacaccctggagggcgccagcgtgacctccggcgccaccagcgccgagaccaacagctacttcgccaagagcatggagttcctggccaggcccgtgccagctcccaggaccgtgttcaggaacccaccccacccagctcccaggaccaggaccccaagcctggctcccagcagggcctgcagcaggaccagcctggtgagcaccccacccggcgtgaacagggtgatcaccagggaggaactggaggccctgacacccagcaggacccccagcaggtccgtgagcaggactagtctggtgtccaacccacccggcgtgaacagggtgatcaccagggaggaattcgaggccttcgtggcccagcaacagagacggttcgacgccggcgcctacatcttcagcagcgacaccggccagggacacctgcagcaaaagagcgtgaggcagaccgtgctgagcgaggtggtgctggagaggaccgagctggaaatcagctacgcccccaggctggaccaggagaaggaggaactgctcaggaagaaactgcagctgaaccccaccccagccaacaggagcaggtaccagagcaggaaggtggagaacatgaaggccatcaccgccaggcggatcctgcagggcctgggacactacctgaaggccgagggcaaggtggagtgctacaggaccctgcaccccgtgccactgtacagctccagcgtgaacagggccttctccagccccaaggtggccgtggaggcctgcaacgctatgctgaaggagaacttccccaccgtggccagctactgcatcatccccgagtacgacgcctacctggacatggtggacggcgccagctgctgcctggacaccgccagcttctgccccgccaagctgaggagcttccccaagaaacacagctacctggagcccaccatcaggagcgccgtgcccagcgccatccagaacaccctgcagaacgtgctggccgctgccaccaagaggaactgcaacgtgacccagatgagggagctgcccgtgctggacagcgctgccttcaacgtggagtgcttcaagaaatacgcctgcaacaacgagtactgggagaccttcaaggagaaccccatcaggctgaccgaagagaacgtggtgaactacatcaccaagctgaagggccccaaggccgctgccctgttcgctaagacccacaacctgaacatgctgcaggacatcccaatggacaggttcgtgatggacctgaagagggacgtgaaggtgacacccggcaccaagcacaccgaggagaggcccaaggtgcaggtgatccaggccgctgacccactggccaccgcctacctgtgcggcatccacagggagctggtgaggcggctgaacgccgtgctgctgcccaacatccacaccctgttcgacatgagcgccgaggacttcgacgccatcatcgccgagcacttccagcccggcgactgcgtgctggagaccgacatcgccagcttcgacaagagcgaggatgacgctatggccctgaccgctctgatgatcctggaggacctgggcgtggacgccgagctgctcaccctgatcgaggctgccttcggcgagatcagctccatccacctgcccaccaagaccaagttcaagttcggcgctatgatgaaaagcggaatgttcctgaccctgttcgtgaacaccgtgatcaacattgtgatcgccagcagggtgctgcgggagaggctgaccggcagcccctgcgctgccttcatcggcgacgacaacatcgtgaagggcgtgaaaagcgacaagctgatggccgacaggtgcgccacctggctgaacatggaggtgaagatcatcgacgccgtggtgggcgagaaggccccctacttctgcggcggattcatcctgtgcgacagcgtgaccggcaccgcctgcagggtggccgaccccctgaagaggctgttcaagctgggcaagccactggccgctgacgatgagcacgacgatgacaggcggagggccctgcacgaggaaagcaccaggtggaacagggtgggcatcctgagcgagctgtgcaaggccgtggagagcaggtacgagaccgtgggcaccagcatcatcgtgatggctatgaccacactggccagctccgtcaagagcttctcctacctgaggggggcccctataactctctacggctaacctgaatggactacgacatagtctagtccgccaaggccgccaccatgagagtgacagcccctagaaccttactgcttctgctttggggagctgttgctctgacagagacatgggctggatctctgagcgaggtgaccggccagggcctgtgcatcggcgccgtgcccaagacccaccaggtgctgtgcaacaccacccagaagaccagcgacggcagctactacctggccgctcccaccggcaccacctgggcctgcagcaccggcctgaccccttgcatcagcaccaccatcctgaacctgaccaccgactactgcgtgctggtggagctgtggcccagggtgacctaccacagccccagctacgcctaccaccagttcgagaggagggccaagtacaagagggagcccgtgagcctgaccctggccctgctgctgggcggcctgacaatgggcggcatcgccgccggcgtgggcaccggcaccaccgccctggtggccacccagcagttccagcagctgcaggccgccatgcacgacgacctgaaggaggtggagaagtccatcaccaacctggagaagtccctgaccagcctgagcgaggtggtgctgcagaacaggaggggcctggacctgctgttcctgaaggagggcggcctgtgcgccgccctgaaggaggagtgctgcctgtacgccgaccacaccggcctggtgatcgtgggcattgtcgctggcctggccgtcctcgccgtggtggtgattggagctgtggtcgcagctgttatgtgcagaagaaagtcatccggcggaaagggaggctcctactctcaggctgcttctgctacagtgcctagagctcttatgtgtttatctcagctgggcggcggaggcagcgactacaaggacgacgatgacaagtaaactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 108 - RNA, 8455 nt, synthetic construct
  misc_feature 1..8455: note = Synthetic polynucleotide
  source 1..8455: mol_type = other RNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagaccgtgctgaccgccaggaacatcggcctgtgcagcagcgacgtgatggagaggagccggagaggcatgagcatcctgaggaagaaatacctgaagcccagcaacaacgtgctgttcagcgtgggcagcaccatctaccacgagaagagggacctgctcaggagctggcacctgcccagcgtgttccacctgaggggcaagcagaactacacctgcaggtgcgagaccatcgtgagctgcgacggctacgtggtgaagaggatcgccatcagccccggcctgtacggcaagcccagcggctacgccgctacaatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctcgtgggcctgaaccagaggatcgtggtcaacggcaggacccagaggaacaccaacacaatgaagaactacctgctgcccgtggtggcccaggctttcgccaggtgggccaaggagtacaaggaggaccaggaagacgagaggcccctgggcctgagggacaggcagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggaacacaaggagcccagcccactgatcaccgccgaggacgtgcaggaggccaagtgcgctgccgacgaggccaaggaggtgagggaggccgaggaactgagggccgccctgccacccctggctgccgacgtggaggaacccaccctggaagccgacgtggacctgatgctgcaggaggccggcgccggaagcgtggagacacccaggggcctgatcaaggtgaccagctacgacggcgaggacaagatcggcagctacgccgtgctgagcccacaggccgtgctgaagtccgagaagctgagctgcatccacccactggccgagcaggtgatcgtgatcacccacagcggcaggaagggcaggtacgccgtggagccctaccacggcaaggtggtcgtgcccgagggccacgccatccccgtgcaggacttccaggccctgagcgagagcgccaccatcgtgtacaacgagagggagttcgtgaacaggtacctgcaccatatcgccacccacggcggagccctgaacaccgacgaggaatactacaagaccgtgaagcccagcgagcacgacggcgagtacctgtacgacatcgacaggaagcagtgcgtgaagaaagagctggtgaccggcctgggactgaccggcgagctggtggacccacccttccacgagttcgcctacgagagcctgaggaccagacccgccgctccctaccaggtgcccaccatcggcgtgtacggcgtgcccggcagcggaaagagcggcatcatcaagagcgccgtgaccaagaaagacctggtggtcagcgccaagaaagagaactgcgccgagatcatcagggacgtgaagaagatgaaaggcctggacgtgaacgcgcgcaccgtggacagcgtgctgctgaacggctgcaagcaccccgtggagaccctgtacatcgacgaggccttcgcttgccacgccggcaccctgagggccctgatcgccatcatcaggcccaagaaagccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtcgtgagcaccctgttctacgacaagaaaatgaggaccaccaaccccaaggagaccaaaatcgtgatcgacaccacaggcagcaccaagcccaagcaggacgacctgatcctgacctgcttcaggggctgggtgaagcagctgcagatcgactacaagggcaacgagatcatgaccgccgctgccagccagggcctgaccaggaagggcgtgtacgccgtgaggtacaaggtgaacgagaacccactgtacgctcccaccagcgagcacgtgaacgtgctgctgaccaggaccgaggacaggatcgtgtggaagaccctggccggcgacccctggatcaagaccctgaccgccaagtaccccggcaacttcaccgccaccatcgaagagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccacagagcagtggaacaccgtggactacttcgagaccgacaaggcccacagcgccgagatcgtgctgaaccagctgtgcgtgaggttcttcggcctggacctggacagcggcctgttcagcgcccccaccgtgccactgagcatcaggaacaaccactgggacaacagccccagcccaaacatgtacggcctgaacaaggaggtggtcaggcagctgagcaggcggtacccacagctgcccagggccgtggccaccggcagggtgtacgacatgaacaccggcaccctgaggaactacgaccccaggatcaacctggtgcccgtgaacaggcggctgccccacgccctggtgctgcaccacaacgagcacccacagagcgacttcagctccttcgtgagcaagctgaaaggcaggaccgtgctggtcgtgggcgagaagctgagcgtgcccggcaagatggtggactggctgagcgacaggcccgaggccaccttccgggccaggctggacctcggcatccccggcgacgtgcccaagtacgacatcatcttcgtgaacgtcaggaccccatacaagtaccaccattaccagcagtgcgaggaccacgccatcaagctgagcatgctgaccaagaaggcctgcctgcacctgaaccccggaggcacctgcgtgagcatcggctacggctacgccgacagggccagcgagagcatcattggcgccatcgccaggctgttcaagttcagcagggtgtgcaaacccaagagcagcctggaggaaaccgaggtgctgttcgtgttcatcggctacgaccggaaggccaggacccacaacccctacaagctgagcagcaccctgacaaacatctacaccggcagcaggctgcacgaggccggctgcgcccccagctaccacgtggtcaggggcgatatcgccaccgccaccgagggcgtgatcatcaacgctgccaacagcaagggccagcccggaggcggagtgtgcggcgccctgtacaagaagttccccgagagcttcgacctgcagcccatcgaggtgggcaaggccaggctggtgaagggcgccgctaagcacatcatccacgccgtgggccccaacttcaacaaggtgagcgaggtggaaggcgacaagcagctggccgaagcctacgagagcatcgccaagatcgtgaacgacaataactacaagagcgtggccatcccactgctcagcaccggcatcttcagcggcaacaaggacaggctgacccagagcctgaaccacctgctcaccgccctggacaccaccgatgccgacgtggccatctactgcagggacaagaagtgggagatgaccctgaaggaggccgtggccaggcgggaggccgtggaagagatctgcatcagcgacgactccagcgtgaccgagcccgacgccgagctggtgagggtgcaccccaagagctccctggccggcaggaagggctacagcaccagcgacggcaagaccttcagctacctggagggcaccaagttccaccaggccgctaaggacatcgccgagatcaacgctatgtggcccgtggccaccgaggccaacgagcaggtgtgcatgtacatcctgggcgagagcatgtccagcatcaggagcaagtgccccgtggaggaaagcgaggccagcacaccacccagcaccctgccctgcctgtgcatccacgctatgacacccgagagggtgcagcggctgaaggccagcaggcccgagcagatcaccgtgtgcagctccttcccactgcccaagtacaggatcaccggcgtgcagaagatccagtgcagccagcccatcctgttcagcccaaaggtgcccgcctacatccaccccaggaagtacctggtggagaccccacccgtggacgagacacccgagccaagcgccgagaaccagagcaccgagggcacacccgagcagccacccctgatcaccgaggacgagacaaggacccggaccccagagcccatcattatcgaggaagaggaagaggacagcatcagcctgctgagcgacggccccacccaccaggtgctgcaggtggaggccgacatccacggcccacccagcgtgtccagctccagctggagcatcccacacgccagcgacttcgacgtggacagcctgagcatcctggacaccctggagggcgccagcgtgacctccggcgccaccagcgccgagaccaacagctacttcgccaagagcatggagttcctggccaggcccgtgccagctcccaggaccgtgttcaggaacccaccccacccagctcccaggaccaggaccccaagcctggctcccagcagggcctgcagcaggaccagcctggtgagcaccccacccggcgtgaacagggtgatcaccagggaggaactggaggccctgacacccagcaggacccccagcaggtccgtgagcaggactagtctggtgtccaacccacccggcgtgaacagggtgatcaccagggaggaattcgaggccttcgtggcccagcaacagagacggttcgacgccggcgcctacatcttcagcagcgacaccggccagggacacctgcagcaaaagagcgtgaggcagaccgtgctgagcgaggtggtgctggagaggaccgagctggaaatcagctacgcccccaggctggaccaggagaaggaggaactgctcaggaagaaactgcagctgaaccccaccccagccaacaggagcaggtaccagagcaggaaggtggagaacatgaaggccatcaccgccaggcggatcctgcagggcctgggacactacctgaaggccgagggcaaggtggagtgctacaggaccctgcaccccgtgccactgtacagctccagcgtgaacagggccttctccagccccaaggtggccgtggaggcctgcaacgctatgctgaaggagaacttccccaccgtggccagctactgcatcatccccgagtacgacgcctacctggacatggtggacggcgccagctgctgcctggacaccgccagcttctgccccgccaagctgaggagcttccccaagaaacacagctacctggagcccaccatcaggagcgccgtgcccagcgccatccagaacaccctgcagaacgtgctggccgctgccaccaagaggaactgcaacgtgacccagatgagggagctgcccgtgctggacagcgctgccttcaacgtggagtgcttcaagaaatacgcctgcaacaacgagtactgggagaccttcaaggagaaccccatcaggctgaccgaagagaacgtggtgaactacatcaccaagctgaagggccccaaggccgctgccctgttcgctaagacccacaacctgaacatgctgcaggacatcccaatggacaggttcgtgatggacctgaagagggacgtgaaggtgacacccggcaccaagcacaccgaggagaggcccaaggtgcaggtgatccaggccgctgacccactggccaccgcctacctgtgcggcatccacagggagctggtgaggcggctgaacgccgtgctgctgcccaacatccacaccctgttcgacatgagcgccgaggacttcgacgccatcatcgccgagcacttccagcccggcgactgcgtgctggagaccgacatcgccagcttcgacaagagcgaggatgacgctatggccctgaccgctctgatgatcctggaggacctgggcgtggacgccgagctgctcaccctgatcgaggctgccttcggcgagatcagctccatccacctgcccaccaagaccaagttcaagttcggcgctatgatgaaaagcggaatgttcctgaccctgttcgtgaacaccgtgatcaacattgtgatcgccagcagggtgctgcgggagaggctgaccggcagcccctgcgctgccttcatcggcgacgacaacatcgtgaagggcgtgaaaagcgacaagctgatggccgacaggtgcgccacctggctgaacatggaggtgaagatcatcgacgccgtggtgggcgagaaggccccctacttctgcggcggattcatcctgtgcgacagcgtgaccggcaccgcctgcagggtggccgaccccctgaagaggctgttcaagctgggcaagccactggccgctgacgatgagcacgacgatgacaggcggagggccctgcacgaggaaagcaccaggtggaacagggtgggcatcctgagcgagctgtgcaaggccgtggagagcaggtacgagaccgtgggcaccagcatcatcgtgatggctatgaccacactggccagctccgtcaagagcttctcctacctgaggggggcccctataactctctacggctaacctgaatggactacgacatagtctagtccgccaaggccgccaccatgagagtgacagcccctagaaccttactgcttctgctttggggagctgttgctctgacagagacatgggctggatcttaccacagccccagctacgcctaccaccagttcgagagggggggaggaggctccgggggaggaggctccctgaagatcagccaggccgtgcacgccgcccacgccgagatcaacgaggccggccgggaggtgatcgtgggcattgtcgctggcctggccgtcctcgccgtggtggtgattggagctgtggtcgcagctgttatgtgcagaagaaagtcatccggcggaaagggaggctcctactctcaggctgcttctgctacagtgcctagagctcttatgtgtttatctcagctgggcggcggaggcagcgactacaaggacgacgatgacaagtaaactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 109 - protein, 2512 aa, synthetic construct
  REGION 1..2512: note = Synthetic polypeptide
MEKPVVNVDVDPQSPFVVQLQKSFPQFEVVAQQVTPNDHANARAFSHLASKLIELEVPTTATILDIGSAPARRMFSEHQYHCVCPMRSPEDPDRMMKYASKLAEKACKITNKNLHEKIKDLRTVLDTPDAETPSLCFHNDVTCNMRAEYSVMQDVYINAPGTIYHQAMKGVRTLYWIGFDTTQFMFSAMAGSYPAYNTNWADEKVLEARNIGLCSTKLSEGRTGKLSIMRKKELKPGSRVYFSVGSTLYPEHRASLQSWHLPSVFHLNGKQSYTCRCDTVVSCEGYVVKKITISPGITGETVGYAVTHNSEGFLLCKVTDTVKGERVSFPVCTYIPATICDQMTGIMATDISPDDAQKLLVGLNQRIVINGRTNRNTNTMQNYLLPIIAQGFSKWAKERKDDLDNEKMLGTRERKLTYGCLWAFRTKKVHSFYRPPGTQTCVKVPASFSAFPMSSVWTTSLPMSLRQKLKLALQPKKEEKLLQVSEELVMEAKAAFEDAQEEARAEKLREALPPLVADKGIEAAAEVVCEVEGLQADIGAALVETPRGHVRIIPQANDRMIGQYIVVSPNSVLKNAKLAPAHPLADQVKIITHSGRSGRYAVEPYDAKVLMPAGGAVPWPEFLALSESATLVYNEREFVNRKLYHIAMHGPAKNTEEEQYKVTKAELAETEYVFDVDKKRCVKKEEASGLVLSGELTNPPYHELALEGLKTRPAVPYKVETIGVIGTPGSGKSAIIKSTVTARDLVTSGKKENCREIEADVLRLRGMQITSKTVDSVMLNGCHKAVEVLYVDEAFACHAGALLALIAIVRPRKKVVLCGDPMQCGFFNMMQLKVHFNHPEKDICTKTFYKYISRRCTQPVTAIVSTLHYDGKMKTTNPCKKNIEIDITGATKPKPGDIILTCFRGWVKQLQIDYPGHEVMTAAASQGLTRKGVYAVRQKVNENPLYAITSEHVNVLLTRTEDRLVWKTLQGDPWIKQLTNIPKGNFQATIEDWEAEHKGIIAAINSPTPRANPFSCKTNVCWAKALEPILATAGIVLTGCQWSELFPQFADDKPHSAIYALDVICIKFFGMDLTSGLFSKQSIPLTYHPADSARPVAHWDNSPGTRKYGYDHAIAAELSRRFPVFQLAGKGTQLDLQTGRTRVISAQHNLVPVNRNLPHALVPEYKEKQPGPVEKFLNQFKHHSVLVVSEEKIEAPRKRIEWIAPIGIAGADKNYNLAFGFPPQARYDLVFINIGTKYRNHHFQQCEDHAATLKTLSRSALNCLNPGGTLVVKSYGYADRNSEDVVTALARKFVRVSAARPDCVSSNTEMYLIFRQLDNSRTRQFTPHHLNCVISSVYEGTRDGVGAAPSYRTKRENIADCQEEAVVNAANPLGRPGEGVCRAIYKRWPTSFTDSATETGTARMTVCLGKKVIHAVGPDFRKHPEAEALKLLQNAYHAVADLVNEHNIKSVAIPLLSTGIYAAGKDRLEVSLNCLTTALDRTDADVTIYCLDKKWKERIDAALQLKESVTELKDEDMEIDDELVWIHPDSCLKGRKGFSTTKGKLYSYFEGTKFHQAAKDMAEIKVLFPNDQESNEQLCAYILGETMEAIREKCPVDHNPSSSPPKTLPCLCMYAMTPERVHRLRSNNVKEVTVCSSTPLPKHKIKNVQKVQCTKVVLFNPHTPAFVPARKYIEVPEQPTAPPAQAEEAPEVVATPSPSTADNTSLDVTDISLDMDDSSEGSLFSSFSGSDNSITSMDSWSSGPSSLEIVDRRQVVVADVHAVQEPAPIPPPRLKKMARLAAARKEPTPPASNSSESLHLSFGGVSMSLGSIFDGETARQAAVQPLATGPTDVPMSFGSFSDGEIDELSRRVTESEPVLFGSFEPGEVNSIISSRSAVSFPLRKQRRRRRSRRTEYLTGVGGYIFSTDTGPGHLQKKSVLQNQLTEPTLERNVLERIHAPVLDTSKEEQLKLRYQMMPTEANKSRYQSRKVENQKAITTERLLSGLRLYNSATDQPECYKITYPKPLYSSSVPANYSDPQFAVAVCNNYLHENYPTVASYQITDEYDAYLDMVDGTVACLDTATFCPAKLRSYPKKHEYRAPNIRSAVPSAMQNTLQNVLIAATKRNCNVTQMRELPTLDSATFNVECFRKYACNDEYWEEFARKPIRITTEFVTAYVARLKGPKAAALFAKTYNLVPLQEVPMDRFVMDMKRDVKVTPGTKHTEERPKVQVIQAAEPLATAYLCGIHRELVRRLTAVLLPNIHTLFDMSAEDFDAIIAEHFKQGDPVLETDIASFDKSQDDAMALTGLMILEDLGVDQPLLDLIECAFGEISSTHLPTGTRFKFGAMMKSGMFLTLFVNTVLNVVIASRVLEERLKTSRCAAFIGDDNIIHGVVSDKEMAERCATWLNMEVKIIDAVIGERPPYFCGGFILQDSVTSTACRVADPLKRLFKLGKPLPADDEQDEDRRRALLDETKAWFRVGITGTLAVAVTTRYEVDNITPVLLALRTFAQSKRAFQAIRGEIKHLYGGPK

SEQ ID NO: 110 - protein, 9 aa, synthetic construct
  REGION 1..9: note = Synthetic polypeptide
SPSYVYHQF

SEQ ID NO: 111 - protein, 9 aa, synthetic construct
  REGION 1..9: note = Synthetic polypeptide
SPSYAYHQF

SEQ ID NO: 112 - protein, 9 aa, synthetic construct
  REGION 1..9: note = Synthetic polypeptide
TPHPARIGL

SEQ ID NO: 113 - RNA, 1701 nt, synthetic construct
  misc_feature 1..1701: note = Synthetic polynucleotide
  source 1..1701: mol_type = other RNA
atgaaggctatcctggtggtgctgctctacacctttgccacagccaatgctgacaccctgtgtattggctaccatgccaacaacagcacagacacagtggacacagtgttggagaagaatgtgacagtgacccactctgtgaacctgttggaggacaaacacaatggcaaactgtgtaaactgaggggagtggctccactgcacctgggcaagtgtaacattgctggctggattctgggcaaccctgagtgtgagtccctgagcacagcctcctcctggtcctacattgtggagacaccatcctctgacaatggcacttgttaccctggagacttcattgactatgaggaactgagggaacaactttcctctgtgtcctcctttgagaggtttgagatttttccaaagacctcctcctggccaaaccatgacagcaacaagggagtgacagcagcctgtccacatgctggagccaagtccttctacaagaacctgatttggctggtgaagaagggcaactcctacccaaaactgagcaagtcctacatcaatgacaagggcaaggaggtgctggtgctgtggggcatccaccacccaagcacctctgctgaccaacagtccctctaccagaatgctgacgcctatgtgtttgtgggctccagcagatacagcaagaagttcaagcctgagattgccatcagaccaaaggtgagggatcaggagggcaggatgaactactactggaccctggtggaacctggagacaagattacctttgaggctacaggcaacctggtggtgccaagatatgcctttgctatggagaggaatgctggctctggcatcatcatctctgacacacctgtccatgactgtaacaccacttgtcagacaccaaagggagccatcaacacctccctgccattccagaacatccacccaatcaccattggcaagtgtccaaaatatgtcaagagcaccaaactgagactggctacaggactgaggaacatcccaagcatccagagcaggggactgtttggagccattgctggcttcattgagggaggctggacagggatggtggatggctggtatggctaccaccaccagaatgaacagggctctggctatgctgctgacctgaaaagcacccagaatgccattgatgagattaccaacaaggtgaactctgtgattgagaagatgaacacccagttcacagcagtgggcaaggagttcaaccacttggagaagaggattgagaacctgaacaagaaggtggatgatggcttcctggacatctggacctacaatgctgaactgctggtgctgttggagaatgagaggaccctggactaccatgacagcaatgtgaagaacctctatgagaaggtgaggagccaacttaaaaacaatgccaaggagattggcaatggctgttttgagttctaccacaagtgtgacaacacttgtatggagtctgtgaagaatggcacctatgactacccaaaatactctgaggaggctaaactgaacagggaggagattgatggagtgaaattggagagcaccaggatttaccagatcctggccatctacagcaccgtggccagcagcctggtgctggtggtgagcctgggcgccatcagcttctggatgtgcagcaacggcagcttgcagtgcaggatctgcatctaa

SEQ ID NO: 114 - protein, 566 aa, synthetic construct
  REGION 1..566: note = Synthetic polypeptide
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 115 - RNA, 9911 nt, synthetic construct
  misc_feature 1..9911: note = Synthetic polynucleotide
  source 1..9911: mol_type = other RNA
gataggcggcgcatgagagaagcccagaccaattacctacccaaataggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaatagtcagcatagtacatttcatctgactaatactacaacaccaccaccatgaatagaggattctttaacatgctcggccgccgccccttcccggcccccactgccatgtggaggccgcggagaaggaggcaggcggccccgggaagcggagctactaacttcagcctgctgaagcaggctggagacgtggaggagaaccctggacctgagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctctgaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgctcactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatatcgcaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgacagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagtccctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaaggcgcgccgtttaaacggccggccttaattaagtaacgatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 116 - RNA, 2117 nt, synthetic construct
  misc_feature 1..2117: note = Synthetic polynucleotide
  source 1..2117: mol_type = other RNA
aggaaacttaagtcaacacaacatatacaaaacaaacgaatctcaagcaatcaagcattctacttctattgcagcaatttaaatcatttcttttaaagcaaaagcaattttctgaaaattttcaccatttacgaacgatagccaccatgaaggctatcctggtggtgctgctctacacctttgccacagccaatgctgacaccctgtgtattggctaccatgccaacaacagcacagacacagtggacacagtgttggagaagaatgtgacagtgacccactctgtgaacctgttggaggacaaacacaatggcaaactgtgtaaactgaggggagtggctccactgcacctgggcaagtgtaacattgctggctggattctgggcaaccctgagtgtgagtccctgagcacagcctcctcctggtcctacattgtggagacaccatcctctgacaatggcacttgttaccctggagacttcattgactatgaggaactgagggaacaactttcctctgtgtcctcctttgagaggtttgagatttttccaaagacctcctcctggccaaaccatgacagcaacaagggagtgacagcagcctgtccacatgctggagccaagtccttctacaagaacctgatttggctggtgaagaagggcaactcctacccaaaactgagcaagtcctacatcaatgacaagggcaaggaggtgctggtgctgtggggcatccaccacccaagcacctctgctgaccaacagtccctctaccagaatgctgacgcctatgtgtttgtgggctccagcagatacagcaagaagttcaagcctgagattgccatcagaccaaaggtgagggatcaggagggcaggatgaactactactggaccctggtggaacctggagacaagattacctttgaggctacaggcaacctggtggtgccaagatatgcctttgctatggagaggaatgctggctctggcatcatcatctctgacacacctgtccatgactgtaacaccacttgtcagacaccaaagggagccatcaacacctccctgccattccagaacatccacccaatcaccattggcaagtgtccaaaatatgtcaagagcaccaaactgagactggctacaggactgaggaacatcccaagcatccagagcaggggactgtttggagccattgctggcttcattgagggaggctggacagggatggtggatggctggtatggctaccaccaccagaatgaacagggctctggctatgctgctgacctgaaaagcacccagaatgccattgatgagattaccaacaaggtgaactctgtgattgagaagatgaacacccagttcacagcagtgggcaaggagttcaaccacttggagaagaggattgagaacctgaacaagaaggtggatgatggcttcctggacatctggacctacaatgctgaactgctggtgctgttggagaatgagaggaccctggactaccatgacagcaatgtgaagaacctctatgagaaggtgaggagccaacttaaaaacaatgccaaggagattggcaatggctgttttgagttctaccacaagtgtgacaacacttgtatggagtctgtgaagaatggcacctatgactacccaaaatactctgaggaggctaaactgaacagggaggagattgatggagtgaaattggagagcaccaggatttaccagatcctggccatctacagcaccgtggccagcagcctggtgctggtggtgagcctgggcgccatcagcttctggatgtgcagcaacggcagcttgcagtgcaggatctgcatctaaactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 117 - DNA, 3822 bp, synthetic construct
  misc_feature 1..3822: note = Synthetic polynucleotide
  source 1..3822: mol_type = other DNA
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 118 - protein, 1273 aa, synthetic construct
  REGION 1..1273: note = Synthetic polypeptide
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 119 - DNA, 3822 bp, synthetic construct
  misc_feature 1..3822: note = Synthetic polynucleotide
  source 1..3822: mol_type = other DNA
atgttcgtcttcctggtcctgctgcctctggtctcctcacagtgcgtcaatctgacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcaggacgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcccagaagagcccggagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagtttgacgaggatgactctgaacctgtgctgaagggcgtgaagctgcattacacctaa

SEQ ID NO: 120 - protein, 1273 aa, synthetic construct; identical to SEQ ID NO: 118
  REGION 1..1273: note = Synthetic polypeptide

SEQ ID NO: 121 - DNA, 3822 bp, synthetic construct; identical to SEQ ID NO: 117
  misc_feature 1..3822: note = Synthetic polynucleotide
  source 1..3822: mol_type = other DNA

SEQ ID NO: 122 - DNA, 3822 bp, synthetic construct
  misc_feature 1..3822: note = Synthetic polynucleotide
  source 1..3822: mol_type = other DNA
atgttcgtcttcctggtcctgctgcctctggtctcctcacagtgcgtcaatctgacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagtccaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcaggacgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcccagacgggcccggagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagtttgacgaggatgactctgaacctgtgctgaagggcgtgaagctgcattacacctaa

SEQ ID NO: 123 - protein, 1273 aa, synthetic construct; identical to SEQ ID NO: 118
  REGION 1..1273: note = Synthetic polypeptide

SEQ ID NO: 124 - DNA, 11860 bp, synthetic construct
  misc_feature 1..11860: note = Synthetic polynucleotide
  source 1..11860: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagaccgtgctgaccgccaggaacatcggcctgtgcagcagcgacgtgatggagaggagccggagaggcatgagcatcctgaggaagaaatacctgaagcccagcaacaacgtgctgttcagcgtgggcagcaccatctaccacgagaagagggacctgctcaggagctggcacctgcccagcgtgttccacctgaggggcaagcagaactacacctgcaggtgcgagaccatcgtgagctgcgacggctacgtggtgaagaggatcgccatcagccccggcctgtacggcaagcccagcggctacgccgctacaatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctcgtgggcctgaaccagaggatcgtggtcaacggcaggacccagaggaacaccaacacaatgaagaactacctgctgcccgtggtggcccaggctttcgccaggtgggccaaggagtacaaggaggaccaggaagacgagaggcccctgggcctgagggacaggcagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggaacacaaggagcccagcccactgatcaccgccgaggacgtgcaggaggccaagtgcgctgccgacgaggccaaggaggtgagggaggccgaggaactgagggccgccctgccacccctggctgccgacgtggaggaacccaccctggaagccgacgtggacctgatgctgcaggaggccggcgccggaagcgtggagacacccaggggcctgatcaaggtgaccagctacgacggcgaggacaagatcggcagctacgccgtgctgagcccacaggccgtgctgaagtccgagaagctgagctgcatccacccactggccgagcaggtgatcgtgatcacccacagcggcaggaagggcaggtacgccgtggagccctaccacggcaaggtggtcgtgcccgagggccacgccatccccgtgcaggacttccaggccctgagcgagagcgccaccatcgtgtacaacgagagggagttcgtgaacaggtacctgcaccatatcgccacccacggcggagccctgaacaccgacgaggaatactacaagaccgtgaagcccagcgagcacgacggcgagtacctgtacgacatcgacaggaagcagtgcgtgaagaaagagctggtgaccggcctgggactgaccggcgagctggtggacccacccttccacgagttcgcctacgagagcctgaggaccagacccgccgctccctaccaggtgcccaccatcggcgtgtacggcgtgcccggcagcggaaagagcggcatcatcaagagcgccgtgaccaagaaagacctggtggtcagcgccaagaaagagaactgcgccgagatcatcagggacgtgaagaagatgaaaggcctggacgtgaacgcgcgcaccgtggacagcgtgctgctgaacggctgcaagcaccccgtggagaccctgtacatcgacgaggccttcgcttgccacgccggcaccctgagggccctgatcgccatcatcaggcccaagaaagccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtcgtgagcaccctgttctacgacaagaaaatgaggaccaccaaccccaaggagaccaaaatcgtgatcgacaccacaggcagcaccaagcccaagcaggacgacctgatcctgacctgcttcaggggctgggtgaagcagctgcagatcgactacaagggcaacgagatcatgaccgccgctgccagccagggcctgaccaggaagggcgtgtacgccgtgaggtacaaggtgaacgagaacccactgtacgctcccaccagcgagcacgtgaacgtgctgctgaccaggaccgaggacaggatcgtgtggaagaccctggccggcgacccctggatcaagaccctgaccgccaagtaccccggcaacttcaccgccaccatcgaagagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccacagagcagtggaacaccgtggactacttcgagaccgacaaggcccacagcgccgagatcgtgctgaaccagctgtgcgtgaggttcttcggcctggacctggacagcggcctgttcagcgcccccaccgtgccactgagcatcaggaacaaccactgggacaacagccccagcccaaacatgtacggcctgaacaaggaggtggtcaggcagctgagcaggcggtacccacagctgcccagggccgtggccaccggcagggtgtacgacatgaacaccggcaccctgaggaactacgaccccaggatcaacctggtgcccgtgaacaggcggctgccccacgccctggtgctgcaccacaacgagcacccacagagcgacttcagctccttcgtgagcaagctgaaaggcaggaccgtgctggtcgtgggcgagaagctgagcgtgcccggcaagatggtggactggctgagcgacaggcccgaggccaccttccgggccaggctggacctcggcatccccggcgacgtgcccaagtacgacatcatcttcgtgaacgtcaggaccccatacaagtaccaccattaccagcagtgcgaggaccacgccatcaagctgagcatgctgaccaagaaggcctgcctgcacctgaaccccggaggcacctgcgtgagcatcggctacggctacgccgacagggccagcgagagcatcattggcgccatcgccaggctgttcaagttcagcagggtgtgcaaacccaagagcagcctggaggaaaccgaggtgctgttcgtgttcatcggctacgaccggaaggccaggacccacaacccctacaagctgagcagcaccctgacaaacatctacaccggcagcaggctgcacgaggccggctgcgcccccagctaccacgtggtcaggggcgatatcgccaccgccaccgagggcgtgatcatcaacgctgccaacagcaagggccagcccggaggcggagtgtgcggcgccctgtacaagaagttccccgagagcttcgacctgcagcccatcgaggtgggcaaggccaggctggtgaagggcgccgctaagcacatcatccacgccgtgggccccaacttcaacaaggtgagcgaggtggaaggcgacaagcagctggccgaagcctacgagagcatcgccaagatcgtgaacgacaataactacaagagcgtggccatcccactgctcagcaccggcatcttcagcggcaacaaggacaggctgacccagagcctgaaccacctgctcaccgccctggacaccaccgatgccgacgtggccatctactgcagggacaagaagtgggagatgaccctgaaggaggccgtggccaggcgggaggccgtggaagagatctgcatcagcgacgactccagcgtgaccgagcccgacgccgagctggtgagggtgcaccccaagagctccctggccggcaggaagggctacagcaccagcgacggcaagaccttcagctacctggagggcaccaagttccaccaggccgctaaggacatcgccgagatcaacgctatgtggcccgtggccaccgaggccaacgagcaggtgtgcatgtacatcctgggcgagagcatgtccagcatcaggagcaagtgccccgtggaggaaagcgaggccagcacaccacccagcaccctgccctgcctgtgcatccacgctatgacacccgagagggtgcagcggctgaaggccagcaggcccgagcagatcaccgtgtgcagctccttcccactgcccaagtacaggatcaccggcgtgcagaagatccagtgcagccagcccatcctgttcagcccaaaggtgcccgcctacatccaccccaggaagtacctggtggagaccccacccgtggacgagacacccgagccaagcgccgagaaccagagcaccgagggcacacccgagcagccacccctgatcaccgaggacgagacaaggacccggaccccagagcccatcattatcgaggaagaggaagaggacagcatcagcctgctgagcgacggccccacccaccaggtgctgcaggtggaggccgacatccacggcccacccagcgtgtccagctccagctggagcatcccacacgccagcgacttcgacgtggacagcctgagcatcctggacaccctggagggcgccagcgtgacctccggcgccaccagcgccgagaccaacagctacttcgccaagagcatggagttcctggccaggcccgtgccagctcccaggaccgtgttcaggaacccaccccacccagctcccaggaccaggaccccaagcctggctcccagcagggcctgcagcaggaccagcctggtgagcaccccacccggcgtgaacagggtgatcaccagggaggaactggaggccctgacacccagcaggacccccagcaggtccgtgagcaggactagtctggtgtccaacccacccggcgtgaacagggtgatcaccagggaggaattcgaggccttcgtggcccagcaacagagacggttcgacgccggcgcctacatcttcagcagcgacaccggccagggacacctgcagcaaaagagcgtgaggcagaccgtgctgagcgaggtggtgctggagaggaccgagctggaaatcagctacgcccccaggctggaccaggagaaggaggaactgctcaggaagaaactgcagctgaaccccaccccagccaacaggagcaggtaccagagcaggaaggtggagaacatgaaggccatcaccgccaggcggatcctgcagggcctgggacactacctgaaggccgagggcaaggtggagtgctacaggaccctgcaccccgtgccactgtacagctccagcgtgaacagggccttctccagccccaaggtggccgtggaggcctgcaacgctatgctgaaggagaacttccccaccgtggccagctactgcatcatccccgagtacgacgcctacctggacatggtggacggcgccagctgctgcctggacaccgccagcttctgccccgccaagctgaggagcttccccaagaaacacagctacctggagcccaccatcaggagcgccgtgcccagcgccatccagaacaccctgcagaacgtgctggccgctgccaccaagaggaactgcaacgtgacccagatgagggagctgcccgtgctggacagcgctgccttcaacgtggagtgcttcaagaaatacgcctgcaacaacgagtactgggagaccttcaaggagaaccccatcaggctgaccgaagagaacgtggtgaactacatcaccaagctgaagggccccaaggccgctgccctgttcgctaagacccacaacctgaacatgctgcaggacatcccaatggacaggttcgtgatggacctgaagagggacgtgaaggtgacacccggcaccaagcacaccgaggagaggcccaaggtgcaggtgatccaggccgctgacccactggccaccgcctacctgtgcggcatccacagggagctggtgaggcggctgaacgccgtgctgctgcccaacatccacaccctgttcgacatgagcgccgaggacttcgacgccatcatcgccgagcacttccagcccggcgactgcgtgctggagaccgacatcgccagcttcgacaagagcgaggatgacgctatggccctgaccgctctgatgatcctggaggacctgggcgtggacgccgagctgctcaccctgatcgaggctgccttcggcgagatcagctccatccacctgcccaccaagaccaagttcaagttcggcgctatgatgaaaagcggaatgttcctgaccctgttcgtgaacaccgtgatcaacattgtgatcgccagcagggtgctgcgggagaggctgaccggcagcccctgcgctgccttcatcggcgacgacaacatcgtgaagggcgtgaaaagcgacaagctgatggccgacaggtgcgccacctggctgaacatggaggtgaagatcatcgacgccgtggtgggcgagaaggccccctacttctgcggcggattcatcctgtgcgacagcgtgaccggcaccgcctgcagggtggccgaccccctgaagaggctgttcaagctgggcaagccactggccgctgacgatgagcacgacgatgacaggcggagggccctgcacgaggaaagcaccaggtggaacagggtgggcatcctgagcgagctgtgcaaggccgtggagagcaggtacgagaccgtgggcaccagcatcatcgtgatggctatgaccacactggccagctccgtcaagagcttctcctacctgaggggggcccctataactctctacggctaacctgaatggactacgacatagtctagtccgccaaggccgccaccatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 125 - DNA, 11860 bp, synthetic construct
  misc_feature 1..11860: note = Synthetic polynucleotide
  source 1..11860: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagaccgtgctgaccgccaggaacatcggcctgtgcagcagcgacgtgatggagaggagccggagaggcatgagcatcctgaggaagaaatacctgaagcccagcaacaacgtgctgttcagcgtgggcagcaccatctaccacgagaagagggacctgctcaggagctggcacctgcccagcgtgttccacctgaggggcaagcagaactacacctgcaggtgcgagaccatcgtgagctgcgacggctacgtggtgaagaggatcgccatcagccccggcctgtacggcaagcccagcggctacgccgctacaatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctcgtgggcctgaaccagaggatcgtggtcaacggcaggacccagaggaacaccaacacaatgaagaactacctgctgcccgtggtggcccaggctttcgccaggtgggccaaggagtacaaggaggaccaggaagacgagaggcccctgggcctgagggacaggcagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggaacacaaggagcccagcccactgatcaccgccgaggacgtgcaggaggccaagtgcgctgccgacgaggccaaggaggtgagggaggccgaggaactgagggccgccctgccacccctggctgccgacgtggaggaacccaccctggaagccgacgtggacctgatgctgcaggaggccggcgccggaagcgtggagacacccaggggcctgatcaaggtgaccagctacgacggcgaggacaagatcggcagctacgccgtgctgagcccacaggccgtgctgaagtccgagaagctgagctgcatccacccactggccgagcaggtgatcgtgatcacccacagcggcaggaagggcaggtacgccgtggagccctaccacggcaaggtggtcgtgcccgagggccacgccatccccgtgcaggacttccaggccctgagcgagagcgccaccatcgtgtacaacgagagggagttcgtgaacaggtacctgcaccatatcgccacccacggcggagccctgaacaccgacgaggaatactacaagaccgtgaagcccagcgagcacgacggcgagtacctgtacgacatcgacaggaagcagtgcgtgaagaaagagctggtgaccggcctgggactgaccggcgagctggtggacccacccttccacgagttcgcctacgagagcctgaggaccagacccgccgctccctaccaggtgcccaccatcggcgtgtacggcgtgcccggcagcggaaagagcggcatcatcaagagcgccgtgaccaagaaagacctggtggtcagcgccaagaaagagaactgcgccgagatcatcagggacgtgaagaagatgaaaggcctggacgtgaacgcgcgcaccgtggacagcgtgctgctgaacggctgcaagcaccccgtggagaccctgtacatcgacgaggccttcgcttgccacgccggcaccctgagggccctgatcgccatcatcaggcccaagaaagccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtcgtgagcaccctgttctacgacaagaaaatgaggaccaccaaccccaaggagaccaaaatcgtgatcgacaccacaggcagcaccaagcccaagcaggacgacctgatcctgacctgcttcaggggctgggtgaagcagctgcagatcgactacaagggcaacgagatcatgaccgccgctgccagccagggcctgaccaggaagggcgtgtacgccgtgaggtacaaggtgaacgagaacccactgtacgctcccaccagcgagcacgtgaacgtgctgctgaccaggaccgaggacaggatcgtgtggaagaccctggccggcgacccctggatcaagaccctgaccgccaagtaccccggcaacttcaccgccaccatcgaagagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccacagagcagtggaacaccgtggactacttcgagaccgacaaggcccacagcgccgagatcgtgctgaaccagctgtgcgtgaggttcttcggcctggacctggacagcggcctgttcagcgcccccaccgtgccactgagcatcaggaacaaccactgggacaacagccccagcccaaacatgtacggcctgaacaaggaggtggtcaggcagctgagcaggcggtacccacagctgcccagggccgtggccaccggcagggtgtacgacatgaacaccggcaccctgaggaactacgaccccaggatcaacctggtgcccgtgaacaggcggctgccccacgccctggtgctgcaccacaacgagcacccacagagcgacttcagctccttcgtgagcaagctgaaaggcaggaccgtgctggtcgtgggcgagaagctgagcgtgcccggcaagatggtggactggctgagcgacaggcccgaggccaccttccgggccaggctggacctcggcatccccggcgacgtgcccaagtacgacatcatcttcgtgaacgtcaggaccccatacaagtaccaccattaccagcagtgcgaggaccacgccatcaagctgagcatgctgaccaagaaggcctgcctgcacctgaaccccggaggcacctgcgtgagcatcggctacggctacgccgacagggccagcgagagcatcattggcgccatcgccaggctgttcaagttcagcagggtgtgcaaacccaagagcagcctggaggaaaccgaggtgctgttcgtgttcatcggctacgaccggaaggccaggacccacaacccctacaagctgagcagcaccctgacaaacatctacaccggcagcaggctgcacgaggccggctgcgcccccagctaccacgtggtcaggggcgatatcgccaccgccaccgagggcgtgatcatcaacgctgccaacagcaagggccagcccggaggcggagtgtgcggcgccctgtacaagaagttccccgagagcttcgacctgcagcccatcgaggtgggcaaggccaggctggtgaagggcgccgctaagcacatcatccacgccgtgggccccaacttcaacaaggtgagcgaggtggaaggcgacaagcagctggccgaagcctacgagagcatcgccaagatcgtgaacgacaataactacaagagcgtggccatcccactgctcagcaccggcatcttcagcggcaacaaggacaggctgacccagagcctgaaccacctgctcaccgccctggacaccaccgatgccgacgtggccatctactgcagggacaagaagtgggagatgaccctgaaggaggccgtggccaggcgggaggccgtggaagagatctgcatcagcgacgactccagcgtgaccgagcccgacgccgagctggtgagggtgcaccccaagagctccctggccggcaggaagggctacagcaccagcgacggcaagaccttcagctacctggagggcaccaagttccaccaggccgctaaggacatcgccgagatcaacgctatgtggcccgtggccaccgaggccaacgagcaggtgtgcatgtacatcctgggcgagagcatgtccagcatcaggagcaagtgccccgtggaggaaagcgaggccagcacaccacccagcaccctgccctgcctgtgcatccacgctatgacacccgagagggtgcagcggctgaaggccagcaggcccgagcagatcaccgtgtgcagctccttcccactgcccaagtacaggatcaccggcgtgcagaagatccagtgcagccagcccatcctgttcagcccaaaggtgcccgcctacatccaccccaggaagtacctggtggagaccccacccgtggacgagacacccgagccaagcgccgagaaccagagcaccgagggcacacccgagcagccacccctgatcaccgaggacgagacaaggacccggaccccagagcccatcattatcgaggaagaggaagaggacagcatcagcctgctgagcgacggccccacccaccaggtgctgcaggtggaggccgacatccacggcccacccagcgtgtccagctccagctggagcatcccacacgccagcgacttcgacgtggacagcctgagcatcctggacaccctggagggcgccagcgtgacctccggcgccaccagcgccgagaccaacagctacttcgccaagagcatggagttcctggccaggcccgtgccagctcccaggaccgtgttcaggaacccaccccacccagctcccaggaccaggaccccaagcctggctcccagcagggcctgcagcaggaccagcctggtgagcaccccacccggcgtgaacagggtgatcaccagggaggaactggaggccctgacacccagcaggacccccagcaggtccgtgagcaggactagtctggtgtccaacccacccggcgtgaacagggtgatcaccagggaggaattcgaggccttcgtggcccagcaacagagacggttcgacgccggcgcctacatcttcagcagcgacaccggccagggacacctgcagcaaaagagcgtgaggcagaccgtgctgagcgaggtggtgctggagaggaccgagctggaaatcagctacgcccccaggctggaccaggagaaggaggaactgctcaggaagaaactgcagctgaaccccaccccagccaacaggagcaggtaccagagcaggaaggtggagaacatgaaggccatcaccgccaggcggatcctgcagggcctgggacactacctgaaggccgagggcaaggtggagtgctacaggaccctgcaccccgtgccactgtacagctccagcgtgaacagggccttctccagccccaaggtggccgtggaggcctgcaacgctatgctgaaggagaacttccccaccgtggccagctactgcatcatccccgagtacgacgcctacctggacatggtggacggcgccagctgctgcctggacaccgccagcttctgccccgccaagctgaggagcttccccaagaaacacagctacctggagcccaccatcaggagcgccgtgcccagcgccatccagaacaccctgcagaacgtgctggccgctgccaccaagaggaactgcaacgtgacccagatgagggagctgcccgtgctggacagcgctgccttcaacgtggagtgcttcaagaaatacgcctgcaacaacgagtactgggagaccttcaaggagaaccccatcaggctgaccgaagagaacgtggtgaactacatcaccaagctgaagggccccaaggccgctgccctgttcgctaagacccacaacctgaacatgctgcaggacatcccaatggacaggttcgtgatggacctgaagagggacgtgaaggtgacacccggcaccaagcacaccgaggagaggcccaaggtgcaggtgatccaggccgctgacccactggccaccgcctacctgtgcggcatccacagggagctggtgaggcggctgaacgccgtgctgctgcccaacatccacaccctgttcgacatgagcgccgaggacttcgacgccatcatcgccgagcacttccagcccggcgactgcgtgctggagaccgacatcgccagcttcgacaagagcgaggatgacgctatggccctgaccgctctgatgatcctggaggacctgggcgtggacgccgagctgctcaccctgatcgaggctgccttcggcgagatcagctccatccacctgcccaccaagaccaagttcaagttcggcgctatgatgaaaagcggaatgttcctgaccctgttcgtgaacaccgtgatcaacattgtgatcgccagcagggtgctgcgggagaggctgaccggcagcccctgcgctgccttcatcggcgacgacaacatcgtgaagggcgtgaaaagcgacaagctgatggccgacaggtgcgccacctggctgaacatggaggtgaagatcatcgacgccgtggtgggcgagaaggccccctacttctgcggcggattcatcctgtgcgacagcgtgaccggcaccgcctgcagggtggccgaccccctgaagaggctgttcaagctgggcaagccactggccgctgacgatgagcacgacgatgacaggcggagggccctgcacgaggaaagcaccaggtggaacagggtgggcatcctgagcgagctgtgcaaggccgtggagagcaggtacgagaccgtgggcaccagcatcatcgtgatggctatgaccacactggccagctccgtcaagagcttctcctacctgaggggggcccctataactctctacggctaacctgaatggactacgacatagtctagtccgccaaggccgccaccatgttcgtcttcctggtcctgctgcctctggtctcctcacagtgcgtcaatctgacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagtccaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcaggacgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcccagacgggcccggagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagtttgacgaggatgactctgaacctgtgctgaagggcgtgaagctgcattacacctaaactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 126 - DNA, 4238 bp, synthetic construct
  misc_feature 1..4238: note = Synthetic polynucleotide
  source 1..4238: mol_type = other DNA
aggaaacttaagtcaacacaacatatacaaaacaaacgaatctcaagcaatcaagcattctacttctattgcagcaatttaaatcatttcttttaaagcaaaagcaattttctgaaaattttcaccatttacgaacgatagccaccatgttcgtcttcctggtcctgctgcctctggtctcctcacagtgcgtcaatctgacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagtccaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcaggacgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcccagacgggcccggagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagtttgacgaggatgactctgaacctgtgctgaagggcgtgaagctgcattacacctaaactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 127 - DNA, 10508 bp, synthetic construct
  misc_feature 1..10508: note = Synthetic polynucleotide
  source 1..10508: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagaccgtgctgaccgccaggaacatcggcctgtgcagcagcgacgtgatggagaggagccggagaggcatgagcatcctgaggaagaaatacctgaagcccagcaacaacgtgctgttcagcgtgggcagcaccatctaccacgagaagagggacctgctcaggagctggcacctgcccagcgtgttccacctgaggggcaagcagaactacacctgcaggtgcgagaccatcgtgagctgcgacggctacgtggtgaagaggatcgccatcagccccggcctgtacggcaagcccagcggctacgccgctacaatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctcgtgggcctgaaccagaggatcgtggtcaacggcaggacccagaggaacaccaacacaatgaagaactacctgctgcccgtggtggcccaggctttcgccaggtgggccaaggagtacaaggaggaccaggaagacgagaggcccctgggcctgagggacaggcagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggaacacaaggagcccagcccactgatcaccgccgaggacgtgcaggaggccaagtgcgctgccgacgaggccaaggaggtgagggaggccgaggaactgagggccgccctgccacccctggctgccgacgtggaggaacccaccctggaagccgacgtggacctgatgctgcaggaggccggcgccggaagcgtggagacacccaggggcctgatcaaggtgaccagctacgacggcgaggacaagatcggcagctacgccgtgctgagcccacaggccgtgctgaagtccgagaagctgagctgcatccacccactggccgagcaggtgatcgtgatcacccacagcggcaggaagggcaggtacgccgtggagccctaccacggcaaggtggtcgtgcccgagggccacgccatccccgtgcaggacttccaggccctgagcgagagcgccaccatcgtgtacaacgagagggagttcgtgaacaggtacctgcaccatatcgccacccacggcggagccctgaacaccgacgaggaatactacaagaccgtgaagcccagcgagcacgacggcgagtacctgtacgacatcgacaggaagcagtgcgtgaagaaagagctggtgaccggcctgggactgaccggcgagctggtggacccacccttccacgagttcgcctacgagagcctgaggaccagacccgccgctccctaccaggtgcccaccatcggcgtgtacggcgtgcccggcagcggaaagagcggcatcatcaagagcgccgtgaccaagaaagacctggtggtcagcgccaagaaagagaactgcgccgagatcatcagggacgtgaagaagatgaaaggcctggacgtgaacgcgcgcaccgtggacagcgtgctgctgaacggctgcaagcaccccgtggagaccctgtacatcgacgaggccttcgcttgccacgccggcaccctgagggccctgatcgccatcatcaggcccaagaaagccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtcgtgagcaccctgttctacgacaagaaaatgaggaccaccaaccccaaggagaccaaaatcgtgatcgacaccacaggcagcaccaagcccaagcaggacgacctgatcctgacctgcttcaggggctgggtgaagcagctgcagatcgactacaagggcaacgagatcatgaccgccgctgccagccagggcctgaccaggaagggcgtgtacgccgtgaggtacaaggtgaacgagaacccactgtacgctcccaccagcgagcacgtgaacgtgctgctgaccaggaccgaggacaggatcgtgtggaagaccctggccggcgacccctggatcaagaccctgaccgccaagtaccccggcaacttcaccgccaccatcgaagagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccacagagcagtggaacaccgtggactacttcgagaccgacaaggcccacagcgccgagatcgtgctgaaccagctgtgcgtgaggttcttcggcctggacctggacagcggcctgttcagcgcccccaccgtgccactgagcatcaggaacaaccactgggacaacagccccagcccaaacatgtacggcctgaacaaggaggtggtcaggcagctgagcaggcggtacccacagctgcccagggccgtggccaccggcagggtgtacgacatgaacaccggcaccctgaggaactacgaccccaggatcaacctggtgcccgtgaacaggcggctgccccacgccctggtgctgcaccacaacgagcacccacagagcgacttcagctccttcgtgagcaagctgaaaggcaggaccgtgctggtcgtgggcgagaagctgagcgtgcccggcaagatggtggactggctgagcgacaggcccgaggccaccttccgggccaggctggacctcggcatccccggcgacgtgcccaagtacgacatcatcttcgtgaacgtcaggaccccatacaagtaccaccattaccagcagtgcgaggaccacgccatcaagctgagcatgctgaccaagaaggcctgcctgcacctgaaccccggaggcacctgcgtgagcatcggctacggctacgccgacagggccagcgagagcatcattggcgccatcgccaggctgttcaagttcagcagggtgtgcaaacccaagagcagcctggaggaaaccgaggtgctgttcgtgttcatcggctacgaccggaaggccaggacccacaacccctacaagctgagcagcaccctgacaaacatctacaccggcagcaggctgcacgaggccggctgcgcccccagctaccacgtggtcaggggcgatatcgccaccgccaccgagggcgtgatcatcaacgctgccaacagcaagggccagcccggaggcggagtgtgcggcgccctgtacaagaagttccccgagagcttcgacctgcagcccatcgaggtgggcaaggccaggctggtgaagggcgccgctaagcacatcatccacgccgtgggccccaacttcaacaaggtgagcgaggtggaaggcgacaagcagctggccgaagcctacgagagcatcgccaagatcgtgaacgacaataactacaagagcgtggccatcccactgctcagcaccggcatcttcagcggcaacaaggacaggctgacccagagcctgaaccacctgctcaccgccctggacaccaccgatgccgacgtggccatctactgcagggacaagaagtgggagatgaccctgaaggaggccgtggccaggcgggaggccgtggaagagatctgcatcagcgacgactccagcgtgaccgagcccgacgccgagctggtgagggtgcaccccaagagctccctggccggcaggaagggctacagcaccagcgacggcaagaccttcagctacctggagggcaccaagttccaccaggccgctaaggacatcgccgagatcaacgctatgtggcccgtggccaccgaggccaacgagcaggtgtgcatgtacatcctgggcgagagcatgtccagcatcaggagcaagtgccccgtggaggaaagcgaggccagcacaccacccagcaccctgccctgcctgtgcatccacgctatgacacccgagagggtgcagcggctgaaggccagcaggcccgagcagatcaccgtgtgcagctccttcccactgcccaagtacaggatcaccggcgtgcagaagatccagtgcagccagcccatcctgttcagcccaaaggtgcccgcctacatccaccccaggaagtacctggtggagaccccacccgtggacgagacacccgagccaagcgccgagaaccagagcaccgagggcacacccgagcagccacccctgatcaccgaggacgagacaaggacccggaccccagagcccatcattatcgaggaagaggaagaggacagcatcagcctgctgagcgacggccccacccaccaggtgctgcaggtggaggccgacatccacggcccacccagcgtgtccagctccagctggagcatcccacacgccagcgacttcgacgtggacagcctgagcatcctggacaccctggagggcgccagcgtgacctccggcgccaccagcgccgagaccaacagctacttcgccaagagcatggagttcctggccaggcccgtgccagctcccaggaccgtgttcaggaacccaccccacccagctcccaggaccaggaccccaagcctggctcccagcagggcctgcagcaggaccagcctggtgagcaccccacccggcgtgaacagggtgatcaccagggaggaactggaggccctgacacccagcaggacccccagcaggtccgtgagcaggactagtctggtgtccaacccacccggcgtgaacagggtgatcaccagggaggaattcgaggccttcgtggcccagcaacagagacggttcgacgccggcgcctacatcttcagcagcgacaccggccagggacacctgcagcaaaagagcgtgaggcagaccgtgctgagcgaggtggtgctggagaggaccgagctggaaatcagctacgcccccaggctggaccaggagaaggaggaactgctcaggaagaaactgcagctgaaccccaccccagccaacaggagcaggtaccagagcaggaaggtggagaacatgaaggccatcaccgccaggcggatcctgcagggcctgggacactacctgaaggccgagggcaaggtggagtgctacaggaccctgcaccccgtgccactgtacagctccagcgtgaacagggccttctccagccccaaggtggccgtggaggcctgcaacgctatgctgaaggagaacttccccaccgtggccagctactgcatcatccccgagtacgacgcctacctggacatggtggacggcgccagctgctgcctggacaccgccagcttctgccccgccaagctgaggagcttccccaagaaacacagctacctggagcccaccatcaggagcgccgtgcccagcgccatccagaacaccctgcagaacgtgctggccgctgccaccaagaggaactgcaacgtgacccagatgagggagctgcccgtgctggacagcgctgccttcaacgtggagtgcttcaagaaatacgcctgcaacaacgagtactgggagaccttcaaggagaaccccatcaggctgaccgaagagaacgtggtgaactacatcaccaagctgaagggccccaaggccgctgccctgttcgctaagacccacaacctgaacatgctgcaggacatcccaatggacaggttcgtgatggacctgaagagggacgtgaaggtgacacccggcaccaagcacaccgaggagaggcccaaggtgcaggtgatccaggccgctgacccactggccaccgcctacctgtgcggcatccacagggagctggtgaggcggctgaacgccgtgctgctgcccaacatccacaccctgttcgacatgagcgccgaggacttcgacgccatcatcgccgagcacttccagcccggcgactgcgtgctggagaccgacatcgccagcttcgacaagagcgaggatgacgctatggccctgaccgctctgatgatcctggaggacctgggcgtggacgccgagctgctcaccctgatcgaggctgccttcggcgagatcagctccatccacctgcccaccaagaccaagttcaagttcggcgctatgatgaaaagcggaatgttcctgaccctgttcgtgaacaccgtgatcaacattgtgatcgccagcagggtgctgcgggagaggctgaccggcagcccctgcgctgccttcatcggcgacgacaacatcgtgaagggcgtgaaaagcgacaagctgatggccgacaggtgcgccacctggctgaacatggaggtgaagatcatcgacgccgtggtgggcgagaaggccccctacttctgcggcggattcatcctgtgcgacagcgtgaccggcaccgcctgcagggtggccgaccccctgaagaggctgttcaagctgggcaagccactggccgctgacgatgagcacgacgatgacaggcggagggccctgcacgaggaaagcaccaggtggaacagggtgggcatcctgagcgagctgtgcaaggccgtggagagcaggtacgagaccgtgggcaccagcatcatcgtgatggctatgaccacactggccagctccgtcaagagcttctcctacctgaggggggcccctataactctctacggctaacctgaatggactacgacatagtctagccaccatgagcaagatctacatcgacgagcggagcaacgccgagatcgtgtgcgaggccatcaagaccatcggcatcgagggcgccaccgccgcccagctgaccaggcagctgaacatggagaagcgggaggtgaacaaggccctgtacgacctgcagaggagcgctatggtgtactccagcgacgacatccctccccggtggttcatgaccaccgaggccgacaagcccgacgccgacgctatggccgacgtgatcatcgacgacgtgagcagggagaagtccatgagggaggaccacaagagcttcgacgacgtgatccccgccaagaagatcatcgactggaagggcgccaaccccgtgaccgtgatcaacgagtactgccagatcaccaggagggactggagcttccggatcgagagcgtgggccccagcaacagccccaccttctacgcctgcgtggacatcgacggcagggtgttcgacaaggccgacggcaagagcaagcgggacgccaagaacaacgccgccaagctggccgtggacaagctgctgggctacgtgatcatccggttctaaactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctagagctccgtcaagagcttctcctacctgaggggggcccctataactctctacggctaacctgaatggactacgacatagtctagccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgacagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagtccctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 128 - RNA, 11075 nt, synthetic construct
  misc_feature 1..11075: note = Synthetic polynucleotide
  source 1..11075: mol_type = other RNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagaccgtgctgaccgccaggaacatcggcctgtgcagcagcgacgtgatggagaggagccggagaggcatgagcatcctgaggaagaaatacctgaagcccagcaacaacgtgctgttcagcgtgggcagcaccatctaccacgagaagagggacctgctcaggagctggcacctgcccagcgtgttccacctgaggggcaagcagaactacacctgcaggtgcgagaccatcgtgagctgcgacggctacgtggtgaagaggatcgccatcagccccggcctgtacggcaagcccagcggctacgccgctacaatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctcgtgggcctgaaccagaggatcgtggtcaacggcaggacccagaggaacaccaacacaatgaagaactacctgctgcccgtggtggcccaggctttcgccaggtgggccaaggagtacaaggaggaccaggaagacgagaggcccctgggcctgagggacaggcagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggaacacaaggagcccagcccactgatcaccgccgaggacgtgcaggaggccaagtgcgctgccgacgaggccaaggaggtgagggaggccgaggaactgagggccgccctgccacccctggctgccgacgtggaggaacccaccctggaagccgacgtggacctgatgctgcaggaggccggcgccggaagcgtggagacacccaggggcctgatcaaggtgaccagctacgacggcgaggacaagatcggcagctacgccgtgctgagcccacaggccgtgctgaagtccgagaagctgagctgcatccacccactggccgagcaggtgatcgtgatcacccacagcggcaggaagggcaggtacgccgtggagccctaccacggcaaggtggtcgtgcccgagggccacgccatccccgtgcaggacttccaggccctgagcgagagcgccaccatcgtgtacaacgagagggagttcgtgaacaggtacctgcaccatatcgccacccacggcggagccctgaacaccgacgaggaatactacaagaccgtgaagcccagcgagcacgacggcgagtacctgtacgacatcgacaggaagcagtgcgtgaagaaagagctggtgaccggcctgggactgaccggcgagctggtggacccacccttccacgagttcgcctacgagagcctgaggaccagacccgccgctccctaccaggtgcccaccatcggcgtgtacggcgtgcccggcagcggaaagagcggcatcatcaagagcgccgtgaccaagaaagacctggtggtcagcgccaagaaagagaactgcgccgagatcatcagggacgtgaagaagatgaaaggcctggacgtgaacgcgcgcaccgtggacagcgtgctgctgaacggctgcaagcaccccgtggagaccctgtacatcgacgaggccttcgcttgccacgccggcaccctgagggccctgatcgccatcatcaggcccaagaaagccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtcgtgagcaccctgttctacgacaagaaaatgaggaccaccaaccccaaggagaccaaaatcgtgatcgacaccacaggcagcaccaagcccaagcaggacgacctgatcctgacctgcttcaggggctgggtgaagcagctgcagatcgactacaagggcaacgagatcatgaccgccgctgccagccagggcctgaccaggaagggcgtgtacgccgtgaggtacaaggtgaacgagaacccactgtacgctcccaccagcgagcacgtgaacgtgctgctgaccaggaccgaggacaggatcgtgtggaagaccctggccggcgacccctggatcaagaccctgaccgccaagtaccccggcaacttcaccgccaccatcgaagagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccacagagcagtggaacaccgtggactacttcgagaccgacaaggcccacagcgccgagatcgtgctgaaccagctgtgcgtgaggttcttcggcctggacctggacagcggcctgttcagcgcccccaccgtgccactgagcatcaggaacaaccactgggacaacagccccagcccaaacatgtacggcctgaacaaggaggtggtcaggcagctgagcaggcggtacccacagctgcccagggccgtggccaccggcagggtgtacgacatgaacaccggcaccctgaggaactacgaccccaggatcaacctggtgcccgtgaacaggcggctgccccacgccctggtgctgcaccacaacgagcacccacagagcgacttcagctccttcgtgagcaagctgaaaggcaggaccgtgctggtcgtgggcgagaagctgagcgtgcccggcaagatggtggactggctgagcgacaggcccgaggccaccttccgggccaggctggacctcggcatccccggcgacgtgcccaagtacgacatcatcttcgtgaacgtcaggaccccatacaagtaccaccattaccagcagtgcgaggaccacgccatcaagctgagcatgctgaccaagaaggcctgcctgcacctgaaccccggaggcacctgcgtgagcatcggctacggctacgccgacagggccagcgagagcatcattggcgccatcgccaggctgttcaagttcagcagggtgtgcaaacccaagagcagcctggaggaaaccgaggtgctgttcgtgttcatcggctacgaccggaaggccaggacccacaacccctacaagctgagcagcaccctgacaaacatctacaccggcagcaggctgcacgaggccggctgcgcccccagctaccacgtggtcaggggcgatatcgccaccgccaccgagggcgtgatcatcaacgctgccaacagcaagggccagcccggaggcggagtgtgcggcgccctgtacaagaagttccccgagagcttcgacctgcagcccatcgaggtgggcaaggccaggctggtgaagggcgccgctaagcacatcatccacgccgtgggccccaacttcaacaaggtgagcgaggtggaaggcgacaagcagctggccgaagcctacgagagcatcgccaagatcgtgaacgacaataactacaagagcgtggccatcccactgctcagcaccggcatcttcagcggcaacaaggacaggctgacccagagcctgaaccacctgctcaccgccctggacaccaccgatgccgacgtggccatctactgcagggacaagaagtgggagatgaccctgaaggaggccgtggccaggcgggaggccgtggaagagatctgcatcagcgacgactccagcgtgaccgagcccgacgccgagctggtgagggtgcaccccaagagctccctggccggcaggaagggctacagcaccagcgacggcaagaccttcagctacctggagggcaccaagttccaccaggccgctaaggacatcgccgagatcaacgctatgtggcccgtggccaccgaggccaacgagcaggtgtgcatgtacatcctgggcgagagcatgtccagcatcaggagcaagtgccccgtggaggaaagcgaggccagcacaccacccagcaccctgccctgcctgtgcatccacgctatgacacccgagagggtgcagcggctgaaggccagcaggcccgagcagatcaccgtgtgcagctccttcccactgcccaagtacaggatcaccggcgtgcagaagatccagtgcagccagcccatcctgttcagcccaaaggtgcccgcctacatccaccccaggaagtacctggtggagaccccacccgtggacgagacacccgagccaagcgccgagaaccagagcaccgagggcacacccgagcagccacccctgatcaccgaggacgagacaaggacccggaccccagagcccatcattatcgaggaagaggaagaggacagcatcagcctgctgagcgacggccccacccaccaggtgctgcaggtggaggccgacatccacggcccacccagcgtgtccagctccagctggagcatcccacacgccagcgacttcgacgtggacagcctgagcatcctggacaccctggagggcgccagcgtgacctccggcgccaccagcgccgagaccaacagctacttcgccaagagcatggagttcctggccaggcccgtgccagctcccaggaccgtgttcaggaacccaccccacccagctcccaggaccaggaccccaagcctggctcccagcagggcctgcagcaggaccagcctggtgagcaccccacccggcgtgaacagggtgatcaccagggaggaactggaggccctgacacccagcaggacccccagcaggtccgtgagcaggactagtctggtgtccaacccacccggcgtgaacagggtgatcaccagggaggaattcgaggccttcgtggcccagcaacagagacggttcgacgccggcgcctacatcttcagcagcgacaccggccagggacacctgcagcaaaagagcgtgaggcagaccgtgctgagcgaggtggtgctggagaggaccgagctggaaatcagctacgcccccaggctggaccaggagaaggaggaactgctcaggaagaaactgcagctgaaccccaccccagccaacaggagcaggtaccagagcaggaaggtggagaacatgaaggccatcaccgccaggcggatcctgcagggcctgggacactacctgaaggccgagggcaaggtggagtgctacaggaccctgcaccccgtgccactgtacagctccagcgtgaacagggccttctccagccccaaggtggccgtggaggcctgcaacgctatgctgaaggagaacttccccaccgtggccagctactgcatcatccccgagtacgacgcctacctggacatggtggacggcgccagctgctgcctggacaccgccagcttctgccccgccaagctgaggagcttccccaagaaacacagctacctggagcccaccatcaggagcgccgtgcccagcgccatccagaacaccctgcagaacgtgctggccgctgccaccaagaggaactgcaacgtgacccagatgagggagctgcccgtgctggacagcgctgccttcaacgtggagtgcttcaagaaatacgcctgcaacaacgagtactgggagaccttcaaggagaaccccatcaggctgaccgaagagaacgtggtgaactacatcaccaagctgaagggccccaaggccgctgccctgttcgctaagacccacaacctgaacatgctgcaggacatcccaatggacaggttcgtgatggacctgaagagggacgtgaaggtgacacccggcaccaagcacaccgaggagaggcccaaggtgcaggtgatccaggccgctgacccactggccaccgcctacctgtgcggcatccacagggagctggtgaggcggctgaacgccgtgctgctgcccaacatccacaccctgttcgacatgagcgccgaggacttcgacgccatcatcgccgagcacttccagcccggcgactgcgtgctggagaccgacatcgccagcttcgacaagagcgaggatgacgctatggccctgaccgctctgatgatcctggaggacctgggcgtggacgccgagctgctcaccctgatcgaggctgccttcggcgagatcagctccatccacctgcccaccaagaccaagttcaagttcggcgctatgatgaaaagcggaatgttcctgaccctgttcgtgaacaccgtgatcaacattgtgatcgccagcagggtgctgcgggagaggctgaccggcagcccctgcgctgccttcatcggcgacgacaacatcgtgaagggcgtgaaaagcgacaagctgatggccgacaggtgcgccacctggctgaacatggaggtgaagatcatcgacgccgtggtgggcgagaaggccccctacttctgcggcggattcatcctgtgcgacagcgtgaccggcaccgcctgcagggtggccgaccccctgaagaggctgttcaagctgggcaagccactggccgctgacgatgagcacgacgatgacaggcggagggccctgcacgaggaaagcaccaggtggaacagggtgggcatcctgagcgagctgtgcaaggccgtggagagcaggtacgagaccgtgggcaccagcatcatcgtgatggctatgaccacactggccagctccgtcaagagcttctcctacctgaggggggcccctataactctctacggctaacctgaatggactacgacatagtctagtccgccaaggccgccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgacagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagtccctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaactcgagccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgagcaagatctacatcgacgagcggagcaacgccgagatcgtgtgcgaggccatcaagaccatcggcatcgagggcgccaccgccgcccagctgaccaggcagctgaacatggagaagcgggaggtgaacaaggccctgtacgacctgcagaggagcgctatggtgtactccagcgacgacatccctccccggtggttcatgaccaccgaggccgacaagcccgacgccgacgctatggccgacgtgatcatcgacgacgtgagcagggagaagtccatgagggaggaccacaagagcttcgacgacgtgatccccgccaagaagatcatcgactggaagggcgccaaccccgtgaccgtgatcaacgagtactgccagatcaccaggagggactggagcttccggatcgagagcgtgggccccagcaacagccccaccttctacgcctgcgtggacatcgacggcagggtgttcgacaaggccgacggcaagagcaagcgggacgccaagaacaacgccgccaagctggccgtggacaagctgctgggctacgtgatcatccggttctaaacgtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 129 - RNA, 10851 nt, synthetic construct
  misc_feature 1..10851: note = Synthetic polynucleotide
  source 1..10851: mol_type = other RNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagaccgtgctgaccgccaggaacatcggcctgtgcagcagcgacgtgatggagaggagccggagaggcatgagcatcctgaggaagaaatacctgaagcccagcaacaacgtgctgttcagcgtgggcagcaccatctaccacgagaagagggacctgctcaggagctggcacctgcccagcgtgttccacctgaggggcaagcagaactacacctgcaggtgcgagaccatcgtgagctgcgacggctacgtggtgaagaggatcgccatcagccccggcctgtacggcaagcccagcggctacgccgctacaatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctcgtgggcctgaaccagaggatcgtggtcaacggcaggacccagaggaacaccaacacaatgaagaactacctgctgcccgtggtggcccaggctttcgccaggtgggccaaggagtacaaggaggaccaggaagacgagaggcccctgggcctgagggacaggcagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggaacacaaggagcccagcccactgatcaccgccgaggacgtgcaggaggccaagtgcgctgccgacgaggccaaggaggtgagggaggccgaggaactgagggccgccctgccacccctggctgccgacgtggaggaacccaccctggaagccgacgtggacctgatgctgcaggaggccggcgccggaagcgtggagacacccaggggcctgatcaaggtgaccagctacgacggcgaggacaagatcggcagctacgccgtgctgagcccacaggccgtgctgaagtccgagaagctgagctgcatccacccactggccgagcaggtgatcgtgatcacccacagcggcaggaagggcaggtacgccgtggagccctaccacggcaaggtggtcgtgcccgagggccacgccatccccgtgcaggacttccaggccctgagcgagagcgccaccatcgtgtacaacgagagggagttcgtgaacaggtacctgcaccatatcgccacccacggcggagccctgaacaccgacgaggaatactacaagaccgtgaagcccagcgagcacgacggcgagtacctgtacgacatcgacaggaagcagtgcgtgaagaaagagctggtgaccggcctgggactgaccggcgagctggtggacccacccttccacgagttcgcctacgagagcctgaggaccagacccgccgctccctaccaggtgcccaccatcggcgtgtacggcgtgcccggcagcggaaagagcggcatcatcaagagcgccgtgaccaagaaagacctggtggtcagcgccaagaaagagaactgcgccgagatcatcagggacgtgaagaagatgaaaggcctggacgtgaacgcgcgcaccgtggacagcgtgctgctgaacggctgcaagcaccccgtggagaccctgtacatcgacgaggccttcgcttgccacgccggcaccctgagggccctgatcgccatcatcaggcccaagaaagccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtcgtgagcaccctgttctacgacaagaaaatgaggaccaccaaccccaaggagaccaaaatcgtgatcgacaccacaggcagcaccaagcccaagcaggacgacctgatcctgacctgcttcaggggctgggtgaagcagctgcagatcgactacaagggcaacgagatcatgaccgccgctgccagccagggcctgaccaggaagggcgtgtacgccgtgaggtacaaggtgaacgagaacccactgtacgctcccaccagcgagcacgtgaacgtgctgctgaccaggaccgaggacaggatcgtgtggaagaccctggccggcgacccctggatcaagaccctgaccgccaagtaccccggcaacttcaccgccaccatcgaagagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccacagagcagtggaacaccgtggactacttcgagaccgacaaggcccacagcgccgagatcgtgctgaaccagctgtgcgtgaggttcttcggcctggacctggacagcggcctgttcagcgcccccaccgtgccactgagcatcaggaacaaccactgggacaacagccccagcccaaacatgtacggcctgaacaaggaggtggtcaggcagctgagcaggcggtacccacagctgcccagggccgtggccaccggcagggtgtacgacatgaacaccggcaccctgaggaactacgaccccaggatcaacctggtgcccgtgaacaggcggctgccccacgccctggtgctgcaccacaacgagcacccacagagcgacttcagctccttcgtgagcaagctgaaaggcaggaccgtgctggtcgtgggcgagaagctgagcgtgcccggcaagatggtggactggctgagcgacaggcccgaggccaccttccgggccaggctggacctcggcatccccggcgacgtgcccaagtacgacatcatcttcgtgaacgtcaggaccccatacaagtaccaccattaccagcagtgcgaggaccacgccatcaagctgagcatgctgaccaagaaggcctgcctgcacctgaaccccggaggcacctgcgtgagcatcggctacggctacgccgacagggccagcgagagcatcattggcgccatcgccaggctgttcaagttcagcagggtgtgcaaacccaagagcagcctggaggaaaccgaggtgctgttcgtgttcatcggctacgaccggaaggccaggacccacaacccctacaagctgagcagcaccctgacaaacatctacaccggcagcaggctgcacgaggccggctgcgcccccagctaccacgtggtcaggggcgatatcgccaccgccaccgagggcgtgatcatcaacgctgccaacagcaagggccagcccggaggcggagtgtgcggcgccctgtacaagaagttccccgagagcttcgacctgcagcccatcgaggtgggcaaggccaggctggtgaagggcgccgctaagcacatcatccacgccgtgggccccaacttcaacaaggtgagcgaggtggaaggcgacaagcagctggccgaagcctacgagagcatcgccaagatcgtgaacgacaataactacaagagcgtggccatcccactgctcagcaccggcatcttcagcggcaacaaggacaggctgacccagagcctgaaccacctgctcaccgccctggacaccaccgatgccgacgtggccatctactgcagggacaagaagtgggagatgaccctgaaggaggccgtggccaggcgggaggccgtggaagagatctgcatcagcgacgactccagcgtgaccgagcccgacgccgagctggtgagggtgcaccccaagagctccctggccggcaggaagggctacagcaccagcgacggcaagaccttcagctacctggagggcaccaagttccaccaggccgctaaggacatcgccgagatcaacgctatgtggcccgtggccaccgaggccaacgagcaggtgtgcatgtacatcctgggcgagagcatgtccagcatcaggagcaagtgccccgtggaggaaagcgaggccagcacaccacccagcaccctgccctgcctgtgcatccacgctatgacacccgagagggtgcagcggctgaaggccagcaggcccgagcagatcaccgtgtgcagctccttcccactgcccaagtacaggatcaccggcgtgcagaagatccagtgcagccagcccatcctgttcagcccaaaggtgcccgcctacatccaccccaggaagtacctggtggagaccccacccgtggacgagacacccgagccaagcgccgagaaccagagcaccgagggcacacccgagcagccacccctgatcaccgaggacgagacaaggacccggaccccagagcccatcattatcgaggaagaggaagaggacagcatcagcctgctgagcgacggccccacccaccaggtgctgcaggtggaggccgacatccacggcccacccagcgtgtccagctccagctggagcatcccacacgccagcgacttcgacgtggacagcctgagcatcctggacaccctggagggcgccagcgtgacctccggcgccaccagcgccgagaccaacagctacttcgccaagagcatggagttcctggccaggcccgtgccagctcccaggaccgtgttcaggaacccaccccacccagctcccaggaccaggaccccaagcctggctcccagcagggcctgcagcaggaccagcctggtgagcaccccacccggcgtgaacagggtgatcaccagggaggaactggaggccctgacacccagcaggacccccagcaggtccgtgagcaggactagtctggtgtccaacccacccggcgtgaacagggtgatcaccagggaggaattcgaggccttcgtggcccagcaacagagacggttcgacgccggcgcctacatcttcagcagcgacaccggccagggacacctgcagcaaaagagcgtgaggcagaccgtgctgagcgaggtggtgctggagaggaccgagctggaaatcagctacgcccccaggctggaccaggagaaggaggaactgctcaggaagaaactgcagctgaaccccaccccagccaacaggagcaggtaccagagcaggaaggtggagaacatgaaggccatcaccgccaggcggatcctgcagggcctgggacactacctgaaggccgagggcaaggtggagtgctacaggaccctgcaccccgtgccactgtacagctccagcgtgaacagggccttctccagccccaaggtggccgtggaggcctgcaacgctatgctgaaggagaacttccccaccgtggccagctactgcatcatccccgagtacgacgcctacctggacatggtggacggcgccagctgctgcctggacaccgccagcttctgccccgccaagctgaggagcttccccaagaaacacagctacctggagcccaccatcaggagcgccgtgcccagcgccatccagaacaccctgcagaacgtgctggccgctgccaccaagaggaactgcaacgtgacccagatgagggagctgcccgtgctggacagcgctgccttcaacgtggagtgcttcaagaaatacgcctgcaacaacgagtactgggagaccttcaaggagaaccccatcaggctgaccgaagagaacgtggtgaactacatcaccaagctgaagggccccaaggccgctgccctgttcgctaagacccacaacctgaacatgctgcaggacatcccaatggacaggttcgtgatggacctgaagagggacgtgaaggtgacacccggcaccaagcacaccgaggagaggcccaaggtgcaggtgatccaggccgctgacccactggccaccgcctacctgtgcggcatccacagggagctggtgaggcggctgaacgccgtgctgctgcccaacatccacaccctgttcgacatgagcgccgaggacttcgacgccatcatcgccgagcacttccagcccggcgactgcgtgctggagaccgacatcgccagcttcgacaagagcgaggatgacgctatggccctgaccgctctgatgatcctggaggacctgggcgtggacgccgagctgctcaccctgatcgaggctgccttcggcgagatcagctccatccacctgcccaccaagaccaagttcaagttcggcgctatgatgaaaagcggaatgttcctgaccctgttcgtgaacaccgtgatcaacattgtgatcgccagcagggtgctgcgggagaggctgaccggcagcccctgcgctgccttcatcggcgacgacaacatcgtgaagggcgtgaaaagcgacaagctgatggccgacaggtgcgccacctggctgaacatggaggtgaagatcatcgacgccgtggtgggcgagaaggccccctacttctgcggcggattcatcctgtgcgacagcgtgaccggcaccgcctgcagggtggccgaccccctgaagaggctgttcaagctgggcaagccactggccgctgacgatgagcacgacgatgacaggcggagggccctgcacgaggaaagcaccaggtggaacagggtgggcatcctgagcgagctgtgcaaggccgtggagagcaggtacgagaccgtgggcaccagcatcatcgtgatggctatgaccacactggccagctccgtcaagagcttctcctacctgaggggggcccctataactctctacggctaacctgaatggactacgacatagtctagtccgccaaggccgccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgacagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagtccctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaactcgagccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgagcaagatctacatcgacgagcggagcaacgccgagatcgtgtgcgaggccatcaagaccatcggcatcgagggcgccaccgccgcccagctgaccaggcagctgaacatggagaagcgggaggtgaacaaggccctgtacgacctgcagaggagcgctatggtgtactccagcgacgacatccctccccggtggttcatgaccaccgaggccgacaagcccgacgccgacgctatggccgacgtgatcatcgacgacgtgagcagggagaagtccatgagggaggaccacaagagcttcgacgacgtgatccccgccaagaagatcatcgactggaagggcgccaaccccgtgaccgtgatcaacgagtactgccagatcaccaggagggactggagcttccggatcgagagcgtgggccccagcaacagccccaccttctacgcctgcgtggacatcgacggcagggtgttcgacaaggccgacggcaagagcaagcgggacgccaagaacaacgccgccaagctggccgtggacaagctgctgggctacgtgatcatccggttctaaacaattggcaagctgcttacatagaactcgcggcgattggcatgccgccttaaaatttttattttattttttcttttcttttccgaatcggattttgtttttaatatttcaaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa